(19) United States
(12) Patent Application Publication
(10) Pub. No.: US 2023/0114151 A1
(43) Pub. Date: Apr. 13, 2023

(54) COMPOSITIONS AND METHODS FOR MODULATING FORKHEAD BOX P3 (FOXP3) GENE EXPRESSION

(71) Applicant: Omega Therapeutics, Inc., Cambridge, MA (US)

(72) Inventors: Timsi Rao, Allston, MA (US); Jodi Kennedy, Dedham, MA (US); Pankaj Mandal, Cambridge, MA (US); Jeremiah D. Farelli, Marblehead, MA (US)

(73) Assignee: Omega Therapeutics, Inc., Cambridge, MA (US)

(21) Appl. No.: 17/929,828

(22) Filed: Sep. 6, 2022

Related U.S. Application Data
(63) Continuation of application No. PCT/US2021/021825, filed on Mar. 11, 2021.
(60) Provisional application No. 62/988,044, filed on Mar. 11, 2020.

(51) Int. Cl. C12N 15/90; C12N 15/11; C12N 9/22; C12N 15/86; C12N 15/62; A61P 37/06; A61K 45/06

(52) U.S. Cl. CPC C12N 15/907; C12N 15/11; C12N 9/22; C12N 15/86; C12N 15/62; A61P 37/06; A61K 45/06; C12N 2310/20; C12N 2800/80; C12N 2750/14143; C07K 2319/81

(57) ABSTRACT

The present invention provides agents and compositions for modulating expression (e.g., enhanced or reduced expression) of a forkhead box P3 (FOXP3) gene by targeting a FOXP3 expression control region and methods of use thereof for treating a FOXP3 associated disorder, such as an autoimmune disease, e.g., IPEX syndrome.

### RELATED APPLICATION

This application is a 35 § U.S.C. 111(a) continuation application of Internatonal Application No. PCT/US2021/021825, filed on Mar. 11, 2021, which claims the benefit of priority to U.S. Provisional Application No. 62/988,044, filed on Mar. 11, 2020. The entire contents of each of the foregoing applications are incorporated herein by reference.

### SEQUENCE LISTING

This instant application contains a Sequence Listing which has been submitted electronically in XML file format and is hereby incorporated by reference in its entirety. Said XML copy, created on Sep. 6, 2022, is named 131717-00402_SL.xml and is 1,358,498 bytes in size.

### BACKGROUND OF THE INVENTION

A healthy immune system defends the body against disease and infection. But if the immune system malfunctions, it mistakenly attacks healthy cells, tissues, and organs. These attacks, characterizing autoimmune diseases or disorders, can affect any part of the body, weakening bodily function and even turning life-threatening. Some of the more common autoimmune diseases include IPEX syndrome (IPEX), type 1 diabetes, multiple sclerosis, systemic lupus erythematosus (SLE), and rheumatoid arthritis (RA).

Collectively, these diseases affect more than 24 million people in the United States (see Progress in Autoimmune Diseases Research, https://www.niaid.nih.gov/sites/default/files/adccfinal.pdf). An additional eight million people have auto-antibodies, blood molecules that indicate a person's chance of developing an autoimmune disease. Autoimmune diseases are becoming more prevalent.

Treatment depends on the disease, but in most cases one important goal is to reduce inflammation. Corticosteroids or other drugs that reduce immune response are usually prescribed.

Regulatory T cells (Tregs) are a specialized subpopulation of T cells that act to suppress immune response, thereby maintaining homeostasis and self-tolerance. It has been shown that Tregs are able to inhibit T cell proliferation and cytokine production and play an important role in preventing or treating autoimmune disease. Forkhead box P3 (FOXP3) is a master transcription factor that controls the differentiation of naïve T-cells into regulatory T-cells (Tregs) and forced overexpression of FOXP3 has been shown to confer the Treg phenotype to T-cells.

In vitro generation of Tregs has been an important effort in the field of ex vivo therapy targeting autoimmune disorders. However, many of the strategies to produce Tregs either do not lead to sustained expression of genes that lead to Tregs or give rise to Tregs that do not have suppression phenotype.

Accordingly, there is a need in the art for compositions and methods that treat an autoimmune disease, such as IPEX syndrome.

### SUMMARY OF THE INVENTION

The present invention provides agents and compositions for modulating the expression (e.g., enhancing or reducing the expression) of the Forkhead box P3 (FOXP3) gene by targeting a FOXP3 expression control region. The FOXP3 gene may be in a cell, e.g., a mammalian cell, such as a mammalian somatic cell, e.g., a human or mouse somatic cell, e.g., a naïve T-cell. The present invention also provides methods of using the agents and compositions of the invention for modulating the expression of a FOXP3 gene in, or for treating, a subject who would benefit from modulating the expression of a FOXP3 gene, e.g., a subject suffering or prone to suffering from a FOXP3-associated disease.

Accordingly, in one aspect, the present invention provides a site-specific forkhead box P3(FOXP3) disrupting agent, comprising a site-specific FOXP3 targeting moiety which targets a FOXP3 expression control region.

In one embodiment, the site-specific FOXP3 targeting moiety comprises a polymeric molecule. The polymeric molecule may comprise a polyamide, a polynucleotide, a polynucleotide encoding a DNA-binding domain, or fragment thereof, that specifically binds to the FOXP3 expression control region, or a peptide nucleic acid (PNA).

In yet another embodiment, the expression control region comprises a region upstream of a FOXP3 transcription start site (TSS).

In one embodiment, the expression control region comprises one or more FOXP3 associated anchor sequences within an anchor sequence-mediated conjunction comprising a first and a second FOXP3-associated anchor sequence.

In another embodiment, the FOXP3-associated anchor sequence comprises a CCCTC-binding factor (CTCF) binding motif.

In yet another embodiment, the FOXP3-associated anchor sequence-mediated conjunction comprises one or more transcriptional control elements internal to the conjunction. In one embodiment, the FOXP3-associated anchor sequence-mediated conjunction comprises one or more transcriptional control elements external to the conjunction.

In one embodiment, the FOXP3-associated anchor sequence is located within about 500 kb of the transcriptional control element. In another embodiment, the FOXP3-associated anchor sequence is located within about 300 kb of the transcriptional control element. In still another embodiment, the anchor sequence is located within 10 kb of the transcriptional control element.

In another embodiment, the expression control region comprises a FOXP3-specific transcriptional control element. In still another embodiment, the transcriptional control element comprises a FOXP3 promoter. In yet another embodiment, the transcriptional control element comprises a transcriptional enhancer. In still another embodiment, the transcriptional control element comprises a transcriptional repressor.

In one embodiment, the site-specific FOXP3 disrupting agent includes a nucleotide sequence having at least 85%, 86%, 87%, 88%, 89%, 90%, 91%, 92%, 93%, 94%, 95%, 96%, 97%, 98%, 99%, or 100% nucleotide identity to the entire nucleotide sequence of any one of the nucleotide sequences in Table 2.

In another embodiment, the site-specific FOXP3 disrupting agent includes a polynucleotide encoding a DNA-binding domain, or fragment thereof, of a zinc finger polypeptide (ZNF) or a transcription activator-like effector (TALE) polypeptide that specifically binds to the FOXP3 expression control region.

In one embodiment, the DNA-binding domain of the TALE or ZNF polypeptide comprises an amino acid sequence having at least about 85%, 86%, 87%, 88%, 89%, 90%, 91%, 92%, 93%, 94%, 95%, 96%, 97%, 98%, 99%, or 100% amino acid identity to the entire amino acid sequence of any one of the amino acid sequences listed in Table 1B.

In still another embodiment, the site-specific FOXP3 disrupting agent includes a nucleotide modification, e.g., a deoxy-nucleotide, a 3′-terminal deoxy-thymine (dT) nucleotide, a 2′-O-methyl modified nucleotide, a 2′-fluoro modified nucleotide, a 2′-deoxy-modified nucleotide, an abasic nucleotide, a nucleotide comprising a 5′-phosphorothioate group, a nucleotide comprising a 5′-methylphosphonate group, a nucleotide comprising a 3′-phosphorothioate group, or a nucleotide comprising a 3′-methylphosphonate group.

In yet another embodiment, the polymeric molecule comprises a peptide nucleic acid (PNA).

In one aspect, the present invention provides a vector. The vector includes the site-specific FOXP3 disrupting agent of various embodiments of the above aspects or any other aspect of the invention delineated herein. In one embodiment, the vector is a viral expression vector.

In another aspect, the present invention provides a cell. The cell provides the site-specific FOXP3 disrupting agent or the vector of various embodiments of the above aspects or any other aspect of the invention delineated therein.

In one embodiment, the site-specific FOXP3 disrupting agent is present in a composition. In another embodiment, the composition comprises a pharmaceutical composition. In still another embodiment, the pharmaceutical composition comprises a lipid formulation. In yet another embodiment, the lipid formulation comprises one or more cationic lipids, one or more non-cationic lipids, one or more cholesterol-based lipids, or one or more PEG-modified lipids, or combinations of any of the foregoing. In one embodiment, the pharmaceutical composition comprises a lipid nanoparticle.

In another aspect, the present invention provides a site-specific FOXP3 disrupting agent. The site-specific FOXP3 disrupting agent includes a nucleic acid molecule encoding a fusion protein, the fusion protein comprising a site-specific FOXP3 targeting moiety which targets a FOXP3 expression control region and an effector molecule.

In one embodiment, the site-specific FOXP3 targeting moiety comprises a polynucleotide encoding a DNA-binding domain, or fragment thereof, of a zinc finger polypeptide (ZNF) or a transcription activator-like effector (TALE) polypeptide that specifically binds to the FOXP3 expression control region.

In another embodiment, the effector molecule comprises a polypeptide or a nucleic acid molecule encoding a polypeptide. In still another embodiment, the fusion protein comprises a peptide-nucleic acid fusion.

In yet another embodiment, the effector is selected from the group consisting of a nuclease, a physical blocker, an epigenetic recruiter, and an epigenetic CpG modifier, and combinations of any of the foregoing.

In one embodiment, the effector comprises a CRISPR associated protein (Cas) polypeptide or nucleic acid molecule encoding the Cas polypeptide. In another embodiment, Cas polypeptide is an enzymatically inactive Cas polypeptide. In still another embodiment, the site-specific FOXP3 disrupting agent further includes a catalytically active domain of human exonuclease 1 (hEXO1).

In another embodiment, epigenetic recruiter comprises a transcriptional enhancer or a transcriptional repressor.

In one embodiment, the transcriptional enhancer is a VPR (VP64-p65-Rta).

In one embodiment, the VPR comprises an amino acid sequence having at least about 85%, 86%, 87%, 88%, 89%, 90%, 91%, 92%, 93%, 94%, 95%, 96%, 97%, 98%, 99%, or 100% amino acid identity to the entire amino acid sequence of

(SEQ ID NO: 64)
DALDDFDLDMLGSDALDDFDLDMLGSDALDDFDLDMLGSDALDDFDLDML
SGGPKKKRKVGSQYLPDTDDRHRIEEKRKRTYETFKSIMKKSPFSGPTDP
RPPPRRIAVPSRSSASVPKPAPQPYPFTSSLSTINYDEFPTMVFPSGQIS
QASALAPAPPQVLPQAPAPAPAPAMVSALAQAPAPVPVLAPGPPQAVAPP
APKPTQAGEGTLSEALLQLQFDDEDLGALLGNSTDPAVFTDLASVDNSEF
QQLLNQGIPVAPHTTEPMLMEYPEAITRLVTGAQRPPDPAPAPLGAPGLP
NGLLSGDEDFSSIADMDFSALLGSGSGSRDSREGMFLPKPEAGSAISDVF
EGREVCQPKRLRPFHPPGSPWANRPLPASLAPTPTGPVHEPVGSLTPAPV
PQPLDPAPAVTPEASHLLEDPDEETSQAVKALREMADTVIPQKEEAAICG
QMDLSHPPPRGHLDELTTTLESMTEDLNLDSPLTPELNEILDTFLNDECL
LHAMHISTGLSIFDTSLF.

In one embodiment, the transcriptional enhancer comprises two, three, four, or five VPRs.

In one embodiment, the transcriptional enhancer is a p300.

In one embodiment, the p300 comprises an amino acid sequence having at least about 85% 85%, 86%, 87%, 88%, 89%, 90%, 91%, 92%, 93%, 94%, 95%, 96%, 97%, 98%, 99%, or 100% identity to the entire amino acid sequence of

(SEQ ID NO: 65)
IFKPEELRQALMPTLEALYRQDPESLPFRQPVDPQLLGIPDYFDIVKSPM
DLSTIKRKLDTGQYQEPWQYVDDIWLMFNNAWLYNRKTSRVYKYCSKLSE
VFEQEIDPVMQSLGYCCGRKLEFSPQTLCCYGKQLCTIPRDATYYSYQNR
YHFCEKCFNEIQGESVSLGDDPSQPQTTINKEQFSKRKNDTLDPELFVEC
TECGRKMHQICVLHHEIIWPAGFVCDGCLKKSARTRKENKFSAKRLPSTR
LGTFLENRVNDFLRRQNHPESGEVTVRVVHASDKTVEVKPGMKARFVDSG
EMAESFPYRTKALFAFEEIDGVDLCFFGMHVQEYGSDCPPPNQRRVYISY
LDSVHFFRPKCLRTAVYHEILIGYLEYVKKLGYTTGHIWACPPSEGDDYI
FHCHPPDQKIPKPKRLQEWYKKMLDKAVSERIVHDYKDIFKQATEDRLTS
AKELPYFEGDFWPNVLEESIKELEQEEEERKREENTSNESTDVTKGDSKN
AKKKNNKKTSKNKSSLSRGNKKKPGMPNVSNDLSQKLYATMEKHKEVFFV
IRLIAGPAANSLPPIVDPDPLIPCDLMDGRDAFLTLARDKHLEFSSLRRA
QWSTMCMLVELHTQSQD.

In still another embodiment, the epigenetic CpG modifier comprises a DNA methylase, a DNA demethylase, a histone modifying agent, a histone transacetylase, or a histone deacetylase.

In one embodiment, the effector molecule comprises a zinc finger polypeptide. In another embodiment, the effector molecule comprises a Transcription activator-like effector nuclease (TALEN) polypeptide.

In some embodiments, the site-specific FOXP3 disrupting agent further comprises a second nucleic acid molecule encoding a second fusion protein, wherein the second fusion comprises a second site-specific FOXP3 targeting moiety which targets a second FOXP3 expression control region and a second effector molecule, wherein the second FOXP3 expression control region is different than the FOXP3 expression control region.

In one embodiment, the second effector is different than the first effector.

In one embodiment, the second effector is the same as the first effector.

In one embodiment, the fusion protein and the second fusion protein are operably linked.

In one embodiment, the fusion protein and the second fusion protein comprise an amino acid sequence that has at least about 85%, 86%, 87%, 88%, 89%, 90%, 91%, 92%, 93%, 94%, 95%, 96%, 97%, 98%, 99%, or 100% amino acid sequence identity to the entire amino acid sequence of a polypeptide selected from the group consisting of dCas9-P300, and dCas9-VPR.

In one embodiment, the fusion protein is encoded by a polynucleotide comprising a nucleotide sequence having at least about 85%, 86%, 87%, 88%, 89%, 90%, 91%, 92%, 93%, 94%, 95%, 96%, 97%, 98%, 99%, or 100% nucleotide sequence identity to the entire nucleotide sequence of a polynucleotide selected from the group consisting of dCas9-P300 mRNA, and dCas9-VPR mRNA.

In one aspect, the present invention provides a site-specific FOXP3 disrupting agent. The disrupting agent includes a nucleic acid molecule encoding a fusion protein, wherein the fusion protein comprises an amino acid sequence having at least about 85%, 86%, 87%, 88%, 89%, 90%, 91%, 92%, 93%, 94%, 95%, 96%, 97%, 98%, 99%, or 100% amino acid identity to the entire amino acid sequence of a polypeptide selected from the group consisting of dCas9-P300, and dCas9-VPR.

In one aspect, the present invention provides a site-specific FOXP3 disrupting agent. The site-specific FOXP3 disrupting agent comprises a polynucleotide encoding the amino acid sequence of dCas-P300 comprising the amino acid sequence of

(SEQ ID NO: 10)
MAPKKKRKVGIHGVPAADKKYSIGLAIGTNSVGWAVITDEYKVPSKKFKVLGNTDRHSIKK
NLIGALLFDSGETAEATRLKRTARRRYTRRKNRICYLQEIFSNEMAKVDDSFFHRLEESFLVE
EDKKHERHPIFGNIVDEVAYHEKYPTIYHLRKKLVDSTDKADLRLIYLALAHMIKFRGHFLIE
GDLNPDNSDVDKLFIQLVQTYNQLFEENPINASGVDAKAILSARLSKSRRLENLIAQLPGEKK
NGLFGNLIALSLGLTPNFKSNFDLAEDAKLQLSKDTYDDDLDNLLAQIGDQYADLFLAAKNL
SDAILLSDILRVNTEITKAPLSASMIKRYDEHHQDLTLLKALVRQQLPEKYKEIFFDQSKNGYA
GYIDGGASQEEFYKFIKPILEKMDGTEELLVKLNREDLLRKQRTFDNGSIPHQIHLGELHAILR
RQEDFYPFLKDNREKIEKILTFRIPYYVGPLARGNSRFAWMTRKSEETITPWNFEEVVDKGAS
AQSFIERMTNFDKNLPNEKVLPKHSLLYEYFTVYNELTKVKYVTEGMRKPAFLSGEQKKAIV
DLLFKTNRKVTVKQLKEDYFKKIECFDSVEISGVEDRFNASLGTYHDLLKIIKDKDFLDNEEN
EDILEDIVLTLTLFEDREMIEERLKTYAHLFDDKVMKQLKRRRYTGWGRLSRKLINGIRDKQS
GKTILDFLKSDGFANRNFMQLIHDDSLTFKEDIQKAQVSGQGDSLHEHIANLAGSPAIKKGIL
QTVKVVDELVKVMGRHKPENIVIEMARENQTTQKGQKNSRERMKRIEEGIKELGSQILKEHP
VENTQLQNEKLYLYYLQNGRDMYVDQELDINRLSDYDVAAIVPQSFLKDDSIDNKVLTRSD
KARGKSDNVPSEEVVKKMKNYWRQLLNAKLITQRKFDNLTKAERGGLSELDKAGFIKRQLV
ETRQITKHVAQILDSRMNTKYDENDKLIREVKVITLKSKLVSDFRKDFQFYKVREINNYHHA
HDAYLNAVVGTALIKKYPKLESEFVYGDYKVYDVRKMIAKSEQEIGKATAKYFFYSNIMNFF
KTEITLANGEIRKRPLIETNGETGEIVWDKGRDFATVRKVLSMPQVNIVKKTEVQTGGFSKESI
LPKRNSDKLIARKKDWDPKKYGGFDSPTVAYSVLVVAKVEKGKSKKLKSVKELLGITIMERS
SFEKNPIDFLEAKGYKEVKKDLIIKLPKYSLFELENGRKRMLASAGELQKGNELALPSKYVNF
LYLASHYEKLKGSPEDNEQKQLFVEQHKHYLDEIIEQISEFSKRVILADANLDKVLSAYNKHR
DKPIREQAENIIHLFTLTNLGAPAAFKYFDTTIDRKRYTSTKEVLDATLIHQSITGLYETRIDLS
QLGGDKRPAATKKAGQAKKKKGRAIFKPEELRQALMPTLEALYRQDPESLPFRQPVDPQLL
GIPDYFDIVKSPMDLSTIKRKLDTGQYQEPWQYVDDIWLMFNNAWLYNRKTSRVYKYCSKL
SEVFEQEIDPVMQSLGYCCGRKLEFSPQTLCCYGKQLCTIPRDATYYSYQNRYHFCEKCFNEI
QGESVSLGDDPSQPQTTINKEQFSKRKNDTLDPELFVECTECGRKMHQICVLHHEIIWPAGFV
CDGCLKKSARTRKENKFSAKRLPSTRLGTFLENRVNDFLRRQNHPESGEVTVRVVHASDKTV
EVKPGMKARFVDSGEMAESFPYRTKALFAFEEIDGVDLCFFGMHVQEYGSDCPPPNQRRVYI
SYLDSVHFFRPKCLRTAVYHEILIGYLEYVKKLGYTTGHIWACPPSEGDDYIFHCHPPDQKIPK
PKRLQEWYKKMLDKAVSERIVHDYKDIFKQATEDRLTSAKELPYFEGDFWPNVLEESIKELE
QEEEERKREENTSNESTDVTKGDSKNAKKKNNKKTSKNKSSLSRGNKKKPGMPNVSNDLSQ
KLYATMEKHKEVFFVIRLIAGPAANSLPPIVDPDPLIPCDLMDGRDAFLTLARDKHLEFSSLRR
AQWSTMCMLVELHTQSQDSGGKRPAATKKAGQAKKKKGSYPYDVPDYA.

In another aspect, the present invention provides a site-specific FOXP3 disrupting agent. The site-specific FOXP3 disrupting agent comprises a polynucleotide encoding the amino acid sequence of dCas-VPR comprising the amino acid sequence of

(SEQ ID NO: 11)
MAPKKKRKVGIHGVPAADKKYSIGLAIGTNSVGWAVITDEYKVPSKKFKVLGNTDRHSIKK
NLIGALLFDSGETAEATRLKRTARRRYTRRKNRICYLQEIFSNEMAKVDDSFFHRLEESFLVE
EDKKHERHPIFGNIVDEVAYHEKYPTIYHLRKKLVDSTDKADLRLIYLALAHMIKFRGHFLIE
GDLNPDNSDVDKLFIQLVQTYNQLFEENPINASGVDAKAILSARLSKSRRLENLIAQLPGEKK
NGLFGNLIALSLGLTPNFKSNFDLAEDAKLQLSKDTYDDDLDNLLAQIGDQYADLFLAAKNL
SDAILLSDILRVNTEITKAPLSASMIKRYDEHHQDLTLLKALVRQQLPEKYKEIFFDQSKNGYA
GYIDGGASQEEFYKFIKPILEKMDGTEELLVKLNREDLLRKQRTFDNGSIPHQIHLGELHAILR
RQEDFYPFLKDNREKIEKILTFRIPYYVGPLARGNSRFAWMTRKSEETITPWNFEEVVDKGAS
AQSFIERMTNFDKNLPNEKVLPKHSLLYEYFTVYNELTKVKYVTEGMRKPAFLSGEQKKAIV
DLLFKTNRKVTVKQLKEDYFKKIECFDSVEISGVEDRFNASLGTYHDLLKIIKDKDFLDNEEN
EDILEDIVLTLTLFEDREMIEERLKTYAHLFDDKVMKQLKRRRYTGWGRLSRKLINGIRDKQS
GKTILDFLKSDGFANRNFMQLIHDDSLTFKEDIQKAQVSGQGDSLHEHIANLAGSPAIKKGIL
QTVKVVDELVKVMGRHKPENIVIEMARENQTTQKGQKNSRERMKRIEEGIKELGSQILKEHP
VENTQLQNEKLYLYYLQNGRDMYVDQELDINRLSDYDVAAIVPQSFLKDDSIDNKVLTRSD
KARGKSDNVPSEEVVKKMKNYWRQLLNAKLITQRKFDNLTKAERGGLSELDKAGFIKRQLV
ETRQITKHVAQILDSRMNTKYDENDKLIREVKVITLKSKLVSDFRKDFQFYKVREINNYHHA
HDAYLNAVVGTALIKKYPKLESEFVYGDYKVYDVRKMIAKSEQEIGKATAKYFFYSNIMNFF
KTEITLANGEIRKRPLIETNGETGEIVWDKGRDFATVRKVLSMPQVNIVKKTEVQTGGFSKESI
LPKRNSDKLIARKKDWDPKKYGGFDSPTVAYSVLVVAKVEKGKSKKLKSVKELLGITIMERS
SFEKNPIDFLEAKGYKEVKKDLIIKLPKYSLFELENGRKRMLASAGELQKGNELALPSKYVNF
LYLASHYEKLKGSPEDNEQKQLFVEQHKHYLDEIIEQISEFSKRVILADANLDKVLSAYNKHR
DKPIREQAENIIHLFTLTNLGAPAAFKYFDTTIDRKRYTSTKEVLDATLIHQSITGLYETRIDLS
QLGGDKRPAATKKAGQAKKKKGRADALDDFDLDMLGSDALDDFDLDMLGSDALDDFDLD
MLGSDALDDFDLDMLSGGPKKKRKVGSQYLPDTDDRHRIEEKRKRTYETFKSIMKKSPFSGP
TDPRPPPRRIAVPSRSSASVPKPAPQPYPFTSSLSTINYDEFPTMVFPSGQISQASALAPAPPQVL
PQAPAPAPAPAMVSALAQAPAPVPVLAPGPPQAVAPPAPKPTQAGEGTLSEALLQLQFDDED
LGALLGNSTDPAVFTDLASVDNSEFQQLLNQGIPVAPHTTEPMLMEYPEAITRLVTGAQRPPD
PAPAPLGAPGLPNGLLSGDEDFSSIADMDFSALLGSGSGSRDSREGMFLPKPEAGSAISDVFEG

REVCQPKRIRPFHPPGSPWANRPLPASLAPTPTGPVHEPVGSLTPAPVPQPLDPAPAVTPEASH
LLEDPDEETSQAVKALREMADTVIPQKEEAAICGQMDLSHPPPRGHLDELTTTLESMTEDLN
LDSPLTPELNEILDTFLNDECLLHAMHISTGLSIFDTSLFSGGKRPAATKKAGQAKKKKGSYP
YDVPDYA.

In one aspect, the present invention provides a vector. The vector includes a nucleic acid molecule encoding the site-specific FOXP3 disrupting agent of various embodiments of the above aspects or any other aspect of the invention delineated herein. In one embodiment, the vector is a viral expression vector.

In another aspect, the present invention provides a cell. The cell includes the site-specific FOXP3 disrupting agent or the vector of various embodiments of the above aspects or any other aspects of the invention delineated herein.

In one embodiment, the site-specific FOXP3 disrupting agent is present in a composition. In another embodiment, the composition comprises a pharmaceutical composition. In still another embodiment, the pharmaceutical composition comprises a lipid formulation. In yet another embodiment, the lipid formulation comprises one or more cationic lipids, one or more non-cationic lipids, one or more cholesterol-based lipids, or one or more PEG-modified lipids, or combinations of any of the foregoing. In one embodiment, the pharmaceutical composition comprises a lipid nanoparticle.

In one aspect, the present invention provides a method of modulating expression of forkhead box P3 (FOXP3) in a cell. The method includes contacting the cell with a site-specific FOXP3 disrupting agent, the disrupting agent comprising a site-specific FOXP3 targeting moiety which targets a FOXP3 expression control region, and an effector molecule, thereby modulating expression of FOXP3 in the cell.

In one embodiment, the modulation of expression is enhanced expression of FOXP3 in the cell. In another embodiment, the modulation of expression is reduced expression of FOXP3 in the cell. In still another embodiment, the site-specific FOXP3 targeting moiety comprises a polymeric molecule. In yet another embodiment, the polymeric molecule comprises a polyamide. In one embodiment, the polymeric molecule comprises a polynucleotide.

In another embodiment, the expression control region comprises a region upstream of FOXP3 transcription start site (TSS).

In still another embodiment, the expression control region comprises one or more FOXP3-associated anchor sequences within an anchor sequence-mediated conjunction comprising a first and a second FOXP3-associated anchor sequence. In yet another embodiment, the FOXP3-associated anchor sequence comprises a CCCTC-binding factor (CTCF) binding motif.

In one embodiment, the FOXP3-associated anchor sequence-mediated conjunction comprises one or more transcriptional control elements internal to the conjunction. In another embodiment, the FOXP3-associated anchor sequence-mediated conjunction comprises one or more transcriptional control elements external to the conjunction.

In another embodiment, the anchor sequence is located within about 500 kb of the transcriptional control element. In another embodiment, the anchor sequence is located within about 300 kb of the transcriptional control element. In still another embodiment, the anchor sequence is located within 10 kb of the transcriptional control element.

In one embodiment, the expression control region comprises a FOXP3-specific transcriptional element. In another embodiment, the transcriptional element comprises a FOXP3 promoter. In still another embodiment, the transcriptional control element comprises transcriptional enhancer. In yet another embodiment, the transcriptional control element comprises a transcriptional repressor.

In another embodiment, the site-specific FOXP3 disrupting agent comprises a nucleotide sequence having at least 85%, 86%, 87%, 88%, 89%, 90%, 91%, 92%, 93%, 94%, 95%, 96%, 97%, 98%, 99%, or 100% nucleotide identity to the entire nucleotide sequence of any of the nucleotide sequences in Table 2.

In one embodiment, the site-specific FOXP3 disrupting agent comprises a polynucleotide encoding a DNA-binding domain, or fragment thereof, of a zinc finger polypeptide (ZNF) or a transcription activator-like effector (TALE) polypeptide that specifically binds to the FOXP3 expression control region.

In some embodiments, the DNA-binding domain of the TALE or ZNF comprises an amino acid sequence having at least 85%, 86%, 87%, 88%, 89%, 90%, 91%, 92%, 93%, 94%, 95%, 96%, 97%, 98%, 99%, or 100% amino acid identity to the entire amino acid sequence of an amino acid sequence selected from the amino acid sequences listed in Table 1B.

In another embodiment, the site-specific FOXP3 disrupting agent comprises a nucleotide modification.

In still another embodiment, the polymeric molecule comprises a peptide nucleic acid (PNA).

In one embodiment, the effector molecule comprises a polypeptide. In another embodiment, the polypeptide comprises a fusion protein comprising the site-specific FOXP3 targeting moiety which targets a FOXP3 expression regulatory region, and the effector molecule. In still another embodiment, the fusion protein comprises a peptide-nucleic acid fusion molecule.

In another embodiment, the effector is selected from the group consisting of a nuclease, a physical blocker, an epigenetic recruiter, and an epigenetic CpG modifier, and combinations of any of the foregoing. In still another embodiment, the effector comprises a CRISPR associated protein (Cas) polypeptide or nucleic acid molecule encoding the Cas polypeptide. In yet another embodiment, the Cas polypeptide is an enzymatically inactive Cas polypeptide. In one embodiment, the effector further comprises a catalytically active domain of human exonuclease 1 (hEXO1).

In one embodiment, the epigenetic recruiter comprises a transcriptional enhancer or a transcriptional repressor.

In some embodiments, the transcriptional enhancer is a VPR.

In some embodiments, the VPR comprises an amino acid sequence having at least about 85%, 86%, 87%, 88%, 89%, 90%, 91%, 92%, 93%, 94%, 95%, 96%, 97%, 98%, 99%, or 100% amino acid identity to the entire amino acid sequence of

(SEQ ID NO: 64)
DALDDFDLDMLGSDALDDFDLDMLGSDALDDFDLDMLGSDALDDFDLDM
LSGGPKKKRKVGSQYLPDTDDRHRIEEKRKRTYETFKSIMKKSPFSGPT
DPRPPPRRIAVPSRSSASVPKPAPQPYPFTSSLSTINYDEFPTMVFPSG
QISQASALAPAPPQVLPQAPAPAPAPAMVSALAQAPAPVPVLAPGPPQA
VAPPAPKPTQAGEGTLSEALLQLQFDDEDLGALLGNSTDPAVFTDLASV
DNSEFQQLLNQGIPVAPHTTEPMLMEYPEAITRLVTGAQRPPDPAPAPL
GAPGLPNGLLSGDEDFSSIADMDFSALLGSGSGSRDSREGMFLPKPEAG
SAISDVFEGREVCQPKRLRPFHPPGSPWANRPLPASLAPTPTGPVHEPV
GSLTPAPVPQPLDPAPAVTPEASHLLEDPDEETSQAVKALREMADTVIP
QKEEAAICGQMDLSHPPPRGHLDELTTTLESMTEDLNLDSPLTPELNEI
LDTFLNDECLLHAMHISTGLSIFDTSLF.

In some embodiments, the transcriptional enhancer comprises two, three, four, or five VPRs.

In some embodiments, the transcriptional enhancer is a p300.

In some embodiments, the p300 has an amino acid sequence having at least about 85%, 86%, 87%, 88%, 89%, 90%, 91%, 92%, 93%, 94%, 95%, 96%, 97%, 98%, 99%, or 100% amino acid identity to the entire amino acid sequence of

(SEQ ID NO: 65)
IFKPEELRQALMPTLEALYRQDPESLPFRQPVDPQLLGIPDYFDIVKSP
MDLSTIKRKLDTGQYQEPWQYVDDIWLMFNNAWLYNRKTSRVYKYCSKL
SEVFEQEIDPVMQSLGYCCGRKLEFSPQTLCCYGKQLCTIPRDATYYSY
QNRYHFCEKCFNEIQGESVSLGDDPSQPQTTINKEQFSKRKNDTLDPEL
FVECTECGRKMHQICVLHHEIIWPAGFVCDGCLKKSARTRKENKFSAKR
LPSTRLGTFLENRVNDFLRRQNHPESGEVTVRVVHASDKTVEVKPGMKA
RFVDSGEMAESFPYRTKALFAFEEIDGVDLCFFGMHVQEYGSDCPPPNQ
RRVYISYLDSVHFFRPKCLRTAVYHEILIGYLEYVKKLGYTTGHIWACP
PSEGDDYIFHCHPPDQKIPKPKRLQEWYKKMLDKAVSERIVHDYKDIFK
QATEDRLTSAKELPYFEGDFWPNVLEESIKELEQEEEERKREENTSNES
TDVTKGDSKNAKKKNNKKTSKNKSSLSRGNKKKPGMPNVSNDLSQKLYA
TMEKHKEVFFVIRLIAGPAANSLPPIVDPDPLIPCDLMDGRDAFLTLAR
DKHLEFSSLRRAQWSTMCMLVELHTQSQD.

In another embodiment, the epigenetic CpG modifier comprises a DNA methylase, a DNA demethylase, a histone modifying agent, a histone transacetylase, or a histone deacetylase.

In still another embodiment, the effector molecule comprises a zinc finger polypeptide.

In yet another embodiment, the effector molecule comprises a Transcription activator-like effector nuclease (TALEN) polypeptide.

In one embodiment, the fusion protein comprises an enzymatically inactive Cas polypeptide and an epigenetic recruiter polypeptide.

In another embodiment, the fusion protein comprises an enzymatically Cas polypeptide and an epigenetic CpG modifier polypeptide.

In some embodiments, the site-specific FOXP3 disrupting agent further comprises a second nucleic acid molecule encoding a second fusion protein, wherein the second fusion comprises a second site-specific FOXP3 targeting moiety which targets a second FOXP3 expression control region and a second effector molecule, wherein the second FOXP3 expression control region is different than the FOXP3 expression control region.

In one embodiment, the second effector is different than the first effector.

In one embodiment, the second effector is the same as the first effector.

In one embodiment, the fusion protein and the second fusion protein are operably linked.

In one embodiment, the fusion protein and the second fusion protein comprise an amino acid sequence that has at least about 85%, 86%, 87%, 88%, 89%, 90%, 91%, 92%, 93%, 94%, 95%, 96%, 97%, 98%, 99%, or 100% amino acid sequence identity to the entire amino acid sequence of a polypeptide selected from the group consisting of dCas9-P300, and dCas9-VPR.

In one embodiment, the fusion protein is encoded by a polynucleotide comprising a nucleotide sequence having at least about 85%, 86%, 87%, 88%, 89%, 90%, 91%, 92%, 93%, 94%, 95%, 96%, 97%, 98%, 99%, or 100% nucleotide sequence identity to the entire nucleotide sequence of a polynucleotide selected from the group consisting of dCas9-P300 mRNA, and dCas9-VPR mRNA.

In one aspect, the present invention provides a site-specific FOXP3 disrupting agent. The disrupting agent includes a nucleic acid molecule encoding a fusion protein, wherein the fusion protein comprises an amino acid sequence having at least about 85%, 86%, 87%, 88%, 89%, 90%, 91%, 92%, 93%, 94%, 95%, 96%, 97%, 98%, 99%, or 100% amino acid identity to the entire amino acid sequence of a polypeptide selected from the group consisting of dCas9-P300, and dCas9-VPR.

In one embodiment, the site-specific disrupting agent, the effector, or both the site-specific disrupting agent and the effector are present in a vector. In another embodiment, the site-specific disrupting agent and the effector are present in the same vector. In still another embodiment, the site-specific disrupting agent and the effector are present in different vectors. In yet another embodiment, the vector is a viral expression vector.

In one embodiment, the site-specific disrupting agent, the effector, or both the site-specific disrupting agent and the effector are present in a composition. In another embodiment, the site-specific disrupting agent and the effector are present in the same composition. In still another embodiment, the site-specific disrupting agent and the effector are present in different compositions.

In yet another embodiment, the composition comprises a pharmaceutical composition. In one embodiment, the pharmaceutical composition comprises a lipid formulation. In another embodiment, the lipid formulation comprises one or more cationic lipids, one or more non-cationic lipids, one or more cholesterol-based lipids, or one or more PEG-modified lipids, or combinations of any of the foregoing. In still another embodiment, the pharmaceutical composition comprises a lipid nanoparticle.

In another embodiment, the cell is a mammalian cell. In still another embodiment, the mammalian cell is a somatic cell. In yet another embodiment, the mammalian cell is a primary cell.

In one embodiment, the contacting is performed in vitro. In another embodiment, the contacting is performed in vivo. In still another embodiment, the contacting is performed ex vivo.

In one embodiment, the method further includes administering the cell to a subject.

In another embodiment, the cell is within a subject.

In still another embodiment, the subject has a FOXP3-associated disease. In yet another rembodiment, the FOXP3-associated disease is selected from the group consisting of IPEX syndrome (IPEX), type 1 diabetes, multiple sclerosis, systemic lupus erythematosus (SLE), and rheumatoid arthritis (RA).

In one aspect, the present invention provides a method for treating a subject having a FOXP3-associated disease. The method includes administering to the subject a therapeutically effective amount of a site-specific FOXP3 disrupting agent, the disrupting agent comprising a site-specific FOXP3 targeting moiety which targets a FOXP3 expression control region, and an effector molecule, thereby treating the subject. In one embodiment, the FOXP3-associated disease is IPEX syndrome and the site-specific FOXP3 disrupting agent increases expression of FOXP3 in the subject. In another embodiment, the site-specific FOXP3 disrupting agent and the effector molecule are administered to the subject concurrently. In still another embodiment, the site-specific FOXP3 disrupting agent and the effector molecule are administered to the subject sequentially. In one embodiment, the effector molecule is administered to the subject prior to administration of the site-specific FOXP3 disrupting agent. In another embodiment, the site-specific FOXP3 disrupting agent is administered to the subject prior to administration of the effector molecule.

In various embodiments of the above aspects or any other aspects of the invention delineated herein, the cell is an immune cell. In one embodiment, the immune cell is a naïve T cell or a regulatory T cell (Treg). In another embodiment,

In one embodiment, the site-specific FOXP3 disrupting agent of the invention includes a first nucleotide sequence having at least 85%, 90%, 91%, 92%, 93%, 94%, 95%, 96%, 97%, 98%, 99%, or 100% nucleotide identity to the entire nucleotide sequence of GD-28448, a second nucleotide sequence having at least 85%, 90%, 91%, 92%, 93%, 94%, 95%, 96%, 97%, 98%, 99%, or 100% nucleotide identity to the entire nucleotide sequence of GD-28449, and a third nucleotide sequence having at least 85%, 90%, 91%, 92%, 93%, 94%, 95%, 96%, 97%, 98%, 99%, or 100% nucleotide identity to the entire nucleotide sequence of GD-28450.

### BRIEF DESCRIPTION OF THE DRAWINGS

FIGS. 1A and 1B are graphs depicting the activation of FOXP3 expression after contacting Jurkat cells with the indicated pools of site-specific FOXP3 targeting moieties and an effector molecule comprising dCas, dCas9 and p300, or dCas9 and VPR.

FIG. 1A shows qPCR quantification of FOXP3 mRNA levels 48 hour after transfection with either dCas9+sgRNA pools (1, 2, or 3) or with dCas9−p300+sgRNA pools (1, 2, or 3) or with dCas9−VPR+sgRNA pools (1, 2, or 3).

FIG. 1B shows quantitation of a FACS experiment determining the percentage of Jurkat cells that were FOXP3 positive 72 hours post transfection. All transfections were carried out using Lipofectamine MessengerMax reagent (Thermofisher) following the manufacturer's instructions.

FIG. 2 is graph depicting the activation of FOXP3 expression after contacting Jurkat cells with pools of site-specific FOXP3 targeting moieties and an effector molecule comprising dCas9 and p300, or dCas9 and VPR. Only the combination of sgRNA pool 2 and dCas9+VPR showed significantly high FOXP3 activation of both mRNA level and protein level.

FIGS. 3A and 3B are graphs depicting activation of naïve T-cells after contacting the cells with the indicated pools of site-specific FOXP3 targeting moieties and an effector molecule comprising dCas9, dCas9 and p300, or dCas9 and VPR.

FIG. 3A shows qPCR quantification of FOXP3 mRNA levels 58 hours after transfection with either dCas9+sgRNA pool-2 or with dCas9-p300+sgRNA pool-2 or with dCas9-VPR+sgRNA pool-2. FIG. 3B shows quantitation of the FACS experiment determining the percentage of naïve T-cells that were FOXP3 positive 72 hours post transfection. All transfections were carried out using the MaxCyte electroporation buffer and an ATx electroporation system according to the manufacturer's instructions. “Program T cell 2” and “Program T cell 3” are two of the electroporation settings on the instrument used for electroporating the T cells with mRNA+sgRNA for delivery into the cells.

### DETAILED DESCRIPTION OF THE INVENTION

The present invention provides agents and compositions for modulating the expression (e.g., enhanced or reduced expression) of a forkhead box P3 (FOXP3) gene by targeting a FOXP3 expression control region. The FOXP3 gene may be in a cell, e.g., a mammalian cell, such as a mammalian immune cell, e.g., a mammalian naïve T cell, e.g., a human or mouse naïve T cell. The present invention also provides methods of using the agents and compositions of the invention for modulating the expression of a FOXP3 gene in, and/or for treating, a subject who would benefit from modulating the expression of a FOXP3 gene, e.g., a subject suffering or prone to suffering from an autoimmune disease.

The agents of the invention are referenced to herein as site-specific FOXP3 disrupting agents and are described in Section II, below.

### I. Definitions

In order that the present invention may be more readily understood, certain terms are first defined. In addition, it should be noted that whenever a value or range of values of a parameter are recited, it is intended that values and ranges intermediate to the recited values are also intended to be part of this invention.

The articles “a” and “an” are used herein to refer to one or to more than one (i.e., to at least one) of the grammatical object of the article. By way of example, “an element” means one element or more than one element, e.g., a plurality of elements, e.g., a pool of elements, such as sgRNAs.

The term “including” is used herein to mean, and is used interchangeably with, the phrase “including but not limited to”. The term “or” is used herein to mean, and is used interchangeably with, the term “and/or,” unless context clearly indicates otherwise.

The term “about” is used herein to mean within the typical ranges of tolerances in the art. For example, “about” can be understood as about 2 standard deviations from the mean. In certain embodiments, about means±10%. In certain embodiments, about means±5%. When about is present before a series of numbers or a range, it is understood that “about” can modify each of the numbers in the series or range.

The term “at least” prior to a number or series of numbers is understood to include the number adjacent to the term “at least”, and all subsequent numbers or integers that could logically be included, as clear from context. For example, the number of nucleotides in a nucleic acid molecule must be an integer. For example, “at least 18 nucleotides of a 21 nucleotide nucleic acid molecule” means that 18, 19, 20, or 21 nucleotides have the indicated property. When at least is present before a series of numbers or a range, it is understood that “at least” can modify each of the numbers in the series or range.

As used herein, “no more than” or “less than” is understood as the value adjacent to the phrase and logical lower values or integers, as logical from context, to zero. When “no more than” is present before a series of numbers or a range, it is understood that “no more than” can modify each of the numbers in the series or range.

As used herein, the term “substantially” refers to the qualitative condition of exhibiting total or near-total extent or degree of a characteristic or property of interest. One of ordinary skill in the art will understand that biological and chemical phenomena rarely, if ever, go to completion and/or proceed to completeness or achieve or avoid an absolute result. The term “substantially” may therefore be used in some embodiments herein to capture potential lack of completeness inherent in many biological and chemical phenomena.

As used herein, the term “forkhead box P3” or “FOXP3” refers to the gene that encodes the well known FOX protein family member that is a master transcription factor that controls the differentiation of naïve T-cells into regulatory T-cells (Tregs). FOX proteins belong to the forkhead/winged-helix family of transcriptional regulators and are believed to exert control via similar DNA binding interactions during transcription. In regulatory T cell model systems, the FOXP3 transcription factor occupies the promoters for genes involved in regulatory T-cell function, and may repress transcription of key genes following stimulation of T cell receptors. Defects in this gene's ability to function can cause immunodysregulation polyendocrinopathy enteropathy X-linked syndrome (or IPEX), also known as X-linked autoimmunity-immunodeficiency syndrome, as well as numerous cancers. The nucleotide and amino acid sequence of FOXP3 is known and may be found in, for example, GenBank Accession Nos. NM_014009.4 and NM_001114377.2, the entire contents of each of which are incorporated herein by reference. The nucleotide sequence of the genomic region of the X Chromosome in human, which includes the endogenous promoters of FOXP3 and the FOXP3 coding sequence, is also known and may be found in, for example, NC_000023.11 (49250436-49264932) (SEQ ID NO:5). There are two common transcript variants for FOXP3 mRNA, the sequences of which can be found in GenBank Accession Nos. NM_014009.4 (SEQ ID NO:3) and NM_001114377.2 (SEQ ID NO:4). The entire contents of each of the foregoing GenBank Accession numbers are incorporated herein by reference as of the date of filing this application.

The term “site-specific FOXP3 disrupting agent,” as used herein, refers to any agent that specifically binds to a target FOXP3 expression control region and, e.g., modulates expression of a FOXP3 gene. Site-specific FOXP3 disruption agents of the invention may comprise a “site-specific FOXP3 targeting moiety.”

As used herein, the term “site-specific FOXP3 targeting moiety” refers to a moiety that specifically binds to a FOXP3 expression control region, e.g., a transcriptional control region of a FOXP3 gene, such as a DNA region around/proximally upstream of the transcription start site, a promoter, an enhancer, or a repressor; or a FOXP3-associated anchor sequence, such as, for example within a FOXP3-associated anchor sequence-mediated conjunction. Exemplary “site-specific FOXP3 targeting moieties” include, but are not limited to, polyamides, nucleic acid molecules, such as RNA, DNA, or modified RNA or DNA, polypeptides, protein nucleic acid molecules, and fusion proteins.

As used herein, the terms “specific binding” or “specifically binds” refer to an ability to discriminate between possible binding partners in the environment in which binding is to occur. In some embodiments, a disrupting agent that interacts, e.g., preferentially interacts, with one particular target when other potential disrupting agents are present is said to “bind specifically” to the target (i.e., the expression control region) with which it interacts. In some embodiments, specific binding is assessed by detecting or determining the degree of association between the disrupting agent and its target; in some embodiments, specific binding is assessed by detecting or determining degree of dissociation of a disrupting agent-target complex. In some embodiments, specific binding is assessed by detecting or determining ability of the disrupting agent to compete with an alternative interaction between its target and another entity. In some embodiments, specific binding is assessed by performing such detections or determinations across a range of concentrations.

As used herein, the term “expression control region” or expression control domain′ refers to a region or domain present in a genomic DNA that modulates the expression of a target gene in a cell. A functionality associated with an expression control region may directly affect expression of a target gene, e.g., by recruiting or blocking recruitment of a transcription factor that would stimulate expression of the gene. A functionality associated with an expression control region may indirectly affect expression of a target gene, e.g., by introducing epigenetic modifications or recruiting other factors that introduce epigenetic modifications that induce a change in chromosomal topology that modulates expression of a target gene. Expression control regions may be upstream and/or downstream of the protein coding sequence of a gene and include, for example, transcriptional control elements, e.g., DNA regions around/proximally upstream of the transcription start site, promoters, enhancers, or repressors; and anchor sequences, and anchor sequence-mediated conjunctions.

The term “transcriptional control element,” as used herein, refers to a nucleic acid sequence that controls transcription of a gene. Transcriptional control elements include, for example, anchor sequences, anchor sequence-mediated conjunctions, DNA regions around/proximally upstream of the transcription start site, promoters, transcriptional enhancers, and transcriptional repressors.

A transcription start site (TSS) is the location where transcription starts at the 5′-end of a gene sequence. The DNA regions around/proximally upstream of the TSS can regulate the expression of a gene by, for example, recruiting a transcription factor. Alteration in the modification status of one or more nucleotides (e.g., methylation) or one or more chromatin proteins (e.g., acetylation) in the DNA regions around/proximally upstream of the TSS can regulate the expression of a gene.

A promoter is a region of DNA recognized by an RNA polymerase to initiate transcription of a particular gene and is generally located upstream of the 5′-end of the transcription start site of the gene.

A “transcriptional enhancer” increases gene transcription. A “transcriptional silencer” or “transcriptional repressor” decreases gene transcription. Enhancing and silencing sequences may be about 50-3500 base pairs in length and may influence gene transcription up to about 1 megabases away.

The term “gene,” as used herein, refers to a sequence of nucleotides that encode a molecule, such as a protein, that has a function. A gene contains sequences that are transcribed (e.g., a 3′UTR), sequences that are not transcribed (e.g., a promoter), sequences that are translated (e.g., an exon), and sequences that are not translated (e.g., intron).

As used herein, the term “target gene” means a FOXP3 gene that is targeted for modulation, e.g., increase or decrease, of expression. In some embodiments, a FOXP3 target gene is part of a targeted genomic complex (e.g. a FOXP3 gene that has at least part of its genomic sequence as part of a target genomic complex, e.g. inside an anchor sequence-mediated conjunction), which genomic complex is targeted by one or more site-specific disrupting agents as described herein. In some embodiments, modulation comprises activation of expression of the target gene. In some embodiments, a FOXP3 gene is modulated by contacting the FOXP3 gene or a transcription control element operably linked to the FOXP3 gene with one or more site-specific disrupting agents as described herein. In some embodiments, a FOXP3 gene is aberrantly expressed (e.g., over-expressed) in a cell, e.g., a cell in a subject (e.g., a subject having a FOXP3-associated disease or an autoimmune disease). In some embodiments, a FOXP3 gene is aberrantly expressed (e.g., under-expressed) in a cell, e.g., a cell in a subject (e.g., a subject having a FOXP3-associated disease or an autoimmune disease).

The term “anchor sequence” as used herein, refers to a nucleic acid sequence recognized by a nucleating agent that binds sufficiently to form an anchor sequence-mediated conjunction, e.g., a complex. In some embodiments, an anchor sequence comprises one or more CTCF binding motifs. In some embodiments, an anchor sequence is not located within a gene coding region. In some embodiments, an anchor sequence is located within an intergenic region. In some embodiments, an anchor sequence is not located within either of an enhancer or a promoter. In some embodiments, an anchor sequence is located at least 400 bp, at least 450 bp, at least 500 bp, at least 550 bp, at least 600 bp, at least 650 bp, at least 700 bp, at least 750 bp, at least 800 bp, at least 850 bp, at least 900 bp, at least 950 bp, or at least 1 kb away from any transcription start site. In some embodiments, an anchor sequence is located within a region that is not associated with genomic imprinting, monoallelic expression, and/or monoallelic epigenetic marks. In some embodiments, the anchor sequence has one or more functions selected from binding an endogenous nucleating polypeptide (e.g., CTCF), interacting with a second anchor sequence to form an anchor sequence mediated conjunction, or insulating against an enhancer that is outside the anchor sequence mediated conjunction. In some embodiments of the present invention, technologies are provided that may specifically target a particular anchor sequence or anchor sequences, without targeting other anchor sequences (e.g., sequences that may contain a nucleating agent (e.g., CTCF) binding motif in a different context); such targeted anchor sequences may be referred to as the “target anchor sequence”. In some embodiments, sequence and/or activity of a target anchor sequence is modulated while sequence and/or activity of one or more other anchor sequences that may be present in the same system (e.g., in the same cell and/or in some embodiments on the same nucleic acid molecule, e.g., the same chromosome) as the other targeted anchor sequence is not modulated. In some embodiments, the anchor sequence comprises or is a nucleating polypeptide binding motif. In some embodiments, the anchor sequence is adjacent to a nucleating polypeptide binding motif.

The term “anchor sequence-mediated conjunction” as used herein, refers to a DNA structure, in some cases, a complex, that occurs and/or is maintained via physical interaction or binding of at least two anchor sequences in the DNA by one or more polypeptides, such as nucleating polypeptides, or one or more proteins and/or a nucleic acid entity (such as RNA or DNA), that bind the anchor sequences to enable spatial proximity and functional linkage between the anchor sequences.

As used herein, the term “genomic complex” is a complex that brings together two genomic sequence elements that are spaced apart from one another on one or more chromosomes, via interactions between and among a plurality of protein and/or other components (potentially including, the genomic sequence elements). In some embodiments, the genomic sequence elements are anchor sequences to which one or more protein components of the complex bind. In some embodiments, a genomic complex may comprise an anchor sequence-mediated conjunction. In some embodiments, a genomic sequence element may be or comprise a CTCF binding motif, a promoter and/or an enhancer. In some embodiments, a genomic sequence element includes at least one or both of a promoter and/or regulatory region (e.g., an enhancer). In some embodiments, complex formation is nucleated at the genomic sequence element(s) and/or by binding of one or more of the protein component(s) to the genomic sequence element(s). As will be understood by those skilled in the art, in some embodiments, co-localization (e.g., conjunction) of the genomic sites via formation of the complex alters DNA topology at or near the genomic sequence element(s), including, in some embodiments, between them. In some embodiments, a genomic complex comprises an anchor sequence-mediated conjunction, which comprises one or more loops. In some embodiments, a genomic complex as described herein is nucleated by a nucleating polypeptide such as, for example, CTCF and/or Cohesin. In some embodiments, a genomic complex as described herein may include, for example, one or more of CTCF, Cohesin, non-coding RNA (e.g., eRNA), transcriptional machinery proteins (e.g., RNA polymerase, one or more transcription factors, for example selected from the group consisting of TFIIA, TFIIB, TFIID, TFIIE, TFIIF, TFIIH, etc.), transcriptional regulators (e.g., Mediator, P300, enhancer-binding proteins, repressor-binding proteins, histone modifiers, etc.), etc. In some embodiments, a genomic complex as described herein includes one or more polypeptide components and/or one or more nucleic acid components (e.g., one or more RNA components), which may, in some embodiments, be interacting with one another and/or with one or more genomic sequence elements (e.g., anchor sequences, promoter sequences, regulatory sequences (e.g., enhancer sequences)) so as to constrain a stretch of genomic DNA into a topological configuration (e.g., a loop) that the stretch of genomic DNA does not adopt when the complex is not formed.

An “effector molecule,” as used herein, refers to a molecule that is able to regulate a biological activity, such as enzymatic activity, gene expression, anchor sequence-mediated conjunction or cell signaling. Exemplary effectors are described in Section II, below, and in some embodiment include, for example, nucleases, physical blockers, epigenetic recruiters, e.g., a transcriptional enhancer or a transcriptional repressor, and epigenetic CpG modifiers, e.g., a DNA methylase, a DNA demethylase, a histone modifying agent, a histone transacetylase, or a histone deacetylase, and combinations of any of the foregoing.

### II. Site-Specific FOXP3 Disrupting Agents of the Invention

The present invention provides site-specific FOXP3 disrupting agents which, in one aspect of the invention, include a site-specific FOXP3 targeting moiety which targets a FOXP3 expression control region. In another aspect, the site-specific disrupting agents of the invention include a site-specific FOXP3 targeting moiety which targets a FOXP3 expression control region and an effector molecule. As will be appreciated by one of ordinary skill in the art, such disrupting agents are site-specific and, thus, specifically bind to a FOXP3 expression control region (e.g., one or more transcriptional control elements and/or one or more target anchor sequences), e.g., within a cell, and not to non-targeted expression control regions (e.g., within the same cell).

FOXP3 is a master transcription factor that controls the differentiation of naïve T-cells into regulatory T-cells (Tregs) and forced overexpression of FOXP3 has been shown to confer the Treg phenotype to T-cells. The present invention features the use of effector molecules, e.g., chromatin remodelers, that when fused to DNA-targeting moieties can induce epigenetic changes at specific genomic regions that lead to increased transcription of targeted genes, e.g., FOXP3 gene. In certain embodiments, an effector molecule, p300-core or VPR, fused to dCas9, which is the DNA targeting moiety, is targeted to the FOXP3 locus using single guide RNAs (sgRNAs) complementary to the DNA region around/just upstream of the transcription start site (TSS) of a FOXP3 gene and provokes changes in histone acetylation. These epigenetic changes trigger mechanisms which ultimately result in activating FOXP3 in naïve T-cells and inducing differentiation to Tregs. These Tregs can be identified based on cell surface markers, such as CD127, and/or based on a suppression phenotype where Tregs kill effector T-cells incubated in a mixed culture.

In vitro generation of Tregs has been an important effort in the field of ex vivo therapy targeting auto-immune disorders. However, many of the strategies to produce Tregs either do not lead to sustained expression of genes that lead to Tregs or give Tregs that have a suppression phenotype. The present invention features methods to directly target the master regulator transcription factor in the Treg generation and maintenance pathway, FOXP3, using targeting moieties (e.g., dCas9, TALEs or ZFP) to directly deliver an effector molecule (e.g., an activator) to the site of action to increase activation of FOXP3 gene.

The site-specific FOXP3 disrupting agents of the invention comprise a site-specific FOXP3 targeting moiety targeting a FOXP3 expression control region. The expression control region targeted by the site-specific targeting moiety may be, for example, a transcriptional control element or an anchor sequence, such as an anchor sequence within an anchor-mediated conjunction.

Thus, site-specific FOXP3 disrupting agents of the invention may modulate expression of a gene, i.e., FOXP3, e.g., by modulating expression of the gene from a DNA region around/proximally upstream of a transcription start site, an endogenous promoter, an enhancer, or an repressor; may alter methylation of the control region; may alter acetylation of the chromatin protein, may introduce one or more mutations, e.g., substitution, addition or deletion of nucleotide; may alter at least one anchor sequence; may alter at least one conjunction nucleating molecule binding site, such as by altering binding affinity for the conjunction nucleating molecule; may alter an orientation of at least one common nucleotide sequence, such as a CTCF binding motif by, e.g., substitution, addition or deletion in at least one anchor sequence, such as a CTCF binding motif.

In certain embodiments, the site-specific disrupting agents and compositions described herein target an expression control region comprising one or more FOXP3-specific transcriptional control elements to modulate expression in a cell. FOXP3-specific transcriptional control elements that can be targeted include DNA region around or proximally upstream of FOXP3-transcription start site, FOXP3-specific promoter, FOXP3-specific enhancers, FOXP3-specific repressors, and FOXP3-associated anchor sequence. In one embodiment, FOXP3-specific transcriptional control element regulates expression in immune cells, e.g., DNA region around or proximally upstream of FOXP3-transcription start site.

For example, a site-specific disrupting agent may include a site-specific targeting moiety, e.g., a nucleic acid molecule encoding a DNA-binding domain of a Transcription activator-like effector (TALE) polypeptide or a zinc finger (ZNF) polypeptide, or fragment thereof, that specifically targets and binds to a FOXP3 expression control region, such as a FOXP3 endogenous promoter region, and an effector molecule, such as an effector molecule that includes a transcriptional enhancer or transcriptional repressor that modulates, e.g., enhances or represses, expression of a target gene from an endogenous promoter to modulate gene expression. In one embodiment, the disrupting agent is “bicistronic nucleic acid molecule,” i.e., capable of making two fusion proteins from a single messenger RNA molecule, a first and a second site-specific targeting moiety, e.g., a nucleic acid molecule encoding a DNA-binding domain of a Transcription activator-like effector (TALE) polypeptide or a zinc finger (ZNF) polypeptide, or fragment thereof, that specifically targets and binds to a FOXP3 expression control region, such as a FOXP3 endogenous promoter region, and an effector molecule, such as an effector molecule that includes a transcriptional enhancer or transcriptional repressor that modulates, e.g., enhances or represses, expression of a target gene from an endogenous promoter to modulate gene expression.

In some embodiments of the invention, a site-specific disrupting agent may include a site-specific targeting moiety, e.g., a nucleic acid molecule, such as a guide RNA targeting a FOXP3 endogenous DNA region around or proximally upstream of FOXP3 transcription starting site, and an effector molecule, such as an effector molecule that includes a transcriptional enhancer or transcriptional repressor that modulates, e.g., enhances or represses, expression of a target gene from an endogenous promoter to modulate gene expression.

In certain embodiments of the invention, the site-specific disrupting agents and compositions described herein target an expression control region comprising one or more FOXP3-associated anchor sequences, e.g., within an anchor sequence-mediated conjunction, comprising a first and a second FOXP3-associated anchor sequence to alter a two-dimensional chromatin structure, e.g., anchor sequence-mediated conjunctions in order to modulate expression in a cell, e.g., a cell within a subject, e.g., by modifying anchor sequence-mediated conjunctions in DNA, e.g., genomic DNA.

In one aspect, the invention includes a site-specific FOXP3 disrupting agent comprising a site-specific FOXP3 targeting moiety which targets a FOXP3 expression control region comprising one or more FOXP3-associated anchor sequences within an anchor sequence-mediated conjunction. The disrupting agent binds, e.g., specifically binds, a specific anchor sequence-mediated conjunction to alter a topology of the anchor sequence-mediated conjunction, e.g., an anchor sequence-mediated conjunction having a physical interaction of two or more DNA loci bound by a conjunction nucleating molecule.

The formation of an anchor sequence-mediated conjunction may force transcriptional control elements to interact with a FOXP3 gene or spatially constrain the activity of the transcriptional control elements. Altering anchor sequence-mediated conjunctions, therefore, allows for modulating FOXP3 expression without altering the coding sequences of the FOXP3 gene being modulated.

In some embodiments, the site-specific disrupting agents and compositions of the invention modulate expression of a FOXP3 gene associated with an anchor sequence-mediated conjunction by physically interfering between one or more anchor sequences and a conjunction nucleating molecule. For example, a DNA binding small molecule (e.g., minor or major groove binders), peptide (e.g., zinc finger, TALE, novel or modified peptide), protein (e.g., CTCF, modified CTCF with impaired CTCF binding and/or cohesion binding affinity), or nucleic acids (e.g., ssDNA, modified DNA or RNA, peptide oligonucleotide conjugates, locked nucleic acids, bridged nucleic acids, polyamides, and/or triplex forming oligonucleotides) may physically prevent a conjunction nucleating molecule from interacting with one or more anchor sequences to modulate FOXP3 gene expression.

In some embodiments, the site-specific disrupting agents and compositions of the invention modulate expression of a FOXP3 gene associated with an anchor sequence-mediated conjunction by modification of an anchor sequence, e.g., epigenetic modifications, e.g., histone protein modifications, or genomic editing modifications. For example, one or more anchor sequences associated with an anchor sequence-mediated conjunction comprising a FOXP3 gene may be targeted for genome editing, e.g., Cas9-mediated genome editing.

In some embodiments, the site-specific disrupting agents and compositions of the invention modulate expression of a FOXP3 gene associated with an anchor sequence-mediated conjunction, e.g., activate or represses transcription, e.g., induces epigenetic changes to chromatin or genome editing.

In some embodiments, an anchor sequence-mediated conjunction includes one or more anchor sequences, a FOXP3 gene, and one or more transcriptional control elements, such as an enhancing or silencing element. In some embodiments, the transcriptional control element is within, partially within, or outside the anchor sequence-mediated conjunction.

In one embodiment, the anchor sequence-mediated conjunction comprises a loop, such as an intra-chromosomal loop. In certain embodiments, the anchor sequence-mediated conjunction has a plurality of loops. One or more loops may include a first anchor sequence, a nucleic acid sequence, a transcriptional control element, and a second anchor sequence. In another embodiment, at least one loop includes, in order, a first anchor sequence, a transcriptional control element, and a second anchor sequence; or a first anchor sequence, a nucleic acid sequence, and a second anchor sequence. In yet another embodiment, either one or both of the nucleic acid sequences and the transcriptional control element is located within or outside the loop. In still another embodiment, one or more of the loops comprises a transcriptional control element.

In some embodiments, the anchor sequence-mediated conjunction includes a TATA box, a CAAT box, a GC box, or a CAP site.

In some embodiments, the anchor sequence-mediated conjunction comprises a plurality of loops, and where the anchor sequence-mediated conjunction comprises at least one of an anchor sequence, a nucleic acid sequence, and a transcriptional control element in one or more of the loops.

In one aspect, the site-specific disrupting agents and compositions of the invention may introduce a targeted alteration to an anchor sequence-mediated conjunction to modulate expression of a nucleic acid sequence with a disrupting agent that binds the anchor sequence. In some embodiments, the anchor sequence-mediated conjunction is altered by targeting one or more nucleotides within the anchor sequence-mediated conjunction for substitution, addition or deletion.

In some embodiments, expression, e.g., transcription, is activated by inclusion of an activating loop or exclusion of a repressive loop. In one such embodiment, the anchor sequence-mediated conjunction comprises a transcriptional control sequence that increases transcription of a nucleic acid sequence, e.g., such a FOXP3 encoding nucleic acid. In another such embodiment, the anchor sequence-mediated conjunction excludes a transcriptional control element that decreases expression, e.g., transcription, of a nucleic acid sequence, e.g., such a FOXP3 encoding nucleic acid.

In some embodiments, expression, e.g., transcription, is repressed by inclusion of a repressive loop or exclusion of an activating loop. In one such embodiment, the anchor sequence-mediated conjunction includes a transcriptional control element that decreases expression, e.g., transcription, of a nucleic acid sequence, e.g., such a FOXP3 encoding nucleic acid sequence. In another such embodiment, the anchor sequence-mediated conjunction excludes a transcriptional control sequence that increases transcription of a nucleic acid sequence, e.g., such a FOXP3 encoding nucleic acid.

Each anchor sequence-mediated conjunction comprises one or more anchor sequences, e.g., a plurality. Anchor sequences can be manipulated or altered to disrupt naturally occurring loops or form new loops (e.g., to form exogenous loops or to form non-naturally occurring loops with exogenous or altered anchor sequences). Such alterations modulate FOXP3 gene expression by changing the 2-dimensional structure of DNA containing all or a portion of a FOXP3 gene, e.g., by thereby modulating the ability of the FOXP3 gene to interact with transcriptional control elements (e.g., enhancing and silencing/repressive sequences). In some embodiments, the chromatin structure is modified by substituting, adding or deleting one or more nucleotides within an anchor sequence of the anchor sequence-mediated conjunction.

The anchor sequences may be non-contiguous with one another. In embodiments with noncontiguous anchor sequences, the first anchor sequence may be separated from the second anchor sequence by about 500 bp to about 500 Mb, about 750 bp to about 200 Mb, about 1 kb to about 100 Mb, about 25 kb to about 50 Mb, about 50 kb to about 1 Mb, about 100 kb to about 750 kb, about 150 kb to about 500 kb, or about 175 kb to about 500 kb. In some embodiments, the first anchor sequence is separated from the second anchor sequence by about 500 bp, 600 bp, 700 bp, 800 bp, 900 bp, 1 kb, 5 kb, 10 kb, 15 kb, 20 kb, 25 kb, 30 kb, 35 kb, 40 kb, 45 kb, 50 kb, 55 kb, 60 kb, 65 kb, 70 kb, 75 kb, 80 kb, 85 kb, 90 kb, 95 kb, 100 kb, 125 kb, 150 kb, 175 kb, 200 kb, 225 kb, 250 kb, 275 kb, 300 kb, 350 kb, 400 kb, 500 kb, 600 kb, 700 kb, 800 kb, 900 kb, 1 Mb, 2 Mb, 3 Mb, 4 Mb, 5 Mb, 6 Mb, 7 Mb, 8 Mb, 9 Mb, 10 Mb, 15 Mb, 20 Mb, 25 Mb, 50 Mb, 75 Mb, 100 Mb, 200 Mb, 300 Mb, 400 Mb, 500 Mb, or any size therebetween.

In one embodiment, the anchor sequence comprises a common nucleotide sequence, e.g., a CTCF-binding motif:

(SEQ ID NO: 1)
N(T/C/G)N(G/A/T)CC(A/T/G)(C/G)(C/T/A)AG(G/A)(G/T)
GG(C/A/T)(G/A)(C/G)(C/T/A)(G/A/C),

where N is any nucleotide.

A CTCF-binding motif may also be in the opposite orientation, e.g.,

(SEQ ID NO: 2)
(G/A/C)(C/T/A)(C/G)(G/A)(C/A/T)GG(G/T)(G/A)GA
(C/T/A)(C/G)(A/T/G)CC(G/A/T)N(T/C/G)N .

In one embodiment, the anchor sequence comprises SEQ ID NO: 1 or SEQ ID NO:2 or a nucleotide sequence at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical to either SEQ ID NO: 1 or SEQ ID NO:2.

In some embodiments, the anchor sequence-mediated conjunction comprises at least a first anchor sequence and a second anchor sequence. The first anchor sequence and second anchor sequence may each comprise a common nucleotide sequence, e.g., each comprises a CTCF binding motif. In some embodiments, the first anchor sequence and second anchor sequence comprise different sequences, e.g., the first anchor sequence comprises a CTCF binding motif and the second anchor sequence comprises an anchor sequence other than a CTCF binding motif. In some embodiments, each anchor sequence comprises a common nucleotide sequence and one or more flanking nucleotides on one or both sides of the common nucleotide sequence.

Two CTCF-binding motifs (e.g., contiguous or non-contiguous CTCF binding motifs) that can form a conjunction may be present in the genome in any orientation, e.g., in the same orientation (tandem) either 5′->3′ (left tandem, e.g., the two CTCF-binding motifs that comprise SEQ ID NO: 1) or 3′->5′ (right tandem, e.g., the two CTCF-binding motifs comprise SEQ ID NO:2), or convergent orientation, where one CTCF-binding motif comprises SEQ ID NO: 1 and the other comprises SEQ ID NO:2. CTCFBSDB 2.0: Database For CTCF binding motifs And Genome Organization (http://insulatordb.uthsc.edu/) can be used to identify CTCF binding motifs associated with a target gene, e.g., FOXP3.

In some embodiments, the anchor sequence-mediated conjunction is altered by changing an orientation of at least one common nucleotide sequence, e.g., a conjunction nucleating molecule binding site.

In some embodiments, the anchor sequence comprises a conjunction nucleating molecule binding site, e.g., CTCF binding motif, and site-specific disrupting agent of the invention introduces an alteration in at least one conjunction nucleating molecule binding site, e.g. altering binding affinity for the conjunction nucleating molecule.

In some embodiments, the anchor sequence-mediated conjunction is altered by introducing an exogenous anchor sequence. Addition of a non-naturally occurring or exogenous anchor sequence to form or disrupt a naturally occurring anchor sequence-mediated conjunction, e.g., by inducing a non-naturally occurring loop to form that alters transcription of the nucleic acid sequence.

In some embodiments, the anchor sequence-mediated conjunction comprises a FOXP3 gene, and one or more, e.g., 2, 3, 4, 5, or other genes other than the FOXP3 gene.

In some embodiments, the anchor sequence-mediated conjunction is associated with one or more, e.g., 2, 3, 4, 5, or more, transcriptional control elements. In some embodiments, the FOXP3 gene is noncontiguous with one or more of the transcriptional control elements. In some embodiments where the FOXP3 gene is non-contiguous with the transcriptional control element, the gene may be separated from one or more transcriptional control elements by about 100 bp to about 500 Mb, about 500 bp to about 200 Mb, about 1 kb to about 100 Mb, about 25 kb to about 50 Mb, about 50 kb to about 1 Mb, about 100 kb to about 750 kb, about 150 kb to about 500 kb, or about 175 kb to about 500 kb. In some embodiments, the gene is separated from the transcriptional control element by about 100 bp, 300 bp, 500 bp, 600 bp, 700 bp, 800 bp, 900 bp, 1 kb, 5 kb, 10 kb, 15 kb, 20 kb, 25 kb, 30 kb, 35 kb, 40 kb, 45 kb, 50 kb, 55 kb, 60 kb, 65 kb, 70 kb, 75 kb, 80 kb, 85 kb, 90 kb, 95 kb, 100 kb, 125 kb, 150 kb, 175 kb, 200 kb, 225 kb, 250 kb, 275 kb, 300 kb, 350 kb, 400 kb, 500 kb, 600 kb, 700 kb, 800 kb, 900 kb, 1 Mb, 2 Mb, 3 Mb, 4 Mb, 5 Mb, 6 Mb, 7 Mb, 8 Mb, 9 Mb, 10 Mb, 15 Mb, 20 Mb, 25 Mb, 50 Mb, 75 Mb, 100 Mb, 200 Mb, 300 Mb, 400 Mb, 500 Mb, or any size therebetween.

In some embodiments, the type of anchor sequence-mediated conjunction may help to determine how to modulate gene expression, e.g., choice of site-specific targeting moiety, by altering the anchor sequence-mediated conjunction. For example, some types of anchor sequence-mediated conjunctions comprise one or more transcription control elements within the anchor sequence-mediated conjunction. Disruption of such an anchor sequence-mediated conjunction by disrupting the formation of the anchor sequence-mediated conjunction, e.g., altering one or more anchor sequences, is likely to decrease transcription of a FOXP3 gene within the anchor sequence-mediated conjunction.

In some embodiments, expression of the FOXP3 gene is regulated, modulated, or influenced by one or more transcriptional control elements associated with the anchor sequence-mediated conjunction. In some embodiments, the anchor sequence-mediated conjunction comprises a FOXP3 gene and one or more transcriptional control elements. For example, the FOXP3 gene and one or more transcriptional control sequences are located within, at least partially, an anchor sequence-mediated conjunction, e.g., a Type 1 anchor sequence-mediated conjunction. The anchor sequence-mediated conjunction may also be referred to as a “Type 1, EP subtype.” In some embodiments, the FOXP3 gene has a defined state of expression, e.g., in its native state, e.g., in a diseased state. For example, the FOXP3 gene may have a high level of expression. By disrupting the anchor sequence-mediated conjunction, expression of the FOXP3 gene may be decreased, e.g., decreased transcription due to conformational changes of the DNA previously open to transcription within the anchor sequence-mediated conjunction, e.g., decreased transcription due to conformational changes of the DNA creating additional distance between the FOXP3 gene and the enhancing sequences. In one embodiment, both the FOXP3 gene associated and one or more transcriptional control sequences, e.g., enhancing sequences, reside inside the anchor sequence-mediated conjunction. Disruption of the anchor sequence-mediated conjunction decreases expression of the FOXP3 gene. In one embodiment, the FOXP3 gene associated with the anchor sequence-mediated conjunction is accessible to one or more transcriptional control elements that reside inside, at least partially, the anchor sequence-mediated conjunction.

In some embodiments, expression of the FOXP3 gene is regulated, modulated, or influenced by one or more transcriptional control elements associated with, but inaccessible due to the anchor sequence-mediated conjunction. For example, the anchor sequence-mediated conjunction associated with a FOXP3 gene disrupts the ability of one or more transcriptional control elements to regulate, modulate, or influence expression of the FOXP3 gene. The transcriptional control sequences may be separated from the FOXP3 gene, e.g., reside on the opposite side, at least partially, e.g., inside or outside, of the anchor sequence-mediated conjunction as the FOXP3 gene, e.g., the FOXP3 gene is inaccessible to the transcriptional control elements due to proximity of the anchor sequence-mediated conjunction. In some embodiments, one or more enhancing sequences are separated from the FOXP3 gene by the anchor sequence-mediated conjunction, e.g., a Type 2 anchor sequence-mediated conjunction.

In some embodiments, the FOXP3 gene is inaccessible to one or more transcriptional control elements due to the anchor sequence-mediated conjunction, and disruption of the anchor sequence-mediated conjunction allows the transcriptional control element to regulate, modulate, or influence expression of the FOXP3 gene. In one embodiment, the FOXP3 gene is inside and outside the anchor sequence-mediated conjunction and inaccessible to the one or more transcriptional control elements. Disruption of the anchor sequence-mediated conjunction increases access of the transcriptional control elements to regulate, modulate, or influence expression of the FOXP3 gene, e.g., the transcriptional control elements increase expression of the FOXP3 gene. In one embodiment, the FOXP3 gene is inside the anchor sequence-mediated conjunction and inaccessible to the one or more transcriptional control elements residing outside, at least partially, the anchor sequence-mediated conjunction. Disruption of the anchor sequence-mediated conjunction increases expression of the FOXP3 gene. In one embodiment, the FOXP3 gene is outside, at least partially, the anchor sequence-mediated conjunction and inaccessible to the one or more transcriptional control elements residing inside the anchor sequence-mediated conjunction. Disruption of the anchor sequence-mediated conjunction increases expression of the FOXP3 gene.

A. FOXP3 Site-Specific Targeting Moieties

The site-specific FOXP3 targeting moieties of the invention target a FOXP3 expression control region and may comprise a polymer or polymeric molecule, such as a polyamide (i.e., a molecule of repeating units linked by amide binds, e.g., a polypeptide), a polymer of nucleotides (such as a guide RNA, a nucleic acid molecule encoding a TALE polypeptide or a zinc finger polypeptides), a peptide nucleic acid (PNA), or a polymer of amino acids, such as a peptide or polypeptide, e.g., a fusion protein, etc. Suitable site-specific FOXP3 targeting moieties, compositions, and methods of use of such agents and compositions are described below and in PCT Publication WO 2018/049073, the entire contents of which are expressly incorporated herein by reference.

In one embodiment, a site-specific disrupting agent of the invention comprises a site-specific FOXP3 targeting moiety comprising a nucleic acid molecule, such as a guide RNA (or gRNA) or a guide RNA and an effector, or fragment thereof, or nucleic acid molecule encoding an effector, or fragment thereof.

In another embodiment, a site specific disrupting agent of the invention comprises a site-specific FOXP3 targeting moiety comprising a nucleic acid molecule encoding a polypeptide, such as a DNA-binding domain, or fragment thereof, of a zinc finger polypeptide (ZNF) or a transcription activator-like effector (i.e., a TALE DNA binding domain, or TALE) polypeptide, that is engineered to specifically target a FOXP3 expression control region to modulate expression of a FOXP3 gene.

In another embodiment, a site-specific disrupting agent of the invention comprises a site-specific FOXP3 targeting moiety comprising a polynucleotide, such as a PNA, e.g., a nucleic acid gRNA linked to an effector polypeptide, or fragment thereof.

In another embodiment, a site-specific disrupting agent of the invention comprises a site-specific FOXP3 targeting moiety comprising a fusion molecule, such as a nucleic acid molecule encoding a DNA-binding domain, of a Transcription activator-like effector (TALE) polypeptide or a zinc finger (ZNF) polypeptide, or fragment thereof, and an effector.

In one embodiment, such site-specific disrupting agents comprise a second fusion protein, wherein the second fusion protein comprises a second site-specific FOXP3 targeting moiety which targets a second FOXP3 expression control region and a second effector molecule, wherein the second FOXP3 expression control region is different than the FOXP3 expression control region.

In another embodiment, a site-specific disrupting agent of the invention comprises a site-specific FOXP3 targeting moiety comprising a fusion molecule, such as a nucleic acid molecule encoding a fusion protein comprising a Cas polypeptide and, e.g., an epigenetic recruiter or an epigenetic CpG modifier.

In yet, another embodiment, a site-specific disrupting agent of the invention comprises a site-specific FOXP3 targeting moiety comprising a fusion molecule, such as fusion protein comprising a Cas polypeptide and, e.g., an epigenetic recruiter or an epigenetic CpG modifier.

As used herein, in its broadest sense, the term “nucleic acid” refers to any compound and/or substance that is or can be incorporated into an oligonucleotide chain. In some embodiments, a nucleic acid is a compound and/or substance that is or can be incorporated into a polynucleotide chain via a phosphodiester linkage. As will be clear from context, in some embodiments, “nucleic acid” refers to an individual nucleic acid residue (e.g., a nucleotide and/or nucleoside); in some embodiments, “nucleic acid” refers to a polynucleotide chain comprising individual nucleic acid residues. In some embodiments, a “nucleic acid” is or comprises RNA; in some embodiments, a “nucleic acid” is or comprises DNA. In some embodiments, a “nucleic acid” is a “mixmer” comprising locked nucleic acid molecules and deoxynucleic acid molecules. In some embodiments, a nucleic acid is, comprises, or consists of one or more natural nucleic acid residues. In some embodiments, a nucleic acid is, comprises, or consists of one or more nucleic acid analogs. In some embodiments, a nucleic acid analog differs from a nucleic acid in that it does not utilize a phosphodiester backbone. For example, in some embodiments, a nucleic acid is, comprises, or consists of one or more “peptide nucleic acids”, which are known in the art and have peptide bonds instead of phosphodiester bonds in the backbone, are considered within the scope of the present invention. Alternatively or additionally, in some embodiments, a nucleic acid has one or more phosphorothioate and/or 5′-N-phosphoramidite linkages rather than phosphodiester bonds. In some embodiments, a nucleic acid is, comprises, or consists of one or more natural nucleosides (e.g., adenosine, thymidine, guanosine, cytidine, uridine, deoxyadenosine, deoxythymidine, deoxy guanosine, and deoxycytidine). In some embodiments, a nucleic acid is, comprises, or consists of one or more nucleoside analogs (e.g., 2-aminoadenosine, 2-thiothymidine, inosine, pyrrolo-pyrimidine, 3-methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, 0(6)-methylguanine, 2-thiocytidine, methylated bases, intercalated bases, and combinations thereof). In some embodiments, a nucleic acid comprises one or more modified sugars (e.g., 2′-fluororibose, ribose, 2′-deoxyribose, arabinose, and hexose) as compared with those in natural nucleic acids. In some embodiments, a nucleic acid has a nucleotide sequence that encodes a functional gene product such as an RNA or protein. In some embodiments, a nucleic acid includes one or more introns. In some embodiments, nucleic acids are prepared by one or more of isolation from a natural source, enzymatic synthesis by polymerization based on a complementary template (in vivo or in vitro), reproduction in a recombinant cell or system, and chemical synthesis. In some embodiments, a nucleic acid is at least 3, 4, 5, 6, 7, 8, 9, 10, 15, 20, 25, 30, 35, 40, 45, 50, 55, 60, 65, 70, 75, 80, 85, 90, 95, 100, 110, 120, 130, 140, 150, 160, 170, 180, 190, 20, 225, 250, 275, 300, 325, 350, 375, 400, 425, 450, 475, 500, 600, 700, 800, 900, 1000, 1500, 2000, 2500, 3000, 3500, 4000, 4500, 5000 or more residues long. In some embodiments, a nucleic acid is partly or wholly single stranded; in some embodiments, a nucleic acid is partly or wholly double stranded. In some embodiments a nucleic acid has a nucleotide sequence comprising at least one element that encodes, or is the complement of a sequence that encodes, a polypeptide. In some embodiments, a nucleic acid has enzymatic activity.

As used herein, the terms “peptide,” “polypeptide,” and “protein” refer to a compound comprised of amino acid residues covalently linked by peptide bonds, or by means other than peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds or by means other than peptide bonds. As used herein, the term refers to both short chains, which also commonly are referred to in the art as peptides, oligopeptides and oligomers, for example, and to longer chains, which generally are referred to in the art as proteins, of which there are many types.

In certain embodiments, a polypeptide is or may comprise a chimeric or “fusion protein.” As used herein, a “chimeric protein” or “fusion protein” comprises all or part (preferably a biologically active part) of a first protein operably linked to a heterologous second polypeptide (i.e., a polypeptide other than the first protein). Within the fusion protein, the term “operably linked” is intended to indicate that the first protein or segment thereof and the heterologous polypeptide are fused in-frame to each other. The heterologous polypeptide can be fused to the amino-terminus or the carboxyl-terminus of the first protein or segment.

A “polyamide” is a polymeric molecule with repeating units linked by amide binds. Proteins are examples of naturally occurring polyamides. In some embodiments, a polyamide comprises a peptide nucleic acid (PNA).

A “peptide nucleic acid” (“PNA”) is a molecule in which one or more amino acid units in the PNA have an amide containing backbone, e.g., aminoethyl-glycine, similar to a peptide backbone, with a nucleic acid side chain in place of the amino acid side chain. Peptide nucleic acids (PNA) are known to hybridize complementary DNA and RNA with higher affinity than their oligonucleotide counterparts. This character of PNA not only makes them a stable hybrid with the nucleic acid side chains, but at the same time, the neutral backbone and hydrophobic side chains result in a hydrophobic unit within the polypeptide. The nucleic acid side chain includes, but is not limited to, a purine or a pyrimidine side chain such as adenine, cytosine, guanine, thymine and uracil. In one embodiment, the nucleic acid side chain includes a nucleoside analog as described herein.

In one embodiment, a site-specific FOXP3 targeting moiety of the invention comprises a polyamide. Suitable polyamides for use in the agents and compositions of the invention are known in the art.

In one embodiment, a site-specific FOXP3 targeting moiety of the invention comprises a polynucleotide. In some embodiments, the nucleotide sequence of the polynucleotide encodes a FOXP3 gene or a FOXP3 expression product. In some embodiments, the nucleotide sequence of the polynucleotide does not include a FOXP3 coding sequence or a FOXP3 expression product. For example, in some embodiments, a site-specific FOXP3 targeting moiety of the invention comprises a polynucleotide that hybridizes to a target expression control region, e.g., a promoter, an anchor sequence, or a DNA region around or proximally upstream of the transcription starting site. In some embodiments, the nucleotide sequence of the polynucleotide is a complement of a target DNA region around or proximally upstream of the transcription starting site, or has a sequence that is at least 80%, at least 85%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical to a complement of the target sequence.

The polynucleotides of the invention may include deoxynucleotides, ribonucleotides, modified deoxynucleotides, modified ribonucleotides (e.g., chemical modifications, such as modifications that alter the backbone linkages, sugar molecules, and/or nucleic acid bases), and artificial nucleic acids. In some embodiments, the polynucleotide includes, but is not limited to, genomic DNA, cDNA, peptide nucleic acids (PNA) or peptide oligonucleotide conjugates, locked nucleic acids (LNA), bridged nucleic acids (BNA), polyamides, triplex forming oligonucleotides, modified DNA, antisense DNA oligonucleotides, tRNA, mPvNA, rPvNA, modified RNA, miRNA, gRNA, and siRNA or other RNA or DNA molecules.

In some embodiments, the polynucleotides of the invention have a length from about 2 to about 5000 nts, about 10 to about 100 nts, about 50 to about 150 nts, about 100 to about 200 nts, about 150 to about 250 nts, about 200 to about 300 nts, about 250 to about 350 nts, about 300 to about 500 nts, about 10 to about 1000 nts, about 50 to about 1000 nts, about 100 to about 1000 nts, about 1000 to about 2000 nts, about 2000 to about 3000 nts, about 3000 to about 4000 nts, about 4000 to about 5000 nts, or any range therebetween.

The polynucleotides of the invention may include nucleosides, e.g., purines or pyrimidines, e.g., adenine, cytosine, guanine, thymine and uracil. In some embodiments, the polynucleotides include one or more nucleoside analogs. The nucleoside analog includes, but is not limited to, a nucleoside analog, such as 5-fluorouracil; 5-bromouracil, 5-chlorouracil, 5-iodouracil, hypoxanthine, xanthine, 4-acetylcytosine, 4-methylbenzimidazole, 5-(carboxyhydroxylmethyl) uracil, 5-carboxymethylaminomethyl-2-thiouridine, 5-carboxymethylaminomethyluracil, dihydrouracil, dihydrouridine, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, 2,6-diaminopurine, 3-nitropyrrole, inosine, thiouridine, queuosine, wyosine, diaminopurine, isoguanine, isocytosine, diaminopyrimidine, 2,4-difluorotoluene, isoquinoline, pyrrolo[2,3-]pyridine, and any others that can base pair with a purine or a pyrimidine side chain.

In some embodiments, the site-specific FOXP3 targeting moieties of the invention comprising a polynucleotide encoding a polypeptide that comprises a DNA-binding domain (DBD), or fragment thereof, of a zinc finger polypeptide (ZNF) or a transcription activator-like effector (TALE) polypeptide, that is engineered to specifically target a FOXP3 expression control region to modulate expression of a FOXP3 gene.

The design and preparation of such zinc finger polypeptides which specifically bind to a DNA target region of interest, such as a FOXP3 expression control region, is well known in the art. For example, zinc finger (ZNF) proteins contain a DNA binding motif that specifically binds a triplet of nucleotides. Thus to design and prepare the site-specific FOXP3 targeting moieties of the invention, a modular assembly process which includes combining separate zinc finger DNA binding domains that can each recognize a specific 3-basepair DNA sequence to generate 3-finger, 4-, 5-, 6-, 6-, or 8-zinc finger polypeptide that recognizes specific target sites ranging from 9 basepairs to 24 basepairs in length may be used. Another suitable method may include 2-finger modules to generate ZNF polynucleotides with up to six individual zinc fingers. See, e.g., Shukla V K, et al., Nature. 459 (7245) 2009: 437-41; Dreier B, et al., JBC. 280 (42) 2005: 35588-97; Dreier B, et al, JBC 276 (31) 2001: 29466-78; Bae K H, et al., Nature Biotechnology. 21 (3) 2003: 275-80.

In some embodiments, a site-specific FOXP3 targeting moiety of the invention comprises a polynucleotide encoding a polypeptide that comprises a DNA-binding domain (DBD), or fragment thereof, of a zinc finger, that is engineered to specifically target a FOXP3 expression control region to modulate expression of a FOXP3 gene. Exemplary amino acid sequences encoding a zinc finger that binds to a nucleotide triplet suitable for use in the present invention are provide in Table 1A below. (See, e.g., Gersbach et al., Synthetic Zinc Finger Proteins: The Advent of Targeted Gene Regulation and Genome Modification Technologies).

TABLE 1A

| Amino Acid | | |
|---|---|---|
| Sequence of | | |
| Zing Finger | | |
| DNA Binding | | |
| Domain | Nucleotide | SEQ ID |
| (Finger) | Triplet | NO: |
| RKDALRG | TTG | 12 |
| TTGALTE | CTT | 13 |
| QRHHLVE | CTC | 14 |
| QNSTLTE | CTA | 15 |
| RNDALTE | CTG | 16 |
| HKNALQN | ATT | 17 |
| RRSACRR | ATC | 18 |
| QKSSLIA | ATA | 19 |
| RRDELNV | ATG | 20 |
| TSGSLVR | GTT | 21 |
| DPGALVR | GTC | 22 |
| QSSSLVR | GTA | 23 |
| RSDELVR | GTG | 24 |
| RLRDIQF | TCT | 25 |
| RSDERKR | TCC | 26 |
| RSDHLTT | TCA | 27 |
| RLRALDR | TCG | 28 |
| TKNSLTE | CCT | 29 |
| SKKHLAE | CCC | 30 |
| TSHSLTE | CCA | 31 |
| RNDTLTE | CCG | 32 |
| THLDLIR | ACT | 33 |
| DKKDLTR | ACC | 34 |
| SPADLTR | ACA | 35 |
| RTDTLRD | ACG | 36 |
| TSGELVR | GCT | 37 |
| DCRDLAR | GCC | 38 |
| QSGDLRR | GCA | 39 |
| RSDDLVR | GCG | 40 |
| ARGNLRT | TAT | 41 |
| SRGNLKS | TAC | 42 |
| QASNLIS | TAA | 43 |
| REDNLHT | TAG | 44 |
| TSGNLTE | CAT | 45 |
| SKKALTE | CAC | 46 |
| QSGNLTE | CAA | 47 |
| RADNLTE | CAG | 48 |
| TTGNLTV | AAT | 49 |
| DSGNLRV | AAC | 50 |
| QRANLRA | AAA | 51 |
| RKDNLKN | AAG | 52 |
| TSGNLVR | GAT | 53 |
| DPGNLVR | GAC | 54 |
| QSSNLVR | GAA | 55 |
| RSDNLVR | GAG | 56 |
| APKALGW | TGC | 57 |
| QAGHLAS | TGA | 58 |
| RSDHLTT | TGG | 27 |
| SRRTCRA | CGT | 59 |
| HTGHLLE | CGC | 60 |
| QSGHLTE | CGA | 66 |
| RSDKLTE | CGG | 67 |
| HRTTLTN | AGT | 68 |
| ERSHLRE | AGC | 69 |
| QLAHLRA | AGA | 70 |
| RSDHLTN | AGG | 71 |
| TSGHLVR | GGT | 72 |
| DPGHLVR | GGC | 73 |
| QRAHLER | GGA | 74 |
| RSDKLVR | GGG | 75 |

A zinc finger DNA binding domain comprises an N-terminal region and a C-terminal region with the “fingers” that bind to the target DNA sequence in between. The N-terminal region generally is 7 amino acids in length. The C-terminal region is generally 6 amino acids in length. Thus, the N-terminal region generally comprises the amino acid sequence of X₁X₂X₃X₄X₅X₆X₇. “X” can be any amino acid. In some embodiments, the N-terminal region comprises the exemplary amino acid sequence of LEPGEKP (SEQ ID NO: 76). “X” can be any amino acid. The C-terminal region generally comprises the amino acid sequence of X₂₅X₂₆X₂₇X₂₈X₂₉X₃₀. In certain embodiments, the C-terminal region comprises the exemplary amino acid sequence of TGKKTS (SEQ ID NO: 77).

Each finger in the DNA binding domain is flanked by a N-terminal backbone located to the N-terminus of the finger and a C-terminal backbone located to the C-terminus of the finger. The N-terminal backbone of the finger generally is 11 amino acids long with two conservative cysteines (C) locate at 3′ and 6^{th} positions. Thus, the N-terminal backbone of the finger generally comprises the amino acid sequence of X₈X₉CX₁₀X₁₁CX₁₂X₁₃X₁₄X₁₅X₁₆. “X” can be any amino acid. The C-terminal backbone of the finger generally is 5 amino acids long with two conservative histines (H) located at 1^{st} and 5^{th} positions. Thus, the C-terminal backbone of the finger generally comprises the amino acid sequence of HX₁₇X₁₈X₁₉H. “X” can be any amino acid. In some embodiments, the N-terminal backbone comprises the exemplary amino acid sequence of YKCPECGKSFS (SEQ ID No: 61) and the C-terminal backbone comprises the exemplary amino acid sequence of HQRTH (SEQ ID No: 62). Two “fingers” are linked through a linker. A linker generally is 5 amino acids in length and comprises the amino acid sequence of X₂₀X₂₁X₂₂X₂₃X₂₄ “X” can be any amino acid. In certain embodiments, the linker comprises the exemplary amino acid sequence of TGEKP (SEQ ID No: 63). Thus, the zinc finger of a site specific FOXP3 site-specific disrupting agent has a structure as follows: (N-terminal backbone—finger—C-terminal backbone—linker)ₙ and the zinc finger DNA binding domain of a site specific FOXP3 site-specific disrupting agent has a structure as follows: [N-terminal region (N-terminal backbone—finger—C-terminal backbone—linker), C-terminal region]. “N” represents the number of triplets of nucleotides to which the zinc finger DNA binding domain and, thus, to which the FOXP3 site-specific disrupting agent binds.

The “finger” amino acid sequences of four nucleotide triplets are unknown, however, if such a triplet is identified in a target area of interest, two “linker span sequences”—linker span 1 and linker span 2—are useful to circumvent the issue. Linker span 1 is used to skip one base pair if a “finger” amino acid sequence of a triplet is not available. Linker span 2 is used to skip 2 base pairs if a “finger” amino acid sequence of a triplet is not available. Linker span 1 is generally 12 amino acids long. Linker span 2 is generally 16 amino acids long. Thus, linker span 1 generally comprises the amino acid sequence of X₃₁X₃₂X₃₃X₃₄X₃₅X₃₆X₃₇X₃₈X₃₉X₄₀X₄₁X₄₂ Linker span 2 generally comprises the amino acid sequence of X₄₃X₄₄X₄₅X₄₆X₄₇X₄₈X₄₉X₅₀X₅₁X₅₂X₅₃X₅₄X₅₅X₅₆X₅₇X₅₈. In some embodiments, linker span 1 comprises the amino acid sequence of THPRAPIPKPFQ (SEQ ID NO: 78). In certain embodiments, linker span 2 comprises the amino acid sequence of TPNPHRRTDPSHKPFQ (SEQ ID NO: 79). When linker span 1 and/or linker span 2 is used, the finger—linker span 1/span 2—finger comprises the structure as follows: N-terminal back bone—finger—C-terminal backbone—linker span 1/span 2— N-terminal backbone—finger—C-terminal backbone—linker.

Table 1B provides the amino acid sequence of exemplary zinc finger DNA binding domains for use in the present invention and their corresponding target regions.

In some embodiments, a zinc finger DNA binding domain suitable for use in the disrupting agents of the invention comprises an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% amino acid identity to the entire amino acid sequence of any one of the zinc finger DNA binding domains provided in Table 1B.

TABLE 1B

| | | SEQ | | SEQ |
|---|---|---|---|---|
| | Long | ID | | ID |
| Strand | Target | NO: | ZF amino acid sequence | NO: |
| + | cacTcaccttg | 80 | LEPGEKPYKCPECGKSFSDPGNLVRHQRTHTGEKPYKCPECGKSFSRSDELVRHQRTHTGEKPYKCPECG | 609 |
| | gtgaagtggac | | KSFSQSSNLVRHQRTHTGEKPYKCPECGKSFSTSGHLVRHQRTHTGEKPYKCPECGKSFSTTGALTEHQR | |
| | | | THTGEKPYKCPECGKSFSSKKALTEHQRTHTHPRAPIPKPFQYKCPECGKSFSSKKALTEHQRTHTGKKTS | |
| + | accTtggtgaa | 81 | LEPGEKPYKCPECGKSFSSPADLTRHQRTHTGEKPYKCPECGKSFSRNDALTEHQRTHTGEKPYKCPECG | 610 |
| | gtggactgaca | | KSFSQRAHLERHQRTHTGEKPYKCPECGKSFSHRTTLTNHQRTHTGEKPYKCPECGKSFSQAGHLASHQR | |
| | | | THTGEKPYKCPECGKSFSRSDHLTTHQRTHTHPRAPIPKPFQYKCPECGKSFSDKKDLTRHQRTHTGKKTS | |
| + | ccttggtgaagt | 82 | LEPGEKPYKCPECGKSFSSPADLTRHQRTHTGEKPYKCPECGKSFSRNDALTEHQRTHTGEKPYKCPECG | 611 |
| | ggactgaca | | KSFSQRAHLERHQRTHTGEKPYKCPECGKSFSHRTTLTNHQRTHTGEKPYKCPECGKSFSQAGHLASHQR | |
| | | | THTGEKPYKCPECGKSFSRSDHLTTHQRTHTGEKPYKCPECGKSFSTKNSLTEHQRTHTGKKTS | |
| + | tggtgaagtgga | 83 | LEPGEKPYKCPECGKSFSQSSNLVRHQRTHTGEKPYKCPECGKSFSSPADLTRHQRTHTGEKPYKCPECG | 612 |
| | ctgacagaa | | KSFSRNDALTEHQRTHTGEKPYKCPECGKSFSQRAHLERHQRTHTGEKPYKCPECGKSFSHRTTLTNHQR | |
| | | | THTGEKPYKCPECGKSFSQAGHLASHQRTHTGEKPYKCPECGKSFSRSDHLTTHQRTHTGKKTS | |
| + | tgaagtggactg | 84 | LEPGEKPYKCPECGKSFSRKDNLKNHQRTHTGEKPYKCPECGKSFSQSSNLVRHQRTHTGEKPYKCPECG | 613 |
| | acagaaaag | | KSFSSPADLTRHQRTHTGEKPYKCPECGKSFSRNDALTEHQRTHTGEKPYKCPECGKSFSQRAHLERHQR | |
| | | | THTGEKPYKCPECGKSFSHRTTLTNHQRTHTGEKPYKCPECGKSFSQAGHLASHQRTHTGKKTS | |
| + | agtggactgaca | 85 | LEPGEKPYKCPECGKSFSTSGNLVRHQRTHTGEKPYKCPECGKSFSRKDNLKNHQRTHTGEKPYKCPECG | 614 |
| | gaaaaggat | | KSFSQSSNLVRHQRTHTGEKPYKCPECGKSFSSPADLTRHQRTHTGEKPYKCPECGKSFSRNDALTEHQR | |
| | | | THTGEKPYKCPECGKSFSQRAHLERHQRTHTGEKPYKCPECGKSFSHRTTLTNHQRTHTGKKTS | |
| + | ggactgacaga | 86 | LEPGEKPYKCPECGKSFSRADNLTEHQRTHTGEKPYKCPECGKSFSTSGNLVRHQRTHTGEKPYKCPECG | 615 |
| | aaaggatcag | | KSFSRKDNLKNHQRTHTGEKPYKCPECGKSFSQSSNLVRHQRTHTGEKPYKCPECGKSFSSPADLTRHQR | |
| | | | THTGEKPYKCPECGKSFSRNDALTEHQRTHTGEKPYKCPECGKSFSQRAHLERHQRTHTGKKTS | |
| + | ctgacagaaaa | 87 | LEPGEKPYKCPECGKSFSTKNSLTEHQRTHTGEKPYKCPECGKSFSRADNLTEHQRTHTGEKPYKCPECG | 616 |
| | ggatcagcct | | KSFSTSGNLVRHQRTHTGEKPYKCPECGKSFSRKDNLKNHQRTHTGEKPYKCPECGKSFSQSSNLVRHQR | |
| | | | THTGEKPYKCPECGKSFSSPADLTRHQRTHTGEKPYKCPECGKSFSRNDALTEHQRTHTGKKTS | |
| + | acagaaaagga | 88 | LEPGEKPYKCPECGKSFSDPGHLVRHQRTHTGEKPYKCPECGKSFSTKNSLTEHQRTHTGEKPYKCPECG | 617 |
| | tcagcctggc | | KSFSRADNLTEHQRTHTGEKPYKCPECGKSFSTSGNLVRHQRTHTGEKPYKCPECGKSFSRKDNLKNHQR | |
| | | | THTGEKPYKCPECGKSFSQSSNLVRHQRTHTGEKPYKCPECGKSFSSPADLTRHQRTHTGKKTS | |
| + | gaaaaggatca | 89 | LEPGEKPYKCPECGKSFSRSDELVRHQRTHTPNPHRRTDPSHKPFQYKCPECGKSFSDPGHLVRHQRTHT | 618 |
| | gcctggcTTgt | | GEKPYKCPECGKSFSTKNSLTEHQRTHTGEKPYKCPECGKSFSRADNLTEHQRTHTGEKPYKCPECGKSF | |
| | g | | STSGNLVRHQRTHTGEKPYKCPECGKSFSRKDNLKNHQRTHTGEKPYKCPECGKSFSQSSNLVRHQRTHT | |
| | | | GKKTS | |
| + | aaggatcagcct | 90 | LEPGEKPYKCPECGKSFSQRAHLERHQRTHTGEKPYKCPECGKSFSRSDELVRHQRTHTPNPHRRTDPSH | 619 |
| | ggcTTgtggg | | KPFQYKCPECGKSFSDPGHLVRHQRTHTGEKPYKCPECGKSFSTKNSLTEHQRTHTGEKPYKCPECGKSF | |
| | a | | SRADNLTEHQRTHTGEKPYKCPECGKSFSTSGNLVRHQRTHTGEKPYKCPECGKSFSRKDNLKNHQRTH | |
| | | | TGKKTS | |
| + | gatcagcctggc | 91 | LEPGEKPYKCPECGKSFSDSGNLRVHQRTHTGEKPYKCPECGKSFSQRAHLERHQRTHTGEKPYKCPECG | 620 |
| | TTgtgggaaa | | KSFSRSDELVRHQRTHTPNPHRRTDPSHKPFQYKCPECGKSFSDPGHLVRHQRTHTGEKPYKCPECGKSFS | |
| | c | | TKNSLTEHQRTHTGEKPYKCPECGKSFSRADNLTEHQRTHTGEKPYKCPECGKSFSTSGNLVRHQRTHTG | |
| | | | KKTS | |
| + | cagcctggcTT | 92 | LEPGEKPYKCPECGKSFSDPGALVRHQRTHTHPRAPIPKPFQYKCPECGKSFSDSGNLRVHQRTHTGEKPY | 621 |
| | gtgggaaacTg | | KCPECGKSFSQRAHLERHQRTHTGEKPYKCPECGKSFSRSDELVRHQRTHTPNPHRRTDPSHKPFQYKCP | |
| | tc | | ECGKSFSDPGHLVRHQRTHTGEKPYKCPECGKSFSTKNSLTEHQRTHTGEKPYKCPECGKSFSRADNLTE | |
| | | | HQRTHTGKKTS | |
| + | cctggcTTgtg | 93 | LEPGEKPYKCPECGKSFSRTDTLRDHQRTHTGEKPYKCPECGKSFSDPGALVRHQRTHTHPRAPIPKPFQY | 622 |
| | ggaaacTgtca | | KCPECGKSFSDSGNLRVHQRTHTGEKPYKCPECGKSFSQRAHLERHQRTHTGEKPYKCPECGKSFSRSDE | |
| | cg | | LVRHQRTHTPNPHRRTDPSHKPFQYKCPECGKSFSDPGHLVRHQRTHTGEKPYKCPECGKSFSTKNSLTE | |
| | | | HQRTHTGKKTS | |
| + | gtgaagtggact | 94 | LEPGEKPYKCPECGKSFSQRANLRAHQRTHTGEKPYKCPECGKSFSQLAHLRAHQRTHTGEKPYKCPECG | 623 |
| | gacagaaaa | | KSFSDPGNLVRHQRTHTGEKPYKCPECGKSFSTHLDLIRHQRTHTGEKPYKCPECGKSFSRSDHLTTHQRT | |
| | | | HTGEKPYKCPECGKSFSRKDNLKNHQRTHTGEKPYKCPECGKSFSRSDELVRHQRTHTGKKTS | |
| + | aagtggactgac | 142 | LEPGEKPYKCPECGKSFSQRAHLERHQRTHTGEKPYKCPECGKSFSQRANLRAHQRTHTGEKPYKCPECG | 624 |
| | agaaaagga | | KSFSQLAHLRAHQRTHTGEKPYKCPECGKSFSDPGNLVRHQRTHTGEKPYKCPECGKSFSTHLDLIRHQR | |
| | | | THTGEKPYKCPECGKSFSRSDHLTTHQRTHTGEKPYKCPECGKSFSRKDNLKNHQRTHTGKKTS | |
| + | tggactgacaga | 143 | LEPGEKPYKCPECGKSFSRADNLTEHQRTHTHPRAPIPKPFQYKCPECGKSFSQRAHLERHQRTHTGEKPY | 625 |
| | aaaggaTcag | | KCPECGKSFSQRANLRAHQRTHTGEKPYKCPECGKSFSQLAHLRAHQRTHTGEKPYKCPECGKSFSDPGN | |
| | | | LVRHQRTHTGEKPYKCPECGKSFSTHLDLIRHQRTHTGEKPYKCPECGKSFSRSDHLTTHQRTHTGKKTS | |
| + | actgacagaaa | 144 | LEPGEKPYKCPECGKSFSTKNSLTEHQRTHTGEKPYKCPECGKSFSRADNLTEHQRTHTHPRAPIPKPFQY | 626 |
| | aggaTcagcct | | KCPECGKSFSQRAHLERHQRTHTGEKPYKCPECGKSFSQRANLRAHQRTHTGEKPYKCPECGKSFSQLAH | |
| | | | LRAHQRTHTGEKPYKCPECGKSFSDPGNLVRHQRTHTGEKPYKCPECGKSFSTHLDLIRHQRTHTGKKTS | |
| + | gacagaaaagg | 145 | LEPGEKPYKCPECGKSFSDPGHLVRHQRTHTGEKPYKCPECGKSFSTKNSLTEHQRTHTGEKPYKCPECG | 627 |
| | aTcagcctggc | | KSFSRADNLTEHQRTHTHPRAPIPKPFQYKCPECGKSFSQRAHLERHQRTHTGEKPYKCPECGKSFSQRAN | |
| | | | LRAHQRTHTGEKPYKCPECGKSFSQLAHLRAHQRTHTGEKPYKCPECGKSFSDPGNLVRHQRTHTGKKT | |
| | | | S | |
| + | agaaaaggaTc | 146 | LEPGEKPYKCPECGKSFSRSDELVRHQRTHTPNPHRRTDPSHKPFQYKCPECGKSFSDPGHLVRHQRTHT | 628 |
| | agcctggcTT | | GEKPYKCPECGKSFSTKNSLTEHQRTHTGEKPYKCPECGKSFSRADNLTEHQRTHTHPRAPIPKPFQYKCP | |
| | gtg | | ECGKSFSQRAHLERHQRTHTGEKPYKCPECGKSFSQRANLRAHQRTHTGEKPYKCPECGKSFSQLAHLR | |
| | | | AHQRTHTGKKTS | |
| + | aaaggaTcagc | 147 | LEPGEKPYKCPECGKSFSQRAHLERHQRTHTGEKPYKCPECGKSFSRSDELVRHQRTHTPNPHRRTDPSH | 629 |
| | ctggcTTgtgg | | KPFQYKCPECGKSFSDPGHLVRHQRTHTGEKPYKCPECGKSFSTKNSLTEHQRTHTGEKPYKCPECGKSF | |
| | ga | | SRADNLTEHQRTHTHPRAPIPKPFQYKCPECGKSFSQRAHLERHQRTHTGEKPYKCPECGKSFSQRANLR | |
| | | | AHQRTHTGKKTS | |
| + | ggaTcagcctg | 148 | LEPGEKPYKCPECGKSFSDSGNLRVHQRTHTGEKPYKCPECGKSFSQRAHLERHQRTHTGEKPYKCPECG | 630 |
| | gcTTgtggga | | KSFSRSDELVRHQRTHTPNPHRRTDPSHKPFQYKCPECGKSFSDPGHLVRHQRTHTGEKPYKCPECGKSFS | |
| | aac | | TKNSLTEHQRTHTGEKPYKCPECGKSFSRADNLTEHQRTHTHPRAPIPKPFQYKCPECGKSFSQRAHLERH | |
| | | | QRTHTGKKTS | |
| + | agcctggctTgt | 149 | LEPGEKPYKCPECGKSFSDPGALVRHQRTHTHPRAPIPKPFQYKCPECGKSFSDSGNLRVHQRTHTGEKPY | 631 |
| | gggaaacTgtc | | KCPECGKSFSQRAHLERHQRTHTGEKPYKCPECGKSFSRSDELVRHQRTHTHPRAPIPKPFQYKCPECGKS | |
| | | | FSTSGELVRHQRTHTGEKPYKCPECGKSFSRNDALTEHQRTHTGEKPYKCPECGKSFSERSHLREHQRTH | |
| | | | TGKKTS | |
| + | ctggctTgtgg | 150 | LEPGEKPYKCPECGKSFSRTDTLRDHQRTHTGEKPYKCPECGKSFSDPGALVRHQRTHTHPRAPIPKPFQY | 632 |
| | gaaacTgtcac | | KCPECGKSFSDSGNLRVHQRTHTGEKPYKCPECGKSFSQRAHLERHQRTHTGEKPYKCPECGKSFSRSDE | |
| | g | | LVRHQRTHTHPRAPIPKPFQYKCPECGKSFSTSGELVRHQRTHTGEKPYKCPECGKSFSRNDALTEHQRTH | |
| | | | TGKKTS | |
| + | gcctggcttgtg | 151 | LEPGEKPYKCPECGKSFSDPGALVRHQRTHTHPRAPIPKPFQYKCPECGKSFSDSGNLRVHQRTHTGEKPY | 633 |
| | ggaaacTgtc | | KCPECGKSFSQRAHLERHQRTHTGEKPYKCPECGKSFSRSDELVRHQRTHTGEKPYKCPECGKSFSTTGA | |
| | | | LTEHQRTHTGEKPYKCPECGKSFSRSDHLTTHQRTHTGEKPYKCPECGKSFSDCRDLARHQRTHTGKKTS | |
| + | tggcttgtggga | 152 | LEPGEKPYKCPECGKSFSRTDTLRDHQRTHTGEKPYKCPECGKSFSDPGALVRHQRTHTHPRAPIPKPFQY | 634 |
| | aacTgtcacg | | KCPECGKSFSDSGNLRVHQRTHTGEKPYKCPECGKSFSQRAHLERHQRTHTGEKPYKCPECGKSFSRSDE | |
| | | | LVRHQRTHTGEKPYKCPECGKSFSTTGALTEHQRTHTGEKPYKCPECGKSFSRSDHLTTHQRTHTGKKTS | |
| + | gggaaactgTc | 153 | LEPGEKPYKCPECGKSFSQRANLRAHQRTHTGEKPYKCPECGKSFSQSGNLTEHQRTHTHPRAPIPKPFQY | 635 |
| | acgtaTcaaaa | | KCPECGKSFSQSSSLVRHQRTHTGEKPYKCPECGKSFSSKKALTEHQRTHTHPRAPIPKPFQYKCPECGKS | |
| | a | | FSRNDALTEHQRTHTGEKPYKCPECGKSFSQRANLRAHQRTHTGEKPYKCPECGKSFSRSDKLVRHQRT | |
| | | | HTGKKTS | |
| + | aaactgTcacg | 154 | LEPGEKPYKCPECGKSFSQSGNLTEHQRTHTGEKPYKCPECGKSFSQRANLRAHQRTHTGEKPYKCPECG | 636 |
| | taTcaaaaaca | | KSFSQSGNLTEHQRTHTHPRAPIPKPFQYKCPECGKSFSQSSSLVRHQRTHTGEKPYKCPECGKSFSSKKA | |
| | a | | LTEHQRTHTHPRAPIPKPFQYKCPECGKSFSRNDALTEHQRTHTGEKPYKCPECGKSFSQRANLRAHQRT | |
| | | | HTGKKTS | |
| + | ctgTcacgtaT | 155 | LEPGEKPYKCPECGKSFSTTGALTEHQRTHTGEKPYKCPECGKSFSQSGNLTEHQRTHTGEKPYKCPECG | 637 |
| | caaaaacaactt | | KSFSQRANLRAHQRTHTGEKPYKCPECGKSFSQSGNLTEHQRTHTHPRAPIPKPFQYKCPECGKSFSQSSS | |
| | | | LVRHQRTHTGEKPYKCPECGKSFSSKKALTEHQRTHTHPRAPIPKPFQYKCPECGKSFSRNDALTEHQRT | |
| | | | HTGKKTS | |
| + | cacgtaTcaaa | 156 | LEPGEKPYKCPECGKSFSTSGELVRHQRTHTHPRAPIPKPFQYKCPECGKSFSTTGALTEHQRTHTGEKPY | 638 |
| | aacaacttTgct | | KCPECGKSFSQSGNLTEHQRTHTGEKPYKCPECGKSFSQRANLRAHQRTHTGEKPYKCPECGKSFSQSGN | |
| | | | LTEHQRTHTHPRAPIPKPFQYKCPECGKSFSQSSSLVRHQRTHTGEKPYKCPECGKSFSSKKALTEHQRTH | |
| | | | TGKKTS | |
| + | cttTTataccga | 157 | LEPGEKPYKCPECGKSFSDKKDLTRHQRTHTGEKPYKCPECGKSFSQRANLRAHQRTHTGEKPYKCPECG | 639 |
| | gaagaaaaacc | | KSFSQLAHLRAHQRTHTGEKPYKCPECGKSFSQLAHLRAHQRTHTGEKPYKCPECGKSFSRNDTLTEHQR | |
| | | | THTGEKPYKCPECGKSFSQKSSLIAHQRTHTPNPHRRTDPSHKPFQYKCPECGKSFSTTGALTEHQRTHTG | |
| | | | KKTS | |
| + | ataccgagaag | 158 | LEPGEKPYKCPECGKSFSRTDTLRDHQRTHTGEKPYKCPECGKSFSDKKDLTRHQRTHTGEKPYKCPECG | 640 |
| | aaaaaccacg | | KSFSQRANLRAHQRTHTGEKPYKCPECGKSFSQLAHLRAHQRTHTGEKPYKCPECGKSFSQLAHLRAHQ | |
| | | | RTHTGEKPYKCPECGKSFSRNDTLTEHQRTHTGEKPYKCPECGKSFSQKSSLIAHQRTHTGKKTS | |
| + | ccgagaagaaa | 159 | LEPGEKPYKCPECGKSFSRNDALTEHQRTHTGEKPYKCPECGKSFSRTDTLRDHQRTHTGEKPYKCPECG | 641 |
| | aaccacgctg | | KSFSDKKDLTRHQRTHTGEKPYKCPECGKSFSQRANLRAHQRTHTGEKPYKCPECGKSFSQLAHLRAHQ | |
| | | | RTHTGEKPYKCPECGKSFSQLAHLRAHQRTHTGEKPYKCPECGKSFSRNDTLTEHQRTHTGKKTS | |
| + | agaagaaaaac | 160 | LEPGEKPYKCPECGKSFSRTDTLRDHQRTHTHPRAPIPKPFQYKCPECGKSFSRNDALTEHQRTHTGEKPY | 642 |
| | cacgctgTacg | | KCPECGKSFSRTDTLRDHQRTHTGEKPYKCPECGKSFSDKKDLTRHQRTHTGEKPYKCPECGKSFSQRAN | |
| | | | LRAHQRTHTGEKPYKCPECGKSFSQLAHLRAHQRTHTGEKPYKCPECGKSFSQLAHLRAHQRTHTGKKT | |
| | | | S | |
| + | agaaaaaccac | 161 | LEPGEKPYKCPECGKSFSRSDELVRHQRTHTGEKPYKCPECGKSFSRTDTLRDHQRTHTHPRAPIPKPFQY | 643 |
| | gctgTacggtg | | KCPECGKSFSRNDALTEHQRTHTGEKPYKCPECGKSFSRTDTLRDHQRTHTGEKPYKCPECGKSFSDKKD | |
| | | | LTRHQRTHTGEKPYKCPECGKSFSQRANLRAHQRTHTGEKPYKCPECGKSFSQLAHLRAHQRTHTGKKT | |
| | | | S | |
| + | aaaaccacgct | 162 | LEPGEKPYKCPECGKSFSRSDHLTTHQRTHTGEKPYKCPECGKSFSRSDELVRHQRTHTGEKPYKCPECG | 644 |
| | gTacggtgtgg | | KSFSRTDTLRDHQRTHTHPRAPIPKPFQYKCPECGKSFSRNDALTEHQRTHTGEKPYKCPECGKSFSRTDT | |
| | | | LRDHQRTHTGEKPYKCPECGKSFSDKKDLTRHQRTHTGEKPYKCPECGKSFSQRANLRAHQRTHTGKKT | |
| | | | S | |
| + | accacgctgTa | 163 | LEPGEKPYKCPECGKSFSRKDNLKNHQRTHTGEKPYKCPECGKSFSRSDHLTTHQRTHTGEKPYKCPECG | 645 |
| | cggtgtggaag | | KSFSRSDELVRHQRTHTGEKPYKCPECGKSFSRTDTLRDHQRTHTHPRAPIPKPFQYKCPECGKSFSRNDA | |
| | | | LTEHQRTHTGEKPYKCPECGKSFSRTDTLRDHQRTHTGEKPYKCPECGKSFSDKKDLTRHQRTHTGKKTS | |
| + | acgctgTacgg | 164 | LEPGEKPYKCPECGKSFSRNDTLTEHQRTHTGEKPYKCPECGKSFSRKDNLKNHQRTHTGEKPYKCPECG | 646 |
| | tgtggaagccg | | KSFSRSDHLTTHQRTHTGEKPYKCPECGKSFSRSDELVRHQRTHTGEKPYKCPECGKSFSRTDTLRDHQR | |
| | | | THTHPRAPIPKPFQYKCPECGKSFSRNDALTEHQRTHTGEKPYKCPECGKSFSRTDTLRDHQRTHTGKKTS | |
| + | ctgTacggtgt | 165 | LEPGEKPYKCPECGKSFSRADNLTEHQRTHTGEKPYKCPECGKSFSRNDTLTEHQRTHTGEKPYKCPECG | 647 |
| | ggaagccgcag | | KSFSRKDNLKNHQRTHTGEKPYKCPECGKSFSRSDHLTTHQRTHTGEKPYKCPECGKSFSRSDELVRHQR | |
| | | | THTGEKPYKCPECGKSFSRTDTLRDHQRTHTHPRAPIPKPFQYKCPECGKSFSRNDALTEHQRTHTGKKTS | |
| + | accgagaagaa | 166 | LEPGEKPYKCPECGKSFSTSGELVRHQRTHTGEKPYKCPECGKSFSSKKALTEHQRTHTGEKPYKCPECG | 648 |
| | aaaccacgct | | KSFSDSGNLRVHQRTHTGEKPYKCPECGKSFSQRANLRAHQRTHTGEKPYKCPECGKSFSRKDNLKNHQ | |
| | | | RTHTGEKPYKCPECGKSFSRSDNLVRHQRTHTGEKPYKCPECGKSFSDKKDLTRHQRTHTGKKTS | |
| + | gagaagaaaaa | 167 | LEPGEKPYKCPECGKSFSQSSSLVRHQRTHTGEKPYKCPECGKSFSTSGELVRHQRTHTGEKPYKCPECGK | 649 |
| | ccacgctgta | | SFSSKKALTEHQRTHTGEKPYKCPECGKSFSDSGNLRVHQRTHTGEKPYKCPECGKSFSQRANLRAHQRT | |
| | | | HTGEKPYKCPECGKSFSRKDNLKNHQRTHTGEKPYKCPECGKSFSRSDNLVRHQRTHTGKKTS | |
| + | aagaaaaacca | 168 | LEPGEKPYKCPECGKSFSRSDKLTEHQRTHTGEKPYKCPECGKSFSQSSSLVRHQRTHTGEKPYKCPECGK | 650 |
| | cgctgtacgg | | SFSTSGELVRHQRTHTGEKPYKCPECGKSFSSKKALTEHQRTHTGEKPYKCPECGKSFSDSGNLRVHQRT | |
| | | | HTGEKPYKCPECGKSFSQRANLRAHQRTHTGEKPYKCPECGKSFSRKDNLKNHQRTHTGKKTS | |
| + | aaaaaccacgct | 169 | LEPGEKPYKCPECGKSFSRSDELVRHQRTHTHPRAPIPKPFQYKCPECGKSFSRSDKLTEHQRTHTGEKPY | 651 |
| | gtacggTgtg | | KCPECGKSFSQSSSLVRHQRTHTGEKPYKCPECGKSFSTSGELVRHQRTHTGEKPYKCPECGKSFSSKKAL | |
| | | | TEHQRTHTGEKPYKCPECGKSFSDSGNLRVHQRTHTGEKPYKCPECGKSFSQRANLRAHQRTHTGKKTS | |
| + | aaccacgctgta | 170 | LEPGEKPYKCPECGKSFSQSSNLVRHQRTHTGEKPYKCPECGKSFSRSDELVRHQRTHTHPRAPIPKPFQY | 652 |
| | cggTgtggaa | | KCPECGKSFSRSDKLTEHQRTHTGEKPYKCPECGKSFSQSSSLVRHQRTHTGEKPYKCPECGKSFSTSGEL | |
| | | | VRHQRTHTGEKPYKCPECGKSFSSKKALTEHQRTHTGEKPYKCPECGKSFSDSGNLRVHQRTHTGKKTS | |
| + | cacgctgtacgg | 171 | LEPGEKPYKCPECGKSFSDCRDLARHQRTHTGEKPYKCPECGKSFSQSSNLVRHQRTHTGEKPYKCPECG | 653 |
| | Tgtggaagcc | | KSFSRSDELVRHQRTHTHPRAPIPKPFQYKCPECGKSFSRSDKLTEHQRTHTGEKPYKCPECGKSFSQSSSL | |
| | | | VRHQRTHTGEKPYKCPECGKSFSTSGELVRHQRTHTGEKPYKCPECGKSFSSKKALTEHQRTHTGKKTS | |
| + | gctgtacggTg | 172 | LEPGEKPYKCPECGKSFSQSGDLRRHQRTHTGEKPYKCPECGKSFSDCRDLARHQRTHTGEKPYKCPECG | 654 |
| | tggaagccgca | | KSFSQSSNLVRHQRTHTGEKPYKCPECGKSFSRSDELVRHQRTHTHPRAPIPKPFQYKCPECGKSFSRSDK | |
| | | | LTEHQRTHTGEKPYKCPECGKSFSQSSSLVRHQRTHTGEKPYKCPECGKSFSTSGELVRHQRTHTGKKTS | |
| + | gtacggTgtgg | 173 | LEPGEKPYKCPECGKSFSDPGNLVRHQRTHTGEKPYKCPECGKSFSQSGDLRRHQRTHTGEKPYKCPECG | 655 |
| | aagccgcagac | | KSFSDCRDLARHQRTHTGEKPYKCPECGKSFSQSSNLVRHQRTHTGEKPYKCPECGKSFSRSDELVRHQR | |
| | | | THTHPRAPIPKPFQYKCPECGKSFSRSDKLTEHQRTHTGEKPYKCPECGKSFSQSSSLVRHQRTHTGKKTS | |
| + | cgagaagaaaa | 174 | LEPGEKPYKCPECGKSFSQSSSLVRHQRTHTHPRAPIPKPFQYKCPECGKSFSHTGHLLEHQRTHTGEKPY | 656 |
| | accacgcTgta | | KCPECGKSFSTSHSLTEHQRTHTGEKPYKCPECGKSFSQRANLRAHQRTHTGEKPYKCPECGKSFSQSSNL | |
| | | | VRHQRTHTGEKPYKCPECGKSFSQSSNLVRHQRTHTGEKPYKCPECGKSFSQSGHLTEHQRTHTGKKTS | |
| + | gaagaaaaacc | 175 | LEPGEKPYKCPECGKSFSRSDKLTEHQRTHTGEKPYKCPECGKSFSQSSSLVRHQRTHTHPRAPIPKPFQY | 657 |
| | acgcTgtacgg | | KCPECGKSFSHTGHLLEHQRTHTGEKPYKCPECGKSFSTSHSLTEHQRTHTGEKPYKCPECGKSFSQRANL | |
| | | | RAHQRTHTGEKPYKCPECGKSFSQSSNLVRHQRTHTGEKPYKCPECGKSFSQSSNLVRHQRTHTGKKTS | |
| + | gaaaaaccacg | 176 | LEPGEKPYKCPECGKSFSRSDELVRHQRTHTHPRAPIPKPFQYKCPECGKSFSRSDKLTEHQRTHTGEKPY | 658 |
| | cTgtacggTgt | | KCPECGKSFSQSSSLVRHQRTHTHPRAPIPKPFQYKCPECGKSFSHTGHLLEHQRTHTGEKPYKCPECGKS | |
| | g | | FSTSHSLTEHQRTHTGEKPYKCPECGKSFSQRANLRAHQRTHTGEKPYKCPECGKSFSQSSNLVRHQRTH | |
| | | | TGKKTS | |
| + | aaaccacgcTg | 177 | LEPGEKPYKCPECGKSFSQSSNLVRHQRTHTGEKPYKCPECGKSFSRSDELVRHQRTHTHPRAPIPKPFQY | 659 |
| | tacggTgtgga | | KCPECGKSFSRSDKLTEHQRTHTGEKPYKCPECGKSFSQSSSLVRHQRTHTHPRAPIPKPFQYKCPECGKS | |
| | a | | FSHTGHLLEHQRTHTGEKPYKCPECGKSFSTSHSLTEHQRTHTGEKPYKCPECGKSFSQRANLRAHQRTH | |
| | | | TGKKTS | |
| + | ccacgcTgtac | 178 | LEPGEKPYKCPECGKSFSDCRDLARHQRTHTGEKPYKCPECGKSFSQSSNLVRHQRTHTGEKPYKCPECG | 660 |
| | ggTgtggaag | | KSFSRSDELVRHQRTHTHPRAPIPKPFQYKCPECGKSFSRSDKLTEHQRTHTGEKPYKCPECGKSFSQSSSL | |
| | cc | | VRHQRTHTHPRAPIPKPFQYKCPECGKSFSHTGHLLEHQRTHTGEKPYKCPECGKSFSTSHSLTEHQRTHT | |
| | | | GKKTS | |
| + | cgcTgtacgg | 179 | LEPGEKPYKCPECGKSFSQSGDLRRHQRTHTGEKPYKCPECGKSFSDCRDLARHQRTHTGEKPYKCPECG | 661 |
| | Tgtggaagccg | | KSFSQSSNLVRHQRTHTGEKPYKCPECGKSFSRSDELVRHQRTHTHPRAPIPKPFQYKCPECGKSFSRSDK | |
| | ca | | LTEHQRTHTGEKPYKCPECGKSFSQSSSLVRHQRTHTHPRAPIPKPFQYKCPECGKSFSHTGHLLEHQRTH | |
| | | | TGKKTS | |
| + | acggtgtggaa | 180 | LEPGEKPYKCPECGKSFSDKKDLTRHQRTHTGEKPYKCPECGKSFSRADNLTEHQRTHTGEKPYKCPECG | 662 |
| | gccgcagacc | | KSFSRNDTLTEHQRTHTGEKPYKCPECGKSFSRKDNLKNHQRTHTGEKPYKCPECGKSFSRSDHLTTHQR | |
| | | | THTGEKPYKCPECGKSFSRSDELVRHQRTHTGEKPYKCPECGKSFSRTDTLRDHQRTHTGKKTS | |
| + | cgatgagtgTg | 181 | LEPGEKPYKCPECGKSFSTTGNLTVHQRTHTHPRAPIPKPFQYKCPECGKSFSQAGHLASHQRTHTGEKPY | 663 |
| | tgcgctgaTaat | | KCPECGKSFSHTGHLLEHQRTHTGEKPYKCPECGKSFSRSDELVRHQRTHTHPRAPIPKPFQYKCPECGKS | |
| | | | FSRSDELVRHQRTHTGEKPYKCPECGKSFSQAGHLASHQRTHTGEKPYKCPECGKSFSQSGHLTEHQRTH | |
| | | | TGKKTS | |
| + | tgagtgTgtgc | 182 | LEPGEKPYKCPECGKSFSSKKALTEHQRTHTGEKPYKCPECGKSFSTTGNLTVHQRTHTHPRAPIPKPFQY | 664 |
| | gctgaTaatca | | KCPECGKSFSQAGHLASHQRTHTGEKPYKCPECGKSFSHTGHLLEHQRTHTGEKPYKCPECGKSFSRSDE | |
| | c | | LVRHQRTHTHPRAPIPKPFQYKCPECGKSFSRSDELVRHQRTHTGEKPYKCPECGKSFSQAGHLASHQRT | |
| | | | HTGKKTS | |
| + | gtgTgtgcgct | 183 | LEPGEKPYKCPECGKSFSRSDKLVRHQRTHTGEKPYKCPECGKSFSSKKALTEHQRTHTGEKPYKCPECG | 665 |
| | gaTaatcacgg | | KSFSTTGNLTVHQRTHTHPRAPIPKPFQYKCPECGKSFSQAGHLASHQRTHTGEKPYKCPECGKSFSHTGH | |
| | g | | LLEHQRTHTGEKPYKCPECGKSFSRSDELVRHQRTHTHPRAPIPKPFQYKCPECGKSFSRSDELVRHQRTH | |
| | | | TGKKTS | |
| + | gatgagTgtgT | 184 | LEPGEKPYKCPECGKSFSSKKALTEHQRTHTPNPHRRTDPSHKPFQYKCPECGKSFSQKSSLIAHQRTHTG | 666 |
| | gcgctgataAT | | EKPYKCPECGKSFSRNDALTEHQRTHTGEKPYKCPECGKSFSRSDDLVRHQRTHTHPRAPIPKPFQYKCPE | |
| | cac | | CGKSFSRSDELVRHQRTHTHPRAPIPKPFQYKCPECGKSFSRSDNLVRHQRTHTGEKPYKCPECGKSFSTS | |
| | | | GNLVRHQRTHTGKKTS | |
| + | gagTgtgTgc | 185 | LEPGEKPYKCPECGKSFSRSDKLVRHQRTHTGEKPYKCPECGKSFSSKKALTEHQRTHTPNPHRRTDPSH | 667 |
| | gctgataATca | | KPFQYKCPECGKSFSQKSSLIAHQRTHTGEKPYKCPECGKSFSRNDALTEHQRTHTGEKPYKCPECGKSFS | |
| | cggg | | RSDDLVRHQRTHTHPRAPIPKPFQYKCPECGKSFSRSDELVRHQRTHTHPRAPIPKPFQYKCPECGKSFSRS | |
| | | | DNLVRHQRTHTGKKTS | |
| + | atgagtgtgTg | 186 | LEPGEKPYKCPECGKSFSSKKALTEHQRTHTPNPHRRTDPSHKPFQYKCPECGKSFSQKSSLIAHQRTHTG | 668 |
| | cgctgataATc | | EKPYKCPECGKSFSRNDALTEHQRTHTGEKPYKCPECGKSFSRSDDLVRHQRTHTHPRAPIPKPFQYKCPE | |
| | ac | | CGKSFSRSDELVRHQRTHTGEKPYKCPECGKSFSHRTTLTNHQRTHTGEKPYKCPECGKSFSRRDELNVH | |
| | | | QRTHTGKKTS | |
| + | agtgtgTgcgc | 187 | LEPGEKPYKCPECGKSFSRSDKLVRHQRTHTGEKPYKCPECGKSFSSKKALTEHQRTHTPNPHRRTDPSH | 669 |
| | tgataATcacg | | KPFQYKCPECGKSFSQKSSLIAHQRTHTGEKPYKCPECGKSFSRNDALTEHQRTHTGEKPYKCPECGKSFS | |
| | gg | | RSDDLVRHQRTHTHPRAPIPKPFQYKCPECGKSFSRSDELVRHQRTHTGEKPYKCPECGKSFSHRTTLTNH | |
| | | | QRTHTGKKTS | |
| + | gtgTgcgctga | 188 | LEPGEKPYKCPECGKSFSRSDELVRHQRTHTGEKPYKCPECGKSFSRSDKLVRHQRTHTGEKPYKCPECG | 670 |
| | taATcacggg | | KSFSSKKALTEHQRTHTPNPHRRTDPSHKPFQYKCPECGKSFSQKSSLIAHQRTHTGEKPYKCPECGKSFS | |
| | gtg | | RNDALTEHQRTHTGEKPYKCPECGKSFSRSDDLVRHQRTHTHPRAPIPKPFQYKCPECGKSFSRSDELVRH | |
| | | | QRTHTGKKTS | |
| + | gtgcgctgaTa | 189 | LEPGEKPYKCPECGKSFSRSDELVRHQRTHTGEKPYKCPECGKSFSRSDKLVRHQRTHTGEKPYKCPECG | 671 |
| | atcacggggtg | | KSFSSKKALTEHQRTHTGEKPYKCPECGKSFSTTGNLTVHQRTHTHPRAPIPKPFQYKCPECGKSFSQAGH | |
| | | | LASHQRTHTGEKPYKCPECGKSFSHTGHLLEHQRTHTGEKPYKCPECGKSFSRSDELVRHQRTHTGKKTS | |
| + | cgctgaTaatc | 190 | LEPGEKPYKCPECGKSFSRSDKLVRHQRTHTGEKPYKCPECGKSFSRSDELVRHQRTHTGEKPYKCPECG | 672 |
| | acggggtgggg | | KSFSRSDKLVRHQRTHTGEKPYKCPECGKSFSSKKALTEHQRTHTGEKPYKCPECGKSFSTTGNLTVHQR | |
| | | | THTHPRAPIPKPFQYKCPECGKSFSQAGHLASHQRTHTGEKPYKCPECGKSFSHTGHLLEHQRTHTGKKT | |
| | | | S | |
| + | tgaTaatcacg | 191 | LEPGEKPYKCPECGKSFSRSDKLVRHQRTHTGEKPYKCPECGKSFSRSDKLVRHQRTHTGEKPYKCPECG | 673 |
| | gggtgggggg | | KSFSRSDELVRHQRTHTGEKPYKCPECGKSFSRSDKLVRHQRTHTGEKPYKCPECGKSFSSKKALTEHQR | |
| | g | | THTGEKPYKCPECGKSFSTTGNLTVHQRTHTHPRAPIPKPFQYKCPECGKSFSQAGHLASHQRTHTGKKT | |
| | | | S | |
| + | gcgctgataAT | 192 | LEPGEKPYKCPECGKSFSRSDKLVRHQRTHTGEKPYKCPECGKSFSRSDELVRHQRTHTGEKPYKCPECG | 674 |
| | cacggggtggg | | KSFSRSDKLVRHQRTHTGEKPYKCPECGKSFSSKKALTEHQRTHTPNPHRRTDPSHKPFQYKCPECGKSFS | |
| | g | | QKSSLIAHQRTHTGEKPYKCPECGKSFSRNDALTEHQRTHTGEKPYKCPECGKSFSRSDDLVRHQRTHTG | |
| | | | KKTS | |
| + | ctgataATcac | 193 | LEPGEKPYKCPECGKSFSRSDKLVRHQRTHTGEKPYKCPECGKSFSRSDKLVRHQRTHTGEKPYKCPECG | 675 |
| | ggggtggggg | | KSFSRSDELVRHQRTHTGEKPYKCPECGKSFSRSDKLVRHQRTHTGEKPYKCPECGKSFSSKKALTEHQR | |
| | gg | | THTPNPHRRTDPSHKPFQYKCPECGKSFSQKSSLIAHQRTHTGEKPYKCPECGKSFSRNDALTEHQRTHTG | |
| | | | KKTS | |
| + | ataATcacgg | 194 | LEPGEKPYKCPECGKSFSTSGHLVRHQRTHTGEKPYKCPECGKSFSRSDKLVRHQRTHTGEKPYKCPECG | 676 |
| | ggtggggggg | | KSFSRSDKLVRHQRTHTGEKPYKCPECGKSFSRSDELVRHQRTHTGEKPYKCPECGKSFSRSDKLVRHQR | |
| | ggt | | THTGEKPYKCPECGKSFSSKKALTEHQRTHTPNPHRRTDPSHKPFQYKCPECGKSFSQKSSLIAHQRTHTG | |
| | | | KKTS | |
| + | gctgataatcac | 195 | LEPGEKPYKCPECGKSFSRSDKLVRHQRTHTGEKPYKCPECGKSFSRSDELVRHQRTHTGEKPYKCPECG | 677 |
| | ggggtgggg | | KSFSRSDKLVRHQRTHTGEKPYKCPECGKSFSSKKALTEHQRTHTGEKPYKCPECGKSFSTTGNLTVHQR | |
| | | | THTGEKPYKCPECGKSFSTSGNLVRHQRTHTGEKPYKCPECGKSFSTSGELVRHQRTHTGKKTS | |
| + | gataatcacggg | 196 | LEPGEKPYKCPECGKSFSRSDKLVRHQRTHTGEKPYKCPECGKSFSRSDKLVRHQRTHTGEKPYKCPECG | 678 |
| | gtggggggg | | KSFSRSDELVRHQRTHTGEKPYKCPECGKSFSRSDKLVRHQRTHTGEKPYKCPECGKSFSSKKALTEHQR | |
| | | | THTGEKPYKCPECGKSFSTTGNLTVHQRTHTGEKPYKCPECGKSFSTSGNLVRHQRTHTGKKTS | |
| + | aatcacggggt | 197 | LEPGEKPYKCPECGKSFSTSGHLVRHQRTHTGEKPYKCPECGKSFSRSDKLVRHQRTHTGEKPYKCPECG | 679 |
| | gggggggggt | | KSFSRSDKLVRHQRTHTGEKPYKCPECGKSFSRSDELVRHQRTHTGEKPYKCPECGKSFSRSDKLVRHQR | |
| | | | THTGEKPYKCPECGKSFSSKKALTEHQRTHTGEKPYKCPECGKSFSTTGNLTVHQRTHTGKKTS | |
| + | acggggtgggg | 198 | LEPGEKPYKCPECGKSFSTSGNLTEHQRTHTPNPHRRTDPSHKPFQYKCPECGKSFSTSGSLVRHQRTHTG | 680 |
| | gggggttCTc | | EKPYKCPECGKSFSRSDKLVRHQRTHTGEKPYKCPECGKSFSRSDKLVRHQRTHTGEKPYKCPECGKSFS | |
| | at | | RSDHLTTHQRTHTGEKPYKCPECGKSFSRSDKLVRHQRTHTGEKPYKCPECGKSFSRTDTLRDHQRTHTG | |
| | | | KKTS | |
| + | gggtgggggg | 199 | LEPGEKPYKCPECGKSFSHRTTLTNHQRTHTGEKPYKCPECGKSFSTSGNLTEHQRTHTPNPHRRTDPSHK | 681 |
| | gggttCTcata | | PFQYKCPECGKSFSTSGSLVRHQRTHTGEKPYKCPECGKSFSRSDKLVRHQRTHTGEKPYKCPECGKSFSR | |
| | gt | | SDKLVRHQRTHTGEKPYKCPECGKSFSRSDHLTTHQRTHTGEKPYKCPECGKSFSRSDKLVRHQRTHTGK | |
| | | | KTS | |
| + | cttTTcttgatT | 200 | LEPGEKPYKCPECGKSFSQRANLRAHQRTHTGEKPYKCPECGKSFSTTGALTEHQRTHTGEKPYKCPECG | 682 |
| | atgagacttaaa | | KSFSQLAHLRAHQRTHTGEKPYKCPECGKSFSRRDELNVHQRTHTHPRAPIPKPFQYKCPECGKSFSTSGN | |
| | | | LVRHQRTHTGEKPYKCPECGKSFSTTGALTEHQRTHTPNPHRRTDPSHKPFQYKCPECGKSFSTTGALTE | |
| | | | HQRTHTGKKTS | |
| + | cttgatTatgag | 201 | LEPGEKPYKCPECGKSFSRSDKLTEHQRTHTGEKPYKCPECGKSFSQRANLRAHQRTHTGEKPYKCPECG | 683 |
| | acttaaacgg | | KSFSTTGALTEHQRTHTGEKPYKCPECGKSFSQLAHLRAHQRTHTGEKPYKCPECGKSFSRRDELNVHQR | |
| | | | THTHPRAPIPKPFQYKCPECGKSFSTSGNLVRHQRTHTGEKPYKCPECGKSFSTTGALTEHQRTHTGKKTS | |
| + | gatTatgagact | 202 | LEPGEKPYKCPECGKSFSQRANLRAHQRTHTGEKPYKCPECGKSFSRSDKLTEHQRTHTGEKPYKCPECG | 684 |
| | taaacggaaa | | KSFSQRANLRAHQRTHTGEKPYKCPECGKSFSTTGALTEHQRTHTGEKPYKCPECGKSFSQLAHLRAHQR | |
| | | | THTGEKPYKCPECGKSFSRRDELNVHQRTHTHPRAPIPKPFQYKCPECGKSFSTSGNLVRHQRTHTGKKTS | |
| + | tgaTTatgaga | 203 | LEPGEKPYKCPECGKSFSQRANLRAHQRTHTGEKPYKCPECGKSFSRSDKLTEHQRTHTGEKPYKCPECG | 685 |
| | cttaaacggaaa | | KSFSQRANLRAHQRTHTGEKPYKCPECGKSFSTTGALTEHQRTHTGEKPYKCPECGKSFSQLAHLRAHQR | |
| | | | THTGEKPYKCPECGKSFSRRDELNVHQRTHTPNPHRRTDPSHKPFQYKCPECGKSFSQAGHLASHQRTHT | |
| | | | GKKTS | |
| + | attatgagactta | 204 | LEPGEKPYKCPECGKSFSQRANLRAHQRTHTGEKPYKCPECGKSFSRSDKLTEHQRTHTGEKPYKCPECG | 686 |
| | aacggaaa | | KSFSQRANLRAHQRTHTGEKPYKCPECGKSFSTTGALTEHQRTHTGEKPYKCPECGKSFSQLAHLRAHQR | |
| | | | THTGEKPYKCPECGKSFSRRDELNVHQRTHTGEKPYKCPECGKSFSHKNALQNHQRTHTGKKTS | |
| + | aacggaaatTT | 205 | LEPGEKPYKCPECGKSFSTSGSLVRHQRTHTGEKPYKCPECGKSFSRSDHLTTHQRTHTPNPHRRTDPSHK | 687 |
| | tgaaatTTtgg | | PFQYKCPECGKSFSTTGNLTVHQRTHTGEKPYKCPECGKSFSQAGHLASHQRTHTPNPHRRTDPSHKPFQ | |
| | gtt | | YKCPECGKSFSTTGNLTVHQRTHTGEKPYKCPECGKSFSQRAHLERHQRTHTGEKPYKCPECGKSFSDSG | |
| | | | NLRVHQRTHTGKKTS | |
| + | acggaaattTtg | 206 | LEPGEKPYKCPECGKSFSTSGSLVRHQRTHTGEKPYKCPECGKSFSRSDHLTTHQRTHTPNPHRRTDPSHK | 688 |
| | aaatTTtgggtt | | PFQYKCPECGKSFSTTGNLTVHQRTHTGEKPYKCPECGKSFSQAGHLASHQRTHTHPRAPIPKPFQYKCPE | |
| | | | CGKSFSHKNALQNHQRTHTGEKPYKCPECGKSFSQSSNLVRHQRTHTGEKPYKCPECGKSFSRTDTLRDH | |
| | | | QRTHTGKKTS | |
| + | cttTgcccttTa | 207 | LEPGEKPYKCPECGKSFSRNDALTEHQRTHTGEKPYKCPECGKSFSTSGNLTEHQRTHTGEKPYKCPECG | 689 |
| | cgagtcatctg | | KSFSHRTTLTNHQRTHTGEKPYKCPECGKSFSRTDTLRDHQRTHTHPRAPIPKPFQYKCPECGKSFSTTGA | |
| | | | LTEHQRTHTGEKPYKCPECGKSFSDCRDLARHQRTHTHPRAPIPKPFQYKCPECGKSFSTTGALTEHQRTH | |
| | | | TGKKTS | |
| + | gcccttTacga | 208 | LEPGEKPYKCPECGKSFSQRANLRAHQRTHTGEKPYKCPECGKSFSRNDALTEHQRTHTGEKPYKCPECG | 690 |
| | gtcatctgaaa | | KSFSTSGNLTEHQRTHTGEKPYKCPECGKSFSHRTTLTNHQRTHTGEKPYKCPECGKSFSRTDTLRDHQR | |
| | | | THTHPRAPIPKPFQYKCPECGKSFSTTGALTEHQRTHTGEKPYKCPECGKSFSDCRDLARHQRTHTGKKTS | |
| + | cttTacgagtca | 209 | LEPGEKPYKCPECGKSFSQKSSLIAHQRTHTGEKPYKCPECGKSFSQRANLRAHQRTHTGEKPYKCPECG | 691 |
| | tctgaaaata | | KSFSRNDALTEHQRTHTGEKPYKCPECGKSFSTSGNLTEHQRTHTGEKPYKCPECGKSFSHRTTLTNHQR | |
| | | | THTGEKPYKCPECGKSFSRTDTLRDHQRTHTHPRAPIPKPFQYKCPECGKSFSTTGALTEHQRTHTGKKTS | |
| + | cctTTacgagt | 210 | LEPGEKPYKCPECGKSFSQKSSLIAHQRTHTGEKPYKCPECGKSFSQRANLRAHQRTHTGEKPYKCPECG | 692 |
| | catctgaaaata | | KSFSRNDALTEHQRTHTGEKPYKCPECGKSFSTSGNLTEHQRTHTGEKPYKCPECGKSFSHRTTLTNHQR | |
| | | | THTGEKPYKCPECGKSFSRTDTLRDHQRTHTPNPHRRTDPSHKPFQYKCPECGKSFSTKNSLTEHQRTHT | |
| | | | GKKTS | |
| + | acgagtcatctg | 211 | LEPGEKPYKCPECGKSFSQAGHLASHQRTHTGEKPYKCPECGKSFSQKSSLIAHQRTHTGEKPYKCPECG | 693 |
| | aaaatatga | | KSFSQRANLRAHQRTHTGEKPYKCPECGKSFSRNDALTEHQRTHTGEKPYKCPECGKSFSTSGNLTEHQR | |
| | | | THTGEKPYKCPECGKSFSHRTTLTNHQRTHTGEKPYKCPECGKSFSRTDTLRDHQRTHTGKKTS | |
| + | tgaaaaTatgat | 212 | LEPGEKPYKCPECGKSFSSKKALTEHQRTHTPNPHRRTDPSHKPFQYKCPECGKSFSSKKHLAEHQRTHT | 694 |
| | tTcttcccCTc | | GEKPYKCPECGKSFSTTGALTEHQRTHTHPRAPIPKPFQYKCPECGKSFSHKNALQNHQRTHTGEKPYKC | |
| | ac | | PECGKSFSRRDELNVHQRTHTHPRAPIPKPFQYKCPECGKSFSQRANLRAHQRTHTGEKPYKCPECGKSFS | |
| | | | QAGHLASHQRTHTGKKTS | |
| + | aaaTatgattTc | 213 | LEPGEKPYKCPECGKSFSSKKALTEHQRTHTGEKPYKCPECGKSFSSKKALTEHQRTHTPNPHRRTDPSHK | 695 |
| | ttcccCTcacc | | PFQYKCPECGKSFSSKKHLAEHQRTHTGEKPYKCPECGKSFSTTGALTEHQRTHTHPRAPIPKPFQYKCPE | |
| | ac | | CGKSFSHKNALQNHQRTHTGEKPYKCPECGKSFSRRDELNVHQRTHTHPRAPIPKPFQYKCPECGKSFSQ | |
| | | | RANLRAHQRTHTGKKTS | |
| + | gaaaatatgatt | 214 | LEPGEKPYKCPECGKSFSSKKALTEHQRTHTPNPHRRTDPSHKPFQYKCPECGKSFSSKKHLAEHQRTHT | 696 |
| | TcttcccCTca | | GEKPYKCPECGKSFSTTGALTEHQRTHTHPRAPIPKPFQYKCPECGKSFSHKNALQNHQRTHTGEKPYKC | |
| | c | | PECGKSFSRRDELNVHQRTHTGEKPYKCPECGKSFSTTGNLTVHQRTHTGEKPYKCPECGKSFSQSSNLV | |
| | | | RHQRTHTGKKTS | |
| + | aatatgattTctt | 215 | LEPGEKPYKCPECGKSFSSKKALTEHQRTHTGEKPYKCPECGKSFSSKKALTEHQRTHTPNPHRRTDPSHK | 697 |
| | cccCTcacca | | PFQYKCPECGKSFSSKKHLAEHQRTHTGEKPYKCPECGKSFSTTGALTEHQRTHTHPRAPIPKPFQYKCPE | |
| | c | | CGKSFSHKNALQNHQRTHTGEKPYKCPECGKSFSRRDELNVHQRTHTGEKPYKCPECGKSFSTTGNLTV | |
| | | | HQRTHTGKKTS | |
| + | atgattTcttccc | 216 | LEPGEKPYKCPECGKSFSQLAHLRAHQRTHTGEKPYKCPECGKSFSSKKALTEHQRTHTGEKPYKCPECG | 698 |
| | CTcaccacag | | KSFSSKKALTEHQRTHTPNPHRRTDPSHKPFQYKCPECGKSFSSKKHLAEHQRTHTGEKPYKCPECGKSFS | |
| | a | | TTGALTEHQRTHTHPRAPIPKPFQYKCPECGKSFSHKNALQNHQRTHTGEKPYKCPECGKSFSRRDELNV | |
| | | | HQRTHTGKKTS | |
| + | attTcttcccCT | 217 | LEPGEKPYKCPECGKSFSTSGHLVRHQRTHTGEKPYKCPECGKSFSQLAHLRAHQRTHTGEKPYKCPECG | 699 |
| | caccacagagg | | KSFSSKKALTEHQRTHTGEKPYKCPECGKSFSSKKALTEHQRTHTPNPHRRTDPSHKPFQYKCPECGKSFS | |
| | t | | SKKHLAEHQRTHTGEKPYKCPECGKSFSTTGALTEHQRTHTHPRAPIPKPFQYKCPECGKSFSHKNALQN | |
| | | | HQRTHTGKKTS | |
| + | gatTTcttccc | 218 | LEPGEKPYKCPECGKSFSTSGHLVRHQRTHTGEKPYKCPECGKSFSQLAHLRAHQRTHTGEKPYKCPECG | 700 |
| | CTcaccacag | | KSFSSKKALTEHQRTHTGEKPYKCPECGKSFSSKKALTEHQRTHTPNPHRRTDPSHKPFQYKCPECGKSFS | |
| | aggt | | SKKHLAEHQRTHTGEKPYKCPECGKSFSTTGALTEHQRTHTPNPHRRTDPSHKPFQYKCPECGKSFSTSG | |
| | | | NLVRHQRTHTGKKTS | |
| + | cttcccCTcac | 219 | LEPGEKPYKCPECGKSFSRSDNLVRHQRTHTGEKPYKCPECGKSFSTSGHLVRHQRTHTGEKPYKCPECG | 701 |
| | cacagaggtga | | KSFSQLAHLRAHQRTHTGEKPYKCPECGKSFSSKKALTEHQRTHTGEKPYKCPECGKSFSSKKALTEHQR | |
| | g | | THTPNPHRRTDPSHKPFQYKCPECGKSFSSKKHLAEHQRTHTGEKPYKCPECGKSFSTTGALTEHQRTHT | |
| | | | GKKTS | |
| + | cccCTcacca | 220 | LEPGEKPYKCPECGKSFSRSDHLTNHQRTHTGEKPYKCPECGKSFSRSDNLVRHQRTHTGEKPYKCPECG | 702 |
| | cagaggtgaga | | KSFSTSGHLVRHQRTHTGEKPYKCPECGKSFSQLAHLRAHQRTHTGEKPYKCPECGKSFSSKKALTEHQR | |
| | 88 | | THTGEKPYKCPECGKSFSSKKALTEHQRTHTPNPHRRTDPSHKPFQYKCPECGKSFSSKKHLAEHQRTHT | |
| | | | GKKTS | |
| + | cccTcaccaca | 221 | LEPGEKPYKCPECGKSFSRSDHLTNHQRTHTGEKPYKCPECGKSFSRSDNLVRHQRTHTGEKPYKCPECG | 703 |
| | gaggtgagagg | | KSFSTSGHLVRHQRTHTGEKPYKCPECGKSFSQLAHLRAHQRTHTGEKPYKCPECGKSFSSKKALTEHQR | |
| | | | THTGEKPYKCPECGKSFSSKKALTEHQRTHTHPRAPIPKPFQYKCPECGKSFSSKKHLAEHQRTHTGKKTS | |
| + | cctcaccacaga | 222 | LEPGEKPYKCPECGKSFSRSDHLTNHQRTHTGEKPYKCPECGKSFSRSDNLVRHQRTHTGEKPYKCPECG | 704 |
| | ggtgagagg | | KSFSTSGHLVRHQRTHTGEKPYKCPECGKSFSQLAHLRAHQRTHTGEKPYKCPECGKSFSSKKALTEHQR | |
| | | | THTGEKPYKCPECGKSFSSKKALTEHQRTHTGEKPYKCPECGKSFSTKNSLTEHQRTHTGKKTS | |
| + | accacagaggt | 223 | LEPGEKPYKCPECGKSFSQSGNLTEHQRTHTPNPHRRTDPSHKPFQYKCPECGKSFSTSGHLVRHQRTHTG | 705 |
| | gagaggtATc | | EKPYKCPECGKSFSQLAHLRAHQRTHTGEKPYKCPECGKSFSRSDELVRHQRTHTGEKPYKCPECGKSFS | |
| | aa | | RSDNLVRHQRTHTGEKPYKCPECGKSFSSPADLTRHQRTHTGEKPYKCPECGKSFSDKKDLTRHQRTHTG | |
| | | | KKTS | |
| + | acagaggtgag | 224 | LEPGEKPYKCPECGKSFSQAGHLASHQRTHTGEKPYKCPECGKSFSQSGNLTEHQRTHTPNPHRRTDPSH | 706 |
| | aggtATcaatg | | KPFQYKCPECGKSFSTSGHLVRHQRTHTGEKPYKCPECGKSFSQLAHLRAHQRTHTGEKPYKCPECGKSF | |
| | a | | SRSDELVRHQRTHTGEKPYKCPECGKSFSRSDNLVRHQRTHTGEKPYKCPECGKSFSSPADLTRHQRTHT | |
| | | | GKKTS | |
| + | gaggtgagagg | 225 | LEPGEKPYKCPECGKSFSTSGNLVRHQRTHTGEKPYKCPECGKSFSQAGHLASHQRTHTGEKPYKCPECG | 707 |
| | tATcaatgaga | | KSFSQSGNLTEHQRTHTPNPHRRTDPSHKPFQYKCPECGKSFSTSGHLVRHQRTHTGEKPYKCPECGKSFS | |
| | t | | QLAHLRAHQRTHTGEKPYKCPECGKSFSRSDELVRHQRTHTGEKPYKCPECGKSFSRSDNLVRHQRTHT | |
| | | | GKKTS | |
| + | gtgagaggtA | 226 | LEPGEKPYKCPECGKSFSTTGNLTVHQRTHTGEKPYKCPECGKSFSTSGNLVRHQRTHTGEKPYKCPECG | 708 |
| | Tcaatgagata | | KSFSQAGHLASHQRTHTGEKPYKCPECGKSFSQSGNLTEHQRTHTPNPHRRTDPSHKPFQYKCPECGKSF | |
| | at | | STSGHLVRHQRTHTGEKPYKCPECGKSFSQLAHLRAHQRTHTGEKPYKCPECGKSFSRSDELVRHQRTHT | |
| | | | GKKTS | |
| + | agaggtATca | 227 | LEPGEKPYKCPECGKSFSRSDHLTNHQRTHTGEKPYKCPECGKSFSTTGNLTVHQRTHTGEKPYKCPECG | 709 |
| | atgagataatag | | KSFSTSGNLVRHQRTHTGEKPYKCPECGKSFSQAGHLASHQRTHTGEKPYKCPECGKSFSQSGNLTEHQR | |
| | g | | THTPNPHRRTDPSHKPFQYKCPECGKSFSTSGHLVRHQRTHTGEKPYKCPECGKSFSQLAHLRAHQRTHT | |
| | | | GKKTS | |
| + | ggtATcaatga | 228 | LEPGEKPYKCPECGKSFSTSGELVRHQRTHTGEKPYKCPECGKSFSRSDHLTNHQRTHTGEKPYKCPECG | 710 |
| | gataatagggct | | KSFSTTGNLTVHQRTHTGEKPYKCPECGKSFSTSGNLVRHQRTHTGEKPYKCPECGKSFSQAGHLASHQR | |
| | | | THTGEKPYKCPECGKSFSQSGNLTEHQRTHTPNPHRRTDPSHKPFQYKCPECGKSFSTSGHLVRHQRTHT | |
| | | | GKKTS | |
| + | ccacagaggtg | 229 | LEPGEKPYKCPECGKSFSQSGNLTEHQRTHTHPRAPIPKPFQYKCPECGKSFSQSSSLVRHQRTHTGEKPY | 711 |
| | agaggtaTcaa | | KCPECGKSFSRSDNLVRHQRTHTGEKPYKCPECGKSFSQAGHLASHQRTHTGEKPYKCPECGKSFSRSDH | |
| | | | LTNHQRTHTGEKPYKCPECGKSFSRADNLTEHQRTHTGEKPYKCPECGKSFSTSHSLTEHQRTHTGKKTS | |
| + | cagaggtgaga | 230 | LEPGEKPYKCPECGKSFSQAGHLASHQRTHTGEKPYKCPECGKSFSQSGNLTEHQRTHTHPRAPIPKPFQY | 712 |
| | ggtaTcaatga | | KCPECGKSFSQSSSLVRHQRTHTGEKPYKCPECGKSFSRSDNLVRHQRTHTGEKPYKCPECGKSFSQAGH | |
| | | | LASHQRTHTGEKPYKCPECGKSFSRSDHLTNHQRTHTGEKPYKCPECGKSFSRADNLTEHQRTHTGKKTS | |
| + | aggtgagaggt | 231 | LEPGEKPYKCPECGKSFSTSGNLVRHQRTHTGEKPYKCPECGKSFSQAGHLASHQRTHTGEKPYKCPECG | 713 |
| | aTcaatgagat | | KSFSQSGNLTEHQRTHTHPRAPIPKPFQYKCPECGKSFSQSSSLVRHQRTHTGEKPYKCPECGKSFSRSDN | |
| | | | LVRHQRTHTGEKPYKCPECGKSFSQAGHLASHQRTHTGEKPYKCPECGKSFSRSDHLTNHQRTHTGKKTS | |
| + | tgagaggtaTc | 232 | LEPGEKPYKCPECGKSFSTTGNLTVHQRTHTGEKPYKCPECGKSFSTSGNLVRHQRTHTGEKPYKCPECG | 714 |
| | aatgagataat | | KSFSQAGHLASHQRTHTGEKPYKCPECGKSFSQSGNLTEHQRTHTHPRAPIPKPFQYKCPECGKSFSQSSS | |
| | | | LVRHQRTHTGEKPYKCPECGKSFSRSDNLVRHQRTHTGEKPYKCPECGKSFSQAGHLASHQRTHTGKKT | |
| | | | S | |
| + | gaggtaTcaat | 233 | LEPGEKPYKCPECGKSFSRSDHLTNHQRTHTGEKPYKCPECGKSFSTTGNLTVHQRTHTGEKPYKCPECG | 715 |
| | gagataatagg | | KSFSTSGNLVRHQRTHTGEKPYKCPECGKSFSQAGHLASHQRTHTGEKPYKCPECGKSFSQSGNLTEHQR | |
| | | | THTHPRAPIPKPFQYKCPECGKSFSQSSSLVRHQRTHTGEKPYKCPECGKSFSRSDNLVRHQRTHTGKKTS | |
| + | gtaTcaatgag | 234 | LEPGEKPYKCPECGKSFSTSGELVRHQRTHTGEKPYKCPECGKSFSRSDHLTNHQRTHTGEKPYKCPECG | 716 |
| | ataatagggct | | KSFSTTGNLTVHQRTHTGEKPYKCPECGKSFSTSGNLVRHQRTHTGEKPYKCPECGKSFSQAGHLASHQR | |
| | | | THTGEKPYKCPECGKSFSQSGNLTEHQRTHTHPRAPIPKPFQYKCPECGKSFSQSSSLVRHQRTHTGKKTS | |
| + | caatgagataat | 235 | LEPGEKPYKCPECGKSFSTSGNLTEHQRTHTGEKPYKCPECGKSFSTSGELVRHQRTHTGEKPYKCPECG | 717 |
| | agggctcat | | KSFSRSDHLTNHQRTHTGEKPYKCPECGKSFSTTGNLTVHQRTHTGEKPYKCPECGKSFSTSGNLVRHQR | |
| | | | THTGEKPYKCPECGKSFSQAGHLASHQRTHTGEKPYKCPECGKSFSQSGNLTEHQRTHTGKKTS | |
| + | tgagataatagg | 236 | LEPGEKPYKCPECGKSFSRSDNLVRHQRTHTGEKPYKCPECGKSFSTSGNLTEHQRTHTGEKPYKCPECG | 718 |
| | gctcatgag | | KSFSTSGELVRHQRTHTGEKPYKCPECGKSFSRSDHLTNHQRTHTGEKPYKCPECGKSFSTTGNLTVHQR | |
| | | | THTGEKPYKCPECGKSFSTSGNLVRHQRTHTGEKPYKCPECGKSFSQAGHLASHQRTHTGKKTS | |
| + | gataatagggct | 237 | LEPGEKPYKCPECGKSFSQRANLRAHQRTHTGEKPYKCPECGKSFSRSDNLVRHQRTHTGEKPYKCPECG | 719 |
| | catgagaaa | | KSFSTSGNLTEHQRTHTGEKPYKCPECGKSFSTSGELVRHQRTHTGEKPYKCPECGKSFSRSDHLTNHQRT | |
| | | | HTGEKPYKCPECGKSFSTTGNLTVHQRTHTGEKPYKCPECGKSFSTSGNLVRHQRTHTGKKTS | |
| + | aatagggctcat | 238 | LEPGEKPYKCPECGKSFSTSHSLTEHQRTHTGEKPYKCPECGKSFSQRANLRAHQRTHTGEKPYKCPECG | 720 |
| | gagaaacca | | KSFSRSDNLVRHQRTHTGEKPYKCPECGKSFSTSGNLTEHQRTHTGEKPYKCPECGKSFSTSGELVRHQR | |
| | | | THTGEKPYKCPECGKSFSRSDHLTNHQRTHTGEKPYKCPECGKSFSTTGNLTVHQRTHTGKKTS | |
| + | agggctcatga | 239 | LEPGEKPYKCPECGKSFSRADNLTEHQRTHTGEKPYKCPECGKSFSTSHSLTEHQRTHTGEKPYKCPECG | 721 |
| | gaaaccacag | | KSFSQRANLRAHQRTHTGEKPYKCPECGKSFSRSDNLVRHQRTHTGEKPYKCPECGKSFSTSGNLTEHQR | |
| | | | THTGEKPYKCPECGKSFSTSGELVRHQRTHTGEKPYKCPECGKSFSRSDHLTNHQRTHTGKKTS | |
| + | aatgagataata | 240 | LEPGEKPYKCPECGKSFSRSDNLVRHQRTHTGEKPYKCPECGKSFSTSGNLTEHQRTHTPNPHRRTDPSHK | 722 |
| | gggCTcatga | | PFQYKCPECGKSFSRSDKLVRHQRTHTGEKPYKCPECGKSFSQKSSLIAHQRTHTGEKPYKCPECGKSFSQ | |
| | g | | KSSLIAHQRTHTGEKPYKCPECGKSFSRSDNLVRHQRTHTGEKPYKCPECGKSFSTTGNLTVHQRTHTGK | |
| | | | KTS | |
| + | gagataatagg | 241 | LEPGEKPYKCPECGKSFSQRANLRAHQRTHTGEKPYKCPECGKSFSRSDNLVRHQRTHTGEKPYKCPECG | 723 |
| | gCTcatgaga | | KSFSTSGNLTEHQRTHTPNPHRRTDPSHKPFQYKCPECGKSFSRSDKLVRHQRTHTGEKPYKCPECGKSFS | |
| | aa | | QKSSLIAHQRTHTGEKPYKCPECGKSFSQKSSLIAHQRTHTGEKPYKCPECGKSFSRSDNLVRHQRTHTGK | |
| | | | KTS | |
| + | ataatagggCT | 242 | LEPGEKPYKCPECGKSFSTSHSLTEHQRTHTGEKPYKCPECGKSFSQRANLRAHQRTHTGEKPYKCPECG | 724 |
| | catgagaaacc | | KSFSRSDNLVRHQRTHTGEKPYKCPECGKSFSTSGNLTEHQRTHTPNPHRRTDPSHKPFQYKCPECGKSFS | |
| | a | | RSDKLVRHQRTHTGEKPYKCPECGKSFSQKSSLIAHQRTHTGEKPYKCPECGKSFSQKSSLIAHQRTHTGK | |
| | | | KTS | |
| + | atagggCTcat | 243 | LEPGEKPYKCPECGKSFSRADNLTEHQRTHTGEKPYKCPECGKSFSTSHSLTEHQRTHTGEKPYKCPECG | 725 |
| | gagaaaccaca | | KSFSQRANLRAHQRTHTGEKPYKCPECGKSFSRSDNLVRHQRTHTGEKPYKCPECGKSFSTSGNLTEHQR | |
| | g | | THTPNPHRRTDPSHKPFQYKCPECGKSFSRSDKLVRHQRTHTGEKPYKCPECGKSFSQKSSLIAHQRTHTG | |
| | | | KKTS | |
| + | atgagaTaata | 244 | LEPGEKPYKCPECGKSFSRSDNLVRHQRTHTGEKPYKCPECGKSFSTSGNLTEHQRTHTGEKPYKCPECG | 726 |
| | gggctcatgag | | KSFSTSGELVRHQRTHTGEKPYKCPECGKSFSRSDHLTNHQRTHTGEKPYKCPECGKSFSTTGNLTVHQR | |
| | | | THTHPRAPIPKPFQYKCPECGKSFSQLAHLRAHQRTHTGEKPYKCPECGKSFSRRDELNVHQRTHTGKKT | |
| | | | S | |
| + | agaTaataggg | 245 | LEPGEKPYKCPECGKSFSQRANLRAHQRTHTGEKPYKCPECGKSFSRSDNLVRHQRTHTGEKPYKCPECG | 727 |
| | ctcatgagaaa | | KSFSTSGNLTEHQRTHTGEKPYKCPECGKSFSTSGELVRHQRTHTGEKPYKCPECGKSFSRSDHLTNHQRT | |
| | | | HTGEKPYKCPECGKSFSTTGNLTVHQRTHTHPRAPIPKPFQYKCPECGKSFSQLAHLRAHQRTHTGKKTS | |
| + | tagggcTcatg | 246 | LEPGEKPYKCPECGKSFSRADNLTEHQRTHTGEKPYKCPECGKSFSTSHSLTEHQRTHTGEKPYKCPECG | 728 |
| | agaaaccacag | | KSFSQRANLRAHQRTHTGEKPYKCPECGKSFSRSDNLVRHQRTHTGEKPYKCPECGKSFSTSGNLTEHQR | |
| | | | THTHPRAPIPKPFQYKCPECGKSFSDPGHLVRHQRTHTGEKPYKCPECGKSFSREDNLHTHQRTHTGKKT | |
| | | | S | |
| + | aaaacaaaagt | 247 | LEPGEKPYKCPECGKSFSQAGHLASHQRTHTPNPHRRTDPSHKPFQYKCPECGKSFSRSDNLVRHQRTHT | 729 |
| | gTatagagTTt | | GEKPYKCPECGKSFSQKSSLIAHQRTHTHPRAPIPKPFQYKCPECGKSFSRSDELVRHQRTHTGEKPYKCP | |
| | ga | | ECGKSFSQRANLRAHQRTHTGEKPYKCPECGKSFSSPADLTRHQRTHTGEKPYKCPECGKSFSQRANLRA | |
| | | | HQRTHTGKKTS | |
| + | acaaaagtgTa | 248 | LEPGEKPYKCPECGKSFSQRANLRAHQRTHTGEKPYKCPECGKSFSQAGHLASHQRTHTPNPHRRTDPSH | 730 |
| | tagagTTtgaa | | KPFQYKCPECGKSFSRSDNLVRHQRTHTGEKPYKCPECGKSFSQKSSLIAHQRTHTHPRAPIPKPFQYKCP | |
| | aa | | ECGKSFSRSDELVRHQRTHTGEKPYKCPECGKSFSQRANLRAHQRTHTGEKPYKCPECGKSFSSPADLTR | |
| | | | HQRTHTGKKTS | |
| + | aaagtgTatag | 249 | LEPGEKPYKCPECGKSFSQRANLRAHQRTHTGEKPYKCPECGKSFSQRANLRAHQRTHTGEKPYKCPEC | 731 |
| | agTTtgaaaaa | | GKSFSQAGHLASHQRTHTPNPHRRTDPSHKPFQYKCPECGKSFSRSDNLVRHQRTHTGEKPYKCPECGKS | |
| | aa | | FSQKSSLIAHQRTHTHPRAPIPKPFQYKCPECGKSFSRSDELVRHQRTHTGEKPYKCPECGKSFSQRANLR | |
| | | | AHQRTHTGKKTS | |
| + | gtgTatagagT | 250 | LEPGEKPYKCPECGKSFSQRANLRAHQRTHTGEKPYKCPECGKSFSQRANLRAHQRTHTGEKPYKCPEC | 732 |
| | Ttgaaaaaaaa | | GKSFSQRANLRAHQRTHTGEKPYKCPECGKSFSQAGHLASHQRTHTPNPHRRTDPSHKPFQYKCPECGKS | |
| | aa | | FSRSDNLVRHQRTHTGEKPYKCPECGKSFSQKSSLIAHQRTHTHPRAPIPKPFQYKCPECGKSFSRSDELV | |
| | | | RHQRTHTGKKTS | |
| + | aaacaaaagTg | 251 | LEPGEKPYKCPECGKSFSQAGHLASHQRTHTHPRAPIPKPFQYKCPECGKSFSHRTTLTNHQRTHTGEKPY | 733 |
| | tatagagtTtga | | KCPECGKSFSREDNLHTHQRTHTGEKPYKCPECGKSFSQSSSLVRHQRTHTHPRAPIPKPFQYKCPECGKS | |
| | | | FSRKDNLKNHQRTHTGEKPYKCPECGKSFSQSGNLTEHQRTHTGEKPYKCPECGKSFSQRANLRAHQRT | |
| | | | HTGKKTS | |
| + | caaaagTgtat | 252 | LEPGEKPYKCPECGKSFSQRANLRAHQRTHTGEKPYKCPECGKSFSQAGHLASHQRTHTHPRAPIPKPFQ | 734 |
| | agagtTtgaaa | | YKCPECGKSFSHRTTLTNHQRTHTGEKPYKCPECGKSFSREDNLHTHQRTHTGEKPYKCPECGKSFSQSSS | |
| | a | | LVRHQRTHTHPRAPIPKPFQYKCPECGKSFSRKDNLKNHQRTHTGEKPYKCPECGKSFSQSGNLTEHQRT | |
| | | | HTGKKTS | |
| + | aagTgtataga | 253 | LEPGEKPYKCPECGKSFSQRANLRAHQRTHTGEKPYKCPECGKSFSQRANLRAHQRTHTGEKPYKCPEC | 735 |
| | gtTtgaaaaaa | | GKSFSQAGHLASHQRTHTHPRAPIPKPFQYKCPECGKSFSHRTTLTNHQRTHTGEKPYKCPECGKSFSRED | |
| | a | | NLHTHQRTHTGEKPYKCPECGKSFSQSSSLVRHQRTHTHPRAPIPKPFQYKCPECGKSFSRKDNLKNHQR | |
| | | | THTGKKTS | |
| + | aacaaaagtgta | 254 | LEPGEKPYKCPECGKSFSQAGHLASHQRTHTHPRAPIPKPFQYKCPECGKSFSHRTTLTNHQRTHTGEKPY | 736 |
| | tagagtTtga | | KCPECGKSFSREDNLHTHQRTHTGEKPYKCPECGKSFSQSSSLVRHQRTHTGEKPYKCPECGKSFSHRTTL | |
| | | | TNHQRTHTGEKPYKCPECGKSFSQRANLRAHQRTHTGEKPYKCPECGKSFSDSGNLRVHQRTHTGKKTS | |
| + | aaaagtgtatag | 255 | LEPGEKPYKCPECGKSFSQRANLRAHQRTHTGEKPYKCPECGKSFSQAGHLASHQRTHTHPRAPIPKPFQ | 737 |
| | agtTtgaaaa | | YKCPECGKSFSHRTTLTNHQRTHTGEKPYKCPECGKSFSREDNLHTHQRTHTGEKPYKCPECGKSFSQSSS | |
| | | | LVRHQRTHTGEKPYKCPECGKSFSHRTTLTNHQRTHTGEKPYKCPECGKSFSQRANLRAHQRTHTGKKT | |
| | | | S | |
| + | agtgtatagagt | 256 | LEPGEKPYKCPECGKSFSQRANLRAHQRTHTGEKPYKCPECGKSFSQRANLRAHQRTHTGEKPYKCPEC | 738 |
| | Ttgaaaaaaa | | GKSFSQAGHLASHQRTHTHPRAPIPKPFQYKCPECGKSFSHRTTLTNHQRTHTGEKPYKCPECGKSFSRED | |
| | | | NLHTHQRTHTGEKPYKCPECGKSFSQSSSLVRHQRTHTGEKPYKCPECGKSFSHRTTLTNHQRTHTGKKT | |
| | | | S | |
| + | gtatagagtTtg | 257 | LEPGEKPYKCPECGKSFSQRANLRAHQRTHTGEKPYKCPECGKSFSQRANLRAHQRTHTGEKPYKCPEC | 739 |
| | aaaaaaaaaa | | GKSFSQRANLRAHQRTHTGEKPYKCPECGKSFSQAGHLASHQRTHTHPRAPIPKPFQYKCPECGKSFSHR | |
| | | | TTLTNHQRTHTGEKPYKCPECGKSFSREDNLHTHQRTHTGEKPYKCPECGKSFSQSSSLVRHQRTHTGKK | |
| | | | TS | |
| + | tagagtTtgaaa | 258 | LEPGEKPYKCPECGKSFSDSGNLRVHQRTHTGEKPYKCPECGKSFSQRANLRAHQRTHTGEKPYKCPECG | 740 |
| | aaaaaaaaac | | KSFSQRANLRAHQRTHTGEKPYKCPECGKSFSQRANLRAHQRTHTGEKPYKCPECGKSFSQAGHLASHQ | |
| | | | RTHTHPRAPIPKPFQYKCPECGKSFSHRTTLTNHQRTHTGEKPYKCPECGKSFSREDNLHTHQRTHTGKKT | |
| | | | S | |
| + | agtTtgaaaaa | 259 | LEPGEKPYKCPECGKSFSRKDNLKNHQRTHTGEKPYKCPECGKSFSDSGNLRVHQRTHTGEKPYKCPECG | 741 |
| | aaaaaaacaag | | KSFSQRANLRAHQRTHTGEKPYKCPECGKSFSQRANLRAHQRTHTGEKPYKCPECGKSFSQRANLRAHQ | |
| | | | RTHTGEKPYKCPECGKSFSQAGHLASHQRTHTHPRAPIPKPFQYKCPECGKSFSHRTTLTNHQRTHTGKK | |
| | | | TS | |
| + | atagagTTtga | 260 | LEPGEKPYKCPECGKSFSDSGNLRVHQRTHTGEKPYKCPECGKSFSQRANLRAHQRTHTGEKPYKCPECG | 742 |
| | aaaaaaaaaaa | | KSFSQRANLRAHQRTHTGEKPYKCPECGKSFSQRANLRAHQRTHTGEKPYKCPECGKSFSQAGHLASHQ | |
| | c | | RTHTPNPHRRTDPSHKPFQYKCPECGKSFSRSDNLVRHQRTHTGEKPYKCPECGKSFSQKSSLIAHQRTHT | |
| | | | GKKTS | |
| + | gagTTtgaaaa | 261 | LEPGEKPYKCPECGKSFSRKDNLKNHQRTHTGEKPYKCPECGKSFSDSGNLRVHQRTHTGEKPYKCPECG | 743 |
| | aaaaaaaacaa | | KSFSQRANLRAHQRTHTGEKPYKCPECGKSFSQRANLRAHQRTHTGEKPYKCPECGKSFSQRANLRAHQ | |
| | g | | RTHTGEKPYKCPECGKSFSQAGHLASHQRTHTPNPHRRTDPSHKPFQYKCPECGKSFSRSDNLVRHQRTH | |
| | | | TGKKTS | |
| + | agagtttgaaaa | 262 | LEPGEKPYKCPECGKSFSDSGNLRVHQRTHTGEKPYKCPECGKSFSQRANLRAHQRTHTGEKPYKCPECG | 744 |
| | aaaaaaaac | | KSFSQRANLRAHQRTHTGEKPYKCPECGKSFSQRANLRAHQRTHTGEKPYKCPECGKSFSQAGHLASHQ | |
| | | | RTHTGEKPYKCPECGKSFSTSGSLVRHQRTHTGEKPYKCPECGKSFSQLAHLRAHQRTHTGKKTS | |
| + | gtttgaaaaaaa | 263 | LEPGEKPYKCPECGKSFSRKDNLKNHQRTHTGEKPYKCPECGKSFSDSGNLRVHQRTHTGEKPYKCPECG | 745 |
| | aaaaacaag | | KSFSQRANLRAHQRTHTGEKPYKCPECGKSFSQRANLRAHQRTHTGEKPYKCPECGKSFSQRANLRAHQ | |
| | | | RTHTGEKPYKCPECGKSFSQAGHLASHQRTHTGEKPYKCPECGKSFSTSGSLVRHQRTHTGKKTS | |
| + | tgaaaaaaaaaa | 264 | LEPGEKPYKCPECGKSFSQRAHLERHQRTHTGEKPYKCPECGKSFSRKDNLKNHQRTHTGEKPYKCPECG | 746 |
| | aacaaggga | | KSFSDSGNLRVHQRTHTGEKPYKCPECGKSFSQRANLRAHQRTHTGEKPYKCPECGKSFSQRANLRAHQ | |
| | | | RTHTGEKPYKCPECGKSFSQRANLRAHQRTHTGEKPYKCPECGKSFSQAGHLASHQRTHTGKKTS | |
| + | aaaaaaaaaaa | 265 | LEPGEKPYKCPECGKSFSQRANLRAHQRTHTGEKPYKCPECGKSFSQRAHLERHQRTHTGEKPYKCPECG | 747 |
| | caagggaaaa | | KSFSRKDNLKNHQRTHTGEKPYKCPECGKSFSDSGNLRVHQRTHTGEKPYKCPECGKSFSQRANLRAHQ | |
| | | | RTHTGEKPYKCPECGKSFSQRANLRAHQRTHTGEKPYKCPECGKSFSQRANLRAHQRTHTGKKTS | |
| + | aaaaaaaacaa | 266 | LEPGEKPYKCPECGKSFSQSSNLVRHQRTHTGEKPYKCPECGKSFSQRANLRAHQRTHTGEKPYKCPECG | 748 |
| | gggaaaagaa | | KSFSQRAHLERHQRTHTGEKPYKCPECGKSFSRKDNLKNHQRTHTGEKPYKCPECGKSFSDSGNLRVHQ | |
| | | | RTHTGEKPYKCPECGKSFSQRANLRAHQRTHTGEKPYKCPECGKSFSQRANLRAHQRTHTGKKTS | |
| + | aaaaacaaggg | 267 | LEPGEKPYKCPECGKSFSQNSTLTEHQRTHTGEKPYKCPECGKSFSQSSNLVRHQRTHTGEKPYKCPECG | 749 |
| | aaaagaacta | | KSFSQRANLRAHQRTHTGEKPYKCPECGKSFSQRAHLERHQRTHTGEKPYKCPECGKSFSRKDNLKNHQ | |
| | | | RTHTGEKPYKCPECGKSFSDSGNLRVHQRTHTGEKPYKCPECGKSFSQRANLRAHQRTHTGKKTS | |
| + | aacaagggaaa | 268 | LEPGEKPYKCPECGKSFSQRANLRAHQRTHTGEKPYKCPECGKSFSQNSTLTEHQRTHTGEKPYKCPECG | 750 |
| | agaactaaaa | | KSFSQSSNLVRHQRTHTGEKPYKCPECGKSFSQRANLRAHQRTHTGEKPYKCPECGKSFSQRAHLERHQR | |
| | | | THTGEKPYKCPECGKSFSRKDNLKNHQRTHTGEKPYKCPECGKSFSDSGNLRVHQRTHTGKKTS | |
| + | aagggaaaaga | 269 | LEPGEKPYKCPECGKSFSQRANLRAHQRTHTHPRAPIPKPFQYKCPECGKSFSQRANLRAHQRTHTGEKP | 751 |
| | actaaaaTaaa | | YKCPECGKSFSQNSTLTEHQRTHTGEKPYKCPECGKSFSQSSNLVRHQRTHTGEKPYKCPECGKSFSQRA | |
| | | | NLRAHQRTHTGEKPYKCPECGKSFSQRAHLERHQRTHTGEKPYKCPECGKSFSRKDNLKNHQRTHTGKK | |
| | | | TS | |
| + | ggaaaagaact | 270 | LEPGEKPYKCPECGKSFSSKKALTEHQRTHTHPRAPIPKPFQYKCPECGKSFSQRANLRAHQRTHTHPRAP | 752 |
| | aaaaTaaaTca | | IPKPFQYKCPECGKSFSQRANLRAHQRTHTGEKPYKCPECGKSFSQNSTLTEHQRTHTGEKPYKCPECGKS | |
| | c | | FSQSSNLVRHQRTHTGEKPYKCPECGKSFSQRANLRAHQRTHTGEKPYKCPECGKSFSQRAHLERHQRTH | |
| | | | TGKKTS | |
| + | aaagaactaaaa | 271 | LEPGEKPYKCPECGKSFSRSDHLTNHQRTHTGEKPYKCPECGKSFSSKKALTEHQRTHTHPRAPIPKPFQY | 753 |
| | TaaaTcacag | | KCPECGKSFSQRANLRAHQRTHTHPRAPIPKPFQYKCPECGKSFSQRANLRAHQRTHTGEKPYKCPECGK | |
| | g | | SFSQNSTLTEHQRTHTGEKPYKCPECGKSFSQSSNLVRHQRTHTGEKPYKCPECGKSFSQRANLRAHQRT | |
| | | | HTGKKTS | |
| + | gaactaaaaTa | 272 | LEPGEKPYKCPECGKSFSDCRDLARHQRTHTGEKPYKCPECGKSFSRSDHLTNHQRTHTGEKPYKCPECG | 754 |
| | aaTcacagggc | | KSFSSKKALTEHQRTHTHPRAPIPKPFQYKCPECGKSFSQRANLRAHQRTHTHPRAPIPKPFQYKCPECGK | |
| | c | | SFSQRANLRAHQRTHTGEKPYKCPECGKSFSQNSTLTEHQRTHTGEKPYKCPECGKSFSQSSNLVRHQRT | |
| | | | HTGKKTS | |
| + | ctaaaaTaaaT | 273 | LEPGEKPYKCPECGKSFSDSGNLRVHQRTHTGEKPYKCPECGKSFSDCRDLARHQRTHTGEKPYKCPECG | 755 |
| | cacagggccaa | | KSFSRSDHLTNHQRTHTGEKPYKCPECGKSFSSKKALTEHQRTHTHPRAPIPKPFQYKCPECGKSFSQRAN | |
| | c | | LRAHQRTHTHPRAPIPKPFQYKCPECGKSFSQRANLRAHQRTHTGEKPYKCPECGKSFSQNSTLTEHQRT | |
| | | | HTGKKTS | |
| + | aaaTaaaTcac | 274 | LEPGEKPYKCPECGKSFSRNDTLTEHQRTHTGEKPYKCPECGKSFSDSGNLRVHQRTHTGEKPYKCPECG | 756 |
| | agggccaaccc | | KSFSDCRDLARHQRTHTGEKPYKCPECGKSFSRSDHLTNHQRTHTGEKPYKCPECGKSFSSKKALTEHQR | |
| | g | | THTHPRAPIPKPFQYKCPECGKSFSQRANLRAHQRTHTHPRAPIPKPFQYKCPECGKSFSQRANLRAHQRT | |
| | | | HTGKKTS | |
| + | gaaaaaaaaaa | 275 | LEPGEKPYKCPECGKSFSQSSNLVRHQRTHTGEKPYKCPECGKSFSRSDHLTNHQRTHTGEKPYKCPECG | 757 |
| | aacaagggaa | | KSFSSPADLTRHQRTHTGEKPYKCPECGKSFSQRANLRAHQRTHTGEKPYKCPECGKSFSQRANLRAHQR | |
| | | | THTGEKPYKCPECGKSFSQRANLRAHQRTHTGEKPYKCPECGKSFSQSSNLVRHQRTHTGKKTS | |
| + | aaaaaaaaaac | 276 | LEPGEKPYKCPECGKSFSRKDNLKNHQRTHTGEKPYKCPECGKSFSQSSNLVRHQRTHTGEKPYKCPECG | 758 |
| | aagggaaaag | | KSFSRSDHLTNHQRTHTGEKPYKCPECGKSFSSPADLTRHQRTHTGEKPYKCPECGKSFSQRANLRAHQR | |
| | | | THTGEKPYKCPECGKSFSQRANLRAHQRTHTGEKPYKCPECGKSFSQRANLRAHQRTHTGKKTS | |
| + | aaaaaaacaag | 277 | LEPGEKPYKCPECGKSFSDSGNLRVHQRTHTGEKPYKCPECGKSFSRKDNLKNHQRTHTGEKPYKCPECG | 759 |
| | ggaaaagaac | | KSFSQSSNLVRHQRTHTGEKPYKCPECGKSFSRSDHLTNHQRTHTGEKPYKCPECGKSFSSPADLTRHQR | |
| | | | THTGEKPYKCPECGKSFSQRANLRAHQRTHTGEKPYKCPECGKSFSQRANLRAHQRTHTGKKTS | |
| + | aaaacaaggga | 278 | LEPGEKPYKCPECGKSFSQRANLRAHQRTHTHPRAPIPKPFQYKCPECGKSFSDSGNLRVHQRTHTGEKP | 760 |
| | aaagaacTaaa | | YKCPECGKSFSRKDNLKNHQRTHTGEKPYKCPECGKSFSQSSNLVRHQRTHTGEKPYKCPECGKSFSRSD | |
| | | | HLTNHQRTHTGEKPYKCPECGKSFSSPADLTRHQRTHTGEKPYKCPECGKSFSQRANLRAHQRTHTGKK | |
| | | | TS | |
| + | acaagggaaaa | 279 | LEPGEKPYKCPECGKSFSQKSSLIAHQRTHTGEKPYKCPECGKSFSQRANLRAHQRTHTHPRAPIPKPFQY | 761 |
| | gaacTaaaata | | KCPECGKSFSDSGNLRVHQRTHTGEKPYKCPECGKSFSRKDNLKNHQRTHTGEKPYKCPECGKSFSQSSN | |
| | | | LVRHQRTHTGEKPYKCPECGKSFSRSDHLTNHQRTHTGEKPYKCPECGKSFSSPADLTRHQRTHTGKKTS | |
| + | agggaaaagaa | 280 | LEPGEKPYKCPECGKSFSTTGNLTVHQRTHTGEKPYKCPECGKSFSQKSSLIAHQRTHTGEKPYKCPECGK | 762 |
| | cTaaaataaat | | SFSQRANLRAHQRTHTHPRAPIPKPFQYKCPECGKSFSDSGNLRVHQRTHTGEKPYKCPECGKSFSRKDN | |
| | | | LKNHQRTHTGEKPYKCPECGKSFSQSSNLVRHQRTHTGEKPYKCPECGKSFSRSDHLTNHQRTHTGKKTS | |
| + | gaaaagaacTa | 281 | LEPGEKPYKCPECGKSFSSKKALTEHQRTHTGEKPYKCPECGKSFSTTGNLTVHQRTHTGEKPYKCPECG | 763 |
| | aaataaatcac | | KSFSQKSSLIAHQRTHTGEKPYKCPECGKSFSQRANLRAHQRTHTHPRAPIPKPFQYKCPECGKSFSDSGN | |
| | | | LRVHQRTHTGEKPYKCPECGKSFSRKDNLKNHQRTHTGEKPYKCPECGKSFSQSSNLVRHQRTHTGKKT | |
| | | | S | |
| + | aagaacTaaaa | 282 | LEPGEKPYKCPECGKSFSRSDHLTNHQRTHTGEKPYKCPECGKSFSSKKALTEHQRTHTGEKPYKCPECG | 764 |
| | taaatcacagg | | KSFSTTGNLTVHQRTHTGEKPYKCPECGKSFSQKSSLIAHQRTHTGEKPYKCPECGKSFSQRANLRAHQR | |
| | | | THTHPRAPIPKPFQYKCPECGKSFSDSGNLRVHQRTHTGEKPYKCPECGKSFSRKDNLKNHQRTHTGKKT | |
| | | | S | |
| + | aacTaaaataa | 283 | LEPGEKPYKCPECGKSFSDCRDLARHQRTHTGEKPYKCPECGKSFSRSDHLTNHQRTHTGEKPYKCPECG | 765 |
| | atcacagggcc | | KSFSSKKALTEHQRTHTGEKPYKCPECGKSFSTTGNLTVHQRTHTGEKPYKCPECGKSFSQKSSLIAHQRT | |
| | | | HTGEKPYKCPECGKSFSQRANLRAHQRTHTHPRAPIPKPFQYKCPECGKSFSDSGNLRVHQRTHTGKKTS | |
| + | aaaaaaaaaaa | 284 | LEPGEKPYKCPECGKSFSQRANLRAHQRTHTGEKPYKCPECGKSFSRSDKLVRHQRTHTGEKPYKCPECG | 766 |
| | acaagggaaa | | KSFSQSGNLTEHQRTHTGEKPYKCPECGKSFSQRANLRAHQRTHTGEKPYKCPECGKSFSQRANLRAHQ | |
| | | | RTHTGEKPYKCPECGKSFSQRANLRAHQRTHTGEKPYKCPECGKSFSQRANLRAHQRTHTGKKTS | |
| + | aaaaaaaaaca | 285 | LEPGEKPYKCPECGKSFSQLAHLRAHQRTHTGEKPYKCPECGKSFSQRANLRAHQRTHTGEKPYKCPECG | 767 |
| | agggaaaaga | | KSFSRSDKLVRHQRTHTGEKPYKCPECGKSFSQSGNLTEHQRTHTGEKPYKCPECGKSFSQRANLRAHQR | |
| | | | THTGEKPYKCPECGKSFSQRANLRAHQRTHTGEKPYKCPECGKSFSQRANLRAHQRTHTGKKTS | |
| + | aaaaaacaagg | 286 | LEPGEKPYKCPECGKSFSTHLDLIRHQRTHTGEKPYKCPECGKSFSQLAHLRAHQRTHTGEKPYKCPECG | 768 |
| | gaaaagaact | | KSFSQRANLRAHQRTHTGEKPYKCPECGKSFSRSDKLVRHQRTHTGEKPYKCPECGKSFSQSGNLTEHQR | |
| | | | THTGEKPYKCPECGKSFSQRANLRAHQRTHTGEKPYKCPECGKSFSQRANLRAHQRTHTGKKTS | |
| + | aaacaagggaa | 287 | LEPGEKPYKCPECGKSFSQRANLRAHQRTHTGEKPYKCPECGKSFSTHLDLIRHQRTHTGEKPYKCPECG | 769 |
| | aagaactaaa | | KSFSQLAHLRAHQRTHTGEKPYKCPECGKSFSQRANLRAHQRTHTGEKPYKCPECGKSFSRSDKLVRHQ | |
| | | | RTHTGEKPYKCPECGKSFSQSGNLTEHQRTHTGEKPYKCPECGKSFSQRANLRAHQRTHTGKKTS | |
| + | caagggaaaag | 288 | LEPGEKPYKCPECGKSFSQKSSLIAHQRTHTGEKPYKCPECGKSFSQRANLRAHQRTHTGEKPYKCPECG | 770 |
| | aactaaaata | | KSFSTHLDLIRHQRTHTGEKPYKCPECGKSFSQLAHLRAHQRTHTGEKPYKCPECGKSFSQRANLRAHQR | |
| | | | THTGEKPYKCPECGKSFSRSDKLVRHQRTHTGEKPYKCPECGKSFSQSGNLTEHQRTHTGKKTS | |
| + | gggaaaagaac | 289 | LEPGEKPYKCPECGKSFSTTGNLTVHQRTHTGEKPYKCPECGKSFSQKSSLIAHQRTHTGEKPYKCPECGK | 771 |
| | taaaataaat | | SFSQRANLRAHQRTHTGEKPYKCPECGKSFSTHLDLIRHQRTHTGEKPYKCPECGKSFSQLAHLRAHQRT | |
| | | | HTGEKPYKCPECGKSFSQRANLRAHQRTHTGEKPYKCPECGKSFSRSDKLVRHQRTHTGKKTS | |
| + | aaaagaactaaa | 290 | LEPGEKPYKCPECGKSFSSKKALTEHQRTHTGEKPYKCPECGKSFSTTGNLTVHQRTHTGEKPYKCPECG | 772 |
| | ataaatcac | | KSFSQKSSLIAHQRTHTGEKPYKCPECGKSFSQRANLRAHQRTHTGEKPYKCPECGKSFSTHLDLIRHQRT | |
| | | | HTGEKPYKCPECGKSFSQLAHLRAHQRTHTGEKPYKCPECGKSFSQRANLRAHQRTHTGKKTS | |
| + | agaactaaaata | 291 | LEPGEKPYKCPECGKSFSRSDHLTNHQRTHTGEKPYKCPECGKSFSSKKALTEHQRTHTGEKPYKCPECG | 773 |
| | aatcacagg | | KSFSTTGNLTVHQRTHTGEKPYKCPECGKSFSQKSSLIAHQRTHTGEKPYKCPECGKSFSQRANLRAHQR | |
| | | | THTGEKPYKCPECGKSFSTHLDLIRHQRTHTGEKPYKCPECGKSFSQLAHLRAHQRTHTGKKTS | |
| + | actaaaataaat | 292 | LEPGEKPYKCPECGKSFSDCRDLARHQRTHTGEKPYKCPECGKSFSRSDHLTNHQRTHTGEKPYKCPECG | 774 |
| | cacagggcc | | KSFSSKKALTEHQRTHTGEKPYKCPECGKSFSTTGNLTVHQRTHTGEKPYKCPECGKSFSQKSSLIAHQRT | |
| | | | HTGEKPYKCPECGKSFSQRANLRAHQRTHTGEKPYKCPECGKSFSTHLDLIRHQRTHTGKKTS | |
| + | aaaataaatcac | 293 | LEPGEKPYKCPECGKSFSDSGNLRVHQRTHTGEKPYKCPECGKSFSDCRDLARHQRTHTGEKPYKCPECG | 775 |
| | agggccaac | | KSFSRSDHLTNHQRTHTGEKPYKCPECGKSFSSKKALTEHQRTHTGEKPYKCPECGKSFSTTGNLTVHQR | |
| | | | THTGEKPYKCPECGKSFSQKSSLIAHQRTHTGEKPYKCPECGKSFSQRANLRAHQRTHTGKKTS | |
| + | ataaatcacagg | 294 | LEPGEKPYKCPECGKSFSRNDTLTEHQRTHTGEKPYKCPECGKSFSDSGNLRVHQRTHTGEKPYKCPECG | 776 |
| | gccaacccg | | KSFSDCRDLARHQRTHTGEKPYKCPECGKSFSRSDHLTNHQRTHTGEKPYKCPECGKSFSSKKALTEHQR | |
| | | | THTGEKPYKCPECGKSFSTTGNLTVHQRTHTGEKPYKCPECGKSFSQKSSLIAHQRTHTGKKTS | |
| + | aatcacagggc | 295 | LEPGEKPYKCPECGKSFSRSDHLTNHQRTHTGEKPYKCPECGKSFSRNDTLTEHQRTHTGEKPYKCPECG | 777 |
| | caacccgagg | | KSFSDSGNLRVHQRTHTGEKPYKCPECGKSFSDCRDLARHQRTHTGEKPYKCPECGKSFSRSDHLTNHQR | |
| | | | THTGEKPYKCPECGKSFSSKKALTEHQRTHTGEKPYKCPECGKSFSTTGNLTVHQRTHTGKKTS | |
| + | cacagggccaa | 296 | LEPGEKPYKCPECGKSFSRADNLTEHQRTHTGEKPYKCPECGKSFSRSDHLTNHQRTHTGEKPYKCPECG | 778 |
| | cccgaggcag | | KSFSRNDTLTEHQRTHTGEKPYKCPECGKSFSDSGNLRVHQRTHTGEKPYKCPECGKSFSDCRDLARHQR | |
| | | | THTGEKPYKCPECGKSFSRSDHLTNHQRTHTGEKPYKCPECGKSFSSKKALTEHQRTHTGKKTS | |
| + | agggccaaccc | 297 | LEPGEKPYKCPECGKSFSQSGDLRRHQRTHTGEKPYKCPECGKSFSRADNLTEHQRTHTGEKPYKCPECG | 779 |
| | gaggcaggca | | KSFSRSDHLTNHQRTHTGEKPYKCPECGKSFSRNDTLTEHQRTHTGEKPYKCPECGKSFSDSGNLRVHQR | |
| | | | THTGEKPYKCPECGKSFSDCRDLARHQRTHTGEKPYKCPECGKSFSRSDHLTNHQRTHTGKKTS | |
| + | gccaacccgag | 298 | LEPGEKPYKCPECGKSFSRSDNLVRHQRTHTGEKPYKCPECGKSFSQSGDLRRHQRTHTGEKPYKCPECG | 780 |
| | gcaggcagag | | KSFSRADNLTEHQRTHTGEKPYKCPECGKSFSRSDHLTNHQRTHTGEKPYKCPECGKSFSRNDTLTEHQR | |
| | | | THTGEKPYKCPECGKSFSDSGNLRVHQRTHTGEKPYKCPECGKSFSDCRDLARHQRTHTGKKTS | |
| + | aacccgaggca | 299 | LEPGEKPYKCPECGKSFSSPADLTRHQRTHTGEKPYKCPECGKSFSRSDNLVRHQRTHTGEKPYKCPECG | 781 |
| | ggcagagaca | | KSFSQSGDLRRHQRTHTGEKPYKCPECGKSFSRADNLTEHQRTHTGEKPYKCPECGKSFSRSDHLTNHQR | |
| | | | THTGEKPYKCPECGKSFSRNDTLTEHQRTHTGEKPYKCPECGKSFSDSGNLRVHQRTHTGKKTS | |
| + | ccgaggcaggc | 300 | LEPGEKPYKCPECGKSFSTSHSLTEHQRTHTGEKPYKCPECGKSFSSPADLTRHQRTHTGEKPYKCPECGK | 782 |
| | agagacacca | | SFSRSDNLVRHQRTHTGEKPYKCPECGKSFSQSGDLRRHQRTHTGEKPYKCPECGKSFSRADNLTEHQRT | |
| | | | HTGEKPYKCPECGKSFSRSDHLTNHQRTHTGEKPYKCPECGKSFSRNDTLTEHQRTHTGKKTS | |
| + | aggcaggcaga | 301 | LEPGEKPYKCPECGKSFSRNDALTEHQRTHTPNPHRRTDPSHKPFQYKCPECGKSFSTSHSLTEHQRTHTG | 783 |
| | gacaccaTTct | | EKPYKCPECGKSFSSPADLTRHQRTHTGEKPYKCPECGKSFSRSDNLVRHQRTHTGEKPYKCPECGKSFS | |
| | g | | QSGDLRRHQRTHTGEKPYKCPECGKSFSRADNLTEHQRTHTGEKPYKCPECGKSFSRSDHLTNHQRTHT | |
| | | | GKKTS | |
| + | caggcagagac | 302 | LEPGEKPYKCPECGKSFSQAGHLASHQRTHTGEKPYKCPECGKSFSRNDALTEHQRTHTPNPHRRTDPSH | 784 |
| | accaTTctgtg | | KPFQYKCPECGKSFSTSHSLTEHQRTHTGEKPYKCPECGKSFSSPADLTRHQRTHTGEKPYKCPECGKSFS | |
| | a | | RSDNLVRHQRTHTGEKPYKCPECGKSFSQSGDLRRHQRTHTGEKPYKCPECGKSFSRADNLTEHQRTHT | |
| | | | GKKTS | |
| + | gcagagacacc | 303 | LEPGEKPYKCPECGKSFSRSDELVRHQRTHTGEKPYKCPECGKSFSQAGHLASHQRTHTGEKPYKCPECG | 785 |
| | aTTctgtgagt | | KSFSRNDALTEHQRTHTPNPHRRTDPSHKPFQYKCPECGKSFSTSHSLTEHQRTHTGEKPYKCPECGKSFS | |
| | g | | SPADLTRHQRTHTGEKPYKCPECGKSFSRSDNLVRHQRTHTGEKPYKCPECGKSFSQSGDLRRHQRTHTG | |
| | | | KKTS | |
| + | gagacaccaT | 304 | LEPGEKPYKCPECGKSFSQLAHLRAHQRTHTGEKPYKCPECGKSFSRSDELVRHQRTHTGEKPYKCPECG | 786 |
| | Tctgtgagtga | | KSFSQAGHLASHQRTHTGEKPYKCPECGKSFSRNDALTEHQRTHTPNPHRRTDPSHKPFQYKCPECGKSF | |
| | ga | | STSHSLTEHQRTHTGEKPYKCPECGKSFSSPADLTRHQRTHTGEKPYKCPECGKSFSRSDNLVRHQRTHT | |
| | | | GKKTS | |
| + | acaccaTTctg | 305 | LEPGEKPYKCPECGKSFSQRAHLERHQRTHTGEKPYKCPECGKSFSQLAHLRAHQRTHTGEKPYKCPECG | 787 |
| | tgagtgagagg | | KSFSRSDELVRHQRTHTGEKPYKCPECGKSFSQAGHLASHQRTHTGEKPYKCPECGKSFSRNDALTEHQR | |
| | a | | THTPNPHRRTDPSHKPFQYKCPECGKSFSTSHSLTEHQRTHTGEKPYKCPECGKSFSSPADLTRHQRTHTG | |
| | | | KKTS | |
| + | ccaTTctgtga | 306 | LEPGEKPYKCPECGKSFSHKNALQNHQRTHTHPRAPIPKPFQYKCPECGKSFSQRAHLERHQRTHTGEKP | 788 |
| | gtgagaggaTa | | YKCPECGKSFSQLAHLRAHQRTHTGEKPYKCPECGKSFSRSDELVRHQRTHTGEKPYKCPECGKSFSQAG | |
| | tt | | HLASHQRTHTGEKPYKCPECGKSFSRNDALTEHQRTHTPNPHRRTDPSHKPFQYKCPECGKSFSTSHSLTE | |
| | | | HQRTHTGKKTS | |
| + | aataaaTcaca | 307 | LEPGEKPYKCPECGKSFSRNDTLTEHQRTHTGEKPYKCPECGKSFSDSGNLRVHQRTHTGEKPYKCPECG | 789 |
| | gggccaacccg | | KSFSDCRDLARHQRTHTGEKPYKCPECGKSFSRSDHLTNHQRTHTGEKPYKCPECGKSFSSKKALTEHQR | |
| | | | THTHPRAPIPKPFQYKCPECGKSFSQRANLRAHQRTHTGEKPYKCPECGKSFSTTGNLTVHQRTHTGKKT | |
| | | | S | |
| + | aaaTcacaggg | 308 | LEPGEKPYKCPECGKSFSRSDHLTNHQRTHTGEKPYKCPECGKSFSRNDTLTEHQRTHTGEKPYKCPECG | 790 |
| | ccaacccgagg | | KSFSDSGNLRVHQRTHTGEKPYKCPECGKSFSDCRDLARHQRTHTGEKPYKCPECGKSFSRSDHLTNHQR | |
| | | | THTGEKPYKCPECGKSFSSKKALTEHQRTHTHPRAPIPKPFQYKCPECGKSFSQRANLRAHQRTHTGKKT | |
| | | | S | |
| + | acagggccaac | 309 | LEPGEKPYKCPECGKSFSRSDHLTNHQRTHTGEKPYKCPECGKSFSDPGHLVRHQRTHTGEKPYKCPECG | 791 |
| | ccgaggcagg | | KSFSQSGHLTEHQRTHTGEKPYKCPECGKSFSDKKDLTRHQRTHTGEKPYKCPECGKSFSTSHSLTEHQR | |
| | | | THTGEKPYKCPECGKSFSRSDKLVRHQRTHTGEKPYKCPECGKSFSSPADLTRHQRTHTGKKTS | |
| + | gggccaacccg | 310 | LEPGEKPYKCPECGKSFSRADNLTEHQRTHTGEKPYKCPECGKSFSRSDHLTNHQRTHTGEKPYKCPECG | 792 |
| | aggcaggcag | | KSFSDPGHLVRHQRTHTGEKPYKCPECGKSFSQSGHLTEHQRTHTGEKPYKCPECGKSFSDKKDLTRHQR | |
| | | | THTGEKPYKCPECGKSFSTSHSLTEHQRTHTGEKPYKCPECGKSFSRSDKLVRHQRTHTGKKTS | |
| + | ccaacccgagg | 311 | LEPGEKPYKCPECGKSFSQLAHLRAHQRTHTGEKPYKCPECGKSFSRADNLTEHQRTHTGEKPYKCPECG | 793 |
| | caggcagaga | | KSFSRSDHLTNHQRTHTGEKPYKCPECGKSFSDPGHLVRHQRTHTGEKPYKCPECGKSFSQSGHLTEHQR | |
| | | | THTGEKPYKCPECGKSFSDKKDLTRHQRTHTGEKPYKCPECGKSFSTSHSLTEHQRTHTGKKTS | |
| + | acccgaggcag | 312 | LEPGEKPYKCPECGKSFSSKKALTEHQRTHTGEKPYKCPECGKSFSQLAHLRAHQRTHTGEKPYKCPECG | 794 |
| | gcagagacac | | KSFSRADNLTEHQRTHTGEKPYKCPECGKSFSRSDHLTNHQRTHTGEKPYKCPECGKSFSDPGHLVRHQR | |
| | | | THTGEKPYKCPECGKSFSQSGHLTEHQRTHTGEKPYKCPECGKSFSDKKDLTRHQRTHTGKKTS | |
| + | cgaggcaggca | 313 | LEPGEKPYKCPECGKSFSTSGNLTEHQRTHTGEKPYKCPECGKSFSSKKALTEHQRTHTGEKPYKCPECG | 795 |
| | gagacaccat | | KSFSQLAHLRAHQRTHTGEKPYKCPECGKSFSRADNLTEHQRTHTGEKPYKCPECGKSFSRSDHLTNHQR | |
| | | | THTGEKPYKCPECGKSFSDPGHLVRHQRTHTGEKPYKCPECGKSFSQSGHLTEHQRTHTGKKTS | |
| + | ggcaggcagag | 314 | LEPGEKPYKCPECGKSFSRNDALTEHQRTHTHPRAPIPKPFQYKCPECGKSFSTSGNLTEHQRTHTGEKPY | 796 |
| | acaccatTctg | | KCPECGKSFSSKKALTEHQRTHTGEKPYKCPECGKSFSQLAHLRAHQRTHTGEKPYKCPECGKSFSRADN | |
| | | | LTEHQRTHTGEKPYKCPECGKSFSRSDHLTNHQRTHTGEKPYKCPECGKSFSDPGHLVRHQRTHTGKKTS | |
| + | aggcagagaca | 315 | LEPGEKPYKCPECGKSFSQAGHLASHQRTHTGEKPYKCPECGKSFSRNDALTEHQRTHTHPRAPIPKPFQY | 797 |
| | ccatTctgtga | | KCPECGKSFSTSGNLTEHQRTHTGEKPYKCPECGKSFSSKKALTEHQRTHTGEKPYKCPECGKSFSQLAH | |
| | | | LRAHQRTHTGEKPYKCPECGKSFSRADNLTEHQRTHTGEKPYKCPECGKSFSRSDHLTNHQRTHTGKKTS | |
| + | cagagacacca | 316 | LEPGEKPYKCPECGKSFSRSDELVRHQRTHTGEKPYKCPECGKSFSQAGHLASHQRTHTGEKPYKCPECG | 798 |
| | tTctgtgagtg | | KSFSRNDALTEHQRTHTHPRAPIPKPFQYKCPECGKSFSTSGNLTEHQRTHTGEKPYKCPECGKSFSSKKA | |
| | | | LTEHQRTHTGEKPYKCPECGKSFSQLAHLRAHQRTHTGEKPYKCPECGKSFSRADNLTEHQRTHTGKKTS | |
| + | agacaccatTct | 317 | LEPGEKPYKCPECGKSFSQLAHLRAHQRTHTGEKPYKCPECGKSFSRSDELVRHQRTHTGEKPYKCPECG | 799 |
| | gtgagtgaga | | KSFSQAGHLASHQRTHTGEKPYKCPECGKSFSRNDALTEHQRTHTHPRAPIPKPFQYKCPECGKSFSTSGN | |
| | | | LTEHQRTHTGEKPYKCPECGKSFSSKKALTEHQRTHTGEKPYKCPECGKSFSQLAHLRAHQRTHTGKKTS | |
| + | caccatTctgtg | 318 | LEPGEKPYKCPECGKSFSQRAHLERHQRTHTGEKPYKCPECGKSFSQLAHLRAHQRTHTGEKPYKCPECG | 800 |
| | agtgagagga | | KSFSRSDELVRHQRTHTGEKPYKCPECGKSFSQAGHLASHQRTHTGEKPYKCPECGKSFSRNDALTEHQR | |
| | | | THTHPRAPIPKPFQYKCPECGKSFSTSGNLTEHQRTHTGEKPYKCPECGKSFSSKKALTEHQRTHTGKKTS | |
| + | catTctgtgagt | 319 | LEPGEKPYKCPECGKSFSHKNALQNHQRTHTHPRAPIPKPFQYKCPECGKSFSQRAHLERHQRTHTGEKP | 801 |
| | gagaggaTatt | | YKCPECGKSFSQLAHLRAHQRTHTGEKPYKCPECGKSFSRSDELVRHQRTHTGEKPYKCPECGKSFSQAG | |
| | | | HLASHQRTHTGEKPYKCPECGKSFSRNDALTEHQRTHTHPRAPIPKPFQYKCPECGKSFSTSGNLTEHQRT | |
| | | | HTGKKTS | |
| + | cagggccaacc | 320 | LEPGEKPYKCPECGKSFSDPGHLVRHQRTHTGEKPYKCPECGKSFSQSGDLRRHQRTHTGEKPYKCPECG | 802 |
| | cgaggcaggc | | KSFSRSDNLVRHQRTHTGEKPYKCPECGKSFSSKKHLAEHQRTHTGEKPYKCPECGKSFSQSGNLTEHQR | |
| | | | THTGEKPYKCPECGKSFSDPGHLVRHQRTHTGEKPYKCPECGKSFSRADNLTEHQRTHTGKKTS | |
| + | ggccaacccga | 321 | LEPGEKPYKCPECGKSFSQLAHLRAHQRTHTGEKPYKCPECGKSFSDPGHLVRHQRTHTGEKPYKCPECG | 803 |
| | ggcaggcaga | | KSFSQSGDLRRHQRTHTGEKPYKCPECGKSFSRSDNLVRHQRTHTGEKPYKCPECGKSFSSKKHLAEHQR | |
| | | | THTGEKPYKCPECGKSFSQSGNLTEHQRTHTGEKPYKCPECGKSFSDPGHLVRHQRTHTGKKTS | |
| + | caacccgaggc | 322 | LEPGEKPYKCPECGKSFSDPGNLVRHQRTHTGEKPYKCPECGKSFSQLAHLRAHQRTHTGEKPYKCPECG | 804 |
| | aggcagagac | | KSFSDPGHLVRHQRTHTGEKPYKCPECGKSFSQSGDLRRHQRTHTGEKPYKCPECGKSFSRSDNLVRHQR | |
| | | | THTGEKPYKCPECGKSFSSKKHLAEHQRTHTGEKPYKCPECGKSFSQSGNLTEHQRTHTGKKTS | |
| + | cccgaggcagg | 323 | LEPGEKPYKCPECGKSFSDKKDLTRHQRTHTGEKPYKCPECGKSFSDPGNLVRHQRTHTGEKPYKCPECG | 805 |
| | cagagacacc | | KSFSQLAHLRAHQRTHTGEKPYKCPECGKSFSDPGHLVRHQRTHTGEKPYKCPECGKSFSQSGDLRRHQR | |
| | | | THTGEKPYKCPECGKSFSRSDNLVRHQRTHTGEKPYKCPECGKSFSSKKHLAEHQRTHTGKKTS | |
| + | gaggcaggcag | 324 | LEPGEKPYKCPECGKSFSHKNALQNHQRTHTGEKPYKCPECGKSFSDKKDLTRHQRTHTGEKPYKCPEC | 806 |
| | agacaccatt | | GKSFSDPGNLVRHQRTHTGEKPYKCPECGKSFSQLAHLRAHQRTHTGEKPYKCPECGKSFSDPGHLVRH | |
| | | | QRTHTGEKPYKCPECGKSFSQSGDLRRHQRTHTGEKPYKCPECGKSFSRSDNLVRHQRTHTGKKTS | |
| + | gcaggcagaga | 325 | LEPGEKPYKCPECGKSFSRNDALTEHQRTHTGEKPYKCPECGKSFSHKNALQNHQRTHTGEKPYKCPECG | 807 |
| | caccattctg | | KSFSDKKDLTRHQRTHTGEKPYKCPECGKSFSDPGNLVRHQRTHTGEKPYKCPECGKSFSQLAHLRAHQ | |
| | | | RTHTGEKPYKCPECGKSFSDPGHLVRHQRTHTGEKPYKCPECGKSFSQSGDLRRHQRTHTGKKTS | |
| + | ggcagagacac | 326 | LEPGEKPYKCPECGKSFSQAGHLASHQRTHTGEKPYKCPECGKSFSRNDALTEHQRTHTGEKPYKCPECG | 808 |
| | cattctgtga | | KSFSHKNALQNHQRTHTGEKPYKCPECGKSFSDKKDLTRHQRTHTGEKPYKCPECGKSFSDPGNLVRHQ | |
| | | | RTHTGEKPYKCPECGKSFSQLAHLRAHQRTHTGEKPYKCPECGKSFSDPGHLVRHQRTHTGKKTS | |
| + | agagacaccatt | 327 | LEPGEKPYKCPECGKSFSRSDELVRHQRTHTGEKPYKCPECGKSFSQAGHLASHQRTHTGEKPYKCPECG | 809 |
| | ctgtgagtg | | KSFSRNDALTEHQRTHTGEKPYKCPECGKSFSHKNALQNHQRTHTGEKPYKCPECGKSFSDKKDLTRHQ | |
| | | | RTHTGEKPYKCPECGKSFSDPGNLVRHQRTHTGEKPYKCPECGKSFSQLAHLRAHQRTHTGKKTS | |
| + | gacaccattctg | 328 | LEPGEKPYKCPECGKSFSQLAHLRAHQRTHTGEKPYKCPECGKSFSRSDELVRHQRTHTGEKPYKCPECG | 810 |
| | tgagtgaga | | KSFSQAGHLASHQRTHTGEKPYKCPECGKSFSRNDALTEHQRTHTGEKPYKCPECGKSFSHKNALQNHQ | |
| | | | RTHTGEKPYKCPECGKSFSDKKDLTRHQRTHTGEKPYKCPECGKSFSDPGNLVRHQRTHTGKKTS | |
| + | accattctgtga | 329 | LEPGEKPYKCPECGKSFSQRAHLERHQRTHTGEKPYKCPECGKSFSQLAHLRAHQRTHTGEKPYKCPECG | 811 |
| | gtgagagga | | KSFSRSDELVRHQRTHTGEKPYKCPECGKSFSQAGHLASHQRTHTGEKPYKCPECGKSFSRNDALTEHQR | |
| | | | THTGEKPYKCPECGKSFSHKNALQNHQRTHTGEKPYKCPECGKSFSDKKDLTRHQRTHTGKKTS | |
| + | attctgtgagtga | 330 | LEPGEKPYKCPECGKSFSHKNALQNHQRTHTHPRAPIPKPFQYKCPECGKSFSQRAHLERHQRTHTGEKP | 812 |
| | gaggaTatt | | YKCPECGKSFSQLAHLRAHQRTHTGEKPYKCPECGKSFSRSDELVRHQRTHTGEKPYKCPECGKSFSQAG | |
| | | | HLASHQRTHTGEKPYKCPECGKSFSRNDALTEHQRTHTGEKPYKCPECGKSFSHKNALQNHQRTHTGKK | |
| | | | TS | |
| + | ctgtgagtgaga | 331 | LEPGEKPYKCPECGKSFSQAGHLASHQRTHTGEKPYKCPECGKSFSHKNALQNHQRTHTHPRAPIPKPFQ | 813 |
| | ggaTatttga | | YKCPECGKSFSQRAHLERHQRTHTGEKPYKCPECGKSFSQLAHLRAHQRTHTGEKPYKCPECGKSFSRSD | |
| | | | ELVRHQRTHTGEKPYKCPECGKSFSQAGHLASHQRTHTGEKPYKCPECGKSFSRNDALTEHQRTHTGKK | |
| | | | TS | |
| + | tgagtgagagg | 332 | LEPGEKPYKCPECGKSFSRSDKLVRHQRTHTGEKPYKCPECGKSFSQAGHLASHQRTHTGEKPYKCPECG | 814 |
| | aTatttgaggg | | KSFSHKNALQNHQRTHTHPRAPIPKPFQYKCPECGKSFSQRAHLERHQRTHTGEKPYKCPECGKSFSQLA | |
| | | | HLRAHQRTHTGEKPYKCPECGKSFSRSDELVRHQRTHTGEKPYKCPECGKSFSQAGHLASHQRTHTGKK | |
| | | | TS | |
| + | gtgagtgagag | 333 | LEPGEKPYKCPECGKSFSRSDKLVRHQRTHTGEKPYKCPECGKSFSQAGHLASHQRTHTPNPHRRTDPSH | 815 |
| | gataTTtgagg | | KPFQYKCPECGKSFSQKSSLIAHQRTHTGEKPYKCPECGKSFSRSDHLTNHQRTHTGEKPYKCPECGKSFS | |
| | g | | RSDNLVRHQRTHTGEKPYKCPECGKSFSHRTTLTNHQRTHTGEKPYKCPECGKSFSRSDELVRHQRTHTG | |
| | | | KKTS | |
| + | gagtgagagga | 334 | LEPGEKPYKCPECGKSFSRSDKLVRHQRTHTGEKPYKCPECGKSFSQAGHLASHQRTHTGEKPYKCPECG | 816 |
| | tatttgaggg | | KSFSHKNALQNHQRTHTGEKPYKCPECGKSFSTSGNLVRHQRTHTGEKPYKCPECGKSFSRSDNLVRHQ | |
| | | | RTHTGEKPYKCPECGKSFSQAGHLASHQRTHTGEKPYKCPECGKSFSRSDNLVRHQRTHTGKKTS | |
| + | gagggtCTct | 335 | LEPGEKPYKCPECGKSFSQLAHLRAHQRTHTGEKPYKCPECGKSFSQSSNLVRHQRTHTGEKPYKCPECG | 817 |
| | ggggaaagaaa | | KSFSQRANLRAHQRTHTGEKPYKCPECGKSFSRSDKLVRHQRTHTGEKPYKCPECGKSFSRNDALTEHQR | |
| | ga | | THTPNPHRRTDPSHKPFQYKCPECGKSFSTSGHLVRHQRTHTGEKPYKCPECGKSFSRSDNLVRHQRTHT | |
| | | | GKKTS | |
| + | ggtCTctggg | 336 | LEPGEKPYKCPECGKSFSQSSNLVRHQRTHTGEKPYKCPECGKSFSQLAHLRAHQRTHTGEKPYKCPECG | 818 |
| | gaaagaaagag | | KSFSQSSNLVRHQRTHTGEKPYKCPECGKSFSQRANLRAHQRTHTGEKPYKCPECGKSFSRSDKLVRHQR | |
| | aa | | THTGEKPYKCPECGKSFSRNDALTEHQRTHTPNPHRRTDPSHKPFQYKCPECGKSFSTSGHLVRHQRTHT | |
| | | | GKKTS | |
| + | agggtcTctgg | 337 | LEPGEKPYKCPECGKSFSQLAHLRAHQRTHTGEKPYKCPECGKSFSQSSNLVRHQRTHTGEKPYKCPECG | 819 |
| | ggaaagaaaga | | KSFSQRANLRAHQRTHTGEKPYKCPECGKSFSRSDKLVRHQRTHTGEKPYKCPECGKSFSRNDALTEHQR | |
| | | | THTHPRAPIPKPFQYKCPECGKSFSDPGALVRHQRTHTGEKPYKCPECGKSFSRSDHLTNHQRTHTGKKTS | |
| + | gtcTctgggga | 338 | LEPGEKPYKCPECGKSFSQSSNLVRHQRTHTGEKPYKCPECGKSFSQLAHLRAHQRTHTGEKPYKCPECG | 820 |
| | aagaaagagaa | | KSFSQSSNLVRHQRTHTGEKPYKCPECGKSFSQRANLRAHQRTHTGEKPYKCPECGKSFSRSDKLVRHQR | |
| | | | THTGEKPYKCPECGKSFSRNDALTEHQRTHTHPRAPIPKPFQYKCPECGKSFSDPGALVRHQRTHTGKKT | |
| | | | S | |
| + | ctggggaaaga | 339 | LEPGEKPYKCPECGKSFSRNDALTEHQRTHTHPRAPIPKPFQYKCPECGKSFSQSSNLVRHQRTHTGEKPY | 821 |
| | aagagaaTctg | | KCPECGKSFSQLAHLRAHQRTHTGEKPYKCPECGKSFSQSSNLVRHQRTHTGEKPYKCPECGKSFSQRAN | |
| | | | LRAHQRTHTGEKPYKCPECGKSFSRSDKLVRHQRTHTGEKPYKCPECGKSFSRNDALTEHQRTHTGKKT | |
| | | | S | |
| + | gggaaagaaag | 340 | LEPGEKPYKCPECGKSFSRKDNLKNHQRTHTGEKPYKCPECGKSFSRNDALTEHQRTHTHPRAPIPKPFQ | 822 |
| | agaaTctgaag | | YKCPECGKSFSQSSNLVRHQRTHTGEKPYKCPECGKSFSQLAHLRAHQRTHTGEKPYKCPECGKSFSQSS | |
| | | | NLVRHQRTHTGEKPYKCPECGKSFSQRANLRAHQRTHTGEKPYKCPECGKSFSRSDKLVRHQRTHTGKK | |
| | | | TS | |
| + | aaagaaagaga | 341 | LEPGEKPYKCPECGKSFSQNSTLTEHQRTHTPNPHRRTDPSHKPFQYKCPECGKSFSRKDNLKNHQRTHT | 823 |
| | aTctgaagCT | | GEKPYKCPECGKSFSRNDALTEHQRTHTHPRAPIPKPFQYKCPECGKSFSQSSNLVRHQRTHTGEKPYKCP | |
| | cta | | ECGKSFSQLAHLRAHQRTHTGEKPYKCPECGKSFSQSSNLVRHQRTHTGEKPYKCPECGKSFSQRANLRA | |
| | | | HQRTHTGKKTS | |
| + | gaaagagaaTc | 342 | LEPGEKPYKCPECGKSFSRSDELVRHQRTHTHPRAPIPKPFQYKCPECGKSFSQNSTLTEHQRTHTPNPHR | 824 |
| | tgaagCTctaT | | RTDPSHKPFQYKCPECGKSFSRKDNLKNHQRTHTGEKPYKCPECGKSFSRNDALTEHQRTHTHPRAPIPK | |
| | gtg | | PFQYKCPECGKSFSQSSNLVRHQRTHTGEKPYKCPECGKSFSQLAHLRAHQRTHTGEKPYKCPECGKSFS | |
| | | | QSSNLVRHQRTHTGKKTS | |
| + | agagaaTctga | 343 | LEPGEKPYKCPECGKSFSRSDHLTTHQRTHTGEKPYKCPECGKSFSRSDELVRHQRTHTHPRAPIPKPFQY | 825 |
| | agCTctaTgt | | KCPECGKSFSQNSTLTEHQRTHTPNPHRRTDPSHKPFQYKCPECGKSFSRKDNLKNHQRTHTGEKPYKCP | |
| | gtgg | | ECGKSFSRNDALTEHQRTHTHPRAPIPKPFQYKCPECGKSFSQSSNLVRHQRTHTGEKPYKCPECGKSFSQ | |
| | | | LAHLRAHQRTHTGKKTS | |
| + | gaaTctgaag | 344 | LEPGEKPYKCPECGKSFSRRDELNVHQRTHTGEKPYKCPECGKSFSRSDHLTTHQRTHTGEKPYKCPECG | 826 |
| | CTctaTgtgtg | | KSFSRSDELVRHQRTHTHPRAPIPKPFQYKCPECGKSFSQNSTLTEHQRTHTPNPHRRTDPSHKPFQYKCP | |
| | gatg | | ECGKSFSRKDNLKNHQRTHTGEKPYKCPECGKSFSRNDALTEHQRTHTHPRAPIPKPFQYKCPECGKSFSQS | |
| | | | SNLVRHQRTHTGKKTS | |
| + | tggggaaagaa | 345 | LEPGEKPYKCPECGKSFSRNDALTEHQRTHTGEKPYKCPECGKSFSTTGNLTVHQRTHTGEKPYKCPECG | 827 |
| | agagaatctg | | KSFSRSDNLVRHQRTHTGEKPYKCPECGKSFSQRANLRAHQRTHTGEKPYKCPECGKSFSRKDNLKNHQ | |
| | | | RTHTGEKPYKCPECGKSFSQRAHLERHQRTHTGEKPYKCPECGKSFSRSDHLTTHQRTHTGKKTS | |
| + | ggaaagaaaga | 346 | LEPGEKPYKCPECGKSFSRKDNLKNHQRTHTGEKPYKCPECGKSFSRNDALTEHQRTHTGEKPYKCPECG | 828 |
| | gaatctgaag | | KSFSTTGNLTVHQRTHTGEKPYKCPECGKSFSRSDNLVRHQRTHTGEKPYKCPECGKSFSQRANLRAHQR | |
| | | | THTGEKPYKCPECGKSFSRKDNLKNHQRTHTGEKPYKCPECGKSFSQRAHLERHQRTHTGKKTS | |
| + | aagaaagagaa | 347 | LEPGEKPYKCPECGKSFSQNSTLTEHQRTHTPNPHRRTDPSHKPFQYKCPECGKSFSRKDNLKNHQRTHT | 829 |
| | tctgaagCTct | | GEKPYKCPECGKSFSRNDALTEHQRTHTGEKPYKCPECGKSFSTTGNLTVHQRTHTGEKPYKCPECGKSF | |
| | a | | SRSDNLVRHQRTHTGEKPYKCPECGKSFSQRANLRAHQRTHTGEKPYKCPECGKSFSRKDNLKNHQRTH | |
| | | | TGKKTS | |
| + | aaagagaatctg | 348 | LEPGEKPYKCPECGKSFSRSDELVRHQRTHTHPRAPIPKPFQYKCPECGKSFSQNSTLTEHQRTHTPNPHR | 830 |
| | aagCTctaTgt | | RTDPSHKPFQYKCPECGKSFSRKDNLKNHQRTHTGEKPYKCPECGKSFSRNDALTEHQRTHTGEKPYKCP | |
| | g | | ECGKSFSTTGNLTVHQRTHTGEKPYKCPECGKSFSRSDNLVRHQRTHTGEKPYKCPECGKSFSQRANLRA | |
| | | | HQRTHTGKKTS | |
| + | gagaatctgaag | 349 | LEPGEKPYKCPECGKSFSRSDHLTTHQRTHTGEKPYKCPECGKSFSRSDELVRHQRTHTHPRAPIPKPFQY | 831 |
| | CTctaTgtgtg | | KCPECGKSFSQNSTLTEHQRTHTPNPHRRTDPSHKPFQYKCPECGKSFSRKDNLKNHQRTHTGEKPYKCP | |
| | g | | ECGKSFSRNDALTEHQRTHTGEKPYKCPECGKSFSTTGNLTVHQRTHTGEKPYKCPECGKSFSRSDNLVR | |
| | | | HQRTHTGKKTS | |
| + | aatctgaagCT | 350 | LEPGEKPYKCPECGKSFSRRDELNVHQRTHTGEKPYKCPECGKSFSRSDHLTTHQRTHTGEKPYKCPECG | 832 |
| | ctaTgtgtggat | | KSFSRSDELVRHQRTHTHPRAPIPKPFQYKCPECGKSFSQNSTLTEHQRTHTPNPHRRTDPSHKPFQYKCP | |
| | g | | ECGKSFSRKDNLKNHQRTHTGEKPYKCPECGKSFSRNDALTEHQRTHTGEKPYKCPECGKSFSTTGNLTVH | |
| | | | QRTHTGKKTS | |
| + | ctgaagCTcta | 351 | LEPGEKPYKCPECGKSFSQRAHLERHQRTHTGEKPYKCPECGKSFSRRDELNVHQRTHTGEKPYKCPECG | 833 |
| | Tgtgtggatgg | | KSFSRSDHLTTHQRTHTGEKPYKCPECGKSFSRSDELVRHQRTHTHPRAPIPKPFQYKCPECGKSFSQNST | |
| | ga | | LTEHQRTHTPNPHRRTDPSHKPFQYKCPECGKSFSRKDNLKNHQRTHTGEKPYKCPECGKSFSRNDALTE | |
| | | | HQRTHTGKKTS | |
| + | aagCTctaTgt | 352 | LEPGEKPYKCPECGKSFSTTGNLTVHQRTHTGEKPYKCPECGKSFSQRAHLERHQRTHTGEKPYKCPECG | 834 |
| | gtggatgggaa | | KSFSRRDELNVHQRTHTGEKPYKCPECGKSFSRSDHLTTHQRTHTGEKPYKCPECGKSFSRSDELVRHQR | |
| | at | | THTHPRAPIPKPFQYKCPECGKSFSQNSTLTEHQRTHTPNPHRRTDPSHKPFQYKCPECGKSFSRKDNLKN | |
| | | | HQRTHTGKKTS | |
| + | ggggaaagaaa | 353 | LEPGEKPYKCPECGKSFSRKDNLKNHQRTHTGEKPYKCPECGKSFSRNDALTEHQRTHTPNPHRRTDPSH | 835 |
| | gagaATctga | | KPFQYKCPECGKSFSQLAHLRAHQRTHTGEKPYKCPECGKSFSRKDNLKNHQRTHTGEKPYKCPECGKS | |
| | ag | | FSQLAHLRAHQRTHTGEKPYKCPECGKSFSQSSNLVRHQRTHTGEKPYKCPECGKSFSRSDKLVRHQRTH | |
| | | | TGKKTS | |
| + | gaaagaaagag | 354 | LEPGEKPYKCPECGKSFSQNSTLTEHQRTHTPNPHRRTDPSHKPFQYKCPECGKSFSRKDNLKNHQRTHT | 836 |
| | aATctgaagC | | GEKPYKCPECGKSFSRNDALTEHQRTHTPNPHRRTDPSHKPFQYKCPECGKSFSQLAHLRAHQRTHTGEK | |
| | Tcta | | PYKCPECGKSFSRKDNLKNHQRTHTGEKPYKCPECGKSFSQLAHLRAHQRTHTGEKPYKCPECGKSFSQS | |
| | | | SNLVRHQRTHTGKKTS | |
| + | agaaagagaA | 355 | LEPGEKPYKCPECGKSFSRSDELVRHQRTHTHPRAPIPKPFQYKCPECGKSFSQNSTLTEHQRTHTPNPHR | 837 |
| | TctgaagCTct | | RTDPSHKPFQYKCPECGKSFSRKDNLKNHQRTHTGEKPYKCPECGKSFSRNDALTEHQRTHTPNPHRRTD | |
| | aTgtg | | PSHKPFQYKCPECGKSFSQLAHLRAHQRTHTGEKPYKCPECGKSFSRKDNLKNHQRTHTGEKPYKCPEC | |
| | | | GKSFSQLAHLRAHQRTHTGKKTS | |
| + | aagagaATct | 356 | LEPGEKPYKCPECGKSFSRSDHLTTHQRTHTGEKPYKCPECGKSFSRSDELVRHQRTHTHPRAPIPKPFQY | 838 |
| | gaagCTctaT | | KCPECGKSFSQNSTLTEHQRTHTPNPHRRTDPSHKPFQYKCPECGKSFSRKDNLKNHQRTHTGEKPYKCP | |
| | gtgtgg | | ECGKSFSRNDALTEHQRTHTPNPHRRTDPSHKPFQYKCPECGKSFSQLAHLRAHQRTHTGEKPYKCPECG | |
| | | | KSFSRKDNLKNHQRTHTGKKTS | |
| + | agaATctgaa | 357 | LEPGEKPYKCPECGKSFSRRDELNVHQRTHTGEKPYKCPECGKSFSRSDHLTTHQRTHTGEKPYKCPECG | 839 |
| | gCTctaTgtgt | | KSFSRSDELVRHQRTHTHPRAPIPKPFQYKCPECGKSFSQNSTLTEHQRTHTPNPHRRTDPSHKPFQYKCP | |
| | ggatg | | ECGKSFSRKDNLKNHQRTHTGEKPYKCPECGKSFSRNDALTEHQRTHTPNPHRRTDPSHKPFQYKCPECGK | |
| | | | SFSQLAHLRAHQRTHTGKKTS | |
| + | tgaagcTctaT | 358 | LEPGEKPYKCPECGKSFSQRAHLERHQRTHTGEKPYKCPECGKSFSRRDELNVHQRTHTGEKPYKCPECG | 840 |
| | gtgtggatggg | | KSFSRSDHLTTHQRTHTGEKPYKCPECGKSFSRSDELVRHQRTHTHPRAPIPKPFQYKCPECGKSFSQNST | |
| | a | | LTEHQRTHTHPRAPIPKPFQYKCPECGKSFSERSHLREHQRTHTGEKPYKCPECGKSFSQAGHLASHQRTH | |
| | | | TGKKTS | |
| + | agcTctaTgtg | 359 | LEPGEKPYKCPECGKSFSTTGNLTVHQRTHTGEKPYKCPECGKSFSQRAHLERHQRTHTGEKPYKCPECG | 841 |
| | tggatgggaaat | | KSFSRRDELNVHQRTHTGEKPYKCPECGKSFSRSDHLTTHQRTHTGEKPYKCPECGKSFSRSDELVRHQR | |
| | | | THTHPRAPIPKPFQYKCPECGKSFSQNSTLTEHQRTHTHPRAPIPKPFQYKCPECGKSFSERSHLREHQRT | |
| | | | HTGKKTS | |
| + | gaagctctaTgt | 360 | LEPGEKPYKCPECGKSFSQRAHLERHQRTHTGEKPYKCPECGKSFSRRDELNVHQRTHTGEKPYKCPECG | 842 |
| | gtggatggga | | KSFSRSDHLTTHQRTHTGEKPYKCPECGKSFSRSDELVRHQRTHTHPRAPIPKPFQYKCPECGKSFSQNST | |
| | | | LTEHQRTHTGEKPYKCPECGKSFSTSGELVRHQRTHTGEKPYKCPECGKSFSQSSNLVRHQRTHTGKKTS | |
| + | gctctaTgtgtg | 361 | LEPGEKPYKCPECGKSFSTTGNLTVHQRTHTGEKPYKCPECGKSFSQRAHLERHQRTHTGEKPYKCPECG | 843 |
| | gatgggaaat | | KSFSRRDELNVHQRTHTGEKPYKCPECGKSFSRSDHLTTHQRTHTGEKPYKCPECGKSFSRSDELVRHQR | |
| | | | THTHPRAPIPKPFQYKCPECGKSFSQNSTLTEHQRTHTGEKPYKCPECGKSFSTSGELVRHQRTHTGKKTS | |
| + | ctaTgtgtggat | 362 | LEPGEKPYKCPECGKSFSDCRDLARHQRTHTGEKPYKCPECGKSFSTTGNLTVHQRTHTGEKPYKCPECG | 844 |
| | gggaaatgcc | | KSFSQRAHLERHQRTHTGEKPYKCPECGKSFSRRDELNVHQRTHTGEKPYKCPECGKSFSRSDHLTTHQR | |
| | | | THTGEKPYKCPECGKSFSRSDELVRHQRTHTHPRAPIPKPFQYKCPECGKSFSQNSTLTEHQRTHTGKKTS | |
| + | atgTgtggatg | 363 | LEPGEKPYKCPECGKSFSRSDHLTNHQRTHTGEKPYKCPECGKSFSDCRDLARHQRTHTHPRAPIPKPFQY | 845 |
| | ggaaaTgcca | | KCPECGKSFSQRANLRAHQRTHTGEKPYKCPECGKSFSRSDKLVRHQRTHTGEKPYKCPECGKSFSTSGN | |
| | gg | | LVRHQRTHTGEKPYKCPECGKSFSRSDELVRHQRTHTHPRAPIPKPFQYKCPECGKSFSRRDELNVHQRT | |
| | | | HTGKKTS | |
| - | attTcccATcc | 364 | LEPGEKPYKCPECGKSFSTTGALTEHQRTHTGEKPYKCPECGKSFSRSDNLVRHQRTHTGEKPYKCPECG | 846 |
| | acacatagagct | | KSFSQKSSLIAHQRTHTGEKPYKCPECGKSFSSKKALTEHQRTHTGEKPYKCPECGKSFSTSHSLTEHQRT | |
| | t | | HTPNPHRRTDPSHKPFQYKCPECGKSFSSKKHLAEHQRTHTHPRAPIPKPFQYKCPECGKSFSHKNALQNH | |
| | | | QRTHTGKKTS | |
| - | cccATccaca | 365 | LEPGEKPYKCPECGKSFSRADNLTEHQRTHTGEKPYKCPECGKSFSTTGALTEHQRTHTGEKPYKCPECG | 847 |
| | catagagcttca | | KSFSRSDNLVRHQRTHTGEKPYKCPECGKSFSQKSSLIAHQRTHTGEKPYKCPECGKSFSSKKALTEHQRT | |
| | g | | HTGEKPYKCPECGKSFSTSHSLTEHQRTHTPNPHRRTDPSHKPFQYKCPECGKSFSSKKHLAEHQRTHTG | |
| | | | KKTS | |
| - | ccaTccacaca | 366 | LEPGEKPYKCPECGKSFSRADNLTEHQRTHTGEKPYKCPECGKSFSTTGALTEHQRTHTGEKPYKCPECG | 848 |
| | tagagcttcag | | KSFSRSDNLVRHQRTHTGEKPYKCPECGKSFSQKSSLIAHQRTHTGEKPYKCPECGKSFSSKKALTEHQRT | |
| | | | HTGEKPYKCPECGKSFSTSHSLTEHQRTHTHPRAPIPKPFQYKCPECGKSFSTSHSLTEHQRTHTGKKTS | |
| - | catccacacata | 367 | LEPGEKPYKCPECGKSFSRADNLTEHQRTHTGEKPYKCPECGKSFSTTGALTEHQRTHTGEKPYKCPECG | 849 |
| | gagcttcag | | KSFSRSDNLVRHQRTHTGEKPYKCPECGKSFSQKSSLIAHQRTHTGEKPYKCPECGKSFSSKKALTEHQRT | |
| - | ccacacataga | 368 | LEPGEKPYKCPECGKSFSHKNALQNHQRTHTGEKPYKCPECGKSFSRADNLTEHQRTHTGEKPYKCPECG | 850 |
| | gcttcagatt | | KSFSTTGALTEHQRTHTGEKPYKCPECGKSFSRSDNLVRHQRTHTGEKPYKCPECGKSFSQKSSLIAHQRT | |
| | | | HTGEKPYKCPECGKSFSSKKALTEHQRTHTGEKPYKCPECGKSFSTSHSLTEHQRTHTGKKTS | |
| - | cacatagagctt | 369 | LEPGEKPYKCPECGKSFSTTGALTEHQRTHTPNPHRRTDPSHKPFQYKCPECGKSFSHKNALQNHQRTHT | 851 |
| | cagattCTctt | | GEKPYKCPECGKSFSRADNLTEHQRTHTGEKPYKCPECGKSFSTTGALTEHQRTHTGEKPYKCPECGKSF | |
| | | | SRSDNLVRHQRTHTGEKPYKCPECGKSFSQKSSLIAHQRTHTGEKPYKCPECGKSFSSKKALTEHQRTHT | |
| | | | GKKTS | |
| - | atagagcttcag | 370 | LEPGEKPYKCPECGKSFSTTGALTEHQRTHTHPRAPIPKPFQYKCPECGKSFSTTGALTEHQRTHTPNPHR | 852 |
| | attCTcttTctt | | RTDPSHKPFQYKCPECGKSFSHKNALQNHQRTHTGEKPYKCPECGKSFSRADNLTEHQRTHTGEKPYKCP | |
| | | | ECGKSFSTTGALTEHQRTHTGEKPYKCPECGKSFSRSDNLVRHQRTHTGEKPYKCPECGKSFSQKSSLIAH | |
| | | | QRTHTGKKTS | |
| - | gagcttcagatt | 371 | LEPGEKPYKCPECGKSFSSKKHLAEHQRTHTHPRAPIPKPFQYKCPECGKSFSTTGALTEHQRTHTHPRAP | 853 |
| | CTcttTcttTc | | IPKPFQYKCPECGKSFSTTGALTEHQRTHTPNPHRRTDPSHKPFQYKCPECGKSFSHKNALQNHQRTHTGE | |
| | CC | | KPYKCPECGKSFSRADNLTEHQRTHTGEKPYKCPECGKSFSTTGALTEHQRTHTGEKPYKCPECGKSFSR | |
| | | | SDNLVRHQRTHTGKKTS | |
| - | cttcagattCTc | 372 | LEPGEKPYKCPECGKSFSRADNLTEHQRTHTGEKPYKCPECGKSFSSKKHLAEHQRTHTHPRAPIPKPFQY | 854 |
| | ttTcttTcccca | | KCPECGKSFSTTGALTEHQRTHTHPRAPIPKPFQYKCPECGKSFSTTGALTEHQRTHTPNPHRRTDPSHKPF | |
| | g | | QYKCPECGKSFSHKNALQNHQRTHTGEKPYKCPECGKSFSRADNLTEHQRTHTGEKPYKCPECGKSFSTT | |
| | | | GALTEHQRTHTGKKTS | |
| - | cagattCTctt | 373 | LEPGEKPYKCPECGKSFSQLAHLRAHQRTHTGEKPYKCPECGKSFSRADNLTEHQRTHTGEKPYKCPECG | 855 |
| | TcttTccccag | | KSFSSKKHLAEHQRTHTHPRAPIPKPFQYKCPECGKSFSTTGALTEHQRTHTHPRAPIPKPFQYKCPECGK | |
| | aga | | SFSTTGALTEHQRTHTPNPHRRTDPSHKPFQYKCPECGKSFSHKNALQNHQRTHTGEKPYKCPECGKSFSR | |
| | | | ADNLTEHQRTHTGKKTS | |
| - | attCTcttTctt | 374 | LEPGEKPYKCPECGKSFSSKKHLAEHQRTHTGEKPYKCPECGKSFSQLAHLRAHQRTHTGEKPYKCPECG | 856 |
| | Tccccagagac | | KSFSRADNLTEHQRTHTGEKPYKCPECGKSFSSKKHLAEHQRTHTHPRAPIPKPFQYKCPECGKSFSTTGA | |
| | cc | | LTEHQRTHTHPRAPIPKPFQYKCPECGKSFSTTGALTEHQRTHTPNPHRRTDPSHKPFQYKCPECGKSFSH | |
| | | | KNALQNHQRTHTGKKTS | |
| - | cacacatagag | 375 | LEPGEKPYKCPECGKSFSTTGALTEHQRTHTPNPHRRTDPSHKPFQYKCPECGKSFSHKNALQNHQRTHT | 857 |
| | cTTcagattC | | GEKPYKCPECGKSFSRADNLTEHQRTHTPNPHRRTDPSHKPFQYKCPECGKSFSERSHLREHQRTHTGEK | |
| | Tctt | | PYKCPECGKSFSREDNLHTHQRTHTGEKPYKCPECGKSFSSPADLTRHQRTHTGEKPYKCPECGKSFSSK | |
| | | | KALTEHQRTHTGKKTS | |
| - | acatagagcTT | 376 | LEPGEKPYKCPECGKSFSTTGALTEHQRTHTHPRAPIPKPFQYKCPECGKSFSTTGALTEHQRTHTPNPHR | 858 |
| | cagattCTctt | | RTDPSHKPFQYKCPECGKSFSHKNALQNHQRTHTGEKPYKCPECGKSFSRADNLTEHQRTHTPNPHRRTD | |
| | Tctt | | PSHKPFQYKCPECGKSFSERSHLREHQRTHTGEKPYKCPECGKSFSREDNLHTHQRTHTGEKPYKCPECG | |
| | | | KSFSSPADLTRHQRTHTGKKTS | |
| - | tagagcTTcag | 377 | LEPGEKPYKCPECGKSFSSKKHLAEHQRTHTHPRAPIPKPFQYKCPECGKSFSTTGALTEHQRTHTHPRAP | 859 |
| | attCTcttTctt | | IPKPFQYKCPECGKSFSTTGALTEHQRTHTPNPHRRTDPSHKPFQYKCPECGKSFSHKNALQNHQRTHTGE | |
| | Tccc | | KPYKCPECGKSFSRADNLTEHQRTHTPNPHRRTDPSHKPFQYKCPECGKSFSERSHLREHQRTHTGEKPY | |
| | | | KCPECGKSFSREDNLHTHQRTHTGKKTS | |
| - | agcTTcagatt | 378 | LEPGEKPYKCPECGKSFSRADNLTEHQRTHTGEKPYKCPECGKSFSSKKHLAEHQRTHTHPRAPIPKPFQY | 860 |
| | CTcttTcttTc | | KCPECGKSFSTTGALTEHQRTHTHPRAPIPKPFQYKCPECGKSFSTTGALTEHQRTHTPNPHRRTDPSHKP | |
| | cccag | | FQYKCPECGKSFSHKNALQNHQRTHTGEKPYKCPECGKSFSRADNLTEHQRTHTPNPHRRTDPSHKPFQY | |
| | | | KCPECGKSFSERSHLREHQRTHTGKKTS | |
| - | acacatagagct | 379 | LEPGEKPYKCPECGKSFSTTGALTEHQRTHTPNPHRRTDPSHKPFQYKCPECGKSFSHKNALQNHQRTHT | 861 |
| | TcagattCTct | | GEKPYKCPECGKSFSRADNLTEHQRTHTHPRAPIPKPFQYKCPECGKSFSTSGELVRHQRTHTGEKPYKCP | |
| | t | | ECGKSFSQLAHLRAHQRTHTGEKPYKCPECGKSFSTSGNLTEHQRTHTGEKPYKCPECGKSFSSPADLTR | |
| | | | HQRTHTGKKTS | |
| - | catagagctTc | 380 | LEPGEKPYKCPECGKSFSTTGALTEHQRTHTHPRAPIPKPFQYKCPECGKSFSTTGALTEHQRTHTPNPHR | 862 |
| | agattCTcttT | | RTDPSHKPFQYKCPECGKSFSHKNALQNHQRTHTGEKPYKCPECGKSFSRADNLTEHQRTHTHPRAPIPK | |
| | ctt | | PFQYKCPECGKSFSTSGELVRHQRTHTGEKPYKCPECGKSFSQLAHLRAHQRTHTGEKPYKCPECGKSFS | |
| | | | TSGNLTEHQRTHTGKKTS | |
| - | agagctTcaga | 381 | LEPGEKPYKCPECGKSFSSKKHLAEHQRTHTHPRAPIPKPFQYKCPECGKSFSTTGALTEHQRTHTHPRAP | 863 |
| | ttCTcttTcttT | | IPKPFQYKCPECGKSFSTTGALTEHQRTHTPNPHRRTDPSHKPFQYKCPECGKSFSHKNALQNHQRTHTGE | |
| | CCC | | KPYKCPECGKSFSRADNLTEHQRTHTHPRAPIPKPFQYKCPECGKSFSTSGELVRHQRTHTGEKPYKCPEC | |
| | | | GKSFSQLAHLRAHQRTHTGKKTS | |
| - | gctTcagattC | 382 | LEPGEKPYKCPECGKSFSRADNLTEHQRTHTGEKPYKCPECGKSFSSKKHLAEHQRTHTHPRAPIPKPFQY | 864 |
| | TcttTcttTccc | | KCPECGKSFSTTGALTEHQRTHTHPRAPIPKPFQYKCPECGKSFSTTGALTEHQRTHTPNPHRRTDPSHKP | |
| | cag | | FQYKCPECGKSFSHKNALQNHQRTHTGEKPYKCPECGKSFSRADNLTEHQRTHTHPRAPIPKPFQYKCPEC | |
| | | | GKSFSTSGELVRHQRTHTGKKTS | |
| - | cttTcttTcccc | 383 | LEPGEKPYKCPECGKSFSQSGNLTEHQRTHTHPRAPIPKPFQYKCPECGKSFSSKKHLAEHQRTHTGEKPY | 865 |
| | agagacccTca | | KCPECGKSFSQLAHLRAHQRTHTGEKPYKCPECGKSFSRADNLTEHQRTHTGEKPYKCPECGKSFSSKKH | |
| | a | | LAEHQRTHTHPRAPIPKPFQYKCPECGKSFSTTGALTEHQRTHTHPRAPIPKPFQYKCPECGKSFSTTGAL | |
| | | | TEHQRTHTGKKTS | |
| - | cttTccccaga | 384 | LEPGEKPYKCPECGKSFSQKSSLIAHQRTHTGEKPYKCPECGKSFSQSGNLTEHQRTHTHPRAPIPKPFQY | 866 |
| | gacccTcaaat | | KCPECGKSFSSKKHLAEHQRTHTGEKPYKCPECGKSFSQLAHLRAHQRTHTGEKPYKCPECGKSFSRADNL | |
| | a | | TEHQRTHTGEKPYKCPECGKSFSSKKHLAEHQRTHTHPRAPIPKPFQYKCPECGKSFSTTGALTEHQRTHT | |
| | | | GKKTS | |
| - | ccccagagacc | 385 | LEPGEKPYKCPECGKSFSTKNSLTEHQRTHTHPRAPIPKPFQYKCPECGKSFSQKSSLIAHQRTHTGEKPY | 867 |
| | cTcaaataTcc | | KCPECGKSFSQSGNLTEHQRTHTHPRAPIPKPFQYKCPECGKSFSSKKHLAEHQRTHTGEKPYKCPECGKS | |
| | t | | FSQLAHLRAHQRTHTGEKPYKCPECGKSFSRADNLTEHQRTHTGEKPYKCPECGKSFSSKKHLAEHQRTH | |
| | | | TGKKTS | |
| - | cagagacccTc | 386 | LEPGEKPYKCPECGKSFSSKKALTEHQRTHTPNPHRRTDPSHKPFQYKCPECGKSFSTKNSLTEHQRTHTH | 868 |
| | aaataTcctCT | | PRAPIPKPFQYKCPECGKSFSQKSSLIAHQRTHTGEKPYKCPECGKSFSQSGNLTEHQRTHTHPRAPIPKP | |
| | cac | | FQYKCPECGKSFSSKKHLAEHQRTHTGEKPYKCPECGKSFSQLAHLRAHQRTHTGEKPYKCPECGKSFSRA | |
| | | | DNLTEHQRTHTGKKTS | |
| - | agacccTcaaa | 387 | LEPGEKPYKCPECGKSFSSKKALTEHQRTHTHPRAPIPKPFQYKCPECGKSFSSKKALTEHQRTHTPNPHR | 869 |
| | taTcctCTcac | | RTDPSHKPFQYKCPECGKSFSTKNSLTEHQRTHTHPRAPIPKPFQYKCPECGKSFSQKSSLIAHQRTHTGE | |
| | Tcac | | KPYKCPECGKSFSQSGNLTEHQRTHTHPRAPIPKPFQYKCPECGKSFSSKKHLAEHQRTHTGEKPYKCPECG | |
| | | | KSFSQLAHLRAHQRTHTGKKTS | |
| - | cccTcaaataT | 388 | LEPGEKPYKCPECGKSFSQLAHLRAHQRTHTGEKPYKCPECGKSFSSKKALTEHQRTHTHPRAPIPKPFQY | 870 |
| | cctCTcacTc | | KCPECGKSFSSKKALTEHQRTHTPNPHRRTDPSHKPFQYKCPECGKSFSTKNSLTEHQRTHTHPRAPIPKP | |
| | acaga | | FQYKCPECGKSFSQKSSLIAHQRTHTGEKPYKCPECGKSFSQSGNLTEHQRTHTHPRAPIPKPFQYKCPEC | |
| | | | GKSFSSKKHLAEHQRTHTGKKTS | |
| - | cccagagaccc | 389 | LEPGEKPYKCPECGKSFSTKNSLTEHQRTHTHPRAPIPKPFQYKCPECGKSFSQKSSLIAHQRTHTGEKPY | 871 |
| | tcaaataTcct | | KCPECGKSFSQSGNLTEHQRTHTGEKPYKCPECGKSFSTKNSLTEHQRTHTGEKPYKCPECGKSFSDPGNL | |
| | | | VRHQRTHTGEKPYKCPECGKSFSQLAHLRAHQRTHTGEKPYKCPECGKSFSSKKHLAEHQRTHTGKKTS | |
| - | agagaccctca | 390 | LEPGEKPYKCPECGKSFSSKKALTEHQRTHTPNPHRRTDPSHKPFQYKCPECGKSFSTKNSLTEHQRTHTH | 872 |
| | aataTcctCTc | | PRAPIPKPFQYKCPECGKSFSQKSSLIAHQRTHTGEKPYKCPECGKSFSQSGNLTEHQRTHTGEKPYKCPE | |
| | ac | | CGKSFSTKNSLTEHQRTHTGEKPYKCPECGKSFSDPGNLVRHQRTHTGEKPYKCPECGKSFSQLAHLRAHQ | |
| | | | RTHTGKKTS | |
| - | gaccctcaaata | 391 | LEPGEKPYKCPECGKSFSSKKALTEHQRTHTHPRAPIPKPFQYKCPECGKSFSSKKALTEHQRTHTPNPHR | 873 |
| | TcctCTcacT | | RTDPSHKPFQYKCPECGKSFSTKNSLTEHQRTHTHPRAPIPKPFQYKCPECGKSFSQKSSLIAHQRTHTGE | |
| | cac | | KPYKCPECGKSFSQSGNLTEHQRTHTGEKPYKCPECGKSFSTKNSLTEHQRTHTGEKPYKCPECGKSFSDP | |
| | | | GNLVRHQRTHTGKKTS | |
| - | cctcaaataTcc | 392 | LEPGEKPYKCPECGKSFSQLAHLRAHQRTHTGEKPYKCPECGKSFSSKKALTEHQRTHTHPRAPIPKPFQY | 874 |
| | tCTcacTcac | | KCPECGKSFSSKKALTEHQRTHTPNPHRRTDPSHKPFQYKCPECGKSFSTKNSLTEHQRTHTHPRAPIPKP | |
| | aga | | FQYKCPECGKSFSQKSSLIAHQRTHTGEKPYKCPECGKSFSQSGNLTEHQRTHTGEKPYKCPECGKSFSTK | |
| | | | NSLTEHQRTHTGKKTS | |
| - | caaataTcctC | 393 | LEPGEKPYKCPECGKSFSRRDELNVHQRTHTGEKPYKCPECGKSFSQLAHLRAHQRTHTGEKPYKCPECG | 875 |
| | TcacTcacag | | KSFSSKKALTEHQRTHTHPRAPIPKPFQYKCPECGKSFSSKKALTEHQRTHTPNPHRRTDPSHKPFQYKCP | |
| | aatg | | ECGKSFSTKNSLTEHQRTHTHPRAPIPKPFQYKCPECGKSFSQKSSLIAHQRTHTGEKPYKCPECGKSFSQ | |
| | | | SGNLTEHQRTHTGKKTS | |
| - | ataTcctCTca | 394 | LEPGEKPYKCPECGKSFSRSDELVRHQRTHTGEKPYKCPECGKSFSRRDELNVHQRTHTGEKPYKCPECG | 876 |
| | cTcacagaatg | | KSFSQLAHLRAHQRTHTGEKPYKCPECGKSFSSKKALTEHQRTHTHPRAPIPKPFQYKCPECGKSFSSKKA | |
| | gtg | | LTEHQRTHTPNPHRRTDPSHKPFQYKCPECGKSFSTKNSLTEHQRTHTHPRAPIPKPFQYKCPECGKSFSQ | |
| | | | KSSLIAHQRTHTGKKTS | |
| - | ccagagaccC | 395 | LEPGEKPYKCPECGKSFSSKKALTEHQRTHTPNPHRRTDPSHKPFQYKCPECGKSFSTKNSLTEHQRTHTH | 877 |
| | TcaaataTcct | | PRAPIPKPFQYKCPECGKSFSQKSSLIAHQRTHTGEKPYKCPECGKSFSQSGNLTEHQRTHTPNPHRRTDP | |
| | CTcac | | SHKPFQYKCPECGKSFSDKKDLTRHQRTHTGEKPYKCPECGKSFSRSDNLVRHQRTHTGEKPYKCPECGKS | |
| | | | FSTSHSLTEHQRTHTGKKTS | |
| - | gagaccCTca | 396 | LEPGEKPYKCPECGKSFSSKKALTEHQRTHTHPRAPIPKPFQYKCPECGKSFSSKKALTEHQRTHTPNPHR | 878 |
| | aataTcctCTc | | RTDPSHKPFQYKCPECGKSFSTKNSLTEHQRTHTHPRAPIPKPFQYKCPECGKSFSQKSSLIAHQRTHTGE | |
| | acTcac | | KPYKCPECGKSFSQSGNLTEHQRTHTPNPHRRTDPSHKPFQYKCPECGKSFSDKKDLTRHQRTHTGEKPYK | |
| | | | CPECGKSFSRSDNLVRHQRTHTGKKTS | |
| - | accCTcaaata | 397 | LEPGEKPYKCPECGKSFSQLAHLRAHQRTHTGEKPYKCPECGKSFSSKKALTEHQRTHTHPRAPIPKPFQY | 879 |
| | TcctCTcacT | | KCPECGKSFSSKKALTEHQRTHTPNPHRRTDPSHKPFQYKCPECGKSFSTKNSLTEHQRTHTHPRAPIPKP | |
| | cacaga | | FQYKCPECGKSFSQKSSLIAHQRTHTGEKPYKCPECGKSFSQSGNLTEHQRTHTPNPHRRTDPSHKPFQYK | |
| | | | CPECGKSFSDKKDLTRHQRTHTGKKTS | |
| - | aatATcctCT | 398 | LEPGEKPYKCPECGKSFSRSDELVRHQRTHTGEKPYKCPECGKSFSRRDELNVHQRTHTGEKPYKCPECG | 880 |
| | cacTcacagaa | | KSFSQLAHLRAHQRTHTGEKPYKCPECGKSFSSKKALTEHQRTHTHPRAPIPKPFQYKCPECGKSFSSKKA | |
| | tggtg | | LTEHQRTHTPNPHRRTDPSHKPFQYKCPECGKSFSTKNSLTEHQRTHTPNPHRRTDPSHKPFQYKCPECGK | |
| | | | SFSTTGNLTVHQRTHTGKKTS | |
| - | acagaatggTg | 399 | LEPGEKPYKCPECGKSFSDCRDLARHQRTHTGEKPYKCPECGKSFSTKNSLTEHQRTHTGEKPYKCPECG | 881 |
| | tcTctgcctgcc | | KSFSRNDALTEHQRTHTHPRAPIPKPFQYKCPECGKSFSDPGALVRHQRTHTHPRAPIPKPFQYKCPECGK | |
| | | | SFSRSDHLTTHQRTHTGEKPYKCPECGKSFSQSSNLVRHQRTHTGEKPYKCPECGKSFSSPADLTRHQRTH | |
| | | | TGKKTS | |
| - | gaatggTgtcT | 400 | LEPGEKPYKCPECGKSFSRSDKLTEHQRTHTHPRAPIPKPFQYKCPECGKSFSDCRDLARHQRTHTGEKPY | 882 |
| | ctgcctgccTc | | KCPECGKSFSTKNSLTEHQRTHTGEKPYKCPECGKSFSRNDALTEHQRTHTHPRAPIPKPFQYKCPECGKS | |
| | gg | | FSDPGALVRHQRTHTHPRAPIPKPFQYKCPECGKSFSRSDHLTTHQRTHTGEKPYKCPECGKSFSQSSNLV | |
| | | | RHQRTHTGKKTS | |
| - | tggTgtcTctg | 401 | LEPGEKPYKCPECGKSFSTSGSLVRHQRTHTGEKPYKCPECGKSFSRSDKLTEHQRTHTHPRAPIPKPFQY | 883 |
| | cctgccTcggg | | KCPECGKSFSDCRDLARHQRTHTGEKPYKCPECGKSFSTKNSLTEHQRTHTGEKPYKCPECGKSFSRNDA | |
| | tt | | LTEHQRTHTHPRAPIPKPFQYKCPECGKSFSDPGALVRHQRTHTHPRAPIPKPFQYKCPECGKSFSRSDHL | |
| | | | TTHQRTHTGKKTS | |
| - | cagaatggtgtc | 402 | LEPGEKPYKCPECGKSFSDCRDLARHQRTHTGEKPYKCPECGKSFSTKNSLTEHQRTHTGEKPYKCPECG | 884 |
| | Tctgcctgcc | | KSFSRNDALTEHQRTHTHPRAPIPKPFQYKCPECGKSFSDPGALVRHQRTHTGEKPYKCPECGKSFSTSGH | |
| | | | LVRHQRTHTGEKPYKCPECGKSFSTTGNLTVHQRTHTGEKPYKCPECGKSFSRADNLTEHQRTHTGKKTS | |
| - | aatggtgtcTct | 403 | LEPGEKPYKCPECGKSFSRSDKLTEHQRTHTHPRAPIPKPFQYKCPECGKSFSDCRDLARHQRTHTGEKPY | 885 |
| | gcctgccTcgg | | KCPECGKSFSTKNSLTEHQRTHTGEKPYKCPECGKSFSRNDALTEHQRTHTHPRAPIPKPFQYKCPECGKS | |
| | | | FSDPGALVRHQRTHTGEKPYKCPECGKSFSTSGHLVRHQRTHTGEKPYKCPECGKSFSTTGNLTVHQRTH | |
| | | | TGKKTS | |
| - | ggtgtcTctgcc | 404 | LEPGEKPYKCPECGKSFSTSGSLVRHQRTHTGEKPYKCPECGKSFSRSDKLTEHQRTHTHPRAPIPKPFQY | 886 |
| | tgccTcgggtt | | KCPECGKSFSDCRDLARHQRTHTGEKPYKCPECGKSFSTKNSLTEHQRTHTGEKPYKCPECGKSFSRNDA | |
| | | | LTEHQRTHTHPRAPIPKPFQYKCPECGKSFSDPGALVRHQRTHTGEKPYKCPECGKSFSTSGHLVRHQRTH | |
| | | | TGKKTS | |
| - | gtcTctgcctgc | 405 | LEPGEKPYKCPECGKSFSDPGHLVRHQRTHTGEKPYKCPECGKSFSTSGSLVRHQRTHTGEKPYKCPECG | 887 |
| | cTcgggttggc | | KSFSRSDKLTEHQRTHTHPRAPIPKPFQYKCPECGKSFSDCRDLARHQRTHTGEKPYKCPECGKSFSTKNS | |
| | | | LTEHQRTHTGEKPYKCPECGKSFSRNDALTEHQRTHTHPRAPIPKPFQYKCPECGKSFSDPGALVRHQRT | |
| | | | HTGKKTS | |
| - | ctgcctgccTc | 406 | LEPGEKPYKCPECGKSFSTKNSLTEHQRTHTGEKPYKCPECGKSFSDPGHLVRHQRTHTGEKPYKCPECG | 888 |
| | gggttggccct | | KSFSTSGSLVRHQRTHTGEKPYKCPECGKSFSRSDKLTEHQRTHTHPRAPIPKPFQYKCPECGKSFSDCRD | |
| | | | LARHQRTHTGEKPYKCPECGKSFSTKNSLTEHQRTHTGEKPYKCPECGKSFSRNDALTEHQRTHTGKKTS | |
| - | cctgccTcggg | 407 | LEPGEKPYKCPECGKSFSRSDELVRHQRTHTGEKPYKCPECGKSFSTKNSLTEHQRTHTGEKPYKCPECG | 889 |
| | ttggccctgtg | | KSFSDPGHLVRHQRTHTGEKPYKCPECGKSFSTSGSLVRHQRTHTGEKPYKCPECGKSFSRSDKLTEHQR | |
| | | | THTHPRAPIPKPFQYKCPECGKSFSDCRDLARHQRTHTGEKPYKCPECGKSFSTKNSLTEHQRTHTGKKTS | |
| - | gccTcgggttg | 408 | LEPGEKPYKCPECGKSFSHKNALQNHQRTHTGEKPYKCPECGKSFSRSDELVRHQRTHTGEKPYKCPECG | 890 |
| | gccctgtgatt | | KSFSTKNSLTEHQRTHTGEKPYKCPECGKSFSDPGHLVRHQRTHTGEKPYKCPECGKSFSTSGSLVRHQR | |
| | | | THTGEKPYKCPECGKSFSRSDKLTEHQRTHTHPRAPIPKPFQYKCPECGKSFSDCRDLARHQRTHTGKKTS | |
| - | gccTgccTcg | 409 | LEPGEKPYKCPECGKSFSRSDELVRHQRTHTGEKPYKCPECGKSFSTKNSLTEHQRTHTGEKPYKCPECG | 891 |
| | ggttggccctgt | | KSFSDPGHLVRHQRTHTGEKPYKCPECGKSFSTSGSLVRHQRTHTGEKPYKCPECGKSFSRSDKLTEHQR | |
| | g | | THTHPRAPIPKPFQYKCPECGKSFSDCRDLARHQRTHTHPRAPIPKPFQYKCPECGKSFSDCRDLARHQRT | |
| | | | HTGKKTS | |
| - | ctgcctcgggtt | 410 | LEPGEKPYKCPECGKSFSRSDELVRHQRTHTGEKPYKCPECGKSFSTKNSLTEHQRTHTGEKPYKCPECG | 892 |
| | ggccctgtg | | KSFSDPGHLVRHQRTHTGEKPYKCPECGKSFSTSGSLVRHQRTHTGEKPYKCPECGKSFSRSDKLTEHQR | |
| | | | THTGEKPYKCPECGKSFSTKNSLTEHQRTHTGEKPYKCPECGKSFSRNDALTEHQRTHTGKKTS | |
| - | cctcgggttggc | 411 | LEPGEKPYKCPECGKSFSHKNALQNHQRTHTGEKPYKCPECGKSFSRSDELVRHQRTHTGEKPYKCPECG | 893 |
| | cctgtgatt | | KSFSTKNSLTEHQRTHTGEKPYKCPECGKSFSDPGHLVRHQRTHTGEKPYKCPECGKSFSTSGSLVRHQR | |
| | | | THTGEKPYKCPECGKSFSRSDKLTEHQRTHTGEKPYKCPECGKSFSTKNSLTEHQRTHTGKKTS | |
| - | cgggttggccct | 412 | LEPGEKPYKCPECGKSFSHKNALQNHQRTHTHPRAPIPKPFQYKCPECGKSFSHKNALQNHQRTHTGEKP | 894 |
| | gtgattTatt | | YKCPECGKSFSRSDELVRHQRTHTGEKPYKCPECGKSFSTKNSLTEHQRTHTGEKPYKCPECGKSFSDPG | |
| | | | HLVRHQRTHTGEKPYKCPECGKSFSTSGSLVRHQRTHTGEKPYKCPECGKSFSRSDKLTEHQRTHTGKKT | |
| | | | S | |
| - | gttggccctgtg | 413 | LEPGEKPYKCPECGKSFSREDNLHTHQRTHTHPRAPIPKPFQYKCPECGKSFSHKNALQNHQRTHTHPRAP | 895 |
| | attTattTtag | | IPKPFQYKCPECGKSFSHKNALQNHQRTHTGEKPYKCPECGKSFSRSDELVRHQRTHTGEKPYKCPECGK | |
| | | | SFSTKNSLTEHQRTHTGEKPYKCPECGKSFSDPGHLVRHQRTHTGEKPYKCPECGKSFSTSGSLVRHQRT | |
| | | | HTGKKTS | |
| - | ggccctgtgatt | 414 | LEPGEKPYKCPECGKSFSTTGALTEHQRTHTPNPHRRTDPSHKPFQYKCPECGKSFSREDNLHTHQRTHTH | 896 |
| | TattTtagTTc | | PRAPIPKPFQYKCPECGKSFSHKNALQNHQRTHTHPRAPIPKPFQYKCPECGKSFSHKNALQNHQRTHTGE | |
| | tt | | KPYKCPECGKSFSRSDELVRHQRTHTGEKPYKCPECGKSFSTKNSLTEHQRTHTGEKPYKCPECGKSFSDP | |
| | | | GHLVRHQRTHTGKKTS | |
| - | cctgtgattTatt | 415 | LEPGEKPYKCPECGKSFSSKKHLAEHQRTHTPNPHRRTDPSHKPFQYKCPECGKSFSTTGALTEHQRTHTP | 897 |
| | TtagTTcttT | | NPHRRTDPSHKPFQYKCPECGKSFSREDNLHTHQRTHTHPRAPIPKPFQYKCPECGKSFSHKNALQNHQR | |
| | Tccc | | THTHPRAPIPKPFQYKCPECGKSFSHKNALQNHQRTHTGEKPYKCPECGKSFSRSDELVRHQRTHTGEKP | |
| | | | YKCPECGKSFSTKNSLTEHQRTHTGKKTS | |
| - | gtgattTattTt | 416 | LEPGEKPYKCPECGKSFSTSGSLVRHQRTHTPNPHRRTDPSHKPFQYKCPECGKSFSSKKHLAEHQRTHTP | 898 |
| | agTTcttTTc | | NPHRRTDPSHKPFQYKCPECGKSFSTTGALTEHQRTHTPNPHRRTDPSHKPFQYKCPECGKSFSREDNLHT | |
| | ccTTgtt | | HQRTHTHPRAPIPKPFQYKCPECGKSFSHKNALQNHQRTHTHPRAPIPKPFQYKCPECGKSFSHKNALQN | |
| | | | HQRTHTGEKPYKCPECGKSFSRSDELVRHQRTHTGKKTS | |
| - | gggTtggccc | 417 | LEPGEKPYKCPECGKSFSREDNLHTHQRTHTHPRAPIPKPFQYKCPECGKSFSHKNALQNHQRTHTHPRAP | 899 |
| | TgtgattTattT | | IPKPFQYKCPECGKSFSHKNALQNHQRTHTGEKPYKCPECGKSFSRSDELVRHQRTHTHPRAPIPKPFQYK | |
| | tag | | CPECGKSFSSKKHLAEHQRTHTGEKPYKCPECGKSFSRSDHLTTHQRTHTHPRAPIPKPFQYKCPECGKSF | |
| | | | SRSDKLVRHQRTHTGKKTS | |
| - | ggttggcccTg | 418 | LEPGEKPYKCPECGKSFSREDNLHTHQRTHTHPRAPIPKPFQYKCPECGKSFSHKNALQNHQRTHTHPRAP | 900 |
| | tgattTattTta | | IPKPFQYKCPECGKSFSHKNALQNHQRTHTGEKPYKCPECGKSFSRSDELVRHQRTHTHPRAPIPKPFQYK | |
| | g | | CPECGKSFSSKKHLAEHQRTHTGEKPYKCPECGKSFSRSDHLTTHQRTHTGEKPYKCPECGKSFSTSGHL | |
| | | | VRHQRTHTGKKTS | |
| - | tggcccTgtgat | 419 | LEPGEKPYKCPECGKSFSTTGALTEHQRTHTPNPHRRTDPSHKPFQYKCPECGKSFSREDNLHTHQRTHTH | 901 |
| | tTattTtagTT | | PRAPIPKPFQYKCPECGKSFSHKNALQNHQRTHTHPRAPIPKPFQYKCPECGKSFSHKNALQNHQRTHTGE | |
| | ctt | | KPYKCPECGKSFSRSDELVRHQRTHTHPRAPIPKPFQYKCPECGKSFSSKKHLAEHQRTHTGEKPYKCPEC | |
| | | | GKSFSRSDHLTTHQRTHTGKKTS | |
| - | cccTgtgattT | 420 | LEPGEKPYKCPECGKSFSSKKHLAEHQRTHTPNPHRRTDPSHKPFQYKCPECGKSFSTTGALTEHQRTHTP | 902 |
| | attTtagTTctt | | NPHRRTDPSHKPFQYKCPECGKSFSREDNLHTHQRTHTHPRAPIPKPFQYKCPECGKSFSHKNALQNHQR | |
| | TTccc | | THTHPRAPIPKPFQYKCPECGKSFSHKNALQNHQRTHTGEKPYKCPECGKSFSRSDELVRHQRTHTHPRA | |
| | | | PIPKPFQYKCPECGKSFSSKKHLAEHQRTHTGKKTS | |
| - | caaactctaTac | 421 | LEPGEKPYKCPECGKSFSQRANLRAHQRTHTPNPHRRTDPSHKPFQYKCPECGKSFSTSGSLVRHQRTHTP | 903 |
| | acttTTgttTT | | NPHRRTDPSHKPFQYKCPECGKSFSTTGALTEHQRTHTGEKPYKCPECGKSFSSPADLTRHQRTHTHPRAP | |
| | aaa | | IPKPFQYKCPECGKSFSQNSTLTEHQRTHTGEKPYKCPECGKSFSTHLDLIRHQRTHTGEKPYKCPECGKS | |
| | | | FSQSGNLTEHQRTHTGKKTS | |
| - | actctaTacact | 422 | LEPGEKPYKCPECGKSFSDSGNLRVHQRTHTGEKPYKCPECGKSFSQRANLRAHQRTHTPNPHRRTDPSH | 904 |
| | tTTgttTTaaa | | KPFQYKCPECGKSFSTSGSLVRHQRTHTPNPHRRTDPSHKPFQYKCPECGKSFSTTGALTEHQRTHTGEKP | |
| | aac | | YKCPECGKSFSSPADLTRHQRTHTHPRAPIPKPFQYKCPECGKSFSQNSTLTEHQRTHTGEKPYKCPECGK | |
| | | | SFSTHLDLIRHQRTHTGKKTS | |
| - | ctaTacacttT | 423 | LEPGEKPYKCPECGKSFSRSDELVRHQRTHTHPRAPIPKPFQYKCPECGKSFSDSGNLRVHQRTHTGEKPY | 905 |
| | TgttTTaaaaa | | KCPECGKSFSQRANLRAHQRTHTPNPHRRTDPSHKPFQYKCPECGKSFSTSGSLVRHQRTHTPNPHRRTD | |
| | cTgtg | | PSHKPFQYKCPECGKSFSTTGALTEHQRTHTGEKPYKCPECGKSFSSPADLTRHQRTHTHPRAPIPKPFQY | |
| | | | KCPECGKSFSQNSTLTEHQRTHTGKKTS | |
| - | aaaCTctaTa | 424 | LEPGEKPYKCPECGKSFSDSGNLRVHQRTHTGEKPYKCPECGKSFSQRANLRAHQRTHTPNPHRRTDPSH | 906 |
| | cacttTTgttT | | KPFQYKCPECGKSFSTSGSLVRHQRTHTPNPHRRTDPSHKPFQYKCPECGKSFSTTGALTEHQRTHTGEKP | |
| | Taaaaac | | YKCPECGKSFSSPADLTRHQRTHTHPRAPIPKPFQYKCPECGKSFSQNSTLTEHQRTHTPNPHRRTDPSHK | |
| | | | PFQYKCPECGKSFSQRANLRAHQRTHTGKKTS | |
| - | aacTctaTaca | 425 | LEPGEKPYKCPECGKSFSDSGNLRVHQRTHTGEKPYKCPECGKSFSQRANLRAHQRTHTPNPHRRTDPSH | 907 |
| | cttTTgttTTa | | KPFQYKCPECGKSFSTSGSLVRHQRTHTPNPHRRTDPSHKPFQYKCPECGKSFSTTGALTEHQRTHTGEKP | |
| | aaaac | | YKCPECGKSFSSPADLTRHQRTHTHPRAPIPKPFQYKCPECGKSFSQNSTLTEHQRTHTHPRAPIPKPFQY | |
| | | | KCPECGKSFSDSGNLRVHQRTHTGKKTS | |
| - | acacttTTgttT | 426 | LEPGEKPYKCPECGKSFSTSGSLVRHQRTHTGEKPYKCPECGKSFSRSDELVRHQRTHTHPRAPIPKPFQY | 908 |
| | TaaaaacTgtg | | KCPECGKSFSDSGNLRVHQRTHTGEKPYKCPECGKSFSQRANLRAHQRTHTPNPHRRTDPSHKPFQYKCP | |
| | gtt | | ECGKSFSTSGSLVRHQRTHTPNPHRRTDPSHKPFQYKCPECGKSFSTTGALTEHQRTHTGEKPYKCPECG | |
| | | | KSFSSPADLTRHQRTHTGKKTS | |
| - | atacctCTcac | 427 | LEPGEKPYKCPECGKSFSRSDKLVRHQRTHTGEKPYKCPECGKSFSQAGHLASHQRTHTGEKPYKCPECG | 909 |
| | CTctgtggtga | | KSFSRSDHLTTHQRTHTGEKPYKCPECGKSFSRNDALTEHQRTHTPNPHRRTDPSHKPFQYKCPECGKSFS | |
| | ggg | | SKKALTEHQRTHTPNPHRRTDPSHKPFQYKCPECGKSFSTKNSLTEHQRTHTGEKPYKCPECGKSFSQKSS | |
| | | | LIAHQRTHTGKKTS | |
| - | cctCTcacCT | 428 | LEPGEKPYKCPECGKSFSQSSNLVRHQRTHTGEKPYKCPECGKSFSRSDKLVRHQRTHTGEKPYKCPECG | 910 |
| | ctgtggtgagg | | KSFSQAGHLASHQRTHTGEKPYKCPECGKSFSRSDHLTTHQRTHTGEKPYKCPECGKSFSRNDALTEHQR | |
| | ggaa | | THTPNPHRRTDPSHKPFQYKCPECGKSFSSKKALTEHQRTHTPNPHRRTDPSHKPFQYKCPECGKSFSTKN | |
| | | | SLTEHQRTHTGKKTS | |
| - | cacCTctgtgg | 429 | LEPGEKPYKCPECGKSFSQSSNLVRHQRTHTGEKPYKCPECGKSFSQSSNLVRHQRTHTGEKPYKCPECG | 911 |
| | tgaggggaaga | | KSFSRSDKLVRHQRTHTGEKPYKCPECGKSFSQAGHLASHQRTHTGEKPYKCPECGKSFSRSDHLTTHQR | |
| | a | | THTGEKPYKCPECGKSFSRNDALTEHQRTHTPNPHRRTDPSHKPFQYKCPECGKSFSSKKALTEHQRTHT | |
| | | | GKKTS | |
| - | accTctgtggtg | 430 | LEPGEKPYKCPECGKSFSQSSNLVRHQRTHTGEKPYKCPECGKSFSQSSNLVRHQRTHTGEKPYKCPECG | 912 |
| | aggggaagaa | | KSFSRSDKLVRHQRTHTGEKPYKCPECGKSFSQAGHLASHQRTHTGEKPYKCPECGKSFSRSDHLTTHQR | |
| | | | THTGEKPYKCPECGKSFSRNDALTEHQRTHTHPRAPIPKPFQYKCPECGKSFSDKKDLTRHQRTHTGKKT | |
| | | | S | |
| - | cctctgtggtga | 431 | LEPGEKPYKCPECGKSFSQSSNLVRHQRTHTGEKPYKCPECGKSFSQSSNLVRHQRTHTGEKPYKCPECG | 913 |
| | ggggaagaa | | KSFSRSDKLVRHQRTHTGEKPYKCPECGKSFSQAGHLASHQRTHTGEKPYKCPECGKSFSRSDHLTTHQR | |
| | | | THTGEKPYKCPECGKSFSRNDALTEHQRTHTGEKPYKCPECGKSFSTKNSLTEHQRTHTGKKTS | |
| - | ctgtggtgagg | 432 | LEPGEKPYKCPECGKSFSTSGNLTEHQRTHTPNPHRRTDPSHKPFQYKCPECGKSFSQSSNLVRHQRTHTG | 914 |
| | ggaagaaATc | | EKPYKCPECGKSFSQSSNLVRHQRTHTGEKPYKCPECGKSFSRSDKLVRHQRTHTGEKPYKCPECGKSFS | |
| | at | | QAGHLASHQRTHTGEKPYKCPECGKSFSRSDHLTTHQRTHTGEKPYKCPECGKSFSRNDALTEHQRTHT | |
| | | | GKKTS | |
| - | tggtgagggga | 433 | LEPGEKPYKCPECGKSFSHKNALQNHQRTHTGEKPYKCPECGKSFSTSGNLTEHQRTHTPNPHRRTDPSH | 915 |
| | agaaATcatat | | KPFQYKCPECGKSFSQSSNLVRHQRTHTGEKPYKCPECGKSFSQSSNLVRHQRTHTGEKPYKCPECGKSF | |
| | t | | SRSDKLVRHQRTHTGEKPYKCPECGKSFSQAGHLASHQRTHTGEKPYKCPECGKSFSRSDHLTTHQRTHT | |
| | | | GKKTS | |
| - | tgaggggaaga | 434 | LEPGEKPYKCPECGKSFSRADNLTEHQRTHTPNPHRRTDPSHKPFQYKCPECGKSFSHKNALQNHQRTHT | 916 |
| | aATcatattTT | | GEKPYKCPECGKSFSTSGNLTEHQRTHTPNPHRRTDPSHKPFQYKCPECGKSFSQSSNLVRHQRTHTGEKP | |
| | cag | | YKCPECGKSFSQSSNLVRHQRTHTGEKPYKCPECGKSFSRSDKLVRHQRTHTGEKPYKCPECGKSFSQAG | |
| | | | HLASHQRTHTGKKTS | |
| - | ggggaagaaA | 435 | LEPGEKPYKCPECGKSFSRRDELNVHQRTHTGEKPYKCPECGKSFSRADNLTEHQRTHTPNPHRRTDPSH | 917 |
| | TcatattTTca | | KPFQYKCPECGKSFSHKNALQNHQRTHTGEKPYKCPECGKSFSTSGNLTEHQRTHTPNPHRRTDPSHKPF | |
| | gatg | | QYKCPECGKSFSQSSNLVRHQRTHTGEKPYKCPECGKSFSQSSNLVRHQRTHTGEKPYKCPECGKSFSRS | |
| | | | DKLVRHQRTHTGKKTS | |
| - | gaagaaATca | 436 | LEPGEKPYKCPECGKSFSTHLDLIRHQRTHTGEKPYKCPECGKSFSRRDELNVHQRTHTGEKPYKCPECG | 918 |
| | tattTTcagatg | | KSFSRADNLTEHQRTHTPNPHRRTDPSHKPFQYKCPECGKSFSHKNALQNHQRTHTGEKPYKCPECGKSF | |
| | act | | STSGNLTEHQRTHTPNPHRRTDPSHKPFQYKCPECGKSFSQSSNLVRHQRTHTGEKPYKCPECGKSFSQSS | |
| | | | NLVRHQRTHTGKKTS | |
| - | gaaATcatatt | 437 | LEPGEKPYKCPECGKSFSSRRTCRAHQRTHTGEKPYKCPECGKSFSTHLDLIRHQRTHTGEKPYKCPECGK | 919 |
| | TTcagatgact | | SFSRRDELNVHQRTHTGEKPYKCPECGKSFSRADNLTEHQRTHTPNPHRRTDPSHKPFQYKCPECGKSFS | |
| | cgt | | HKNALQNHQRTHTGEKPYKCPECGKSFSTSGNLTEHQRTHTPNPHRRTDPSHKPFQYKCPECGKSFSQSS | |
| | | | NLVRHQRTHTGKKTS | |
| - | gtggtgagggg | 438 | LEPGEKPYKCPECGKSFSTSGNLTEHQRTHTGEKPYKCPECGKSFSTTGNLTVHQRTHTGEKPYKCPECG | 920 |
| | aagaaatcat | | KSFSQLAHLRAHQRTHTGEKPYKCPECGKSFSQRAHLERHQRTHTGEKPYKCPECGKSFSRSDHLTNHQR | |
| | | | THTGEKPYKCPECGKSFSRSDELVRHQRTHTGEKPYKCPECGKSFSRSDELVRHQRTHTGKKTS | |
| - | gtgaggggaag | 439 | LEPGEKPYKCPECGKSFSHKNALQNHQRTHTGEKPYKCPECGKSFSTSGNLTEHQRTHTGEKPYKCPECG | 921 |
| | aaatcatatt | | KSFSTTGNLTVHQRTHTGEKPYKCPECGKSFSQLAHLRAHQRTHTGEKPYKCPECGKSFSQRAHLERHQR | |
| | | | THTGEKPYKCPECGKSFSRSDHLTNHQRTHTGEKPYKCPECGKSFSRSDELVRHQRTHTGKKTS | |
| - | aggggaagaaa | 440 | LEPGEKPYKCPECGKSFSRADNLTEHQRTHTPNPHRRTDPSHKPFQYKCPECGKSFSHKNALQNHQRTHT | 922 |
| | tcatattTTcag | | GEKPYKCPECGKSFSTSGNLTEHQRTHTGEKPYKCPECGKSFSTTGNLTVHQRTHTGEKPYKCPECGKSF | |
| | | | SQLAHLRAHQRTHTGEKPYKCPECGKSFSQRAHLERHQRTHTGEKPYKCPECGKSFSRSDHLTNHQRTH | |
| | | | TGKKTS | |
| - | ggaagaaatcat | 441 | LEPGEKPYKCPECGKSFSRRDELNVHQRTHTGEKPYKCPECGKSFSRADNLTEHQRTHTPNPHRRTDPSH | 923 |
| | attTTcagatg | | KPFQYKCPECGKSFSHKNALQNHQRTHTGEKPYKCPECGKSFSTSGNLTEHQRTHTGEKPYKCPECGKSF | |
| | | | STTGNLTVHQRTHTGEKPYKCPECGKSFSQLAHLRAHQRTHTGEKPYKCPECGKSFSQRAHLERHQRTHT | |
| | | | GKKTS | |
| - | agaaatcatatt | 442 | LEPGEKPYKCPECGKSFSTHLDLIRHQRTHTGEKPYKCPECGKSFSRRDELNVHQRTHTGEKPYKCPECG | 924 |
| | TTcagatgact | | KSFSRADNLTEHQRTHTPNPHRRTDPSHKPFQYKCPECGKSFSHKNALQNHQRTHTGEKPYKCPECGKSF | |
| | | | STSGNLTEHQRTHTGEKPYKCPECGKSFSTTGNLTVHQRTHTGEKPYKCPECGKSFSQLAHLRAHQRTHT | |
| | | | GKKTS | |
| - | aatcatattTTc | 443 | LEPGEKPYKCPECGKSFSSRRTCRAHQRTHTGEKPYKCPECGKSFSTHLDLIRHQRTHTGEKPYKCPECGK | 925 |
| | agatgactcgt | | SFSRRDELNVHQRTHTGEKPYKCPECGKSFSRADNLTEHQRTHTPNPHRRTDPSHKPFQYKCPECGKSFS | |
| | | | HKNALQNHQRTHTGEKPYKCPECGKSFSTSGNLTEHQRTHTGEKPYKCPECGKSFSTTGNLTVHQRTHT | |
| | | | GKKTS | |
| - | catattTTcaga | 444 | LEPGEKPYKCPECGKSFSQRANLRAHQRTHTGEKPYKCPECGKSFSSRRTCRAHQRTHTGEKPYKCPECG | 926 |
| | tgactcgtaaa | | KSFSTHLDLIRHQRTHTGEKPYKCPECGKSFSRRDELNVHQRTHTGEKPYKCPECGKSFSRADNLTEHQR | |
| | | | THTPNPHRRTDPSHKPFQYKCPECGKSFSHKNALQNHQRTHTGEKPYKCPECGKSFSTSGNLTEHQRTHT | |
| | | | GKKTS | |
| - | attTTcagatg | 445 | LEPGEKPYKCPECGKSFSRSDKLVRHQRTHTGEKPYKCPECGKSFSQRANLRAHQRTHTGEKPYKCPECG | 927 |
| | actcgtaaaggg | | KSFSSRRTCRAHQRTHTGEKPYKCPECGKSFSTHLDLIRHQRTHTGEKPYKCPECGKSFSRRDELNVHQRT | |
| | | | HTGEKPYKCPECGKSFSRADNLTEHQRTHTPNPHRRTDPSHKPFQYKCPECGKSFSHKNALQNHQRTHTG | |
| | | | KKTS | |
| - | ggtgaggggaa | 446 | LEPGEKPYKCPECGKSFSHKNALQNHQRTHTGEKPYKCPECGKSFSTSGNLTEHQRTHTHPRAPIPKPFQY | 928 |
| | gaaaTcatatt | | KCPECGKSFSQRANLRAHQRTHTGEKPYKCPECGKSFSRKDNLKNHQRTHTGEKPYKCPECGKSFSRSD | |
| | | | KLVRHQRTHTGEKPYKCPECGKSFSRSDNLVRHQRTHTGEKPYKCPECGKSFSTSGHLVRHQRTHTGKK | |
| | | | TS | |
| - | gaggggaagaa | 447 | LEPGEKPYKCPECGKSFSRADNLTEHQRTHTPNPHRRTDPSHKPFQYKCPECGKSFSHKNALQNHQRTHT | 929 |
| | aTcatattTTc | | GEKPYKCPECGKSFSTSGNLTEHQRTHTHPRAPIPKPFQYKCPECGKSFSQRANLRAHQRTHTGEKPYKCP | |
| | ag | | ECGKSFSRKDNLKNHQRTHTGEKPYKCPECGKSFSRSDKLVRHQRTHTGEKPYKCPECGKSFSRSDNLVR | |
| | | | HQRTHTGKKTS | |
| - | gggaagaaaT | 448 | LEPGEKPYKCPECGKSFSRRDELNVHQRTHTGEKPYKCPECGKSFSRADNLTEHQRTHTPNPHRRTDPSH | 930 |
| | catattTTcaga | | KPFQYKCPECGKSFSHKNALQNHQRTHTGEKPYKCPECGKSFSTSGNLTEHQRTHTHPRAPIPKPFQYKCP | |
| | tg | | ECGKSFSQRANLRAHQRTHTGEKPYKCPECGKSFSRKDNLKNHQRTHTGEKPYKCPECGKSFSRSDKLV | |
| | | | RHQRTHTGKKTS | |
| - | aagaaaTcatat | 449 | LEPGEKPYKCPECGKSFSTHLDLIRHQRTHTGEKPYKCPECGKSFSRRDELNVHQRTHTGEKPYKCPECG | 931 |
| | tTTcagatgac | | KSFSRADNLTEHQRTHTPNPHRRTDPSHKPFQYKCPECGKSFSHKNALQNHQRTHTGEKPYKCPECGKSF | |
| | t | | STSGNLTEHQRTHTHPRAPIPKPFQYKCPECGKSFSQRANLRAHQRTHTGEKPYKCPECGKSFSRKDNLK | |
| | | | NHQRTHTGKKTS | |
| - | aaaTcatattT | 450 | LEPGEKPYKCPECGKSFSSRRTCRAHQRTHTGEKPYKCPECGKSFSTHLDLIRHQRTHTGEKPYKCPECGK | 932 |
| | Tcagatgactc | | SFSRRDELNVHQRTHTGEKPYKCPECGKSFSRADNLTEHQRTHTPNPHRRTDPSHKPFQYKCPECGKSFS | |
| | gt | | HKNALQNHQRTHTGEKPYKCPECGKSFSTSGNLTEHQRTHTHPRAPIPKPFQYKCPECGKSFSQRANLRA | |
| | | | HQRTHTGKKTS | |
| - | cagatgactcgt | 451 | LEPGEKPYKCPECGKSFSQSGNLTEHQRTHTGEKPYKCPECGKSFSRSDKLVRHQRTHTGEKPYKCPECG | 933 |
| | aaagggcaa | | KSFSQRANLRAHQRTHTGEKPYKCPECGKSFSSRRTCRAHQRTHTGEKPYKCPECGKSFSTHLDLIRHQR | |
| | | | THTGEKPYKCPECGKSFSRRDELNVHQRTHTGEKPYKCPECGKSFSRADNLTEHQRTHTGKKTS | |
| - | atgactcgtaaa | 452 | LEPGEKPYKCPECGKSFSQLAHLRAHQRTHTGEKPYKCPECGKSFSQSGNLTEHQRTHTGEKPYKCPECG | 934 |
| | gggcaaaga | | KSFSRSDKLVRHQRTHTGEKPYKCPECGKSFSQRANLRAHQRTHTGEKPYKCPECGKSFSSRRTCRAHQR | |
| | | | THTGEKPYKCPECGKSFSTHLDLIRHQRTHTGEKPYKCPECGKSFSRRDELNVHQRTHTGKKTS | |
| - | actcgtaaaggg | 453 | LEPGEKPYKCPECGKSFSQRANLRAHQRTHTGEKPYKCPECGKSFSQLAHLRAHQRTHTGEKPYKCPECG | 935 |
| | caaagaaaa | | KSFSQSGNLTEHQRTHTGEKPYKCPECGKSFSRSDKLVRHQRTHTGEKPYKCPECGKSFSQRANLRAHQR | |
| | | | THTGEKPYKCPECGKSFSSRRTCRAHQRTHTGEKPYKCPECGKSFSTHLDLIRHQRTHTGKKTS | |
| - | cgtaaagggca | 454 | LEPGEKPYKCPECGKSFSQRANLRAHQRTHTGEKPYKCPECGKSFSQRANLRAHQRTHTGEKPYKCPEC | 936 |
| | aagaaaaaaa | | GKSFSQLAHLRAHQRTHTGEKPYKCPECGKSFSQSGNLTEHQRTHTGEKPYKCPECGKSFSRSDKLVRHQ | |
| | | | RTHTGEKPYKCPECGKSFSQRANLRAHQRTHTGEKPYKCPECGKSFSSRRTCRAHQRTHTGKKTS | |
| - | aaagggcaaag | 455 | LEPGEKPYKCPECGKSFSSKKHLAEHQRTHTGEKPYKCPECGKSFSQRANLRAHQRTHTGEKPYKCPECG | 937 |
| | aaaaaaaccc | | KSFSQRANLRAHQRTHTGEKPYKCPECGKSFSQLAHLRAHQRTHTGEKPYKCPECGKSFSQSGNLTEHQR | |
| | | | THTGEKPYKCPECGKSFSRSDKLVRHQRTHTGEKPYKCPECGKSFSQRANLRAHQRTHTGKKTS | |
| - | gggcaaagaaa | 456 | LEPGEKPYKCPECGKSFSQRANLRAHQRTHTGEKPYKCPECGKSFSSKKHLAEHQRTHTGEKPYKCPECG | 938 |
| | aaaacccaaa | | KSFSQRANLRAHQRTHTGEKPYKCPECGKSFSQRANLRAHQRTHTGEKPYKCPECGKSFSQLAHLRAHQ | |
| | | | RTHTGEKPYKCPECGKSFSQSGNLTEHQRTHTGEKPYKCPECGKSFSRSDKLVRHQRTHTGKKTS | |
| - | caaagaaaaaa | 457 | LEPGEKPYKCPECGKSFSHKNALQNHQRTHTGEKPYKCPECGKSFSQRANLRAHQRTHTGEKPYKCPEC | 939 |
| | acccaaaatt | | GKSFSSKKHLAEHQRTHTGEKPYKCPECGKSFSQRANLRAHQRTHTGEKPYKCPECGKSFSQRANLRAH | |
| | | | QRTHTGEKPYKCPECGKSFSQLAHLRAHQRTHTGEKPYKCPECGKSFSQSGNLTEHQRTHTGKKTS | |
| - | agaaaaaaacc | 458 | LEPGEKPYKCPECGKSFSQSGNLTEHQRTHTHPRAPIPKPFQYKCPECGKSFSHKNALQNHQRTHTGEKPY | 940 |
| | caaaattTcaa | | KCPECGKSFSQRANLRAHQRTHTGEKPYKCPECGKSFSSKKHLAEHQRTHTGEKPYKCPECGKSFSQRAN | |
| | | | LRAHQRTHTGEKPYKCPECGKSFSQRANLRAHQRTHTGEKPYKCPECGKSFSQLAHLRAHQRTHTGKKT | |
| | | | s | |
| - | aaaaaacccaa | 459 | LEPGEKPYKCPECGKSFSTTGNLTVHQRTHTGEKPYKCPECGKSFSQSGNLTEHQRTHTHPRAPIPKPFQY | 941 |
| | aattTcaaaat | | KCPECGKSFSHKNALQNHQRTHTGEKPYKCPECGKSFSQRANLRAHQRTHTGEKPYKCPECGKSFSSKK | |
| | | | HLAEHQRTHTGEKPYKCPECGKSFSQRANLRAHQRTHTGEKPYKCPECGKSFSQRANLRAHQRTHTGKK | |
| | | | TS | |
| - | aaacccaaaatt | 460 | LEPGEKPYKCPECGKSFSRNDTLTEHQRTHTPNPHRRTDPSHKPFQYKCPECGKSFSTTGNLTVHQRTHTG | 942 |
| | TcaaaatTTcc | | EKPYKCPECGKSFSQSGNLTEHQRTHTHPRAPIPKPFQYKCPECGKSFSHKNALQNHQRTHTGEKPYKCP | |
| | g | | ECGKSFSQRANLRAHQRTHTGEKPYKCPECGKSFSSKKHLAEHQRTHTGEKPYKCPECGKSFSQRANLR | |
| | | | AHQRTHTGKKTS | |
| - | agatgaCTcgt | 461 | LEPGEKPYKCPECGKSFSQLAHLRAHQRTHTGEKPYKCPECGKSFSQSGNLTEHQRTHTGEKPYKCPECG | 943 |
| | aaagggcaaag | | KSFSRSDKLVRHQRTHTGEKPYKCPECGKSFSQRANLRAHQRTHTGEKPYKCPECGKSFSSRRTCRAHQR | |
| | a | | THTPNPHRRTDPSHKPFQYKCPECGKSFSQAGHLASHQRTHTGEKPYKCPECGKSFSQLAHLRAHQRTHT | |
| | | | GKKTS | |
| - | tgaCTcgtaaa | 462 | LEPGEKPYKCPECGKSFSQRANLRAHQRTHTGEKPYKCPECGKSFSQLAHLRAHQRTHTGEKPYKCPECG | 944 |
| | gggcaaagaaa | | KSFSQSGNLTEHQRTHTGEKPYKCPECGKSFSRSDKLVRHQRTHTGEKPYKCPECGKSFSQRANLRAHQR | |
| | a | | THTGEKPYKCPECGKSFSSRRTCRAHQRTHTPNPHRRTDPSHKPFQYKCPECGKSFSQAGHLASHQRTHT | |
| | | | GKKTS | |
| - | gatgacTcgta | 463 | LEPGEKPYKCPECGKSFSQLAHLRAHQRTHTGEKPYKCPECGKSFSQSGNLTEHQRTHTGEKPYKCPECG | 945 |
| | aagggcaaaga | | KSFSRSDKLVRHQRTHTGEKPYKCPECGKSFSQRANLRAHQRTHTGEKPYKCPECGKSFSSRRTCRAHQR | |
| | | | THTHPRAPIPKPFQYKCPECGKSFSDPGNLVRHQRTHTGEKPYKCPECGKSFSTSGNLVRHQRTHTGKKTS | |
| - | gacTcgtaaag | 464 | LEPGEKPYKCPECGKSFSQRANLRAHQRTHTGEKPYKCPECGKSFSQLAHLRAHQRTHTGEKPYKCPECG | 946 |
| | ggcaaagaaaa | | KSFSQSGNLTEHQRTHTGEKPYKCPECGKSFSRSDKLVRHQRTHTGEKPYKCPECGKSFSQRANLRAHQR | |
| | | | THTGEKPYKCPECGKSFSSRRTCRAHQRTHTHPRAPIPKPFQYKCPECGKSFSDPGNLVRHQRTHTGKKTS | |
| - | gtaaagggcaa | 465 | LEPGEKPYKCPECGKSFSDSGNLRVHQRTHTGEKPYKCPECGKSFSQRANLRAHQRTHTGEKPYKCPECG | 947 |
| | agaaaaaaac | | KSFSQSSNLVRHQRTHTGEKPYKCPECGKSFSQRANLRAHQRTHTGEKPYKCPECGKSFSDPGHLVRHQR | |
| | | | THTGEKPYKCPECGKSFSRKDNLKNHQRTHTGEKPYKCPECGKSFSQSSSLVRHQRTHTGKKTS | |
| - | aagggcaaaga | 466 | LEPGEKPYKCPECGKSFSTSHSLTEHQRTHTGEKPYKCPECGKSFSDSGNLRVHQRTHTGEKPYKCPECG | 948 |
| | aaaaaaccca | | KSFSQRANLRAHQRTHTGEKPYKCPECGKSFSQSSNLVRHQRTHTGEKPYKCPECGKSFSQRANLRAHQ | |
| | | | RTHTGEKPYKCPECGKSFSDPGHLVRHQRTHTGEKPYKCPECGKSFSRKDNLKNHQRTHTGKKTS | |
| - | ggcaaagaaaa | 467 | LEPGEKPYKCPECGKSFSQRANLRAHQRTHTGEKPYKCPECGKSFSTSHSLTEHQRTHTGEKPYKCPECG | 949 |
| | aaacccaaaa | | KSFSDSGNLRVHQRTHTGEKPYKCPECGKSFSQRANLRAHQRTHTGEKPYKCPECGKSFSQSSNLVRHQR | |
| | | | THTGEKPYKCPECGKSFSQRANLRAHQRTHTGEKPYKCPECGKSFSDPGHLVRHQRTHTGKKTS | |
| - | agggcaaagaa | 468 | LEPGEKPYKCPECGKSFSQSGNLTEHQRTHTGEKPYKCPECGKSFSDKKDLTRHQRTHTGEKPYKCPECG | 950 |
| | aaaaacccaa | | KSFSQRANLRAHQRTHTGEKPYKCPECGKSFSQRANLRAHQRTHTGEKPYKCPECGKSFSRKDNLKNHQ | |
| | | | RTHTGEKPYKCPECGKSFSQSGDLRRHQRTHTGEKPYKCPECGKSFSRSDHLTNHQRTHTGKKTS | |
| - | gcaaagaaaaa | 469 | LEPGEKPYKCPECGKSFSTTGNLTVHQRTHTGEKPYKCPECGKSFSQSGNLTEHQRTHTGEKPYKCPECG | 951 |
| | aacccaaaat | | KSFSDKKDLTRHQRTHTGEKPYKCPECGKSFSQRANLRAHQRTHTGEKPYKCPECGKSFSQRANLRAHQ | |
| | | | RTHTGEKPYKCPECGKSFSRKDNLKNHQRTHTGEKPYKCPECGKSFSQSGDLRRHQRTHTGKKTS | |
| - | aagaaaaaaac | 470 | LEPGEKPYKCPECGKSFSQSGNLTEHQRTHTPNPHRRTDPSHKPFQYKCPECGKSFSTTGNLTVHQRTHTG | 952 |
| | ccaaaatTTca | | EKPYKCPECGKSFSQSGNLTEHQRTHTGEKPYKCPECGKSFSDKKDLTRHQRTHTGEKPYKCPECGKSFS | |
| | a | | QRANLRAHQRTHTGEKPYKCPECGKSFSQRANLRAHQRTHTGEKPYKCPECGKSFSRKDNLKNHQRTHT | |
| | | | GKKTS | |
| - | aaaaaaaccca | 471 | LEPGEKPYKCPECGKSFSTTGNLTVHQRTHTGEKPYKCPECGKSFSQSGNLTEHQRTHTPNPHRRTDPSH | 953 |
| | aaatTTcaaaa | | KPFQYKCPECGKSFSTTGNLTVHQRTHTGEKPYKCPECGKSFSQSGNLTEHQRTHTGEKPYKCPECGKSF | |
| | t | | SDKKDLTRHQRTHTGEKPYKCPECGKSFSQRANLRAHQRTHTGEKPYKCPECGKSFSQRANLRAHQRTH | |
| | | | TGKKTS | |
| - | aaaacccaaaat | 472 | LEPGEKPYKCPECGKSFSRNDTLTEHQRTHTPNPHRRTDPSHKPFQYKCPECGKSFSTTGNLTVHQRTHTG | 954 |
| | TTcaaaatTT | | EKPYKCPECGKSFSQSGNLTEHQRTHTPNPHRRTDPSHKPFQYKCPECGKSFSTTGNLTVHQRTHTGEKP | |
| | ccg | | YKCPECGKSFSQSGNLTEHQRTHTGEKPYKCPECGKSFSDKKDLTRHQRTHTGEKPYKCPECGKSFSQRA | |
| | | | NLRAHQRTHTGKKTS | |
| - | agtCTcataat | 473 | LEPGEKPYKCPECGKSFSRSDNLVRHQRTHTGEKPYKCPECGKSFSRKDNLKNHQRTHTGEKPYKCPECG | 955 |
| | caagaaaagga | | KSFSQSSNLVRHQRTHTGEKPYKCPECGKSFSQSGNLTEHQRTHTGEKPYKCPECGKSFSTTGNLTVHQR | |
| | g | | THTGEKPYKCPECGKSFSTSGNLTEHQRTHTPNPHRRTDPSHKPFQYKCPECGKSFSHRTTLTNHQRTHTG | |
| | | | KKTS | |
| - | gtcTcataatca | 474 | LEPGEKPYKCPECGKSFSRSDNLVRHQRTHTGEKPYKCPECGKSFSRKDNLKNHQRTHTGEKPYKCPECG | 956 |
| | agaaaaggag | | KSFSQSSNLVRHQRTHTGEKPYKCPECGKSFSQSGNLTEHQRTHTGEKPYKCPECGKSFSTTGNLTVHQR | |
| | | | THTGEKPYKCPECGKSFSTSGNLTEHQRTHTHPRAPIPKPFQYKCPECGKSFSDPGALVRHQRTHTGKKTS | |
| - | cataatcaagaa | 475 | LEPGEKPYKCPECGKSFSQRANLRAHQRTHTGEKPYKCPECGKSFSRSDNLVRHQRTHTGEKPYKCPECG | 957 |
| | aaggagaaa | | KSFSRKDNLKNHQRTHTGEKPYKCPECGKSFSQSSNLVRHQRTHTGEKPYKCPECGKSFSQSGNLTEHQR | |
| | | | THTGEKPYKCPECGKSFSTTGNLTVHQRTHTGEKPYKCPECGKSFSTSGNLTEHQRTHTGKKTS | |
| - | aatcaagaaaa | 476 | LEPGEKPYKCPECGKSFSSKKALTEHQRTHTGEKPYKCPECGKSFSQRANLRAHQRTHTGEKPYKCPECG | 958 |
| | ggagaaacac | | KSFSRSDNLVRHQRTHTGEKPYKCPECGKSFSRKDNLKNHQRTHTGEKPYKCPECGKSFSQSSNLVRHQR | |
| | | | THTGEKPYKCPECGKSFSQSGNLTEHQRTHTGEKPYKCPECGKSFSTTGNLTVHQRTHTGKKTS | |
| - | caagaaaagga | 477 | LEPGEKPYKCPECGKSFSQLAHLRAHQRTHTGEKPYKCPECGKSFSSKKALTEHQRTHTGEKPYKCPECG | 959 |
| | gaaacacaga | | KSFSQRANLRAHQRTHTGEKPYKCPECGKSFSRSDNLVRHQRTHTGEKPYKCPECGKSFSRKDNLKNHQ | |
| | | | RTHTGEKPYKCPECGKSFSQSSNLVRHQRTHTGEKPYKCPECGKSFSQSGNLTEHQRTHTGKKTS | |
| - | gaaaaggagaa | 478 | LEPGEKPYKCPECGKSFSRSDNLVRHQRTHTGEKPYKCPECGKSFSQLAHLRAHQRTHTGEKPYKCPECG | 960 |
| | acacagagag | | KSFSSKKALTEHQRTHTGEKPYKCPECGKSFSQRANLRAHQRTHTGEKPYKCPECGKSFSRSDNLVRHQR | |
| | | | THTGEKPYKCPECGKSFSRKDNLKNHQRTHTGEKPYKCPECGKSFSQSSNLVRHQRTHTGKKTS | |
| - | aaggagaaaca | 479 | LEPGEKPYKCPECGKSFSQLAHLRAHQRTHTGEKPYKCPECGKSFSRSDNLVRHQRTHTGEKPYKCPECG | 961 |
| | cagagagaga | | KSFSQLAHLRAHQRTHTGEKPYKCPECGKSFSSKKALTEHQRTHTGEKPYKCPECGKSFSQRANLRAHQR | |
| | | | THTGEKPYKCPECGKSFSRSDNLVRHQRTHTGEKPYKCPECGKSFSRKDNLKNHQRTHTGKKTS | |
| - | gagaaacacag | 480 | LEPGEKPYKCPECGKSFSQSSNLVRHQRTHTGEKPYKCPECGKSFSQLAHLRAHQRTHTGEKPYKCPECG | 962 |
| | agagagagaa | | KSFSRSDNLVRHQRTHTGEKPYKCPECGKSFSQLAHLRAHQRTHTGEKPYKCPECGKSFSSKKALTEHQR | |
| | | | THTGEKPYKCPECGKSFSQRANLRAHQRTHTGEKPYKCPECGKSFSRSDNLVRHQRTHTGKKTS | |
| - | aaacacagaga | 481 | LEPGEKPYKCPECGKSFSQRANLRAHQRTHTGEKPYKCPECGKSFSQSSNLVRHQRTHTGEKPYKCPECG | 963 |
| | gagagaaaaa | | KSFSQLAHLRAHQRTHTGEKPYKCPECGKSFSRSDNLVRHQRTHTGEKPYKCPECGKSFSQLAHLRAHQ | |
| | | | RTHTGEKPYKCPECGKSFSSKKALTEHQRTHTGEKPYKCPECGKSFSQRANLRAHQRTHTGKKTS | |
| - | cacagagagag | 482 | LEPGEKPYKCPECGKSFSQRANLRAHQRTHTGEKPYKCPECGKSFSQRANLRAHQRTHTGEKPYKCPEC | 964 |
| | agaaaaaaaa | | GKSFSQSSNLVRHQRTHTGEKPYKCPECGKSFSQLAHLRAHQRTHTGEKPYKCPECGKSFSRSDNLVRHQ | |
| | | | RTHTGEKPYKCPECGKSFSQLAHLRAHQRTHTGEKPYKCPECGKSFSSKKALTEHQRTHTGKKTS | |
| - | agagagagaga | 483 | LEPGEKPYKCPECGKSFSDSGNLRVHQRTHTGEKPYKCPECGKSFSQRANLRAHQRTHTGEKPYKCPECG | 965 |
| | aaaaaaaaac | | KSFSQRANLRAHQRTHTGEKPYKCPECGKSFSQSSNLVRHQRTHTGEKPYKCPECGKSFSQLAHLRAHQR | |
| | | | THTGEKPYKCPECGKSFSRSDNLVRHQRTHTGEKPYKCPECGKSFSQLAHLRAHQRTHTGKKTS | |
| - | gagagagaaaa | 484 | LEPGEKPYKCPECGKSFSRRDELNVHQRTHTHPRAPIPKPFQYKCPECGKSFSDSGNLRVHQRTHTGEKPY | 966 |
| | aaaaaacTatg | | KCPECGKSFSQRANLRAHQRTHTGEKPYKCPECGKSFSQRANLRAHQRTHTGEKPYKCPECGKSFSQSSN | |
| | | | LVRHQRTHTGEKPYKCPECGKSFSQLAHLRAHQRTHTGEKPYKCPECGKSFSRSDNLVRHQRTHTGKKT | |
| | | | S | |
| - | agagaaaaaaa | 485 | LEPGEKPYKCPECGKSFSQLAHLRAHQRTHTGEKPYKCPECGKSFSRRDELNVHQRTHTHPRAPIPKPFQY | 967 |
| | aaacTatgaga | | KCPECGKSFSDSGNLRVHQRTHTGEKPYKCPECGKSFSQRANLRAHQRTHTGEKPYKCPECGKSFSQRA | |
| | | | NLRAHQRTHTGEKPYKCPECGKSFSQSSNLVRHQRTHTGEKPYKCPECGKSFSQLAHLRAHQRTHTGKK | |
| | | | TS | |
| - | gaaaaaaaaaa | 486 | LEPGEKPYKCPECGKSFSDKKDLTRHQRTHTGEKPYKCPECGKSFSQLAHLRAHQRTHTGEKPYKCPECG | 968 |
| | cTatgagaacc | | KSFSRRDELNVHQRTHTHPRAPIPKPFQYKCPECGKSFSDSGNLRVHQRTHTGEKPYKCPECGKSFSQRA | |
| | | | NLRAHQRTHTGEKPYKCPECGKSFSQRANLRAHQRTHTGEKPYKCPECGKSFSQSSNLVRHQRTHTGKK | |
| | | | TS | |
| - | aaaaaaaacTa | 487 | LEPGEKPYKCPECGKSFSSKKHLAEHQRTHTGEKPYKCPECGKSFSDKKDLTRHQRTHTGEKPYKCPECG | 969 |
| | tgagaaccccc | | KSFSQLAHLRAHQRTHTGEKPYKCPECGKSFSRRDELNVHQRTHTHPRAPIPKPFQYKCPECGKSFSDSGN | |
| | | | LRVHQRTHTGEKPYKCPECGKSFSQRANLRAHQRTHTGEKPYKCPECGKSFSQRANLRAHQRTHTGKKT | |
| | | | S | |
| - | aaaaacTatga | 488 | LEPGEKPYKCPECGKSFSSKKHLAEHQRTHTGEKPYKCPECGKSFSSKKHLAEHQRTHTGEKPYKCPECG | 970 |
| | gaacccccccc | | KSFSDKKDLTRHQRTHTGEKPYKCPECGKSFSQLAHLRAHQRTHTGEKPYKCPECGKSFSRRDELNVHQ | |
| | | | RTHTHPRAPIPKPFQYKCPECGKSFSDSGNLRVHQRTHTGEKPYKCPECGKSFSQRANLRAHQRTHTGKK | |
| | | | TS | |
| - | aacTatgagaa | 489 | LEPGEKPYKCPECGKSFSSKKALTEHQRTHTGEKPYKCPECGKSFSSKKHLAEHQRTHTGEKPYKCPECG | 971 |
| | cccccccccac | | KSFSSKKHLAEHQRTHTGEKPYKCPECGKSFSDKKDLTRHQRTHTGEKPYKCPECGKSFSQLAHLRAHQ | |
| | | | RTHTGEKPYKCPECGKSFSRRDELNVHQRTHTHPRAPIPKPFQYKCPECGKSFSDSGNLRVHQRTHTGKK | |
| | | | TS | |
| - | ataATcaagaa | 490 | LEPGEKPYKCPECGKSFSSKKALTEHQRTHTGEKPYKCPECGKSFSQRANLRAHQRTHTGEKPYKCPECG | 972 |
| | aaggagaaaca | | KSFSRSDNLVRHQRTHTGEKPYKCPECGKSFSRKDNLKNHQRTHTGEKPYKCPECGKSFSQSSNLVRHQR | |
| | c | | THTGEKPYKCPECGKSFSQSGNLTEHQRTHTPNPHRRTDPSHKPFQYKCPECGKSFSQKSSLIAHQRTHTG | |
| | | | KKTS | |
| - | aagaaaaggag | 491 | LEPGEKPYKCPECGKSFSRSDNLVRHQRTHTGEKPYKCPECGKSFSSPADLTRHQRTHTGEKPYKCPECG | 973 |
| | aaacacagag | | KSFSDSGNLRVHQRTHTGEKPYKCPECGKSFSQLAHLRAHQRTHTGEKPYKCPECGKSFSRSDHLTNHQR | |
| | | | THTGEKPYKCPECGKSFSQRANLRAHQRTHTGEKPYKCPECGKSFSRKDNLKNHQRTHTGKKTS | |
| - | aaaaggagaaa | 492 | LEPGEKPYKCPECGKSFSQLAHLRAHQRTHTGEKPYKCPECGKSFSRSDNLVRHQRTHTGEKPYKCPECG | 974 |
| | cacagagaga | | KSFSSPADLTRHQRTHTGEKPYKCPECGKSFSDSGNLRVHQRTHTGEKPYKCPECGKSFSQLAHLRAHQR | |
| | | | THTGEKPYKCPECGKSFSRSDHLTNHQRTHTGEKPYKCPECGKSFSQRANLRAHQRTHTGKKTS | |
| - | aggagaaacac | 493 | LEPGEKPYKCPECGKSFSRSDNLVRHQRTHTGEKPYKCPECGKSFSQLAHLRAHQRTHTGEKPYKCPECG | 975 |
| | agagagagag | | KSFSRSDNLVRHQRTHTGEKPYKCPECGKSFSSPADLTRHQRTHTGEKPYKCPECGKSFSDSGNLRVHQR | |
| | | | THTGEKPYKCPECGKSFSQLAHLRAHQRTHTGEKPYKCPECGKSFSRSDHLTNHQRTHTGKKTS | |
| - | agaaacacaga | 494 | LEPGEKPYKCPECGKSFSQRANLRAHQRTHTGEKPYKCPECGKSFSRSDNLVRHQRTHTGEKPYKCPECG | 976 |
| | gagagagaaa | | KSFSQLAHLRAHQRTHTGEKPYKCPECGKSFSRSDNLVRHQRTHTGEKPYKCPECGKSFSSPADLTRHQR | |
| | | | THTGEKPYKCPECGKSFSDSGNLRVHQRTHTGEKPYKCPECGKSFSQLAHLRAHQRTHTGKKTS | |
| - | aacacagagag | 495 | LEPGEKPYKCPECGKSFSQRANLRAHQRTHTGEKPYKCPECGKSFSQRANLRAHQRTHTGEKPYKCPEC | 977 |
| | agagaaaaaa | | GKSFSRSDNLVRHQRTHTGEKPYKCPECGKSFSQLAHLRAHQRTHTGEKPYKCPECGKSFSRSDNLVRHQ | |
| | | | RTHTGEKPYKCPECGKSFSSPADLTRHQRTHTGEKPYKCPECGKSFSDSGNLRVHQRTHTGKKTS | |
| - | acagagagaga | 496 | LEPGEKPYKCPECGKSFSQRANLRAHQRTHTGEKPYKCPECGKSFSQRANLRAHQRTHTGEKPYKCPEC | 978 |
| | gaaaaaaaaa | | GKSFSQRANLRAHQRTHTGEKPYKCPECGKSFSRSDNLVRHQRTHTGEKPYKCPECGKSFSQLAHLRAH | |
| | | | QRTHTGEKPYKCPECGKSFSRSDNLVRHQRTHTGEKPYKCPECGKSFSSPADLTRHQRTHTGKKTS | |
| - | gagagagagaa | 497 | LEPGEKPYKCPECGKSFSTHLDLIRHQRTHTGEKPYKCPECGKSFSQRANLRAHQRTHTGEKPYKCPECG | 979 |
| | aaaaaaaact | | KSFSQRANLRAHQRTHTGEKPYKCPECGKSFSQRANLRAHQRTHTGEKPYKCPECGKSFSRSDNLVRHQ | |
| | | | RTHTGEKPYKCPECGKSFSQLAHLRAHQRTHTGEKPYKCPECGKSFSRSDNLVRHQRTHTGKKTS | |
| - | agagagaaaaa | 498 | LEPGEKPYKCPECGKSFSRRDELNVHQRTHTGEKPYKCPECGKSFSTHLDLIRHQRTHTGEKPYKCPECG | 980 |
| | aaaaactatg | | KSFSQRANLRAHQRTHTGEKPYKCPECGKSFSQRANLRAHQRTHTGEKPYKCPECGKSFSQRANLRAHQ | |
| | | | RTHTGEKPYKCPECGKSFSRSDNLVRHQRTHTGEKPYKCPECGKSFSQLAHLRAHQRTHTGKKTS | |
| - | gagaaaaaaaa | 499 | LEPGEKPYKCPECGKSFSQLAHLRAHQRTHTGEKPYKCPECGKSFSRRDELNVHQRTHTGEKPYKCPECG | 981 |
| | aactatgaga | | KSFSTHLDLIRHQRTHTGEKPYKCPECGKSFSQRANLRAHQRTHTGEKPYKCPECGKSFSQRANLRAHQR | |
| | | | THTGEKPYKCPECGKSFSQRANLRAHQRTHTGEKPYKCPECGKSFSRSDNLVRHQRTHTGKKTS | |
| - | aaaaaaaaaact | 500 | LEPGEKPYKCPECGKSFSDKKDLTRHQRTHTGEKPYKCPECGKSFSQLAHLRAHQRTHTGEKPYKCPECG | 982 |
| | atgagaacc | | KSFSRRDELNVHQRTHTGEKPYKCPECGKSFSTHLDLIRHQRTHTGEKPYKCPECGKSFSQRANLRAHQR | |
| | | | THTGEKPYKCPECGKSFSQRANLRAHQRTHTGEKPYKCPECGKSFSQRANLRAHQRTHTGKKTS | |
| - | aaaaaaactatg | 501 | LEPGEKPYKCPECGKSFSSKKHLAEHQRTHTGEKPYKCPECGKSFSDKKDLTRHQRTHTGEKPYKCPECG | 983 |
| | agaaccccc | | KSFSQLAHLRAHQRTHTGEKPYKCPECGKSFSRRDELNVHQRTHTGEKPYKCPECGKSFSTHLDLIRHQR | |
| | | | THTGEKPYKCPECGKSFSQRANLRAHQRTHTGEKPYKCPECGKSFSQRANLRAHQRTHTGKKTS | |
| - | aaaactatgaga | 502 | LEPGEKPYKCPECGKSFSSKKHLAEHQRTHTGEKPYKCPECGKSFSSKKHLAEHQRTHTGEKPYKCPECG | 984 |
| | acccccccc | | KSFSDKKDLTRHQRTHTGEKPYKCPECGKSFSQLAHLRAHQRTHTGEKPYKCPECGKSFSRRDELNVHQ | |
| | | | RTHTGEKPYKCPECGKSFSTHLDLIRHQRTHTGEKPYKCPECGKSFSQRANLRAHQRTHTGKKTS | |
| - | actatgagaacc | 503 | LEPGEKPYKCPECGKSFSSKKALTEHQRTHTGEKPYKCPECGKSFSSKKHLAEHQRTHTGEKPYKCPECG | 985 |
| | cccccccac | | KSFSSKKHLAEHQRTHTGEKPYKCPECGKSFSDKKDLTRHQRTHTGEKPYKCPECGKSFSQLAHLRAHQ | |
| | | | RTHTGEKPYKCPECGKSFSRRDELNVHQRTHTGEKPYKCPECGKSFSTHLDLIRHQRTHTGKKTS | |
| - | atgagaacccc | 504 | LEPGEKPYKCPECGKSFSSKKHLAEHQRTHTGEKPYKCPECGKSFSSKKALTEHQRTHTGEKPYKCPECG | 986 |
| | cccccacccc | | KSFSSKKHLAEHQRTHTGEKPYKCPECGKSFSSKKHLAEHQRTHTGEKPYKCPECGKSFSDKKDLTRHQR | |
| | | | THTGEKPYKCPECGKSFSQLAHLRAHQRTHTGEKPYKCPECGKSFSRRDELNVHQRTHTGKKTS | |
| - | agaaccccccc | 505 | LEPGEKPYKCPECGKSFSRSDELVRHQRTHTGEKPYKCPECGKSFSSKKHLAEHQRTHTGEKPYKCPECG | 987 |
| | ccaccccgtg | | KSFSSKKALTEHQRTHTGEKPYKCPECGKSFSSKKHLAEHQRTHTGEKPYKCPECGKSFSSKKHLAEHQR | |
| | | | THTGEKPYKCPECGKSFSDKKDLTRHQRTHTGEKPYKCPECGKSFSQLAHLRAHQRTHTGKKTS | |
| - | accccccccca | 506 | LEPGEKPYKCPECGKSFSHKNALQNHQRTHTGEKPYKCPECGKSFSRSDELVRHQRTHTGEKPYKCPECG | 988 |
| | ccccgtgatt | | KSFSSKKHLAEHQRTHTGEKPYKCPECGKSFSSKKALTEHQRTHTGEKPYKCPECGKSFSSKKHLAEHQR | |
| | | | THTGEKPYKCPECGKSFSSKKHLAEHQRTHTGEKPYKCPECGKSFSDKKDLTRHQRTHTGKKTS | |
| - | cccccccaccc | 507 | LEPGEKPYKCPECGKSFSRADNLTEHQRTHTPNPHRRTDPSHKPFQYKCPECGKSFSHKNALQNHQRTHT | 989 |
| | cgtgattATca | | GEKPYKCPECGKSFSRSDELVRHQRTHTGEKPYKCPECGKSFSSKKHLAEHQRTHTGEKPYKCPECGKSF | |
| | g | | SSKKALTEHQRTHTGEKPYKCPECGKSFSSKKHLAEHQRTHTGEKPYKCPECGKSFSSKKHLAEHQRTHT | |
| | | | GKKTS | |
| - | ccccaccccgt | 508 | LEPGEKPYKCPECGKSFSHTGHLLEHQRTHTGEKPYKCPECGKSFSRADNLTEHQRTHTPNPHRRTDPSH | 990 |
| | gattATcagcg | | KPFQYKCPECGKSFSHKNALQNHQRTHTGEKPYKCPECGKSFSRSDELVRHQRTHTGEKPYKCPECGKSF | |
| | c | | SSKKHLAEHQRTHTGEKPYKCPECGKSFSSKKALTEHQRTHTGEKPYKCPECGKSFSSKKHLAEHQRTHT | |
| | | | GKKTS | |
| - | caccccgtgatt | 509 | LEPGEKPYKCPECGKSFSSPADLTRHQRTHTGEKPYKCPECGKSFSHTGHLLEHQRTHTGEKPYKCPECG | 991 |
| | ATcagcgcac | | KSFSRADNLTEHQRTHTPNPHRRTDPSHKPFQYKCPECGKSFSHKNALQNHQRTHTGEKPYKCPECGKSF | |
| | a | | SRSDELVRHQRTHTGEKPYKCPECGKSFSSKKHLAEHQRTHTGEKPYKCPECGKSFSSKKALTEHQRTHT | |
| | | | GKKTS | |
| - | cccgtgattAT | 510 | LEPGEKPYKCPECGKSFSSKKALTEHQRTHTGEKPYKCPECGKSFSSPADLTRHQRTHTGEKPYKCPECG | 992 |
| | cagcgcacaca | | KSFSHTGHLLEHQRTHTGEKPYKCPECGKSFSRADNLTEHQRTHTPNPHRRTDPSHKPFQYKCPECGKSFS | |
| | C | | HKNALQNHQRTHTGEKPYKCPECGKSFSRSDELVRHQRTHTGEKPYKCPECGKSFSSKKHLAEHQRTHT | |
| | | | GKKTS | |
| - | gtgattATcag | 511 | LEPGEKPYKCPECGKSFSTSGNLTEHQRTHTHPRAPIPKPFQYKCPECGKSFSSKKALTEHQRTHTGEKPY | 993 |
| | cgcacacacTc | | KCPECGKSFSSPADLTRHQRTHTGEKPYKCPECGKSFSHTGHLLEHQRTHTGEKPYKCPECGKSFSRADN | |
| | at | | LTEHQRTHTPNPHRRTDPSHKPFQYKCPECGKSFSHKNALQNHQRTHTGEKPYKCPECGKSFSRSDELVR | |
| | | | HQRTHTGKKTS | |
| - | attATcagcgc | 512 | LEPGEKPYKCPECGKSFSQSGHLTEHQRTHTGEKPYKCPECGKSFSTSGNLTEHQRTHTHPRAPIPKPFQY | 994 |
| | acacacTcatc | | KCPECGKSFSSKKALTEHQRTHTGEKPYKCPECGKSFSSPADLTRHQRTHTGEKPYKCPECGKSFSHTGH | |
| | ga | | LLEHQRTHTGEKPYKCPECGKSFSRADNLTEHQRTHTPNPHRRTDPSHKPFQYKCPECGKSFSHKNALQN | |
| | | | HQRTHTGKKTS | |
| - | agaaaaggaga | 513 | LEPGEKPYKCPECGKSFSQLAHLRAHQRTHTGEKPYKCPECGKSFSRADNLTEHQRTHTGEKPYKCPECG | 995 |
| | aacacagaga | | KSFSSPADLTRHQRTHTGEKPYKCPECGKSFSQSSNLVRHQRTHTGEKPYKCPECGKSFSQRAHLERHQR | |
| | | | THTGEKPYKCPECGKSFSQRANLRAHQRTHTGEKPYKCPECGKSFSQLAHLRAHQRTHTGKKTS | |
| - | aaaggagaaac | 514 | LEPGEKPYKCPECGKSFSRSDNLVRHQRTHTGEKPYKCPECGKSFSQLAHLRAHQRTHTGEKPYKCPECG | 996 |
| | acagagagag | | KSFSRADNLTEHQRTHTGEKPYKCPECGKSFSSPADLTRHQRTHTGEKPYKCPECGKSFSQSSNLVRHQR | |
| | | | THTGEKPYKCPECGKSFSQRAHLERHQRTHTGEKPYKCPECGKSFSQRANLRAHQRTHTGKKTS | |
| - | ggagaaacaca | 515 | LEPGEKPYKCPECGKSFSQLAHLRAHQRTHTGEKPYKCPECGKSFSRSDNLVRHQRTHTGEKPYKCPECG | 997 |
| | gagagagaga | | KSFSQLAHLRAHQRTHTGEKPYKCPECGKSFSRADNLTEHQRTHTGEKPYKCPECGKSFSSPADLTRHQR | |
| | | | THTGEKPYKCPECGKSFSQSSNLVRHQRTHTGEKPYKCPECGKSFSQRAHLERHQRTHTGKKTS | |
| - | gaaacacagag | 516 | LEPGEKPYKCPECGKSFSQRANLRAHQRTHTGEKPYKCPECGKSFSQLAHLRAHQRTHTGEKPYKCPECG | 998 |
| | agagagaaaa | | KSFSRSDNLVRHQRTHTGEKPYKCPECGKSFSQLAHLRAHQRTHTGEKPYKCPECGKSFSRADNLTEHQR | |
| | | | THTGEKPYKCPECGKSFSSPADLTRHQRTHTGEKPYKCPECGKSFSQSSNLVRHQRTHTGKKTS | |
| - | acacagagaga | 517 | LEPGEKPYKCPECGKSFSQRANLRAHQRTHTGEKPYKCPECGKSFSQRANLRAHQRTHTGEKPYKCPEC | 999 |
| | gagaaaaaaa | | GKSFSQLAHLRAHQRTHTGEKPYKCPECGKSFSRSDNLVRHQRTHTGEKPYKCPECGKSFSQLAHLRAH | |
| | | | QRTHTGEKPYKCPECGKSFSRADNLTEHQRTHTGEKPYKCPECGKSFSSPADLTRHQRTHTGKKTS | |
| - | cagagagagag | 518 | LEPGEKPYKCPECGKSFSQRANLRAHQRTHTGEKPYKCPECGKSFSQRANLRAHQRTHTGEKPYKCPEC | 1000 |
| | aaaaaaaaaa | | GKSFSQRANLRAHQRTHTGEKPYKCPECGKSFSQLAHLRAHQRTHTGEKPYKCPECGKSFSRSDNLVRH | |
| | | | QRTHTGEKPYKCPECGKSFSQLAHLRAHQRTHTGEKPYKCPECGKSFSRADNLTEHQRTHTGKKTS | |
| - | agagagagaaa | 519 | LEPGEKPYKCPECGKSFSQNSTLTEHQRTHTGEKPYKCPECGKSFSQRANLRAHQRTHTGEKPYKCPECG | 1001 |
| | aaaaaaacta | | KSFSQRANLRAHQRTHTGEKPYKCPECGKSFSQRANLRAHQRTHTGEKPYKCPECGKSFSQLAHLRAHQ | |
| | | | RTHTGEKPYKCPECGKSFSRSDNLVRHQRTHTGEKPYKCPECGKSFSQLAHLRAHQRTHTGKKTS | |
| - | gagagaaaaaa | 520 | LEPGEKPYKCPECGKSFSQAGHLASHQRTHTGEKPYKCPECGKSFSQNSTLTEHQRTHTGEKPYKCPECG | 1002 |
| | aaaactatga | | KSFSQRANLRAHQRTHTGEKPYKCPECGKSFSQRANLRAHQRTHTGEKPYKCPECGKSFSQRANLRAHQ | |
| | | | RTHTGEKPYKCPECGKSFSQLAHLRAHQRTHTGEKPYKCPECGKSFSRSDNLVRHQRTHTGKKTS | |
| - | agaaaaaaaaa | 521 | LEPGEKPYKCPECGKSFSQSSNLVRHQRTHTGEKPYKCPECGKSFSQAGHLASHQRTHTGEKPYKCPECG | 1003 |
| | actatgagaa | | KSFSQNSTLTEHQRTHTGEKPYKCPECGKSFSQRANLRAHQRTHTGEKPYKCPECGKSFSQRANLRAHQR | |
| | | | THTGEKPYKCPECGKSFSQRANLRAHQRTHTGEKPYKCPECGKSFSQLAHLRAHQRTHTGKKTS | |
| - | aaaaaaaaacta | 522 | LEPGEKPYKCPECGKSFSSKKHLAEHQRTHTGEKPYKCPECGKSFSQSSNLVRHQRTHTGEKPYKCPECG | 1004 |
| | tgagaaccc | | KSFSQAGHLASHQRTHTGEKPYKCPECGKSFSQNSTLTEHQRTHTGEKPYKCPECGKSFSQRANLRAHQR | |
| | | | THTGEKPYKCPECGKSFSQRANLRAHQRTHTGEKPYKCPECGKSFSQRANLRAHQRTHTGKKTS | |
| - | aaaaaactatga | 523 | LEPGEKPYKCPECGKSFSSKKHLAEHQRTHTGEKPYKCPECGKSFSSKKHLAEHQRTHTGEKPYKCPECG | 1005 |
| | gaacccccc | | KSFSQSSNLVRHQRTHTGEKPYKCPECGKSFSQAGHLASHQRTHTGEKPYKCPECGKSFSQNSTLTEHQR | |
| | | | THTGEKPYKCPECGKSFSQRANLRAHQRTHTGEKPYKCPECGKSFSQRANLRAHQRTHTGKKTS | |
| - | aaactatgagaa | 524 | LEPGEKPYKCPECGKSFSSKKHLAEHQRTHTGEKPYKCPECGKSFSSKKHLAEHQRTHTGEKPYKCPECG | 1006 |
| | CCCCCCCCC | | KSFSSKKHLAEHQRTHTGEKPYKCPECGKSFSQSSNLVRHQRTHTGEKPYKCPECGKSFSQAGHLASHQR | |
| | | | THTGEKPYKCPECGKSFSQNSTLTEHQRTHTGEKPYKCPECGKSFSQRANLRAHQRTHTGKKTS | |
| - | ctatgagaaccc | 525 | LEPGEKPYKCPECGKSFSDKKDLTRHQRTHTGEKPYKCPECGKSFSSKKHLAEHQRTHTGEKPYKCPECG | 1007 |
| | ccccccacc | | KSFSSKKHLAEHQRTHTGEKPYKCPECGKSFSSKKHLAEHQRTHTGEKPYKCPECGKSFSQSSNLVRHQR | |
| | | | THTGEKPYKCPECGKSFSQAGHLASHQRTHTGEKPYKCPECGKSFSQNSTLTEHQRTHTGKKTS | |
| - | tgagaaccccc | 526 | LEPGEKPYKCPECGKSFSRNDTLTEHQRTHTGEKPYKCPECGKSFSDKKDLTRHQRTHTGEKPYKCPECG | 1008 |
| | ccccaccccg | | KSFSSKKHLAEHQRTHTGEKPYKCPECGKSFSSKKHLAEHQRTHTGEKPYKCPECGKSFSSKKHLAEHQR | |
| | | | THTGEKPYKCPECGKSFSQSSNLVRHQRTHTGEKPYKCPECGKSFSQAGHLASHQRTHTGKKTS | |
| - | gaacccccccc | 527 | LEPGEKPYKCPECGKSFSQAGHLASHQRTHTGEKPYKCPECGKSFSRNDTLTEHQRTHTGEKPYKCPECG | 1009 |
| | caccccgtga | | KSFSDKKDLTRHQRTHTGEKPYKCPECGKSFSSKKHLAEHQRTHTGEKPYKCPECGKSFSSKKHLAEHQR | |
| | | | THTGEKPYKCPECGKSFSSKKHLAEHQRTHTGEKPYKCPECGKSFSQSSNLVRHQRTHTGKKTS | |
| - | gagaacccccc | 528 | LEPGEKPYKCPECGKSFSSRRTCRAHQRTHTGEKPYKCPECGKSFSSKKHLAEHQRTHTGEKPYKCPECG | 1010 |
| | cccaccccgt | | KSFSTSHSLTEHQRTHTGEKPYKCPECGKSFSSKKHLAEHQRTHTGEKPYKCPECGKSFSSKKHLAEHQR | |
| | | | THTGEKPYKCPECGKSFSDSGNLRVHQRTHTGEKPYKCPECGKSFSRSDNLVRHQRTHTGKKTS | |
| - | aaccccccccc | 529 | LEPGEKPYKCPECGKSFSTSGNLVRHQRTHTGEKPYKCPECGKSFSSRRTCRAHQRTHTGEKPYKCPECG | 1011 |
| | accccgtgat | | KSFSSKKHLAEHQRTHTGEKPYKCPECGKSFSTSHSLTEHQRTHTGEKPYKCPECGKSFSSKKHLAEHQR | |
| | | | THTGEKPYKCPECGKSFSSKKHLAEHQRTHTGEKPYKCPECGKSFSDSGNLRVHQRTHTGKKTS | |
| - | cagcgcacaca | 530 | LEPGEKPYKCPECGKSFSQRANLRAHQRTHTGEKPYKCPECGKSFSQSGHLTEHQRTHTGEKPYKCPECG | 1012 |
| | cTcatcgaaaa | | KSFSTSGNLTEHQRTHTHPRAPIPKPFQYKCPECGKSFSSKKALTEHQRTHTGEKPYKCPECGKSFSSPADL | |
| | | | TRHQRTHTGEKPYKCPECGKSFSHTGHLLEHQRTHTGEKPYKCPECGKSFSRADNLTEHQRTHTGKKTS | |
| - | cgcacacacTc | 531 | LEPGEKPYKCPECGKSFSQRANLRAHQRTHTGEKPYKCPECGKSFSQRANLRAHQRTHTGEKPYKCPEC | 1013 |
| | atcgaaaaaaa | | GKSFSQSGHLTEHQRTHTGEKPYKCPECGKSFSTSGNLTEHQRTHTHPRAPIPKPFQYKCPECGKSFSSKK | |
| | | | ALTEHQRTHTGEKPYKCPECGKSFSSPADLTRHQRTHTGEKPYKCPECGKSFSHTGHLLEHQRTHTGKKT | |
| | | | S | |
| - | acacacTcatc | 532 | LEPGEKPYKCPECGKSFSRSDHLTTHQRTHTPNPHRRTDPSHKPFQYKCPECGKSFSQRANLRAHQRTHT | 1014 |
| | gaaaaaaaTTt | | GEKPYKCPECGKSFSQRANLRAHQRTHTGEKPYKCPECGKSFSQSGHLTEHQRTHTGEKPYKCPECGKSF | |
| | gg | | STSGNLTEHQRTHTHPRAPIPKPFQYKCPECGKSFSSKKALTEHQRTHTGEKPYKCPECGKSFSSPADLTR | |
| | | | HQRTHTGKKTS | |
| - | cacTcatcgaa | 533 | LEPGEKPYKCPECGKSFSHKNALQNHQRTHTGEKPYKCPECGKSFSRSDHLTTHQRTHTPNPHRRTDPSH | 1015 |
| | aaaaaTTtgga | | KPFQYKCPECGKSFSQRANLRAHQRTHTGEKPYKCPECGKSFSQRANLRAHQRTHTGEKPYKCPECGKS | |
| | tt | | FSQSGHLTEHQRTHTGEKPYKCPECGKSFSTSGNLTEHQRTHTHPRAPIPKPFQYKCPECGKSFSSKKALT | |
| | | | EHQRTHTGKKTS | |
| - | agcgcacacac | 534 | LEPGEKPYKCPECGKSFSQRANLRAHQRTHTGEKPYKCPECGKSFSQSGHLTEHQRTHTGEKPYKCPECG | 1016 |
| | tcatcgaaaa | | KSFSTSGNLTEHQRTHTGEKPYKCPECGKSFSTHLDLIRHQRTHTGEKPYKCPECGKSFSSKKALTEHQRT | |
| | | | HTGEKPYKCPECGKSFSQSGDLRRHQRTHTGEKPYKCPECGKSFSERSHLREHQRTHTGKKTS | |
| - | gcacacactcat | 535 | LEPGEKPYKCPECGKSFSQRANLRAHQRTHTGEKPYKCPECGKSFSQRANLRAHQRTHTGEKPYKCPEC | 1017 |
| | cgaaaaaaa | | GKSFSQSGHLTEHQRTHTGEKPYKCPECGKSFSTSGNLTEHQRTHTGEKPYKCPECGKSFSTHLDLIRHQR | |
| | | | THTGEKPYKCPECGKSFSSKKALTEHQRTHTGEKPYKCPECGKSFSQSGDLRRHQRTHTGKKTS | |
| - | cacactcatcga | 536 | LEPGEKPYKCPECGKSFSRSDHLTTHQRTHTPNPHRRTDPSHKPFQYKCPECGKSFSQRANLRAHQRTHT | 1018 |
| | aaaaaaTTtgg | | GEKPYKCPECGKSFSQRANLRAHQRTHTGEKPYKCPECGKSFSQSGHLTEHQRTHTGEKPYKCPECGKSF | |
| | | | STSGNLTEHQRTHTGEKPYKCPECGKSFSTHLDLIRHQRTHTGEKPYKCPECGKSFSSKKALTEHQRTHTG | |
| | | | KKTS | |
| - | actcatcgaaaa | 537 | LEPGEKPYKCPECGKSFSHKNALQNHQRTHTGEKPYKCPECGKSFSRSDHLTTHQRTHTPNPHRRTDPSH | 1019 |
| | aaaTTtggatt | | KPFQYKCPECGKSFSQRANLRAHQRTHTGEKPYKCPECGKSFSQRANLRAHQRTHTGEKPYKCPECGKS | |
| | | | FSQSGHLTEHQRTHTGEKPYKCPECGKSFSTSGNLTEHQRTHTGEKPYKCPECGKSFSTHLDLIRHQRTHT | |
| | | | GKKTS | |
| - | catcgaaaaaaa | 538 | LEPGEKPYKCPECGKSFSHKNALQNHQRTHTGEKPYKCPECGKSFSHKNALQNHQRTHTGEKPYKCPEC | 1020 |
| | TTtggattatt | | GKSFSRSDHLTTHQRTHTPNPHRRTDPSHKPFQYKCPECGKSFSQRANLRAHQRTHTGEKPYKCPECGKS | |
| | | | FSQRANLRAHQRTHTGEKPYKCPECGKSFSQSGHLTEHQRTHTGEKPYKCPECGKSFSTSGNLTEHQRTH | |
| | | | TGKKTS | |
| - | cgaaaaaaaT | 539 | LEPGEKPYKCPECGKSFSQLAHLRAHQRTHTGEKPYKCPECGKSFSHKNALQNHQRTHTGEKPYKCPEC | 1021 |
| | Ttggattattag | | GKSFSHKNALQNHQRTHTGEKPYKCPECGKSFSRSDHLTTHQRTHTPNPHRRTDPSHKPFQYKCPECGKS | |
| | a | | FSQRANLRAHQRTHTGEKPYKCPECGKSFSQRANLRAHQRTHTGEKPYKCPECGKSFSQSGHLTEHQRT | |
| | | | HTGKKTS | |
| - | aaaaaaTTtgg | 540 | LEPGEKPYKCPECGKSFSQLAHLRAHQRTHTGEKPYKCPECGKSFSQLAHLRAHQRTHTGEKPYKCPECG | 1022 |
| | attattagaaga | | KSFSHKNALQNHQRTHTGEKPYKCPECGKSFSHKNALQNHQRTHTGEKPYKCPECGKSFSRSDHLTTHQ | |
| | | | RTHTPNPHRRTDPSHKPFQYKCPECGKSFSQRANLRAHQRTHTGEKPYKCPECGKSFSQRANLRAHQRTH | |
| | | | TGKKTS | |
| - | aaaTTtggatt | 541 | LEPGEKPYKCPECGKSFSRSDNLVRHQRTHTGEKPYKCPECGKSFSQLAHLRAHQRTHTGEKPYKCPECG | 1023 |
| | attagaagagag | | KSFSQLAHLRAHQRTHTGEKPYKCPECGKSFSHKNALQNHQRTHTGEKPYKCPECGKSFSHKNALQNHQ | |
| | | | RTHTGEKPYKCPECGKSFSRSDHLTTHQRTHTPNPHRRTDPSHKPFQYKCPECGKSFSQRANLRAHQRTH | |
| | | | TGKKTS | |
| - | gcgcacacaC | 542 | LEPGEKPYKCPECGKSFSQRANLRAHQRTHTGEKPYKCPECGKSFSQRANLRAHQRTHTGEKPYKCPEC | 1024 |
| | Tcatcgaaaaa | | GKSFSQSGHLTEHQRTHTGEKPYKCPECGKSFSTSGNLTEHQRTHTPNPHRRTDPSHKPFQYKCPECGKSF | |
| | aa | | SSPADLTRHQRTHTGEKPYKCPECGKSFSSKKALTEHQRTHTGEKPYKCPECGKSFSRSDDLVRHQRTHT | |
| | | | GKKTS | |
| - | cacacaCTcat | 543 | LEPGEKPYKCPECGKSFSRSDHLTTHQRTHTPNPHRRTDPSHKPFQYKCPECGKSFSQRANLRAHQRTHT | 1025 |
| | cgaaaaaaaT | | GEKPYKCPECGKSFSQRANLRAHQRTHTGEKPYKCPECGKSFSQSGHLTEHQRTHTGEKPYKCPECGKSF | |
| | Ttgg | | STSGNLTEHQRTHTPNPHRRTDPSHKPFQYKCPECGKSFSSPADLTRHQRTHTGEKPYKCPECGKSFSSKK | |
| | | | ALTEHQRTHTGKKTS | |
| - | acaCTcatcga | 544 | LEPGEKPYKCPECGKSFSHKNALQNHQRTHTGEKPYKCPECGKSFSRSDHLTTHQRTHTPNPHRRTDPSH | 1026 |
| | aaaaaaTTtgg | | KPFQYKCPECGKSFSQRANLRAHQRTHTGEKPYKCPECGKSFSQRANLRAHQRTHTGEKPYKCPECGKS | |
| | att | | FSQSGHLTEHQRTHTGEKPYKCPECGKSFSTSGNLTEHQRTHTPNPHRRTDPSHKPFQYKCPECGKSFSSP | |
| | | | ADLTRHQRTHTGKKTS | |
| - | gaaaaaaatTt | 545 | LEPGEKPYKCPECGKSFSQLAHLRAHQRTHTGEKPYKCPECGKSFSHKNALQNHQRTHTGEKPYKCPEC | 1027 |
| | ggattattaga | | GKSFSHKNALQNHQRTHTGEKPYKCPECGKSFSRSDHLTTHQRTHTHPRAPIPKPFQYKCPECGKSFSTTG | |
| | | | NLTVHQRTHTGEKPYKCPECGKSFSQRANLRAHQRTHTGEKPYKCPECGKSFSQSSNLVRHQRTHTGKK | |
| | | | TS | |
| - | aaaaatTtggat | 546 | LEPGEKPYKCPECGKSFSQLAHLRAHQRTHTGEKPYKCPECGKSFSQLAHLRAHQRTHTGEKPYKCPECG | 1028 |
| | tattagaaga | | KSFSHKNALQNHQRTHTGEKPYKCPECGKSFSHKNALQNHQRTHTGEKPYKCPECGKSFSRSDHLTTHQ | |
| | | | RTHTHPRAPIPKPFQYKCPECGKSFSTTGNLTVHQRTHTGEKPYKCPECGKSFSQRANLRAHQRTHTGKK | |
| | | | TS | |
| - | aatTtggattatt | 547 | LEPGEKPYKCPECGKSFSRSDNLVRHQRTHTGEKPYKCPECGKSFSQLAHLRAHQRTHTGEKPYKCPECG | 1029 |
| | agaagagag | | KSFSQLAHLRAHQRTHTGEKPYKCPECGKSFSHKNALQNHQRTHTGEKPYKCPECGKSFSHKNALQNHQ | |
| | | | RTHTGEKPYKCPECGKSFSRSDHLTTHQRTHTHPRAPIPKPFQYKCPECGKSFSTTGNLTVHQRTHTGKKT | |
| | | | S | |
| - | aaaaaaatttgg | 548 | LEPGEKPYKCPECGKSFSQLAHLRAHQRTHTGEKPYKCPECGKSFSHKNALQNHQRTHTGEKPYKCPEC | 1030 |
| | attattaga | | GKSFSHKNALQNHQRTHTGEKPYKCPECGKSFSRSDHLTTHQRTHTGEKPYKCPECGKSFSHKNALQNH | |
| | | | QRTHTGEKPYKCPECGKSFSQRANLRAHQRTHTGEKPYKCPECGKSFSQRANLRAHQRTHTGKKTS | |
| - | aaaatttggatta | 549 | LEPGEKPYKCPECGKSFSQLAHLRAHQRTHTGEKPYKCPECGKSFSQLAHLRAHQRTHTGEKPYKCPECG | 1031 |
| | ttagaaga | | KSFSHKNALQNHQRTHTGEKPYKCPECGKSFSHKNALQNHQRTHTGEKPYKCPECGKSFSRSDHLTTHQ | |
| | | | RTHTGEKPYKCPECGKSFSHKNALQNHQRTHTGEKPYKCPECGKSFSQRANLRAHQRTHTGKKTS | |
| - | atttggattatta | 550 | LEPGEKPYKCPECGKSFSRSDNLVRHQRTHTGEKPYKCPECGKSFSQLAHLRAHQRTHTGEKPYKCPECG | 1032 |
| | gaagagag | | KSFSQLAHLRAHQRTHTGEKPYKCPECGKSFSHKNALQNHQRTHTGEKPYKCPECGKSFSHKNALQNHQ | |
| | | | RTHTGEKPYKCPECGKSFSRSDHLTTHQRTHTGEKPYKCPECGKSFSHKNALQNHQRTHTGKKTS | |
| - | tggattattagaa | 551 | LEPGEKPYKCPECGKSFSRSDHLTNHQRTHTGEKPYKCPECGKSFSRSDNLVRHQRTHTGEKPYKCPECG | 1033 |
| | gagagagg | | KSFSQLAHLRAHQRTHTGEKPYKCPECGKSFSQLAHLRAHQRTHTGEKPYKCPECGKSFSHKNALQNHQ | |
| | | | RTHTGEKPYKCPECGKSFSHKNALQNHQRTHTGEKPYKCPECGKSFSRSDHLTTHQRTHTGKKTS | |
| - | attattagaaga | 552 | LEPGEKPYKCPECGKSFSRNDALTEHQRTHTHPRAPIPKPFQYKCPECGKSFSRSDHLTNHQRTHTGEKPY | 1034 |
| | gagaggTctg | | KCPECGKSFSRSDNLVRHQRTHTGEKPYKCPECGKSFSQLAHLRAHQRTHTGEKPYKCPECGKSFSQLAH | |
| | | | LRAHQRTHTGEKPYKCPECGKSFSHKNALQNHQRTHTGEKPYKCPECGKSFSHKNALQNHQRTHTGKKT | |
| | | | S | |
| - | attagaagagag | 553 | LEPGEKPYKCPECGKSFSRSDKLTEHQRTHTGEKPYKCPECGKSFSRNDALTEHQRTHTHPRAPIPKPFQY | 1035 |
| | aggTctgcgg | | KCPECGKSFSRSDHLTNHQRTHTGEKPYKCPECGKSFSRSDNLVRHQRTHTGEKPYKCPECGKSFSQLAH | |
| | | | LRAHQRTHTGEKPYKCPECGKSFSQLAHLRAHQRTHTGEKPYKCPECGKSFSHKNALQNHQRTHTGKKT | |
| | | | S | |
| - | agaagagagag | 554 | LEPGEKPYKCPECGKSFSTTGALTEHQRTHTGEKPYKCPECGKSFSRSDKLTEHQRTHTGEKPYKCPECG | 1036 |
| | gTctgcggctt | | KSFSRNDALTEHQRTHTHPRAPIPKPFQYKCPECGKSFSRSDHLTNHQRTHTGEKPYKCPECGKSFSRSDN | |
| | | | LVRHQRTHTGEKPYKCPECGKSFSQLAHLRAHQRTHTGEKPYKCPECGKSFSQLAHLRAHQRTHTGKKT | |
| | | | S | |
| - | agagagaggT | 555 | LEPGEKPYKCPECGKSFSTSHSLTEHQRTHTGEKPYKCPECGKSFSTTGALTEHQRTHTGEKPYKCPECGK | 1037 |
| | ctgcggcttcca | | SFSRSDKLTEHQRTHTGEKPYKCPECGKSFSRNDALTEHQRTHTHPRAPIPKPFQYKCPECGKSFSRSDHL | |
| | | | TNHQRTHTGEKPYKCPECGKSFSRSDNLVRHQRTHTGEKPYKCPECGKSFSQLAHLRAHQRTHTGKKTS | |
| - | gagaggTctgc | 556 | LEPGEKPYKCPECGKSFSSKKALTEHQRTHTGEKPYKCPECGKSFSTSHSLTEHQRTHTGEKPYKCPECGK | 1038 |
| | ggcttccacac | | SFSTTGALTEHQRTHTGEKPYKCPECGKSFSRSDKLTEHQRTHTGEKPYKCPECGKSFSRNDALTEHQRT | |
| | | | HTHPRAPIPKPFQYKCPECGKSFSRSDHLTNHQRTHTGEKPYKCPECGKSFSRSDNLVRHQRTHTGKKTS | |
| - | aggTctgcggc | 557 | LEPGEKPYKCPECGKSFSSRRTCRAHQRTHTGEKPYKCPECGKSFSSKKALTEHQRTHTGEKPYKCPECG | 1039 |
| | ttccacaccgt | | KSFSTSHSLTEHQRTHTGEKPYKCPECGKSFSTTGALTEHQRTHTGEKPYKCPECGKSFSRSDKLTEHQRT | |
| | | | HTGEKPYKCPECGKSFSRNDALTEHQRTHTHPRAPIPKPFQYKCPECGKSFSRSDHLTNHQRTHTGKKTS | |
| - | ggaTTattaga | 558 | LEPGEKPYKCPECGKSFSRNDALTEHQRTHTHPRAPIPKPFQYKCPECGKSFSRSDHLTNHQRTHTGEKPY | 1040 |
| | agagagaggT | | KCPECGKSFSRSDNLVRHQRTHTGEKPYKCPECGKSFSQLAHLRAHQRTHTGEKPYKCPECGKSFSQLAH | |
| | ctg | | LRAHQRTHTGEKPYKCPECGKSFSHKNALQNHQRTHTPNPHRRTDPSHKPFQYKCPECGKSFSQRAHLER | |
| | | | HQRTHTGKKTS | |
| - | gatTattagaag | 559 | LEPGEKPYKCPECGKSFSRNDALTEHQRTHTHPRAPIPKPFQYKCPECGKSFSRSDHLTNHQRTHTGEKPY | 1041 |
| | agagaggTctg | | KCPECGKSFSRSDNLVRHQRTHTGEKPYKCPECGKSFSQLAHLRAHQRTHTGEKPYKCPECGKSFSQLAH | |
| | | | LRAHQRTHTGEKPYKCPECGKSFSHKNALQNHQRTHTHPRAPIPKPFQYKCPECGKSFSTSGNLVRHQRT | |
| | | | HTGKKTS | |
| - | tagaagagaga | 560 | LEPGEKPYKCPECGKSFSTSGELVRHQRTHTGEKPYKCPECGKSFSRSDDLVRHQRTHTHPRAPIPKPFQY | 1042 |
| | ggtcTgcggct | | KCPECGKSFSDPGALVRHQRTHTGEKPYKCPECGKSFSRSDNLVRHQRTHTGEKPYKCPECGKSFSQLAH | |
| | | | LRAHQRTHTGEKPYKCPECGKSFSRKDNLKNHQRTHTGEKPYKCPECGKSFSREDNLHTHQRTHTGKKT | |
| | | | S | |
| - | aagagagaggt | 561 | LEPGEKPYKCPECGKSFSTSHSLTEHQRTHTHPRAPIPKPFQYKCPECGKSFSTSGELVRHQRTHTGEKPY | 1043 |
| | cTgcggctTcc | | KCPECGKSFSRSDDLVRHQRTHTHPRAPIPKPFQYKCPECGKSFSDPGALVRHQRTHTGEKPYKCPECGKS | |
| | a | | FSRSDNLVRHQRTHTGEKPYKCPECGKSFSQLAHLRAHQRTHTGEKPYKCPECGKSFSRKDNLKNHQRTH | |
| | | | TGKKTS | |
| - | agagaggtcTg | 562 | LEPGEKPYKCPECGKSFSSKKALTEHQRTHTGEKPYKCPECGKSFSTSHSLTEHQRTHTHPRAPIPKPFQY | 1044 |
| | cggctTccaca | | KCPECGKSFSTSGELVRHQRTHTGEKPYKCPECGKSFSRSDDLVRHQRTHTHPRAPIPKPFQYKCPECGKS | |
| | c | | FSDPGALVRHQRTHTGEKPYKCPECGKSFSRSDNLVRHQRTHTGEKPYKCPECGKSFSQLAHLRAHQRT | |
| | | | HTGKKTS | |
| - | gaggtcTgcg | 563 | LEPGEKPYKCPECGKSFSSRRTCRAHQRTHTGEKPYKCPECGKSFSSKKALTEHQRTHTGEKPYKCPECG | 1045 |
| | gctTccacacc | | KSFSTSHSLTEHQRTHTHPRAPIPKPFQYKCPECGKSFSTSGELVRHQRTHTGEKPYKCPECGKSFSRSDD | |
| | gt | | LVRHQRTHTHPRAPIPKPFQYKCPECGKSFSDPGALVRHQRTHTGEKPYKCPECGKSFSRSDNLVRHQRTH | |
| | | | TGKKTS | |
| - | gtcTgcggctT | 564 | LEPGEKPYKCPECGKSFSSPADLTRHQRTHTGEKPYKCPECGKSFSSRRTCRAHQRTHTGEKPYKCPECG | 1046 |
| | ccacaccgtaca | | KSFSSKKALTEHQRTHTGEKPYKCPECGKSFSTSHSLTEHQRTHTHPRAPIPKPFQYKCPECGKSFSTSGE | |
| | | | LVRHQRTHTGEKPYKCPECGKSFSRSDDLVRHQRTHTHPRAPIPKPFQYKCPECGKSFSDPGALVRHQRTH | |
| | | | TGKKTS | |
| - | gaagagagagg | 565 | LEPGEKPYKCPECGKSFSTTGALTEHQRTHTGEKPYKCPECGKSFSRSDKLTEHQRTHTGEKPYKCPECG | 1047 |
| | tctgcggctt | | KSFSRNDALTEHQRTHTGEKPYKCPECGKSFSTSGHLVRHQRTHTGEKPYKCPECGKSFSQLAHLRAHQR | |
| | | | THTGEKPYKCPECGKSFSRSDNLVRHQRTHTGEKPYKCPECGKSFSQSSNLVRHQRTHTGKKTS | |
| - | gagagaggtct | 566 | LEPGEKPYKCPECGKSFSTSHSLTEHQRTHTGEKPYKCPECGKSFSTTGALTEHQRTHTGEKPYKCPECGK | 1048 |
| | gcggcttcca | | SFSRSDKLTEHQRTHTGEKPYKCPECGKSFSRNDALTEHQRTHTGEKPYKCPECGKSFSTSGHLVRHQRT | |
| | | | HTGEKPYKCPECGKSFSQLAHLRAHQRTHTGEKPYKCPECGKSFSRSDNLVRHQRTHTGKKTS | |
| - | agaggtctgcg | 567 | LEPGEKPYKCPECGKSFSSKKALTEHQRTHTGEKPYKCPECGKSFSTSHSLTEHQRTHTGEKPYKCPECGK | 1049 |
| | gcttccacac | | SFSTTGALTEHQRTHTGEKPYKCPECGKSFSRSDKLTEHQRTHTGEKPYKCPECGKSFSRNDALTEHQRT | |
| | | | HTGEKPYKCPECGKSFSTSGHLVRHQRTHTGEKPYKCPECGKSFSQLAHLRAHQRTHTGKKTS | |
| - | ggtctgcggctt | 568 | LEPGEKPYKCPECGKSFSSRRTCRAHQRTHTGEKPYKCPECGKSFSSKKALTEHQRTHTGEKPYKCPECG | 1050 |
| | ccacaccgt | | KSFSTSHSLTEHQRTHTGEKPYKCPECGKSFSTTGALTEHQRTHTGEKPYKCPECGKSFSRSDKLTEHQRT | |
| | | | HTGEKPYKCPECGKSFSRNDALTEHQRTHTGEKPYKCPECGKSFSTSGHLVRHQRTHTGKKTS | |
| - | ctgcggcttcca | 569 | LEPGEKPYKCPECGKSFSSPADLTRHQRTHTGEKPYKCPECGKSFSSRRTCRAHQRTHTGEKPYKCPECG | 1051 |
| | caccgtaca | | KSFSSKKALTEHQRTHTGEKPYKCPECGKSFSTSHSLTEHQRTHTGEKPYKCPECGKSFSTTGALTEHQRT | |
| | | | HTGEKPYKCPECGKSFSRSDKLTEHQRTHTGEKPYKCPECGKSFSRNDALTEHQRTHTGKKTS | |
| - | cggcttccacac | 570 | LEPGEKPYKCPECGKSFSRSDDLVRHQRTHTGEKPYKCPECGKSFSSPADLTRHQRTHTGEKPYKCPECG | 1052 |
| | cgtacagcg | | KSFSSRRTCRAHQRTHTGEKPYKCPECGKSFSSKKALTEHQRTHTGEKPYKCPECGKSFSTSHSLTEHQRT | |
| | | | HTGEKPYKCPECGKSFSTTGALTEHQRTHTGEKPYKCPECGKSFSRSDKLTEHQRTHTGKKTS | |
| - | cttccacaccgt | 571 | LEPGEKPYKCPECGKSFSRSDHLTTHQRTHTGEKPYKCPECGKSFSRSDDLVRHQRTHTGEKPYKCPECG | 1053 |
| | acagcgtgg | | KSFSSPADLTRHQRTHTGEKPYKCPECGKSFSSRRTCRAHQRTHTGEKPYKCPECGKSFSSKKALTEHQRT | |
| | | | HTGEKPYKCPECGKSFSTSHSLTEHQRTHTGEKPYKCPECGKSFSTTGALTEHQRTHTGKKTS | |
| - | gcggctTccac | 572 | LEPGEKPYKCPECGKSFSRSDDLVRHQRTHTGEKPYKCPECGKSFSSPADLTRHQRTHTGEKPYKCPECG | 1054 |
| | accgtacagcg | | KSFSSRRTCRAHQRTHTGEKPYKCPECGKSFSSKKALTEHQRTHTGEKPYKCPECGKSFSTSHSLTEHQRT | |
| | | | HTHPRAPIPKPFQYKCPECGKSFSTSGELVRHQRTHTGEKPYKCPECGKSFSRSDDLVRHQRTHTGKKTS | |
| - | gctTccacacc | 573 | LEPGEKPYKCPECGKSFSRSDHLTTHQRTHTGEKPYKCPECGKSFSRSDDLVRHQRTHTGEKPYKCPECG | 1055 |
| | gtacagcgtgg | | KSFSSPADLTRHQRTHTGEKPYKCPECGKSFSSRRTCRAHQRTHTGEKPYKCPECGKSFSSKKALTEHQRT | |
| | | | HTGEKPYKCPECGKSFSTSHSLTEHQRTHTHPRAPIPKPFQYKCPECGKSFSTSGELVRHQRTHTGKKTS | |
| - | ggcTTccaca | 574 | LEPGEKPYKCPECGKSFSRSDHLTTHQRTHTGEKPYKCPECGKSFSRSDDLVRHQRTHTGEKPYKCPECG | 1056 |
| | ccgtacagcgtg | | KSFSSPADLTRHQRTHTGEKPYKCPECGKSFSSRRTCRAHQRTHTGEKPYKCPECGKSFSSKKALTEHQRT | |
| | g | | HTGEKPYKCPECGKSFSTSHSLTEHQRTHTPNPHRRTDPSHKPFQYKCPECGKSFSDPGHLVRHQRTHTG | |
| | | | KKTS | |
| - | cttCTcggTat | 575 | LEPGEKPYKCPECGKSFSTSGSLVRHQRTHTPNPHRRTDPSHKPFQYKCPECGKSFSRKDNLKNHQRTHT | 1057 |
| | aaaagcaaagT | | GEKPYKCPECGKSFSQSGDLRRHQRTHTGEKPYKCPECGKSFSQRANLRAHQRTHTGEKPYKCPECGKSF | |
| | Tgtt | | SQKSSLIAHQRTHTHPRAPIPKPFQYKCPECGKSFSRSDKLTEHQRTHTPNPHRRTDPSHKPFQYKCPECG | |
| | | | KSFSTTGALTEHQRTHTGKKTS | |
| - | cggTataaaag | 576 | LEPGEKPYKCPECGKSFSQAGHLASHQRTHTPNPHRRTDPSHKPFQYKCPECGKSFSTSGSLVRHQRTHTP | 1058 |
| | caaagTTgttT | | NPHRRTDPSHKPFQYKCPECGKSFSRKDNLKNHQRTHTGEKPYKCPECGKSFSQSGDLRRHQRTHTGEKP | |
| | Ttga | | YKCPECGKSFSQRANLRAHQRTHTGEKPYKCPECGKSFSQKSSLIAHQRTHTHPRAPIPKPFQYKCPECGK | |
| | | | SFSRSDKLTEHQRTHTGKKTS | |
| - | ggtataaaagca | 577 | LEPGEKPYKCPECGKSFSQAGHLASHQRTHTPNPHRRTDPSHKPFQYKCPECGKSFSTSGSLVRHQRTHTP | 1059 |
| | aagTTgttTTt | | NPHRRTDPSHKPFQYKCPECGKSFSRKDNLKNHQRTHTGEKPYKCPECGKSFSQSGDLRRHQRTHTGEKP | |
| | ga | | YKCPECGKSFSQRANLRAHQRTHTGEKPYKCPECGKSFSQKSSLIAHQRTHTGEKPYKCPECGKSFSTSG | |
| | | | HLVRHQRTHTGKKTS | |
| - | ataaaagcaaa | 578 | LEPGEKPYKCPECGKSFSRTDTLRDHQRTHTHPRAPIPKPFQYKCPECGKSFSQAGHLASHQRTHTPNPHR | 1060 |
| | gTTgttTTtg | | RTDPSHKPFQYKCPECGKSFSTSGSLVRHQRTHTPNPHRRTDPSHKPFQYKCPECGKSFSRKDNLKNHQR | |
| | aTacg | | THTGEKPYKCPECGKSFSQSGDLRRHQRTHTGEKPYKCPECGKSFSQRANLRAHQRTHTGEKPYKCPEC | |
| | | | GKSFSQKSSLIAHQRTHTGKKTS | |
| - | aaagcaaagT | 579 | LEPGEKPYKCPECGKSFSQAGHLASHQRTHTGEKPYKCPECGKSFSRTDTLRDHQRTHTHPRAPIPKPFQY | 1061 |
| | TgttTTtgaTa | | KCPECGKSFSQAGHLASHQRTHTPNPHRRTDPSHKPFQYKCPECGKSFSTSGSLVRHQRTHTPNPHRRTDP | |
| | cgtga | | SHKPFQYKCPECGKSFSRKDNLKNHQRTHTGEKPYKCPECGKSFSQSGDLRRHQRTHTGEKPYKCPECG | |
| | | | KSFSQRANLRAHQRTHTGKKTS | |
| - | gcaaagTTgtt | 580 | LEPGEKPYKCPECGKSFSRADNLTEHQRTHTGEKPYKCPECGKSFSQAGHLASHQRTHTGEKPYKCPECG | 1062 |
| | TTtgaTacgtg | | KSFSRTDTLRDHQRTHTHPRAPIPKPFQYKCPECGKSFSQAGHLASHQRTHTPNPHRRTDPSHKPFQYKCP | |
| | acag | | ECGKSFSTSGSLVRHQRTHTPNPHRRTDPSHKPFQYKCPECGKSFSRKDNLKNHQRTHTGEKPYKCPECG | |
| | | | KSFSQSGDLRRHQRTHTGKKTS | |
| - | gtaTaaaagca | 581 | LEPGEKPYKCPECGKSFSQAGHLASHQRTHTPNPHRRTDPSHKPFQYKCPECGKSFSTSGSLVRHQRTHT | 1063 |
| | aagttgttTTtg | | GEKPYKCPECGKSFSTSGSLVRHQRTHTGEKPYKCPECGKSFSQRANLRAHQRTHTGEKPYKCPECGKSF | |
| | a | | SERSHLREHQRTHTGEKPYKCPECGKSFSQRANLRAHQRTHTHPRAPIPKPFQYKCPECGKSFSQSSSLVR | |
| | | | HQRTHTGKKTS | |
| - | aaaagcaaagtt | 582 | LEPGEKPYKCPECGKSFSRTDTLRDHQRTHTHPRAPIPKPFQYKCPECGKSFSQAGHLASHQRTHTPNPHR | 1064 |
| | gttTTtgaTac | | RTDPSHKPFQYKCPECGKSFSTSGSLVRHQRTHTGEKPYKCPECGKSFSTSGSLVRHQRTHTGEKPYKCPE | |
| | g | | CGKSFSQRANLRAHQRTHTGEKPYKCPECGKSFSERSHLREHQRTHTGEKPYKCPECGKSFSQRANLRAH | |
| | | | QRTHTGKKTS | |
| - | agcaaagttgtt | 583 | LEPGEKPYKCPECGKSFSQAGHLASHQRTHTGEKPYKCPECGKSFSRTDTLRDHQRTHTHPRAPIPKPFQY | 1065 |
| | TTtgaTacgtg | | KCPECGKSFSQAGHLASHQRTHTPNPHRRTDPSHKPFQYKCPECGKSFSTSGSLVRHQRTHTGEKPYKCP | |
| | a | | ECGKSFSTSGSLVRHQRTHTGEKPYKCPECGKSFSQRANLRAHQRTHTGEKPYKCPECGKSFSERSHLRE | |
| | | | HQRTHTGKKTS | |
| - | aaagttgttTTt | 584 | LEPGEKPYKCPECGKSFSRADNLTEHQRTHTGEKPYKCPECGKSFSQAGHLASHQRTHTGEKPYKCPECG | 1066 |
| | gaTacgtgaca | | KSFSRTDTLRDHQRTHTHPRAPIPKPFQYKCPECGKSFSQAGHLASHQRTHTPNPHRRTDPSHKPFQYKCP | |
| | g | | ECGKSFSTSGSLVRHQRTHTGEKPYKCPECGKSFSTSGSLVRHQRTHTGEKPYKCPECGKSFSQRANLRA | |
| | | | HQRTHTGKKTS | |
| - | aagcaaagtTg | 585 | LEPGEKPYKCPECGKSFSQAGHLASHQRTHTGEKPYKCPECGKSFSRTDTLRDHQRTHTHPRAPIPKPFQY | 1067 |
| | ttTTtgaTacg | | KCPECGKSFSQAGHLASHQRTHTPNPHRRTDPSHKPFQYKCPECGKSFSTSGSLVRHQRTHTHPRAPIPKP | |
| | tga | | FQYKCPECGKSFSHRTTLTNHQRTHTGEKPYKCPECGKSFSQSGNLTEHQRTHTGEKPYKCPECGKSFSR | |
| | | | KDNLKNHQRTHTGKKTS | |
| - | caaagtTgttT | 586 | LEPGEKPYKCPECGKSFSRADNLTEHQRTHTGEKPYKCPECGKSFSQAGHLASHQRTHTGEKPYKCPECG | 1068 |
| | TtgaTacgtga | | KSFSRTDTLRDHQRTHTHPRAPIPKPFQYKCPECGKSFSQAGHLASHQRTHTPNPHRRTDPSHKPFQYKCP | |
| | cag | | ECGKSFSTSGSLVRHQRTHTHPRAPIPKPFQYKCPECGKSFSHRTTLTNHQRTHTGEKPYKCPECGKSFSQ | |
| | | | SGNLTEHQRTHTGKKTS | |
| - | atacgtgacagt | 587 | LEPGEKPYKCPECGKSFSERSHLREHQRTHTGEKPYKCPECGKSFSSPADLTRHQRTHTGEKPYKCPECGK | 1069 |
| | TTcccacaag | | SFSSKKHLAEHQRTHTPNPHRRTDPSHKPFQYKCPECGKSFSHRTTLTNHQRTHTGEKPYKCPECGKSFSD | |
| | c | | PGNLVRHQRTHTGEKPYKCPECGKSFSSRRTCRAHQRTHTGEKPYKCPECGKSFSQKSSLIAHQRTHTGK | |
| | | | KTS | |
| - | cgtgacagtTT | 588 | LEPGEKPYKCPECGKSFSRADNLTEHQRTHTGEKPYKCPECGKSFSERSHLREHQRTHTGEKPYKCPECG | 1070 |
| | cccacaagcca | | KSFSSPADLTRHQRTHTGEKPYKCPECGKSFSSKKHLAEHQRTHTPNPHRRTDPSHKPFQYKCPECGKSFS | |
| | g | | HRTTLTNHQRTHTGEKPYKCPECGKSFSDPGNLVRHQRTHTGEKPYKCPECGKSFSSRRTCRAHQRTHTG | |
| | | | KKTS | |
| - | gacagtTTccc | 589 | LEPGEKPYKCPECGKSFSTSGELVRHQRTHTGEKPYKCPECGKSFSRADNLTEHQRTHTGEKPYKCPECG | 1071 |
| | acaagccaggc | | KSFSERSHLREHQRTHTGEKPYKCPECGKSFSSPADLTRHQRTHTGEKPYKCPECGKSFSSKKHLAEHQRT | |
| | t | | HTPNPHRRTDPSHKPFQYKCPECGKSFSHRTTLTNHQRTHTGEKPYKCPECGKSFSDPGNLVRHQRTHTG | |
| | | | KKTS | |
| - | agtTTcccaca | 590 | LEPGEKPYKCPECGKSFSTSGNLVRHQRTHTGEKPYKCPECGKSFSTSGELVRHQRTHTGEKPYKCPECG | 1072 |
| | agccaggctgat | | KSFSRADNLTEHQRTHTGEKPYKCPECGKSFSERSHLREHQRTHTGEKPYKCPECGKSFSSPADLTRHQRT | |
| | | | HTGEKPYKCPECGKSFSSKKHLAEHQRTHTPNPHRRTDPSHKPFQYKCPECGKSFSHRTTLTNHQRTHTG | |
| | | | KKTS | |
| - | gtgacagttTcc | 591 | LEPGEKPYKCPECGKSFSRADNLTEHQRTHTGEKPYKCPECGKSFSERSHLREHQRTHTGEKPYKCPECG | 1073 |
| | cacaagccag | | KSFSSPADLTRHQRTHTGEKPYKCPECGKSFSSKKHLAEHQRTHTHPRAPIPKPFQYKCPECGKSFSTSGS | |
| | | | LVRHQRTHTGEKPYKCPECGKSFSSPADLTRHQRTHTGEKPYKCPECGKSFSRSDELVRHQRTHTGKKTS | |
| - | acagttTcccac | 592 | LEPGEKPYKCPECGKSFSTSGELVRHQRTHTGEKPYKCPECGKSFSRADNLTEHQRTHTGEKPYKCPECG | 1074 |
| | aagccaggct | | KSFSERSHLREHQRTHTGEKPYKCPECGKSFSSPADLTRHQRTHTGEKPYKCPECGKSFSSKKHLAEHQRT | |
| | | | HTHPRAPIPKPFQYKCPECGKSFSTSGSLVRHQRTHTGEKPYKCPECGKSFSSPADLTRHQRTHTGKKTS | |
| - | gttTcccacaa | 593 | LEPGEKPYKCPECGKSFSTSGNLVRHQRTHTGEKPYKCPECGKSFSTSGELVRHQRTHTGEKPYKCPECG | 1075 |
| | gccaggctgat | | KSFSRADNLTEHQRTHTGEKPYKCPECGKSFSERSHLREHQRTHTGEKPYKCPECGKSFSSPADLTRHQRT | |
| | | | HTGEKPYKCPECGKSFSSKKHLAEHQRTHTHPRAPIPKPFQYKCPECGKSFSTSGSLVRHQRTHTGKKTS | |
| - | cccacaagcca | 594 | LEPGEKPYKCPECGKSFSTKNSLTEHQRTHTGEKPYKCPECGKSFSTSGNLVRHQRTHTGEKPYKCPECG | 1076 |
| | ggctgatcct | | KSFSTSGELVRHQRTHTGEKPYKCPECGKSFSRADNLTEHQRTHTGEKPYKCPECGKSFSERSHLREHQR | |
| | | | THTGEKPYKCPECGKSFSSPADLTRHQRTHTGEKPYKCPECGKSFSSKKHLAEHQRTHTGKKTS | |
| - | cttTTctgTca | 595 | LEPGEKPYKCPECGKSFSQSGNLTEHQRTHTGEKPYKCPECGKSFSSKKALTEHQRTHTGEKPYKCPECG | 1077 |
| | gTccacttcacc | | KSFSTTGALTEHQRTHTGEKPYKCPECGKSFSTSHSLTEHQRTHTHPRAPIPKPFQYKCPECGKSFSRADN | |
| | aa | | LTEHQRTHTHPRAPIPKPFQYKCPECGKSFSRNDALTEHQRTHTPNPHRRTDPSHKPFQYKCPECGKSFST | |
| | | | TGALTEHQRTHTGKKTS | |
| - | ctgTcagTcca | 596 | LEPGEKPYKCPECGKSFSTSGHLVRHQRTHTGEKPYKCPECGKSFSQSGNLTEHQRTHTGEKPYKCPECG | 1078 |
| | cttcaccaaggt | | KSFSSKKALTEHQRTHTGEKPYKCPECGKSFSTTGALTEHQRTHTGEKPYKCPECGKSFSTSHSLTEHQRT | |
| | | | HTHPRAPIPKPFQYKCPECGKSFSRADNLTEHQRTHTHPRAPIPKPFQYKCPECGKSFSRNDALTEHQRTH | |
| | | | TGKKTS | |
| - | gtcagtccacttc | 597 | LEPGEKPYKCPECGKSFSTSGHLVRHQRTHTGEKPYKCPECGKSFSQSGNLTEHQRTHTGEKPYKCPECG | 1079 |
| | accaaggt | | KSFSSKKALTEHQRTHTGEKPYKCPECGKSFSTTGALTEHQRTHTGEKPYKCPECGKSFSTSHSLTEHQRT | |
| | | | HTGEKPYKCPECGKSFSHRTTLTNHQRTHTGEKPYKCPECGKSFSDPGALVRHQRTHTGKKTS | |
| - | agtccacttcac | 598 | LEPGEKPYKCPECGKSFSRSDNLVRHQRTHTGEKPYKCPECGKSFSTSGHLVRHQRTHTGEKPYKCPECG | 1080 |
| | caaggtgag | | KSFSQSGNLTEHQRTHTGEKPYKCPECGKSFSSKKALTEHQRTHTGEKPYKCPECGKSFSTTGALTEHQR | |
| | | | THTGEKPYKCPECGKSFSTSHSLTEHQRTHTGEKPYKCPECGKSFSHRTTLTNHQRTHTGKKTS | |
| - | ccacttcaccaa | 599 | LEPGEKPYKCPECGKSFSDPGALVRHQRTHTHPRAPIPKPFQYKCPECGKSFSRSDNLVRHQRTHTGEKPY | 1081 |
| | ggtgagTgtc | | KCPECGKSFSTSGHLVRHQRTHTGEKPYKCPECGKSFSQSGNLTEHQRTHTGEKPYKCPECGKSFSSKKA | |
| | | | LTEHQRTHTGEKPYKCPECGKSFSTTGALTEHQRTHTGEKPYKCPECGKSFSTSHSLTEHQRTHTGKKTS | |
| - | cttcaccaaggt | 600 | LEPGEKPYKCPECGKSFSTKNSLTEHQRTHTGEKPYKCPECGKSFSDPGALVRHQRTHTHPRAPIPKPFQY | 1082 |
| | gagTgtccct | | KCPECGKSFSRSDNLVRHQRTHTGEKPYKCPECGKSFSTSGHLVRHQRTHTGEKPYKCPECGKSFSQSGN | |
| | | | LTEHQRTHTGEKPYKCPECGKSFSSKKALTEHQRTHTGEKPYKCPECGKSFSTTGALTEHQRTHTGKKTS | |
| - | caccaaggtga | 601 | LEPGEKPYKCPECGKSFSTSGELVRHQRTHTGEKPYKCPECGKSFSTKNSLTEHQRTHTGEKPYKCPECG | 1083 |
| | gTgtccctgct | | KSFSDPGALVRHQRTHTHPRAPIPKPFQYKCPECGKSFSRSDNLVRHQRTHTGEKPYKCPECGKSFSTSGH | |
| | | | LVRHQRTHTGEKPYKCPECGKSFSQSGNLTEHQRTHTGEKPYKCPECGKSFSSKKALTEHQRTHTGKKTS | |
| - | caaggtgagTg | 602 | LEPGEKPYKCPECGKSFSSKKHLAEHQRTHTPNPHRRTDPSHKPFQYKCPECGKSFSTSGELVRHQRTHTG | 1084 |
| | tccctgctCTc | | EKPYKCPECGKSFSTKNSLTEHQRTHTGEKPYKCPECGKSFSDPGALVRHQRTHTHPRAPIPKPFQYKCPE | |
| | cc | | CGKSFSRSDNLVRHQRTHTGEKPYKCPECGKSFSTSGHLVRHQRTHTGEKPYKCPECGKSFSQSGNLTEH | |
| | | | QRTHTGKKTS | |
| - | ggtgagTgtcc | 603 | LEPGEKPYKCPECGKSFSQNSTLTEHQRTHTGEKPYKCPECGKSFSSKKHLAEHQRTHTPNPHRRTDPSHK | 1085 |
| | ctgctCTcccc | | PFQYKCPECGKSFSTSGELVRHQRTHTGEKPYKCPECGKSFSTKNSLTEHQRTHTGEKPYKCPECGKSFSD | |
| | ta | | PGALVRHQRTHTHPRAPIPKPFQYKCPECGKSFSRSDNLVRHQRTHTGEKPYKCPECGKSFSTSGHLVRH | |
| | | | QRTHTGKKTS | |
| - | gagTgtccctg | 604 | LEPGEKPYKCPECGKSFSTSHSLTEHQRTHTGEKPYKCPECGKSFSQNSTLTEHQRTHTGEKPYKCPECGK | 1086 |
| | ctCTcccctac | | SFSSKKHLAEHQRTHTPNPHRRTDPSHKPFQYKCPECGKSFSTSGELVRHQRTHTGEKPYKCPECGKSFST | |
| | ca | | KNSLTEHQRTHTGEKPYKCPECGKSFSDPGALVRHQRTHTHPRAPIPKPFQYKCPECGKSFSRSDNLVRH | |
| | | | QRTHTGKKTS | |
| - | cagTccacttca | 605 | LEPGEKPYKCPECGKSFSRSDNLVRHQRTHTGEKPYKCPECGKSFSTSGHLVRHQRTHTGEKPYKCPECG | 1087 |
| | ccaaggtgag | | KSFSQSGNLTEHQRTHTGEKPYKCPECGKSFSSKKALTEHQRTHTGEKPYKCPECGKSFSTTGALTEHQR | |
| | | | THTGEKPYKCPECGKSFSTSHSLTEHQRTHTHPRAPIPKPFQYKCPECGKSFSRADNLTEHQRTHTGKKTS | |
| - | gtccacTTcac | 606 | LEPGEKPYKCPECGKSFSDPGALVRHQRTHTHPRAPIPKPFQYKCPECGKSFSRSDNLVRHQRTHTGEKPY | 1088 |
| | caaggtgagTg | | KCPECGKSFSTSGHLVRHQRTHTGEKPYKCPECGKSFSQSGNLTEHQRTHTGEKPYKCPECGKSFSSKKA | |
| | tc | | LTEHQRTHTPNPHRRTDPSHKPFQYKCPECGKSFSSKKALTEHQRTHTGEKPYKCPECGKSFSDPGALVR | |
| | | | HQRTHTGKKTS | |
| - | cacTTcaccaa | 607 | LEPGEKPYKCPECGKSFSTKNSLTEHQRTHTGEKPYKCPECGKSFSDPGALVRHQRTHTHPRAPIPKPFQY | 1089 |
| | ggtgagTgtcc | | KCPECGKSFSRSDNLVRHQRTHTGEKPYKCPECGKSFSTSGHLVRHQRTHTGEKPYKCPECGKSFSQSGN | |
| | ct | | LTEHQRTHTGEKPYKCPECGKSFSSKKALTEHQRTHTPNPHRRTDPSHKPFQYKCPECGKSFSSKKALTEH | |
| | | | QRTHTGKKTS | |
| - | actTcaccaag | 608 | LEPGEKPYKCPECGKSFSTKNSLTEHQRTHTGEKPYKCPECGKSFSDPGALVRHQRTHTHPRAPIPKPFQY | 1090 |
| | gtgagTgtccct | | KCPECGKSFSRSDNLVRHQRTHTGEKPYKCPECGKSFSTSGHLVRHQRTHTGEKPYKCPECGKSFSQSGN | |
| | | | LTEHQRTHTGEKPYKCPECGKSFSSKKALTEHQRTHTHPRAPIPKPFQYKCPECGKSFSTHLDLIRHQRTH | |
| | | | TGKKTS | |

Similarly, the design and preparation of such TALE polypeptides which specifically bind to a DNA target region of interest, such as a FOXP3 expression control region, is well known in the art. For example, the TALE DNA binding domain contains a repeated highly conserved 33-34 amino acid sequence with divergent 12th and 13th amino acids. These two positions, referred to as the Repeat Variable Diresidue (RVD), are highly variable and show a strong correlation with specific nucleotide recognition. This straightforward relationship between amino acid sequence and DNA recognition has allowed for the engineering of specific DNA-binding domains by selecting a combination of repeat segments containing the appropriate RVDs. See, e.g., Boch J Nature Biotechnology. 29 (2) 2011: 135-6; Boch J, et al., Science. 326 (5959) 2009: 1509-12; Moscou M J & Bogdanove A J Science. 326 (5959) 2009: 1501.

In some embodiments, the site-specific FOXP3 targeting moieties of the invention comprising a polynucleotide comprise a guide RNA (or gRNA) or nucleic acid encoding a guide RNA. A gRNA is a short synthetic RNA molecule comprising a “scaffold” sequence necessary for, e.g., directing an effector to a FOXP3 expression control element which may, e.g., include an about 20 nucleotide site-specific sequence targeting a genomic target sequence comprising the FOXP3 expression control element.

Generally, guide RNA sequences are designed to have a length of between about 17 to about 24 nucleotides (e.g., 19,20, or 21 nucleotides) and are complementary to the target sequence. Custom gRNA generators and algorithms are available commercially for use in the design of effective guide RNAs. Gene editing has also been achieved using a chimeric “single guide RNA” (“sgRNA”), an engineered (synthetic) single RNA molecule that mimics a naturally occurring crRNA-tracrRNA complex and contains both a tracrRNA (for binding the nuclease) and at least one crRNA (to guide the nuclease to the sequence targeted for editing). Chemically modified sgNAs have also been demonstrated to be effective in genome editing; see, for example, Hendel et al. (2015) Nature Biotechnol., 985-991.

In certain embodiments, the site-specific FOXP3 targeting moieties of the invention comprise a guide RNA (or gRNA) or nucleic acid encoding a guide RNA and a protein or a peptide. In some embodiment, the protein or the peptide comprises a CRISPR associated protein (Cas) polypeptide, or fragment thereof (e.g., a Cas9 polypeptide, or fragment thereof). In one embodiment, a suitable Cas polypeptide is an enzymatically inactive Cas polypeptide, e.g., a “dead Cas polypeptide” or “dCas” polypeptide.

Exemplary site-specific FOXP3 targeting moieties comprising a polynucleotide, e.g., gRNA, are provided in Table 2, below. In some embodiments, the polynucleotide comprises a nucleotide sequence at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical to the entire nucleotide sequence of any one of the nucleotide sequences in Table 2.

It will be understood that, although the sequences in Table 2 are described as modified (or unmodified), the nucleic acid molecules encompassed by the invention, e.g., a site-specific disrupting agent, may comprise any one of the sequences set forth in Table 2 that is unmodified or modified differently than described therein. It will also be understood that although some of the sequences in Table 2 have “Ts”, when used as an RNA molecule, such as a guide RNA, in the site-specific targeting moieties of the invention, the “Ts” may be replaced with “Us.”

In some embodiments, a site-specific FOXP3 targeting moiety comprising a polynucleotide, e.g., gRNA, comprises a nucleotide sequence complementary to an anchor sequence. In one embodiment, the anchor sequence comprises a CTCF-binding motif or consensus sequence: N(T/C/G)N(G/A/T)CC(A/T/G)(C/G)(C/T/A)AG(G/A)(G/T)GG(C/A/T)(G/A)(C/G)(C/T/A)(G/A/C) (SEQ ID NO: 1), where N is any nucleotide. A CTCF-binding motif or consensus sequence may also be in the opposite orientation, e.g., (G/A/C)(C/T/A)(C/G)(G/A)(C/A/T)GG(G/T)(G/A)GA(C/T/A)(C/G)(A/T/G)CC(G/A/T)N(T/C/G)N (SEQ ID NO: 2). In some embodiments, the nucleic acid sequence comprises a sequence complementary to a CTCF-binding motif or consensus sequence.

In some embodiments, the polynucleotide comprises a nucleotide sequence at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% complementary to an anchor sequence.

In some embodiments, the polynucleotide comprises a nucleotide sequence at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% complementary to a CTCF-binding motif or consensus sequence. In some embodiments, the polynucleotide is selected from the group consisting of a gRNA, and a sequence complementary or a sequence comprising at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% complementary sequence to an anchor sequence.

In some embodiments, a site-specific FOXP3 targeting moiety comprising a polynucleotide of the invention is an RNAi molecule. RNAi molecules comprise RNA or RNA-like structures typically containing 15-50 base pairs (such as about 18-25 base pairs) and having a nucleobase sequence identical (complementary) or nearly identical (substantially complementary) to a coding sequence in an expressed target gene within the cell. RNAi molecules include, but are not limited to: short interfering RNAs (siRNAs), double-strand RNAs (dsRNA), micro RNAs (miRNAs), short hairpin RNAs (shRNA), meroduplexes, and dicer substrates (U.S. Pat. Nos. 8,084,599, 8,349,809, and 8,513,207). In one embodiment, the invention includes a composition to inhibit expression of a gene encoding a polypeptide described herein, e.g., a conjunction nucleating molecule.

RNAi molecules comprise a sequence substantially complementary, or fully complementary, to all or a fragment of a target gene. RNAi molecules may complement sequences at the boundary between introns and exons to prevent the maturation of newly-generated nuclear RNA transcripts of specific genes into mRNA for transcription. RNAi molecules complementary to specific genes can hybridize with the mRNA for that gene and prevent its translation. The antisense molecule can be DNA, RNA, or a derivative or hybrid thereof. Examples of such derivative molecules include, but are not limited to, peptide nucleic acid (PNA) and phosphorothioate-based molecules such as deoxyribonucleic guanidine (DNG) or ribonucleic guanidine (R G).

RNAi molecules can be provided to the cell as “ready-to-use” RNA synthesized in vitro or as an antisense gene transfected into cells which will yield RNAi molecules upon transcription. Hybridization with mRNA results in degradation of the hybridized molecule by RNAse H and/or inhibition of the formation of translation complexes. Both result in a failure to produce the product of the original gene.

The length of the RNAi molecule that hybridizes to the transcript of interest should be around 10 nucleotides, between about 15 or 30 nucleotides, or about 15, 16, 17, 18, 19, 20, 21, 22, 23, 24, 25, 26, 27, 28, 29, 30 or more nucleotides. The degree of identity of the antisense sequence to the targeted transcript should be at least 75%, at least 80%, at least 85%, at least 90%, or at least 95.

RNAi molecules may also comprise overhangs, i.e. typically unpaired, overhanging nucleotides which are not directly involved in the double helical structure normally formed by the core sequences of the herein defined pair of sense strand and antisense strand. RNAi molecules may contain 3′ and/or 5′ overhangs of about 1-5 bases independently on each of the sense strands and antisense strands. In one embodiment, both the sense strand and the antisense strand contain 3′ and 5′ overhangs. In one embodiment, one or more of the 3′ overhang nucleotides of one strand base pairs with one or more 5′ overhang nucleotides of the other strand. In another embodiment, the one or more of the 3′ overhang nucleotides of one strand base do not pair with the one or more 5′ overhang nucleotides of the other strand. The sense and antisense strands of an RNAi molecule may or may not contain the same number of nucleotide bases. The antisense and sense strands may form a duplex wherein the 5′ end only has a blunt end, the 3′ end only has a blunt end, both the 5′ and 3′ ends are blunt ended, or neither the 5′ end nor the 3′ end are blunt ended. In another embodiment, one or more of the nucleotides in the overhang contains a thiophosphate, phosphorothioate, deoxynucleotide inverted (3′ to 3′ linked) nucleotide or is a modified ribonucleotide or deoxynucleotide.

Small interfering RNA (siRNA) molecules comprise a nucleotide sequence that is identical to about 15 to about 25 contiguous nucleotides of the target mRNA. In some embodiments, the siRNA sequence commences with the dinucleotide AA, comprises a GC-content of about 30-70% (about 50-60%, about 40-60%, or about 45%-55%), and does not have a high percentage identity to any nucleotide sequence other than the target in the genome of the mammal in which it is to be introduced, for example as determined by standard BLAST search.

siRNAs and shRNAs resemble intermediates in the processing pathway of the endogenous microRNA (miRNA) genes (Bartel, Cell 116:281-297, 2004). In some embodiments, siRNAs can function as miRNAs and vice versa (Zeng et al., Mol Cell 9: 1327-1333, 2002; Doench et al., Genes Dev 17:438-442, 2003). MicroRNAs, like siRNAs, use RISC to downregulate target genes, but unlike siRNAs, most animal miRNAs do not cleave the mRNA. Instead, miRNAs reduce protein output through translational suppression or polyA removal and mRNA degradation (Wu et al., Proc Natl Acad Sci USA 103:4034-4039, 2006). Known miRNA binding sites are within mRNA 3′ UTRs; miRNAs seem to target sites with near-perfect complementarity to nucleotides 2-8 from the miRNA's 5′ end (Rajewsky, Nat Genet 38 Suppl: S8-13, 2006; Lim et al, Nature 433:769-773, 2005). This region is known as the seed region. Because siRNAs and miRNAs are interchangeable, exogenous siRNAs downregulate mRNAs with seed complementarity to the siRNA (Birmingham et al., Nat Methods 3: 199-204, 2006. Multiple target sites within a 3′ UTR give stronger downregulation (Doench et al., Genes Dev 17:438-442, 2003).

Lists of known miRNA sequences can be found in databases maintained by research organizations, such as Wellcome Trust Sanger Institute, Perm Center for Bioinformatics, Memorial Sloan Kettering Cancer Center, and European Molecule Biology Laboratory, among others. Known effective siRNA sequences and cognate binding sites are also well represented in the relevant literature. RNAi molecules are readily designed and produced by technologies known in the art. In addition, there are computational tools that increase the chance of finding effective and specific sequence motifs (Pei et al. 2006, Reynolds et al. 2004, Khvorova et al. 2003, Schwarz et al. 2003, Ui-Tei et al. 2004, Heale et al. 2005, Chalk et al. 2004, Amarzguioui et al. 2004).

An RNAi molecule modulates expression of RNA encoded by a gene. Because multiple genes can share some degree of sequence homology with each other, in some embodiments, the RNAi molecule can be designed to target a class of genes with sufficient sequence homology. In some embodiments, the RNAi molecule can contain a sequence that has complementarity to sequences that are shared amongst different gene targets or are unique for a specific gene target. In some embodiments, the RNAi molecule can be designed to target conserved regions of an RNA sequence having homology between several genes thereby targeting several genes in a gene family (e.g., different gene isoforms, splice variants, mutant genes, etc.). In some embodiments, the RNAi molecule can be designed to target a sequence that is unique to a specific RNA sequence of a single gene.

In some embodiments, the RNAi molecule targets a sequence in a conjunction nucleating molecule, e.g., CTCF, cohesin, USF 1, YY1, TATA-box binding protein associated factor 3 (TAF3), ZNF 143, or another polypeptide that promotes the formation of an anchor sequence-mediated conjunction, or an epigenetic modifying agent, e.g., an enzyme involved in post-translational modifications including, but are not limited to, DNA methylases (e.g., DNMT3a, DNMT3b, DNMTL), DNA demethylation (e.g., the TET family enzymes catalyze oxidation of 5-methylcytosine to 5-hydroxymethylcytosine and higher oxidative derivatives), histone methyltransferases, histone deacetylase (e.g., HDAC1, HDAC2, HDAC3), sirtuin 1, 2, 3, 4, 5, 6, or 7, lysine-specific histone demethylase 1 (LSD1), histone-lysine-N-methyltransferase (Setdbl), euchromatic histone-lysine N-methyltransferase 2 (G9a), histone-lysine N-methyltransferase (SUV39H1), enhancer of zeste homolog 2 (EZH2), viral lysine methyltransferase (vSET), histone methyltransferase (SET2), protein-lysine N-methyltransferase (SMYD2), and others. In one embodiment, the RNAi molecule targets a protein deacetylase, e.g., sirtuin 1, 2, 3, 4, 5, 6, or 7. In one embodiment, the invention includes a composition comprising an RNAi that targets a conjunction nucleating molecule, e.g., CTCF.

In some embodiments, the site-specific FOXP3 targeting moiety comprises a peptide or protein moiety. In some embodiments, a site-specific disrupting agent comprises a fusion protein. In some embodiments, an effector is a peptide or protein moiety. The peptide or protein moieties may include, but is not limited to, a peptide ligand, antibody fragment, or targeting aptamer that binds a receptor such as an extracellular receptor, neuropeptide, hormone peptide, peptide drug, toxic peptide, viral or microbial peptide, synthetic peptide, and agonist or antagonist peptide.

Exemplary peptides or protein include a DNA-binding protein, a CRISPR component protein, a conjunction nucleating molecule, a dominant negative conjunction nucleating molecule, an epigenetic modifying agent, or any combination thereof. In some embodiments, the peptide comprises a nuclease, a physical blocker, an epigenetic recruiter, and an epigenetic CpG modifier, and fragments and combinations of any of the foregoing. In some embodiments, the peptide comprises a DNA-binding domain of a protein, such as a helix-turn-helix motif, a leucine zipper, a Zn-finger, a TATA box binding proteins, a transcription factor.

Peptides or proteins may be linear or branched. The peptide or protein moiety may have a length from about 5 to about 200 amino acids, about 15 to about 150 amino acids, about 20 to about 125 amino acids, about 25 to about 100 amino acids, about 20-70 amino acids, about 20-80 amino acids, about 20-90 amino acids, about 30-100 amino acids, about 30-60 amino acids, about 30-80 amino acids, about 35-85 amino acids, about 40-100 amino acids, or about 50-125 amino acids or any range therebetween.

As indicated above, in some embodiments, the site-specific FOXP3 targeting moieties of the invention comprise a fusion protein.

In some embodiments, the fusion proteins of the invention include a site-specific FOXP3 targeting moiety which targets a FOXP3 expression control region and an effector molecule. In other embodiments, a fusion protein of the invention comprises an effector molecule. Exemplary effector molecules are described below and in some embodiments include, for example, nucleases, physical blockers, epigenetic recruiters, e.g., a transcriptional enhancer or a transcriptional repressor, and epigenetic CpG modifiers, e.g., a DNA methylase, a DNA demethylase, a histone modifying agent, a histone transacetylase, or a histone deacetylase, and combinations of any of the foregoing.

For example, a site-specific targeting moiety may comprise a gRNA and an effector, such as a nuclease, e.g., a Cas9, e.g., a wild type Cas9, a nickase Cas9 (e.g., Cas9 D10A), a dead Cas9 (dCas9), eSpCas9, Cpf1, C2C1, or C2C3, or a nucleic acid encoding such a nuclease. The choice of nuclease and gRNA(s) is determined by whether the targeted mutation is a deletion, substitution, or addition of nucleotides, e.g., a deletion, substitution, or addition of nucleotides to a target sequence. Fusions of a catalytically inactive endonuclease e.g., a dead Cas9 (dCas9, e.g., D10A; H840A) tethered with all or a portion of (e.g., biologically active portion of) an (one or more) effector domain create chimeric proteins that can be linked to the polypeptide to guide the composition to specific DNA sites by one or more RNA sequences (e.g., DNA recognition elements including, but not restricted to zinc finger arrays, sgRNA, TAL arrays, peptide nucleic acids described herein) to modulate activity and/or expression of one or more target nucleic acids sequences (e.g., to methylate or demethylate a DNA sequence).

In one embodiment, a fusion protein of the invention may comprise an effector molecule comprising, for example, a CRISPR associated protein (Cas) polypeptide, or fragment thereof, (e.g., a Cas9 polypeptide, or fragment thereof) and an epigenetic recruiter or an epigenetic CpG modifier.

In one embodiment, a suitable Cas polypeptide is an enzymatically inactive Cas polypeptide, e.g., a “dead Cas polypeptide” or “dCas” polypeptide

Exemplary Cas polypeptides that are adaptable to the methods and compositions described herein are described below. Using methods known in the art, a Cas polypeptide can be fused to any of a variety of agents and/or molecules as described herein; such resulting fusion molecules can be useful in various disclosed methods.

In one aspect, the invention includes a composition comprising a protein comprising a domain, e.g., an effector, that acts on DNA (e.g., a nuclease domain, e.g., a Cas9 domain, e.g., a dCas9 domain; a DNA methyltransferase, a demethylase, a deaminase), in combination with at least one guide RNA (gRNA) or antisense DNA oligonucleotide that targets the protein to site-specific target sequence, wherein the composition is effective to alter, in a human cell, the expression of a target gene. In some embodiments, the enzyme domain is a Cas9 or a dCas9. In some embodiments, the protein comprises two enzyme domains, e.g., a dCas9 and a methylase or demethylase domain.

In one aspect, the invention includes a composition comprising a protein comprising a domain, e.g., an effector, that comprises a transcriptional control element (e.g., a nuclease domain, e.g., a Cas9 domain, e.g., a dCas9 domain; a transcriptional enhancer; a transcriptional repressor), in combination with at least one guide RNA (gRNA) or antisense DNA oligonucleotide that targets the protein to a site-specific target sequence, wherein the composition is effective to alter, in a human cell, the expression of a target gene. In some embodiments, the enzyme domain is a Cas9 or a dCas9. In some embodiments, the protein comprises two enzyme domains, e.g., a dCas9 and a transcriptional enhancer or transcriptional repressor domain.

As used herein, a “biologically active portion of an effector domain” is a portion that maintains the function (e.g. completely, partially, minimally) of an effector domain (e.g., a “minimal” or “core” domain).

The chimeric proteins described herein may also comprise a linker, e.g., an amino acid linker. In some aspects, a linker comprises 2 or more amino acids, e.g., one or more GS sequences. In some aspects, fusion of Cas9 (e.g., dCas9) with two or more effector domains (e.g., of a DNA methylase or enzyme with a role in DNA demethylation or protein acetyl transferase or deacetylase) comprises one or more interspersed linkers (e.g., GS linkers) between the domains. In some aspects, dCas9 is fused with 2-5 effector domains with interspersed linkers.

In some embodiments, a site-specific FOXP3 targeting moiety comprises a conjunction nucleating molecule, a nucleic acid encoding a conjunction nucleating molecule, or a combination thereof. In some embodiments, an anchor sequence-mediated conjunction is mediated by a first conjunction nucleating molecule bound to the first anchor sequence, a second conjunction nucleating molecule bound to the noncontiguous second anchor sequence, and an association between the first and second conjunction nucleating molecules. In some embodiments, a conjunction nucleating molecule may disrupt, e.g., by competitive binding, the binding of an endogenous conjunction nucleating molecule to its binding site.

The conjunction nucleating molecule may be, e.g., CTCF, cohesin, USF1, YY1, TATA-box binding protein associated factor 3 (TAF3), ZNF143 binding motif, or another polypeptide that promotes the formation of an anchor sequence-mediated conjunction. The conjunction nucleating molecule may be an endogenous polypeptide or other protein, such as a transcription factor, e.g., autoimmune regulator (AIRE), another factor, e.g., X-inactivation specific transcript (XIST), or an engineered polypeptide that is engineered to recognize a specific DNA sequence of interest, e.g., having a zinc finger, leucine zipper or bHLH domain for sequence recognition. The conjunction nucleating molecule may modulate DNA interactions within or around the anchor sequence-mediated conjunction. For example, the conjunction nucleating molecule can recruit other factors to the anchor sequence that alters an anchor sequence-mediated conjunction formation or disruption.

The conjunction nucleating molecule may also have a dimerization domain for homo- or heterodimerization. One or more conjunction nucleating molecules, e.g., endogenous and engineered, may interact to form the anchor sequence-mediated conjunction. In some embodiments, the conjunction nucleating molecule is engineered to further include a stabilization domain, e.g., cohesion interaction domain, to stabilize the anchor sequence-mediated conjunction. In some embodiments, the conjunction nucleating molecule is engineered to bind a target sequence, e.g., target sequence binding affinity is modulated. In some embodiments, the conjunction nucleating molecule is selected or engineered with a selected binding affinity for an anchor sequence within the anchor sequence-mediated conjunction. Conjunction nucleating molecules and their corresponding anchor sequences may be identified through the use of cells that harbor inactivating mutations in CTCF and Chromosome Conformation Capture or 3C-based methods, e.g., Hi-C or high-throughput sequencing, to examine topologically associated domains, e.g., topological interactions between distal DNA regions or loci, in the absence of CTCF. Long-range DNA interactions may also be identified. Additional analyses may include Ch1A-PET analysis using a bait, such as Cohesin, YY1 or USF1, ZNF143 binding motif, and MS to identify complexes that are associated with the bait.

B. Effector Molecules

Effector molecules for use in the compositions and methods of the invention include those that modulate a biological activity, for example increasing or decreasing enzymatic activity, gene expression, cell signaling, and cellular or organ function. Preferred effector molecules of the invention are nucleases, physical blockers, epigenetic recruiters, e.g., a transcriptional enhancer or a transcriptional repressor, and epigenetic CpG modifiers, e.g., a DNA methylase, a DNA demethylase, a histone modifying agent, a histone transacetylase, or a histone deacetylase, and combinations of any of the foregoing.

Additional effector activities may also include binding regulatory proteins to modulate activity of the regulator, such as transcription or translation. Effector molecules also may include activator or inhibitor (or “negative effector”) functions as described herein. In another example, the effector molecule may inhibit substrate binding to a receptor and inhibit its activation, e.g., naltrexone and naloxone bind opioid receptors without activating them and block the receptors' ability to bind opioids. Effector molecules may also modulate protein stability/degradation and/or transcript stability/degradation. For example, proteins may be targeted for degradation by the polypeptide co-factor, ubiquitin, onto proteins to mark them for degradation. In another example, an effector molecule inhibits enzymatic activity by blocking the enzyme's active site, e.g., methotrexate is a structural analog of tetrahydrofolate, a coenzyme for the enzyme dihydrofolate reductase that binds to dihydrofolate reductase 1000-fold more tightly than the natural substrate and inhibits nucleotide base synthesis.

In some embodiments, the effector molecule is a chemical, e.g., a chemical that modulates a cytosine (C) or an adenine(A) (e.g., Na bisulfite, ammonium bisulfite). In some embodiments, the effector molecule has enzymatic activity (methyl transferase, demethylase, nuclease (e.g., Cas9), a deaminase). In some embodiments, the effector molecule sterically hinders formation of an anchor sequence-mediated conjunction or binding of an RNA polymerase to a promoter.

The effector molecule with effector activity may be any one of the small molecules, peptides, fusion proteins, nucleic acids, nanoparticle, aptamers, or pharmacoagents with poor PK/PD described herein.

In some embodiments, the effector molecule is an inhibitor or “negative effector molecule”. In the context of a negative effector molecule that modulates formation of an anchor sequence-mediated conjunction, in some embodiments, the negative effector molecule is characterized in that dimerization of an endogenous nucleating polypeptide is reduced when the negative effector molecule is present as compared with when it is absent. For example, in some embodiments, the negative effector molecule is or comprises a variant of the endogenous nucleating polypeptide's dimerization domain, or a dimerizing portion thereof.

For example, in certain embodiments, an anchor sequence-mediated conjunction is altered (e.g., disrupted) by use of a dominant negative effector, e.g., a protein that recognizes and binds an anchor sequence, (e.g., a CTCF binding motif), but with an inactive (e.g., mutated) dimerization domain, e.g., a dimerization domain that is unable to form a functional anchor sequence-mediated conjunction. For example, the Zinc Finger domain of CTCF can be altered so that it binds a specific anchor sequence (by adding zinc fingers that recognize flanking nucleic acids), while the homo-dimerization domain is altered to prevent the interaction between the engineered CTCF and endogenous forms of CTCF.

In some embodiments, the effector molecule comprises a synthetic conjunction nucleating molecule with a selected binding affinity for an anchor sequence within a target anchor sequence-mediated conjunction, (the binding affinity may be at least 10%, 15%, 20%, 25%, 30%, 35%, 40%, 45%, 50%, 55%, 60%, 65%, 70%, 75%, 80%, 85%, 90%, 95%, or higher or lower than the affinity of an endogenous conjunction nucleating molecule that associates with the target anchor sequence. The synthetic conjunction nucleating molecule may have between 30-90%, 30-85%, 30-80%, 30-70%, 50-80%, 50-90% amino acid sequence identity to the endogenous conjunction nucleating molecule). The conjunction nucleating molecule may disrupt, such as through competitive binding, the binding of an endogenous conjunction nucleating molecule to its anchor sequence. In some more embodiments, the conjunction nucleating molecule is engineered to bind a novel anchor sequence within the anchor sequence-mediated conjunction.

In some embodiments, a dominant negative effector molecule has a domain that recognizes specific DNA sequences (e.g., an anchor sequence, a CTCF anchor sequence, flanked by sequences that confer sequence specificity), and a second domain that provides a steric presence in the vicinity of the anchoring sequence. The second domain may include a dominant negative conjunction nucleating molecule or fragment thereof, a polypeptide that interferes with conjunction nucleating molecule sequence recognition (e.g., the amino acid backbone of a peptide/nucleic acid or PNA), a nucleic acid sequence ligated to a small molecule that imparts steric interference, or any other combination of DNA recognition elements and a steric blocker.

In some embodiments, the effector molecule is an epigenetic modifying agent. Epigenetic modifying agents useful in the methods and compositions described herein include agents that affect, e.g., DNA methylation/demethylation, histone acetylation/deacetylation, and RNA-associated silencing. In some embodiments, the effectors sequence-specifically target an epigenetic enzyme (e.g., an enzyme that generates or removes epigenetic marks, e.g., acetylation and/or methylation). Exemplary epigenetic effectors may target an expression control region comprising, e.g., a transcriptional control element or an anchor sequence, by a site-specific disrupting agent comprising a site-specific targeting moiety.

In some embodiments, an effector molecule comprises one or more components of a gene editing system. Components of gene editing systems may be used in a variety of contexts including but not limited to gene editing. For example, such components may be used to target agents that physically modify, genetically modify, and/or epigenetically modify FOXP3 sequences.

Exemplary gene editing systems include the clustered regulatory interspaced short palindromic repeat (CRISPR) system, zinc finger nucleases (ZFNs), and Transcription Activator-Like Effector-based Nucleases (TALEN). ZFNs, TALENs, and CRISPR-based methods are described, e.g., in Gaj et al. Trends Biotechnol. 31.7(2013):397-405; CRISPR methods of gene editing are described, e.g., in Guan et al, Application of CRISPR-Cas system in gene therapy: Pre-clinical progress in animal model. DNA Repair 2016 Jul. 30 [Epub ahead of print]; Zheng et al, Precise gene deletion and replacement using the CRISPR/Cas9 system in human cells. BioTechniques, Vol. 57, No. 3, September 2014, pp. 115-124.

CRISPR systems are adaptive defense systems originally discovered in bacteria and archaea. CRISPR systems use RNA-guided nucleases termed CRISPR-associated or “Cas” endonucleases (e.g., Cas9 or Cpfl) to cleave foreign DNA. In a typical CRISPR/Cas system, an endonuclease is directed to a target nucleotide sequence (e.g., a site in the genome that is to be sequence-edited) by sequence-specific, non-coding “guide RNAs” that target single- or double-stranded DNA sequences. Three classes (I-III) of CRISPR systems have been identified. The class II CRISPR systems use a single Cas endonuclease (rather than multiple Cas proteins). One class II CRISPR system includes a type II Cas endonuclease such as Cas9, a CRISPR RNA (“crRNA”), and a trans-activating crRNA (“tracrRNA”). The crRNA contains a “guide RNA”, typically about 20-nucleotide RNA sequence that corresponds to a target DNA sequence. The crRNA also contains a region that binds to the tracrRNA to form a partially double-stranded structure which is cleaved by RNase III, resulting in a crRNA/tracrRNA hybrid. The crRNA/tracrRNA hybrid then directs the Cas9 endonuclease to recognize and cleave the target DNA sequence. The target DNA sequence must generally be adjacent to a “protospacer adjacent motif (“PAM”) that is specific for a given Cas endonuclease; however, PAM sequences appear throughout a given genome. CRISPR endonucleases identified from various prokaryotic species have unique PAM sequence requirements; examples of PAM sequences include 5′-NGG (Streptococcus pyogenes), 5′-NNAGAA (Streptococcus thermophilus CRISPR1), 5′-NGGNG (Streptococcus thermophilus CRISPR3), and 5′-NNNGATT (Neisseria meningiditis). Some endonucleases, e.g., Cas9 endonucleases, are associated with G-rich PAM sites, e.g., 5′-NGG, and perform blunt-end cleaving of the target DNA at a location 3 nucleotides upstream from (5′ from) the PAM site. Another class II CRISPR system includes the type V endonuclease Cpfl, which is smaller than Cas9; examples include AsCpfl (from Acidaminococcus sp.) and LbCpfl (from Lachnospiraceae sp.). Cpf 1-associated CRISPR arrays are processed into mature crRNAs without the requirement of a tracrRNA; in other words a Cpfl system requires only the Cpfl nuclease and a crRNA to cleave the target DNA sequence. Cpfl endonucleases, are associated with T-rich PAM sites, e.g., 5′-TTN. Cpfl can also recognize a 5′-CTA PAM motif. Cpfl cleaves the target DNA by introducing an offset or staggered double-strand break with a 4- or 5-nucleotide 5′ overhang, for example, cleaving a target DNA with a 5-nucleotide offset or staggered cut located 18 nucleotides downstream from (3′ from) from the PAM site on the coding strand and 23 nucleotides downstream from the PAM site on the complimentary strand; the 5-nucleotide overhang that results from such offset cleavage allows more precise genome editing by DNA insertion by homologous recombination than by insertion at blunt-end cleaved DNA. See, e.g., Zetsche et al. (2015) Cell, 163:759-771.

A variety of CRISPR associated (Cas) genes or proteins can be used in the present invention and the choice of Cas protein will depend upon the particular conditions of the method.

Specific examples of Cas proteins include class II systems including Cas1, Cas2, Cas3, Cas4, Cas5, Cas6, Cas7, Cas8, Cas9, Cas10, Cpfl, C2C1, or C2C3. In some embodiments, a Cas protein, e.g., a Cas9 protein, may be from any of a variety of prokaryotic species. In some embodiments a particular Cas protein, e.g., a particular Cas9 protein, is selected to recognize a particular protospacer-adjacent motif (PAM) sequence. In some embodiments, the site-specific targeting moiety includes a sequence targeting polypeptide, such as an enzyme, e.g., Cas9. In certain embodiments a Cas protein, e.g., a Cas9 protein, may be obtained from a bacteria or archaea or synthesized using known methods. In certain embodiments, a Cas protein may be from a gram positive bacteria or a gram negative bacteria. In certain embodiments, a Cas protein may be from a Streptococcus, (e.g., a S. pyogenes, a S. thermophilus) a Crptococcus, a Corynebacterium, a Haemophilus, a Eubacterium, a Pasteurella, a Prevotella, a Veillonella, or a Marinobacter. In some embodiments nucleic acids encoding two or more different Cas proteins, or two or more Cas proteins, may be introduced into a cell, zygote, embryo, or animal, e.g., to allow for recognition and modification of sites comprising the same, similar or different PAM motifs. In some embodiments, the Cas protein is modified to deactivate the nuclease, e.g., nuclease-deficient Cas9, and to recruit transcription activators or repressors, e.g., the co-subunit of the E. coli Pol, VP64, the activation domain of p65, KRAB, or SID4X, to induce epigenetic modifications, e.g., histone acetyltransferase, histone methyltransferase and demethylase, DNA methyltransferase and enzyme with a role in DNA demethylation (e.g., the TET family enzymes catalyze oxidation of 5-methylcytosine to 5-hydroxymethylcytosine and higher oxidative derivatives).

For the purposes of gene editing, CRISPR arrays can be designed to contain one or multiple guide RNA sequences corresponding to a desired target DNA sequence; see, for example, Cong et al. (2013) Science, 339:819-823; Ran et al. (2013) Nature Protocols, 8:2281-2308. At least about 16 or 17 nucleotides of gRNA sequence are required by Cas9 for DNA cleavage to occur; for Cpfl at least about 16 nucleotides of gRNA sequence is needed to achieve detectable DNA cleavage.

Whereas wild-type Cas9 generates double-strand breaks (DSBs) at specific DNA sequences targeted by a gRNA, a number of CRISPR endonucleases having modified functionalities are available, for example: a “nickase” version of Cas9 generates only a single-strand break; a catalytically inactive Cas9 (“dCas9”) does not cut the target DNA but interferes with transcription by steric hindrance. dCas9 can further be fused with a heterologous effector to repress (CRISPRi) or activate (CRISPRa) expression of a target gene. For example, Cas9 can be fused to a transcriptional silencer (e.g., a KRAB domain) or a transcriptional activator (e.g., a dCas9-VP64 fusion). A catalytically inactive Cas9 (dCas9) fused to Fokl nuclease (“dCas9-FokI”) can be used to generate DSBs at target sequences homologous to two gRNAs. See, e.g., the numerous CRISPR/Cas9 plasmids disclosed in and publicly available from the Addgene repository (Addgene, 75 Sidney St., Suite 550A, Cambridge, Mass. 02139; addgene.org/crispr). A “double nickase” Cas9 that introduces two separate double-strand breaks, each directed by a separate guide RNA, is described as achieving more accurate genome editing by Ran et al. (2013) Cell, 154: 1380-1389. CRISPR technology for editing the genes of eukaryotes is disclosed in US Patent Application Publications 2016/0138008A1 and US2015/0344912A1, and in U.S. Pat. Nos. 8,697,359, 8,771,945, 8,945,839, 8,999,641, 8,993,233, 8,895,308, 8,865,406, 8,889,418, 8,871,445, 8,889,356, 8,932,814, 8,795,965, and 8,906,616. Cpfl endonuclease and corresponding guide RNAs and PAM sites are disclosed in US Patent Application Publication 2016/0208243 A1.

In some embodiments, an effector comprises one or more components of a CRISPR system described hereinabove.

In some embodiments, suitable effectors for use in the agents, compositions, and methods of the invention include, for example, nucleases, physical blockers, epigenetic recruiters, e.g., a transcriptional enhancer or a transcriptional repressor, and epigenetic CpG modifiers, e.g., a DNA methylase, a DNA demethylase, a histone modifying agent, a histone transacetylase, or a histone deacetylase, and combinations of any of the foregoing.

Suitable effectors include a polypeptide or its variant. The term “variant,” as used herein, refers to a polypeptide that is derived by incorporation of one or more amino acid insertions, substitutions, or deletions in a precursor polypeptide (e.g., “parent” polypeptide). In certain embodiments, a variant polypeptide has at least about 85% amino acid sequence identity, e.g., about 90%, about 91%, about 92%, about 93%, about 94%, about 95%, about 96%, about 97%, about 98%, about 99%, or about 100%, amino acid sequence identity to the entire amino acid sequence of a parent polypeptide.

The term “sequence identity,” as used herein, refers to a comparison between pairs of nucleic acid or amino acid molecules, i.e., the relatedness between two amino acid sequences or between two nucleotide sequences. In general, the sequences are aligned so that the highest order match is obtained. Methods for determining sequence identity are known and can be determined by commercially available computer programs that can calculate the percentage of identity between two or more sequences. A typical example of such a computer program is CLUSTAL.

Exemplary effectors include ubiquitin, bicyclic peptides as ubiquitin ligase inhibitors, transcription factors, DNA and protein modification enzymes such as topoisomerases, topoisomerase inhibitors such as topotecan, DNA methyltransferases such as the DNMT family (e.g., DNMT3a, DNMT3b, DNMTL), protein methyltransferases (e.g., viral lysine methyltransferase (vSET), protein-lysine N-methyltransferase (SMYD2), deaminases (e.g., FOXP3 EC, UG1), histone methyltransferases such as enhancer of zeste homolog 2 (EZH2), PRMT1, histone-lysine-N-methyltransferase (Setdbl), histone methyltransferase (SET2), euchromatic histone-lysine N-methyltransferase 2 (G9a), histone-lysine N-methyltransferase (SUV39H1), and G9a), histone deacetylase (e.g., HDAC1, HDAC2, HDAC3), enzymes with a role in DNA demethylation (e.g., the TET family enzymes catalyze oxidation of 5-methylcytosine to 5-hydroxymethylcytosine and higher oxidative derivatives), protein demethylases such as KDMIA and lysine-specific histone demethylase 1 (LSD1), helicases such as DHX9, acetyltransferases, deacetylases (e.g., sirtuin 1, 2, 3, 4, 5, 6, or 7), kinases, phosphatases, DNA-intercalating agents such as ethidium bromide, sybr green, and proflavine, efflux pump inhibitors such as peptidomimetics like phenylalanine arginyl-naphthylamide or quinoline derivatives, nuclear receptor activators and inhibitors, proteasome inhibitors, competitive inhibitors for enzymes such as those involved in lysosomal storage diseases, zinc finger proteins, TALENs, specific domains from proteins, such as a KRAB domain, a VP64 domain, a p300 domain (e.g., p300 core domain), an MeCP2 domain, an MQ1 domain, a DNMT3a-3L domain, a TET1 domain, and/or TET2 domain, protein synthesis inhibitors, nucleases (e.g., Cpfl, Cas9, zinc finger nuclease), fusions of one or more thereof (e.g., dCas9-DNMT, dCas9-FOXP3 EC, dCas9-UG1, dCas9-VP64, dCas9-p300 core, dCas9-KRAB, dCas9-KRAB-MeCP2, dCas9-MQ1, dCas9-DNMT3a-3L, dCas9-TET1/TET2, and dCas9-MC/MN).

In some embodiments, a suitable nuclease for use in the agent, compositions, and methods of the invention comprises a Cas9 polypeptide, or enzymatically active portion thereof. In one embodiment, the Cas9 polypeptide, or enzymatically active portion thereof, further comprises a catalytically active domain of human exonuclease 1 (hEXO1), e.g., 5′ to 3′ exonuclease activity and/or an RNase H activity. In other embodiments, a suitable nuclease comprises a transcription activator like effector nuclease (TALEN). In yet other embodiments, a suitable nuclease comprises a zinc finger protein.

The term TALEN, as used herein, is broad and includes a monomeric TALEN that can cleave double stranded DNA without assistance from another TALEN. The term TALEN is also used to refer to one or both members of a pair of TALENs that are engineered to work together to cleave DNA at the same site. TALENs that work together may be referred to as a left-TALEN and a right-TALEN, which references the handedness of DNA. See U.S. Ser. No. 12/965,590; U.S. Ser. No. 13/426,991 (U.S. Pat. No. 8,450,471); U.S. Ser. No. 13/427,040 (U.S. Pat. No. 8,440,431); U.S. Ser. No. 13/427,137 (U.S. Pat. No. 8,440,432); and U.S. Ser. No. 13/738,381, all of which are incorporated by reference herein in their entirety.

TAL effectors (TALE) are proteins secreted by Xanthomonas bacteria. The DNA binding domain contains a highly conserved 33-34 amino acid sequence with the exception of the 12th and 13th amino acids. These two locations are highly variable (Repeat Variable Diresidue (RVD)) and show a strong correlation with specific nucleotide recognition. This simple relationship between amino acid sequence and DNA recognition has allowed for the engineering of specific DNA binding domains by selecting a combination of repeat segments containing the appropriate RVDs.

The non-specific DNA cleavage domain from the end of the Fold endonuclease can be used to construct hybrid nucleases that are active in a yeast assay. These reagents are also active in plant cells and in animal cells. Initial TALEN studies used the wild-type FokI cleavage domain, but some subsequent TALEN studies also used FokI cleavage domain variants with mutations designed to improve cleavage specificity and cleavage activity. The FokI domain functions as a dimer, requiring two constructs with unique DNA binding domains for sites in the target genome with proper orientation and spacing. Both the number of amino acid residues between the TALE DNA binding domain and the FokI cleavage domain and the number of bases between the two individual TALE binding sites are parameters for achieving high levels of activity. The number of amino acid residues between the TALE DNA binding domain and the FokI cleavage domain may be modified by introduction of a spacer (distinct from the spacer sequence) between the plurality of TAL effector repeat sequences and the FokI endonuclease domain. The spacer sequence may be 12 to 30 nucleotides, e.g., 12-15, 12-20, 20-25, or 15-30 nucleotides.

The relationship between amino acid sequence and DNA recognition of the TALE binding domain allows for designable proteins. In this case artificial gene synthesis is problematic because of improper annealing of the repetitive sequence found in the TALE binding domain. One solution to this is to use a publicly available software program (DNAWorks) to calculate oligonucleotides suitable for assembly in a two step PCR; oligonucleotide assembly followed by whole gene amplification. A number of modular assembly schemes for generating engineered TALE constructs have also been reported. Both methods offer a systematic approach to engineering DNA binding domains that is conceptually similar to the modular assembly method for generating zinc finger DNA recognition domains.

Once the TALEN genes have been assembled they are inserted into plasmids; the plasmids are then used to transfect the target cell where the gene products are expressed and enter the nucleus to access the genome. TALENs can be used to edit genomes by inducing double-strand breaks (DSB), which cells respond to with repair mechanisms. In this manner, they can be used to correct mutations in the genome which, for example, cause disease.

As used herein, a “zinc finger polypeptide” or “zinc finger protein” is a protein that binds to DNA, RNA and/or protein, in a sequence-specific manner, by virtue of a metal stabilized domain known as a zinc finger. Zinc finger proteins are nucleases having a DNA cleavage domain and a DNA binding zinc finger domain Zinc finger polypeptides may be made by fusing the nonspecific DNA. cleavage domain of an endonuclease with site-specific DNA binding zinc finger domains. Such nucleases are powerful tools for gene editing and can be assembled to induce double strand breaks (DSBs) site-specifically into genomic DNA. ZFNs allow specific gene disruption as during DNA repair, the targeted genes can be disrupted via mutagenic non-homologous end joint (NHEJ) or modified via homologous recombination (HR) if a closely related DNA template is supplied.

Zinc finger nucleases are chimeric enzymes made by fusing the nonspecific DNA. cleavage domain of the endonuclease FokI with site-specific DNA binding zinc finger domains. Due to the flexible nature of zinc finger proteins (ZFPs), ZFNs can be assembled that induce double strand breaks (DSBs) site-specifically into genomic DNA. ZFNs allow specific gene disruption as during DNA repair, the targeted genes can be disrupted via mutagenic non-homologous end joint (NHEJ) or modified via homologous recombination (HR) if a closely related DNA template is supplied.

In some embodiments, a suitable physical blocker for use in the agent, compositions, and methods of the invention comprises a gRNA, antisense DNA, or triplex forming oligonucleotide (which may target an expression control unit) steric block a transcriptional control element or anchoring sequence. The gRNA recognizes specific DNA sequences and further includes sequences that interfere with, e.g., a conjunction nucleating molecule sequence to act as a steric blocker. In some embodiments, the gRNA is combined with one or more peptides, e.g., S-adenosyl methionine (SAM), that acts as a steric presence. In other embodiments, a physical blocker comprises an enzymatically inactive Cas9 polypeptide, or fragment thereof (e.g., dCas9).

In one embodiment, an epigenetic recruiter activates or enhances transcription of a target gene. In some embodiments, a suitable epigenetic recruiter for use in the agent, compositions, and methods of the invention comprises a VP64 domain or a p300 core domain.

In one embodiment, an epigenetic recruiter silences or represses transcription of a target gene. In some embodiments, a suitable epigenetic recruiter for use in the agent, compositions, and methods of the invention comprises a KRAB domain, or an MeCP2 domain.

In one embodiment, a suitable epigenetic recruiter for use in the agent, compositions, and methods of the invention comprises dCas9-VP64 fusion, a dCas9-p300 core fusion, a dCas9-KRAB fusion, or a dCas9-KRAB-MeCP2 fusion.

As used herein, “VP64” is a transcriptional activator composed of four tandem copies of VP16 (Herpes Simplex Viral Protein 16, amino acids 437-447*: DALDDFDLDML (SEQ ID NO: 95)) connected with glycine-serine (GS) linkers. In one embodiment, the VP64 further comprises the transcription factors p65 and Rta at the C terminus. The VP64 that comprises p65 and Rta is sometimes referred to as “VPR,” or “VP64-p65-Rta.” The VP64-p65-Rta, or VPR, was created by adding the transcription factors p65 and Rta to the Vp64 at the C terminus. Therefore, all three transcription factors can be targeted to the same gene. The use of three transcription factors, as opposed to solely Vp64, can result in increased expression of targeted genes. The GenBank Accession number of VP64 is ADD60007.1, the GenBank Accession number of p65 is NP_001138610.1, and the GenBank Accession number of Rta is AAA66528.1.

An exemplary amino acid sequence of a VPR is as follows:

(SEQ ID NO.: 64)
DALDDFDLDMLGSDALDDFDLDMLGSDALDDFDLDMLGSDALDDFDLDML
SGGPKKKRKVGSQYLPDTDDRHRIEEKRKRTYETFKSIMKKSPFSGPTDP
RPPPRRIAVPSRSSASVPKPAPQPYPFTSSLSTINYDEFPTMVFPSGQIS
QASALAPAPPQVLPQAPAPAPAPAMVSALAQAPAPVPVLAPGPPQAVAPP
APKPTQAGEGTLSEALLQLQFDDEDLGALLGNSTDPAVFTDLASVDNSEF
QQLLNQGIPVAPHTTEPMLMEYPEAITRLVTGAQRPPDPAPAPLGAPGLP
NGLLSGDEDFSSIADMDFSALLGSGSGSRDSREGMFLPKPEAGSAISDVF
EGREVCQPKRLRPFHPPGSPWANRPLPASLAPTPTGPVHEPVGSLTPAPV
PQPLDPAPAVTPEASHLLEDPDEETSQAVKALREMADTVIPQKEEAAICG
QMDLSHPPPRGHLDELTTTLESMTEDLNLDSPLTPELNEILDTFLNDECL
LHAMHISTGLSIFDTSLF.

As used herein, “p300 core domain” refers to the catalytic core of the human acetyltransferase p300. The GenBank Accession number for the protein comprising p300 is NP_001420.2.

An exemplary amino acid sequence of a p300 is as follows:

(SEQ ID NO.: 65)
IFKPEELRQALMPTLEALYRQDPESLPFRQPVDPQLLGIPDYFDIVKSPM
DLSTIKRKLDTGQYQEPWQYVDDIWLMFNNAWLYNRKTSRVYKYCSKLSE
VFEQEIDPVMQSLGYCCGRKLEFSPQTLCCYGKQLCTIPRDATYYSYQNR
YHFCEKCFNEIQGESVSLGDDPSQPQTTINKEQFSKRKNDTLDPELFVEC
TECGRKMHQICVLHHEIIWPAGFVCDGCLKKSARTRKENKFSAKRLPSTR
LGTFLENRVNDFLRRQNHPESGEVTVRVVHASDKTVEVKPGMKARFVDSG
EMAESFPYRTKALFAFEEIDGVDLCFFGMHVQEYGSDCPPPNQRRVYISY
LDSVHFFRPKCLRTAVYHEILIGYLEYVKKLGYTTGHIWACPPSEGDDYI
FHCHPPDQKIPKPKRLQEWYKKMLDKAVSERIVHDYKDIFKQATEDRLTS
AKELPYFEGDFWPNVLEESIKELEQEEEERKREENTSNESTDVTKGDSKN
AKKKNNKKTSKNKSSLSRGNKKKPGMPNVSNDLSQKLYATMEKHKEVFFV
IRLIAGPAANSLPPIVDPDPLIPCDLMDGRDAFLTLARDKHLEFSSLRRA
QWSTMCMLVELHTQSQD.

As used herein, “KRAB” refers to a Krüppel associated box (KRAB) transcriptional repression domain present in human zinc finger protein-based transcription factors (KRAB zinc finger proteins).

As used herein, MeCp2″ refers to methyl CpG binding protein 2 which represses transcription, e.g., by binding to a promoter comprising methylated DNA.

In one embodiment, an epigenetic CpG modifier methylates DNA and inactivates or represses transcription. In some embodiments, a suitable epigenetic CpG modifier for use in the agent, compositions, and methods of the invention comprises a MQ1 domain or a DNMT3a-3L domain.

In one embodiment, an epigenetic CpG modifier demethylates DNA and activates or stimulates transcription. In some embodiments, a suitable epigenetic recruiter for use in the agent, compositions, and methods of the invention comprises a TET1 or TET2 domain.

As used herein “MQ1” refers to a prokaryotic DNA methyltransferase.

As used herein “DNMT3a-3L” refers to a fusion of a DNA methyltransferase, Dnmt3a and a Dnmt3L which is catalytically inactive, but directly interacts with the catalytic domains of Dnmt3a.

As used herein “TET1” refers to “ten-eleven translocation methylcytosine dioxygenase 1,” a member of the TET family of enzymes, encoded by the TET1 gene. TET1 is a dioxygenase that catalyzes the conversion of the modified DNA base 5-methylcytosine (5-mC) to 5-hydroxymethylcytosine (5-hmC) by oxidation of 5-mC in an iron and alpha-ketoglutarate dependent manner, the initial step of active DNA demethylation in mammals. Methylation at the C5 position of cytosine bases is an epigenetic modification of the mammalian genome which plays an important role in transcriptional regulation. In addition to its role in DNA demethylation, plays a more general role in chromatin regulation. Preferentially binds to CpG-rich sequences at promoters of both transcriptionally active and Polycomb-repressed genes. Involved in the recruitment of the O-GlcNAc transferase OGT to CpG-rich transcription start sites of active genes, thereby promoting histone H2B GlcNAcylation by OGT. Exemplary TET 1 nucleotide and amino acid sequence can be found at GenBank Accession Nos.: NM_030625.3, NP_085128.2 As used herein, “TET2” refers to “ten-eleven translocation 2 (TET2),” a member of the TET family of enzymes, encoded by the TET1 gene. Similarly to TET1, TET2 is a dioxygenase that catalyzes the conversion of the modified genomic base 5-methylcytosine (5mC) into 5-hydroxymethylcytosine (5hmC) and plays a key role in active DNA demethylation. TET2 a preference for 5-hydroxymethylcytosine in CpG motifs. TET2 also mediates subsequent conversion of 5hmC into 5-formylcytosine (5fC), and conversion of 5fC to 5-carboxylcytosine (5caC). The conversion of 5mC into 5hmC, 5fC and 5caC probably constitutes the first step in cytosine demethylation. Methylation at the C5 position of cytosine bases is an epigenetic modification of the mammalian genome which plays an important role in transcriptional regulation. In addition to its role in DNA demethylation, also involved in the recruitment of the O-GlcNAc transferase OGT to CpG-rich transcription start sites of active genes, thereby promoting histone H2B GlcNAcylation by OGT. Exemplary nucleotide and amino acid sequence can be found at Genbank Accession No.: NM_001127208.2, NP_001120680.1

In some embodiments, a suitable epigenetic recruiter for use in the agent, compositions, and methods of the invention comprises a MQ1 domain, a DNMT3a-3L, a TET1 or TET2 domain. In one embodiment, a suitable epigenetic recruiter for use in the agent, compositions, and methods of the invention comprises a dCas9-MQ1 fusion, a dCas9-DNMT3a-3L fusion, or a dCas9-TET1 fusion or −dCase9-TET2 fusion.

### III. Delivery of a Site-Specific FOXP3 Disrupting Agent of the Invention and Compositions Comprising a Site-Specific a FOXP3 Disrupting Agents of the Invention

The delivery of the disrupting agents of the invention to a cell e.g., a cell within a subject, such as a human subject (e.g., a subject in need thereof, such as a subject having a FOXP3-associated disorder, e.g., an autoimmune disease, such as IPEX syndrome) may be achieved in a number of different ways. For example, delivery may be performed by contacting a cell with a disrupting agent of the invention either in vitro, ex vivo, or in vivo. In vivo delivery may be performed directly by administering a composition, such as a lipid composition, comprising a disrupting agent to a subject. Alternatively, in vivo delivery may be performed indirectly by administering one or more vectors that encode and direct the expression of the disrupting agent in a cell of a subject. These alternatives are discussed further below. In vitro introduction into a cell includes methods known in the art such as electroporation and lipofection. Further approaches are described herein below and/or are known in the art.

In some embodiments, the disrupting agent comprises a nucleic acid molecule encoding a fusion protein, the fusion protein comprising a site-specific FOXP3 targeting moiety, such as a polynucleotide encoding a DNA-binding domain of a Transcription activator-like effector (TALE) polypeptide or a zinc finger (ZNF) polypeptide, or fragment thereof, that specifically targets and binds to the FOXP3 expression control region and an effector molecule, such as a VPR.

In other embodiments, the disrupting agent comprises a guide RNA and an mRNA encoding an effector molecule. The ratio of guide RNA to mRNA may be about 100:1 to about 1:100 (wt:wt).

In general, any method of delivery of a site-specific FOXP3 disrupting agent of the invention (in vitro, ex vivo, or in vivo) may be adapted for use with the disrupting agents of the invention (see e.g., Akhtar S. and Julian R L., (1992) Trends Cell. Biol. 2(5):139-144 and WO94/02595, which are incorporated herein by reference in their entireties). For in vivo delivery, factors to be considered for delivering a site-specific FOXP3 disrupting agent of the invention include, for example, biological stability of the disrupting agent, prevention of non-specific effects, and accumulation of the disrupting agent in the target tissue. The non-specific effects of a disrupting agent can be minimized by local administration, for example, by direct injection or implantation into a tissue or topically administering a composition comprising the disrupting agent. Local administration to a treatment site maximizes local concentration of the disrupting agent, limits the exposure of the disrupting agent to systemic tissues that can otherwise be harmed by the disrupting agent or that can degrade the disrupting agent, and permits a lower total dose of the disrupting agent to be administered.

For administering a site-specific FOXP3 disrupting agent systemically for the treatment of a disease, such as a FOXP3-associate disease, the disrupting agent, e.g., a disrupting agent comprising a site-specific targeting moiety comprising a nucleic acid molecule, can be modified or alternatively delivered using a drug delivery system; both methods act to prevent the rapid degradation of a site-specific targeting moiety comprising a nucleic acid molecule by endo- and exo-nucleases in vivo. Modification of a disrupting agent comprising a site-specific targeting moiety comprising a nucleic acid molecule or a pharmaceutical carrier also permits targeting of the disrupting agent to a target tissue and avoidance of undesirable off-target effects. For example, a disrupting agent of the invention may be modified by chemical conjugation to lipophilic groups such as cholesterol to enhance cellular uptake and prevent degradation.

Alternatively, a disrupting agent of the invention may be delivered using a drug delivery system such as a nanoparticle, a dendrimer, a polymer, a liposome, or a cationic delivery system. Positively charged cationic delivery systems facilitate binding of disrupting agent (e.g., negatively charged molecule) and also enhance interactions at the negatively charged cell membrane to permit efficient uptake of a disrupting agent by the cell. Cationic lipids, dendrimers, or polymers can either be bound to a disrupting agent, or induced to form a vesicle or micelle (see e.g., Kim S H. et al., (2008) Journal of Controlled Release 129(2):107-116) that encases the disrupting agent. The formation of vesicles or micelles further prevents degradation of the disrupting agent when administered systemically. Methods for making and administering cationic complexes are well within the abilities of one skilled in the art (see e.g., Sorensen, D R., et al. (2003) J. Mol. Biol 327:761-766; Verma, U N. et al., (2003) Clin. Cancer Res. 9:1291-1300; Arnold, A S et al. (2007) J. Hypertens. 25:197-205, which are incorporated herein by reference in their entirety). Some non-limiting examples of drug delivery systems useful for systemic delivery of a disrupting agent of the invention include DOTAP (Sorensen, D R., et al (2003), supra; Verma, U N. et al., (2003), supra), Oligofectamine, “solid nucleic acid lipid particles” (Zimmermann, T S. et al., (2006) Nature 441:111-114), cardiolipin (Chien, P Y. et al., (2005) Cancer Gene Ther. 12:321-328; Pal, A. et al., (2005) Int J. Oncol. 26:1087-1091), polyethyleneimine (Bonnet M E. et al., (2008) Pharm. Res. August 16 Epub ahead of print; Aigner, A. (2006) J. Biomed. Biotechnol. 71659), Arg-Gly-Asp (RGD) peptides (Liu, S. (2006) Mol. Pharm. 3:472-487), and polyamidoamines (Tomalia, D A. et al., (2007) Biochem. Soc. Trans. 35:61-67; Yoo, H. et al., (1999) Pharm. Res. 16:1799-1804). In some embodiments, a disrupting agent (e.g., gRNA, or mRNA) forms a complex with cyclodextrin for systemic administration. Methods for administration and pharmaceutical compositions comprising cyclodextrins may be found in U.S. Pat. No. 7,427,605, the entire contents of which are incorporated herein by reference.

The disrupting agents of the invention may be incorporated into pharmaceutical compositions suitable for administration. Such compositions typically include one or more species of disrupting agent and a pharmaceutically acceptable carrier. As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

The pharmaceutical compositions of the present invention may be administered in a number of ways depending upon whether local or systemic treatment is desired and upon the area to be treated. Administration may be topical (including ophthalmic, vaginal, rectal, intranasal, transdermal), oral, or parenteral. Parenteral administration includes intravenous drip, subcutaneous, intraperitoneal or intramuscular injection, or intrathecal or intraventricular administration.

The route and site of administration may be chosen to enhance delivery or targeting of the disrupting agent comprising a site-specific targeting moiety to a particular location. For example, to target liver cells, intravenous injection may be used. Lung cells may be targeted by administering the disrupting agent in aerosol form. Jejunum cells may be targeted by anal administration.

Formulations for topical administration may include transdermal patches, ointments, lotions, creams, gels, drops, suppositories, sprays, liquids, and powders. Conventional pharmaceutical carriers, aqueous, powder or oily bases, thickeners and the like may be necessary or desirable. Coated condoms, gloves and the like may also be useful.

Compositions for oral administration include powders or granules, suspensions or solutions in water, syrups, elixirs or non-aqueous media, tablets, capsules, lozenges, or troches. In the case of tablets, carriers that can be used include lactose, sodium citrate and salts of phosphoric acid. Various disintegrants such as starch, and lubricating agents such as magnesium stearate, sodium lauryl sulfate and talc, are commonly used in tablets. For oral administration in capsule form, useful diluents are lactose and high molecular weight polyethylene glycols. When aqueous suspensions are required for oral use, the nucleic acid compositions can be combined with emulsifying and suspending agents. If desired, certain sweetening or flavoring agents can be added.

Compositions for intravenous administration may include sterile aqueous solutions which may also contain buffers, diluents, and other suitable additives.

Formulations for parenteral administration may include sterile aqueous solutions which may also contain buffers, diluents, and other suitable additives. For intravenous use, the total concentration of solutes may be controlled to render the preparation isotonic.

In one embodiment, the administration of a disrupting agent composition of the invention is parenteral, e.g., intravenous (e.g., as a bolus or as a diffusible infusion), intradermal, intraperitoneal, intramuscular, intrathecal, intraventricular, intracranial, subcutaneous, transmucosal, buccal, sublingual, endoscopic, rectal, oral, vaginal, topical, pulmonary, intranasal, urethral, or ocular. Administration can be provided by the subject or by another person, e.g., a health care provider. The composition may be provided in measured doses or in a dispenser which delivers a metered dose. Selected modes of delivery are discussed in more detail below.

In certain embodiments, the disrupting agents of the invention are polynucleotides, such as mRNAs, and are formulated in lipid nanoparticles (LNPs).

A. Compositions Comprising a Site-Specific a FOXP3 Disrupting Agent of the Invention

The site-specific FOXP3 disrupting agents of the invention may be formulated into compositions, such as pharmaceutical compositions, using one or more excipients to: (1) increase stability; (2) increase cell transfection; (3) permit sustained or delayed release (e.g., from a depot formulation); (4) alter the biodistribution (e.g., target the disrupting agent to specific tissues or cell types); (5) increase the translation of an encoded protein in vivo; and/or (6) alter the release profile of an encoded protein in vivo. In addition to traditional excipients, such as any and all solvents, dispersion media, diluents, or other liquid vehicles, dispersion or suspension aids, surface active agents, isotonic agents, thickening or emulsifying agents, preservatives, excipients for use in the compositions of the invention may include, without limitation, lipidoids, liposomes, lipid nanoparticles, polymers, lipoplexes, core-shell nanoparticles, peptides, proteins, cells transfected with nucleic acid molecules, modified nucleic acid molecules, or RNA (e.g., for transplantation into a subject), hyaluronidase, nanoparticle mimics and combinations thereof. Accordingly, the pharmaceutical compositions of the invention can include one or more excipients, each in an amount that together increases the stability of the disrupting agent, increases cell transfection by the disrupting agent, increases the expression of modified nucleic acid, or mRNA encoded protein, and/or alters the release profile of a disrupting agent. Further, the disrupting agents of the present invention may be formulated using self-assembled nucleic acid nanoparticles (see, e.g., U.S. Patent Publication No. 2016/0038612A1, which is incorporated herein by reference in its entirety.

i. Lipidoid

The synthesis of lipidoids has been extensively described and formulations containing these compounds are particularly suited for delivery of a disrupting agent of the invention, such as a disrupting agent comprising a site-specific FOXP3 targeting moiety comprising a nucleic acid molecule, e.g., comprising modified nucleic acid molecules or mRNA (see Mahon et al., Bioconjug Chem. 2010 21:1448-1454; Schroeder et al., J Intern Med. 2010 267:9-21; Akinc et al., Nat Biotechnol. 2008 26:561-569; Love et al., Proc Natl Acad Sci USA. 2010 107: 1864-1869; Siegwart et al., Proc Natl Acad Sci USA. 2011108:12996-3001; the contents of all of which are incorporated herein in their entireties).

For example, lipidoids have been used to effectively deliver double stranded small interfering RNA molecules, single stranded nucleic acid molecules, modified nucleic acid molecules or modified mRNA. (See, e.g., US Patent Publication 2016/0038612A1). Complexes, micelles, liposomes or particles can be prepared containing these lipidoids and, therefore, provide effective delivery of a site-specific FOXP3 targeting moiety comprising a nucleic acid molecule, as judged by the production of an encoded protein, following the administration of a lipidoid formulation, e.g., via localized and/or systemic administration. Lipidoid complexes of can be administered by various means including, but not limited to, intravenous, intramuscular, intradermal, intraperitoneal or subcutaneous routes.

In vivo delivery of a site-specific FOXP3 targeting moiety comprising, e.g., a nucleic acid molecule, may be affected by many parameters, including, but not limited to, the formulation composition, nature of particle PEGylation, degree of loading, polynucleotide to lipid ratio, and biophysical parameters such as, but not limited to, particle size (Akinc et al., Mol Ther. 2009 17:872-879; herein incorporated by reference in its entirety). As an example, small changes in the anchor chain length of poly(ethylene glycol) (PEG) lipids may result in significant effects on in vivo efficacy. Formulations with different lipidoids, including, but not limited to penta[3-(1-laurylaminopropiony I)]-triethy lenetetramine hydrochloride (TETA-5LAP; aka 98NI2-5, see Murugaiah et al., Analytical Biochemistry, 401:61 (2010); the contents of which are herein incorporated by reference in its entirety), C12-200 (including derivatives and variants), and MD1, may be used.

In one embodiment, a disrupting agent comprising a site-specific FOXP3 targeting moiety comprising, e.g., a nucleic acid molecule, is formulated with a lipidoid for systemic intravenous administration to target cells of the liver. For example, a final optimized intravenous formulation comprising a disrupting agent comprising a site-specific FOXP3 targeting moiety comprising a nucleic acid molecule, and a lipid molar composition of 42% 98NI2-5, 48% cholesterol, and 10% PEG-lipid with a final weight ratio of about 7.5 to 1 total lipid to nucleic acid molecule, and a C14 alkyl chain length on the PEG lipid, with a mean particle size of roughly 50-60 nm, can result in the distribution of the formulation to be greater than 90% to the liver (see, Akinc et al., Mol Ther. 2009 17:872-879; the contents of which is herein incorporated by reference in its entirety). In another example, an intravenous formulation using a C12-200 lipidoid (see, e.g., PCT Publication No. WO 2010/129709, which is herein incorporated by reference in their entirety) having a molar ratio of 50/10/38.5/1.5 of C12-200/disteroylphosphatidyl choline/cholesterol/PEG-DMG, with a weight ratio of 7 to 1 total lipid to nucleic acid molecule, and a mean particle size of 80 nm may be used to deliver a disrupting agent comprising a site-specific FOXP3 targeting moiety comprising a nucleic acid molecule, to hepatocytes (see, Love et al., Proc Natl Acad Sci USA. 2010 107:1864-1869; the contents of which are herein incorporated by reference in its entirety). In another embodiment, an MD1 lipidoid-containing formulation may be used to effectively deliver a disrupting agent comprising a site-specific FOXP3 targeting moiety comprising a nucleic acid molecule, to hepatocytes in vivo. The characteristics of optimized lipidoid formulations for intramuscular or subcutaneous routes may vary significantly depending on the target cell type and the ability of formulations to diffuse through the extracellular matrix into the blood stream. While a particle size of less than 150 nm may be desired for effective hepatocyte delivery due to the size of the endothelial fenestrae (see, Akinc et al., Mol Ther. 2009 17:872-879; the contents of which are herein incorporated by reference in their entirety), use of lipidoid-formulated nucleic acid molecules to deliver the formulation to other cells types including, but not limited to, endothelial cells, myeloid cells, and muscle cells may not be similarly size-limited. Use of lipidoid formulations to deliver siRNA in vivo to other non-hepatocyte cells such as myeloid cells and endothelium has been reported (see Akinc et al., Nat Biotechnol. 200826:561-569; Leuschner et al., Nat Biotechnol. 2011 29: 1005-101 0; Cho et al. Adv. Funct. Mater. 2009 19:3112-3118; 8th International Judah Folkman Conference, Cambridge, Mass. Oct. 8-9, 2010; the contents of each of which are herein incorporated by reference in their entirety). For delivery to myeloid cells, such as monocytes, lipidoid formulations may have a similar component molar ratio. Different ratios of lipidoids and other components including, but not limited to, disteroylphosphatidyl choline, cholesterol and PEG-DMG, may be used to optimize the formulation for delivery to different cell types including, but not limited to, hepatocytes, myeloid cells, muscle cells, etc. For example, the component molar ratio may include, but is not limited to, 50% CI2-200, 10% disteroylphosphatidyl choline, 38.5% cholesterol, and 1.5% PEG-DMG (see Leuschner et al., Nat Biotechnol 2011 29: 1005-101 0; the contents of which are herein incorporated by reference in their entirety). The use of lipidoid formulations for the localized delivery to cells (such as, but not limited to, adipose cells and muscle cells) via either subcutaneous, intradermal or intramuscular delivery, may not require all of the formulation components desired for systemic delivery and, as such, may comprise only the lipidoid and a disrupting agent comprising a site-specific FOXP3 targeting moiety comprising, e.g., a nucleic acid molecule, as described herein.

Combinations of different lipidoids may be used to improve the efficacy of the formulations by increasing cell transfection and/or increasing the translation of encoded protein contained therein(see Whitehead et al., Mol. Ther. 2011, 19:1688-1694, the contents of which are herein incorporated by reference in their entirety).

In one embodiment, the lipidoid may be prepared from the conjugate addition of alklamines to acrylates. As a non-limiting example, a lipidoid may be prepared by the methods described in PCT Patent Publication No. WO 2014/028487, the contents of which are herein incorporated by reference in its entirety. In one embodiment, the lipidoid may comprise a compound having formula (I), formula (II), formula (III), formula (IV) or formula (V) as described in PCT Patent Publication No. WO 2014/028487, the contents of which are herein incorporated by reference in their entirety. In one embodiment, the lipidoid may be biodegradable.

ii. Liposomes, Lipoplexes, and Lipid Nanoparticles

A disrupting agent of the invention may be formulated using one or more liposomes, lipoplexes, or lipid nanoparticles. In one embodiment, pharmaceutical compositions of the invention include liposomes. Liposomes are artificially-prepared vesicles which are primarily composed of a lipid bilayer and may be used as a delivery vehicle for the administration of nutrients and pharmaceutical formulations. Liposomes may be of different sizes such as, but not limited to, a multilamellar vesicle (MLV) which may be hundreds of nanometers in diameter and may contain a series of concentric bilayers separated by narrow aqueous compartments, a small unicellular vesicle (SUV) which may be smaller than 50 nm in diameter, and a large unilamellar vesicle (LUV) which may be between 50 and 500 nm in diameter. Liposome design may include, but is not limited to, opsonins or ligands in order to improve the attachment of liposomes to unhealthy tissue or to activate events such as, but not limited to, endocytosis. Liposomes may contain a low or a high pH in order to improve the delivery of the pharmaceutical formulations. The formation of liposomes may depend on the physicochemical characteristics such as, but not limited to, the pharmaceutical formulation entrapped and the liposomal ingredients, the nature of the medium in which the lipid vesicles are dispersed, the effective concentration of the entrapped substance and its potential toxicity, any additional processes involved during the application and/or delivery of the vesicles, the optimization size, polydispersity and the shelf-life of the vesicles for the intended application, and the batch-to-batch reproducibility and possibility of large-scale production of safe and efficient liposomal products.

As a non-limiting example, liposomes, such as synthetic membrane vesicles, may be prepared by the methods, apparatus and devices described in U.S. Patent Publication Nos. 2013/0177638, 2013/0177637, 2013/0177636, 201/30177635, 2013/0177634, 2013/0177633, 2013/0183375, 2013/0183373, 2013/0183372 and 2016/0038612) and PCT Patent Publication No WO 2008/042973, the contents of each of which are herein incorporated by reference in their entirety.

In one embodiment, a pharmaceutical composition described herein may include, without limitation, liposomes such as those formed from 1,2-dioleyloxy-N,N-dimethylami-nopropane (DODMA) liposomes, DiLa2 liposomes from Marina Biotech (Bothell, Wash.), 1,2-dilinoleyloxy-3-dimethylaminopropane (DLin-DMA), 2,2-dilinoleyl-4-(2-dimethylaminoethyl)-[1,3]-dioxolane (DLin-KC2-DMA), and MC3 (US20100324120; herein incorporated by reference in its entirety) and liposomes which may deliver small molecule drugs such as, but not limited to, DOXIL® from Janssen Biotech, Inc. (Horsham, Pa.). In one embodiment, a pharmaceutical composition described herein may include, without limitation, liposomes such as those formed from the synthesis of stabilized plasmid-lipid particles (SPLP) or stabilized nucleic acid lipid particle (SNALP) that have been previously described and shown to be suitable for oligonucleotide delivery in vitro and in vivo (see Wheeler et al. Gene Therapy. 1999 6:271-281; Zhang et al. Gene Therapy. 19996:1438-1447; Jeffs et al. Pharm Res. 2005 22:362-372; Morrissey et al., Nat Biotechnol. 2005 2:1002-1007; Zimmermann et al., Nature. 2006 441:111-114; Heyes et al. J Contr Rd. 2005 107:276-287; Semple et al. Nature Biotech. 2010 28:172-176; Judge et al. J Clin Invest. 2009 119:661-673; deFougerolles Hum Gene Ther. 2008 19:125-132; U.S. Patent Publication Nos 2013/0122104, 2013/0303587, and 2016/0038612; the contents of each of which are incorporated herein in their entireties). The original manufacturing method of Wheeler et al. was a detergent dialysis method, which was later improved by Jeffs et al. and is referred to as the spontaneous vesicle formation method. The liposome formulations of the invention may be composed of 3 to 4 lipid components in addition a disrupting agent comprising a site-specific FOXP3 targeting moiety. As an example a liposome of the invention can contain, but is not limited to, 55% cholesterol, 20% disteroylphosphatidyl choline (DSPC), 10% PEG-SDSG, and 15% 1,2-dioleyloxy-N,N-dimethylaminopropane (DODMA), as described by Jeffs et al. As another example, liposome formulations of the invention may contain, but are not limited to, 48% cholesterol, 20% DSPC, 2% PEG-c-DMA, and 30% cationic lipid, where the cationic lipid can be 1,2-distearloxy-N,N-dimethylaminopropane (DSDMA), DODMA, DLin-DMA, or 1,2-dilinolenyloxy-3-dimethylaminopropane (DLenDMA), as described by Heyes et al. In some embodiments, liposome formulations may comprise from about 25.0% cholesterol to about 40.0% cholesterol, from about 30.0% cholesterol to about 45.0% cholesterol, from about 35.0% cholesterol to about 50.0% cholesterol and/or from about 48.5% cholesterol to about 60% cholesterol. In another embodiment, formulations of the invention may comprise a percentage of cholesterol selected from the group consisting of 28.5%, 31.5%, 33.5%, 36.5%, 37.0%, 38.5%, 39.0% and 43.5%. In some embodiments, liposome formulations of the invention may comprise from about 5.0% to about 10.0% DSPC and/or from about 7.0% to about 15.0% DSPC.

In one embodiment, a pharmaceutical composition may include liposomes which may be formed to deliver a disrupting agent of the invention. The disrupting agent comprising a site-specific FOXP3 targeting moiety comprising may be encapsulated by the liposome and/or it may be contained in an aqueous core which may then be encapsulated by the liposome (see, e.g., PCT Patent Publication Nos. WO 2012/031046, WO 2012/031043, WO 2012/030901 and WO 2012/006378 and U.S. Patent Publication Nos. 2013/0189351, 2013/0195969 and 201/30202684, the contents of each of which are herein incorporated by reference in their entirety).

In another embodiment, liposomes for use in the present invention may be formulated for targeted delivery. As a non-limiting example, the liposome may be formulated for targeted delivery to the liver. Such a liposome may include, but is not limited to, a liposome described in U.S. Patent Publication No. 2013/0195967, the contents of which are herein incorporated by reference their its entirety.

In one embodiment, formulations comprising liposomes and a disrupting agent may be administered intramuscularly, intrademrally, or intravenously.

In another embodiment, a lipid formulation of the invention may include at least one cationic lipid, a lipid which enhances transfection and a least one lipid which contains a hydrophilic head group linked to a lipid moiety (International Pub. No. WO2011076807 and U.S. Pub. No. 20110200582; the entire contents of each of which is herein incorporated by reference in their entirety). In another embodiment, a lipid formulation of the invention is a lipid vesicle which may have crosslinks between functionalized lipid bilayers (see U.S. Patent Publication No. 2012/0177724, the contents of which are herein incorporated by reference in their entirety).

In one embodiment, a formulation comprising a disrupting agent is a lipid nanoparticle (LNP) which may comprise at least one lipid. The lipid may be selected from, but is not limited to, DLin-DMA, DLin-K-DMA, 98NI2-5, CI2-200, DLin-MC3-DMA, DLin-KC2-DMA, DODMA, PLGA, PEG, PEG-DMG, PEGylated lipids and amino alcohol lipids. In another aspect, the lipid may be a cationic lipid such as, but not limited to, DLin-DMA, DLin-D-DMA, DLin-MC3-DMA, DLin-KC2-DMA, DODMA and amino alcohol lipids. The amino alcohol cationic lipid may be the lipids described in and/or made by the methods described in U.S. Patent Publication No. 2013/0150625.

In one embodiment, the cationic lipid may be selected from, but not limited to, a cationic lipid described in PCT Publication Nos. WO 2012/040184, WO 2011/153120, WO 2011/149733, WO 2011/090965, WO 2011/043913, WO 2011/022460, WO 2012/061259, WO 2012/054365, WO 2012/044638, WO 2010/080724, WO 2010/21865, WO 2008/103276, WO 2013/086373 and WO 2013/086354, U.S. Pat. Nos. 7,893,302, 7,404,969, 8,283,333, 8,466,122 and 8,569,256, and U.S. Patent Publication Nos. 2010/0036115, 2012/0202871, 2013/0064894, 2013/0129785, 2013/0150625, 2013/0178541, 2013/0225836 and 2014/0039032; the contents of each of which are herein incorporated by reference in their entirety. In another embodiment, the cationic lipid may be selected from, but not limited to, formula A described in PCT Publication Nos. WO 2012/040184, WO 0111/53120, WO 2011/149733, WO 2011/090965, WO 2011/043913, WO 2011/022460, WO 2012/061259, WO 2012/054365, WO 2012/044638 and WO 2013/116126 or U.S. Patent Publication Nos. 2013/0178541 and 2013/0225836; the contents of each of which is herein incorporated by reference in their entirety. In yet another embodiment, the cationic lipid may be selected from, but not limited to, formula CLI-CLXXIX of PCT Publication No. WO 2008/103276, formula CLICLXXIX of U.S. Pat. No. 7,893,302, formula CLICLXXXXII of U.S. Pat. No. 7,404,969 and formula I-VI of us Patent Publication No. 2010/0036115, formula I of U.S. Patent Publication No 2013/0123338; each of which is herein incorporated by reference in their entirety.

In one embodiment, the cationic lipid may be synthesized by methods known in the art and/or as described in PCT Publication Nos. WO 2012/040184 WO 2011/153120, WO 2011/149733, WO 2011/090965: WO 2011/043913, WO 2011/022460, WO 2012/061259, WO 2012/054365, WO 2012/044638, WO 2010/080724, WO 2010/21865, WO 2013/126803, WO 2013/086373, and WO 2013/086354; the contents of each of which are herein incorporated by reference in their entirety.

In one embodiment, the lipids which may be used in the formulations and/or for delivery of the disrupting agents described herein may be a cleavable lipid. As a non-limiting example, a cleavable lipid and/or pharmaceutical compositions comprising cleavable lipids include those described in PCT Patent Publication No. WO 2012/170889, the contents of which are herein incorporated by reference in their entirety. As another non-limiting example, the cleavable lipid may be HGT4001, HGT4002, HGT4003, HGT4004 and/or HGT4005 as described in PCT Patent Publication No. WO 2012/170889, the contents of which are herein incorporated by reference in their entirety.

In one embodiment, polymers which may be used in the formulation and/or delivery of the disrupting agents described herein may include, but is not limited to, poly(ethylene) glycol (PEG), polyethylenimine (PEI), dithiobis(succinimidylpropionate) (DSP), Dimethy 1-3,3′-dithiobispropionimidate (DTBP), poly(ethylene imine) biscarbamate (PEIC), poly(L-lysine) (PLL), histidine modified PLL, poly(N-vinylpyrrohdone) (PVP), poly(propylenimine (PPI), poly(amidoamine) (PAMAM), poly(amido ethylenimine) (SS-PAEI), triehtylenetetramine (TETA), poly(β-aminoester), poly(4-hydroxy-L-proine ester) (PHP), poly(allylamine), poly(α-[4-aminobutyl]-L-glycolic acid (PAGA), Poly(D,L-lactic-coglycolid acid (PLGA), Poly(N-ethyl-4-vinylpyridinium bromide), poly(phosphazene)s (PPZ), poly(phosphoester)s (PPE), poly(phosphoramidate)s (PPA), poly(N-2-hydroxypropylmethacrylamide) (pHPMA), poly(2-(dimethylamino)ethyl methacrylate) (pDMAEMA), poly(2-aminoethyl propylene phosphate) PPE_EA), Chitosan, galactosylated chitosan, N-dodecylated chitosan, histone, collagen and dextran-spermine. In one embodiment, the polymer may be an inert polymer such as, but not limited to, PEG. In one embodiment, the polymer may be a cationic polymer such as, but not limited to, PE1, PLL, TETA, poly(allylamine), Poly(N-ethyl-4-vinylpyridinium bromide), pHPMA and pDMAEMA. In one embodiment, the polymer may be a biodegradable PE1 such as, but not limited to, DSP, DTBP and PEIC. In one embodiment, the polymer may be biodegradable such as, but not limited to, histine modified PLL SSPAEI, poly(β-aminoester), PHP, PAGA, PLGA, PPZ, PPE, PPA and PPE-EA.

In one embodiment, an LNP formulation of the invention may be prepared according to the methods described in PCT Publication Nos. WO 2011/127255 or WO 2008/103276, the contents of each of which are herein incorporated by reference in their entirety. As a non-limiting example, a disrupting agent comprising a site-specific FOXP3 targeting moiety may be encapsulated in an LNP formulation as described in PCT Publication Nos. WO 2011/127255 and/or WO 2008/103276; the contents of each of which are herein incorporated by reference in their entirety. As another non-limiting example, a disrupting agent comprising a site-specific FOXP3 targeting moiety as described herein, may be formulated in a nanoparticle to be delivered by a parenteral route as described in U.S. Patent Publication No. 2012/0207845 and PCT Publication No. WO 2014/008334; the contents of each of which are herein incorporated by reference in their entirety.

In one embodiment, LNP formulations described herein may be administered intramusculary. The LNP formulation may comprise a cationic lipid described herein, such as, but not limited to, DLin-DMA, DLin-KC2-DMA, DLin-MC3-DMA, DODMA and C12-200.

In one embodiment, LNP formulations described herein comprising a disrupting agent as described herein, may be administered intradermally. The LNP formulation may comprise a cationic lipid described herein, such as, but not limited to, DLin-DMA, DLin-KC2-DMA, DLin-MC3-DMA, DODMA and C12-200.

The nanoparticle formulations may comprise conjugate, such as a phosphate conjugate, a polymer conjugates, a conjugate that enhances the delivery of nanoparticle as described in US Patent Publication No. US20160038612 A1.

In one embodiment, the lipid nanoparticle formulation comprises DLin-MC3-DMA as described in US Patent Publication No. US20100324120.

In one embodiment, the lipid nanoparticle comprises a lipid compound, or a pharmaceutically acceptable salt, tautomer or stereoisomer thereof, or a lipid nanoparticle formulation, as described in U.S. patent No.: U.S. Ser. No. 10/723,692B2, US Patent Publication Nos. US20200172472A1, US20200163878A1, US20200046838A1, US20190359556A1, US20190314524A1, US20190274968A1, US20190022247A1, US20180303925A1, US20180185516A1, US20160317676A1, International Patent Publication No.: WO20200146805A1, WO2020081938A1, WO2019089828A1, WO2019036030A1, WO2019036028A1, WO2019036008A1, WO 2018200943A1, WO2018191719A1, WO2018107026A1, WO2018081480A1, the contents of each of which are herein incorporated by reference in their entirety (Acuitas Therapeutics, Inc.).

In one embodiment, the lipid nanoparticle comprises an amino lipid, or a pharmaceutically acceptable salt, tautomer or stereoisomer thereof, or a lipid nanoparticle formulation, described by Tekmira Pharmaceuticals Corp. in U.S. Pat. No. 9,139,554B2, U.S. Pat. No. 9,051,567B2, U.S. Pat. No. 8,883,203B2, US Patent Publication US20110117125A1, the contents of each of which are herein incorporated by reference in their entirety. In one particular example, the compound described in U.S. Pat. No. 9,139,554B2 is DLin-kC2-DMA.

In one embodiment, the lipid nanoparticle comprises an amino lipid, or a pharmaceutically acceptable salt, tautomer or stereoisomer thereof, or a lipid nanoparticle formulation, described by Arbutus Biopharma Corp. in U.S. Ser. No. 10/561,732B2, U.S. Pat. No. 9,938,236B2, U.S. Pat. No. 9,687,550B2, US Patent Publication US20190240354A1, US20170027658A1, WO2020097493A1, WO2020097520A1, WO2020097540A1, WO2020097548A1, the contents of each of which are herein incorporated by reference in their entirety.

Lipid nanoparticles may be engineered to alter the surface properties of particles so the lipid nanoparticles may penetrate the mucosal barrier. Mucus is located on mucosal tissue such as, but not limited to, oral (e.g., the buccal and esophageal membranes and tonsil tissue), ophthalmic, gastrointestinal (e.g., stomach, small intestine, large intestine, colon, rectum), nasal, respiratory (e.g., nasal, pharyngeal, tracheal and bronchial membranes), genital (e.g., vaginal, cervical and urethral membranes). Nanoparticles larger than 10-200 nm which are preferred for higher drug encapsulation efficiency and the ability to provide the sustained delivery of a wide array of drugs have been thought to be too large to rapidly diffuse through mucosal barriers. Mucus is continuously secreted, shed, discarded or digested and recycled so most of the trapped particles may be removed from the mucosla tissue within seconds or within a few hours. Large polymeric nanoparticles (200 nm-500 nm in diameter) which have been coated densely with a low molecular weight polyethylene glycol (PEG) diffused through mucus only 4 to 6-fold lower than the same particles diffusing in water (Lai et al. PNAS 2007 104(5): 1482-487; Lai et al. Adv Drug Deliv Rev. 200961(2): 158-171; the contents of each of which are herein incorporated by reference in their entirety). The transport of nanoparticles may be determined using rates of permeation and/or fluorescent microscopy techniques including, but not limited to, fluorescence recovery after photobleaching (FRAP) and high resolution multiple particle tracking (MPT). As a non-limiting example, compositions which can penetrate a mucosal barrier may be made as described in U.S. Pat. No. 8,241,670 or International Patent Publication No. WO2013110028, the contents of each of which are herein incorporated by reference in their entirety.

In one embodiment, a disrupting agent comprising a site-specific FOXP3 targeting moiety as described herein, is formulated as a lipoplex, such as, without limitation, the ATUPLEX™ system, the DACC system, the DBTC system and other siRNAlipoplex technology from Silence Therapeutics (London, United Kingdom), STEMFECFM from STEMGENT® (Cambridge, Mass.), and polyethylenimine (PE1) or protamine-based targeted and non-targeted delivery of nucleic acids (Aleku et al. Cancer Res. 2008 68:9788-9798; Strumberg et al. Int J Clin Pharmacol Ther 2012 50:76-78; Santel et al., Gene Ther 2006 13:1222-1234; Santel et al., Gene Ther 200613:1360-1370; Gutbier et al., PulmPharmacol. Ther. 201023:334-344; Kaufmann et al. Microvasc Res 2010 80:286-293; Weide et al. J Immunother. 2009 32:498-507; Weide et al. J Immnnother. 2008 31:180-188; Pascolo Expert Opin. Biol. Ther. 4:1285-1294; Fotin-Mleczek et al., 2011 J. Immunother. 34: 1-15; Song et al., Nature Biotechnol. 2005, 23:709-717; Peer et al., Proc Natl Acad Sci USA. 2007 6; 104:4095-4100; deFougerolles Hum Gene Ther. 2008 19: 125-132; all of which are incorporated herein by reference in their entirety).

In one embodiment such formulations may also be constructed or compositions altered such that they passively or actively are directed to different cell types in vivo, including but not limited to hepatocytes, immune cells, tumor cells, endothelial cells, antigen presenting cells, and leukocytes (Akinc et al. Mol Ther. 2010 18:1357-1364; Song et al., Nat Biotechnol. 2005 23:709-717; Judge et al., J Clin Invest. 2009 119:661-673; Kaufmann et al., Microvasc Res 2010 80:286-293; Santel et al., Gene Ther 200613:1222-1234; Santel et al., Gene Ther 2006 13: 1360-1370; Gutbier et al., Pulm Pharmacol. Ther. 2010 23:334-344; Basha et al., Mol. Ther. 2011 19:2186-2200; Fenske and Cullis, Expert Opin Drug Deliv. 20085:25-44; Peer et al., Science. 2008 319:627-630; Peer and Lieberman, Gene Ther. 2011 18: 1127-1133; all of which are incorporated herein by reference in its entirety). One example of passive targeting of formulations to liver cells includes the DLin-DMA, DLin-KC2-DMA and DLin-MC3-DMA-based lipid nanoparticle formulations which have been shown to bind to apolipoprotein E and promote binding and uptake of these formulations into hepatocytes in vivo (Akinc et al. Mol Ther. 2010 18: 1357-1364; the contents of which are herein incorporated by reference in its entirety). Formulations can also be selectively targeted through expression of different ligands on their surface as exemplified by, but not limited by, folate, transferrin, N-acetylgalactosamine (GaINAc), and antibody targeted approaches (Kolhatkar et al., Curr Drug Discov Technol. 2011 8: 197-206; Musacchio and Torchilin, Front Biosci. 201116: 1388-1412; Yu et al., Mol Membr Biol. 2010 27:286-298; Patil et al., Crit Rev Ther Drug Carrier Syst. 2008 25: 1-61; Benoit et al., Biomacromolecules. 2011 12:2708-2714; Zhao et al., Expert Opin Drug Deliv. 2008 5:309-319; Akinc et al., Mol Ther. 2010 18:1357-1364; Srinivasan et al., Methods Mol Biol. 2012 820: 105-116; Ben-Arie et al., Methods Mol Biol. 2012 757:497-507; Peer 2010 J Control Release. 20:63-68; Peer et al., Proc Natl Acad Sci USA. 2007 104:4095-4100; Kim et al., Methods Mol Biol. 2011 721:339-353; Subramanya et al., Mol Ther. 2010 18:2028-2037; Song et al., Nat Biotechnol. 2005 23:709-717; Peer et al., Science. 2008 319:627-630; Peer and Lieberman, Gene Ther. 2011 18:1127-1133; the contents of all of which are incorporated herein by reference in its entirety).

In one embodiment, a disrupting agent comprising a site-specific FOXP3 targeting moiety of the invention, may be formulated as a solid lipid nanoparticle. A solid lipid nanoparticle (SLN) may be spherical with an average diameter between 10 to 1000 nm. SLN possess a solid lipid core matrix that can solubilize lipophilic molecules and may be stabilized with surfactants and/or emulsifiers. In a further embodiment, the lipid nanoparticle may be a self-assembly lipid-polymer nanoparticle (see Zhang et al., ACS Nano, 2008, 2 (8), pp 1696-1702; herein incorporated by reference in its entirety). As a non-limiting example, the SLN may be the SLN described in PCT Publication No. WO2013/105101, the contents of which are herein incorporated by reference in their entirety. As another non-limiting example, the SLN may be made by the methods or processes described in PCT Publication No. WO 2013/105101, the contents of which are herein incorporated by reference in their entirety.

Liposomes, lipoplexes, or lipid nanoparticles may be used to improve the efficacy of a disrupting agent comprising a site-specific FOXP3 targeting moiety comprising, e.g., a nucleic acid molecule, to direct protein production as these formulations may be able to increase cell transfection by a nucleic acid molecule; and/or increase the translation of encoded protein (e.g., an effector of the invention). One such example involves the use of lipid encapsulation to enable the effective systemic delivery of polyplex plasmid DNA (Heyes et al., Mol Ther. 2007 15:713-720; the contents of which are herein incorporated by reference in its entirety). The liposomes, lipoplexes, or lipid nanoparticles of the invention may also increase the stability of a disrupting agent comprising a site-specific FOXP3 targeting moiety comprising, e.g., a nucleic acid molecule. Liposomes, lipoplexes, or lipid nanoparticles are described in U.S. Patent Publication No. 2016/0038612, the contents of which are incorporated herein by reference in their entirety.

In one embodiment, a disrupting agent comprising a site-specific FOXP3 targeting moiety comprising may be formulated for controlled release and/or targeted delivery. As used herein, “controlled release” refers to a pharmaceutical composition or compound release profile that conforms to a particular pattern of release to effect a therapeutic outcome. In one embodiment, a disrupting agent comprising a site-specific FOXP3 targeting moiety, as described herein, may be encapsulated into a delivery agent described herein and/or known in the art for controlled release and/or targeted delivery. As used herein, the term “encapsulate” means to enclose, surround or encase. As it relates to the formulation of the compounds of the invention, encapsulation may be substantial, complete or partial. The term “substantially encapsulated” means that at least greater than 50, 60, 70, 80, 85, 90, 95, 96, 97, 98, 99, 99.9, 99.9 or greater than 99.999% of the pharmaceutical composition or disrupting agent of the invention may be enclosed, surrounded or encased within the delivery agent. “Partial encapsulation” or “partially encapsulated” means that less than 10, 10, 20, 30, 40 50 or less of the pharmaceutical composition or disrupting agent of the invention may be enclosed, surrounded or encased within the delivery agent. Advantageously, encapsulation may be determined by measuring the escape or the activity of the pharmaceutical composition or compound of the invention using fluorescence and/or electron micrograph. For example, at least 1, 5, 10, 20, 30, 40, 50, 60, 70, 80, 85, 90, 95, 96, 97, 98, 99, 99.9, 99.99 or greater than 99.99% of the pharmaceutical composition or disrupting agent of the invention are encapsulated in the delivery agent.

In one embodiment, a disrupting agent comprising a site-specific FOXP3 targeting moiety comprising as described herein, may be encapsulated in a therapeutic nanoparticle. Therapeutic nanoparticles may be formulated by methods described herein and known in the art such as, but not limited to, PCT Publication Nos. WO 2010/005740, WO 2010/030763, WO 2010/005721, WO 2010/005723, WO 2012/054923, U.S. Patent Publication Nos. 2201/10262491, 2010/0104645, 2010/0087337, 2010/0068285, 2011/0274759, 2010/0068286, 2012/0288541, 2013/0123351, 2013/0230567, 2013/0236500, 2013/0302433, 2013/0302432, 1013/0280339 and 2013/0251757, and U.S. Pat. Nos. 8,206,747, 8,293,276 8,318,208, 8,318,211, 8,623,417, 8,617,608, 8,613,954, 8,613,951, 8,609,142, 8,603,534 and 8,563,041; the contents of each of which is herein incorporated by reference in their entirety. In another embodiment, therapeutic polymer nanoparticles may be prepared by the methods described in U.S. Patent Publication No. 2012/0140790, herein incorporated by reference in its entirety. As a non-limiting example, the therapeutic nanoparticle may comprise about 4 to about 25 weight percent of a disrupting agent and about 10 to about 99 weight percent of a diblock poly (lactic) acid-poly (ethylene)glycol copolymer comprising poly(lactic) acid as described in US Patent Publication No. 2013/0236500, the contents of which are herein incorporated by reference in its entirety. As another non-limiting example, the nanoparticle may comprise about 0.2 to about 35 weight percent of a disrupting agent and about 10 to about 99 weight percent of a diblock poly(lactic) acid-poly(ethylene)glycol copolymer as described in U.S. Patent Publication Nos. 2013/0280339 and 2010251757 and U.S. Pat. No. 8,652,528, the contents of each of which are herein incorporated by reference in their entirety.

In one embodiment, a disrupting agent formulated in therapeutic nanoparticles may be administered intramuscularly, intradermally, or intravenously.

In one embodiment, a disrupting agent may be delivered in therapeutic nanoparticles having a high glass transition temperature such as, but not limited to, the nanoparticles described in US Patent Publication Nos. 2014/0030351 and 2011/0294717, the entire contents of each of which are incorporated herein by reference.

In one embodiment, the therapeutic nanoparticle may be formulated for sustained release. As used herein, “sustained release” refers to a pharmaceutical composition or compound that conforms to a release rate over a specific period of time. The period of time may include, but is not limited to, hours, days, weeks, months and years. As a nonlimiting example, the sustained release nanoparticle may comprise a polymer and a disrupting agent of the present invention (see PCT Publication No. WO2010075072 and U.S. Patent Publication Nos. 2010/0216804, 2011/0217377, 2012/0201859, 2013/0243848 and 2013/0243827, each of which is herein incorporated by reference in their entirety).

In one embodiment, a disrupting agent of the invention may be encapsulated in, linked to and/or associated with synthetic nanocarriers. Synthetic nanocarriers include, but are not limited to, those described in PCT Publication. Nos. WO 2010/005740, WO 2010/030763, WO 2012/13501, WO 2012/149252, WO 2012149255, WO 2012149259, WO 2012149265, WO 2012149268, WO 2012149282, WO 2012149301, WO 2012149393, WO 2012149405, WO 2012149411 and WO 2012149454 and US Patent Publication Nos. 20110262491, 20100104645, 20100087337, 20120244222 and 0520130236533, and U.S. Pat. No. 8,652,487, the contents of each of which is herein incorporated by reference in their entirety. The synthetic nanocarriers may be formulated using methods known in the art and/or described herein. As a nonlimiting example, the synthetic nanocarriers may be formulated by the methods described in PCT Publication Nos. WO 2010005740, WO 2010030763 and WO 201213501 and US Patent Publication Nos. 20110262491, 20100104645, 20100087337 and 20120244222, each of which is herein incorporated by reference in their entirety. In another embodiment, the synthetic nanocarrier formulations may be lyophilized by methods described in PCT Publication No. WO 2011072218 and U.S. Pat. No. 8,211,473; each of which is herein incorporated by reference in their entirety. In yet another embodiment, formulations of the present invention, including, but not limited to, synthetic nanocarriers, may be lyophilized or reconstituted by the methods described in US Patent Publication No. 20130230568, the contents of which are herein incorporated by reference in its entirety.

In one embodiment, synthetic nanocarriers comprising a disrupting agent may be administered intramuscularly, intradermally, or intravenously.

In some embodiments, a disrupting agent may be formulated for delivery using smaller LNPs. Such particles may comprise a diameter from below 0.1 μm up to 1000 μm such as, but not limited to, less than 0.1 μm, less than 1.0 μm, less than 5 μm, less than 10 μm, less than 15 μm, less than 20 μm, less than 25 μm, less than 30 μm, less than 35 μm, less than 40 μm, less than 50 μm, less than 55 μm, less than 60 μm, less than 65 μm, less than 70 μm, less than 75 μm, less than 80 μm, less than 85 μm, less than 90 μm, less than 95 μm, less than 100 μm, less than 125 μm, less than 150 μm, less than 175 μm, less than 200 μm, less than 225 μm, less than 250 μm, less than 275 μm, less than 300 μm, less than 325 μm, less than 350 μm, less than 375 μm, less than 400 μm, less than 425 μm, less than 450 μm, less than 475 μm, less than 500 μm, less than 525 μm, less than 550 μm, less than 575 μm, less than 600 μm, less than 625 μm, less than 650 μm, less than 675 μm, less than 700 μm, less than 725 μm, less than 750 μm, less than 775 μm, less than 800 μm, less than 825 μm, less than 850 μm, less than 875 μm, less than 900 μm, less than 925 μm, less than 950 μm, less than 975 μm.

In another embodiment, a disrupting agent may be formulated for delivery using smaller LNPs which may comprise a diameter from about 1 nm to about 100 nm, from about 1 nm to about 10 nm, about 1 nm to about 20 nm, from about 1 nm to about 30 nm, from about 1 nm to about 40 nm, from about 1 nm to about 50 nm, from about 1 nm to about 60 nm, from about 1 nm to about 70 nm, from about 1 nm to about 80 nm, from about 1 nm to about 90 nm, from about 5 nm to about from 100 nm, from about 5 nm to about 10 nm, about 5 nm to about 20 nm, from about 5 nm to about 30 nm, from about 5 nm to about 40 nm, from about 5 nm to about 50 nm, from about 5 nm to about 60 nm, from about 5 nm to about 70 nm, from about 5 nm to about 80 nm, from about 5 nm to about 90 nm, about 10 to about 50 nm, from about 20 to about 50 nm, from about 30 to about 50 nm, from about 40 to about 50 nm, from about 20 to about 60 nm, from about 30 to about 60 nm, from about 40 to about 60 nm, from about 20 to about 70 nm, from about 30 to about 70 nm, from about 40 to about 70 nm, from about 50 to about 70 nm, from about 60 to about 70 nm, from about 20 to about 80 nm, from about 30 to about 80 nm, from about 40 to about 80 nm, from about 50 to about 80 nm, from about 60 to about 80 nm, from about 20 to about 90 nm, from about 30 to about 90 nm, from about 40 to about 90 nm, from about 50 to about 90 nm, from about 60 to about 90 nm and/or from about 70 to about 90 nm.

In one embodiment, a disrupting agent may be formulated in smaller LNPs and may be administered intramuscularly, intrademrally, or intravenously.

In one embodiment, a disrupting agent may be formulated for delivery using the drug encapsulating microspheres described in PCT Patent Publication No. WO 2013063468 or U.S. Pat. No. 8,440,614, each of which is herein incorporated by reference in its entirety. In another aspect, the amino acid, peptide, polypeptide, lipids (APPL) are useful in delivering the disrupting agents of the invention to cells (see PCT Patent Publication No. WO 2013063468, herein incorporated by reference in its entirety).

In one aspect, the lipid nanoparticle may be a limit size lipid nanoparticle described in PCT Patent Publication No. WO 2013059922, herein incorporated by reference in its entirety. The limit size lipid nanoparticle may comprise a lipid bilayer surrounding an aqueous core or a hydrophobic core; where the lipid bilayer may comprise a phospholipid such as, but not limited to, diacylphosphatidylcholine, a diacylphosphatidylethanolamine, a ceramide, a sphingomyelin, a dihydrosphingomyelin, a cephalin, a cerebroside, a C8-C20 fatty acid diacylphophatidylcholine, and I-palmitoyl-2-oleoyl phosphatidylcholine (POPC). In another aspect the limit size lipid nanoparticle may comprise a polyethylene glycol-lipid such as, but not limited to, DLPEPEG, DMPE-PEG, DPPC-PEG and DSPE-PEG.

In one embodiment, a disrupting agent of the invention may be delivered, localized and/or concentrated in a specific location using the delivery methods described in PCT Patent Publication No. WO 2013063530, the contents of which are herein incorporated by reference in its entirety. As a non-limiting example, a subject may be administered an empty polymeric particle prior to, simultaneously with or after delivering the disrupting agent to the subject. The empty polymeric particle undergoes a change in volume once in contact with the subject and becomes lodged, embedded, immobilized or entrapped at a specific location in the subject.

In one embodiment, a disrupting agent may be formulated in an active substance release system (See e.g., US Patent Publication No. 20130102545, herein incorporated by reference in its entirety). The active substance release system may comprise 1) at least one nanoparticle bonded to an oligonucleotide inhibitor strand which is hybridized with a catalytically active nucleic acid and 2) a compound bonded to at least one substrate molecule bonded to a therapeutically active substance (e.g., a disrupting agent of the invention), where the therapeutically active substance is released by the cleavage of the substrate molecule by the catalytically active nucleic acid.

In one embodiment, the nanoparticles of the present invention may be water soluble nanoparticles such as, but not limited to, those described in PCT Publication No. WO 2013090601, the contents of which are herein incorporated by reference in its entirety. The nanoparticles may be inorganic nanoparticles which have a compact and zwitterionic ligand in order to exhibit good water solubility. The nanoparticles may also have small hydrodynamic diameters (HD), stability with respect to time, pH, and salinity and a low level of non-specific protein binding.

In one embodiment, the nanoparticles of the present invention are stealth nanoparticles or target-specific stealth nanoparticles such as, but not limited to, those described in U.S. Patent Publication Nos. 20130172406 (Bind), US20130251817 (Bind), 2013251816 (Bind) and 20130251766 (Bind), the contents of each of which are herein incorporated by reference in its entirety. The stealth nanoparticles may comprise a diblock copolymer and a chemotherapeutic agent. These stealth nanoparticles may be made by the methods described in us Patent Publication Nos. 20130172406, 20130251817, 2013251816 and 20130251766, the contents of each of which are herein incorporated by reference in its entirety. As a non-limiting example, the stealth nanoparticles may target cancer cells such as the nanoparticles described in US Patent Publication Nos. 20130172406, 20130251817, 2013251816 and 20130251766, the contents of each of which are herein incorporated by reference in its entirety.

In one embodiment, stealth nanoparticles comprising a disrupting agent of the invention may be administered intramuscularly, intradermally, or intravenously.

In one embodiment, a disrupting agent of the invention may be formulated in and/or delivered in a lipid nanoparticle comprising a plurality of cationic lipids such as, but not limited to, the lipid nanoparticles described in US Patent Publication No. 20130017223, the contents of which are herein incorporated by reference in its entirety. As a non-limiting example, the LNP formulation may comprise a first cationic lipid and a second cationic lipid. As another non-limiting example, the LNP formulation may comprise DLin-MC2-DMA and DLinMC4-DMA. As yet another non-limiting example, the LNP formulation may comprise DLin-MC3-DMA and CI2-200. In one embodiment, the LNP formulations comprising a plurality of cationic lipids (such as, but not limited to, those described in US Patent Publication No. US20130017223, the contents of which are herein incorporated by reference in its entirety) and may be administered intramuscularly, intradermally, or intravenously.

In one embodiment, a disrupting agent as described herein, may be formulated in and/or delivered in a lipid nanoparticle comprising the cationic lipid DLin-MC3-DMA and the neutral lipid DOPE. The lipid nanoparticle may also comprise a PEG based lipid and a cholesterol or antioxidant. These lipid nanoparticle formulations comprising DLin-MC3-DMA and DOPE and a disrupting agent may be administered intramuscularly, intradermally, or intravenously.

In one embodiment, the lipid nanoparticle comprising DLin-MC3-DMA and DOPE may comprise a PEG lipid such as, but not limited to, pentaerythritol PEG ester tetrasuccinimidyl and pentaerythritol PEG ether tetra-thiol, PEGc-DOMG, PEG-DMG (1,2-Dimyristoyl-sn-glycerol, methoxypolyethylene Glycol), PEG-DSG (1,2-Distearoyl-snglycerol, methoxypolyethylene Glycol), PEG-DPG (1,2-Dipalmitoyl-sn-glycerol, methoxypolyethylene glycol), PEG-DSA (PEG coupled to 1,2-distearyloxypropyl-3-amine), PEG-DMA (PEG coupled to 1,2-dimyristyloxypropyl-3-amine, PEG-c-DNA, PEG-c-DMA, PEG-S-DSG, PEG-c-DMA, PEG-DPG, PEG-DMG 2000 and those described herein and/or known in the art.

In one embodiment, the lipid nanoparticle comprising DLin-MC3-DMA and DOPE may include 0.5% to about 3.0%, from about 1.0% to about 3.5%, from about 1.5% to about 4.0%, from about 2.0% to about 4.5%, from about 2.5% to about 5.0% and/or from about 3.0% to about 6.0% of the lipid molar ratio of a PEG lipid.

In one embodiment, the lipid nanoparticle comprising DLin-MC3-DMA and DOPE may include 25.0% cholesterol to about 50.0% cholesterol, from about 30.0% cholesterol to about 45.0% cholesterol, from about 35.0% cholesterol to about 50.0% cholesterol and/or from about 48.5% cholesterol to about 60% cholesterol. In one embodiment, formulations may comprise a percentage of cholesterol selected from the group consisting of 28.5%, 31.5%, 33.5%, 36.5%, 37.0%, 38.5%, 39.0%, 43.5% and 48.5%.

In one embodiment, the lipid nanoparticle comprising DLin-MC3-DMA and DOPE may include 25.0% antioxidant to about 50.0% antioxidant, from about 30.0% antioxidant to about 45.0% antioxidant, from about 35.0% antioxidant to about 50.0% antioxidant and/or from about 48.5% antioxidant to about 60% antioxidant. In one embodiment, formulations may comprise a percentage of antioxidant selected from the group consisting of 28.5%, 31.5%, 33.5%, 36.5%, 37.0%, 38.5%, 39.0%, 43.5% and 48.5%.

The disrupting agent of the invention can be formulated using natural and/or synthetic polymers. Non-limiting examples of polymers which may be used for delivery include, but are not limited to, DYNAMIC POLYCONJUGATE® (Arrowhead Research Corp., Pasadena, Calif.) formulations from MIRUS® Bio (Madison, Wis.) and Roche Madison (Madison, Wis.), PHASERX™ polymer formulations such as, without limitation, SMARTT POLYMER TECHNOLOGY™ (Seattle, Wash.), DMRIIDOPE, poloxamer, VAXFECTIN® adjuvant from Vical (San Diego, Calif.), chitosan, cyclodextrin from Calando Pharmaceuticals (Pasadena, Calif.), dendrimers and poly(lactic-co-glycolic acid) (PLGA) polymers, RONDEL™ (RNAi/Oligonucleotide Nanoparticle Delivery) polymers (Arrowhead Research Corporation, Pasadena, Calif.) and pH responsive co-block polymers such as, but not limited to, PHASERX™ (Seattle, Wash.).

The polymer formulations may permit the sustained or delayed release of a disrupting agent (e.g., following intramuscular, intradermal or subcutaneous injection). The altered release profile of the disrupting agent can result in, for example, translation of an encoded protein over an extended period of time. The polymer formulation may also be used to increase the stability of the disrupting agent. For example, biodegradable polymers have been previously used to protect nucleic acids other than modified mRNA from degradation and been shown to result in sustained release of payloads in vivo (Rozema et al., Proc Natl Acad Sci USA. 2007 104:12982-12887; Sullivan et al., Expert Opin Drug Deliv. 2010 7:1433-1446; Convertine et al., Biomacromolecules. 2010 Oct. 1; Chu et al., Acc Chern Res. 2012 Jan. 13; Manganiello et al., Biomaterials. 2012 33:2301-2309; Benoit et al., Biomacromolecules. 2011 12:2708-2714; Singha et al., Nucleic Acid Ther. 2011 2: 133-147; deFougerolles Hum Gene Ther. 2008 19:125-132; Schaffert and Wagner, Gene Ther. 2008 16:1131-1138; Chaturvedi et al., Expert Opin Drug Deliv. 2011 8: 1455-1468; Davis, Mol Pharm. 2009 6:659-668; Davis, Nature 201 0464: 1067-1070; each of which is herein incorporated by reference in its entirety).

In one embodiment, the pharmaceutical compositions may be sustained release formulations. In a further embodiment, the sustained release formulations may be for subcutaneous delivery. Sustained release formulations may include, but are not limited to, PLGA microspheres, ethylene vinyl acetate (EVAc), poloxamer, GELSITE® (Nanotherapeutics, Inc. Alachua, Fla.), HYLENEX® (Halozyme Therapeutics, San Diego Calif.), surgical sealants such as fibrinogen polymers (Ethic on Inc. Cornelia, Ga.), TISSELL® (Baxter International, Inc Deerfield, Ill.), PEG-based sealants, and COSEAL® (Baxter International, Inc Deerfield, Ill.).

B. Vector Encoded Site-Specific FOXP3 Disrupting Agents of the Invention

Disrupting agents comprising a site-specific FOXP3 targeting moiety, e.g., comprising a nucleic acid molecule, may be expressed from transcription units inserted into DNA or RNA vectors (see, e.g., Couture, A, et al., TIG. (1996), 12:5-10; WO 00/22113, WO 00/22114, and U.S. Pat. No. 6,054,299). In some embodiment, expression is sustained (months or longer), depending upon the specific construct used and the target tissue or cell type. These transgenes can be introduced as a linear construct, a circular plasmid, or a viral vector, which can be an integrating or non-integrating vector. The transgene can also be constructed to permit it to be inherited as an extrachromosomal plasmid (Gassmann, et al., (1995) Proc. Natl. Acad. Sci. USA 92:1292). Different components of the disrupting agent, e.g., gRNA and effector, can be located on separate expression vectors that can be co-introduced (e.g., by transfection or infection) into a target cell. Alternatively, each individual component can be transcribed by promoters both of which are located on the same expression plasmid.

Delivery of a disrupting agent expressing vector can be systemic, such as by intravenous or intramuscular administration, by administration to target cells ex-planted from the patient followed by reintroduction into the patient, or by any other means that allows for introduction into a desired target cell.

In certain embodiment, the nucleic acids described herein or the nucleic acids encoding a protein described herein, e.g., an effector, are incorporated into a vector, e.g., a viral vector.

The individual strand or strands of a disrupting agent comprising a site-specific FOXP3 targeting moiety comprising a nucleic acid molecule can be transcribed from a promoter in an expression vector. Where two separate strands are to be expressed to generate, for example, a dsRNA, two separate expression vectors can be co-introduced (e.g., by transfection or infection) into a target cell. Alternatively, each individual strand of a nucleic acid molecule can be transcribed by promoters both of which are located on the same expression plasmid. In one embodiment, a nucleic acid molecule is expressed as inverted repeat polynucleotides joined by a linker polynucleotide sequence such that the nucleic acid molecule has a stem and loop structure.

Expression vectors are generally DNA plasmids or viral vectors. Expression vectors compatible with eukaryotic cells, preferably those compatible with vertebrate cells, can be used to produce recombinant constructs for the expression of a disrupting agent as described herein.

Constructs for the recombinant expression of a disrupting agent will generally require regulatory elements, e.g., promoters, enhancers, etc., to ensure the expression of the disrupting agent in target cells.

Expression of natural or synthetic nucleic acids is typically achieved by operably linking a nucleic acid encoding the nucleic acid of interest to a regulatory region, such as a promoter, and incorporating the construct into an expression vector. The vectors can be suitable for replication and integration in eukaryotes.

Regulatory regions, such as a promoter, suitable for operable linking to a nucleic acid molecules can be operably linked to a regulatory region such as a promoter. can be from any species. Any type of promoter can be operably linked to a nucleic acid sequence. Examples of promoters include, without limitation, tissue-specific promoters, constitutive promoters, and promoters responsive or unresponsive to a particular stimulus (e.g., inducible promoters). Additional promoter elements, e.g., enhancing sequences, regulate the frequency of transcriptional initiation. Typically, these are located in the region 30-110 bp upstream of the start site, although a number of promoters have recently been shown to contain functional elements downstream of the start site as well. The spacing between promoter elements frequently is flexible, so that promoter function is preserved when elements are inverted or moved relative to one another. In the thymidine kinase (tk) promoter, the spacing between promoter elements can be increased to 50 bp apart before activity begins to decline. Depending on the promoter, individual elements can function either cooperatively or independently to activate transcription.

One example of a suitable promoter is the immediate early cytomegalovirus (CMV) promoter sequence. This promoter sequence is a strong constitutive promoter sequence capable of driving high levels of expression of any polynucleotide sequence operatively linked thereto. Another example of a suitable promoter is Elongation Growth Factor-1a (EF-1a). However, other constitutive promoter sequences may also be used, including, but not limited to the simian virus 40 (SV40) early promoter, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, a Rous sarcoma virus promoter, as well as human gene promoters such as, but not limited to, the actin promoter, the myosin promoter, the hemoglobin promoter, and the creatine kinase promoter.

Further, the present invention should not be limited to the use of constitutive promoters. Inducible promoters are also contemplated as part of the invention. The use of an inducible promoter provides a molecular switch capable of turning on expression of the polynucleotide sequence which it is operatively linked when such expression is desired, or turning off the expression when expression is not desired. Examples of inducible promoters include, but are not limited to a metallothionine promoter, a glucocorticoid promoter, a progesterone promoter, and a tetracycline promoter.

Additional regulatory regions that may be useful in nucleic acid constructs, include, but are not limited to, transcription and translation terminators, initiation sequences, polyadenylation sequences, translation control sequences (e.g., an internal ribosome entry segment, IRES), enhancers, inducible elements, or introns. Such regulatory regions may not be necessary, although they may increase expression by affecting transcription, stability of the mRNA, translational efficiency, or the like. Such regulatory regions can be included in a nucleic acid construct as desired to obtain optimal expression of the nucleic acids in the cell(s). Sufficient expression, however, can sometimes be obtained without such additional elements.

The expression vector to be introduced can also contain either a selectable marker gene or a reporter gene or both to facilitate identification and selection of expressing cells from the population of cells sought to be transfected or infected through viral vectors. In other aspects, the selectable marker may be carried on a separate piece of DNA and used in a co-transfection procedure. Both selectable markers and reporter genes may be flanked with appropriate transcriptional control sequences to enable expression in the host cells. Useful selectable markers include, for example, antibiotic-resistance genes, such as neo and the like. Non-limiting examples of selectable markers include puromycin, ganciclovir, adenosine deaminase (ADA), aminoglycoside phosphotransferase (neo, G418, APH), dihydrofolate reductase (DHFR), hygromycin-B-phosphtransferase, thymidine kinase (TK), and xanthin-guanine phosphoribosyltransferase (XGPRT). Such markers are useful for selecting stable transformants in culture. Other selectable markers include fluorescent polypeptides, such as green fluorescent protein or yellow fluorescent protein.

Signal peptides may also be included and can be used such that an encoded polypeptide is directed to a particular cellular location (e.g., the cell surface).

Reporter genes may be used for identifying potentially transfected cells and for evaluating the functionality of transcriptional control sequences. In general, a reporter gene is a gene that is not present in or expressed by the recipient source and that encodes a polypeptide whose expression is manifested by some easily detectable property, e.g., enzymatic activity. Expression of the reporter gene is assayed at a suitable time after the DNA has been introduced into the recipient cells. Suitable reporter genes may include genes encoding luciferase, beta-galactosidase, chloramphenicol acetyl transferase, secreted alkaline phosphatase, or the green fluorescent protein gene (e.g., Ui-Tei et al., 2000 FEBS Letters 479: 79-82). Suitable expression systems are well known and may be prepared using known techniques or obtained commercially. In general, the construct with the minimal 5′ flanking region showing the highest level of expression of reporter gene is identified as the promoter. Such promoter regions may be linked to a reporter gene and used to evaluate agents for the ability to modulate promoter-driven transcription.

Other aspects to consider for vectors and constructs are known in the art.

In some embodiments, a vector, e.g., a viral vector comprises a disrupting agent comprising a site-specific FOXP3 targeting moiety comprising a nucleic acid molecule.

Viral vector systems which can be utilized with the methods and compositions described herein include, but are not limited to, (a) adenovirus vectors (e.g., an Ad5/F35 vector); (b) retrovirus vectors, including but not limited to lentiviral vectors (including integration competent or integration-defective lentiviral vectors), moloney murine leukemia virus, etc.; (c) adeno-associated virus vectors; (d) herpes simplex virus vectors; (e) SV 40 vectors; (f) polyoma virus vectors; (g) papilloma virus vectors; (h) picornavirus vectors; (i) pox virus vectors such as an orthopox, e.g., vaccinia virus vectors or avipox, e.g. canary pox or fowl pox; and (j) a helper-dependent or gutless adenovirus. Replication-defective viruses can also be advantageous. Different vectors will or will not become incorporated into the cells' genome. The constructs can include viral sequences for transfection, if desired. Alternatively, the construct can be incorporated into vectors capable of episomal replication, e.g. EPV and EBV vectors. See, e.g., U.S. Pat. Nos. 6,534,261; 6,607,882; 6,824,978; 6,933,113; 6,979,539; 7,013,219; and 7,163,824, the entire contents of each of which is incorporated by reference herein.

Vectors, including those derived from retroviruses such as lentivinis, are suitable tools to achieve long-term gene transfer since they allow long-term, stable integration of a transgene and its propagation in daughter cells. Examples of vectors include expression vectors, replication vectors, probe generation vectors, and sequencing vectors. The expression vector may be provided to a cell in the form of a viral vector. Viral vector technology is well known in the art, and described in a variety of virology and molecular biology manuals.

In one embodiment, a suitable viral vector for use in the present invention is an adeno-associated viral vector, such as a recombinant adeno-associate viral vector.

Recombinant adeno-associated virus vectors (rAAV) are gene delivery systems based on the defective and nonpathogenic parvovirus adeno-associated type 2 virus. All vectors are derived from a plasmid that retains only the AAV 145 bp inverted terminal repeats flanking the transgene expression cassette. Efficient gene transfer and stable transgene delivery due to integration into the genomes of the transduced cell are key features for this vector system. (Wagner et al., Lancet 351:9117 1702-3 (1998), Kearns et al., Gene Ther. 9:748-55 (1996)). AAV serotypes, including AAV1, AAV2, AAV3, AAV4, AAV5, AAV6, AAV7, AAV8 and AAV9, can be used in accordance with the present invention.

Replication-deficient recombinant adenoviral vectors (Ad) can be produced at high titer and readily infect a number of different cell types. Most adenovirus vectors are engineered such that a transgene replaces the Ad E1a, E1b, and/or E3 genes; subsequently the replication defective vector is propagated in human 293 cells that supply deleted gene function in trans. Ad vectors can transduce multiple types of tissues in vivo, including nondividing, differentiated cells such as those found in liver, kidney and muscle. Conventional Ad vectors have a large carrying capacity. An example of the use of an Ad vector in a clinical trial involved polynucleotide therapy for antitumor immunization with intramuscular injection (Sterman et al., Hum. Gene Ther. 7:1083-9 (1998)). Additional examples of the use of adenovirus vectors for gene transfer in clinical trials include Rosenecker et al., Infection 24:1 5-10 (1996); Sterman et al., Hum. Gene Ther. 9:7 1083-1089 (1998); Welsh et al., Hum. Gene Ther. 2:205-18 (1995); Alvarez et al., Hum. Gene Ther. 5:597-613 (1997); Topf et al., Gene Ther. 5:507-513 (1998); Sterman et al., Hum. Gene Ther. 7:1083-1089 (1998).

Packaging cells are used to form virus particles that are capable of infecting a host cell. Such cells include 293 cells, which package adenovirus, and ψ2 cells or PA317 cells, which package retrovirus. Viral vectors used in gene therapy are usually generated by a producer cell line that packages a nucleic acid vector into a viral particle. The vectors typically contain the minimal viral sequences required for packaging and subsequent integration into a host (if applicable), other viral sequences being replaced by an expression cassette encoding the protein to be expressed. The missing viral functions are supplied in trans by the packaging cell line. For example, AAV vectors used in gene therapy typically only possess inverted terminal repeat (ITR) sequences from the AAV genome which are required for packaging and integration into the host genome. Viral DNA is packaged in a cell line, which contains a helper plasmid encoding the other AAV genes, namely rep and cap, but lacking ITR sequences. The cell line is also infected with adenovirus as a helper. The helper virus promotes replication of the AAV vector and expression of AAV genes from the helper plasmid. The helper plasmid is not packaged in significant amounts due to a lack of ITR sequences. Contamination with adenovirus can be reduced by, e.g., heat treatment to which adenovirus is more sensitive than AAV.

### IV. Methods of the Invention

A. Modulation of Expression of FOXP3 in a Cell

The present invention also provides methods of use of the agents and compositions described herein to modulate expression of forkhead box P3 (FOXP3) in a cell. The methods include contacting the cell, e.g., a naïve T cell, with a site-specific FOXP3 disrupting agent, the disrupting agent comprising a site-specific FOXP3 targeting moiety which targets a FOXP3 expression control region, and an effector molecule, thereby modulating expression of FOXP3 in the cell. The site-specific disrupting agent, the effector, or both the site-specific disrupting agent and the effector may be present in a composition, such as a composition described above. In some embodiments, the site-specific disrupting agent and the effector are present in the same compositions. In other embodiments, the site-specific disrupting agent and the effector are present in different compositions. In some embodiments, the methods of the invention include contacting a cell with two site-specific FOXP3 disrupting agents (a first and a second agent). The two site specific FOXP3 disrupting agents may be present in the same composition, e.g., pharmaceutical composition, e.g., pharmaceutical composition comprising an LNP, or in separate compositions, e.g., pharmaceutical composition, e.g., pharmaceutical composition comprising an LNP. The cell may be contacted with the first site specific FOXP3 disrupting agent at one time and contacted with the second site specific FOXP3 disrupting agent at a second time, or the cell may be contacted with both agents at the same time.

Expression of FOXP3 may be enhanced or reduced as compared to, for example, a cell that was not contacted with the site-specific FOXP3 disrupting agent. Modulation in gene expression can be assessed by any methods known in the art. For example, a modulation in the expression may be determined by determining the mRNA expression level of a gene, e.g., in a cell, a plurality of cells, and/or a tissue sample, using methods routine to one of ordinary skill in the art, e.g., northern blotting, qRT-PCR; by determining the protein level of a gene using methods routine to one of ordinary skill in the art, such as western blotting, immunological techniques.

The term “reduced” in the context of the level of FOXP3 gene expression or FOXP3 protein production in a subject, or a disease marker or symptom refers to a statistically significant decrease in such level. The decrease can be, for example, at least 20%, 25%, 30%, 35%, 40%, 45%, 50%, 55%, 60%, 65%, 70%, 75%, 80%, 85%, 90%, or 95%, or below the level of detection for the detection method. In certain embodiments, the expression of the target is normalized, i.e., decreased towards or to a level accepted as within the range of normal for an individual without such disorder. As used here, “lower” in a subject can refer to lowering of gene expression or protein production in a cell in a subject does not require lowering of expression in all cells or tissues of a subject. For example, as used herein, lowering in a subject can include lowering of gene expression or protein production in the liver of a subject.

The term “reduced” can also be used in association with normalizing a symptom of a disease or condition, i.e. decreasing the difference between a level in a subject suffering from an autoimmune disease or a FOXP3-associated disease towards or to a level in a normal subject not suffering from an autoimmune disease or a FOXP3-associated disease. As used herein, if a disease is associated with an elevated value for a symptom, “normal” is considered to be the upper limit of normal. If a disease is associated with a decreased value for a symptom, “normal” is considered to be the lower limit of normal.

The term “enhanced” in the context of the level of FOXP3 gene expression or FOXP3 protein production in a subject, or a disease marker or symptom refers to a statistically significant increase in such level. The increase can be, for example, at least 20%, 25%, 30%, 35%, 40%, 45%, 50%, 55%, 60%, 65%, 70%, 75%, 80%, 85%, 90%, or 95%, or above the level of detection for the detection method. In certain embodiments, the expression of the target is normalized, i.e., increase towards or to a level accepted as within the range of normal for an individual without such disorder. As used here, “higher” in a subject can refer to increasing gene expression or protein production in a cell in a subject does not require increasing expression in all cells or tissues of a subject. For example, as used herein, increasing in a subject can include increasing gene expression or protein production in the liver of a subject.

The term “enhanced” can also be used in association with normalizing a symptom of a disease or condition, i.e. increasing the difference between a level in a subject suffering from a FOXP3-associated disease or an autoimmune disease towards or to a level in a normal subject not suffering from a FOXP3-associated disease or an autoimmune disease. As used herein, if a disease is associated with an elevated value for a symptom, “normal” is considered to be the upper limit of normal. If a disease is associated with a decreased value for a symptom, “normal” is considered to be the lower limit of normal.

In some embodiments, a suitable cell for use in the methods of the invention is a mammalian cell. In some embodiments, the cell is a somatic cell. In some embodiments, the cell is a primary cell. For example, in some embodiments, the cell is a mammalian somatic cell. In some embodiments, the mammalian somatic cell is a primary cell. In some embodiments, the mammalian somatic cell is a non-embryonic cell.

B. In Vitro Generation of Immune Cells

The step of contacting may be performed in vitro, in vivo (i.e., the cell may be within a subject), or ex vivo. In some embodiments, contacting a cell is performed ex vivo and the methods further include, prior to the step of contacting, a step of removing the cell (e.g., a mammalian cell) from a subject. In some embodiments, the methods further comprise, after the step of contacting, a step of (b) administering the cell (e.g., mammalian cells) to a subject.

The present invention provides methods of generating immune cells, e.g., Tregs, which, in one aspect of the invention include a site-specific FOXP3 disrupting agent of the invention. The FOXP3 disrupting agent may modulate, e.g., increase, the expression of FOXP3 gene for a period of time that is sufficient to direct the immune cells to a differentiation pathway or alter the activation status, for example, to induce a naïve T cell to differentiate into a Treg cell or to activate a Treg cell.

Methods for the Manipulation of Immune Cells

In one embodiment, the instant invention provides a method for manipulating cells, e.g., immune cells or a sub-population thereof (e.g., Tregs or naïve T cells). In this context, the term “manipulation” includes, for example, activation, division, differentiation, growth, expansion, reprogramming, anergy, quiescence, senescence, apoptosis or death of the target cells.

A variety of cells, e.g., immune cells, may be manipulated, including, fresh samples derived from subjects, primary cultured cells, immortalized cells, cell-lines, hybridomas, etc. The cells to be manipulated may also include stem cells, such as embryonic stem cells, induced pluripotent stem cells, mobilized peripheral blood stem cells. The manipulated cells may be used for various immunotherapeutic applications as well as for research.

In certain embodiments of the invention, the cells may be manipulated ex vivo by culturing a sample containing immune cells, such as a sample obtained from a subject, such as a subject that would benefit from modulating the expression of FOXP3, with the FOXP3 disrupting agent of the invention.

In certain embodiments, the immune cells to be manipulated may be naïve T cells isolated from cord blood or peripheral blood. The naïve T cells may be manipulated (e.g., differentiated and/or activated) by contacting the cells with a FOXP3 disrupting agent of the invention. In some embodiments, the naïve T cells may further be contacted with an antigen or an antigen presenting cell to differentiate into antigen specific Tregs. In some embodiment, the immune cells to be manipulated may be Treg cells. Tregs may be manipulated (e.g., activated) by contacting the cells with the FOXP3 disrupting agent of the invention.

Methods to isolate the foregoing T cells from a sample, such as a sample derived from a subject, are known in the art and described below.

As used herein, the term “regulatory T cells,” “Treg cells,” or “Tregs,” also known as “suppressor T cells,” refers to a population of T cells which modulate the immune system, maintain tolerance to self-antigens, and prevent autoimmune disease. Tregs are immunosuppressive and generally suppress or downregulate induction and proliferation of effector T cells. Tregs express the biomarkers CD4, FOXP3, and CD25 and are thought to be derived from the same lineage as naïve CD4 cells.

As used herein, the term “naïve T cells,” refers to a population of T cells that has differentiated in bone marrow, and successfully undergone the positive and negative processes of central selection in the thymus. Among these are the naïve forms of helper T cells (CD4+) and cytotoxic T cells (CD8+). A naïve T cell is considered mature and, unlike activated or memory T cells, has not encountered its cognate antigen within the periphery.

Expansion of T Cell Population

In a related embodiment, the present invention further relates to methods for expanding certain immune cells, such as naïve T cells or Tregs from a population of immune cells, e.g., expanding Tregs or naïve T cells contained in sample containing B-cells, dendritic cells, macrophages, plasma cells, and the like. In another embodiment, the present invention also relates to methods for expanding a specific population of T-cells, e.g., expanding differentiated/activated Tregs.

In one embodiment, the immune cells, e.g., Tregs, are expanded (e.g., grown or differentiated) ex vivo by culturing a sample containing immune cells with the FOXP3 disrupting agent of the invention. In one embodiment, ex vivo T cell expansion can be performed by first isolating Tregs or naïve T cells from a sample and subsequently stimulating T-cells by contacting them with the FOXP3 disrupting agent of the invention, such that the Tregs are activated, and/or expanded.

In one embodiment of the invention, the T cells are primary T-cells obtained from a subject. T-cells can be obtained from a number of sources, including peripheral blood mononuclear cells, bone marrow, lymph node tissue, cord blood, thymus tissue, tissue from a site of infection, spleen tissue, and tumors. In certain embodiments of the present invention, any number of primary T-cells and/or T-cell lines available in the art, may be used.

Studies on whole blood counts reveal that the number of T-cells in whole blood is very low. For example, according to the product catalog published by Stem Cell Technologies, Vancouver, BC, CANADA (Document #23629, VERSION 2.1.0), the leukocyte population in whole blood is about 0.1-0.2% (due to predominance of erythrocytes), of which T-cells make up about 7-24% of the overall leukocyte population. Among T-cells, CD4+ T-cells make up about 4-20% of the overall leukocyte population (translating to less than 0.04% of the overall cell population in whole blood) and CD8+ T-cells make up about 2-11% of the overall leukocyte population (translating to less than 0.022% of the overall cell population in whole blood). Thus, in certain embodiments of the present invention, methods of the invention may be coupled with other art-known techniques for enrichment of immune cells, e.g., naïve T cells or Tregs. The enrichment step may be carried out prior to contacting the sample with the FOXP3 disrupting agent of the instant invention. In another embodiment, the enrichment step may be carried out after the sample has been contacted with the FOXP3 disrupting agent of the present invention.

In one embodiment, the Tregs population may be enriched using FICOLL separation. In one embodiment, cells from the circulating blood of an individual are obtained by apheresis or leukapheresis. The apheresis product typically contains lymphocytes, including T cells, monocytes, granulocytes, B cells, other nucleated white blood cells, red blood cells, and platelets. The cells collected by apheresis may be washed to remove the plasma fraction and to place the cells in an appropriate buffer or media for subsequent processing steps. The cells are then washed with phosphate buffered saline (PBS). Alternately, the wash solution lacks calcium and may lack magnesium or may lack many if not all divalent cations. A semi-automated “flow-through” centrifuge may also be used according to the manufacturer's instructions. After washing, the cells may be resuspended in a variety of biocompatible buffers, such as, for example, Ca-free, Mg-free PBS. Alternatively, the undesirable components of the apheresis sample may be removed and the cells directly resuspended in culture media.

In another embodiment, peripheral or whole blood T cells may be enriched by lysing the red blood cells and depleting the monocytes, for example, by centrifugation through a PERCOLL™ gradient. A specific subpopulation of T cells, such as CD28+, CD4+, CD8+, CD45RA+, and CD45RO+T cells, can be further isolated by positive or negative selection techniques.

In accordance with the present invention, various sorting techniques may be optionally employed. For example, the expanded or manipulated T cell population may be further sorted using a combination of antibodies directed to surface markers unique to the cells. A preferred method is cell sorting and/or selection via magnetic immunoadherence or flow cytometry that uses a cocktail of monoclonal antibodies directed to cell surface markers present on the cells selected. For example, to enrich Tregs, it may be desirable to select regulatory T cells which typically express CD4+, CD25+, CD62Lhi, GITR+, and FoxP3+.

For isolation of a desired population of cells, the concentration of cells and scaffold surface can be varied. In certain embodiments, it may be desirable to significantly decrease the volume in which the FOXP3 disrupting agent and T cells are mixed together (i.e., increase the concentration of cells), to ensure maximum contact of cells and the FOXP3 disrupting agent. For example, in one embodiment, a concentration of 2 billion cells/ml is used. In one embodiment, a concentration of 1 billion cells/ml is used. In a further embodiment, greater than 100 million cells/ml is used. In a further embodiment, a concentration of cells of 10, 15, 20, 25, 30, 35, 40, 45, or 50 million cells/ml is used. In yet another embodiment, a concentration of cells from 75, 80, 85, 90, 95, or 100 million cells/ml is used. In further embodiments, concentrations of 125 or 150 million cells/ml can be used. Using high concentrations can result in increased cell yield, cell activation, and cell expansion. Further, use of high cell concentrations allows more efficient capture of cells that may weakly express target antigens of interest.

In one embodiment, the instant invention may include art-known procedures for sample preparation. For example, T cells may be frozen after the washing step and thawed prior to use. Freezing and subsequent thawing provides a more uniform product by removing granulocytes and to some extent monocytes in the cell population. After the washing step that removes plasma and platelets, the cells may be suspended in a freezing solution. While many freezing solutions and parameters are known in the art and will be useful in this context, one method involves using PBS containing 20% DMSO and 8% human serum albumin, or other suitable cell freezing media containing for example, HESPAN and PLASMALYTE A, the cells then are frozen to −80° C. at a rate of 1° per minute and stored in the vapor phase of a liquid nitrogen storage tank. Other methods of controlled freezing may be used as well as uncontrolled freezing immediately at −20° C. or in liquid nitrogen.

Also contemplated in the context of the invention is the collection of blood samples or leukapheresis product from a subject at a time period prior to when the expanded cells as described herein might be needed. As such, the source of the cells to be expanded can be collected at any time point necessary, and desired cells, such as T cells, isolated and frozen for later use in T cell therapy for any number of diseases or conditions that would benefit from T cell therapy, such as those described herein. In one embodiment a blood sample or a leukapheresis is taken from a generally healthy subject. In certain embodiments, a blood sample or a leukapheresis is taken from a generally healthy subject who is at risk of developing a disease, but who has not yet developed a disease, and the cells of interest are isolated and frozen for later use. In certain embodiments, the T cells may be expanded, frozen, and used at a later time. In certain embodiments, samples are collected from a patient shortly after diagnosis of a particular disease as described herein but prior to any treatments.

In a related embodiment, the instant invention relates to methods for obtaining a polyclonal population of CD4+/FOXP3+ or CD4+/FOXP3− cells. The method comprises contacting the FOXP3 disrupting agent of the invention with a subject's biological sample, thereby activating, and optionally expanding a population of T-cells present within the sample; contacting the T-cells in the sample with a reagent for detection of CD4+ cells; further contacting the T-cells with a reagent for detection of FOXP3+ cells; and isolating a sub-population of detected CD4+/FOXP3+ or CD4+/FOXP3− T-cells from the sample. In these embodiments, the reagent for the detection and/or isolation of CD4+ and/or FOXP3+T− cells is preferably an antibody or antigen-binding fragment thereof which specifically binds to CD4+ and FOXP3 markers.

In yet another embodiment, the present invention relates to methods for obtaining a population of naïve T cells. The method for isolating naïve T cells are known in the art, for example, using commercially available kits such as EasySep™ Human Naïve CD4+ T Cell Isolation Kit of STEMCELL Technologies.

In certain embodiments, the immune cells that have been differentiated/activated may be further expanded. For example, the activated Tregs may be cultured in the presence of certain cytokines, e.g., IL-2, to be further expanded.

Accordingly, in another aspect, the present invention provides immune cells which include the FOXP3 disrupting agent. In some embodiments, the FOXP3 disrupting agent may be present in the immune cells for a period of time that is long enough to induce the immune cells, e.g., naïve T cells, to differentiate into Tregs or to activate the Tregs. In certain embodiments, the immune cells may contain one or more genetic modifications that modulate, e.g., activate, the expression of the FOXP3 gene. Such a genetic modification may be present in the cells after the FOXP3 disrupting agent disappears from the cells or remains in the cell at very low level. Accordingly, the expression of the FOXP3 gene may remain activated even after the FOXP3 disrupting agent stops functioning.

In such context, the genetic modification may be introduced by the site-specific FOXP3 disrupting agent. The genetic modification includes addition, deletion, or substitution of one or more nucleotides to a target sequence, e.g., DNA regions around/proximally upstream of the TSS of FOXP3 gene. The genetic modification may be an epigenetic modification of one or more nucleotides of the target sequence (e.g., methylation/demethylation) or an epigenetic modification of one or more chromatin proteins (e.g., acetylation/deacetylation) at the target sequence, e.g., DNA regions around/proximally upstream of the TSS of FOXP3 gene.

C. In Vivo Methods of the Invention

The in vivo methods of the invention may include administering to a subject an agent, a composition, or cells of the invention.

In one embodiment, immune cells, e.g., naïve T cell or Tregs, are manipulated (e.g., activated) in vivo by providing the FOXP3 disrupting agent of the invention such that the immune cells, e.g., naïve T cells or Tregs, come into contact with the disrupting agent. In order to facilitate the contact, the FOXP3 disrupting agent may be administered in a subject, e.g., subcutaneously or intravenously.

The term “subject,” as used herein refers to an organism, for example, a mammal (e.g., a human, a non-human mammal, a non-human primate, a primate, a laboratory animal, a mouse, a rat, a hamster, a gerbil, a cat, or a dog). In some embodiments a human subject is an adult, adolescent, or pediatric subject. In some embodiments, a subject had a disease or a condition. In some embodiments, the subject is suffering from a disease, disorder or condition, e.g., a disease, disorder or condition that can be treated as provided herein. In some embodiments, a subject is susceptible to a disease, disorder, or condition; in some embodiments, a susceptible subject is predisposed to and/or shows an increased risk (as compared to the average risk observed in a reference subject or population) of developing the disease, disorder or condition. In some embodiments, a subject displays one or more symptoms of a disease, disorder or condition. In some embodiments, a subject does not display a particular symptom (e.g., clinical manifestation of disease) or characteristic of a disease, disorder, or condition. In some embodiments, a subject does not display any symptom or characteristic of a disease, disorder, or condition. In some embodiments, a subject is a patient. In some embodiments, a subject is an individual to whom diagnosis and/or therapy is and/or has been administered.

Subjects that would benefit from the methods of the invention include subjects having an autoimmune disease, a subject at risk of an autoimmune disease, “FOXP3-associated disease,” or a subject at risk of an “FOXP3-associated disease.”

Thus, the present invention further provides methods of treatment of a subject in need thereof. The treatment methods of the invention include administering an agent, a composition, or cells of the invention to a subject, e.g., a subject that would benefit from a modulation of FOXP3 expression, such as a subject having an autoimmune disease or a FOXP3-associated disease, in a therapeutically effective amount. In some embodiments, the methods of the invention include the subject may be administered two site-specific FOX3P disrupting agents (a first and a second agent). The two site specific FOX3P disrupting agents may be present in the same composition, e.g., pharmaceutical composition, e.g., pharmaceutical composition comprising an LNP, or in separate compositions, e.g., pharmaceutical composition, e.g., pharmaceutical composition comprising an LNP. The subject may be administered the first site specific FOX3P disrupting agent at one time and administered the second site specific FOX3P disrupting agent at a second time, or the subject may be administered both agents at the same time.

In addition, the present invention provides methods for preventing at least one symptom in a subject that would benefit from a modulation of FOXP3 expression, such as a subject having an autoimmune disease or a FOXP3-associated disease, by administering to the subject an agent, a composition, or cells of the invention in a prophylactically effective amount.

“Therapeutically effective amount,” as used herein, is intended to include the amount of an agent or composition or cells that, when administered to a patient for treating a subject having an autoimmune disease or a FOXP3-associated disease, is sufficient to effect treatment of the disease (e.g., by diminishing, ameliorating, or maintaining the existing disease or one or more symptoms of disease or its related comorbidities). The “therapeutically effective amount” may vary depending on the agent or composition or cells, how it is administered, the disease and its severity and the history, age, weight, family history, genetic makeup, stage of pathological processes mediated by FOXP3 gene expression, the types of preceding or concomitant treatments, if any, and other individual characteristics of the patient to be treated.

“Prophylactically effective amount,” as used herein, is intended to include the amount of an agent or composition or cells that, when administered to a subject who does not yet experience or display symptoms of a FOXP3-associated disease, but who may be predisposed to a FOXP3-associated disease, is sufficient to prevent or delay the development or progression of the disease or one or more symptoms of the disease for a clinically significant period of time. The “prophylactically effective amount” may vary depending on the agent or composition, how it is administered, the degree of risk of disease, and the history, age, weight, family history, genetic makeup, the types of preceding or concomitant treatments, if any, and other individual characteristics of the patient to be treated.

As used herein, “prevention” or “preventing,” when used in reference to a disease, disorder or condition thereof, that would benefit from an activation in expression of a FOXP3 gene or production of FOXP3 protein, refers to a reduction in the likelihood that a subject will develop a symptom associated with such a disease, disorder, or condition, e.g., a sign or symptom of Tregs or FOXP3 gene dysfunction.

A “therapeutically-effective amount” or “prophylactically effective amount” also includes an amount of an agent or composition or cells that produces some desired local or systemic effect at a reasonable benefit/risk ratio applicable to any treatment. Agents and compositions or cells employed in the methods of the present invention may be administered in a sufficient amount to produce a reasonable benefit/risk ratio applicable to such treatment. In some embodiments, a therapeutically effective amount or prophylactically effect amount is administered in a single dose; in some embodiments, multiple unit doses are required to deliver a therapeutically or prophylactically effective amount.

As used herein, the phrase “symptoms are reduced” may be used when one or more symptoms of a particular disease, disorder or condition is reduced in magnitude (e.g., intensity, severity, etc.) and/or frequency. In some embodiments, a delay in the onset of a particular symptom is considered one form of reducing the frequency of that symptom.

When the subject to be treated is a mammal such as a human, the composition or cells can be administered by any means known in the art including, but not limited to oral, intraperitoneal, or parenteral routes, including intracranial (e.g., intraventricular, intraparenchymal, and intrathecal), intravenous, intramuscular, subcutaneous, transdermal, airway (aerosol), nasal, rectal, and topical (including buccal and sublingual) administration. In certain embodiments, the compositions are administered by intravenous infusion or injection. In certain embodiments, the compositions are administered by subcutaneous injection.

As used herein, the term “FOXP3-associated disease,” includes a disease, disorder or condition that would benefit from a modulation, e.g., an increase, in FOXP3 gene expression, replication, or protein activity, such as an autoimmune disease or a disease that is associated with a Treg dysfunction. Non-limiting examples of FOXP3-associated diseases include autoimmune diseases, for example, IPEX syndrome (IPEX), Type 1 diabetes, Multiple sclerosis, Systemic lupus erythematosus (SLE), Rheumatoid arthritis (RA), Achalasia, Addison's disease, Adult Still's disease, Agammaglobulinemia, Alopecia areata, Amyloidosis, Ankylosing spondylitis, Anti-GBM/Anti-TBM nephritis, Antiphospholipid syndrome, Autoimmune angioedema, Autoimmune dysautonomia, Autoimmune encephalomyelitis, Autoimmune hepatitis, Autoimmune inner ear disease (AIED), Autoimmune myocarditis, Autoimmune oophoritis, Autoimmune orchitis, Autoimmune pancreatitis, Autoimmune retinopathy, Autoimmune urticaria, Axonal & neuronal neuropathy (AMAN), Baló disease, Behcet's disease, Benign mucosal pemphigoid, Bullous pemphigoid, Castleman disease (CD), Celiac disease, Chagas disease, Chronic inflammatory demyelinating polyneuropathy (CIDP), Chronic recurrent multifocal osteomyelitis (CRMO), Churg-Strauss Syndrome (CSS) or Eosinophilic Granulomatosis (EGPA), Cicatricial pemphigoid, Cogan's syndrome, Cold agglutinin disease, Congenital heart block, Coxsackie myocarditis, CREST syndrome, Crohn's disease, Dermatitis herpetiformis, Dermatomyositis, Devic's disease (neuromyelitis optica), Discoid lupus, Dressler's syndrome, Endometriosis, Eosinophilic esophagitis (EoE), Eosinophilic fasciitis, Erythema nodosum, Essential mixed cryoglobulinemia, Evans syndrome, Fibromyalgia, Fibrosing alveolitis, Giant cell arteritis (temporal arteritis), Giant cell myocarditis, Glomerulonephritis, Goodpasture's syndrome, Granulomatosis with Polyangiitis, Graves' disease, Guillain-Barre syndrome, Hashimoto's thyroiditis, Hemolytic anemia, Henoch-Schonlein purpura (HSP), Herpes gestationis or pemphigoid gestationis (PG), Hidradenitis Suppurativa (HS) (Acne Inversa), Hypogammalglobulinemia, IgA Nephropathy, IgG4-related sclerosing disease, Immune thrombocytopenic purpura (ITP), Inclusion body myositis (IBM), Interstitial cystitis (IC), Juvenile arthritis, Juvenile diabetes (Type 1 diabetes), Juvenile myositis (JM), Kawasaki disease, Lambert-Eaton syndrome, Leukocytoclastic vasculitis, Lichen planus, Lichen sclerosus, Ligneous conjunctivitis, Linear IgA disease (LAD), Lupus, Lyme disease chronic, Meniere's disease, Microscopic polyangiitis (MPA), Mixed connective tissue disease (MCTD), Mooren's ulcer, Mucha-Habermann disease, Multifocal Motor Neuropathy (MMN) or MMNCB, Myasthenia gravis, Myositis, Narcolepsy, Neonatal Lupus, Neuromyelitis optica, Neutropenia, Ocular cicatricial pemphigoid, Optic neuritis, Palindromic rheumatism (PR), PANDAS, Paraneoplastic cerebellar degeneration (PCD), Paroxysmal nocturnal hemoglobinuria (PNH), Parry Romberg syndrome, Pars planitis (peripheral uveitis), Parsonage-Turner syndrome, Pemphigus, Peripheral neuropathy, Perivenous encephalomyelitis, Pernicious anemia (PA), POEMS syndrome, Polyarteritis nodosa, Polyglandular syndromes type I, II, III, Polymyalgia rheumatica, Polymyositis, Postmyocardial infarction syndrome, Postpericardiotomy syndrome, Primary biliary cirrhosis, Primary sclerosing cholangitis, Progesterone dermatitis, Psoriasis, Psoriatic arthritis, Pure red cell aplasia (PRCA), Pyoderma gangrenosum, Raynaud's phenomenon, Reactive Arthritis, Reflex sympathetic dystrophy, Relapsing polychondritis, Restless legs syndrome (RLS), Retroperitoneal fibrosis, Rheumatic fever, Sarcoidosis, Schmidt syndrome, Scleritis, Scleroderma, Sjögren's syndrome, Sperm & testicular autoimmunity, Stiff person syndrome (SPS), Subacute bacterial endocarditis (SBE), Susac's syndrome, Sympathetic ophthalmia (SO), Takayasu's arteritis, Temporal arteritis/Giant cell arteritis, Thrombocytopenic purpura (TTP), Tolosa-Hunt syndrome (THS), Transverse myelitis, Ulcerative colitis (UC), Undifferentiated connective tissue disease (UCTD), Uveitis, Vasculitis, Vitiligo, Vogt-Koyanagi-Harada Disease.

In one embodiment, a FOXP3-associated disease is selected from the group consisting of IPEX syndrome (IPEX), Type 1 diabetes, Multiple sclerosis, Systemic lupus erythematosus (SLE), and Rheumatoid arthritis (RA).

Further details regarding signs and symptoms of the various diseases or conditions are provided herein and are well known in the art (see, e.g., ghr.nlm.nih.gov)

Administration of the agents or compositions or cells of the invention according to the methods of the invention may result in a reduction of the severity, signs, symptoms, or markers of a FOXP3-associated disease or disorder in a patient with a FOXP3-associated disease or disorder. By “reduction” in this context is meant a statistically significant decrease in such level. The reduction (absolute reduction or reduction of the difference between the elevated level in the subject and a normal level) can be, for example, at least about 20%, 25%, 30%, 35%, 40%, 45%, 50%, 55%, 60%, 65%, 70%, 75%, 80%, 85%, 90%, or 95%, or to below the level of detection of the assay used.

Administration of the agents or compositions or cells according to the methods of the invention may stably or transiently modulating expression of a target gene, or may stably or transiently increase the amount or the activation level of Tregs. In some embodiments, a modulation of expression persists for at least about 1 hr to about 30 days, or at least about 2 hrs, 6 hrs, 12 hrs, 18 hrs, 24 hrs, 2 days, 3, days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, or longer or any time therebetween. In some other embodiments, a modulation of expression persists for no more than about 30 mins to about 7 days, or no more than about 1 hr, 2 hrs, 3 hrs, 4 hrs, 5 hrs, 6 hrs, 7 hrs, 8 hrs, 9 hrs, 10 hrs, 11 hrs, 12 hrs, 13 hrs, 14 hrs, 15 hrs, 16 hrs, 17 hrs, 18 hrs, 19 hrs, 20 hrs, 21 hrs, 22 hrs, 24 hrs, 36 hrs, 48 hrs, 60 hrs, 72 hrs, 4 days, 5 days, 6 days, 7 days, or any time therebetween. In certain embodiments, the amount of Tregs may increase by about at least 5% to about 10 fold, or at least about 10%, 20%, 30%, 40%, 50%, 60%, 70%, 80%, 90%, 1 fold, 2 fold, 3 fold, 4 fold, 5 fold, 6 fold, 7 fold, 8 fold, 9 fold, 10 fold, or more or any time therebetween. In certain embodiment, the percentage of activated Tregs, e.g., Tregs characterized by increased expression of FOXP3, may increase by about at least 5% to about 10 fold, or at least about 10%, 20%, 30%, 40%, 50%, 60%, 70%, 80%, 90%, 1 fold, 2 fold, 3 fold, 4 fold, 5 fold, 6 fold, 7 fold, 8 fold, 9 fold, 10 fold, or more or any time therebetween. In some embodiments, the expression of FOXP3 in a Tregs, or in a population of Tregs, may increase by about at least 5% to about 10 fold, or at least about 10%, 20%, 30%, 40%, 50%, 60%, 70%, 80%, 90%, 1 fold, 2 fold, 3 fold, 4 fold, 5 fold, 6 fold, 7 fold, 8 fold, 9 fold, 10 fold, or more or any time therebetween.

The agents or compositions or cells may be administered once to the subject or, alternatively, multiple administrations may be performed over a period of time. For example, two, three, four, five, or more administrations may be given to the subject during one treatment or over a period of time. In some embodiments, six, eight, ten, 12, 15 or 20 or more administrations may be given to the subject during one treatment or over a period of time as a treatment regimen.

In some embodiments, administrations may be given as needed, e.g., for as long as symptoms associated with the disease, disorder or condition persist. In some embodiments, repeated administrations may be indicated for the remainder of the subject's life. Treatment periods may vary and could be, e.g., one day, two days, three days, one week, two weeks, one month, two months, three months, six months, a year, or longer.

Efficacy of treatment or prevention of disease can be assessed, for example by measuring disease progression, disease remission, symptom severity, reduction in pain, quality of life, dose of a medication required to sustain a treatment effect, level of a disease marker, or any other measurable parameter appropriate for a given disease being treated or targeted for prevention. It is well within the ability of one skilled in the art to monitor efficacy of treatment or prevention by measuring any one of such parameters, or any combination of parameters. As discussed herein, the specific parameters to be measured depend on the autoimmune disease or FOXP3-associated disease that the subject is suffering from.

Comparisons of the later readings with the initial readings provide a physician an indication of whether the treatment is effective. It is well within the ability of one skilled in the art to monitor efficacy of treatment or prevention by measuring any one of such parameters, or any combination of parameters. In connection with the administration of an agent or composition, “effective against” an autoimmune disease or a FOXP3-associated disorder indicates that administration in a clinically appropriate manner results in a beneficial effect for at least a statistically significant fraction of patients, such as a improvement of symptoms, a cure, a reduction in disease, extension of life, improvement in quality of life, or other effect generally recognized as positive by medical doctors familiar with treating FOXP3-associated disorders.

A treatment or preventive effect is evident when there is a statistically significant improvement in one or more parameters of disease status, or by a failure to worsen or to develop symptoms where they would otherwise be anticipated. As an example, a favorable change of at least 10% in a measurable parameter of disease, and preferably at least 20%, 30%, 40%, 50% or more can be indicative of effective treatment. Efficacy for a given agent or composition can also be judged using an experimental animal model for the given disease as known in the art. When using an experimental animal model, efficacy of treatment is evidenced when a statistically significant reduction in a marker or symptom is observed.

Alternatively, the efficacy can be measured by a reduction in the severity of disease as determined by one skilled in the art of diagnosis based on a clinically accepted disease severity grading scale. Any positive change resulting in e.g., lessening of severity of disease measured using the appropriate scale, represents adequate treatment using an agent or composition as described herein.

As used herein, the terms “treating” or “treatment” refer to a beneficial or desired result including, but not limited to, alleviation or amelioration of one or more signs or symptoms associated with an autoimmune disease or reduction in FOXP3 gene expression or FOXP3 protein production. “Treatment” can also mean prolonging survival as compared to expected survival in the absence of treatment.

D. Combination Methods

The present invention further provides combination methods to activate Treg cells. In certain embodiments, in vitro or ex vivo differentiation or activation of Tregs by FOXP3 activation can be combined with stimulation by TGFβ, for example, by including TGFβ in the growth medium used when culturing cells in vitro or ex vivo, as described herein. TGFβ is an important growth factor in T-cell differentiation and known for triggering FOXP3 activation.

The present invention is next described by means of the following examples. However, the use of these and other examples anywhere in the specification is illustrative only, and in no way limits the scope and meaning of the invention or of any exemplified form. The invention is not limited to any particular preferred embodiments described herein. Many modifications and variations of the invention may be apparent to those skilled in the art and can be made without departing from its spirit and scope. The contents of all references, patents and published patent applications cited throughout this application, including the figures, are incorporated herein by reference.

### EXAMPLES

### Example 1. FOXP3 Activation in Jurkat Cells

This example describes activation of FOXP3 expression in Jurkat cells, as measured by the increase of FOXP3 mRNA level and protein level, using a site-specific FOXP3 disrupting agent, comprising a site-specific FOXP3 targeting moiety which targets a FOXP3 expression control region, i.e., a sgRNA, and an effector comprising dCas9, dCas9 and p300, or dCas9 and VPR.

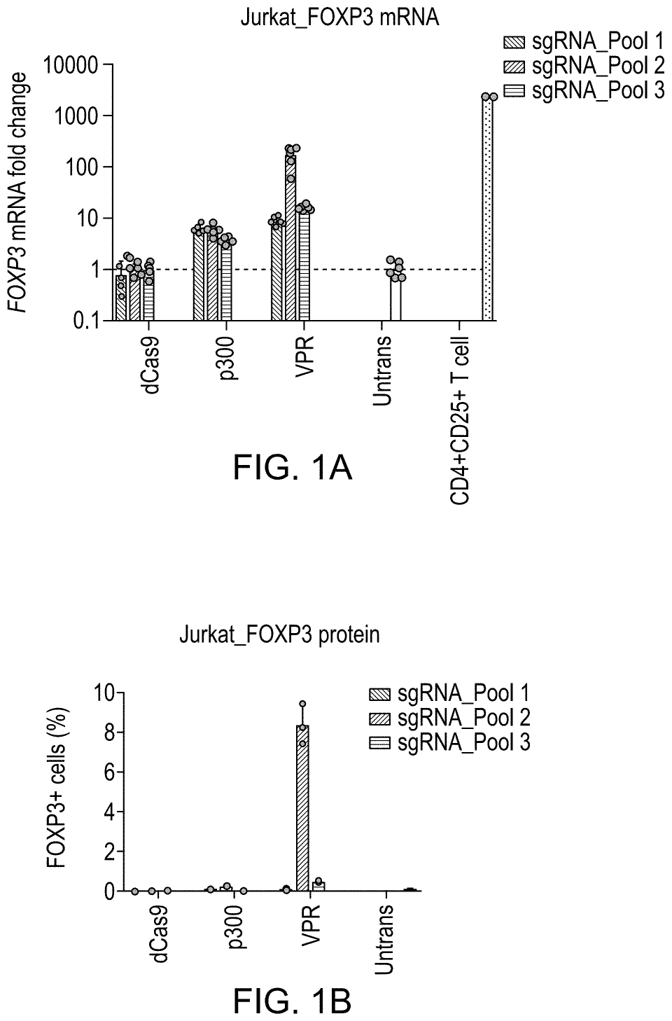

Towards understanding the effect of the activators of the invention on FOXP3 gene expression, Jurkat cells that are human leukemic T-cell line, were transfected with either dCas9- or dCas9-p300- or dCas9-VPR-encoding mRNA along with sgRNA targeting different regions around and upstream of the transcription start site (TSS) with Lipofectamine Messeger Max according to manufacturer's recommendations. Pools of three guide RNAs were used and one pool was found to activate FOXP3 mRNA with both p300 (9 fold) and VPR (100 fold) (FIG. 1A). A pool of sgRNAs was found to induce FOXP3 protein production in combination with a VPR protein. Eight percent (8%) of the cells turned FOXP3 positive when assayed by FACS after treatment with a combination of sgRNAs (Pool2) and mRNA encoding dCas9-VPR (FIG. 1B).

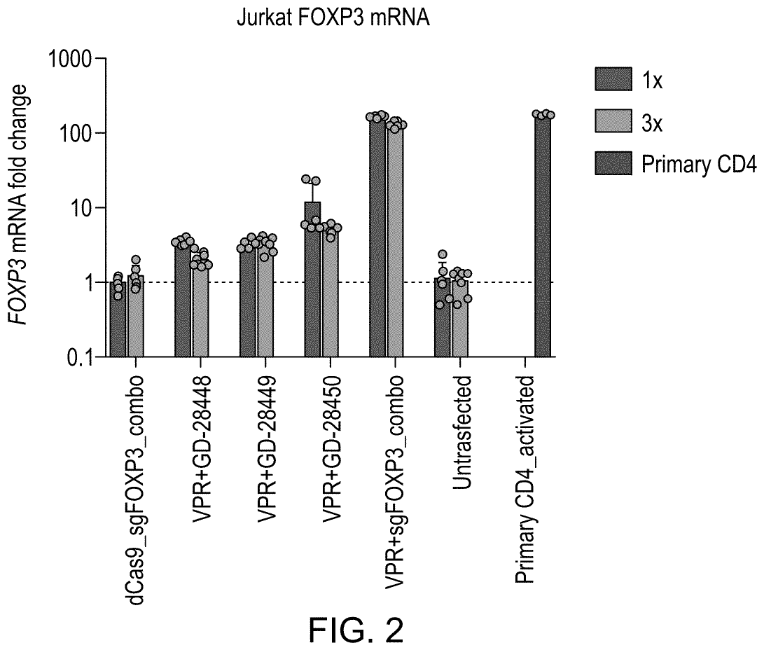

The activation response by VPR can be achieved when using guide pools or individual guides. Without wishing to be bound by theory, it is believed that the activation observed in this experiment is the result of multiple effectors/activators recruiting more of the activation machinery to the target site (FIG. 2). The guide pools used in Examples 1 and 2 are summarized in Table 2.

### Example 2. FOXP3 Activation in Naïve T-Cells

This example describes activation of FOXP3 expression in naïve T-cells, as measured by the increase of FOXP3 mRNA level and protein level, using a site-specific FOXP3 disrupting agent, comprising a site-specific FOXP3 targeting moiety which targets a FOXP3 expression control region, i.e., a sgRNA, and an effector comprising dCas9, dCas9 and p300, or dCas9 and VPR.

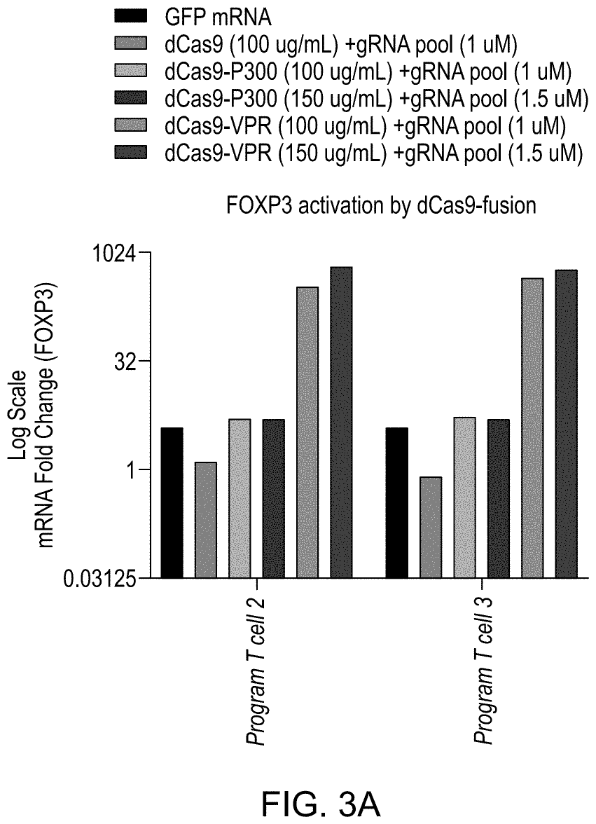

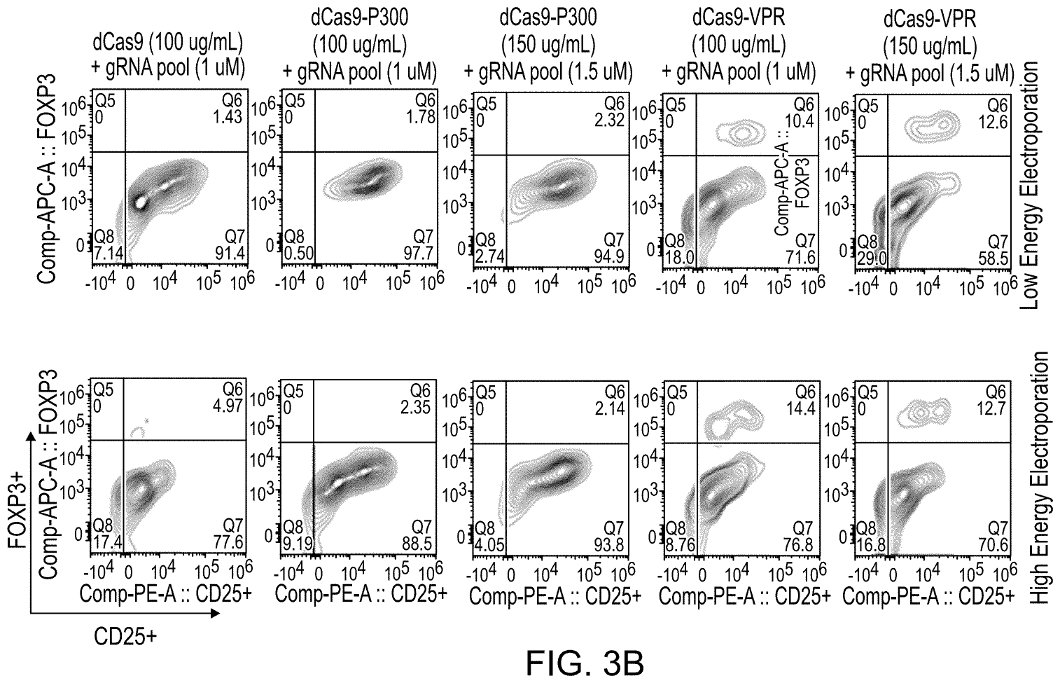

For activation in naïve T-cells, electroporation with MaxCyte ATx using manufacturer's recommended electroporation settings was used to transfect the same mRNA and 3 sgRNA guide combination (Pool 2) as optimized in the Jurkat experiment detailed above to compare the effect of dCas9 alone with dCas9-p300 or dCas9-VPR activator fusions. As in Jurkat cells, VPR was found to elicit the greatest response in terms of FOXP3 mRNA expression (up to 600-fold compared to dCas9 alone) as determined by qPCR with 10 to 14% of the cells determined to be FOXP3+ cells as assayed by FACS analysis (FIG. 3).

TABLE 2
Site-Specific FOXP3 Targeting Moieties - The first 20 nucleotides in each moiety below
comprise the targeting portion of the moiety.

| Identifier | Nucleotide Sequence 5′-3′ |
|---|---|
| GD-28445 | mUs;mCs;mUs;rG;rU;rC;rA;rG;rU;rC;rC;rA;rC;rU;rU;rC;rA;rC;rC;rA;rG;rU;rU;rU;rU;rA;rG;rA;r |
| | G;rC;rU;rA;rG;rA;rA;rA;rU;rA;rG;rC;rA;rA;rG;rU;rU;rA;rA;rA;rA;rU;rA;rA;rG;rG;rC;rU;rA;rG;r |
| | U;rC;rC;rG;rU;rU;rA;rU;rC;rA;rA;rC;rU;rU;rG;rA;rA;rA;rA;rA;rG;rU;rG;rG;rC;rA;rC;rC;rG;rA;rG |
| | ;rU;rC;rG;rG;rU;rG;rC;rUs;mUs;mUs;mU (SEQ ID NO: 96) |
| | TCTGTCAGTCCACTTCACCAGTTTTAGAGCTAGAAATAGCAAGTTAAAATAAGGCTAGT |
| | CCGTTATCAACTTGAAAAAGTGGCACCGAGTCGGTGCTTTT (SEQ ID NO: 97) |
| GD-28446 | mGs;mUs;mGs;rA;rC;rA;rG;rU;rU;rU;rC;rC;rC;rA;rC;rA;rA;rG;rC;rC;rG;rU;rU;rU;rU;rA;rG;rA;r |
| | G;rC;rU;rA;rG;rA;rA;rA;rU;rA;rG;rC;rA;rA;rG;rU;rU;rA;rA;rA;rA;rU;rA;rA;rG;rG;rC;rU;rA;rG;r |
| | U;rC;rC;rG;rU;rU;rA;rU;rC;rA;rA;rC;rU;rU;rG;rA;rA;rA;rA;rA;rG;rU;rG;rG;rC;rA;rC;rC;rG;rA;rG |
| | ;rU;rC;rG;rG;rU;rG;rC;rUs;mUs;mUs;mU (SEQ ID NO: 98) |
| | GTGACAGTTTCCCACAAGCCGTTTTAGAGCTAGAAATAGCAAGTTAAAATAAGGCTAGT |
| | CCGTTATCAACTTGAAAAAGTGGCACCGAGTCGGTGCTTTT (SEQ ID NO: 99) |
| GD-28447 | mAs;mAs;mAs;rA;rA;rC;rC;rA;rC;rG;rC;rU;rG;rU;rA;rC;rG;rG;rU;rG;rG;rU;rU;rU;rU;rA;rG;rA;r |
| | G;rC;rU;rA;rG;rA;rA;rA;rU;rA;rG;rC;rA;rA;rG;rU;rU;rA;rA;rA;rA;rU;rA;rA;rG;rG;rC;rU;rA;rG;r |
| | U;rC;rC;rG;rU;rU;rA;rU;rC;rA;rA;rC;rU;rU;rG;rA;rA;rA;rA;rA;rG;rU;rG;rG;rC;rA;rC;rC;rG;rA;rG |
| | ;rU;rC;rG;rG;rU;rG;rC;rUs;mUs;mUs;mU (SEQ ID NO: 100) |
| | AAAAACCACGCTGTACGGTGGTTTTAGAGCTAGAAATAGCAAGTTAAAATAAGGCTAG |
| | TCCGTTATCAACTTGAAAAAGTGGCACCGAGTCGGTGCTTTT' (SEQ ID NO: 101) |
| GD-28448 | mUs;mGs;mUs;rG;rU;rG;rC;rG;rC;rU;rG;rA;rU;rA;rA;rU;rC;rA;rC;rG;rG;rU;rU;rU;rU;rA;rG;rA;r |
| | G;rC;rU;rA;rG;rA;rA;rA;rU;rA;rG;rC;rA;rA;rG;rU;rU;rA;rA;rA;rA;rU;rA;rA;rG;rG;rC;rU;rA;rG;r |
| | U;rC;rC;rG;rU;rU;rA;rU;rC;rA;rA;rC;rU;rU;rG;rA;rA;rA;rA;rA;rG;rU;rG;rG;rC;rA;rC;rC;rG;rA;rG |
| | ;rU;rC;rG;rG;rU;rG;rC;rUs;mUs;mUs;mU (SEQ ID NO: 102) |
| | TGTGTGCGCTGATAATCACGGTTTTAGAGCTAGAAATAGCAAGTTAAAATAAGGCTAGT |
| | CCGTTATCAACTTGAAAAAGTGGCACCGAGTCGGTGCTTTT (SEQ ID NO: 103) |
| GD-28449 | mUs;mAs;mAs;rG;rU;rC;rU;rC;rA;rU;rA;rA;rU;rC;rA;rA;rG;rA;rA;rA;rG;rU;rU;rU;rU;rA;rG;rA;r |
| | G;rC;rU;rA;rG;rA;rA;rA;rU;rA;rG;rC;rA;rA;rG;rU;rU;rA;rA;rA;rA;rU;rA;rA;rG;rG;rC;rU;rA;rG;r |
| | U;rC;rC;rG;rU;rU;rA;rU;rC;rA;rA;rC;rU;rU;rG;rA;rA;rA;rA;rA;rG;rU;rG;rG;rC;rA;rC;rC;rG;rA;rG |
| | ;rU;rC;rG;rG;rU;rG;rC;rUs;mUs;mUs;mU (SEQ ID NO: 104) |
| | TAAGTCTCATAATCAAGAAAGTTTTAGAGCTAGAAATAGCAAGTTAAAATAAGGCTAG |
| | TCCGTTATCAACTTGAAAAAGTGGCACCGAGTCGGTGCTTTT (SEQ ID NO: 105) |
| GD-28450 | mUs;mAs;mUs;rU;rU;rU;rC;rA;rG;rA;rU;rG;rA;rC;rU;rC;rG;rU;rA;rA;rG;rU;rU;rU;rU;rA;rG;rA;r |
| | G;rC;rU;rA;rG;rA;rA;rA;rU;rA;rG;rC;rA;rA;rG;rU;rU;rA;rA;rA;rA;rU;rA;rA;rG;rG;rC;rU;rA;rG;r |
| | U;rC;rC;rG;rU;rU;rA;rU;rC;rA;rA;rC;rU;rU;rG;rA;rA;rA;rA;rA;rG;rU;rG;rG;rC;rA;rC;rC;rG;rA;rG |
| | ;rU;rC;rG;rG;rU;rG;rC;rUs;mUs;mUs;mU (SEQ ID NO: 106) |
| | TATTTTCAGATGACTCGTAAGTTTTAGAGCTAGAAATAGCAAGTTAAAATAAGGCTAGT |
| | CCGTTATCAACTTGAAAAAGTGGCACCGAGTCGGTGCTTTT (SEQ ID NO: 107) |
| GD-28451 | mUs;mAs;mAs;rA;rU;rC;rA;rC;rA;rG;rG;rG;rC;rC;rA;rA;rC;rC;rC;rG;rG;rU;rU;rU;rU;rA;rG;rA;r |
| | G;rC;rU;rA;rG;rA;rA;rA;rU;rA;rG;rC;rA;rA;rG;rU;rU;rA;rA;rA;rA;rU;rA;rA;rG;rG;rC;rU;rA;rG;r |
| | U;rC;rC;rG;rU;rU;rA;rU;rC;rA;rA;rC;rU;rU;rG;rA;rA;rA;rA;rA;rG;rU;rG;rG;rC;rA;rC;rC;rG;rA;rG |
| | ;rU;rC;rG;rG;rU;rG;rC;rUs;mUs;mUs;mU (SEQ ID NO: 108) |
| | TAAATCACAGGGCCAACCCGGTTTTAGAGCTAGAAATAGCAAGTTAAAATAAGGCTAG |
| | TCCGTTATCAACTTGAAAAAGTGGCACCGAGTCGGTGCTTTT (SEQ ID NO: 109) |
| GD-28452 | mGs;mAs;mCs;rA;rC;rC;rA;rU;rU;rC;rU;rG;rU;rG;rA;rG;rU;rG;rA;rG;rG;rU;rU;rU;rU;rA;rG;rA;r |
| | G;rC;rU;rA;rG;rA;rA;rA;rU;rA;rG;rC;rA;rA;rG;rU;rU;rA;rA;rA;rA;rU;rA;rA;rG;rG;rC;rU;rA;rG;r |
| | U;rC;rC;rG;rU;rU;rA;rU;rC;rA;rA;rC;rU;rU;rG;rA;rA;rA;rA;rA;rG;rU;rG;rG;rC;rA;rC;rC;rG;rA;rG |
| | ;rU;rC;rG;rG;rU;rG;rC;rUs;mUs;mUs;mU (SEQ ID NO: 110) |
| | GACACCATTCTGTGAGTGAGGTTTTAGAGCTAGAAATAGCAAGTTAAAATAAGGCTAG |
| | TCCGTTATCAACTTGAAAAAGTGGCACCGAGTCGGTGCTTTT (SEQ ID NO: 111) |
| GD-28453 | mAs;mGs;mAs;rA;rU;rC;rU;rG;rA;rA;rG;rC;rU;rC;rU;rA;rU;rG;rU;rG;rG;rU;rU;rU;rU;rA;rG;rA;r |
| | G;rC;rU;rA;rG;rA;rA;rA;rU;rA;rG;rC;rA;rA;rG;rU;rU;rA;rA;rA;rA;rU;rA;rA;rG;rG;rC;rU;rA;rG;r |
| | U;rC;rC;rG;rU;rU;rA;rU;rC;rA;rA;rC;rU;rU;rG;rA;rA;rA;rA;rA;rG;rU;rG;rG;rC;rA;rC;rC;rG;rA;rG |
| | ;rU;rC;rG;rG;rU;rG;rC;rUs;mUs;mUs;mU (SEQ ID NO: 112) |
| | AGAATCTGAAGCTCTATGTGGTTTTAGAGCTAGAAATAGCAAGTTAAAATAAGGCTAGT |
| | CCGTTATCAACTTGAAAAAGTGGCACCGAGTCGGTGCTTTT (SEQ ID NO: 113) |

TABLE 3
Site-Specific FOXP3 Targeting Moieties - Nucleotide Sequences of FOXP3 Guides
Used in Examples 1 and 2 and Complementary Target Sequence in the Genome

| | Effector | Guide NT sequence (20 nt | Complementary target sequence |
|---|---|---|---|
| Pool# | ID | targeting region only)* 5′-3′ | 5′→3′ |
| 1 | GD-28445 | TCTGTCAGTCCACTTCACCA (SEQ | TGGTGAAGTGGACTGACAGA |
| | | ID NO: 114) | (SEQ ID NO: 115) |
| | GD-28446 | GTGACAGTTTCCCACAAGCC (SEQ | GGCTTGTGGGAAACTGTCAC (SEQ |
| | | ID NO: 116) | ID NO: 117) |
| | GD-28447 | AAAAACCACGCTGTACGGTG | CACCGTACAGCGTGGTTTTT (SEQ |
| | | (SEQ ID NO: 118) | ID NO: 119) |
| 2** | GD-28448 | TGTGTGCGCTGATAATCACG(SEQ | CGTGATTATCAGCGCACACA (SEQ |
| | | ID NO: 120) | ID NO: 121) |
| | GD-28449 | TAAGTCTCATAATCAAGAAA (SEQ | TTTCTTGATTATGAGACTTA (SEQ |
| | | ID NO: 122) | ID NO: 123) |
| | GD-28450 | TATTTTCAGATGACTCGTAA (SEQ | TTACGAGTCATCTGAAAATA (SEQ |
| | | ID NO: 124) | ID NO: 125) |
| 3 | GD-28451 | TAAATCACAGGGCCAACCCG | CGGGTTGGCCCTGTGATTTA (SEQ |
| | | (SEQ ID NO: 126) | ID NO: 127) |
| | GD-28452 | GACACCATTCTGTGAGTGAG (SEQ | CTCACTCACAGAATGGTGTC (SEQ |
| | | ID NO: 128) | ID NO: 129) |
| | GD-28453 | AGAATCTGAAGCTCTATGTG (SEQ | CACATAGAGCTTCAGATTCT (SEQ |
| | | ID NO: 130) | ID NO: 131) |

Note:
*All single guides were 100 nucleotide long. The indicated 20mer targeting region was part of the SpCas9 PAM single guide RNA having the following 80 nt sequence for dCas9 binding: 5’-GTTTTAGAGCTAGAAATAGCAAGTTAAAATAAGGCTAGTCCGTTATCAACTTGAAAAAGTGGCACCGAGTCGGTGCTTTT-3′ (SEQ ID NO: 132)
#Pool numbers are the same as those in FIGS. 1A and IB.
**Pool 2 strongly induced upregulation response in Jurkat cells and was successfully used to activate FOXP3 in naïve T-cells.

TABLE 4
Site-Specific FOXP3 Targeting Moieties

| Effector | | | | | PAM | Length |
|---|---|---|---|---|---|---|
| ID | Target Sequence | Strand | Genome | Species | Sequence | (NTs) |
| GD- | TCTGTCAGTCCACTTCACCA | − | GRCh37: | Human | AGG | 100 |
| 28445 | (SEQID NO: 133) | | chrX:49121122-49121144 | | | |
| GD- | GTGACAGTTTCCCACAAGCC | − | GRCh37: | Human | AGG | 100 |
| 28446 | (SEQID NO: 134) | | chrX:49121155-49121177 | | | |
| GD- | AAAAACCACGCTGTACGGTG | + | GRCh37: | Human | TGG | 100 |
| 28447 | (SEQID NO: 135) | | chrX:49121212-49121234 | | | |
| GD- | TGTGTGCGCTGATAATCACG | + | GRCh37: | Human | GGG | 100 |
| 28448 | (SEQID NO: 136) | | chrX:49121282-49121304 | | | |
| GD- | TAAGTCTCATAATCAAGAAA | − | GRCh37: | Human | AGG | 100 |
| 28449 | (SEQID NO: 137) | | chrX:49121352-49121374 | | | |
| GD- | TATTTTCAGATGACTCGTAA | − | GRCh37: | Human | AGG | 100 |
| 28450 | (SEQID NO: 138) | | chrX:49121411-49121433 | | | |
| GD- | TAAATCACAGGGCCAACCCG | + | GRCh37: | Human | AGG | 100 |
| 28451 | (SEQID NO: 139) | | chrX:49121561-49121583 | | | |
| GD- | GACACCATTCTGTGAGTGAG | + | GRCh37: | Human | AGG | 100 |
| 28452 | (SEQ ID NO: 140) | | chrX:49121592-49121614 | | | |
| GD- | AGAATCTGAAGCTCTATGTG | + | GRCh37: | Human | TGG | 100 |
| 28453 | (SEQ ID NO: 141) | | chrX:49121643-49121665 | | | |

TABLE 5
Abbreviations of nucleotide monomers used in nucleic
acid sequence representation. It will be understood
that these monomers, when present in an oligonucleotide,
are mutually linked by 5′-3′-phosphodiester bonds.

| Abbreviation | Nucleotide(s) |
|---|---|
| A | Adenosine-3′-phosphate |
| As | adenosine-3′-phosphorothioate |
| C | cytidine-3′-phosphate |
| Cs | cytidine-3′-phosphorothioate |
| G | guanosine-3′-phosphate |
| Gs | guanosine-3′-phosphorothioate |
| U | Uridine-3′-phosphate |
| Us | uridine -3′-phosphorothioate |
| N | any nucleotide, modified or unmodified |
| mA | 2′-O-methyladenosine-3′-phosphate |
| mAs | 2′-O-methyladenosine-3′-phosphorothioate |
| mC | 2′-O-methylcytidine-3′-phosphate |
| mCs | 2′-O-methylcytidine-3′-phosphorothioate |
| mG | 2′-O-methylguanosine-3′-phosphate |
| mGs | 2′-O-methylguanosine-3′-phosphorothioate |
| mU | 2′-O-methyluridine-3′-phosphate |
| mUs | 2′-O-methyluridine-3′-phosphorothioate |
| s | phosphorothioate linkage |
| r | ribonucleotide |

1. A site-specific forkhead box P3(FOXP3) disrupting agent, comprising a site-specific FOXP3 targeting moiety which targets a FOXP3 expression control region.

2. The site-specific FOXP3 disrupting agent of claim 1,
(a) wherein the site-specific FOXP3 targeting moiety comprises a polymeric molecule; optionally a polyamide or a polynucleotide; optionally wherein the polymeric molecule comprises a peptide nucleic acid (PNA);
(b) wherein the expression control region comprises a region upstream of a FOXP3 transcription start site (TSS);
(c) wherein the expression control region comprises one or more FOXP3-associated anchor sequences within an anchor sequence-mediated conjunction comprising a first and a second FOXP3-associated anchor sequence;
optionally, wherein the anchor sequence comprises a CCCTC-binding factor (CTCF) binding motif;
optionally, wherein the anchor sequence-mediated conjunction comprises one or more transcriptional control elements internal to the conjunction, or one or more transcriptional control elements external to the conjunction;
optionally, wherein the first and/or the second anchor sequence is located within about 500 kb, or within about 300 kb, or within about 10 kb, of the transcriptional control element;
(d) wherein the expression control region comprises a FOXP3-specific transcriptional control element, optionally, wherein the transcriptional control element comprises a FOXP3 promoter, a transcriptional enhancer, or a transcriptional repressor; and/or
(e) wherein the disrupting agent comprises a modification.

3-16. (canceled)

17. The site-specific FOXP3 disrupting agent of claim 1, wherein the FOXP3 targeting moiety comprises
(a) a nucleotide sequence having at least 85% nucleotide identity to the entire nucleotide sequence of any one of the nucleotide sequences in Table 2;
(b) a first nucleotide sequence having at least 85% nucleotide identity to the entire nucleotide sequence of GD-28448, a second nucleotide sequence having at least 85% nucleotide identity to the entire nucleotide sequence of GD-28449, and a third nucleotide sequence having at least 85% nucleotide identity to the entire nucleotide sequence of GD-28450; and/or
(c) a polymeric molecule comprises a polynucleotide encoding a DNA-binding domain, or fragment thereof, of a zinc finger polypeptide (ZNF) or a transcription activator-like effector (TALE) polypeptide that specifically binds to the FOXP3 expression control region; optionally, wherein the DNA-binding domain of the TALE or ZNF polypeptide comprises an amino acid sequence having at least about 85% amino acid identity to the entire amino acid sequence of any one of the amino acid sequences listed in Table 1B.

18-22. (canceled)

23. A vector comprising the site-specific FOXP3 disrupting agent of claim 1, optionally, wherein the vector is a viral expression vector.

24. (canceled)

25. A cell comprising the site-specific FOXP3 disrupting agent of claim 1, optionally, wherein the cell is an immune cell.

26. (canceled)

27. (canceled)

28. The site-specific FOXP3 disrupting agent of claim 1, wherein the site-specific FOXP3 disrupting agent is present in a composition; optionally, wherein the composition comprises a pharmaceutical composition; optionally,
(a) wherein the pharmaceutical composition comprises a lipid formulation, wherein the lipid formulation comprises one or more cationic lipids, one or more non-cationic lipids, one or more cholesterol-based lipids, or one or more PEG-modified lipids, or combinations of any of the foregoing; and/or
(b) wherein the pharmaceutical composition comprises a lipid nanoparticle.

29-32. (canceled)

33. A site-specific FOXP3 disrupting agent, comprising a nucleic acid molecule encoding a fusion protein, the fusion protein comprising a site-specific FOXP3 targeting moiety which targets a FOXP3 expression control region and an effector molecule.

34. The site-specific FOXP3 disrupting agent of claim 33,
(a) wherein the site-specific FOXP3 targeting moiety comprises a polynucleotide encoding a DNA-binding domain, or fragment thereof, of a zinc finger polypeptide (ZNF) or a transcription activator-like effector (TALE) polypeptide that specifically binds to the FOXP3 expression control region; optionally, wherein the DNA-binding domain of the TALE or zinc finger polypeptide comprises an amino acid sequence having at least 85% amino acid identity to the entire amino acid sequence of an amino acid sequence selected from the amino acid sequences listed in Table 1B;
(b) wherein the effector molecule comprises a nucleic acid molecule encoding a polypeptide;
(c) wherein the fusion protein comprises a peptide-nucleic acid fusion;
(d) wherein the effector is selected from the group consisting of a nuclease, a physical blocker, an epigenetic recruiter, and an epigenetic CpG modifier, and combinations of any of the foregoing;
(e) wherein the effector comprises a CRISPR associated protein (Cas) polypeptide or nucleic acid molecule encoding the Cas polypeptide; optionally, wherein the Cas polypeptide is an enzymatically inactive Cas polypeptide, or further comprises a catalytically active domain of human exonuclease 1 (hEXO1);
(f) wherein the epigenetic recruiter comprises a transcriptional enhancer or a transcriptional repressor;
(g) wherein the epigenetic CpG modifier comprises a DNA methylase, a DNA demethylase, a histone modifying agent, a histone transacetylase, or a histone deacetylase;
(h) wherein the effector molecule comprises a zinc finger polypeptide; and/or
(i) wherein the effector molecule comprises a Transcription activator-like effector nuclease (TALEN) polypeptide.

35-42. (canceled)

43. The site-specific FOXP3 disrupting agent of claim 34,
(a) wherein the transcriptional enhancer is a VPR (VP64-p65-Rta); optionally,
wherein the VPR comprises an amino acid sequence having at least about 85% amino acid identity to the entire amino acid sequence of DALDDFDLDMLGSDALDDFDLDMLGSDALDDFDLDMLGSDALDDFDLDMLSGGPKKKRKVGSQY LPDTDDRHRIEEKRKRTYETFKSIMKKSPFSGPTDPRPPPRRIAVPSRSSASVPKPAPQPYPFTSSLSTIN YDEFPTMVFPSGQISQASALAPAPPQVLPQAPAPAPAPAMVSALAQAPAPVPVLAPGPPQAVAPPAP KPTQAGEGTLSEALLQLQFDDEDLGALLGNSTDPAVFTDLASVDNSEFQQLLNQGIPVAPHTTEPML MEYPEAITRLVTGAQRPPDPAPAPLGAPGLPNGLLSGDEDFSSIADMDFSALLGSGSGSRDSREGMFL PKPEAGSAISDVFEGREVCQPKRLRPFHPPGSPWANRPLPASLAPTPTGPVHEPVGSLTPAPVPQPLDP APAVTPEASHLLEDPDEETSQAVKALREMADTVIPQKEEAAICGQMDLSHPPPRGHLDELTTTLESM TEDLNLDSPLTPELNEILDTFLNDECLLHAMHISTGLSIFDTSLF (SEQ ID NO: 64); and/or,
optionally, wherein the transcriptional enhancer comprises two, three, four, or five VPRs; and/or
(b) wherein the transcriptional enhancer is a p300; optionally,
wherein the p300 comprises an amino acid sequence having at least about 85% identity to the entire amino acid sequence of
(SEQ ID NO: 65)
IFKPEELRQALMPTLEALYRQDPESLPFRQPVDPQLLGIPDYFDIVKSPM
DLSTIKRKLDTGQYQEPWQYVDDIWLMFNNAWLYNRKTSRVYKYCSKLSE
VFEQEIDPVMQSLGYCCGRKLEFSPQTLCCYGKQLCTIPRDATYYSYQNR
YHFCEKCFNEIQGESVSLGDDPSQPQTTINKEQFSKRKNDTLDPELFVEC
TECGRKMHQICVLHHEIIWPAGFVCDGCLKKSARTRKENKFSAKRLPSTR
LGTFLENRVNDFLRRQNHPESGEVTVRVVHASDKTVEVKPGMKARFVDSG
EMAESFPYRTKALFAFEEIDGVDLCFFGMHVQEYGSDCPPPNQRRVYISY
LDSVHFFRPKCLRTAVYHEILIGYLEYVKKLGYTTGHIWACPPSEGDDYI
FHCHPPDQKIPKPKRLQEWYKKMLDKAVSERIVHDYKDIFKQATEDRLTS
AKELPYFEGDFWPNVLEESIKELEQEEEERKREENTSNESTDVTKGDSKN
AKKKNNKKTSKNKSSLSRGNKKKPGMPNVSNDLSQKLYATMEKHKEVFFV
IRLIAGPAANSLPPIVDPDPLIPCDLMDGRDAFLTLARDKHLEFSSLRRA
QWSTMCMLVELHTQSQD

44-50. (canceled)

51. The site-specific FOXP3 disrupting agent of claim 33, further comprising a second nucleic acid molecule encoding a second fusion protein, wherein the second fusion comprises a second site-specific FOXP3 targeting moiety which targets a second FOXP3 expression control region and a second effector molecule, wherein the second FOXP3 expression control region is different than the FOXP3 expression control region; optionally,
(a) wherein the second effector is different than the effector;
(b) wherein the second effector is the same as the effector;
(c) wherein the fusion protein and the second fusion protein are operably linked;
(d) wherein the fusion protein and the second fusion protein comprise an amino acid sequence that has at least about 85% amino acid sequence identity to the entire amino acid sequence of a polypeptide selected from the group consisting of dCas-P300 (SEQ ID NO: 10) and dCas-VPR (SEQ ID NO: 11); and/or
(e) wherein the fusion protein is encoded by a polynucleotide comprising a nucleotide sequence that has at least about 85% amino acid sequence identity to the entire nucleotide sequence of a polynucleotide selected from the group consisting of dCas-P300 mRNA (SEQ ID NO: 7) and dCas-VPR mRNA (SEQ ID NO: 8).

52-56. (canceled)

57. A site-specific FOXP3 disrupting agent, comprising
(a) a nucleic acid molecule encoding a fusion protein, wherein the fusion protein comprises an amino acid sequence having at least about 85% amino acid identity to the entire amino acid sequence of a polypeptide selected from the group consisting of dCas-P300 (SEQ ID NO: 10) and dCas-VPR (SEQ ID NO: 11;
(b) a polynucleotide encoding the amino acid sequence of dCas-P300
comprising the amino acid sequence of
(SEQ ID NO: 10)
MAPKKKRKVGIHGVPAADKKYSIGLAIGTNSVGWAVITDEYKVPSKKFKVLGNTDRHSIKKNLIGA
LLFDSGETAEATRLKRTARRRYTRRKNRICYLQEIFSNEMAKVDDSFFHRLEESFLVEEDKKHERHPI
FGNIVDEVAYHEKYPTIYHLRKKLVDSTDKADLRLIYLALAHMIKFRGHFLIEGDLNPDNSDVDKLFI
QLVQTYNQLFEENPINASGVDAKAILSARLSKSRRLENLIAQLPGEKKNGLFGNLIALSLGLTPNFKS
NFDLAEDAKLQLSKDTYDDDLDNLLAQIGDQYADLFLAAKNLSDAILLSDILRVNTEITKAPLSASM
IKRYDEHHQDLTLLKALVRQQLPEKYKEIFFDQSKNGYAGYIDGGASQEEFYKFIKPILEKMDGTEE
LLVKLNREDLLRKQRTFDNGSIPHQIHLGELHAILRRQEDFYPFLKDNREKIEKILTFRIPYYVGPLAR
GNSRFAWMTRKSEETITPWNFEEVVDKGASAQSFIERMTNFDKNLPNEKVLPKHSLLYEYFTVYNE
LTKVKYVTEGMRKPAFLSGEQKKAIVDLLFKTNRKVTVKQLKEDYFKKIECFDSVEISGVEDRFNAS
LGTYHDLLKIIKDKDFLDNEENEDILEDIVLTLTLFEDREMIEERLKTYAHLFDDKVMKQLKRRRYT
GWGRLSRKLINGIRDKQSGKTILDFLKSDGFANRNFMQLIHDDSLTFKEDIQKAQVSGQGDSLHEHI
ANLAGSPAIKKGILQTVKVVDELVKVMGRHKPENIVIEMARENQTTQKGQKNSRERMKRIEEGIKE
LGSQILKEHPVENTQLQNEKLYLYYLQNGRDMYVDQELDINRLSDYDVAAIVPQSFLKDDSIDNKV
LTRSDKARGKSDNVPSEEVVKKMKNYWRQLLNAKLITQRKFDNLTKAERGGLSELDKAGFIKRQL
VETRQITKHVAQILDSRMNTKYDENDKLIREVKVITLKSKLVSDFRKDFQFYKVREINNYHHAHDA
YLNAVVGTALIKKYPKLESEFVYGDYKVYDVRKMIAKSEQEIGKATAKYFFYSNIMNFFKTEITLAN
GEIRKRPLIETNGETGEIVWDKGRDFATVRKVLSMPQVNIVKKTEVQTGGFSKESILPKRNSDKLIAR
KKDWDPKKYGGFDSPTVAYSVLVVAKVEKGKSKKLKSVKELLGITIMERSSFEKNPIDFLEAKGYK
EVKKDLIIKLPKYSLFELENGRKRMLASAGELQKGNELALPSKYVNFLYLASHYEKLKGSPEDNEQK
QLFVEQHKHYLDEIIEQISEFSKRVILADANLDKVLSAYNKHRDKPIREQAENIIHLFTLTNLGAPAAF
KYFDTTIDRKRYTSTKEVLDATLIHQSITGLYETRIDLSQLGGDKRPAATKKAGQAKKKKGRAIFKP
EELRQALMPTLEALYRQDPESLPFRQPVDPQLLGIPDYFDIVKSPMDLSTIKRKLDTGQYQEPWQYV
DDIWLMFNNAWLYNRKTSRVYKYCSKLSEVFEQEIDPVMQSLGYCCGRKLEFSPQTLCCYGKQLC
TIPRDATYYSYQNRYHFCEKCFNEIQGESVSLGDDPSQPQTTINKEQFSKRKNDTLDPELFVECTECG
RKMHQICVLHHEIIWPAGFVCDGCLKKSARTRKENKFSAKRLPSTRLGTFLENRVNDFLRRQNHPES
GEVTVRVVHASDKTVEVKPGMKARFVDSGEMAESFPYRTKALFAFEEIDGVDLCFFGMHVQEYGS
DCPPPNQRRVYISYLDSVHFFRPKCLRTAVYHEILIGYLEYVKKLGYTTGHIWACPPSEGDDYIFHCH
PPDQKIPKPKRLQEWYKKMLDKAVSERIVHDYKDIFKQATEDRLTSAKELPYFEGDFWPNVLEESIK
ELEQEEEERKREENTSNESTDVTKGDSKNAKKKNNKKTSKNKSSLSRGNKKKPGMPNVSNDLSQK
LYATMEKHKEVFFVIRLIAGPAANSLPPIVDPDPLIPCDLMDGRDAFLTLARDKHLEFSSLRRAQWST
MCMLVELHTQSQDSGGKRPAATKKAGQAKKKKGSYPYDVPDYA;
and/or
(c) a polynucleotide encoding the amino acid sequence of dCas-VPR
comprising the amino acid sequence of
(SEQ ID NO: 11)
MAPKKKRKVGIHGVPAADKKYSIGLAIGTNSVGWAVITDEYKVPSKKFKVLGNTDRHSIKKNLIGA
LLFDSGETAEATRLKRTARRRYTRRKNRICYLQEIFSNEMAKVDDSFFHRLEESFLVEEDKKHERHPI
FGNIVDEVAYHEKYPTIYHLRKKLVDSTDKADLRLIYLALAHMIKFRGHFLIEGDLNPDNSDVDKLFI
QLVQTYNQLFEENPINASGVDAKAILSARLSKSRRLENLIAQLPGEKKNGLFGNLIALSLGLTPNFKS
NFDLAEDAKLQLSKDTYDDDLDNLLAQIGDQYADLFLAAKNLSDAILLSDILRVNTEITKAPLSASM
IKRYDEHHQDLTLLKALVRQQLPEKYKEIFFDQSKNGYAGYIDGGASQEEFYKFIKPILEKMDGTEE
LLVKLNREDLLRKQRTFDNGSIPHQIHLGELHAILRRQEDFYPFLKDNREKIEKILTFRIPYYVGPLAR
GNSRFAWMTRKSEETITPWNFEEVVDKGASAQSFIERMTNFDKNLPNEKVLPKHSLLYEYFTVYNE
LTKVKYVTEGMRKPAFLSGEQKKAIVDLLFKTNRKVTVKQLKEDYFKKIECFDSVEISGVEDRFNAS
LGTYHDLLKIIKDKDFLDNEENEDILEDIVLTLTLFEDREMIEERLKTYAHLFDDKVMKQLKRRRYT
GWGRLSRKLINGIRDKQSGKTILDFLKSDGFANRNFMQLIHDDSLTFKEDIQKAQVSGQGDSLHEHI
ANLAGSPAIKKGILQTVKVVDELVKVMGRHKPENIVIEMARENQTTQKGQKNSRERMKRIEEGIKE
LGSQILKEHPVENTQLQNEKLYLYYLQNGRDMYVDQELDINRLSDYDVAAIVPQSFLKDDSIDNKV
LTRSDKARGKSDNVPSEEVVKKMKNYWRQLLNAKLITQRKFDNLTKAERGGLSELDKAGFIKRQL
VETRQITKHVAQILDSRMNTKYDENDKLIREVKVITLKSKLVSDFRKDFQFYKVREINNYHHAHDA
YLNAVVGTALIKKYPKLESEFVYGDYKVYDVRKMIAKSEQEIGKATAKYFFYSNIMNFFKTEITLAN
GEIRKRPLIETNGETGEIVWDKGRDFATVRKVLSMPQVNIVKKTEVQTGGFSKESILPKRNSDKLIAR
KKDWDPKKYGGFDSPTVAYSVLVVAKVEKGKSKKLKSVKELLGITIMERSSFEKNPIDFLEAKGYK
EVKKDLIIKLPKYSLFELENGRKRMLASAGELQKGNELALPSKYVNFLYLASHYEKLKGSPEDNEQK
QLFVEQHKHYLDEIIEQISEFSKRVILADANLDKVLSAYNKHRDKPIREQAENIIHLFTLTNLGAPAAF
KYFDTTIDRKRYTSTKEVLDATLIHQSITGLYETRIDLSQLGGDKRPAATKKAGQAKKKKGRADAL
DDFDLDMLGSDALDDFDLDMLGSDALDDFDLDMLGSDALDDFDLDMLSGGPKKKRKVGSQYLPD
TDDRHRIEEKRKRTYETFKSIMKKSPFSGPTDPRPPPRRIAVPSRSSASVPKPAPQPYPFTSSLSTINYD
EFPTMVFPSGQISQASALAPAPPQVLPQAPAPAPAPAMVSALAQAPAPVPVLAPGPPQAVAPPAPKP
TQAGEGTLSEALLQLQFDDEDLGALLGNSTDPAVFTDLASVDNSEFQQLLNQGIPVAPHTTEPMLM
EYPEAITRLVTGAQRPPDPAPAPLGAPGLPNGLLSGDEDFSSIADMDFSALLGSGSGSRDSREGMFLP
KPEAGSAISDVFEGREVCQPKRIRPFHPPGSPWANRPLPASLAPTPTGPVHEPVGSLTPAPVPQPLDPA
PAVTPEASHLLEDPDEETSQAVKALREMADTVIPQKEEAAICGQMDLSHPPPRGHLDELTTTLESMT
EDLNLDSPLTPELNEILDTFLNDECLLHAMHISTGLSIFDTSLFSGGKRPAATKKAGQAKKKKGSYPY
DVPDYA.

58-59. (canceled)

60. A vector comprising a nucleic acid molecule encoding the site-specific FOXP3 disrupting agent of claim 33; optionally, wherein the vector is a viral expression vector.

61. (canceled)

62. A cell comprising the site-specific FOXP3 disrupting agent of claim 33, optionally, wherein the cell is an immune cell.

63-64. (canceled)

65. The site-specific FOXP3 disrupting agent of claim 33, wherein the site-specific FOXP3 disrupting agent is present in a composition; optionally, wherein the composition comprises a pharmaceutical composition; optionally,
(a) wherein the pharmaceutical composition comprises a lipid formulation, wherein the lipid formulation comprises one or more cationic lipids, one or more non-cationic lipids, one or more cholesterol-based lipids, or one or more PEG-modified lipids, or combinations of any of the foregoing; and/or
(b) wherein the pharmaceutical composition comprises a lipid nanoparticle.

66-69. (canceled)

70. A method of modulating expression of forkhead box P3 (FOXP3) in a cell, the method comprising contacting the cell with the site-specific FOXP3 disrupting agent of claim 1, and an effector molecule, thereby modulating expression of FOXP3 in the cell.

71. The method of claim 70, wherein the modulation of expression is enhanced or reduced expression of FOXP3 in the cell;
(a) wherein the cell is a mammalian cell; optionally wherein the cell is a somatic cell or a primary cell;
(b) wherein the cell is an immune cell; optionally, wherein the immune cell is a naïve T cell or a regulatory T cell (Treg); and/or
(c) wherein the contacting is performed in vitro; in vivo; or ex vivo.

72-136. (canceled)

137. The method of claim 70, wherein the cell is within a subject; optionally, wherein the subject has a FOXP3-associated disease; optionally, wherein the FOXP3-associated disease is selected from the group consisting of IPEX syndrome (IPEX), type 1 diabetes, multiple sclerosis, systemic lupus erythematosus (SLE), and rheumatoid arthritis (RA).

138-139. (canceled)

140. A method for treating a subject having a FOXP3-associated disease, comprising administering to the subject a therapeutically effective amount of the site-specific FOXP3 disrupting agent of claim 1, and an effector molecule, thereby treating the subject.

141. The method of claim 140,
(a) wherein the FOXP3-associated disease is IPEX syndrome and the site-specific FOXP3 disrupting agent increases expression of FOXP3 in the subject;
(b) wherein the site-specific FOXP3 disrupting agent and the effector molecule are administered to the subject concurrently;
(c) wherein the site-specific FOXP3 disrupting agent and the effector molecule are administered to the subject sequentially;
(d) wherein the effector molecule is administered to the subject prior to administration of the site-specific FOXP3 disrupting agent; and/or
(e) wherein the site-specific FOXP3 disrupting agent is administered to the subject prior to administration of the effector molecule.

142-145. (canceled)


SEQUENCE LISTING (1090 sequences)

SEQ ID NO: 1 - DNA, 20 bp, synthetic construct
  misc_feature 1..20: note = Synthetic
  variation 1: note = a, c, t, g, unknown or other
  variation 3: note = a, c, t, g, unknown or other
  source 1..20: mol_type = other DNA
nbndccdshagrkgghrshv

SEQ ID NO: 2 - DNA, 20 bp, synthetic construct
  misc_feature 1..20: note = Synthetic
  variation 18: note = a, c, t, g, unknown or other
  variation 20: note = a, c, t, g, unknown or other
  source 1..20: mol_type = other DNA
vhsrhggkrgahsdccdnbn

SEQ ID NO: 3 - DNA, 2264 bp, Homo sapiens
  source 1..2264: mol_type = genomic DNA
agtttcccacaagccaggctgatccttttctgtcagtccacttcaccaagcctgcccttggacaaggacccgatgcccaaccccaggcctggcaagccctcggccccttccttggcccttggcccatccccaggagcctcgcccagctggagggctgcacccaaagcctcagacctgctgggggcccggggcccagggggaaccttccagggccgagatcttcgaggcggggcccatgcctcctcttcttccttgaaccccatgccaccatcgcagctgcagctgcccacactgcccctagtcatggtggcaccctccggggcacggctgggccccttgccccacttacaggcactcctccaggacaggccacatttcatgcaccagctctcaacggtggatgcccacgcccggacccctgtgctgcaggtgcaccccctggagagcccagccatgatcagcctcacaccacccaccaccgccactggggtcttctccctcaaggcccggcctggcctcccacctgggatcaacgtggccagcctggaatgggtgtccagggagccggcactgctctgcaccttcccaaatcccagtgcacccaggaaggacagcaccctttcggctgtgccccagagctcctacccactgctggcaaatggtgtctgcaagtggcccggatgtgagaaggtcttcgaagagccagaggacttcctcaagcactgccaggcggaccatcttctggatgagaagggcagggcacaatgtctcctccagagagagatggtacagtctctggagcagcagctggtgctggagaaggagaagctgagtgccatgcaggcccacctggctgggaaaatggcactgaccaaggcttcatctgtggcatcatccgacaagggctcctgctgcatcgtagctgctggcagccaaggccctgtcgtcccagcctggtctggcccccgggaggcccctgacagcctgtttgctgtccggaggcacctgtggggtagccatggaaacagcacattcccagagttcctccacaacatggactacttcaagttccacaacatgcgaccccctttcacctacgccacgctcatccgctgggccatcctggaggctccagagaagcagcggacactcaatgagatctaccactggttcacacgcatgtttgccttcttcagaaaccatcctgccacctggaagaacgccatccgccacaacctgagtctgcacaagtgctttgtgcgggtggagagcgagaagggggctgtgtggaccgtggatgagctggagttccgcaagaaacggagccagaggcccagcaggtgttccaaccctacacctggcccctgacctcaagatcaaggaaaggaggatggacgaacaggggccaaactggtgggaggcagaggtggtgggggcagggatgataggccctggatgtgcccacagggaccaagaagtgaggtttccactgtcttgcctgccagggcccctgttcccccgctggcagccaccccctcccccatcatatcctttgccccaaggctgctcagaggggccccggtcctggccccagcccccacctccgccccagacacaccccccagtcgagccctgcagccaaacagagccttcacaaccagccacacagagcctgcctcagctgctcgcacagattacttcagggctggaaaagtcacacagacacacaaaatgtcacaatcctgtccctcactcaacacaaaccccaaaacacagagagcctgcctcagtacactcaaacaacctcaaagctgcatcatcacacaatcacacacaagcacagccctgacaacccacacaccccaaggcacgcacccacagccagcctcagggcccacaggggcactgtcaacacaggggtgtgcccagaggcctacacagaagcagcgtcagtaccctcaggatctgaggtcccaacacgtgctcgctcacacacacggcctgttagaattcacctgtgtatctcacgcatatgcacacgcacagccccccagtgggtctcttgagtcccgtgcagacacacacagccacacacactgccttgccaaaaataccccgtgtctcccctgccactcacctcactcccattccctgagccctgatccatgcctcagcttagactgcagaggaactactcatttatttgggatccaaggcccccaacccacagtaccgtccccaataaactgcagccgagctcccca

SEQ ID NO: 4 - DNA, 2159 bp, Homo sapiens
  source 1..2159: mol_type = genomic DNA
agtttcccacaagccaggctgatccttttctgtcagtccacttcaccaagcctgcccttggacaaggacccgatgcccaaccccaggcctggcaagccctcggccccttccttggcccttggcccatccccaggagcctcgcccagctggagggctgcacccaaagcctcagacctgctgggggcccggggcccagggggaaccttccagggccgagatcttcgaggcggggcccatgcctcctcttcttccttgaaccccatgccaccatcgcagctgcagctctcaacggtggatgcccacgcccggacccctgtgctgcaggtgcaccccctggagagcccagccatgatcagcctcacaccacccaccaccgccactggggtcttctccctcaaggcccggcctggcctcccacctgggatcaacgtggccagcctggaatgggtgtccagggagccggcactgctctgcaccttcccaaatcccagtgcacccaggaaggacagcaccctttcggctgtgccccagagctcctacccactgctggcaaatggtgtctgcaagtggcccggatgtgagaaggtcttcgaagagccagaggacttcctcaagcactgccaggcggaccatcttctggatgagaagggcagggcacaatgtctcctccagagagagatggtacagtctctggagcagcagctggtgctggagaaggagaagctgagtgccatgcaggcccacctggctgggaaaatggcactgaccaaggcttcatctgtggcatcatccgacaagggctcctgctgcatcgtagctgctggcagccaaggccctgtcgtcccagcctggtctggcccccgggaggcccctgacagcctgtttgctgtccggaggcacctgtggggtagccatggaaacagcacattcccagagttcctccacaacatggactacttcaagttccacaacatgcgaccccctttcacctacgccacgctcatccgctgggccatcctggaggctccagagaagcagcggacactcaatgagatctaccactggttcacacgcatgtttgccttcttcagaaaccatcctgccacctggaagaacgccatccgccacaacctgagtctgcacaagtgctttgtgcgggtggagagcgagaagggggctgtgtggaccgtggatgagctggagttccgcaagaaacggagccagaggcccagcaggtgttccaaccctacacctggcccctgacctcaagatcaaggaaaggaggatggacgaacaggggccaaactggtgggaggcagaggtggtgggggcagggatgataggccctggatgtgcccacagggaccaagaagtgaggtttccactgtcttgcctgccagggcccctgttcccccgctggcagccaccccctcccccatcatatcctttgccccaaggctgctcagaggggccccggtcctggccccagcccccacctccgccccagacacaccccccagtcgagccctgcagccaaacagagccttcacaaccagccacacagagcctgcctcagctgctcgcacagattacttcagggctggaaaagtcacacagacacacaaaatgtcacaatcctgtccctcactcaacacaaaccccaaaacacagagagcctgcctcagtacactcaaacaacctcaaagctgcatcatcacacaatcacacacaagcacagccctgacaacccacacaccccaaggcacgcacccacagccagcctcagggcccacaggggcactgtcaacacaggggtgtgcccagaggcctacacagaagcagcgtcagtaccctcaggatctgaggtcccaacacgtgctcgctcacacacacggcctgttagaattcacctgtgtatctcacgcatatgcacacgcacagccccccagtgggtctcttgagtcccgtgcagacacacacagccacacacactgccttgccaaaaataccccgtgtctcccctgccactcacctcactcccattccctgagccctgatccatgcctcagcttagactgcagaggaactactcatttatttgggatccaaggcccccaacccacagtaccgtccccaataaactgcagccgagctcccca

SEQ ID NO: 5 - DNA, 14497 bp, Homo sapiens
  source 1..14497: mol_type = genomic DNA
aaaatttcaaaatttccgtttaagtctcataatcaagaaaaggagaaacacagagagagagaaaaaaaaaactatgagaacccctccccaccccgtgattatcagcgcacacactcatcgaaaaaaatttggattattagaagagagaggtctgcggcttccacaccgtacagcgtggtttttcttctcggtataaaagcaaagttgtttttgatacgtgacagtttcccacaagccaggctgatccttttctgtcagtccacttcaccaaggtgagtgtccctgctctcccctaccagatgtgggccccattggaggagatggcagggaggtaggcacggcgggggggtcaggggccctctggtacagtgggatgtacccagctaccgtgattccagccaggtaaggtctttaaaaaaataatagaataaatggcagaagacttaaaggtgaagtacttaatgcatgggcaggggcaaaaataaatgaattcatcaattgatacgtagatggataggatttttaaaatatgtcagctgccagtctttatggatttcagagctttgaacaacttgaaatgctgtccaggtgcaactgaaaatttcaactgcatctctctcatctctttgtctcttagctcttgatgcctctcatccttctctgtctctctgctcaccctgtcttgctcttttacatgtttctctgcctctctttctcccgccatctgtctgtcgctacactcagactctctttccccgcatctctcctggcctctgaccatctcccctatctctgtctcctccatcttgaccacactccatcgatctcctttcctccatctctcccgctctcttttcttgccttcccccacctgccttcccagccagcatccagcctagagctttgtacacagctggcgtttaataatagtaatgtcgccgaggacctttacattttatgagagtgagcatcagttctgttcaccctagtagcaggaggattgcaaggtgccggggcaagctgagcacttaactgaactttgcttgtatcgctttagaggagacccttggggcgggggggcagggagcacttcacaccattgtcaaccgtagtttagctgtgtggacctaccccaggactgcatgtgtgtgaccagagtccatattcaggaagcaaacaggttgttcagggctggggtgagctcgattctgcaggcttagcacagcgtttggcacaaagtaagtgctcacgaagtgttagctattagtatttctgtaaccaaattagaatcatgctatatattggttttgttcctgctttattcaattaacagtagtgtttttgaagcttactaaaaattgtttgaacacatgatctttcatgatggacaagactgtcctcacatcagcatgaactttgattgatttgaccaagtgccgattgttggacattccggttgtttctcgtttttttctataaacatccctgactgcttccttggattcctgaaaagggagttgctggggcaaagggggcgtgcatttttaaagtttatgaccctctctgctaattctccttccaaaaggttgtcctattttatgctccctctacttaagctaaccctaggtgagtattattatgttttaattatttcccaacatgacaggtaagaaaaacacattatcacattgttgaaatttgcatcttgtgattacttttactattcattggccattcatgcttttttttttttttttttggttgtctggtcatgtccttactcctaatagggtgttcatcttatttttcccttattgatttgccacagctctttatttggaatggttattaacctttcatcagccattacatatatagcaaatattttccccaattcatcatttgctttttttctatttgtttatggtgttttttgtgatgcttatgatggttttatacagtcaaataggttagtctttttttctgtggcttctgtctctggttttgtgcttagaaagtcctttcctacttgaaaatgagataaatgttcacctatgttggcttctagtctcttttatggcttcattttttccatttactatagaggttaagagtgtgggtactggagccagactgtctgggacaaacccagcgtcaccccaagccctatgtgtgatttttagccaggcacttaacctctccatacctccatttcctcatatgtactgcaatggttataatagtaccttcctcaggagtctttgtttagattaaaatttttaaccacagtaaatacttagcacaaggcctgacacacaataaacccaagatcagcattaggtgttaaaacttatatcttgaatggatctgggcattttagggtatatgatgatggtgacatttcaaactgggcagggaggggttgttgggataatggactgactattcactcaataactttatcttctccctaattatctcagacacatttgttaaaccatactccatggtcctccagtttgaaatgccacattcatcacaaattcagttctctccatatgtgggtccatgtccaagctttctattctgttctcttccccagtatctgttcttataccagggccacactgttttgctgattgttgctttgcaatacaattcaatacccagccatgggtgtccctggcaccttgcccttctttattcttgattatttcaggcagcatcagttgaaccagccagagaccagcaaatgttcttatttgaggcaatcctcctctcgcacacatgcactctggctctccatgcatgtgtccatttctctcaatgtctctcactatctcttgttctctcttgctcgctctttgtgtgtgtctctttgtctctatgtatccctatctctcagccagtcccctggatagaggggcaattgtcaacttgaagccctgcagacacctaatgacttaaccagacagcgtagaagggccctggcccttgtctactccacgcctctccgtgctcagtgtagaagggcaaattgaagaccagagatctcagggcctcaccgaaatgcagcggcagagttgaaatccaagcctagatctcaggactctaggtgggaccctggttctggctctctccccaactgcaggcctcagtttacccctcagcacccagaagggggaaggggaacctgggctaccattcccccttctgccttctcacacgttggaccccaacttcccacaggttggacgatccacgatcacagtgtggggcccagcctcacaagagctgggctaggtgaggccccggactccataggtcaggaggcctagttggccagagcgtggtgatgatggaggcatgtcagtcagtcaggctgtgtgtccccagagctggtgctggtccccgaaaaccttgattgtggggcccctctagagagtctgatgatgggctctgtattggcgaaggctgaggcttttccagctccccccatgaggcccagaccaaaggcacaccagcctcaacctcctcctccccctgttgccatctctggcggagtggccatgtatttgggaacgttgttccagagtggacagggagactgaggccctagggaggctggctctgtttccaggcctggtaggcaagaggccctatgaagcagcaagctgcctgactttcagatggttccaaggagtttggacaccagggacactggcctacacatactgagactttgggaccgtagaccccacagtctgtggttttgagattctaggatcctttaaatctaagaaatgctgttctatgattctgaggtcctggtgttatactatttgaagaccccaggggtcccagtatctgtggagcctgcctggcactctcagagcttcaaacctgggtcctctccacaacccaagaagggccaggtcttcagagctaggggcttgtcatagtggccagatggacatcacctaccacatccaccagcacccatgtcaccccacctgggccaagcctgctgcaggacagggcagccagttctcggaacgaaacctgtggggtggggtatctgccctcttctcttcctccgtggtgtcgatgaagcccggcgcatccggccgccatgacgtcaatggcggaaaaatctgggcaagtcgggggctgtgacaacagggcccagatgcagaccccgatatgaaaacataatctgtgtcccagaaacatcccccattcagcttctgagaaacccagtcagaaagggacgtcccaacagacagtgcaggaagccggctgcccagcccggccctctaggtcctctacccccagacagatcatctccatgtccctgtctgagaatgtatctatgctttgctgagtcaggccatcccacatgtgtttggggagaattcttagctctggccaagtgtccaggcagcttcagaagtgaccacaggccagccacatgggccaggccagagtggtggaaaacatccatttgcaccgaaatcggtattagtttgttctggctgctataacaaagtaccacaactgagtggcttcagcaacagaaattgatcatctcacagttctggaggccagagtccaagatcaaggtgttgccagggttggttccttctgggggccataagggagaagctgctccaggcctctcccccagcttctggtggttgctggcaatctttgttatttcttggcttgaaggagcatcaccccatctctggcttcattcattcactcaccttctcatctcaccagtgctctcccaggcccttgttcggagcctccacagacccacactcctgtttctcacgcagtaatgttctaagcccccaggagactaagaacttaatacctggattctcacactcatctccctcagcaccacagctctgacaaccacccccaggaggtgacagcaaagaaaggaaggagcaactgctcccccagaacccttctatactcccccgggctgctctcagcagctgtgtgcccaactcagtgctgtgaggtggaacggggagatggagacacacacacacacacaccagagagtttataaaatgtgcacacggaaacacttcctaaagaaagaagtggagttctcaagtcactatgggactacggaactcggcatctgagcatccccttctgtataatggggttggctgggcagtcccttccagctctggcttcctctcagaccaggattataggatcctgaaaagccagtgcttccctcagggagtggaaggttctggggctcagcagggggcccggagccattgtggagggctttcaaggtgaggacaatagaagagcaggtcttgcgtggcttggacagggaaagaggagaaggagtgggcatttgaggcagggggtgtagcatgagccaggcttgagggccagaaattggggtgaactctgatgggggctgggtagagaagcttctacaggccccagctcaagagaccccatctctcctcctctctgtcacttgccatgctggatccgtgcatgatcacactcctggactcgcctccttgccctgagatccagacccccgtattcagctgccccctcagctcctccactcacatatttaatgccagactcttcatgtctatctacacctgcacttttgcacccaatccaactccccgccatgtcccccatctcaggtaatgtcagctcggtccttccagctgctcaagctaaaacccatgtcactttgactctccctcttgcccactacatccaagctgctagcactgctcctgatccagcttcagattaagtctcagaatctacccacttctcgccttctccactgccaccagcccattctgtgccagcatcatcacttgccaggactgttacaatagcctcctcactagccccactcacagcagccagatgaatcttttgagtccatgcctagtcactggggcaaaataggactccgaggagaaagtccgagaccagctccggcaagatgagcaaacacagcctgtgcagggtgcagggagggctagaggcctgaggcttgaaacagctctcaagtggagggggaaacaaccattgccctcatagaggacacatccacaccagggctgtgctagcgtgggcaggcaagccaggtgctggacctctgcacgtggggcatgtgtgggtatgtacatgtacctgtgttcttggtgtgtgtgtgtgtgtgtgtgtgtgtgtgtgtctagagctggggtgcaactatggggcccctcgggacatgtcccagccaatgcctgctttgaccagaggagtgtccacgtggctcaggtggtcgagtatctcataccgccctagcacacgtgtgactcctttcccctattgtctacgcagcctgcccttggacaaggacccgatgcccaaccccaggcctggcaagccctcggccccttccttggcccttggcccatccccaggagcctcgcccagctggagggctgcacccaaagcctcagacctgctgggggcccggggcccagggggaaccttccagggccgagatcttcgaggcggggcccatgcctcctcttcttccttgaaccccatgccaccatcgcagctgcaggtgaggccctgggcccaggatggggcaggcagggtggggtacctggacctacaggtgccgacctttactgtggcactgggcgggaggggggctggctggggcacaggaagtggtttctgggtcccaggcaagtctgtgacttatgcagatgttgcagggccaagaaaatccccacctgccaggcctcagagattggaggctctccccgacctcccaatccctgtctcaggagaggaggaggccgtattgtagtcccatgagcatagctatgtgtccccatccccatgtgacaagagaagaggactggggccaagtaggtgaggtgacagggctgaggccagctctgcaacttattagctgtttgatctttaaaaagttactcgatctccatgagcctcagtttccatacgtgtaaaagggggatgatcatagcatctaccatgtgggcttgcagtgcagagtatttgaattagacacagaacagtgaggatcaggatggcctctcacccacctgcctttctgcccagctgcccacactgcccctagtcatggtggcaccctccggggcacggctgggccccttgccccacttacaggcactcctccaggacaggccacatttcatgcaccaggtatggacggtgaatgggcagggaggagggagcaggtgggagaactgtggggaggggccccgagtcaggctgaaccacagcccacatgtgccccccagctctcaacggtggatgcccacgcccggacccctgtgctgcaggtgcaccccctggagagcccagccatgatcagcctcacaccacccaccaccgccactggggtcttctccctcaaggcccggcctggcctcccacctggtaacacctcagcccgtaccccatggcttcacagaacccccaagtccccagatccttggctgtgagcagtgtaggctattctgaattgcagtactctgggggtcaaaggtgtcaggtctcagaggcttggaaactccaccctccaaaaaacgtcaggtgcagaaccttaaagatgcagaatgtcaaaatcacaaaaccacagagctttacaaagctagtcaaaatgtcagcacctgcgaatggccgtctttaagcttctctgccagaagcctgggactttggggacagcagagccccctgggagtcagggttttcgaggctcaggagggtgggaagctcaaaatgagaggccttgtgggccaagctccagagcccagcccacagcctccataggtgccctgtccccacccacagggatcaacgtggccagcctggaatgggtgtccagggagccggcactgctctgcaccttcccaaatcccagtgcacccaggaaggacaggtcagtggacagggctgggaaggatcctcgccctcctatcccctcccctgacaccctctgtccccccagcaccctttcggctgtgccccagagctcctacccactgctggcaaatggtgtctgcaagtggcccggatgtgagaaggtcttcgaagagccagaggacttcctcaagtgagtggcccaggcctgtgccagtctaccggccctggcttgtggggagaggtctagggtgcagatctcagctcaggcttcatcccaataagtagtaagatgcaagagctaaactctgagactgtgggttcaaatcccagctccatccctgactggctgtgtgaccttggacaaattacttagcttctctcgacctcagttttctcgtatgtataataagaaacgtttgtgctgttatttacccccattttacagacaaggagactgaggccaaagaggttaagtgtcttgatcaaaatctcccagttcctgagtgacagagctgggatttgaacccatgtagccaggctctagaatctgcactcctgctttgccctgctactcctcatgccaacatgccagccttattccactgttcccaaagttctagctcctctagatggctgctgctgcctcccctcagtccccccatatgtctctctctctctctctctctctctctctctctctctctctctctctctctctctttctctctctctcacacacacacacacacaaatacacaccacactacacacaacccttagcagcaaccccacatgtcctgtcctccttgcagccaggcctttgcacaggccgtccctccacccagaatgctttgcccactctcacctccctggcccattctcaactcaccacacttccaacattatctccagcactctggccaggctcaagtggtgagttcaggcctgacatgcagtaggtgctgaggggcatgtgttaagggaacgaggggtgtgagagggagactgaggtagagaggggaggggagctggggctcagaggagagaactcccagagggtctgggccctccccattcagagcattgagccagaccaggcctgtcgtggtcacctgcatggaatcttctccctacttaggcactgccaggcggaccatcttctggatgagaagggcagggcacaatgtctcctccagagagagatggtacagtctctggagcagcaggtaatgccagggcggtggagggtaagggatagggatagtgcgcaaaaccttctgtccaccatgtgccagaaaccaagttcacctgggacgagggctggtataaaggaaggaagaggagcgggcactcccagggaagaccgtagcctgggcaaagatgtggcagagaagggccaggctgaggcctcatgtttgtgccatttcacagctggtgctggagaaggagaagctgagtgccatgcaggcccacctggctgggaaaatggcactgaccaaggcttcatctgtggtgagcgaccccaggactggtggtggcagactcagactgctggggggcagggaggaggtctgcacggtgcagggaacctaacctcacattcaggtcctgagagctaggggcccctgtccccagctccaaacatgcccagacctggaaatctgtccccttctacttacaaaccctctgaccccggctgggcgtggtggctcacgcctgtaatcccagcactttgggaggccgaggcgggtggatcacgaggtcaggagttcaagaccagcctcgccaagatggtgaaaccctgtgtctactaaaagtacaaaaaattagccaggcgtggtggcaggtgtctgtagtcccaactacttgggaggctgaggcagagaattgcttgaacctgggaggcggagtttgcagtgagctgagatcataccactgcactccagcctaggcgacagatcaagactccgtctcaagaaaaaaaaaaaaaaaaaaccctctgactctaagatccccaaacactgtgatcctgagttgttaaagcaaatgcaaatagccagacttgccagatgcaggctgtgtgccagcaggaccagctatgtaacctgcagggccccttgttcagatttcaagatggcgaccacagagcattaaacaaagcacagggtgccacataaccacataggtcacacgcccatgaagccagccctggaggccaggcactgtttctgagcgctttgctggtggtaatttatttctcatgatgcgaatgtacagatgaggaatattgaggccaggggggtttaggtgactttcccaaggtcacagttgggtggtaaagagccctattcaaccccagttcatggtcccagcatcagtggccacatacgacatccgcacctgtgctctaataaatacggctcatgctgttttgtgggattccacctcagactggaatttagaagagggcgtccttgctttgaaaaacactgatttaaaaataaagtggagccaggcgcagtggctcatgcctgtaatcccagcactttgggaggctgaagcgggtggatcacatgaggtcaggagttcgagaccagcctggctaacatggtgaaaccccatctctaccaaaaatacaaaaattagctgggcgtagtgacgggcacctgtagtcccagctactcgggaggctgaggcagaagacttgcttgaatctgggaggtgggggttgcagtgagccgagattgcaccactgcactccagcctgggcgacagagcaagactcagtatcaaaaaaaataaaaaacataaaatagaaagtaaaaagtgggaagtttaagcctctgggtcaccagcctctccccctcacccaggcatcatccgacaagggctcctgctgcatcgtagctgctggcagccaaggccctgtcgtcccagcctggtctggcccccgggaggcccctgacagcctgtttgctgtccggaggcacctgtggggtagccatggaaacagcacattcccaggtaagaatggtccttgcactacacggtgcccccaagctcctaatcctgacaggctctgggtggagggtgcaaaggagctccatgctgccccttcccaccaccaccaccactgcagctgccgccactcagcctttgggaaaatgcatccgctcacaaaagcttcctttcgggatgtccgtggcctgaaagccccccatatggtctcgagtgtcgggcccctagccctgactcccttggggattggggccatgcctcacccactctggactccagctactatattcggccatcagaagggagggaccctgctaagtaattccaggagcacctcctttcctcccctgaccaaggaaaatcggggtggattcgcccgagctcacctatccactgctctccaccaggcctggcctgtgggcttagcagggatcagagaccttgactgtcatcctggctctgccatttaacctcttgcatcctttggtgtgcaagttactccgcttcttttcaacctcggggagaactattttggcagaagtggtgcaaagaataaatgatacaacttatgtcaggtgctcagcaaacagtacctgtgcccgtggacacgggtgttgacggtgagatctcaggcctgtagactcaccttgtagggggaggggacagggagctagctaggaggtcctgcatggggcttgattcatccccaccctctgacagagttcctccacaacatggactacttcaagttccacaacatgcgaccccctttcacctacgccacgctcatccgctgggtaagcagggcagctcggccccaaggaggaggaagacaaagatggggtgggggacctgcctcccaaactcctgtctccctctgagtgccctcagagtgggttcctccattcccaagccccagccccaaggatccccaagccgtgcctcaaatgtgacccctcatgctggcttcaccccaaacctgtccgcaaatccaaacctaaaccaccatccaggccagagcatgccaaattctgaccctaaacctacccctttccagatgtccacctcagccccatacctaaccctctcctggacccataaaatagcctaaagctaaccccatctctgcaccttgccctaaacgtaccccagctcttactctaactccttccccagcctttatgccaacccaaccccatctccttccctggccccaacgtaccccagtatgtcaatacacccccaactgggcaccattcccaacctttccttgtaacacccatttgatccttaacttcatcaccccatcccaactccttcatctcagcctccccaatgccttctcagaaccttcatcctagcgccacacctaaccccaaacctgaacctcacccctacatgataccagatattccccgactgtctctgaactgaaaccctgacctagccccatcctgactgatagcctcactgcaatcatcatcgctgactctgtaatcccattcctgaatccaaactgatcaaattccctgaccctcagcctcactccacaccgaaccccacaactaacctcatccttgccctgagcctaaccacctaaccatgtccctgacacgtaagatactcgataattcaaaccatctctggcttctgacctaaagccaagtcatcccatccctcacccctagaccccccacctgagtccaccccagcctgactcttaacccaccccttccctgacccttaagttggcctggccctttaccccttccaaatccttgaccccacttctgacccttcaccttccccaaccctgaaatgtcacctccacagagaaccccaactccaactccatcccagacctttcacctcacttcagccctagccctgaacaagaccccactcccaacctcagtcctgatcccttacctaatcccagaacaaccatacccacacccatctaaccctgcccaacccgacacttcaccccttttctaaccccatctttgaccccatgcttcaccccacatctagtcctgtccctgattacctgcccctacaattccgcccccatgtcagatggctcggggtaggtcatagcccctctaaaccccaagtttggggaatgtgccccttaccccaccccccaacttccaggccatcctggaggctccagagaagcagcggacactcaatgagatctaccactggttcacacgcatgtttgccttcttcagaaaccatcctgccacctggaaggtgagctcctctgaggtggcggtgactgggatggcctcaagtgccatcgcagctcaaagtgggcaggcctgggtctgggctcataggcacattggggaggaacgggatgtgggttgttggtggtggctgctggcctcagaggttgacgcccacctgctccctgtccccggccttccacagaacgccatccgccacaacctgagtctgcacaagtgctttgtgcgggtggagagcgagaagggggctgtgtggaccgtggatgagctggagttccgcaagaaacggagccagaggcccagcaggtgttccaaccctacacctggcccctgacctcaagatcaaggaaaggaggatggacgaacaggggccaaactggtgggaggcagaggtggtgggggcagggatgataggccctggatgtgcccacagggaccaagaagtgaggtttccactgtcttgcctgccagggcccctgttcccccgctggcagccaccccctcccccatcatatcctttgccccaaggctgctcagaggggccccggtcctggccccagcccccacctccgccccagacacaccccccagtcgagccctgcagccaaacagagccttcacaaccagccacacagagcctgcctcagctgctcgcacagattacttcagggctggaaaagtcacacagacacacaaaatgtcacaatcctgtccctcactcaacacaaaccccaaaacacagagagcctgcctcagtacactcaaacaacctcaaagctgcatcatcacacaatcacacacaagcacagccctgacaacccacacaccccaaggcacgcacccacagccagcctcagggcccacaggggcactgtcaacacaggggtgtgcccagaggcctacacagaagcagcgtcagtaccctcaggatctgaggtcccaacacgtgctcgctcacacacacggcctgttagaattcacctgtgtatctcacgcatatgcacacgcacagccccccagtgggtctcttgagtcccgtgcagacacacacagccacacacactgccttgccaaaaataccccgtgtctcccctgccactcacctcactcccattccctgagccctgatccatgcctcagcttagactgcagaggaactactcatttatttgggatccaaggcccccaacccacagtaccgtccccaataaactgcagccgagctccccaca

SEQ ID NO: 6 - RNA, 4487 nt, synthetic construct
  misc_feature 1..4487: note = source = /note="Description of Artificial Sequence: Syntheticpolynucleotide"
  source 1..4487: mol_type = other RNA
aaggaaataagagagaaaagaagagtaagaagaaatataagagccaccatggcccccaagaagaagcggaaggtgggcatccacggcgtgcccgccgccgacaagaagtacagcatcggcctggccatcggcaccaacagcgtgggctgggccgtgatcaccgacgagtacaaggtgcccagcaagaagttcaaggtgctgggcaacaccgaccggcacagcatcaagaagaacctgatcggcgccctgctgttcgacagcggcgagaccgccgaggccacccggctgaagcggaccgcccggcggcggtacacccggcggaagaaccggatctgctacctgcaggagatcttcagcaacgagatggccaaggtggacgacagcttcttccaccggctggaggagagcttcctggtggaggaggacaagaagcacgagcggcaccccatcttcggcaacatcgtggacgaggtggcctaccacgagaagtaccccaccatctaccacctgcggaagaagctggtggacagcaccgacaaggccgacctgcggctgatctacctggccctggcccacatgatcaagttccggggccacttcctgatcgagggcgacctgaaccccgacaacagcgacgtggacaagctgttcatccagctggtgcagacctacaaccagctgttcgaggagaaccccatcaacgccagcggcgtggacgccaaggccatcctgagcgcccggctgagcaagagccggcggctggagaacctgatcgcccagctgcccggcgagaagaagaacggcctgttcggcaacctgatcgccctgagcctgggcctgacccccaacttcaagagcaacttcgacctggccgaggacgccaagctgcagctgagcaaggacacctacgacgacgacctggacaacctgctggcccagatcggcgaccagtacgccgacctgttcctggccgccaagaacctgagcgacgccatcctgctgagcgacatcctgcgggtgaacaccgagatcaccaaggcccccctgagcgccagcatgatcaagcggtacgacgagcaccaccaggacctgaccctgctgaaggccctggtgcggcagcagctgcccgagaagtacaaggagatcttcttcgaccagagcaagaacggctacgccggctacatcgacggcggcgccagccaggaggagttctacaagttcatcaagcccatcctggagaagatggacggcaccgaggagctgctggtgaagctgaaccgggaggacctgctgcggaagcagcggaccttcgacaacggcagcatcccccaccagatccacctgggcgagctgcacgccatcctgcggcggcaggaggacttctaccccttcctgaaggacaaccgggagaagatcgagaagatcctgaccttccggatcccctactacgtgggccccctggcccggggcaacagccggttcgcctggatgacccggaaatccgaggagaccatcaccccctggaacttcgaggaggtggtggacaagggcgccagcgcccagagcttcatcgagcggatgaccaacttcgacaagaacctgcccaacgagaaggtgctgcccaagcacagcctgctgtacgagtacttcaccgtgtacaacgagctgaccaaggtgaagtacgtgaccgagggcatgcggaagcccgccttcctgagcggcgagcagaagaaggccatcgtggacctgctgttcaagaccaaccggaaggtgaccgtgaagcagctgaaggaggactacttcaagaagatcgagtgcttcgacagcgtggagatcagcggcgtggaggaccggttcaacgccagcctgggcacctaccacgacctgctgaagatcatcaaggacaaggacttcctggacaacgaggagaacgaggacatcctggaggacatcgtgctgaccctgaccctgttcgaggaccgggagatgatcgaggagcggctgaaaacctacgcccacctgttcgacgacaaggtgatgaagcagctgaagcggcggcggtacaccggctggggccggctgagccggaagctgatcaacggcatccgggacaagcagagcggcaagaccatcctggacttcctgaaatccgacggcttcgccaaccggaacttcatgcagctgatccacgacgacagcctgaccttcaaggaggacatccagaaggcccaggtgagcggccagggcgacagcctgcacgagcacatcgccaacctggccggcagccccgccatcaagaagggcatcctgcagaccgtgaaggtggtggacgagctggtgaaggtgatgggccggcacaagcccgagaacatcgtgatcgagatggcccgggagaaccagaccacccagaagggccagaagaacagccgggagcggatgaagcggatcgaggagggcatcaaggagctgggcagccagatcctgaaggagcaccccgtggagaacacccagctgcagaacgagaagctgtacctgtactacctgcagaacggccgggacatgtacgtggaccaggagctggacatcaaccggctgagcgactacgacgtggccgccatcgtgccccagagcttcctgaaggacgacagcatcgacaacaaggtgctgacccggagcgacaaggcccggggcaagagcgacaacgtgcccagcgaggaggtggtgaagaagatgaagaactactggcggcagctgctgaacgccaagctgatcacccagcggaagttcgacaacctgaccaaggccgagcggggcggcctgagcgagctggacaaggccggcttcatcaagcggcagctggtggagacccggcagatcaccaagcacgtggcccagatcctggacagccggatgaacaccaagtacgacgagaacgacaagctgatccgggaggtgaaggtgatcaccctgaaatccaagctggtgagcgacttccggaaggacttccagttctacaaggtgcgggagatcaacaactaccaccacgcccacgacgcctacctgaacgccgtggtgggcaccgccctgatcaagaagtaccccaagctggagagcgagttcgtgtacggcgactacaaggtgtacgacgtgcggaagatgatcgccaagagcgagcaggagatcggcaaggccaccgccaagtacttcttctacagcaacatcatgaacttcttcaagaccgagatcaccctggccaacggcgagatccggaagcggcccctgatcgagaccaacggcgagaccggcgagatcgtgtgggacaagggccgggacttcgccaccgtgcggaaggtgctgagcatgccccaggtgaacatcgtgaagaaaaccgaggtgcagaccggcggcttcagcaaggagagcatcctgcccaagcggaacagcgacaagctgatcgcccggaagaaggactgggaccccaagaagtacggcggcttcgacagccccaccgtggcctacagcgtgctggtggtggccaaggtggagaagggcaagagcaagaagctgaaatccgtgaaggagctgctgggcatcaccatcatggagcggagcagcttcgagaagaaccccatcgacttcctggaggccaagggctacaaggaggtgaagaaggacctgatcatcaagctgcccaagtacagcctgttcgagctggagaacggccggaagcggatgctggccagcgccggcgagctgcagaagggcaacgagctggccctgcccagcaagtacgtgaacttcctgtacctggccagccactacgagaagctgaagggcagccccgaggacaacgagcagaagcagctgttcgtggagcagcacaagcactacctggacgagatcatcgagcagatcagcgagttcagcaagcgggtgatcctggccgacgccaacctggacaaggtgctgagcgcctacaacaagcaccgggacaagcccatccgggagcaggccgagaacatcatccacctgttcaccctgaccaacctgggcgcccccgccgccttcaagtacttcgacaccaccatcgaccggaagcggtacaccagcaccaaggaggtgctggacgccaccctgatccaccagagcatcaccggcctgtacgagacccggatcgacctgagccagctgggcggcgacagcggcggcaagcggcccgccgccaccaagaaggccggccaggccaagaagaagaagggcagctacccctacgacgtgcccgactacgcctgagcggccgcttaattaagctgccttctgcggggcttgccttctggccatgcccttcttctctcccttgcacctgtacctcttggtctttgaataaagcctgagtaggaagtctagaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 7 - RNA, 6394 nt, synthetic construct
  misc_feature 1..6394: note = source = /note="Description of Artificial Sequence: Syntheticpolynucleotide"
  source 1..6394: mol_type = other RNA
aggaaataagagagaaaagaagagtaagaagaaatataagagccaccatggcccccaagaagaagcggaaggtgggcatccacggcgtgcccgccgccgacaagaagtacagcatcggcctggccatcggcaccaacagcgtgggctgggccgtgatcaccgacgagtacaaggtgcccagcaagaagttcaaggtgctgggcaacaccgaccggcacagcatcaagaagaacctgatcggcgccctgctgttcgacagcggcgagaccgccgaggccacccggctgaagcggaccgcccggcggcggtacacccggcggaagaaccggatctgctacctgcaggagatcttcagcaacgagatggccaaggtggacgacagcttcttccaccggctggaggagagcttcctggtggaggaggacaagaagcacgagcggcaccccatcttcggcaacatcgtggacgaggtggcctaccacgagaagtaccccaccatctaccacctgcggaagaagctggtggacagcaccgacaaggccgacctgcggctgatctacctggccctggcccacatgatcaagttccggggccacttcctgatcgagggcgacctgaaccccgacaacagcgacgtggacaagctgttcatccagctggtgcagacctacaaccagctgttcgaggagaaccccatcaacgccagcggcgtggacgccaaggccatcctgagcgcccggctgagcaagagccggcggctggagaacctgatcgcccagctgcccggcgagaagaagaacggcctgttcggcaacctgatcgccctgagcctgggcctgacccccaacttcaagagcaacttcgacctggccgaggacgccaagctgcagctgagcaaggacacctacgacgacgacctggacaacctgctggcccagatcggcgaccagtacgccgacctgttcctggccgccaagaacctgagcgacgccatcctgctgagcgacatcctgcgggtgaacaccgagatcaccaaggcccccctgagcgccagcatgatcaagcggtacgacgagcaccaccaggacctgaccctgctgaaggccctggtgcggcagcagctgcccgagaagtacaaggagatcttcttcgaccagagcaagaacggctacgccggctacatcgacggcggcgccagccaggaggagttctacaagttcatcaagcccatcctggagaagatggacggcaccgaggagctgctggtgaagctgaaccgggaggacctgctgcggaagcagcggaccttcgacaacggcagcatcccccaccagatccacctgggcgagctgcacgccatcctgcggcggcaggaggacttctaccccttcctgaaggacaaccgggagaagatcgagaagatcctgaccttccggatcccctactacgtgggccccctggcccggggcaacagccggttcgcctggatgacccggaaatccgaggagaccatcaccccctggaacttcgaggaggtggtggacaagggcgccagcgcccagagcttcatcgagcggatgaccaacttcgacaagaacctgcccaacgagaaggtgctgcccaagcacagcctgctgtacgagtacttcaccgtgtacaacgagctgaccaaggtgaagtacgtgaccgagggcatgcggaagcccgccttcctgagcggcgagcagaagaaggccatcgtggacctgctgttcaagaccaaccggaaggtgaccgtgaagcagctgaaggaggactacttcaagaagatcgagtgcttcgacagcgtggagatcagcggcgtggaggaccggttcaacgccagcctgggcacctaccacgacctgctgaagatcatcaaggacaaggacttcctggacaacgaggagaacgaggacatcctggaggacatcgtgctgaccctgaccctgttcgaggaccgggagatgatcgaggagcggctgaaaacctacgcccacctgttcgacgacaaggtgatgaagcagctgaagcggcggcggtacaccggctggggccggctgagccggaagctgatcaacggcatccgggacaagcagagcggcaagaccatcctggacttcctgaaatccgacggcttcgccaaccggaacttcatgcagctgatccacgacgacagcctgaccttcaaggaggacatccagaaggcccaggtgagcggccagggcgacagcctgcacgagcacatcgccaacctggccggcagccccgccatcaagaagggcatcctgcagaccgtgaaggtggtggacgagctggtgaaggtgatgggccggcacaagcccgagaacatcgtgatcgagatggcccgggagaaccagaccacccagaagggccagaagaacagccgggagcggatgaagcggatcgaggagggcatcaaggagctgggcagccagatcctgaaggagcaccccgtggagaacacccagctgcagaacgagaagctgtacctgtactacctgcagaacggccgggacatgtacgtggaccaggagctggacatcaaccggctgagcgactacgacgtggccgccatcgtgccccagagcttcctgaaggacgacagcatcgacaacaaggtgctgacccggagcgacaaggcccggggcaagagcgacaacgtgcccagcgaggaggtggtgaagaagatgaagaactactggcggcagctgctgaacgccaagctgatcacccagcggaagttcgacaacctgaccaaggccgagcggggcggcctgagcgagctggacaaggccggcttcatcaagcggcagctggtggagacccggcagatcaccaagcacgtggcccagatcctggacagccggatgaacaccaagtacgacgagaacgacaagctgatccgggaggtgaaggtgatcaccctgaaatccaagctggtgagcgacttccggaaggacttccagttctacaaggtgcgggagatcaacaactaccaccacgcccacgacgcctacctgaacgccgtggtgggcaccgccctgatcaagaagtaccccaagctggagagcgagttcgtgtacggcgactacaaggtgtacgacgtgcggaagatgatcgccaagagcgagcaggagatcggcaaggccaccgccaagtacttcttctacagcaacatcatgaacttcttcaagaccgagatcaccctggccaacggcgagatccggaagcggcccctgatcgagaccaacggcgagaccggcgagatcgtgtgggacaagggccgggacttcgccaccgtgcggaaggtgctgagcatgccccaggtgaacatcgtgaagaaaaccgaggtgcagaccggcggcttcagcaaggagagcatcctgcccaagcggaacagcgacaagctgatcgcccggaagaaggactgggaccccaagaagtacggcggcttcgacagccccaccgtggcctacagcgtgctggtggtggccaaggtggagaagggcaagagcaagaagctgaaatccgtgaaggagctgctgggcatcaccatcatggagcggagcagcttcgagaagaaccccatcgacttcctggaggccaagggctacaaggaggtgaagaaggacctgatcatcaagctgcccaagtacagcctgttcgagctggagaacggccggaagcggatgctggccagcgccggcgagctgcagaagggcaacgagctggccctgcccagcaagtacgtgaacttcctgtacctggccagccactacgagaagctgaagggcagccccgaggacaacgagcagaagcagctgttcgtggagcagcacaagcactacctggacgagatcatcgagcagatcagcgagttcagcaagcgggtgatcctggccgacgccaacctggacaaggtgctgagcgcctacaacaagcaccgggacaagcccatccgggagcaggccgagaacatcatccacctgttcaccctgaccaacctgggcgcccccgccgccttcaagtacttcgacaccaccatcgaccggaagcggtacaccagcaccaaggaggtgctggacgccaccctgatccaccagagcatcaccggcctgtacgagacccggatcgacctgagccagctgggcggcgacaagcggcccgccgccaccaagaaggccggccaggccaagaagaagaagggccgggccatcttcaagcccgaggagctgcggcaggccctgatgcccaccctggaggccctgtaccggcaggaccccgagagcctgcccttccggcagcccgtggacccccagctgctgggcatccccgactacttcgacatcgtgaaatcccccatggacctgagcaccatcaagcggaagctggacaccggccagtaccaggagccctggcagtacgtggacgacatctggctgatgttcaacaacgcctggctgtacaaccggaaaaccagccgggtgtacaagtactgcagcaagctgagcgaggtgttcgagcaggagatcgaccccgtgatgcagagcctgggctactgctgcggccggaagctggagttcagcccccagaccctgtgctgctacggcaagcagctgtgcaccatcccccgggacgccacctactacagctaccagaaccggtaccacttctgcgagaagtgcttcaacgagatccagggcgagagcgtgagcctgggcgacgaccccagccagccccagaccaccatcaacaaggagcagttcagcaagcggaagaacgacaccctggaccccgagctgttcgtggagtgcaccgagtgcggccggaagatgcaccagatctgcgtgctgcaccacgagatcatctggcccgccggcttcgtgtgcgacggctgcctgaagaaatccgcccggacccggaaggagaacaagttcagcgccaagcggctgcccagcacccggctgggcaccttcctggagaaccgggtgaacgacttcctgcggcggcagaaccaccccgagagcggcgaggtgaccgtgcgggtggtgcacgccagcgacaagaccgtggaggtgaagcccggcatgaaggcccggttcgtggacagcggcgagatggccgagagcttcccctaccggaccaaggccctgttcgccttcgaggagatcgacggcgtggacctgtgcttcttcggcatgcacgtgcaggagtacggcagcgactgccccccccccaaccagcggcgggtgtacatcagctacctggacagcgtgcacttcttccggcccaagtgcctgcggaccgccgtgtaccacgagatcctgatcggctacctggagtacgtgaagaagctgggctacaccaccggccacatctgggcctgcccccccagcgagggcgacgactacatcttccactgccacccccccgaccagaagatccccaagcccaagcggctgcaggagtggtacaagaagatgctggacaaggccgtgagcgagcggatcgtgcacgactacaaggacatcttcaagcaggccaccgaggaccggctgaccagcgccaaggagctgccctacttcgagggcgacttctggcccaacgtgctggaggagagcatcaaggagctggagcaggaggaggaggagcggaagcgggaggagaacaccagcaacgagagcaccgacgtgaccaagggcgacagcaagaacgccaagaagaagaacaacaagaaaaccagcaagaacaagagcagcctgagccggggcaacaagaagaagcccggcatgcccaacgtgagcaacgacctgagccagaagctgtacgccaccatggagaagcacaaggaggtgttcttcgtgatccggctgatcgccggccccgccgccaacagcctgccccccatcgtggaccccgaccccctgatcccctgcgacctgatggacggccgggacgccttcctgaccctggcccgggacaagcacctggagttcagcagcctgcggcgggcccagtggagcaccatgtgcatgctggtggagctgcacacccagagccaggacagcggcggcaagcggcccgccgccaccaagaaggccggccaggccaagaagaagaagggcagctacccctacgacgtgcccgactacgcctgagcggccgcttaattaagctgccttctgcggggcttgccttctggccatgcccttcttctctcccttgcacctgtacctcttggtctttgaataaagcctgagtaggaagtctagaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 8 - RNA, 6097 nt, synthetic construct
  misc_feature 1..6097: note = source = /note="Description of Artificial Sequence: Syntheticpolynucleotide"
  source 1..6097: mol_type = other RNA
aggaaataagagagaaaagaagagtaagaagaaatataagagccaccatggcccccaagaagaagcggaaggtgggcatccacggcgtgcccgccgccgacaagaagtacagcatcggcctggccatcggcaccaacagcgtgggctgggccgtgatcaccgacgagtacaaggtgcccagcaagaagttcaaggtgctgggcaacaccgaccggcacagcatcaagaagaacctgatcggcgccctgctgttcgacagcggcgagaccgccgaggccacccggctgaagcggaccgcccggcggcggtacacccggcggaagaaccggatctgctacctgcaggagatcttcagcaacgagatggccaaggtggacgacagcttcttccaccggctggaggagagcttcctggtggaggaggacaagaagcacgagcggcaccccatcttcggcaacatcgtggacgaggtggcctaccacgagaagtaccccaccatctaccacctgcggaagaagctggtggacagcaccgacaaggccgacctgcggctgatctacctggccctggcccacatgatcaagttccggggccacttcctgatcgagggcgacctgaaccccgacaacagcgacgtggacaagctgttcatccagctggtgcagacctacaaccagctgttcgaggagaaccccatcaacgccagcggcgtggacgccaaggccatcctgagcgcccggctgagcaagagccggcggctggagaacctgatcgcccagctgcccggcgagaagaagaacggcctgttcggcaacctgatcgccctgagcctgggcctgacccccaacttcaagagcaacttcgacctggccgaggacgccaagctgcagctgagcaaggacacctacgacgacgacctggacaacctgctggcccagatcggcgaccagtacgccgacctgttcctggccgccaagaacctgagcgacgccatcctgctgagcgacatcctgcgggtgaacaccgagatcaccaaggcccccctgagcgccagcatgatcaagcggtacgacgagcaccaccaggacctgaccctgctgaaggccctggtgcggcagcagctgcccgagaagtacaaggagatcttcttcgaccagagcaagaacggctacgccggctacatcgacggcggcgccagccaggaggagttctacaagttcatcaagcccatcctggagaagatggacggcaccgaggagctgctggtgaagctgaaccgggaggacctgctgcggaagcagcggaccttcgacaacggcagcatcccccaccagatccacctgggcgagctgcacgccatcctgcggcggcaggaggacttctaccccttcctgaaggacaaccgggagaagatcgagaagatcctgaccttccggatcccctactacgtgggccccctggcccggggcaacagccggttcgcctggatgacccggaaatccgaggagaccatcaccccctggaacttcgaggaggtggtggacaagggcgccagcgcccagagcttcatcgagcggatgaccaacttcgacaagaacctgcccaacgagaaggtgctgcccaagcacagcctgctgtacgagtacttcaccgtgtacaacgagctgaccaaggtgaagtacgtgaccgagggcatgcggaagcccgccttcctgagcggcgagcagaagaaggccatcgtggacctgctgttcaagaccaaccggaaggtgaccgtgaagcagctgaaggaggactacttcaagaagatcgagtgcttcgacagcgtggagatcagcggcgtggaggaccggttcaacgccagcctgggcacctaccacgacctgctgaagatcatcaaggacaaggacttcctggacaacgaggagaacgaggacatcctggaggacatcgtgctgaccctgaccctgttcgaggaccgggagatgatcgaggagcggctgaaaacctacgcccacctgttcgacgacaaggtgatgaagcagctgaagcggcggcggtacaccggctggggccggctgagccggaagctgatcaacggcatccgggacaagcagagcggcaagaccatcctggacttcctgaaatccgacggcttcgccaaccggaacttcatgcagctgatccacgacgacagcctgaccttcaaggaggacatccagaaggcccaggtgagcggccagggcgacagcctgcacgagcacatcgccaacctggccggcagccccgccatcaagaagggcatcctgcagaccgtgaaggtggtggacgagctggtgaaggtgatgggccggcacaagcccgagaacatcgtgatcgagatggcccgggagaaccagaccacccagaagggccagaagaacagccgggagcggatgaagcggatcgaggagggcatcaaggagctgggcagccagatcctgaaggagcaccccgtggagaacacccagctgcagaacgagaagctgtacctgtactacctgcagaacggccgggacatgtacgtggaccaggagctggacatcaaccggctgagcgactacgacgtggccgccatcgtgccccagagcttcctgaaggacgacagcatcgacaacaaggtgctgacccggagcgacaaggcccggggcaagagcgacaacgtgcccagcgaggaggtggtgaagaagatgaagaactactggcggcagctgctgaacgccaagctgatcacccagcggaagttcgacaacctgaccaaggccgagcggggcggcctgagcgagctggacaaggccggcttcatcaagcggcagctggtggagacccggcagatcaccaagcacgtggcccagatcctggacagccggatgaacaccaagtacgacgagaacgacaagctgatccgggaggtgaaggtgatcaccctgaaatccaagctggtgagcgacttccggaaggacttccagttctacaaggtgcgggagatcaacaactaccaccacgcccacgacgcctacctgaacgccgtggtgggcaccgccctgatcaagaagtaccccaagctggagagcgagttcgtgtacggcgactacaaggtgtacgacgtgcggaagatgatcgccaagagcgagcaggagatcggcaaggccaccgccaagtacttcttctacagcaacatcatgaacttcttcaagaccgagatcaccctggccaacggcgagatccggaagcggcccctgatcgagaccaacggcgagaccggcgagatcgtgtgggacaagggccgggacttcgccaccgtgcggaaggtgctgagcatgccccaggtgaacatcgtgaagaaaaccgaggtgcagaccggcggcttcagcaaggagagcatcctgcccaagcggaacagcgacaagctgatcgcccggaagaaggactgggaccccaagaagtacggcggcttcgacagccccaccgtggcctacagcgtgctggtggtggccaaggtggagaagggcaagagcaagaagctgaaatccgtgaaggagctgctgggcatcaccatcatggagcggagcagcttcgagaagaaccccatcgacttcctggaggccaagggctacaaggaggtgaagaaggacctgatcatcaagctgcccaagtacagcctgttcgagctggagaacggccggaagcggatgctggccagcgccggcgagctgcagaagggcaacgagctggccctgcccagcaagtacgtgaacttcctgtacctggccagccactacgagaagctgaagggcagccccgaggacaacgagcagaagcagctgttcgtggagcagcacaagcactacctggacgagatcatcgagcagatcagcgagttcagcaagcgggtgatcctggccgacgccaacctggacaaggtgctgagcgcctacaacaagcaccgggacaagcccatccgggagcaggccgagaacatcatccacctgttcaccctgaccaacctgggcgcccccgccgccttcaagtacttcgacaccaccatcgaccggaagcggtacaccagcaccaaggaggtgctggacgccaccctgatccaccagagcatcaccggcctgtacgagacccggatcgacctgagccagctgggcggcgacaagcggcccgccgccaccaagaaggccggccaggccaagaagaagaagggccgggccgacgccctggacgacttcgacctggacatgctgggcagcgacgccctggacgacttcgacctggacatgctgggcagcgacgccctggacgacttcgacctggacatgctgggcagcgacgccctggacgacttcgacctggacatgctgagcggcggccccaagaagaagcggaaggtgggcagccagtacctgcccgacaccgacgaccggcaccggatcgaggagaagcggaagcggacctacgagaccttcaagagcatcatgaagaaatcccccttcagcggccccaccgacccccggccccccccccggcggatcgccgtgcccagccggagcagcgccagcgtgcccaagcccgccccccagccctaccccttcaccagcagcctgagcaccatcaactacgacgagttccccaccatggtgttccccagcggccagatcagccaggccagcgccctggcccccgcccccccccaggtgctgccccaggcccccgcccccgcccccgcccccgccatggtgagcgccctggcccaggcccccgcccccgtgcccgtgctggcccccggccccccccaggccgtggccccccccgcccccaagcccacccaggccggcgagggcaccctgagcgaggccctgctgcagctgcagttcgacgacgaggacctgggcgccctgctgggcaacagcaccgaccccgccgtgttcaccgacctggccagcgtggacaacagcgagttccagcagctgctgaaccagggcatccccgtggccccccacaccaccgagcccatgctgatggagtaccccgaggccatcacccggctggtgaccggcgcccagcggccccccgaccccgcccccgcccccctgggcgcccccggcctgcccaacggcctgctgagcggcgacgaggacttcagcagcatcgccgacatggacttcagcgccctgctgggcagcggcagcggcagccgggacagccgggagggcatgttcctgcccaagcccgaggccggcagcgccatcagcgacgtgttcgagggccgggaggtgtgccagcccaagcggatccggcccttccacccccccggcagcccctgggccaaccggcccctgcccgccagcctggcccccacccccaccggccccgtgcacgagcccgtgggcagcctgacccccgcccccgtgccccagcccctggaccccgcccccgccgtgacccccgaggccagccacctgctggaggaccccgacgaggagaccagccaggccgtgaaggccctgcgggagatggccgacaccgtgatcccccagaaggaggaggccgccatctgcggccagatggacctgagccacccccccccccggggccacctggacgagctgaccaccaccctggagagcatgaccgaggacctgaacctggacagccccctgacccccgagctgaacgagatcctggacaccttcctgaacgacgagtgcctgctgcacgccatgcacatcagcaccggcctgagcatcttcgacaccagcctgttcagcggcggcaagcggcccgccgccaccaagaaggccggccaggccaagaagaagaagggcagctacccctacgacgtgcccgactacgcctgagcggccgcttaattaagctgccttctgcggggcttgccttctggccatgcccttcttctctcccttgcacctgtacctcttggtctttgaataaagcctgagtaggaagtctagaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 9 - protein, 1414 aa, synthetic construct
  REGION 1..1414: note = source = /note="Description of Artificial Sequence: Syntheticpolypeptide"
MAPKKKRKVGIHGVPAADKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVAAIVPQSFLKDDSIDNKVLTRSDKARGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGKRPAATKKAGQAKKKKGSYPYDVPDYA

SEQ ID NO: 10 - protein, 2050 aa, synthetic construct
  REGION 1..2050: note = source = /note="Description of Artificial Sequence: Syntheticpolypeptide"
MAPKKKRKVGIHGVPAADKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVAAIVPQSFLKDDSIDNKVLTRSDKARGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDKRPAATKKAGQAKKKKGRAIFKPEELRQALMPTLEALYRQDPESLPFRQPVDPQLLGIPDYFDIVKSPMDLSTIKRKLDTGQYQEPWQYVDDIWLMFNNAWLYNRKTSRVYKYCSKLSEVFEQEIDPVMQSLGYCCGRKLEFSPQTLCCYGKQLCTIPRDATYYSYQNRYHFCEKCFNEIQGESVSLGDDPSQPQTTINKEQFSKRKNDTLDPELFVECTECGRKMHQICVLHHEIIWPAGFVCDGCLKKSARTRKENKFSAKRLPSTRLGTFLENRVNDFLRRQNHPESGEVTVRVVHASDKTVEVKPGMKARFVDSGEMAESFPYRTKALFAFEEIDGVDLCFFGMHVQEYGSDCPPPNQRRVYISYLDSVHFFRPKCLRTAVYHEILIGYLEYVKKLGYTTGHIWACPPSEGDDYIFHCHPPDQKIPKPKRLQEWYKKMLDKAVSERIVHDYKDIFKQATEDRLTSAKELPYFEGDFWPNVLEESIKELEQEEEERKREENTSNESTDVTKGDSKNAKKKNNKKTSKNKSSLSRGNKKKPGMPNVSNDLSQKLYATMEKHKEVFFVIRLIAGPAANSLPPIVDPDPLIPCDLMDGRDAFLTLARDKHLEFSSLRRAQWSTMCMLVELHTQSQDSGGKRPAATKKAGQAKKKKGSYPYDVPDYA

SEQ ID NO: 11 - protein, 1951 aa, synthetic construct
  REGION 1..1951: note = source = /note="Description of Artificial Sequence: Syntheticpolypeptide"
MAPKKKRKVGIHGVPAADKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVAAIVPQSFLKDDSIDNKVLTRSDKARGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDKRPAATKKAGQAKKKKGRADALDDFDLDMLGSDALDDFDLDMLGSDALDDFDLDMLGSDALDDFDLDMLSGGPKKKRKVGSQYLPDTDDRHRIEEKRKRTYETFKSIMKKSPFSGPTDPRPPPRRIAVPSRSSASVPKPAPQPYPFTSSLSTINYDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLGSGSGSRDSREGMFLPKPEAGSAISDVFEGREVCQPKRIRPFHPPGSPWANRPLPASLAPTPTGPVHEPVGSLTPAPVPQPLDPAPAVTPEASHLLEDPDEETSQAVKALREMADTVIPQKEEAAICGQMDLSHPPPRGHLDELTTTLESMTEDLNLDSPLTPELNEILDTFLNDECLLHAMHISTGLSIFDTSLFSGGKRPAATKKAGQAKKKKGSYPYDVPDYA

SEQ ID NO: 12 - protein, 7 aa, synthetic construct
  REGION 1..7: note = source = /note="Description of Artificial Sequence: Syntheticpeptide"
RKDALRG

SEQ ID NO: 13 - protein, 7 aa, synthetic construct
  REGION 1..7: note = source = /note="Description of Artificial Sequence: Syntheticpeptide"
TTGALTE

SEQ ID NO: 14 - protein, 7 aa, synthetic construct
  REGION 1..7: note = source = /note="Description of Artificial Sequence: Syntheticpeptide"
QRHHLVE

SEQ ID NO: 15 - protein, 7 aa, synthetic construct
  REGION 1..7: note = source = /note="Description of Artificial Sequence: Syntheticpeptide"
QNSTLTE

SEQ ID NO: 16 - protein, 7 aa, synthetic construct
  REGION 1..7: note = source = /note="Description of Artificial Sequence: Syntheticpeptide"
RNDALTE

SEQ ID NO: 17 - protein, 7 aa, synthetic construct
  REGION 1..7: note = source = /note="Description of Artificial Sequence: Syntheticpeptide"
HKNALQN

SEQ ID NO: 18 - protein, 7 aa, synthetic construct
  REGION 1..7: note = source = /note="Description of Artificial Sequence: Syntheticpeptide"
RRSACRR

SEQ ID NO: 19 - protein, 7 aa, synthetic construct
  REGION 1..7: note = source = /note="Description of Artificial Sequence: Syntheticpeptide"
QKSSLIA

SEQ ID NO: 20 - protein, 7 aa, synthetic construct
  REGION 1..7: note = source = /note="Description of Artificial Sequence: Syntheticpeptide"
RRDELNV

SEQ ID NO: 21 - protein, 7 aa, synthetic construct
  REGION 1..7: note = source = /note="Description of Artificial Sequence: Syntheticpeptide"
TSGSLVR

SEQ ID NO: 22 - protein, 7 aa, synthetic construct
  REGION 1..7: note = source = /note="Description of Artificial Sequence: Syntheticpeptide"
DPGALVR

SEQ ID NO: 23 - protein, 7 aa, synthetic construct
  REGION 1..7: note = source = /note="Description of Artificial Sequence: Syntheticpeptide"
QSSSLVR

SEQ ID NO: 24 - protein, 7 aa, synthetic construct
  REGION 1..7: note = source = /note="Description of Artificial Sequence: Syntheticpeptide"
RSDELVR

SEQ ID NO: 25 - protein, 7 aa, synthetic construct
  REGION 1..7: note = source = /note="Description of Artificial Sequence: Syntheticpeptide"
RLRDIQF

SEQ ID NO: 26 - protein, 7 aa, synthetic construct
  REGION 1..7: note = source = /note="Description of Artificial Sequence: Syntheticpeptide"
RSDERKR

SEQ ID NO: 27 - protein, 7 aa, synthetic construct
  REGION 1..7: note = source = /note="Description of Artificial Sequence: Syntheticpeptide"
RSDHLTT

SEQ ID NO: 28 - protein, 7 aa, synthetic construct
  REGION 1..7: note = source = /note="Description of Artificial Sequence: Syntheticpeptide"
RLRALDR

SEQ ID NO: 29 - protein, 7 aa, synthetic construct
  REGION 1..7: note = source = /note="Description of Artificial Sequence: Syntheticpeptide"
TKNSLTE

SEQ ID NO: 30 - protein, 7 aa, synthetic construct
  REGION 1..7: note = source = /note="Description of Artificial Sequence: Syntheticpeptide"
SKKHLAE

SEQ ID NO: 31 - protein, 7 aa, synthetic construct
  REGION 1..7: note = source = /note="Description of Artificial Sequence: Syntheticpeptide"
TSHSLTE

SEQ ID NO: 32 - protein, 7 aa, synthetic construct
  REGION 1..7: note = source = /note="Description of Artificial Sequence: Syntheticpeptide"
RNDTLTE

SEQ ID NO: 33 - protein, 7 aa, synthetic construct
  REGION 1..7: note = source = /note="Description of Artificial Sequence: Syntheticpeptide"
THLDLIR

SEQ ID NO: 34 - protein, 7 aa, synthetic construct
  REGION 1..7: note = source = /note="Description of Artificial Sequence: Syntheticpeptide"
DKKDLTR

SEQ ID NO: 35 - protein, 7 aa, synthetic construct
  REGION 1..7: note = source = /note="Description of Artificial Sequence: Syntheticpeptide"
SPADLTR

SEQ ID NO: 36 - protein, 7 aa, synthetic construct
  REGION 1..7: note = source = /note="Description of Artificial Sequence: Syntheticpeptide"
RTDTLRD

SEQ ID NO: 37 - protein, 7 aa, synthetic construct
  REGION 1..7: note = source = /note="Description of Artificial Sequence: Syntheticpeptide"
TSGELVR

SEQ ID NO: 38 - protein, 7 aa, synthetic construct
  REGION 1..7: note = source = /note="Description of Artificial Sequence: Syntheticpeptide"
DCRDLAR

SEQ ID NO: 39 - protein, 7 aa, synthetic construct
  REGION 1..7: note = source = /note="Description of Artificial Sequence: Syntheticpeptide"
QSGDLRR

SEQ ID NO: 40 - protein, 7 aa, synthetic construct
  REGION 1..7: note = source = /note="Description of Artificial Sequence: Syntheticpeptide"
RSDDLVR

SEQ ID NO: 41 - protein, 7 aa, synthetic construct
  REGION 1..7: note = source = /note="Description of Artificial Sequence: Syntheticpeptide"
ARGNLRT

SEQ ID NO: 42 - protein, 7 aa, synthetic construct
  REGION 1..7: note = source = /note="Description of Artificial Sequence: Syntheticpeptide"
SRGNLKS

SEQ ID NO: 43 - protein, 7 aa, synthetic construct
  REGION 1..7: note = source = /note="Description of Artificial Sequence: Syntheticpeptide"
QASNLIS

SEQ ID NO: 44 - protein, 7 aa, synthetic construct
  REGION 1..7: note = source = /note="Description of Artificial Sequence: Syntheticpeptide"
REDNLHT

SEQ ID NO: 45 - protein, 7 aa, synthetic construct
  REGION 1..7: note = source = /note="Description of Artificial Sequence: Syntheticpeptide"
TSGNLTE

SEQ ID NO: 46 - protein, 7 aa, synthetic construct
  REGION 1..7: note = source = /note="Description of Artificial Sequence: Syntheticpeptide"
SKKALTE

SEQ ID NO: 47 - protein, 7 aa, synthetic construct
  REGION 1..7: note = source = /note="Description of Artificial Sequence: Syntheticpeptide"
QSGNLTE

SEQ ID NO: 48 - protein, 7 aa, synthetic construct
  REGION 1..7: note = source = /note="Description of Artificial Sequence: Syntheticpeptide"
RADNLTE

SEQ ID NO: 49 - protein, 7 aa, synthetic construct
  REGION 1..7: note = source = /note="Description of Artificial Sequence: Syntheticpeptide"
TTGNLTV

SEQ ID NO: 50 - protein, 7 aa, synthetic construct
  REGION 1..7: note = source = /note="Description of Artificial Sequence: Syntheticpeptide"
DSGNLRV

SEQ ID NO: 51 - protein, 7 aa, synthetic construct
  REGION 1..7: note = source = /note="Description of Artificial Sequence: Syntheticpeptide"
QRANLRA

SEQ ID NO: 52 - protein, 7 aa, synthetic construct
  REGION 1..7: note = source = /note="Description of Artificial Sequence: Syntheticpeptide"
RKDNLKN

SEQ ID NO: 53 - protein, 7 aa, synthetic construct
  REGION 1..7: note = source = /note="Description of Artificial Sequence: Syntheticpeptide"
TSGNLVR

SEQ ID NO: 54 - protein, 7 aa, synthetic construct
  REGION 1..7: note = source = /note="Description of Artificial Sequence: Syntheticpeptide"
DPGNLVR

SEQ ID NO: 55 - protein, 7 aa, synthetic construct
  REGION 1..7: note = source = /note="Description of Artificial Sequence: Syntheticpeptide"
QSSNLVR

SEQ ID NO: 56 - protein, 7 aa, synthetic construct
  REGION 1..7: note = source = /note="Description of Artificial Sequence: Syntheticpeptide"
RSDNLVR

SEQ ID NO: 57 - protein, 7 aa, synthetic construct
  REGION 1..7: note = source = /note="Description of Artificial Sequence: Syntheticpeptide"
APKALGW

SEQ ID NO: 58 - protein, 7 aa, synthetic construct
  REGION 1..7: note = source = /note="Description of Artificial Sequence: Syntheticpeptide"
QAGHLAS

SEQ ID NO: 59 - protein, 7 aa, synthetic construct
  REGION 1..7: note = source = /note="Description of Artificial Sequence: Syntheticpeptide"
SRRTCRA

SEQ ID NO: 60 - protein, 7 aa, synthetic construct
  REGION 1..7: note = source = /note="Description of Artificial Sequence: Syntheticpeptide"
HTGHLLE

SEQ ID NO: 61 - protein, 11 aa, synthetic construct
  REGION 1..11: note = source = /note="Description of Artificial Sequence: Syntheticpeptide"
YKCPECGKSFS

SEQ ID NO: 62 - protein, 5 aa, synthetic construct
  REGION 1..5: note = source = /note="Description of Artificial Sequence: Syntheticpeptide"
HQRTH

SEQ ID NO: 63 - protein, 5 aa, synthetic construct
  REGION 1..5: note = source = /note="Description of Artificial Sequence: Syntheticpeptide"
TGEKP

SEQ ID NO: 64 - protein, 518 aa, synthetic construct
  REGION 1..518: note = source = /note="Description of Artificial Sequence: Syntheticpolypeptide"
DALDDFDLDMLGSDALDDFDLDMLGSDALDDFDLDMLGSDALDDFDLDMLSGGPKKKRKVGSQYLPDTDDRHRIEEKRKRTYETFKSIMKKSPFSGPTDPRPPPRRIAVPSRSSASVPKPAPQPYPFTSSLSTINYDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLGSGSGSRDSREGMFLPKPEAGSAISDVFEGREVCQPKRLRPFHPPGSPWANRPLPASLAPTPTGPVHEPVGSLTPAPVPQPLDPAPAVTPEASHLLEDPDEETSQAVKALREMADTVIPQKEEAAICGQMDLSHPPPRGHLDELTTTLESMTEDLNLDSPLTPELNEILDTFLNDECLLHAMHISTGLSIFDTSLF

SEQ ID NO: 65 - protein, 617 aa, Homo sapiens
IFKPEELRQALMPTLEALYRQDPESLPFRQPVDPQLLGIPDYFDIVKSPMDLSTIKRKLDTGQYQEPWQYVDDIWLMFNNAWLYNRKTSRVYKYCSKLSEVFEQEIDPVMQSLGYCCGRKLEFSPQTLCCYGKQLCTIPRDATYYSYQNRYHFCEKCFNEIQGESVSLGDDPSQPQTTINKEQFSKRKNDTLDPELFVECTECGRKMHQICVLHHEIIWPAGFVCDGCLKKSARTRKENKFSAKRLPSTRLGTFLENRVNDFLRRQNHPESGEVTVRVVHASDKTVEVKPGMKARFVDSGEMAESFPYRTKALFAFEEIDGVDLCFFGMHVQEYGSDCPPPNQRRVYISYLDSVHFFRPKCLRTAVYHEILIGYLEYVKKLGYTTGHIWACPPSEGDDYIFHCHPPDQKIPKPKRLQEWYKKMLDKAVSERIVHDYKDIFKQATEDRLTSAKELPYFEGDFWPNVLEESIKELEQEEEERKREENTSNESTDVTKGDSKNAKKKNNKKTSKNKSSLSRGNKKKPGMPNVSNDLSQKLYATMEKHKEVFFVIRLIAGPAANSLPPIVDPDPLIPCDLMDGRDAFLTLARDKHLEFSSLRRAQWSTMCMLVELHTQSQD

SEQ ID NO: 66 - protein, 7 aa, synthetic construct
  REGION 1..7: note = source = /note="Description of Artificial Sequence: Syntheticpeptide"
QSGHLTE

SEQ ID NO: 67 - protein, 7 aa, synthetic construct
  REGION 1..7: note = source = /note="Description of Artificial Sequence: Syntheticpeptide"
RSDKLTE

SEQ ID NO: 68 - protein, 7 aa, synthetic construct
  REGION 1..7: note = source = /note="Description of Artificial Sequence: Syntheticpeptide"
HRTTLTN

SEQ ID NO: 69 - protein, 7 aa, synthetic construct
  REGION 1..7: note = source = /note="Description of Artificial Sequence: Syntheticpeptide"
ERSHLRE

SEQ ID NO: 70 - protein, 7 aa, synthetic construct
  REGION 1..7: note = source = /note="Description of Artificial Sequence: Syntheticpeptide"
QLAHLRA

SEQ ID NO: 71 - protein, 7 aa, synthetic construct
  REGION 1..7: note = source = /note="Description of Artificial Sequence: Syntheticpeptide"
RSDHLTN

SEQ ID NO: 72 - protein, 7 aa, synthetic construct
  REGION 1..7: note = source = /note="Description of Artificial Sequence: Syntheticpeptide"
TSGHLVR

SEQ ID NO: 73 - protein, 7 aa, synthetic construct
  REGION 1..7: note = source = /note="Description of Artificial Sequence: Syntheticpeptide"
DPGHLVR

SEQ ID NO: 74 - protein, 7 aa, synthetic construct
  REGION 1..7: note = source = /note="Description of Artificial Sequence: Syntheticpeptide"
QRAHLER

SEQ ID NO: 75 - protein, 7 aa, synthetic construct
  REGION 1..7: note = source = /note="Description of Artificial Sequence: Syntheticpeptide"
RSDKLVR

SEQ ID NO: 76 - protein, 7 aa, synthetic construct
  REGION 1..7: note = source = /note="Description of Artificial Sequence: Syntheticpeptide"
LEPGEKP

SEQ ID NO: 77 - protein, 6 aa, synthetic construct
  REGION 1..6: note = source = /note="Description of Artificial Sequence: Syntheticpeptide"
TGKKTS

SEQ ID NO: 78 - protein, 12 aa, synthetic construct
  REGION 1..12: note = source = /note="Description of Artificial Sequence: Syntheticpeptide"
THPRAPIPKPFQ

SEQ ID NO: 79 - protein, 16 aa, synthetic construct
  REGION 1..16: note = source = /note="Description of Artificial Sequence: Syntheticpeptide"
TPNPHRRTDPSHKPFQ

SEQ ID NO: 80 - DNA, 22 bp, Homo sapiens
  source 1..22: mol_type = genomic DNA
cactcaccttggtgaagtggac

SEQ ID NO: 81 - DNA, 22 bp, Homo sapiens
  source 1..22: mol_type = genomic DNA
accttggtgaagtggactgaca

SEQ ID NO: 82 - DNA, 21 bp, Homo sapiens
  source 1..21: mol_type = genomic DNA
ccttggtgaagtggactgaca

SEQ ID NO: 83 - DNA, 21 bp, Homo sapiens
  source 1..21: mol_type = genomic DNA
tggtgaagtggactgacagaa

SEQ ID NO: 84 - DNA, 21 bp, Homo sapiens
  source 1..21: mol_type = genomic DNA
tgaagtggactgacagaaaag

SEQ ID NO: 85 - DNA, 21 bp, Homo sapiens
  source 1..21: mol_type = genomic DNA
agtggactgacagaaaaggat

SEQ ID NO: 86 - DNA, 21 bp, Homo sapiens
  source 1..21: mol_type = genomic DNA
ggactgacagaaaaggatcag

SEQ ID NO: 87 - DNA, 21 bp, Homo sapiens
  source 1..21: mol_type = genomic DNA
ctgacagaaaaggatcagcct

SEQ ID NO: 88 - DNA, 21 bp, Homo sapiens
  source 1..21: mol_type = genomic DNA
acagaaaaggatcagcctggc

SEQ ID NO: 89 - DNA, 23 bp, Homo sapiens
  source 1..23: mol_type = genomic DNA
gaaaaggatcagcctggcttgtg

SEQ ID NO: 90 - DNA, 23 bp, Homo sapiens
  source 1..23: mol_type = genomic DNA
aaggatcagcctggcttgtggga

SEQ ID NO: 91 - DNA, 23 bp, Homo sapiens
  source 1..23: mol_type = genomic DNA
gatcagcctggcttgtgggaaac

SEQ ID NO: 92 - DNA, 24 bp, Homo sapiens
  source 1..24: mol_type = genomic DNA
cagcctggcttgtgggaaactgtc

SEQ ID NO: 93 - DNA, 24 bp, Homo sapiens
  source 1..24: mol_type = genomic DNA
cctggcttgtgggaaactgtcacg

SEQ ID NO: 94 - DNA, 21 bp, Homo sapiens
  source 1..21: mol_type = genomic DNA
gtgaagtggactgacagaaaa

SEQ ID NO: 95 - protein, 11 aa, Human alphaherpesvirus 1
DALDDFDLDML

SEQ ID NO: 96 - RNA, 100 nt, synthetic construct
  misc_feature 1..100: note = source = /note="Description of Artificial Sequence: Syntheticpolynucleotide"
  source 1..100: mol_type = other RNA
tctgtcagtccacttcaccagttttagagctagaaatagcaagttaaaataaggctagtccgttatcaacttgaaaaagtggcaccgagtcggtgctttt

SEQ ID NO: 97 - DNA, 100 bp, synthetic construct
  misc_feature 1..100: note = source = /note="Description of Artificial Sequence: Syntheticpolynucleotide"
  source 1..100: mol_type = other DNA
tctgtcagtccacttcaccagttttagagctagaaatagcaagttaaaataaggctagtccgttatcaacttgaaaaagtggcaccgagtcggtgctttt

SEQ ID NO: 98 - RNA, 100 nt, synthetic construct
  misc_feature 1..100: note = source = /note="Description of Artificial Sequence: Syntheticpolynucleotide"
  source 1..100: mol_type = other RNA
gtgacagtttcccacaagccgttttagagctagaaatagcaagttaaaataaggctagtccgttatcaacttgaaaaagtggcaccgagtcggtgctttt

SEQ ID NO: 99 - DNA, 100 bp, synthetic construct
  misc_feature 1..100: note = source = /note="Description of Artificial Sequence: Syntheticpolynucleotide"
  source 1..100: mol_type = other DNA
gtgacagtttcccacaagccgttttagagctagaaatagcaagttaaaataaggctagtccgttatcaacttgaaaaagtggcaccgagtcggtgctttt

SEQ ID NO: 100 - RNA, 100 nt, synthetic construct
  misc_feature 1..100: note = source = /note="Description of Artificial Sequence: Syntheticpolynucleotide"
  source 1..100: mol_type = other RNA
aaaaaccacgctgtacggtggttttagagctagaaatagcaagttaaaataaggctagtccgttatcaacttgaaaaagtggcaccgagtcggtgctttt

SEQ ID NO: 101 - DNA, 100 bp, synthetic construct
  misc_feature 1..100: note = source = /note="Description of Artificial Sequence: Syntheticpolynucleotide"
  source 1..100: mol_type = other DNA
aaaaaccacgctgtacggtggttttagagctagaaatagcaagttaaaataaggctagtccgttatcaacttgaaaaagtggcaccgagtcggtgctttt

SEQ ID NO: 102 - RNA, 100 nt, synthetic construct
  misc_feature 1..100: note = source = /note="Description of Artificial Sequence: Syntheticpolynucleotide"
  source 1..100: mol_type = other RNA
tgtgtgcgctgataatcacggttttagagctagaaatagcaagttaaaataaggctagtccgttatcaacttgaaaaagtggcaccgagtcggtgctttt

SEQ ID NO: 103 - DNA, 100 bp, synthetic construct
  misc_feature 1..100: note = source = /note="Description of Artificial Sequence: Syntheticpolynucleotide"
  source 1..100: mol_type = other DNA
tgtgtgcgctgataatcacggttttagagctagaaatagcaagttaaaataaggctagtccgttatcaacttgaaaaagtggcaccgagtcggtgctttt

SEQ ID NO: 104 - RNA, 100 nt, synthetic construct
  misc_feature 1..100: note = source = /note="Description of Artificial Sequence: Syntheticpolynucleotide"
  source 1..100: mol_type = other RNA
taagtctcataatcaagaaagttttagagctagaaatagcaagttaaaataaggctagtccgttatcaacttgaaaaagtggcaccgagtcggtgctttt

SEQ ID NO: 105 - DNA, 100 bp, synthetic construct
  misc_feature 1..100: note = source = /note="Description of Artificial Sequence: Syntheticpolynucleotide"
  source 1..100: mol_type = other DNA
taagtctcataatcaagaaagttttagagctagaaatagcaagttaaaataaggctagtccgttatcaacttgaaaaagtggcaccgagtcggtgctttt

SEQ ID NO: 106 - RNA, 100 nt, synthetic construct
  misc_feature 1..100: note = source = /note="Description of Artificial Sequence: Syntheticpolynucleotide"
  source 1..100: mol_type = other RNA
tattttcagatgactcgtaagttttagagctagaaatagcaagttaaaataaggctagtccgttatcaacttgaaaaagtggcaccgagtcggtgctttt

SEQ ID NO: 107 - DNA, 100 bp, synthetic construct
  misc_feature 1..100: note = source = /note="Description of Artificial Sequence: Syntheticpolynucleotide"
  source 1..100: mol_type = other DNA
tattttcagatgactcgtaagttttagagctagaaatagcaagttaaaataaggctagtccgttatcaacttgaaaaagtggcaccgagtcggtgctttt

SEQ ID NO: 108 - RNA, 100 nt, synthetic construct
  misc_feature 1..100: note = source = /note="Description of Artificial Sequence: Syntheticpolynucleotide"
  source 1..100: mol_type = other RNA
taaatcacagggccaacccggttttagagctagaaatagcaagttaaaataaggctagtccgttatcaacttgaaaaagtggcaccgagtcggtgctttt

SEQ ID NO: 109 - DNA, 100 bp, synthetic construct
  misc_feature 1..100: note = source = /note="Description of Artificial Sequence: Syntheticpolynucleotide"
  source 1..100: mol_type = other DNA
taaatcacagggccaacccggttttagagctagaaatagcaagttaaaataaggctagtccgttatcaacttgaaaaagtggcaccgagtcggtgctttt

SEQ ID NO: 110 - RNA, 100 nt, synthetic construct
  misc_feature 1..100: note = source = /note="Description of Artificial Sequence: Syntheticpolynucleotide"
  source 1..100: mol_type = other RNA
gacaccattctgtgagtgaggttttagagctagaaatagcaagttaaaataaggctagtccgttatcaacttgaaaaagtggcaccgagtcggtgctttt

SEQ ID NO: 111 - DNA, 100 bp, synthetic construct
  misc_feature 1..100: note = source = /note="Description of Artificial Sequence: Syntheticpolynucleotide"
  source 1..100: mol_type = other DNA
gacaccattctgtgagtgaggttttagagctagaaatagcaagttaaaataaggctagtccgttatcaacttgaaaaagtggcaccgagtcggtgctttt

SEQ ID NO: 112 - RNA, 100 nt, synthetic construct
  misc_feature 1..100: note = source = /note="Description of Artificial Sequence: Syntheticpolynucleotide"
  source 1..100: mol_type = other RNA
agaatctgaagctctatgtggttttagagctagaaatagcaagttaaaataaggctagtccgttatcaacttgaaaaagtggcaccgagtcggtgctttt

SEQ ID NO: 113 - DNA, 100 bp, synthetic construct
  misc_feature 1..100: note = source = /note="Description of Artificial Sequence: Syntheticpolynucleotide"
  source 1..100: mol_type = other DNA
agaatctgaagctctatgtggttttagagctagaaatagcaagttaaaataaggctagtccgttatcaacttgaaaaagtggcaccgagtcggtgctttt

SEQ ID NO: 114 - DNA, 20 bp, synthetic construct
  misc_feature 1..20: note = source = /note="Description of Artificial Sequence: Syntheticoligonucleotide"
  source 1..20: mol_type = other DNA
tctgtcagtccacttcacca

SEQ ID NO: 115 - DNA, 20 bp, Homo sapiens
  source 1..20: mol_type = genomic DNA
tggtgaagtggactgacaga

SEQ ID NO: 116 - DNA, 20 bp, synthetic construct
  misc_feature 1..20: note = source = /note="Description of Artificial Sequence: Syntheticoligonucleotide"
  source 1..20: mol_type = other DNA
gtgacagtttcccacaagcc

SEQ ID NO: 117 - DNA, 20 bp, Homo sapiens
  source 1..20: mol_type = genomic DNA
ggcttgtgggaaactgtcac

SEQ ID NO: 118 - DNA, 20 bp, synthetic construct
  misc_feature 1..20: note = source = /note="Description of Artificial Sequence: Syntheticoligonucleotide"
  source 1..20: mol_type = other DNA
aaaaaccacgctgtacggtg

SEQ ID NO: 119 - DNA, 20 bp, Homo sapiens
  source 1..20: mol_type = genomic DNA
caccgtacagcgtggttttt

SEQ ID NO: 120 - DNA, 20 bp, synthetic construct
  misc_feature 1..20: note = source = /note="Description of Artificial Sequence: Syntheticoligonucleotide"
  source 1..20: mol_type = other DNA
tgtgtgcgctgataatcacg

SEQ ID NO: 121 - DNA, 20 bp, Homo sapiens
  source 1..20: mol_type = genomic DNA
cgtgattatcagcgcacaca

SEQ ID NO: 122 - DNA, 20 bp, synthetic construct
  misc_feature 1..20: note = source = /note="Description of Artificial Sequence: Syntheticoligonucleotide"
  source 1..20: mol_type = other DNA
taagtctcataatcaagaaa

SEQ ID NO: 123 - DNA, 20 bp, Homo sapiens
  source 1..20: mol_type = genomic DNA
tttcttgattatgagactta

SEQ ID NO: 124 - DNA, 20 bp, synthetic construct
  misc_feature 1..20: note = source = /note="Description of Artificial Sequence: Syntheticoligonucleotide"
  source 1..20: mol_type = other DNA
tattttcagatgactcgtaa

SEQ ID NO: 125 - DNA, 20 bp, Homo sapiens
  source 1..20: mol_type = genomic DNA
ttacgagtcatctgaaaata

SEQ ID NO: 126 - DNA, 20 bp, synthetic construct
  misc_feature 1..20: note = source = /note="Description of Artificial Sequence: Syntheticoligonucleotide"
  source 1..20: mol_type = other DNA
taaatcacagggccaacccg

SEQ ID NO: 127 - DNA, 20 bp, Homo sapiens
  source 1..20: mol_type = genomic DNA
cgggttggccctgtgattta

SEQ ID NO: 128 - DNA, 20 bp, synthetic construct
  misc_feature 1..20: note = source = /note="Description of Artificial Sequence: Syntheticoligonucleotide"
  source 1..20: mol_type = other DNA
gacaccattctgtgagtgag

SEQ ID NO: 129 - DNA, 20 bp, Homo sapiens
  source 1..20: mol_type = genomic DNA
ctcactcacagaatggtgtc

SEQ ID NO: 130 - DNA, 20 bp, synthetic construct
  misc_feature 1..20: note = source = /note="Description of Artificial Sequence: Syntheticoligonucleotide"
  source 1..20: mol_type = other DNA
agaatctgaagctctatgtg

SEQ ID NO: 131 - DNA, 20 bp, Homo sapiens
  source 1..20: mol_type = genomic DNA
cacatagagcttcagattct

SEQ ID NO: 132 - DNA, 80 bp, synthetic construct
  misc_feature 1..80: note = source = /note="Description of Artificial Sequence: Syntheticoligonucleotide"
  source 1..80: mol_type = other DNA
gttttagagctagaaatagcaagttaaaataaggctagtccgttatcaacttgaaaaagtggcaccgagtcggtgctttt

SEQ ID NO: 133 - DNA, 20 bp, Homo sapiens
  source 1..20: mol_type = genomic DNA
tctgtcagtccacttcacca

SEQ ID NO: 134 - DNA, 20 bp, Homo sapiens
  source 1..20: mol_type = genomic DNA
gtgacagtttcccacaagcc

SEQ ID NO: 135 - DNA, 20 bp, Homo sapiens
  source 1..20: mol_type = genomic DNA
aaaaaccacgctgtacggtg

SEQ ID NO: 136 - DNA, 20 bp, Homo sapiens
  source 1..20: mol_type = genomic DNA
tgtgtgcgctgataatcacg

SEQ ID NO: 137 - DNA, 20 bp, Homo sapiens
  source 1..20: mol_type = genomic DNA
taagtctcataatcaagaaa

SEQ ID NO: 138 - DNA, 20 bp, Homo sapiens
  source 1..20: mol_type = genomic DNA
tattttcagatgactcgtaa

SEQ ID NO: 139 - DNA, 20 bp, Homo sapiens
  source 1..20: mol_type = genomic DNA
taaatcacagggccaacccg

SEQ ID NO: 140 - DNA, 20 bp, Homo sapiens
  source 1..20: mol_type = genomic DNA
gacaccattctgtgagtgag

SEQ ID NO: 141 - DNA, 20 bp, Homo sapiens
  source 1..20: mol_type = genomic DNA
agaatctgaagctctatgtg

SEQ ID NO: 142 - DNA, 21 bp, Homo sapiens
  source 1..21: mol_type = genomic DNA
aagtggactgacagaaaagga

SEQ ID NO: 143 - DNA, 22 bp, Homo sapiens
  source 1..22: mol_type = genomic DNA
tggactgacagaaaaggatcag

SEQ ID NO: 144 - DNA, 22 bp, Homo sapiens
  source 1..22: mol_type = genomic DNA
actgacagaaaaggatcagcct

SEQ ID NO: 145 - DNA, 22 bp, Homo sapiens
  source 1..22: mol_type = genomic DNA
gacagaaaaggatcagcctggc

SEQ ID NO: 146 - DNA, 24 bp, Homo sapiens
  source 1..24: mol_type = genomic DNA
agaaaaggatcagcctggcttgtg

SEQ ID NO: 147 - DNA, 24 bp, Homo sapiens
  source 1..24: mol_type = genomic DNA
aaaggatcagcctggcttgtggga

SEQ ID NO: 148 - DNA, 24 bp, Homo sapiens
  source 1..24: mol_type = genomic DNA
ggatcagcctggcttgtgggaaac

SEQ ID NO: 149 - DNA, 23 bp, Homo sapiens
  source 1..23: mol_type = genomic DNA
agcctggcttgtgggaaactgtc

SEQ ID NO: 150 - DNA, 23 bp, Homo sapiens
  source 1..23: mol_type = genomic DNA
ctggcttgtgggaaactgtcacg

SEQ ID NO: 151 - DNA, 22 bp, Homo sapiens
  source 1..22: mol_type = genomic DNA
gcctggcttgtgggaaactgtc

SEQ ID NO: 152 - DNA, 22 bp, Homo sapiens
  source 1..22: mol_type = genomic DNA
tggcttgtgggaaactgtcacg

SEQ ID NO: 153 - DNA, 23 bp, Homo sapiens
  source 1..23: mol_type = genomic DNA
gggaaactgtcacgtatcaaaaa

SEQ ID NO: 154 - DNA, 23 bp, Homo sapiens
  source 1..23: mol_type = genomic DNA
aaactgtcacgtatcaaaaacaa

SEQ ID NO: 155 - DNA, 23 bp, Homo sapiens
  source 1..23: mol_type = genomic DNA
ctgtcacgtatcaaaaacaactt

SEQ ID NO: 156 - DNA, 23 bp, Homo sapiens
  source 1..23: mol_type = genomic DNA
cacgtatcaaaaacaactttgct

SEQ ID NO: 157 - DNA, 23 bp, Homo sapiens
  source 1..23: mol_type = genomic DNA
cttttataccgagaagaaaaacc

SEQ ID NO: 158 - DNA, 21 bp, Homo sapiens
  source 1..21: mol_type = genomic DNA
ataccgagaagaaaaaccacg

SEQ ID NO: 159 - DNA, 21 bp, Homo sapiens
  source 1..21: mol_type = genomic DNA
ccgagaagaaaaaccacgctg

SEQ ID NO: 160 - DNA, 22 bp, Homo sapiens
  source 1..22: mol_type = genomic DNA
agaagaaaaaccacgctgtacg

SEQ ID NO: 161 - DNA, 22 bp, Homo sapiens
  source 1..22: mol_type = genomic DNA
agaaaaaccacgctgtacggtg

SEQ ID NO: 162 - DNA, 22 bp, Homo sapiens
  source 1..22: mol_type = genomic DNA
aaaaccacgctgtacggtgtgg

SEQ ID NO: 163 - DNA, 22 bp, Homo sapiens
  source 1..22: mol_type = genomic DNA
accacgctgtacggtgtggaag

SEQ ID NO: 164 - DNA, 22 bp, Homo sapiens
  source 1..22: mol_type = genomic DNA
acgctgtacggtgtggaagccg

SEQ ID NO: 165 - DNA, 22 bp, Homo sapiens
  source 1..22: mol_type = genomic DNA
ctgtacggtgtggaagccgcag

SEQ ID NO: 166 - DNA, 21 bp, Homo sapiens
  source 1..21: mol_type = genomic DNA
accgagaagaaaaaccacgct

SEQ ID NO: 167 - DNA, 21 bp, Homo sapiens
  source 1..21: mol_type = genomic DNA
gagaagaaaaaccacgctgta

SEQ ID NO: 168 - DNA, 21 bp, Homo sapiens
  source 1..21: mol_type = genomic DNA
aagaaaaaccacgctgtacgg

SEQ ID NO: 169 - DNA, 22 bp, Homo sapiens
  source 1..22: mol_type = genomic DNA
aaaaaccacgctgtacggtgtg

SEQ ID NO: 170 - DNA, 22 bp, Homo sapiens
  source 1..22: mol_type = genomic DNA
aaccacgctgtacggtgtggaa

SEQ ID NO: 171 - DNA, 22 bp, Homo sapiens
  source 1..22: mol_type = genomic DNA
cacgctgtacggtgtggaagcc

SEQ ID NO: 172 - DNA, 22 bp, Homo sapiens
  source 1..22: mol_type = genomic DNA
gctgtacggtgtggaagccgca

SEQ ID NO: 173 - DNA, 22 bp, Homo sapiens
  source 1..22: mol_type = genomic DNA
gtacggtgtggaagccgcagac

SEQ ID NO: 174 - DNA, 22 bp, Homo sapiens
  source 1..22: mol_type = genomic DNA
cgagaagaaaaaccacgctgta

SEQ ID NO: 175 - DNA, 22 bp, Homo sapiens
  source 1..22: mol_type = genomic DNA
gaagaaaaaccacgctgtacgg

SEQ ID NO: 176 - DNA, 23 bp, Homo sapiens
  source 1..23: mol_type = genomic DNA
gaaaaaccacgctgtacggtgtg

SEQ ID NO: 177 - DNA, 23 bp, Homo sapiens
  source 1..23: mol_type = genomic DNA
aaaccacgctgtacggtgtggaa

SEQ ID NO: 178 - DNA, 23 bp, Homo sapiens
  source 1..23: mol_type = genomic DNA
ccacgctgtacggtgtggaagcc

SEQ ID NO: 179 - DNA, 23 bp, Homo sapiens
  source 1..23: mol_type = genomic DNA
cgctgtacggtgtggaagccgca

SEQ ID NO: 180 - DNA, 21 bp, Homo sapiens
  source 1..21: mol_type = genomic DNA
acggtgtggaagccgcagacc

SEQ ID NO: 181 - DNA, 23 bp, Homo sapiens
  source 1..23: mol_type = genomic DNA
cgatgagtgtgtgcgctgataat

SEQ ID NO: 182 - DNA, 23 bp, Homo sapiens
  source 1..23: mol_type = genomic DNA
tgagtgtgtgcgctgataatcac

SEQ ID NO: 183 - DNA, 23 bp, Homo sapiens
  source 1..23: mol_type = genomic DNA
gtgtgtgcgctgataatcacggg

SEQ ID NO: 184 - DNA, 25 bp, Homo sapiens
  source 1..25: mol_type = genomic DNA
gatgagtgtgtgcgctgataatcac

SEQ ID NO: 185 - DNA, 25 bp, Homo sapiens
  source 1..25: mol_type = genomic DNA
gagtgtgtgcgctgataatcacggg

SEQ ID NO: 186 - DNA, 24 bp, Homo sapiens
  source 1..24: mol_type = genomic DNA
atgagtgtgtgcgctgataatcac

SEQ ID NO: 187 - DNA, 24 bp, Homo sapiens
  source 1..24: mol_type = genomic DNA
agtgtgtgcgctgataatcacggg

SEQ ID NO: 188 - DNA, 24 bp, Homo sapiens
  source 1..24: mol_type = genomic DNA
gtgtgcgctgataatcacggggtg

SEQ ID NO: 189 - DNA, 22 bp, Homo sapiens
  source 1..22: mol_type = genomic DNA
gtgcgctgataatcacggggtg

SEQ ID NO: 190 - DNA, 22 bp, Homo sapiens
  source 1..22: mol_type = genomic DNA
cgctgataatcacggggtgggg

SEQ ID NO: 191 - DNA, 22 bp, Homo sapiens
  source 1..22: mol_type = genomic DNA
tgataatcacggggtggggggg

SEQ ID NO: 192 - DNA, 23 bp, Homo sapiens
  source 1..23: mol_type = genomic DNA
gcgctgataatcacggggtgggg

SEQ ID NO: 193 - DNA, 23 bp, Homo sapiens
  source 1..23: mol_type = genomic DNA
ctgataatcacggggtggggggg

SEQ ID NO: 194 - DNA, 23 bp, Homo sapiens
  source 1..23: mol_type = genomic DNA
ataatcacggggtgggggggggt

SEQ ID NO: 195 - DNA, 21 bp, Homo sapiens
  source 1..21: mol_type = genomic DNA
gctgataatcacggggtgggg

SEQ ID NO: 196 - DNA, 21 bp, Homo sapiens
  source 1..21: mol_type = genomic DNA
gataatcacggggtggggggg

SEQ ID NO: 197 - DNA, 21 bp, Homo sapiens
  source 1..21: mol_type = genomic DNA
aatcacggggtgggggggggt

SEQ ID NO: 198 - DNA, 23 bp, Homo sapiens
  source 1..23: mol_type = genomic DNA
acggggtgggggggggttctcat

SEQ ID NO: 199 - DNA, 23 bp, Homo sapiens
  source 1..23: mol_type = genomic DNA
gggtgggggggggttctcatagt

SEQ ID NO: 200 - DNA, 24 bp, Homo sapiens
  source 1..24: mol_type = genomic DNA
cttttcttgattatgagacttaaa

SEQ ID NO: 201 - DNA, 22 bp, Homo sapiens
  source 1..22: mol_type = genomic DNA
cttgattatgagacttaaacgg

SEQ ID NO: 202 - DNA, 22 bp, Homo sapiens
  source 1..22: mol_type = genomic DNA
gattatgagacttaaacggaaa

SEQ ID NO: 203 - DNA, 23 bp, Homo sapiens
  source 1..23: mol_type = genomic DNA
tgattatgagacttaaacggaaa

SEQ ID NO: 204 - DNA, 21 bp, Homo sapiens
  source 1..21: mol_type = genomic DNA
attatgagacttaaacggaaa

SEQ ID NO: 205 - DNA, 25 bp, Homo sapiens
  source 1..25: mol_type = genomic DNA
aacggaaattttgaaattttgggtt

SEQ ID NO: 206 - DNA, 24 bp, Homo sapiens
  source 1..24: mol_type = genomic DNA
acggaaattttgaaattttgggtt

SEQ ID NO: 207 - DNA, 23 bp, Homo sapiens
  source 1..23: mol_type = genomic DNA
ctttgccctttacgagtcatctg

SEQ ID NO: 208 - DNA, 22 bp, Homo sapiens
  source 1..22: mol_type = genomic DNA
gccctttacgagtcatctgaaa

SEQ ID NO: 209 - DNA, 22 bp, Homo sapiens
  source 1..22: mol_type = genomic DNA
ctttacgagtcatctgaaaata

SEQ ID NO: 210 - DNA, 23 bp, Homo sapiens
  source 1..23: mol_type = genomic DNA
cctttacgagtcatctgaaaata

SEQ ID NO: 211 - DNA, 21 bp, Homo sapiens
  source 1..21: mol_type = genomic DNA
acgagtcatctgaaaatatga

SEQ ID NO: 212 - DNA, 25 bp, Homo sapiens
  source 1..25: mol_type = genomic DNA
tgaaaatatgatttcttcccctcac

SEQ ID NO: 213 - DNA, 25 bp, Homo sapiens
  source 1..25: mol_type = genomic DNA
aaatatgatttcttcccctcaccac

SEQ ID NO: 214 - DNA, 24 bp, Homo sapiens
  source 1..24: mol_type = genomic DNA
gaaaatatgatttcttcccctcac

SEQ ID NO: 215 - DNA, 24 bp, Homo sapiens
  source 1..24: mol_type = genomic DNA
aatatgatttcttcccctcaccac

SEQ ID NO: 216 - DNA, 24 bp, Homo sapiens
  source 1..24: mol_type = genomic DNA
atgatttcttcccctcaccacaga

SEQ ID NO: 217 - DNA, 24 bp, Homo sapiens
  source 1..24: mol_type = genomic DNA
atttcttcccctcaccacagaggt

SEQ ID NO: 218 - DNA, 25 bp, Homo sapiens
  source 1..25: mol_type = genomic DNA
gatttcttcccctcaccacagaggt

SEQ ID NO: 219 - DNA, 23 bp, Homo sapiens
  source 1..23: mol_type = genomic DNA
cttcccctcaccacagaggtgag

SEQ ID NO: 220 - DNA, 23 bp, Homo sapiens
  source 1..23: mol_type = genomic DNA
cccctcaccacagaggtgagagg

SEQ ID NO: 221 - DNA, 22 bp, Homo sapiens
  source 1..22: mol_type = genomic DNA
ccctcaccacagaggtgagagg

SEQ ID NO: 222 - DNA, 21 bp, Homo sapiens
  source 1..21: mol_type = genomic DNA
cctcaccacagaggtgagagg

SEQ ID NO: 223 - DNA, 23 bp, Homo sapiens
  source 1..23: mol_type = genomic DNA
accacagaggtgagaggtatcaa

SEQ ID NO: 224 - DNA, 23 bp, Homo sapiens
  source 1..23: mol_type = genomic DNA
acagaggtgagaggtatcaatga

SEQ ID NO: 225 - DNA, 23 bp, Homo sapiens
  source 1..23: mol_type = genomic DNA
gaggtgagaggtatcaatgagat

SEQ ID NO: 226 - DNA, 23 bp, Homo sapiens
  source 1..23: mol_type = genomic DNA
gtgagaggtatcaatgagataat

SEQ ID NO: 227 - DNA, 23 bp, Homo sapiens
  source 1..23: mol_type = genomic DNA
agaggtatcaatgagataatagg

SEQ ID NO: 228 - DNA, 23 bp, Homo sapiens
  source 1..23: mol_type = genomic DNA
ggtatcaatgagataatagggct

SEQ ID NO: 229 - DNA, 22 bp, Homo sapiens
  source 1..22: mol_type = genomic DNA
ccacagaggtgagaggtatcaa

SEQ ID NO: 230 - DNA, 22 bp, Homo sapiens
  source 1..22: mol_type = genomic DNA
cagaggtgagaggtatcaatga

SEQ ID NO: 231 - DNA, 22 bp, Homo sapiens
  source 1..22: mol_type = genomic DNA
aggtgagaggtatcaatgagat

SEQ ID NO: 232 - DNA, 22 bp, Homo sapiens
  source 1..22: mol_type = genomic DNA
tgagaggtatcaatgagataat

SEQ ID NO: 233 - DNA, 22 bp, Homo sapiens
  source 1..22: mol_type = genomic DNA
gaggtatcaatgagataatagg

SEQ ID NO: 234 - DNA, 22 bp, Homo sapiens
  source 1..22: mol_type = genomic DNA
gtatcaatgagataatagggct

SEQ ID NO: 235 - DNA, 21 bp, Homo sapiens
  source 1..21: mol_type = genomic DNA
caatgagataatagggctcat

SEQ ID NO: 236 - DNA, 21 bp, Homo sapiens
  source 1..21: mol_type = genomic DNA
tgagataatagggctcatgag

SEQ ID NO: 237 - DNA, 21 bp, Homo sapiens
  source 1..21: mol_type = genomic DNA
gataatagggctcatgagaaa

SEQ ID NO: 238 - DNA, 21 bp, Homo sapiens
  source 1..21: mol_type = genomic DNA
aatagggctcatgagaaacca

SEQ ID NO: 239 - DNA, 21 bp, Homo sapiens
  source 1..21: mol_type = genomic DNA
agggctcatgagaaaccacag

SEQ ID NO: 240 - DNA, 23 bp, Homo sapiens
  source 1..23: mol_type = genomic DNA
aatgagataatagggctcatgag

SEQ ID NO: 241 - DNA, 23 bp, Homo sapiens
  source 1..23: mol_type = genomic DNA
gagataatagggctcatgagaaa

SEQ ID NO: 242 - DNA, 23 bp, Homo sapiens
  source 1..23: mol_type = genomic DNA
ataatagggctcatgagaaacca

SEQ ID NO: 243 - DNA, 23 bp, Homo sapiens
  source 1..23: mol_type = genomic DNA
atagggctcatgagaaaccacag

SEQ ID NO: 244 - DNA, 22 bp, Homo sapiens
  source 1..22: mol_type = genomic DNA
atgagataatagggctcatgag

SEQ ID NO: 245 - DNA, 22 bp, Homo sapiens
  source 1..22: mol_type = genomic DNA
agataatagggctcatgagaaa

SEQ ID NO: 246 - DNA, 22 bp, Homo sapiens
  source 1..22: mol_type = genomic DNA
tagggctcatgagaaaccacag

SEQ ID NO: 247 - DNA, 24 bp, Homo sapiens
  source 1..24: mol_type = genomic DNA
aaaacaaaagtgtatagagtttga

SEQ ID NO: 248 - DNA, 24 bp, Homo sapiens
  source 1..24: mol_type = genomic DNA
acaaaagtgtatagagtttgaaaa

SEQ ID NO: 249 - DNA, 24 bp, Homo sapiens
  source 1..24: mol_type = genomic DNA
aaagtgtatagagtttgaaaaaaa

SEQ ID NO: 250 - DNA, 24 bp, Homo sapiens
  source 1..24: mol_type = genomic DNA
gtgtatagagtttgaaaaaaaaaa

SEQ ID NO: 251 - DNA, 23 bp, Homo sapiens
  source 1..23: mol_type = genomic DNA
aaacaaaagtgtatagagtttga

SEQ ID NO: 252 - DNA, 23 bp, Homo sapiens
  source 1..23: mol_type = genomic DNA
caaaagtgtatagagtttgaaaa

SEQ ID NO: 253 - DNA, 23 bp, Homo sapiens
  source 1..23: mol_type = genomic DNA
aagtgtatagagtttgaaaaaaa

SEQ ID NO: 254 - DNA, 22 bp, Homo sapiens
  source 1..22: mol_type = genomic DNA
aacaaaagtgtatagagtttga

SEQ ID NO: 255 - DNA, 22 bp, Homo sapiens
  source 1..22: mol_type = genomic DNA
aaaagtgtatagagtttgaaaa

SEQ ID NO: 256 - DNA, 22 bp, Homo sapiens
  source 1..22: mol_type = genomic DNA
agtgtatagagtttgaaaaaaa

SEQ ID NO: 257 - DNA, 22 bp, Homo sapiens
  source 1..22: mol_type = genomic DNA
gtatagagtttgaaaaaaaaaa

SEQ ID NO: 258 - DNA, 22 bp, Homo sapiens
  source 1..22: mol_type = genomic DNA
tagagtttgaaaaaaaaaaaac

SEQ ID NO: 259 - DNA, 22 bp, Homo sapiens
  source 1..22: mol_type = genomic DNA
agtttgaaaaaaaaaaaacaag

SEQ ID NO: 260 - DNA, 23 bp, Homo sapiens
  source 1..23: mol_type = genomic DNA
atagagtttgaaaaaaaaaaaac

SEQ ID NO: 261 - DNA, 23 bp, Homo sapiens
  source 1..23: mol_type = genomic DNA
gagtttgaaaaaaaaaaaacaag

SEQ ID NO: 262 - DNA, 21 bp, Homo sapiens
  source 1..21: mol_type = genomic DNA
agagtttgaaaaaaaaaaaac

SEQ ID NO: 263 - DNA, 21 bp, Homo sapiens
  source 1..21: mol_type = genomic DNA
gtttgaaaaaaaaaaaacaag

SEQ ID NO: 264 - DNA, 21 bp, Homo sapiens
  source 1..21: mol_type = genomic DNA
tgaaaaaaaaaaaacaaggga

SEQ ID NO: 265 - DNA, 21 bp, Homo sapiens
  source 1..21: mol_type = genomic DNA
aaaaaaaaaaacaagggaaaa

SEQ ID NO: 266 - DNA, 21 bp, Homo sapiens
  source 1..21: mol_type = genomic DNA
aaaaaaaacaagggaaaagaa

SEQ ID NO: 267 - DNA, 21 bp, Homo sapiens
  source 1..21: mol_type = genomic DNA
aaaaacaagggaaaagaacta

SEQ ID NO: 268 - DNA, 21 bp, Homo sapiens
  source 1..21: mol_type = genomic DNA
aacaagggaaaagaactaaaa

SEQ ID NO: 269 - DNA, 22 bp, Homo sapiens
  source 1..22: mol_type = genomic DNA
aagggaaaagaactaaaataaa

SEQ ID NO: 270 - DNA, 23 bp, Homo sapiens
  source 1..23: mol_type = genomic DNA
ggaaaagaactaaaataaatcac

SEQ ID NO: 271 - DNA, 23 bp, Homo sapiens
  source 1..23: mol_type = genomic DNA
aaagaactaaaataaatcacagg

SEQ ID NO: 272 - DNA, 23 bp, Homo sapiens
  source 1..23: mol_type = genomic DNA
gaactaaaataaatcacagggcc

SEQ ID NO: 273 - DNA, 23 bp, Homo sapiens
  source 1..23: mol_type = genomic DNA
ctaaaataaatcacagggccaac

SEQ ID NO: 274 - DNA, 23 bp, Homo sapiens
  source 1..23: mol_type = genomic DNA
aaataaatcacagggccaacccg

SEQ ID NO: 275 - DNA, 21 bp, Homo sapiens
  source 1..21: mol_type = genomic DNA
gaaaaaaaaaaaacaagggaa

SEQ ID NO: 276 - DNA, 21 bp, Homo sapiens
  source 1..21: mol_type = genomic DNA
aaaaaaaaaacaagggaaaag

SEQ ID NO: 277 - DNA, 21 bp, Homo sapiens
  source 1..21: mol_type = genomic DNA
aaaaaaacaagggaaaagaac

SEQ ID NO: 278 - DNA, 22 bp, Homo sapiens
  source 1..22: mol_type = genomic DNA
aaaacaagggaaaagaactaaa

SEQ ID NO: 279 - DNA, 22 bp, Homo sapiens
  source 1..22: mol_type = genomic DNA
acaagggaaaagaactaaaata

SEQ ID NO: 280 - DNA, 22 bp, Homo sapiens
  source 1..22: mol_type = genomic DNA
agggaaaagaactaaaataaat

SEQ ID NO: 281 - DNA, 22 bp, Homo sapiens
  source 1..22: mol_type = genomic DNA
gaaaagaactaaaataaatcac

SEQ ID NO: 282 - DNA, 22 bp, Homo sapiens
  source 1..22: mol_type = genomic DNA
aagaactaaaataaatcacagg

SEQ ID NO: 283 - DNA, 22 bp, Homo sapiens
  source 1..22: mol_type = genomic DNA
aactaaaataaatcacagggcc

SEQ ID NO: 284 - DNA, 21 bp, Homo sapiens
  source 1..21: mol_type = genomic DNA
aaaaaaaaaaaacaagggaaa

SEQ ID NO: 285 - DNA, 21 bp, Homo sapiens
  source 1..21: mol_type = genomic DNA
aaaaaaaaacaagggaaaaga

SEQ ID NO: 286 - DNA, 21 bp, Homo sapiens
  source 1..21: mol_type = genomic DNA
aaaaaacaagggaaaagaact

SEQ ID NO: 287 - DNA, 21 bp, Homo sapiens
  source 1..21: mol_type = genomic DNA
aaacaagggaaaagaactaaa

SEQ ID NO: 288 - DNA, 21 bp, Homo sapiens
  source 1..21: mol_type = genomic DNA
caagggaaaagaactaaaata

SEQ ID NO: 289 - DNA, 21 bp, Homo sapiens
  source 1..21: mol_type = genomic DNA
gggaaaagaactaaaataaat

SEQ ID NO: 290 - DNA, 21 bp, Homo sapiens
  source 1..21: mol_type = genomic DNA
aaaagaactaaaataaatcac

SEQ ID NO: 291 - DNA, 21 bp, Homo sapiens
  source 1..21: mol_type = genomic DNA
agaactaaaataaatcacagg

SEQ ID NO: 292 - DNA, 21 bp, Homo sapiens
  source 1..21: mol_type = genomic DNA
actaaaataaatcacagggcc

SEQ ID NO: 293 - DNA, 21 bp, Homo sapiens
  source 1..21: mol_type = genomic DNA
aaaataaatcacagggccaac

SEQ ID NO: 294 - DNA, 21 bp, Homo sapiens
  source 1..21: mol_type = genomic DNA
ataaatcacagggccaacccg

SEQ ID NO: 295 - DNA, 21 bp, Homo sapiens
  source 1..21: mol_type = genomic DNA
aatcacagggccaacccgagg

SEQ ID NO: 296 - DNA, 21 bp, Homo sapiens
  source 1..21: mol_type = genomic DNA
cacagggccaacccgaggcag

SEQ ID NO: 297 - DNA, 21 bp, Homo sapiens
  source 1..21: mol_type = genomic DNA
agggccaacccgaggcaggca

SEQ ID NO: 298 - DNA, 21 bp, Homo sapiens
  source 1..21: mol_type = genomic DNA
gccaacccgaggcaggcagag

SEQ ID NO: 299 - DNA, 21 bp, Homo sapiens
  source 1..21: mol_type = genomic DNA
aacccgaggcaggcagagaca

SEQ ID NO: 300 - DNA, 21 bp, Homo sapiens
  source 1..21: mol_type = genomic DNA
ccgaggcaggcagagacacca

SEQ ID NO: 301 - DNA, 23 bp, Homo sapiens
  source 1..23: mol_type = genomic DNA
aggcaggcagagacaccattctg

SEQ ID NO: 302 - DNA, 23 bp, Homo sapiens
  source 1..23: mol_type = genomic DNA
caggcagagacaccattctgtga

SEQ ID NO: 303 - DNA, 23 bp, Homo sapiens
  source 1..23: mol_type = genomic DNA
gcagagacaccattctgtgagtg

SEQ ID NO: 304 - DNA, 23 bp, Homo sapiens
  source 1..23: mol_type = genomic DNA
gagacaccattctgtgagtgaga

SEQ ID NO: 305 - DNA, 23 bp, Homo sapiens
  source 1..23: mol_type = genomic DNA
acaccattctgtgagtgagagga

SEQ ID NO: 306 - DNA, 24 bp, Homo sapiens
  source 1..24: mol_type = genomic DNA
ccattctgtgagtgagaggatatt

SEQ ID NO: 307 - DNA, 22 bp, Homo sapiens
  source 1..22: mol_type = genomic DNA
aataaatcacagggccaacccg

SEQ ID NO: 308 - DNA, 22 bp, Homo sapiens
  source 1..22: mol_type = genomic DNA
aaatcacagggccaacccgagg

SEQ ID NO: 309 - DNA, 21 bp, Homo sapiens
  source 1..21: mol_type = genomic DNA
acagggccaacccgaggcagg

SEQ ID NO: 310 - DNA, 21 bp, Homo sapiens
  source 1..21: mol_type = genomic DNA
gggccaacccgaggcaggcag

SEQ ID NO: 311 - DNA, 21 bp, Homo sapiens
  source 1..21: mol_type = genomic DNA
ccaacccgaggcaggcagaga

SEQ ID NO: 312 - DNA, 21 bp, Homo sapiens
  source 1..21: mol_type = genomic DNA
acccgaggcaggcagagacac

SEQ ID NO: 313 - DNA, 21 bp, Homo sapiens
  source 1..21: mol_type = genomic DNA
cgaggcaggcagagacaccat

SEQ ID NO: 314 - DNA, 22 bp, Homo sapiens
  source 1..22: mol_type = genomic DNA
ggcaggcagagacaccattctg

SEQ ID NO: 315 - DNA, 22 bp, Homo sapiens
  source 1..22: mol_type = genomic DNA
aggcagagacaccattctgtga

SEQ ID NO: 316 - DNA, 22 bp, Homo sapiens
  source 1..22: mol_type = genomic DNA
cagagacaccattctgtgagtg

SEQ ID NO: 317 - DNA, 22 bp, Homo sapiens
  source 1..22: mol_type = genomic DNA
agacaccattctgtgagtgaga

SEQ ID NO: 318 - DNA, 22 bp, Homo sapiens
  source 1..22: mol_type = genomic DNA
caccattctgtgagtgagagga

SEQ ID NO: 319 - DNA, 23 bp, Homo sapiens
  source 1..23: mol_type = genomic DNA
cattctgtgagtgagaggatatt

SEQ ID NO: 320 - DNA, 21 bp, Homo sapiens
  source 1..21: mol_type = genomic DNA
cagggccaacccgaggcaggc

SEQ ID NO: 321 - DNA, 21 bp, Homo sapiens
  source 1..21: mol_type = genomic DNA
ggccaacccgaggcaggcaga

SEQ ID NO: 322 - DNA, 21 bp, Homo sapiens
  source 1..21: mol_type = genomic DNA
caacccgaggcaggcagagac

SEQ ID NO: 323 - DNA, 21 bp, Homo sapiens
  source 1..21: mol_type = genomic DNA
cccgaggcaggcagagacacc

SEQ ID NO: 324 - DNA, 21 bp, Homo sapiens
  source 1..21: mol_type = genomic DNA
gaggcaggcagagacaccatt

SEQ ID NO: 325 - DNA, 21 bp, Homo sapiens
  source 1..21: mol_type = genomic DNA
gcaggcagagacaccattctg

SEQ ID NO: 326 - DNA, 21 bp, Homo sapiens
  source 1..21: mol_type = genomic DNA
ggcagagacaccattctgtga

SEQ ID NO: 327 - DNA, 21 bp, Homo sapiens
  source 1..21: mol_type = genomic DNA
agagacaccattctgtgagtg

SEQ ID NO: 328 - DNA, 21 bp, Homo sapiens
  source 1..21: mol_type = genomic DNA
gacaccattctgtgagtgaga

SEQ ID NO: 329 - DNA, 21 bp, Homo sapiens
  source 1..21: mol_type = genomic DNA
accattctgtgagtgagagga

SEQ ID NO: 330 - DNA, 22 bp, Homo sapiens
  source 1..22: mol_type = genomic DNA
attctgtgagtgagaggatatt

SEQ ID NO: 331 - DNA, 22 bp, Homo sapiens
  source 1..22: mol_type = genomic DNA
ctgtgagtgagaggatatttga

SEQ ID NO: 332 - DNA, 22 bp, Homo sapiens
  source 1..22: mol_type = genomic DNA
tgagtgagaggatatttgaggg

SEQ ID NO: 333 - DNA, 23 bp, Homo sapiens
  source 1..23: mol_type = genomic DNA
gtgagtgagaggatatttgaggg

SEQ ID NO: 334 - DNA, 21 bp, Homo sapiens
  source 1..21: mol_type = genomic DNA
gagtgagaggatatttgaggg

SEQ ID NO: 335 - DNA, 23 bp, Homo sapiens
  source 1..23: mol_type = genomic DNA
gagggtctctggggaaagaaaga

SEQ ID NO: 336 - DNA, 23 bp, Homo sapiens
  source 1..23: mol_type = genomic DNA
ggtctctggggaaagaaagagaa

SEQ ID NO: 337 - DNA, 22 bp, Homo sapiens
  source 1..22: mol_type = genomic DNA
agggtctctggggaaagaaaga

SEQ ID NO: 338 - DNA, 22 bp, Homo sapiens
  source 1..22: mol_type = genomic DNA
gtctctggggaaagaaagagaa

SEQ ID NO: 339 - DNA, 22 bp, Homo sapiens
  source 1..22: mol_type = genomic DNA
ctggggaaagaaagagaatctg

SEQ ID NO: 340 - DNA, 22 bp, Homo sapiens
  source 1..22: mol_type = genomic DNA
gggaaagaaagagaatctgaag

SEQ ID NO: 341 - DNA, 24 bp, Homo sapiens
  source 1..24: mol_type = genomic DNA
aaagaaagagaatctgaagctcta

SEQ ID NO: 342 - DNA, 25 bp, Homo sapiens
  source 1..25: mol_type = genomic DNA
gaaagagaatctgaagctctatgtg

SEQ ID NO: 343 - DNA, 25 bp, Homo sapiens
  source 1..25: mol_type = genomic DNA
agagaatctgaagctctatgtgtgg

SEQ ID NO: 344 - DNA, 25 bp, Homo sapiens
  source 1..25: mol_type = genomic DNA
gaatctgaagctctatgtgtggatg

SEQ ID NO: 345 - DNA, 21 bp, Homo sapiens
  source 1..21: mol_type = genomic DNA
tggggaaagaaagagaatctg

SEQ ID NO: 346 - DNA, 21 bp, Homo sapiens
  source 1..21: mol_type = genomic DNA
ggaaagaaagagaatctgaag

SEQ ID NO: 347 - DNA, 23 bp, Homo sapiens
  source 1..23: mol_type = genomic DNA
aagaaagagaatctgaagctcta

SEQ ID NO: 348 - DNA, 24 bp, Homo sapiens
  source 1..24: mol_type = genomic DNA
aaagagaatctgaagctctatgtg

SEQ ID NO: 349 - DNA, 24 bp, Homo sapiens
  source 1..24: mol_type = genomic DNA
gagaatctgaagctctatgtgtgg

SEQ ID NO: 350 - DNA, 24 bp, Homo sapiens
  source 1..24: mol_type = genomic DNA
aatctgaagctctatgtgtggatg

SEQ ID NO: 351 - DNA, 24 bp, Homo sapiens
  source 1..24: mol_type = genomic DNA
ctgaagctctatgtgtggatggga

SEQ ID NO: 352 - DNA, 24 bp, Homo sapiens
  source 1..24: mol_type = genomic DNA
aagctctatgtgtggatgggaaat

SEQ ID NO: 353 - DNA, 23 bp, Homo sapiens
  source 1..23: mol_type = genomic DNA
ggggaaagaaagagaatctgaag

SEQ ID NO: 354 - DNA, 25 bp, Homo sapiens
  source 1..25: mol_type = genomic DNA
gaaagaaagagaatctgaagctcta

SEQ ID NO: 355 - DNA, 26 bp, Homo sapiens
  source 1..26: mol_type = genomic DNA
agaaagagaatctgaagctctatgtg

SEQ ID NO: 356 - DNA, 26 bp, Homo sapiens
  source 1..26: mol_type = genomic DNA
aagagaatctgaagctctatgtgtgg

SEQ ID NO: 357 - DNA, 26 bp, Homo sapiens
  source 1..26: mol_type = genomic DNA
agaatctgaagctctatgtgtggatg

SEQ ID NO: 358 - DNA, 23 bp, Homo sapiens
  source 1..23: mol_type = genomic DNA
tgaagctctatgtgtggatggga

SEQ ID NO: 359 - DNA, 23 bp, Homo sapiens
  source 1..23: mol_type = genomic DNA
agctctatgtgtggatgggaaat

SEQ ID NO: 360 - DNA, 22 bp, Homo sapiens
  source 1..22: mol_type = genomic DNA
gaagctctatgtgtggatggga

SEQ ID NO: 361 - DNA, 22 bp, Homo sapiens
  source 1..22: mol_type = genomic DNA
gctctatgtgtggatgggaaat

SEQ ID NO: 362 - DNA, 22 bp, Homo sapiens
  source 1..22: mol_type = genomic DNA
ctatgtgtggatgggaaatgcc

SEQ ID NO: 363 - DNA, 23 bp, Homo sapiens
  source 1..23: mol_type = genomic DNA
atgtgtggatgggaaatgccagg

SEQ ID NO: 364 - DNA, 24 bp, Homo sapiens
  source 1..24: mol_type = genomic DNA
atttcccatccacacatagagctt

SEQ ID NO: 365 - DNA, 23 bp, Homo sapiens
  source 1..23: mol_type = genomic DNA
cccatccacacatagagcttcag

SEQ ID NO: 366 - DNA, 22 bp, Homo sapiens
  source 1..22: mol_type = genomic DNA
ccatccacacatagagcttcag

SEQ ID NO: 367 - DNA, 21 bp, Homo sapiens
  source 1..21: mol_type = genomic DNA
catccacacatagagcttcag

SEQ ID NO: 368 - DNA, 21 bp, Homo sapiens
  source 1..21: mol_type = genomic DNA
ccacacatagagcttcagatt

SEQ ID NO: 369 - DNA, 23 bp, Homo sapiens
  source 1..23: mol_type = genomic DNA
cacatagagcttcagattctctt

SEQ ID NO: 370 - DNA, 24 bp, Homo sapiens
  source 1..24: mol_type = genomic DNA
atagagcttcagattctctttctt

SEQ ID NO: 371 - DNA, 25 bp, Homo sapiens
  source 1..25: mol_type = genomic DNA
gagcttcagattctctttctttccc

SEQ ID NO: 372 - DNA, 25 bp, Homo sapiens
  source 1..25: mol_type = genomic DNA
cttcagattctctttctttccccag

SEQ ID NO: 373 - DNA, 25 bp, Homo sapiens
  source 1..25: mol_type = genomic DNA
cagattctctttctttccccagaga

SEQ ID NO: 374 - DNA, 25 bp, Homo sapiens
  source 1..25: mol_type = genomic DNA
attctctttctttccccagagaccc

SEQ ID NO: 375 - DNA, 25 bp, Homo sapiens
  source 1..25: mol_type = genomic DNA
cacacatagagcttcagattctctt

SEQ ID NO: 376 - DNA, 26 bp, Homo sapiens
  source 1..26: mol_type = genomic DNA
acatagagcttcagattctctttctt

SEQ ID NO: 377 - DNA, 27 bp, Homo sapiens
  source 1..27: mol_type = genomic DNA
tagagcttcagattctctttctttccc

SEQ ID NO: 378 - DNA, 27 bp, Homo sapiens
  source 1..27: mol_type = genomic DNA
agcttcagattctctttctttccccag

SEQ ID NO: 379 - DNA, 24 bp, Homo sapiens
  source 1..24: mol_type = genomic DNA
acacatagagcttcagattctctt

SEQ ID NO: 380 - DNA, 25 bp, Homo sapiens
  source 1..25: mol_type = genomic DNA
catagagcttcagattctctttctt

SEQ ID NO: 381 - DNA, 26 bp, Homo sapiens
  source 1..26: mol_type = genomic DNA
agagcttcagattctctttctttccc

SEQ ID NO: 382 - DNA, 26 bp, Homo sapiens
  source 1..26: mol_type = genomic DNA
gcttcagattctctttctttccccag

SEQ ID NO: 383 - DNA, 24 bp, Homo sapiens
  source 1..24: mol_type = genomic DNA
ctttctttccccagagaccctcaa

SEQ ID NO: 384 - DNA, 23 bp, Homo sapiens
  source 1..23: mol_type = genomic DNA
ctttccccagagaccctcaaata

SEQ ID NO: 385 - DNA, 23 bp, Homo sapiens
  source 1..23: mol_type = genomic DNA
ccccagagaccctcaaatatcct

SEQ ID NO: 386 - DNA, 25 bp, Homo sapiens
  source 1..25: mol_type = genomic DNA
cagagaccctcaaatatcctctcac

SEQ ID NO: 387 - DNA, 26 bp, Homo sapiens
  source 1..26: mol_type = genomic DNA
agaccctcaaatatcctctcactcac

SEQ ID NO: 388 - DNA, 26 bp, Homo sapiens
  source 1..26: mol_type = genomic DNA
ccctcaaatatcctctcactcacaga

SEQ ID NO: 389 - DNA, 22 bp, Homo sapiens
  source 1..22: mol_type = genomic DNA
cccagagaccctcaaatatcct

SEQ ID NO: 390 - DNA, 24 bp, Homo sapiens
  source 1..24: mol_type = genomic DNA
agagaccctcaaatatcctctcac

SEQ ID NO: 391 - DNA, 25 bp, Homo sapiens
  source 1..25: mol_type = genomic DNA
gaccctcaaatatcctctcactcac

SEQ ID NO: 392 - DNA, 25 bp, Homo sapiens
  source 1..25: mol_type = genomic DNA
cctcaaatatcctctcactcacaga

SEQ ID NO: 393 - DNA, 25 bp, Homo sapiens
  source 1..25: mol_type = genomic DNA
caaatatcctctcactcacagaatg

SEQ ID NO: 394 - DNA, 25 bp, Homo sapiens
  source 1..25: mol_type = genomic DNA
atatcctctcactcacagaatggtg

SEQ ID NO: 395 - DNA, 26 bp, Homo sapiens
  source 1..26: mol_type = genomic DNA
ccagagaccctcaaatatcctctcac

SEQ ID NO: 396 - DNA, 27 bp, Homo sapiens
  source 1..27: mol_type = genomic DNA
gagaccctcaaatatcctctcactcac

SEQ ID NO: 397 - DNA, 27 bp, Homo sapiens
  source 1..27: mol_type = genomic DNA
accctcaaatatcctctcactcacaga

SEQ ID NO: 398 - DNA, 26 bp, Homo sapiens
  source 1..26: mol_type = genomic DNA
aatatcctctcactcacagaatggtg

SEQ ID NO: 399 - DNA, 23 bp, Homo sapiens
  source 1..23: mol_type = genomic DNA
acagaatggtgtctctgcctgcc

SEQ ID NO: 400 - DNA, 24 bp, Homo sapiens
  source 1..24: mol_type = genomic DNA
gaatggtgtctctgcctgcctcgg

SEQ ID NO: 401 - DNA, 24 bp, Homo sapiens
  source 1..24: mol_type = genomic DNA
tggtgtctctgcctgcctcgggtt

SEQ ID NO: 402 - DNA, 22 bp, Homo sapiens
  source 1..22: mol_type = genomic DNA
cagaatggtgtctctgcctgcc

SEQ ID NO: 403 - DNA, 23 bp, Homo sapiens
  source 1..23: mol_type = genomic DNA
aatggtgtctctgcctgcctcgg

SEQ ID NO: 404 - DNA, 23 bp, Homo sapiens
  source 1..23: mol_type = genomic DNA
ggtgtctctgcctgcctcgggtt

SEQ ID NO: 405 - DNA, 23 bp, Homo sapiens
  source 1..23: mol_type = genomic DNA
gtctctgcctgcctcgggttggc

SEQ ID NO: 406 - DNA, 22 bp, Homo sapiens
  source 1..22: mol_type = genomic DNA
ctgcctgcctcgggttggccct

SEQ ID NO: 407 - DNA, 22 bp, Homo sapiens
  source 1..22: mol_type = genomic DNA
cctgcctcgggttggccctgtg

SEQ ID NO: 408 - DNA, 22 bp, Homo sapiens
  source 1..22: mol_type = genomic DNA
gcctcgggttggccctgtgatt

SEQ ID NO: 409 - DNA, 23 bp, Homo sapiens
  source 1..23: mol_type = genomic DNA
gcctgcctcgggttggccctgtg

SEQ ID NO: 410 - DNA, 21 bp, Homo sapiens
  source 1..21: mol_type = genomic DNA
ctgcctcgggttggccctgtg

SEQ ID NO: 411 - DNA, 21 bp, Homo sapiens
  source 1..21: mol_type = genomic DNA
cctcgggttggccctgtgatt

SEQ ID NO: 412 - DNA, 22 bp, Homo sapiens
  source 1..22: mol_type = genomic DNA
cgggttggccctgtgatttatt

SEQ ID NO: 413 - DNA, 23 bp, Homo sapiens
  source 1..23: mol_type = genomic DNA
gttggccctgtgatttattttag

SEQ ID NO: 414 - DNA, 25 bp, Homo sapiens
  source 1..25: mol_type = genomic DNA
ggccctgtgatttattttagttctt

SEQ ID NO: 415 - DNA, 27 bp, Homo sapiens
  source 1..27: mol_type = genomic DNA
cctgtgatttattttagttcttttccc

SEQ ID NO: 416 - DNA, 29 bp, Homo sapiens
  source 1..29: mol_type = genomic DNA
gtgatttattttagttcttttcccttgtt

SEQ ID NO: 417 - DNA, 25 bp, Homo sapiens
  source 1..25: mol_type = genomic DNA
gggttggccctgtgatttattttag

SEQ ID NO: 418 - DNA, 24 bp, Homo sapiens
  source 1..24: mol_type = genomic DNA
ggttggccctgtgatttattttag

SEQ ID NO: 419 - DNA, 26 bp, Homo sapiens
  source 1..26: mol_type = genomic DNA
tggccctgtgatttattttagttctt

SEQ ID NO: 420 - DNA, 28 bp, Homo sapiens
  source 1..28: mol_type = genomic DNA
ccctgtgatttattttagttcttttccc

SEQ ID NO: 421 - DNA, 26 bp, Homo sapiens
  source 1..26: mol_type = genomic DNA
caaactctatacacttttgttttaaa

SEQ ID NO: 422 - DNA, 26 bp, Homo sapiens
  source 1..26: mol_type = genomic DNA
actctatacacttttgttttaaaaac

SEQ ID NO: 423 - DNA, 27 bp, Homo sapiens
  source 1..27: mol_type = genomic DNA
ctatacacttttgttttaaaaactgtg

SEQ ID NO: 424 - DNA, 28 bp, Homo sapiens
  source 1..28: mol_type = genomic DNA
aaactctatacacttttgttttaaaaac

SEQ ID NO: 425 - DNA, 27 bp, Homo sapiens
  source 1..27: mol_type = genomic DNA
aactctatacacttttgttttaaaaac

SEQ ID NO: 426 - DNA, 26 bp, Homo sapiens
  source 1..26: mol_type = genomic DNA
acacttttgttttaaaaactgtggtt

SEQ ID NO: 427 - DNA, 25 bp, Homo sapiens
  source 1..25: mol_type = genomic DNA
atacctctcacctctgtggtgaggg

SEQ ID NO: 428 - DNA, 25 bp, Homo sapiens
  source 1..25: mol_type = genomic DNA
cctctcacctctgtggtgaggggaa

SEQ ID NO: 429 - DNA, 23 bp, Homo sapiens
  source 1..23: mol_type = genomic DNA
cacctctgtggtgaggggaagaa

SEQ ID NO: 430 - DNA, 22 bp, Homo sapiens
  source 1..22: mol_type = genomic DNA
acctctgtggtgaggggaagaa

SEQ ID NO: 431 - DNA, 21 bp, Homo sapiens
  source 1..21: mol_type = genomic DNA
cctctgtggtgaggggaagaa

SEQ ID NO: 432 - DNA, 23 bp, Homo sapiens
  source 1..23: mol_type = genomic DNA
ctgtggtgaggggaagaaatcat

SEQ ID NO: 433 - DNA, 23 bp, Homo sapiens
  source 1..23: mol_type = genomic DNA
tggtgaggggaagaaatcatatt

SEQ ID NO: 434 - DNA, 25 bp, Homo sapiens
  source 1..25: mol_type = genomic DNA
tgaggggaagaaatcatattttcag

SEQ ID NO: 435 - DNA, 25 bp, Homo sapiens
  source 1..25: mol_type = genomic DNA
ggggaagaaatcatattttcagatg

SEQ ID NO: 436 - DNA, 25 bp, Homo sapiens
  source 1..25: mol_type = genomic DNA
gaagaaatcatattttcagatgact

SEQ ID NO: 437 - DNA, 25 bp, Homo sapiens
  source 1..25: mol_type = genomic DNA
gaaatcatattttcagatgactcgt

SEQ ID NO: 438 - DNA, 21 bp, Homo sapiens
  source 1..21: mol_type = genomic DNA
gtggtgaggggaagaaatcat

SEQ ID NO: 439 - DNA, 21 bp, Homo sapiens
  source 1..21: mol_type = genomic DNA
gtgaggggaagaaatcatatt

SEQ ID NO: 440 - DNA, 23 bp, Homo sapiens
  source 1..23: mol_type = genomic DNA
aggggaagaaatcatattttcag

SEQ ID NO: 441 - DNA, 23 bp, Homo sapiens
  source 1..23: mol_type = genomic DNA
ggaagaaatcatattttcagatg

SEQ ID NO: 442 - DNA, 23 bp, Homo sapiens
  source 1..23: mol_type = genomic DNA
agaaatcatattttcagatgact

SEQ ID NO: 443 - DNA, 23 bp, Homo sapiens
  source 1..23: mol_type = genomic DNA
aatcatattttcagatgactcgt

SEQ ID NO: 444 - DNA, 23 bp, Homo sapiens
  source 1..23: mol_type = genomic DNA
catattttcagatgactcgtaaa

SEQ ID NO: 445 - DNA, 23 bp, Homo sapiens
  source 1..23: mol_type = genomic DNA
attttcagatgactcgtaaaggg

SEQ ID NO: 446 - DNA, 22 bp, Homo sapiens
  source 1..22: mol_type = genomic DNA
ggtgaggggaagaaatcatatt

SEQ ID NO: 447 - DNA, 24 bp, Homo sapiens
  source 1..24: mol_type = genomic DNA
gaggggaagaaatcatattttcag

SEQ ID NO: 448 - DNA, 24 bp, Homo sapiens
  source 1..24: mol_type = genomic DNA
gggaagaaatcatattttcagatg

SEQ ID NO: 449 - DNA, 24 bp, Homo sapiens
  source 1..24: mol_type = genomic DNA
aagaaatcatattttcagatgact

SEQ ID NO: 450 - DNA, 24 bp, Homo sapiens
  source 1..24: mol_type = genomic DNA
aaatcatattttcagatgactcgt

SEQ ID NO: 451 - DNA, 21 bp, Homo sapiens
  source 1..21: mol_type = genomic DNA
cagatgactcgtaaagggcaa

SEQ ID NO: 452 - DNA, 21 bp, Homo sapiens
  source 1..21: mol_type = genomic DNA
atgactcgtaaagggcaaaga

SEQ ID NO: 453 - DNA, 21 bp, Homo sapiens
  source 1..21: mol_type = genomic DNA
actcgtaaagggcaaagaaaa

SEQ ID NO: 454 - DNA, 21 bp, Homo sapiens
  source 1..21: mol_type = genomic DNA
cgtaaagggcaaagaaaaaaa

SEQ ID NO: 455 - DNA, 21 bp, Homo sapiens
  source 1..21: mol_type = genomic DNA
aaagggcaaagaaaaaaaccc

SEQ ID NO: 456 - DNA, 21 bp, Homo sapiens
  source 1..21: mol_type = genomic DNA
gggcaaagaaaaaaacccaaa

SEQ ID NO: 457 - DNA, 21 bp, Homo sapiens
  source 1..21: mol_type = genomic DNA
caaagaaaaaaacccaaaatt

SEQ ID NO: 458 - DNA, 22 bp, Homo sapiens
  source 1..22: mol_type = genomic DNA
agaaaaaaacccaaaatttcaa

SEQ ID NO: 459 - DNA, 22 bp, Homo sapiens
  source 1..22: mol_type = genomic DNA
aaaaaacccaaaatttcaaaat

SEQ ID NO: 460 - DNA, 24 bp, Homo sapiens
  source 1..24: mol_type = genomic DNA
aaacccaaaatttcaaaatttccg

SEQ ID NO: 461 - DNA, 23 bp, Homo sapiens
  source 1..23: mol_type = genomic DNA
agatgactcgtaaagggcaaaga

SEQ ID NO: 462 - DNA, 23 bp, Homo sapiens
  source 1..23: mol_type = genomic DNA
tgactcgtaaagggcaaagaaaa

SEQ ID NO: 463 - DNA, 22 bp, Homo sapiens
  source 1..22: mol_type = genomic DNA
gatgactcgtaaagggcaaaga

SEQ ID NO: 464 - DNA, 22 bp, Homo sapiens
  source 1..22: mol_type = genomic DNA
gactcgtaaagggcaaagaaaa

SEQ ID NO: 465 - DNA, 21 bp, Homo sapiens
  source 1..21: mol_type = genomic DNA
gtaaagggcaaagaaaaaaac

SEQ ID NO: 466 - DNA, 21 bp, Homo sapiens
  source 1..21: mol_type = genomic DNA
aagggcaaagaaaaaaaccca

SEQ ID NO: 467 - DNA, 21 bp, Homo sapiens
  source 1..21: mol_type = genomic DNA
ggcaaagaaaaaaacccaaaa

SEQ ID NO: 468 - DNA, 21 bp, Homo sapiens
  source 1..21: mol_type = genomic DNA
agggcaaagaaaaaaacccaa

SEQ ID NO: 469 - DNA, 21 bp, Homo sapiens
  source 1..21: mol_type = genomic DNA
gcaaagaaaaaaacccaaaat

SEQ ID NO: 470 - DNA, 23 bp, Homo sapiens
  source 1..23: mol_type = genomic DNA
aagaaaaaaacccaaaatttcaa

SEQ ID NO: 471 - DNA, 23 bp, Homo sapiens
  source 1..23: mol_type = genomic DNA
aaaaaaacccaaaatttcaaaat

SEQ ID NO: 472 - DNA, 25 bp, Homo sapiens
  source 1..25: mol_type = genomic DNA
aaaacccaaaatttcaaaatttccg

SEQ ID NO: 473 - DNA, 23 bp, Homo sapiens
  source 1..23: mol_type = genomic DNA
agtctcataatcaagaaaaggag

SEQ ID NO: 474 - DNA, 22 bp, Homo sapiens
  source 1..22: mol_type = genomic DNA
gtctcataatcaagaaaaggag

SEQ ID NO: 475 - DNA, 21 bp, Homo sapiens
  source 1..21: mol_type = genomic DNA
cataatcaagaaaaggagaaa

SEQ ID NO: 476 - DNA, 21 bp, Homo sapiens
  source 1..21: mol_type = genomic DNA
aatcaagaaaaggagaaacac

SEQ ID NO: 477 - DNA, 21 bp, Homo sapiens
  source 1..21: mol_type = genomic DNA
caagaaaaggagaaacacaga

SEQ ID NO: 478 - DNA, 21 bp, Homo sapiens
  source 1..21: mol_type = genomic DNA
gaaaaggagaaacacagagag

SEQ ID NO: 479 - DNA, 21 bp, Homo sapiens
  source 1..21: mol_type = genomic DNA
aaggagaaacacagagagaga

SEQ ID NO: 480 - DNA, 21 bp, Homo sapiens
  source 1..21: mol_type = genomic DNA
gagaaacacagagagagagaa

SEQ ID NO: 481 - DNA, 21 bp, Homo sapiens
  source 1..21: mol_type = genomic DNA
aaacacagagagagagaaaaa

SEQ ID NO: 482 - DNA, 21 bp, Homo sapiens
  source 1..21: mol_type = genomic DNA
cacagagagagagaaaaaaaa

SEQ ID NO: 483 - DNA, 21 bp, Homo sapiens
  source 1..21: mol_type = genomic DNA
agagagagagaaaaaaaaaac

SEQ ID NO: 484 - DNA, 22 bp, Homo sapiens
  source 1..22: mol_type = genomic DNA
gagagagaaaaaaaaaactatg

SEQ ID NO: 485 - DNA, 22 bp, Homo sapiens
  source 1..22: mol_type = genomic DNA
agagaaaaaaaaaactatgaga

SEQ ID NO: 486 - DNA, 22 bp, Homo sapiens
  source 1..22: mol_type = genomic DNA
gaaaaaaaaaactatgagaacc

SEQ ID NO: 487 - DNA, 22 bp, Homo sapiens
  source 1..22: mol_type = genomic DNA
aaaaaaaactatgagaaccccc

SEQ ID NO: 488 - DNA, 22 bp, Homo sapiens
  source 1..22: mol_type = genomic DNA
aaaaactatgagaacccccccc

SEQ ID NO: 489 - DNA, 22 bp, Homo sapiens
  source 1..22: mol_type = genomic DNA
aactatgagaacccccccccac

SEQ ID NO: 490 - DNA, 23 bp, Homo sapiens
  source 1..23: mol_type = genomic DNA
ataatcaagaaaaggagaaacac

SEQ ID NO: 491 - DNA, 21 bp, Homo sapiens
  source 1..21: mol_type = genomic DNA
aagaaaaggagaaacacagag

SEQ ID NO: 492 - DNA, 21 bp, Homo sapiens
  source 1..21: mol_type = genomic DNA
aaaaggagaaacacagagaga

SEQ ID NO: 493 - DNA, 21 bp, Homo sapiens
  source 1..21: mol_type = genomic DNA
aggagaaacacagagagagag

SEQ ID NO: 494 - DNA, 21 bp, Homo sapiens
  source 1..21: mol_type = genomic DNA
agaaacacagagagagagaaa

SEQ ID NO: 495 - DNA, 21 bp, Homo sapiens
  source 1..21: mol_type = genomic DNA
aacacagagagagagaaaaaa

SEQ ID NO: 496 - DNA, 21 bp, Homo sapiens
  source 1..21: mol_type = genomic DNA
acagagagagagaaaaaaaaa

SEQ ID NO: 497 - DNA, 21 bp, Homo sapiens
  source 1..21: mol_type = genomic DNA
gagagagagaaaaaaaaaact

SEQ ID NO: 498 - DNA, 21 bp, Homo sapiens
  source 1..21: mol_type = genomic DNA
agagagaaaaaaaaaactatg

SEQ ID NO: 499 - DNA, 21 bp, Homo sapiens
  source 1..21: mol_type = genomic DNA
gagaaaaaaaaaactatgaga

SEQ ID NO: 500 - DNA, 21 bp, Homo sapiens
  source 1..21: mol_type = genomic DNA
aaaaaaaaaactatgagaacc

SEQ ID NO: 501 - DNA, 21 bp, Homo sapiens
  source 1..21: mol_type = genomic DNA
aaaaaaactatgagaaccccc

SEQ ID NO: 502 - DNA, 21 bp, Homo sapiens
  source 1..21: mol_type = genomic DNA
aaaactatgagaacccccccc

SEQ ID NO: 503 - DNA, 21 bp, Homo sapiens
  source 1..21: mol_type = genomic DNA
actatgagaacccccccccac

SEQ ID NO: 504 - DNA, 21 bp, Homo sapiens
  source 1..21: mol_type = genomic DNA
atgagaacccccccccacccc

SEQ ID NO: 505 - DNA, 21 bp, Homo sapiens
  source 1..21: mol_type = genomic DNA
agaacccccccccaccccgtg

SEQ ID NO: 506 - DNA, 21 bp, Homo sapiens
  source 1..21: mol_type = genomic DNA
acccccccccaccccgtgatt

SEQ ID NO: 507 - DNA, 23 bp, Homo sapiens
  source 1..23: mol_type = genomic DNA
cccccccaccccgtgattatcag

SEQ ID NO: 508 - DNA, 23 bp, Homo sapiens
  source 1..23: mol_type = genomic DNA
ccccaccccgtgattatcagcgc

SEQ ID NO: 509 - DNA, 23 bp, Homo sapiens
  source 1..23: mol_type = genomic DNA
caccccgtgattatcagcgcaca

SEQ ID NO: 510 - DNA, 23 bp, Homo sapiens
  source 1..23: mol_type = genomic DNA
cccgtgattatcagcgcacacac

SEQ ID NO: 511 - DNA, 24 bp, Homo sapiens
  source 1..24: mol_type = genomic DNA
gtgattatcagcgcacacactcat

SEQ ID NO: 512 - DNA, 24 bp, Homo sapiens
  source 1..24: mol_type = genomic DNA
attatcagcgcacacactcatcga

SEQ ID NO: 513 - DNA, 21 bp, Homo sapiens
  source 1..21: mol_type = genomic DNA
agaaaaggagaaacacagaga

SEQ ID NO: 514 - DNA, 21 bp, Homo sapiens
  source 1..21: mol_type = genomic DNA
aaaggagaaacacagagagag

SEQ ID NO: 515 - DNA, 21 bp, Homo sapiens
  source 1..21: mol_type = genomic DNA
ggagaaacacagagagagaga

SEQ ID NO: 516 - DNA, 21 bp, Homo sapiens
  source 1..21: mol_type = genomic DNA
gaaacacagagagagagaaaa

SEQ ID NO: 517 - DNA, 21 bp, Homo sapiens
  source 1..21: mol_type = genomic DNA
acacagagagagagaaaaaaa

SEQ ID NO: 518 - DNA, 21 bp, Homo sapiens
  source 1..21: mol_type = genomic DNA
cagagagagagaaaaaaaaaa

SEQ ID NO: 519 - DNA, 21 bp, Homo sapiens
  source 1..21: mol_type = genomic DNA
agagagagaaaaaaaaaacta

SEQ ID NO: 520 - DNA, 21 bp, Homo sapiens
  source 1..21: mol_type = genomic DNA
gagagaaaaaaaaaactatga

SEQ ID NO: 521 - DNA, 21 bp, Homo sapiens
  source 1..21: mol_type = genomic DNA
agaaaaaaaaaactatgagaa

SEQ ID NO: 522 - DNA, 21 bp, Homo sapiens
  source 1..21: mol_type = genomic DNA
aaaaaaaaactatgagaaccc

SEQ ID NO: 523 - DNA, 21 bp, Homo sapiens
  source 1..21: mol_type = genomic DNA
aaaaaactatgagaacccccc

SEQ ID NO: 524 - DNA, 21 bp, Homo sapiens
  source 1..21: mol_type = genomic DNA
aaactatgagaaccccccccc

SEQ ID NO: 525 - DNA, 21 bp, Homo sapiens
  source 1..21: mol_type = genomic DNA
ctatgagaacccccccccacc

SEQ ID NO: 526 - DNA, 21 bp, Homo sapiens
  source 1..21: mol_type = genomic DNA
tgagaacccccccccaccccg

SEQ ID NO: 527 - DNA, 21 bp, Homo sapiens
  source 1..21: mol_type = genomic DNA
gaacccccccccaccccgtga

SEQ ID NO: 528 - DNA, 21 bp, Homo sapiens
  source 1..21: mol_type = genomic DNA
gagaacccccccccaccccgt

SEQ ID NO: 529 - DNA, 21 bp, Homo sapiens
  source 1..21: mol_type = genomic DNA
aacccccccccaccccgtgat

SEQ ID NO: 530 - DNA, 22 bp, Homo sapiens
  source 1..22: mol_type = genomic DNA
cagcgcacacactcatcgaaaa

SEQ ID NO: 531 - DNA, 22 bp, Homo sapiens
  source 1..22: mol_type = genomic DNA
cgcacacactcatcgaaaaaaa

SEQ ID NO: 532 - DNA, 24 bp, Homo sapiens
  source 1..24: mol_type = genomic DNA
acacactcatcgaaaaaaatttgg

SEQ ID NO: 533 - DNA, 24 bp, Homo sapiens
  source 1..24: mol_type = genomic DNA
cactcatcgaaaaaaatttggatt

SEQ ID NO: 534 - DNA, 21 bp, Homo sapiens
  source 1..21: mol_type = genomic DNA
agcgcacacactcatcgaaaa

SEQ ID NO: 535 - DNA, 21 bp, Homo sapiens
  source 1..21: mol_type = genomic DNA
gcacacactcatcgaaaaaaa

SEQ ID NO: 536 - DNA, 23 bp, Homo sapiens
  source 1..23: mol_type = genomic DNA
cacactcatcgaaaaaaatttgg

SEQ ID NO: 537 - DNA, 23 bp, Homo sapiens
  source 1..23: mol_type = genomic DNA
actcatcgaaaaaaatttggatt

SEQ ID NO: 538 - DNA, 23 bp, Homo sapiens
  source 1..23: mol_type = genomic DNA
catcgaaaaaaatttggattatt

SEQ ID NO: 539 - DNA, 23 bp, Homo sapiens
  source 1..23: mol_type = genomic DNA
cgaaaaaaatttggattattaga

SEQ ID NO: 540 - DNA, 23 bp, Homo sapiens
  source 1..23: mol_type = genomic DNA
aaaaaatttggattattagaaga

SEQ ID NO: 541 - DNA, 23 bp, Homo sapiens
  source 1..23: mol_type = genomic DNA
aaatttggattattagaagagag

SEQ ID NO: 542 - DNA, 23 bp, Homo sapiens
  source 1..23: mol_type = genomic DNA
gcgcacacactcatcgaaaaaaa

SEQ ID NO: 543 - DNA, 25 bp, Homo sapiens
  source 1..25: mol_type = genomic DNA
cacacactcatcgaaaaaaatttgg

SEQ ID NO: 544 - DNA, 25 bp, Homo sapiens
  source 1..25: mol_type = genomic DNA
acactcatcgaaaaaaatttggatt

SEQ ID NO: 545 - DNA, 22 bp, Homo sapiens
  source 1..22: mol_type = genomic DNA
gaaaaaaatttggattattaga

SEQ ID NO: 546 - DNA, 22 bp, Homo sapiens
  source 1..22: mol_type = genomic DNA
aaaaatttggattattagaaga

SEQ ID NO: 547 - DNA, 22 bp, Homo sapiens
  source 1..22: mol_type = genomic DNA
aatttggattattagaagagag

SEQ ID NO: 548 - DNA, 21 bp, Homo sapiens
  source 1..21: mol_type = genomic DNA
aaaaaaatttggattattaga

SEQ ID NO: 549 - DNA, 21 bp, Homo sapiens
  source 1..21: mol_type = genomic DNA
aaaatttggattattagaaga

SEQ ID NO: 550 - DNA, 21 bp, Homo sapiens
  source 1..21: mol_type = genomic DNA
atttggattattagaagagag

SEQ ID NO: 551 - DNA, 21 bp, Homo sapiens
  source 1..21: mol_type = genomic DNA
tggattattagaagagagagg

SEQ ID NO: 552 - DNA, 22 bp, Homo sapiens
  source 1..22: mol_type = genomic DNA
attattagaagagagaggtctg

SEQ ID NO: 553 - DNA, 22 bp, Homo sapiens
  source 1..22: mol_type = genomic DNA
attagaagagagaggtctgcgg

SEQ ID NO: 554 - DNA, 22 bp, Homo sapiens
  source 1..22: mol_type = genomic DNA
agaagagagaggtctgcggctt

SEQ ID NO: 555 - DNA, 22 bp, Homo sapiens
  source 1..22: mol_type = genomic DNA
agagagaggtctgcggcttcca

SEQ ID NO: 556 - DNA, 22 bp, Homo sapiens
  source 1..22: mol_type = genomic DNA
gagaggtctgcggcttccacac

SEQ ID NO: 557 - DNA, 22 bp, Homo sapiens
  source 1..22: mol_type = genomic DNA
aggtctgcggcttccacaccgt

SEQ ID NO: 558 - DNA, 24 bp, Homo sapiens
  source 1..24: mol_type = genomic DNA
ggattattagaagagagaggtctg

SEQ ID NO: 559 - DNA, 23 bp, Homo sapiens
  source 1..23: mol_type = genomic DNA
gattattagaagagagaggtctg

SEQ ID NO: 560 - DNA, 22 bp, Homo sapiens
  source 1..22: mol_type = genomic DNA
tagaagagagaggtctgcggct

SEQ ID NO: 561 - DNA, 23 bp, Homo sapiens
  source 1..23: mol_type = genomic DNA
aagagagaggtctgcggcttcca

SEQ ID NO: 562 - DNA, 23 bp, Homo sapiens
  source 1..23: mol_type = genomic DNA
agagaggtctgcggcttccacac

SEQ ID NO: 563 - DNA, 23 bp, Homo sapiens
  source 1..23: mol_type = genomic DNA
gaggtctgcggcttccacaccgt

SEQ ID NO: 564 - DNA, 23 bp, Homo sapiens
  source 1..23: mol_type = genomic DNA
gtctgcggcttccacaccgtaca

SEQ ID NO: 565 - DNA, 21 bp, Homo sapiens
  source 1..21: mol_type = genomic DNA
gaagagagaggtctgcggctt

SEQ ID NO: 566 - DNA, 21 bp, Homo sapiens
  source 1..21: mol_type = genomic DNA
gagagaggtctgcggcttcca

SEQ ID NO: 567 - DNA, 21 bp, Homo sapiens
  source 1..21: mol_type = genomic DNA
agaggtctgcggcttccacac

SEQ ID NO: 568 - DNA, 21 bp, Homo sapiens
  source 1..21: mol_type = genomic DNA
ggtctgcggcttccacaccgt

SEQ ID NO: 569 - DNA, 21 bp, Homo sapiens
  source 1..21: mol_type = genomic DNA
ctgcggcttccacaccgtaca

SEQ ID NO: 570 - DNA, 21 bp, Homo sapiens
  source 1..21: mol_type = genomic DNA
cggcttccacaccgtacagcg

SEQ ID NO: 571 - DNA, 21 bp, Homo sapiens
  source 1..21: mol_type = genomic DNA
cttccacaccgtacagcgtgg

SEQ ID NO: 572 - DNA, 22 bp, Homo sapiens
  source 1..22: mol_type = genomic DNA
gcggcttccacaccgtacagcg

SEQ ID NO: 573 - DNA, 22 bp, Homo sapiens
  source 1..22: mol_type = genomic DNA
gcttccacaccgtacagcgtgg

SEQ ID NO: 574 - DNA, 23 bp, Homo sapiens
  source 1..23: mol_type = genomic DNA
ggcttccacaccgtacagcgtgg

SEQ ID NO: 575 - DNA, 26 bp, Homo sapiens
  source 1..26: mol_type = genomic DNA
cttctcggtataaaagcaaagttgtt

SEQ ID NO: 576 - DNA, 26 bp, Homo sapiens
  source 1..26: mol_type = genomic DNA
cggtataaaagcaaagttgtttttga

SEQ ID NO: 577 - DNA, 25 bp, Homo sapiens
  source 1..25: mol_type = genomic DNA
ggtataaaagcaaagttgtttttga

SEQ ID NO: 578 - DNA, 26 bp, Homo sapiens
  source 1..26: mol_type = genomic DNA
ataaaagcaaagttgtttttgatacg

SEQ ID NO: 579 - DNA, 26 bp, Homo sapiens
  source 1..26: mol_type = genomic DNA
aaagcaaagttgtttttgatacgtga

SEQ ID NO: 580 - DNA, 26 bp, Homo sapiens
  source 1..26: mol_type = genomic DNA
gcaaagttgtttttgatacgtgacag

SEQ ID NO: 581 - DNA, 24 bp, Homo sapiens
  source 1..24: mol_type = genomic DNA
gtataaaagcaaagttgtttttga

SEQ ID NO: 582 - DNA, 24 bp, Homo sapiens
  source 1..24: mol_type = genomic DNA
aaaagcaaagttgtttttgatacg

SEQ ID NO: 583 - DNA, 24 bp, Homo sapiens
  source 1..24: mol_type = genomic DNA
agcaaagttgtttttgatacgtga

SEQ ID NO: 584 - DNA, 24 bp, Homo sapiens
  source 1..24: mol_type = genomic DNA
aaagttgtttttgatacgtgacag

SEQ ID NO: 585 - DNA, 25 bp, Homo sapiens
  source 1..25: mol_type = genomic DNA
aagcaaagttgtttttgatacgtga

SEQ ID NO: 586 - DNA, 25 bp, Homo sapiens
  source 1..25: mol_type = genomic DNA
caaagttgtttttgatacgtgacag

SEQ ID NO: 587 - DNA, 23 bp, Homo sapiens
  source 1..23: mol_type = genomic DNA
atacgtgacagtttcccacaagc

SEQ ID NO: 588 - DNA, 23 bp, Homo sapiens
  source 1..23: mol_type = genomic DNA
cgtgacagtttcccacaagccag

SEQ ID NO: 589 - DNA, 23 bp, Homo sapiens
  source 1..23: mol_type = genomic DNA
gacagtttcccacaagccaggct

SEQ ID NO: 590 - DNA, 23 bp, Homo sapiens
  source 1..23: mol_type = genomic DNA
agtttcccacaagccaggctgat

SEQ ID NO: 591 - DNA, 22 bp, Homo sapiens
  source 1..22: mol_type = genomic DNA
gtgacagtttcccacaagccag

SEQ ID NO: 592 - DNA, 22 bp, Homo sapiens
  source 1..22: mol_type = genomic DNA
acagtttcccacaagccaggct

SEQ ID NO: 593 - DNA, 22 bp, Homo sapiens
  source 1..22: mol_type = genomic DNA
gtttcccacaagccaggctgat

SEQ ID NO: 594 - DNA, 21 bp, Homo sapiens
  source 1..21: mol_type = genomic DNA
cccacaagccaggctgatcct

SEQ ID NO: 595 - DNA, 25 bp, Homo sapiens
  source 1..25: mol_type = genomic DNA
cttttctgtcagtccacttcaccaa

SEQ ID NO: 596 - DNA, 23 bp, Homo sapiens
  source 1..23: mol_type = genomic DNA
ctgtcagtccacttcaccaaggt

SEQ ID NO: 597 - DNA, 21 bp, Homo sapiens
  source 1..21: mol_type = genomic DNA
gtcagtccacttcaccaaggt

SEQ ID NO: 598 - DNA, 21 bp, Homo sapiens
  source 1..21: mol_type = genomic DNA
agtccacttcaccaaggtgag

SEQ ID NO: 599 - DNA, 22 bp, Homo sapiens
  source 1..22: mol_type = genomic DNA
ccacttcaccaaggtgagtgtc

SEQ ID NO: 600 - DNA, 22 bp, Homo sapiens
  source 1..22: mol_type = genomic DNA
cttcaccaaggtgagtgtccct

SEQ ID NO: 601 - DNA, 22 bp, Homo sapiens
  source 1..22: mol_type = genomic DNA
caccaaggtgagtgtccctgct

SEQ ID NO: 602 - DNA, 24 bp, Homo sapiens
  source 1..24: mol_type = genomic DNA
caaggtgagtgtccctgctctccc

SEQ ID NO: 603 - DNA, 24 bp, Homo sapiens
  source 1..24: mol_type = genomic DNA
ggtgagtgtccctgctctccccta

SEQ ID NO: 604 - DNA, 24 bp, Homo sapiens
  source 1..24: mol_type = genomic DNA
gagtgtccctgctctcccctacca

SEQ ID NO: 605 - DNA, 22 bp, Homo sapiens
  source 1..22: mol_type = genomic DNA
cagtccacttcaccaaggtgag

SEQ ID NO: 606 - DNA, 24 bp, Homo sapiens
  source 1..24: mol_type = genomic DNA
gtccacttcaccaaggtgagtgtc

SEQ ID NO: 607 - DNA, 24 bp, Homo sapiens
  source 1..24: mol_type = genomic DNA
cacttcaccaaggtgagtgtccct

SEQ ID NO: 608 - DNA, 23 bp, Homo sapiens
  source 1..23: mol_type = genomic DNA
acttcaccaaggtgagtgtccct

SEQ ID NO: 609 - protein, 211 aa, synthetic construct
  REGION 1..211: note = source = /note="Description of Artificial Sequence: Syntheticpolypeptide"
LEPGEKPYKCPECGKSFSDPGNLVRHQRTHTGEKPYKCPECGKSFSRSDELVRHQRTHTGEKPYKCPECGKSFSQSSNLVRHQRTHTGEKPYKCPECGKSFSTSGHLVRHQRTHTGEKPYKCPECGKSFSTTGALTEHQRTHTGEKPYKCPECGKSFSSKKALTEHQRTHTHPRAPIPKPFQYKCPECGKSFSSKKALTEHQRTHTGKKTS

SEQ ID NO: 610 - protein, 211 aa, synthetic construct
  REGION 1..211: note = source = /note="Description of Artificial Sequence: Syntheticpolypeptide"
LEPGEKPYKCPECGKSFSSPADLTRHQRTHTGEKPYKCPECGKSFSRNDALTEHQRTHTGEKPYKCPECGKSFSQRAHLERHQRTHTGEKPYKCPECGKSFSHRTTLTNHQRTHTGEKPYKCPECGKSFSQAGHLASHQRTHTGEKPYKCPECGKSFSRSDHLTTHQRTHTHPRAPIPKPFQYKCPECGKSFSDKKDLTRHQRTHTGKKTS

SEQ ID NO: 611 - protein, 204 aa, synthetic construct
  REGION 1..204: note = source = /note="Description of Artificial Sequence: Syntheticpolypeptide"
LEPGEKPYKCPECGKSFSSPADLTRHQRTHTGEKPYKCPECGKSFSRNDALTEHQRTHTGEKPYKCPECGKSFSQRAHLERHQRTHTGEKPYKCPECGKSFSHRTTLTNHQRTHTGEKPYKCPECGKSFSQAGHLASHQRTHTGEKPYKCPECGKSFSRSDHLTTHQRTHTGEKPYKCPECGKSFSTKNSLTEHQRTHTGKKTS

SEQ ID NO: 612 - protein, 204 aa, synthetic construct
  REGION 1..204: note = source = /note="Description of Artificial Sequence: Syntheticpolypeptide"
LEPGEKPYKCPECGKSFSQSSNLVRHQRTHTGEKPYKCPECGKSFSSPADLTRHQRTHTGEKPYKCPECGKSFSRNDALTEHQRTHTGEKPYKCPECGKSFSQRAHLERHQRTHTGEKPYKCPECGKSFSHRTTLTNHQRTHTGEKPYKCPECGKSFSQAGHLASHQRTHTGEKPYKCPECGKSFSRSDHLTTHQRTHTGKKTS

SEQ ID NO: 613 - protein, 204 aa, synthetic construct
  REGION 1..204: note = source = /note="Description of Artificial Sequence: Syntheticpolypeptide"
LEPGEKPYKCPECGKSFSRKDNLKNHQRTHTGEKPYKCPECGKSFSQSSNLVRHQRTHTGEKPYKCPECGKSFSSPADLTRHQRTHTGEKPYKCPECGKSFSRNDALTEHQRTHTGEKPYKCPECGKSFSQRAHLERHQRTHTGEKPYKCPECGKSFSHRTTLTNHQRTHTGEKPYKCPECGKSFSQAGHLASHQRTHTGKKTS

SEQ ID NO: 614 - protein, 204 aa, synthetic construct
  REGION 1..204: note = source = /note="Description of Artificial Sequence: Syntheticpolypeptide"
LEPGEKPYKCPECGKSFSTSGNLVRHQRTHTGEKPYKCPECGKSFSRKDNLKNHQRTHTGEKPYKCPECGKSFSQSSNLVRHQRTHTGEKPYKCPECGKSFSSPADLTRHQRTHTGEKPYKCPECGKSFSRNDALTEHQRTHTGEKPYKCPECGKSFSQRAHLERHQRTHTGEKPYKCPECGKSFSHRTTLTNHQRTHTGKKTS

SEQ ID NO: 615 - protein, 204 aa, synthetic construct
  REGION 1..204: note = source = /note="Description of Artificial Sequence: Syntheticpolypeptide"
LEPGEKPYKCPECGKSFSRADNLTEHQRTHTGEKPYKCPECGKSFSTSGNLVRHQRTHTGEKPYKCPECGKSFSRKDNLKNHQRTHTGEKPYKCPECGKSFSQSSNLVRHQRTHTGEKPYKCPECGKSFSSPADLTRHQRTHTGEKPYKCPECGKSFSRNDALTEHQRTHTGEKPYKCPECGKSFSQRAHLERHQRTHTGKKTS

SEQ ID NO: 616 - protein, 204 aa, synthetic construct
  REGION 1..204: note = source = /note="Description of Artificial Sequence: Syntheticpolypeptide"
LEPGEKPYKCPECGKSFSTKNSLTEHQRTHTGEKPYKCPECGKSFSRADNLTEHQRTHTGEKPYKCPECGKSFSTSGNLVRHQRTHTGEKPYKCPECGKSFSRKDNLKNHQRTHTGEKPYKCPECGKSFSQSSNLVRHQRTHTGEKPYKCPECGKSFSSPADLTRHQRTHTGEKPYKCPECGKSFSRNDALTEHQRTHTGKKTS

SEQ ID NO: 617 - protein, 204 aa, synthetic construct
  REGION 1..204: note = source = /note="Description of Artificial Sequence: Syntheticpolypeptide"
LEPGEKPYKCPECGKSFSDPGHLVRHQRTHTGEKPYKCPECGKSFSTKNSLTEHQRTHTGEKPYKCPECGKSFSRADNLTEHQRTHTGEKPYKCPECGKSFSTSGNLVRHQRTHTGEKPYKCPECGKSFSRKDNLKNHQRTHTGEKPYKCPECGKSFSQSSNLVRHQRTHTGEKPYKCPECGKSFSSPADLTRHQRTHTGKKTS

SEQ ID NO: 618 - protein, 215 aa, synthetic construct
  REGION 1..215: note = source = /note="Description of Artificial Sequence: Syntheticpolypeptide"
LEPGEKPYKCPECGKSFSRSDELVRHQRTHTPNPHRRTDPSHKPFQYKCPECGKSFSDPGHLVRHQRTHTGEKPYKCPECGKSFSTKNSLTEHQRTHTGEKPYKCPECGKSFSRADNLTEHQRTHTGEKPYKCPECGKSFSTSGNLVRHQRTHTGEKPYKCPECGKSFSRKDNLKNHQRTHTGEKPYKCPECGKSFSQSSNLVRHQRTHTGKKTS

SEQ ID NO: 619 - protein, 215 aa, synthetic construct
  REGION 1..215: note = source = /note="Description of Artificial Sequence: Syntheticpolypeptide"
LEPGEKPYKCPECGKSFSQRAHLERHQRTHTGEKPYKCPECGKSFSRSDELVRHQRTHTPNPHRRTDPSHKPFQYKCPECGKSFSDPGHLVRHQRTHTGEKPYKCPECGKSFSTKNSLTEHQRTHTGEKPYKCPECGKSFSRADNLTEHQRTHTGEKPYKCPECGKSFSTSGNLVRHQRTHTGEKPYKCPECGKSFSRKDNLKNHQRTHTGKKTS

SEQ ID NO: 620 - protein, 215 aa, synthetic construct
  REGION 1..215: note = source = /note="Description of Artificial Sequence: Syntheticpolypeptide"
LEPGEKPYKCPECGKSFSDSGNLRVHQRTHTGEKPYKCPECGKSFSQRAHLERHQRTHTGEKPYKCPECGKSFSRSDELVRHQRTHTPNPHRRTDPSHKPFQYKCPECGKSFSDPGHLVRHQRTHTGEKPYKCPECGKSFSTKNSLTEHQRTHTGEKPYKCPECGKSFSRADNLTEHQRTHTGEKPYKCPECGKSFSTSGNLVRHQRTHTGKKTS

SEQ ID NO: 621 - protein, 222 aa, synthetic construct
  REGION 1..222: note = source = /note="Description of Artificial Sequence: Syntheticpolypeptide"
LEPGEKPYKCPECGKSFSDPGALVRHQRTHTHPRAPIPKPFQYKCPECGKSFSDSGNLRVHQRTHTGEKPYKCPECGKSFSQRAHLERHQRTHTGEKPYKCPECGKSFSRSDELVRHQRTHTPNPHRRTDPSHKPFQYKCPECGKSFSDPGHLVRHQRTHTGEKPYKCPECGKSFSTKNSLTEHQRTHTGEKPYKCPECGKSFSRADNLTEHQRTHTGKKTS

SEQ ID NO: 622 - protein, 222 aa, synthetic construct
  REGION 1..222: note = source = /note="Description of Artificial Sequence: Syntheticpolypeptide"
LEPGEKPYKCPECGKSFSRTDTLRDHQRTHTGEKPYKCPECGKSFSDPGALVRHQRTHTHPRAPIPKPFQYKCPECGKSFSDSGNLRVHQRTHTGEKPYKCPECGKSFSQRAHLERHQRTHTGEKPYKCPECGKSFSRSDELVRHQRTHTPNPHRRTDPSHKPFQYKCPECGKSFSDPGHLVRHQRTHTGEKPYKCPECGKSFSTKNSLTEHQRTHTGKKTS

SEQ ID NO: 623 - protein, 204 aa, synthetic construct
  REGION 1..204: note = source = /note="Description of Artificial Sequence: Syntheticpolypeptide"
LEPGEKPYKCPECGKSFSQRANLRAHQRTHTGEKPYKCPECGKSFSQLAHLRAHQRTHTGEKPYKCPECGKSFSDPGNLVRHQRTHTGEKPYKCPECGKSFSTHLDLIRHQRTHTGEKPYKCPECGKSFSRSDHLTTHQRTHTGEKPYKCPECGKSFSRKDNLKNHQRTHTGEKPYKCPECGKSFSRSDELVRHQRTHTGKKTS

SEQ ID NO: 624 - protein, 204 aa, synthetic construct
  REGION 1..204: note = source = /note="Description of Artificial Sequence: Syntheticpolypeptide"
LEPGEKPYKCPECGKSFSQRAHLERHQRTHTGEKPYKCPECGKSFSQRANLRAHQRTHTGEKPYKCPECGKSFSQLAHLRAHQRTHTGEKPYKCPECGKSFSDPGNLVRHQRTHTGEKPYKCPECGKSFSTHLDLIRHQRTHTGEKPYKCPECGKSFSRSDHLTTHQRTHTGEKPYKCPECGKSFSRKDNLKNHQRTHTGKKTS

SEQ ID NO: 625 - protein, 211 aa, synthetic construct
  REGION 1..211: note = source = /note="Description of Artificial Sequence: Syntheticpolypeptide"
LEPGEKPYKCPECGKSFSRADNLTEHQRTHTHPRAPIPKPFQYKCPECGKSFSQRAHLERHQRTHTGEKPYKCPECGKSFSQRANLRAHQRTHTGEKPYKCPECGKSFSQLAHLRAHQRTHTGEKPYKCPECGKSFSDPGNLVRHQRTHTGEKPYKCPECGKSFSTHLDLIRHQRTHTGEKPYKCPECGKSFSRSDHLTTHQRTHTGKKTS

SEQ ID NO: 626 - protein, 211 aa, synthetic construct
  REGION 1..211: note = source = /note="Description of Artificial Sequence: Syntheticpolypeptide"
LEPGEKPYKCPECGKSFSTKNSLTEHQRTHTGEKPYKCPECGKSFSRADNLTEHQRTHTHPRAPIPKPFQYKCPECGKSFSQRAHLERHQRTHTGEKPYKCPECGKSFSQRANLRAHQRTHTGEKPYKCPECGKSFSQLAHLRAHQRTHTGEKPYKCPECGKSFSDPGNLVRHQRTHTGEKPYKCPECGKSFSTHLDLIRHQRTHTGKKTS

SEQ ID NO: 627 - protein, 211 aa, synthetic construct
  REGION 1..211: note = source = /note="Description of Artificial Sequence: Syntheticpolypeptide"
LEPGEKPYKCPECGKSFSDPGHLVRHQRTHTGEKPYKCPECGKSFSTKNSLTEHQRTHTGEKPYKCPECGKSFSRADNLTEHQRTHTHPRAPIPKPFQYKCPECGKSFSQRAHLERHQRTHTGEKPYKCPECGKSFSQRANLRAHQRTHTGEKPYKCPECGKSFSQLAHLRAHQRTHTGEKPYKCPECGKSFSDPGNLVRHQRTHTGKKTS

SEQ ID NO: 628 - protein, 222 aa, synthetic construct
  REGION 1..222: note = source = /note="Description of Artificial Sequence: Syntheticpolypeptide"
LEPGEKPYKCPECGKSFSRSDELVRHQRTHTPNPHRRTDPSHKPFQYKCPECGKSFSDPGHLVRHQRTHTGEKPYKCPECGKSFSTKNSLTEHQRTHTGEKPYKCPECGKSFSRADNLTEHQRTHTHPRAPIPKPFQYKCPECGKSFSQRAHLERHQRTHTGEKPYKCPECGKSFSQRANLRAHQRTHTGEKPYKCPECGKSFSQLAHLRAHQRTHTGKKTS

SEQ ID NO: 629 - protein, 222 aa, synthetic construct
  REGION 1..222: note = source = /note="Description of Artificial Sequence: Syntheticpolypeptide"
LEPGEKPYKCPECGKSFSQRAHLERHQRTHTGEKPYKCPECGKSFSRSDELVRHQRTHTPNPHRRTDPSHKPFQYKCPECGKSFSDPGHLVRHQRTHTGEKPYKCPECGKSFSTKNSLTEHQRTHTGEKPYKCPECGKSFSRADNLTEHQRTHTHPRAPIPKPFQYKCPECGKSFSQRAHLERHQRTHTGEKPYKCPECGKSFSQRANLRAHQRTHTGKKTS

SEQ ID NO: 630 - protein, 222 aa, synthetic construct
  REGION 1..222: note = source = /note="Description of Artificial Sequence: Syntheticpolypeptide"
LEPGEKPYKCPECGKSFSDSGNLRVHQRTHTGEKPYKCPECGKSFSQRAHLERHQRTHTGEKPYKCPECGKSFSRSDELVRHQRTHTPNPHRRTDPSHKPFQYKCPECGKSFSDPGHLVRHQRTHTGEKPYKCPECGKSFSTKNSLTEHQRTHTGEKPYKCPECGKSFSRADNLTEHQRTHTHPRAPIPKPFQYKCPECGKSFSQRAHLERHQRTHTGKKTS

SEQ ID NO: 631 - protein, 218 aa, synthetic construct
  REGION 1..218: note = source = /note="Description of Artificial Sequence: Syntheticpolypeptide"
LEPGEKPYKCPECGKSFSDPGALVRHQRTHTHPRAPIPKPFQYKCPECGKSFSDSGNLRVHQRTHTGEKPYKCPECGKSFSQRAHLERHQRTHTGEKPYKCPECGKSFSRSDELVRHQRTHTHPRAPIPKPFQYKCPECGKSFSTSGELVRHQRTHTGEKPYKCPECGKSFSRNDALTEHQRTHTGEKPYKCPECGKSFSERSHLREHQRTHTGKKTS

SEQ ID NO: 632 - protein, 218 aa, synthetic construct
  REGION 1..218: note = source = /note="Description of Artificial Sequence: Syntheticpolypeptide"
LEPGEKPYKCPECGKSFSRTDTLRDHQRTHTGEKPYKCPECGKSFSDPGALVRHQRTHTHPRAPIPKPFQYKCPECGKSFSDSGNLRVHQRTHTGEKPYKCPECGKSFSQRAHLERHQRTHTGEKPYKCPECGKSFSRSDELVRHQRTHTHPRAPIPKPFQYKCPECGKSFSTSGELVRHQRTHTGEKPYKCPECGKSFSRNDALTEHQRTHTGKKTS

SEQ ID NO: 633 - protein, 211 aa, synthetic construct
  REGION 1..211: note = source = /note="Description of Artificial Sequence: Syntheticpolypeptide"
LEPGEKPYKCPECGKSFSDPGALVRHQRTHTHPRAPIPKPFQYKCPECGKSFSDSGNLRVHQRTHTGEKPYKCPECGKSFSQRAHLERHQRTHTGEKPYKCPECGKSFSRSDELVRHQRTHTGEKPYKCPECGKSFSTTGALTEHQRTHTGEKPYKCPECGKSFSRSDHLTTHQRTHTGEKPYKCPECGKSFSDCRDLARHQRTHTGKKTS

SEQ ID NO: 634 - protein, 211 aa, synthetic construct
  REGION 1..211: note = source = /note="Description of Artificial Sequence: Syntheticpolypeptide"
LEPGEKPYKCPECGKSFSRTDTLRDHQRTHTGEKPYKCPECGKSFSDPGALVRHQRTHTHPRAPIPKPFQYKCPECGKSFSDSGNLRVHQRTHTGEKPYKCPECGKSFSQRAHLERHQRTHTGEKPYKCPECGKSFSRSDELVRHQRTHTGEKPYKCPECGKSFSTTGALTEHQRTHTGEKPYKCPECGKSFSRSDHLTTHQRTHTGKKTS

SEQ ID NO: 635 - protein, 218 aa, synthetic construct
  REGION 1..218: note = source = /note="Description of Artificial Sequence: Syntheticpolypeptide"
LEPGEKPYKCPECGKSFSQRANLRAHQRTHTGEKPYKCPECGKSFSQSGNLTEHQRTHTHPRAPIPKPFQYKCPECGKSFSQSSSLVRHQRTHTGEKPYKCPECGKSFSSKKALTEHQRTHTHPRAPIPKPFQYKCPECGKSFSRNDALTEHQRTHTGEKPYKCPECGKSFSQRANLRAHQRTHTGEKPYKCPECGKSFSRSDKLVRHQRTHTGKKTS

SEQ ID NO: 636 - protein, 218 aa, synthetic construct
  REGION 1..218: note = source = /note="Description of Artificial Sequence: Syntheticpolypeptide"
LEPGEKPYKCPECGKSFSQSGNLTEHQRTHTGEKPYKCPECGKSFSQRANLRAHQRTHTGEKPYKCPECGKSFSQSGNLTEHQRTHTHPRAPIPKPFQYKCPECGKSFSQSSSLVRHQRTHTGEKPYKCPECGKSFSSKKALTEHQRTHTHPRAPIPKPFQYKCPECGKSFSRNDALTEHQRTHTGEKPYKCPECGKSFSQRANLRAHQRTHTGKKTS

SEQ ID NO: 637 - protein, 218 aa, synthetic construct
  REGION 1..218: note = source = /note="Description of Artificial Sequence: Syntheticpolypeptide"
LEPGEKPYKCPECGKSFSTTGALTEHQRTHTGEKPYKCPECGKSFSQSGNLTEHQRTHTGEKPYKCPECGKSFSQRANLRAHQRTHTGEKPYKCPECGKSFSQSGNLTEHQRTHTHPRAPIPKPFQYKCPECGKSFSQSSSLVRHQRTHTGEKPYKCPECGKSFSSKKALTEHQRTHTHPRAPIPKPFQYKCPECGKSFSRNDALTEHQRTHTGKKTS

SEQ ID NO: 638 - protein, 218 aa, synthetic construct
  REGION 1..218: note = source = /note="Description of Artificial Sequence: Syntheticpolypeptide"
LEPGEKPYKCPECGKSFSTSGELVRHQRTHTHPRAPIPKPFQYKCPECGKSFSTTGALTEHQRTHTGEKPYKCPECGKSFSQSGNLTEHQRTHTGEKPYKCPECGKSFSQRANLRAHQRTHTGEKPYKCPECGKSFSQSGNLTEHQRTHTHPRAPIPKPFQYKCPECGKSFSQSSSLVRHQRTHTGEKPYKCPECGKSFSSKKALTEHQRTHTGKKTS

SEQ ID NO: 639 - protein, 215 aa, synthetic construct
  REGION 1..215: note = source = /note="Description of Artificial Sequence: Syntheticpolypeptide"
LEPGEKPYKCPECGKSFSDKKDLTRHQRTHTGEKPYKCPECGKSFSQRANLRAHQRTHTGEKPYKCPECGKSFSQLAHLRAHQRTHTGEKPYKCPECGKSFSQLAHLRAHQRTHTGEKPYKCPECGKSFSRNDTLTEHQRTHTGEKPYKCPECGKSFSQKSSLIAHQRTHTPNPHRRTDPSHKPFQYKCPECGKSFSTTGALTEHQRTHTGKKTS

SEQ ID NO: 640 - protein, 204 aa, synthetic construct
  REGION 1..204: note = source = /note="Description of Artificial Sequence: Syntheticpolypeptide"
LEPGEKPYKCPECGKSFSRTDTLRDHQRTHTGEKPYKCPECGKSFSDKKDLTRHQRTHTGEKPYKCPECGKSFSQRANLRAHQRTHTGEKPYKCPECGKSFSQLAHLRAHQRTHTGEKPYKCPECGKSFSQLAHLRAHQRTHTGEKPYKCPECGKSFSRNDTLTEHQRTHTGEKPYKCPECGKSFSQKSSLIAHQRTHTGKKTS

SEQ ID NO: 641 - protein, 204 aa, synthetic construct
  REGION 1..204: note = source = /note="Description of Artificial Sequence: Syntheticpolypeptide"
LEPGEKPYKCPECGKSFSRNDALTEHQRTHTGEKPYKCPECGKSFSRTDTLRDHQRTHTGEKPYKCPECGKSFSDKKDLTRHQRTHTGEKPYKCPECGKSFSQRANLRAHQRTHTGEKPYKCPECGKSFSQLAHLRAHQRTHTGEKPYKCPECGKSFSQLAHLRAHQRTHTGEKPYKCPECGKSFSRNDTLTEHQRTHTGKKTS

SEQ ID NO: 642 - protein, 211 aa, synthetic construct
  REGION 1..211: note = source = /note="Description of Artificial Sequence: Syntheticpolypeptide"
LEPGEKPYKCPECGKSFSRTDTLRDHQRTHTHPRAPIPKPFQYKCPECGKSFSRNDALTEHQRTHTGEKPYKCPECGKSFSRTDTLRDHQRTHTGEKPYKCPECGKSFSDKKDLTRHQRTHTGEKPYKCPECGKSFSQRANLRAHQRTHTGEKPYKCPECGKSFSQLAHLRAHQRTHTGEKPYKCPECGKSFSQLAHLRAHQRTHTGKKTS

SEQ ID NO: 643 - protein, 211 aa, synthetic construct
  REGION 1..211: note = source = /note="Description of Artificial Sequence: Syntheticpolypeptide"
LEPGEKPYKCPECGKSFSRSDELVRHQRTHTGEKPYKCPECGKSFSRTDTLRDHQRTHTHPRAPIPKPFQYKCPECGKSFSRNDALTEHQRTHTGEKPYKCPECGKSFSRTDTLRDHQRTHTGEKPYKCPECGKSFSDKKDLTRHQRTHTGEKPYKCPECGKSFSQRANLRAHQRTHTGEKPYKCPECGKSFSQLAHLRAHQRTHTGKKTS

SEQ ID NO: 644 - protein, 211 aa, synthetic construct
  REGION 1..211: note = source = /note="Description of Artificial Sequence: Syntheticpolypeptide"
LEPGEKPYKCPECGKSFSRSDHLTTHQRTHTGEKPYKCPECGKSFSRSDELVRHQRTHTGEKPYKCPECGKSFSRTDTLRDHQRTHTHPRAPIPKPFQYKCPECGKSFSRNDALTEHQRTHTGEKPYKCPECGKSFSRTDTLRDHQRTHTGEKPYKCPECGKSFSDKKDLTRHQRTHTGEKPYKCPECGKSFSQRANLRAHQRTHTGKKTS

SEQ ID NO: 645 - protein, 211 aa, synthetic construct
  REGION 1..211: note = source = /note="Description of Artificial Sequence: Syntheticpolypeptide"
LEPGEKPYKCPECGKSFSRKDNLKNHQRTHTGEKPYKCPECGKSFSRSDHLTTHQRTHTGEKPYKCPECGKSFSRSDELVRHQRTHTGEKPYKCPECGKSFSRTDTLRDHQRTHTHPRAPIPKPFQYKCPECGKSFSRNDALTEHQRTHTGEKPYKCPECGKSFSRTDTLRDHQRTHTGEKPYKCPECGKSFSDKKDLTRHQRTHTGKKTS

SEQ ID NO: 646 - protein, 211 aa, synthetic construct
  REGION 1..211: note = source = /note="Description of Artificial Sequence: Syntheticpolypeptide"
LEPGEKPYKCPECGKSFSRNDTLTEHQRTHTGEKPYKCPECGKSFSRKDNLKNHQRTHTGEKPYKCPECGKSFSRSDHLTTHQRTHTGEKPYKCPECGKSFSRSDELVRHQRTHTGEKPYKCPECGKSFSRTDTLRDHQRTHTHPRAPIPKPFQYKCPECGKSFSRNDALTEHQRTHTGEKPYKCPECGKSFSRTDTLRDHQRTHTGKKTS

SEQ ID NO: 647 - protein, 211 aa, synthetic construct
  REGION 1..211: note = source = /note="Description of Artificial Sequence: Syntheticpolypeptide"
LEPGEKPYKCPECGKSFSRADNLTEHQRTHTGEKPYKCPECGKSFSRNDTLTEHQRTHTGEKPYKCPECGKSFSRKDNLKNHQRTHTGEKPYKCPECGKSFSRSDHLTTHQRTHTGEKPYKCPECGKSFSRSDELVRHQRTHTGEKPYKCPECGKSFSRTDTLRDHQRTHTHPRAPIPKPFQYKCPECGKSFSRNDALTEHQRTHTGKKTS

SEQ ID NO: 648 - protein, 204 aa, synthetic construct
  REGION 1..204: note = source = /note="Description of Artificial Sequence: Syntheticpolypeptide"
LEPGEKPYKCPECGKSFSTSGELVRHQRTHTGEKPYKCPECGKSFSSKKALTEHQRTHTGEKPYKCPECGKSFSDSGNLRVHQRTHTGEKPYKCPECGKSFSQRANLRAHQRTHTGEKPYKCPECGKSFSRKDNLKNHQRTHTGEKPYKCPECGKSFSRSDNLVRHQRTHTGEKPYKCPECGKSFSDKKDLTRHQRTHTGKKTS

SEQ ID NO: 649 - protein, 204 aa, synthetic construct
  REGION 1..204: note = source = /note="Description of Artificial Sequence: Syntheticpolypeptide"
LEPGEKPYKCPECGKSFSQSSSLVRHQRTHTGEKPYKCPECGKSFSTSGELVRHQRTHTGEKPYKCPECGKSFSSKKALTEHQRTHTGEKPYKCPECGKSFSDSGNLRVHQRTHTGEKPYKCPECGKSFSQRANLRAHQRTHTGEKPYKCPECGKSFSRKDNLKNHQRTHTGEKPYKCPECGKSFSRSDNLVRHQRTHTGKKTS

SEQ ID NO: 650 - protein, 204 aa, synthetic construct
  REGION 1..204: note = source = /note="Description of Artificial Sequence: Syntheticpolypeptide"
LEPGEKPYKCPECGKSFSRSDKLTEHQRTHTGEKPYKCPECGKSFSQSSSLVRHQRTHTGEKPYKCPECGKSFSTSGELVRHQRTHTGEKPYKCPECGKSFSSKKALTEHQRTHTGEKPYKCPECGKSFSDSGNLRVHQRTHTGEKPYKCPECGKSFSQRANLRAHQRTHTGEKPYKCPECGKSFSRKDNLKNHQRTHTGKKTS

SEQ ID NO: 651 - protein, 211 aa, synthetic construct
  REGION 1..211: note = source = /note="Description of Artificial Sequence: Syntheticpolypeptide"
LEPGEKPYKCPECGKSFSRSDELVRHQRTHTHPRAPIPKPFQYKCPECGKSFSRSDKLTEHQRTHTGEKPYKCPECGKSFSQSSSLVRHQRTHTGEKPYKCPECGKSFSTSGELVRHQRTHTGEKPYKCPECGKSFSSKKALTEHQRTHTGEKPYKCPECGKSFSDSGNLRVHQRTHTGEKPYKCPECGKSFSQRANLRAHQRTHTGKKTS

SEQ ID NO: 652 - protein, 211 aa, synthetic construct
  REGION 1..211: note = source = /note="Description of Artificial Sequence: Syntheticpolypeptide"
LEPGEKPYKCPECGKSFSQSSNLVRHQRTHTGEKPYKCPECGKSFSRSDELVRHQRTHTHPRAPIPKPFQYKCPECGKSFSRSDKLTEHQRTHTGEKPYKCPECGKSFSQSSSLVRHQRTHTGEKPYKCPECGKSFSTSGELVRHQRTHTGEKPYKCPECGKSFSSKKALTEHQRTHTGEKPYKCPECGKSFSDSGNLRVHQRTHTGKKTS

SEQ ID NO: 653 - protein, 211 aa, synthetic construct
  REGION 1..211: note = source = /note="Description of Artificial Sequence: Syntheticpolypeptide"
LEPGEKPYKCPECGKSFSDCRDLARHQRTHTGEKPYKCPECGKSFSQSSNLVRHQRTHTGEKPYKCPECGKSFSRSDELVRHQRTHTHPRAPIPKPFQYKCPECGKSFSRSDKLTEHQRTHTGEKPYKCPECGKSFSQSSSLVRHQRTHTGEKPYKCPECGKSFSTSGELVRHQRTHTGEKPYKCPECGKSFSSKKALTEHQRTHTGKKTS

SEQ ID NO: 654 - protein, 211 aa, synthetic construct
  REGION 1..211: note = source = /note="Description of Artificial Sequence: Syntheticpolypeptide"
LEPGEKPYKCPECGKSFSQSGDLRRHQRTHTGEKPYKCPECGKSFSDCRDLARHQRTHTGEKPYKCPECGKSFSQSSNLVRHQRTHTGEKPYKCPECGKSFSRSDELVRHQRTHTHPRAPIPKPFQYKCPECGKSFSRSDKLTEHQRTHTGEKPYKCPECGKSFSQSSSLVRHQRTHTGEKPYKCPECGKSFSTSGELVRHQRTHTGKKTS

SEQ ID NO: 655 - protein, 211 aa, synthetic construct
  REGION 1..211: note = source = /note="Description of Artificial Sequence: Syntheticpolypeptide"
LEPGEKPYKCPECGKSFSDPGNLVRHQRTHTGEKPYKCPECGKSFSQSGDLRRHQRTHTGEKPYKCPECGKSFSDCRDLARHQRTHTGEKPYKCPECGKSFSQSSNLVRHQRTHTGEKPYKCPECGKSFSRSDELVRHQRTHTHPRAPIPKPFQYKCPECGKSFSRSDKLTEHQRTHTGEKPYKCPECGKSFSQSSSLVRHQRTHTGKKTS

SEQ ID NO: 656 - protein, 211 aa, synthetic construct
  REGION 1..211: note = source = /note="Description of Artificial Sequence: Syntheticpolypeptide"
LEPGEKPYKCPECGKSFSQSSSLVRHQRTHTHPRAPIPKPFQYKCPECGKSFSHTGHLLEHQRTHTGEKPYKCPECGKSFSTSHSLTEHQRTHTGEKPYKCPECGKSFSQRANLRAHQRTHTGEKPYKCPECGKSFSQSSNLVRHQRTHTGEKPYKCPECGKSFSQSSNLVRHQRTHTGEKPYKCPECGKSFSQSGHLTEHQRTHTGKKTS

SEQ ID NO: 657 - protein, 211 aa, synthetic construct
  REGION 1..211: note = source = /note="Description of Artificial Sequence: Syntheticpolypeptide"
LEPGEKPYKCPECGKSFSRSDKLTEHQRTHTGEKPYKCPECGKSFSQSSSLVRHQRTHTHPRAPIPKPFQYKCPECGKSFSHTGHLLEHQRTHTGEKPYKCPECGKSFSTSHSLTEHQRTHTGEKPYKCPECGKSFSQRANLRAHQRTHTGEKPYKCPECGKSFSQSSNLVRHQRTHTGEKPYKCPECGKSFSQSSNLVRHQRTHTGKKTS

SEQ ID NO: 658 - protein, 218 aa, synthetic construct
  REGION 1..218: note = source = /note="Description of Artificial Sequence: Syntheticpolypeptide"
LEPGEKPYKCPECGKSFSRSDELVRHQRTHTHPRAPIPKPFQYKCPECGKSFSRSDKLTEHQRTHTGEKPYKCPECGKSFSQSSSLVRHQRTHTHPRAPIPKPFQYKCPECGKSFSHTGHLLEHQRTHTGEKPYKCPECGKSFSTSHSLTEHQRTHTGEKPYKCPECGKSFSQRANLRAHQRTHTGEKPYKCPECGKSFSQSSNLVRHQRTHTGKKTS

SEQ ID NO: 659 - protein, 218 aa, synthetic construct
  REGION 1..218: note = source = /note="Description of Artificial Sequence: Syntheticpolypeptide"
LEPGEKPYKCPECGKSFSQSSNLVRHQRTHTGEKPYKCPECGKSFSRSDELVRHQRTHTHPRAPIPKPFQYKCPECGKSFSRSDKLTEHQRTHTGEKPYKCPECGKSFSQSSSLVRHQRTHTHPRAPIPKPFQYKCPECGKSFSHTGHLLEHQRTHTGEKPYKCPECGKSFSTSHSLTEHQRTHTGEKPYKCPECGKSFSQRANLRAHQRTHTGKKTS

SEQ ID NO: 660 - protein, 218 aa, synthetic construct
  REGION 1..218: note = source = /note="Description of Artificial Sequence: Syntheticpolypeptide"
LEPGEKPYKCPECGKSFSDCRDLARHQRTHTGEKPYKCPECGKSFSQSSNLVRHQRTHTGEKPYKCPECGKSFSRSDELVRHQRTHTHPRAPIPKPFQYKCPECGKSFSRSDKLTEHQRTHTGEKPYKCPECGKSFSQSSSLVRHQRTHTHPRAPIPKPFQYKCPECGKSFSHTGHLLEHQRTHTGEKPYKCPECGKSFSTSHSLTEHQRTHTGKKTS

SEQ ID NO: 661 - protein, 218 aa, synthetic construct
  REGION 1..218: note = source = /note="Description of Artificial Sequence: Syntheticpolypeptide"
LEPGEKPYKCPECGKSFSQSGDLRRHQRTHTGEKPYKCPECGKSFSDCRDLARHQRTHTGEKPYKCPECGKSFSQSSNLVRHQRTHTGEKPYKCPECGKSFSRSDELVRHQRTHTHPRAPIPKPFQYKCPECGKSFSRSDKLTEHQRTHTGEKPYKCPECGKSFSQSSSLVRHQRTHTHPRAPIPKPFQYKCPECGKSFSHTGHLLEHQRTHTGKKTS

SEQ ID NO: 662 - protein, 204 aa, synthetic construct
  REGION 1..204: note = source = /note="Description of Artificial Sequence: Syntheticpolypeptide"
LEPGEKPYKCPECGKSFSDKKDLTRHQRTHTGEKPYKCPECGKSFSRADNLTEHQRTHTGEKPYKCPECGKSFSRNDTLTEHQRTHTGEKPYKCPECGKSFSRKDNLKNHQRTHTGEKPYKCPECGKSFSRSDHLTTHQRTHTGEKPYKCPECGKSFSRSDELVRHQRTHTGEKPYKCPECGKSFSRTDTLRDHQRTHTGKKTS

SEQ ID NO: 663 - protein, 218 aa, synthetic construct
  REGION 1..218: note = source = /note="Description of Artificial Sequence: Syntheticpolypeptide"
LEPGEKPYKCPECGKSFSTTGNLTVHQRTHTHPRAPIPKPFQYKCPECGKSFSQAGHLASHQRTHTGEKPYKCPECGKSFSHTGHLLEHQRTHTGEKPYKCPECGKSFSRSDELVRHQRTHTHPRAPIPKPFQYKCPECGKSFSRSDELVRHQRTHTGEKPYKCPECGKSFSQAGHLASHQRTHTGEKPYKCPECGKSFSQSGHLTEHQRTHTGKKTS

SEQ ID NO: 664 - protein, 218 aa, synthetic construct
  REGION 1..218: note = source = /note="Description of Artificial Sequence: Syntheticpolypeptide"
LEPGEKPYKCPECGKSFSSKKALTEHQRTHTGEKPYKCPECGKSFSTTGNLTVHQRTHTHPRAPIPKPFQYKCPECGKSFSQAGHLASHQRTHTGEKPYKCPECGKSFSHTGHLLEHQRTHTGEKPYKCPECGKSFSRSDELVRHQRTHTHPRAPIPKPFQYKCPECGKSFSRSDELVRHQRTHTGEKPYKCPECGKSFSQAGHLASHQRTHTGKKTS

SEQ ID NO: 665 - protein, 218 aa, synthetic construct
  REGION 1..218: note = source = /note="Description of Artificial Sequence: Syntheticpolypeptide"
LEPGEKPYKCPECGKSFSRSDKLVRHQRTHTGEKPYKCPECGKSFSSKKALTEHQRTHTGEKPYKCPECGKSFSTTGNLTVHQRTHTHPRAPIPKPFQYKCPECGKSFSQAGHLASHQRTHTGEKPYKCPECGKSFSHTGHLLEHQRTHTGEKPYKCPECGKSFSRSDELVRHQRTHTHPRAPIPKPFQYKCPECGKSFSRSDELVRHQRTHTGKKTS

SEQ ID NO: 666 - protein, 229 aa, synthetic construct
  REGION 1..229: note = source = /note="Description of Artificial Sequence: Syntheticpolypeptide"
LEPGEKPYKCPECGKSFSSKKALTEHQRTHTPNPHRRTDPSHKPFQYKCPECGKSFSQKSSLIAHQRTHTGEKPYKCPECGKSFSRNDALTEHQRTHTGEKPYKCPECGKSFSRSDDLVRHQRTHTHPRAPIPKPFQYKCPECGKSFSRSDELVRHQRTHTHPRAPIPKPFQYKCPECGKSFSRSDNLVRHQRTHTGEKPYKCPECGKSFSTSGNLVRHQRTHTGKKTS

SEQ ID NO: 667 - protein, 229 aa, synthetic construct
  REGION 1..229: note = source = /note="Description of Artificial Sequence: Syntheticpolypeptide"
LEPGEKPYKCPECGKSFSRSDKLVRHQRTHTGEKPYKCPECGKSFSSKKALTEHQRTHTPNPHRRTDPSHKPFQYKCPECGKSFSQKSSLIAHQRTHTGEKPYKCPECGKSFSRNDALTEHQRTHTGEKPYKCPECGKSFSRSDDLVRHQRTHTHPRAPIPKPFQYKCPECGKSFSRSDELVRHQRTHTHPRAPIPKPFQYKCPECGKSFSRSDNLVRHQRTHTGKKTS

SEQ ID NO: 668 - protein, 222 aa, synthetic construct
  REGION 1..222: note = source = /note="Description of Artificial Sequence: Syntheticpolypeptide"
LEPGEKPYKCPECGKSFSSKKALTEHQRTHTPNPHRRTDPSHKPFQYKCPECGKSFSQKSSLIAHQRTHTGEKPYKCPECGKSFSRNDALTEHQRTHTGEKPYKCPECGKSFSRSDDLVRHQRTHTHPRAPIPKPFQYKCPECGKSFSRSDELVRHQRTHTGEKPYKCPECGKSFSHRTTLTNHQRTHTGEKPYKCPECGKSFSRRDELNVHQRTHTGKKTS

SEQ ID NO: 669 - protein, 222 aa, synthetic construct
  REGION 1..222: note = source = /note="Description of Artificial Sequence: Syntheticpolypeptide"
LEPGEKPYKCPECGKSFSRSDKLVRHQRTHTGEKPYKCPECGKSFSSKKALTEHQRTHTPNPHRRTDPSHKPFQYKCPECGKSFSQKSSLIAHQRTHTGEKPYKCPECGKSFSRNDALTEHQRTHTGEKPYKCPECGKSFSRSDDLVRHQRTHTHPRAPIPKPFQYKCPECGKSFSRSDELVRHQRTHTGEKPYKCPECGKSFSHRTTLTNHQRTHTGKKTS

SEQ ID NO: 670 - protein, 222 aa, synthetic construct
  REGION 1..222: note = source = /note="Description of Artificial Sequence: Syntheticpolypeptide"
LEPGEKPYKCPECGKSFSRSDELVRHQRTHTGEKPYKCPECGKSFSRSDKLVRHQRTHTGEKPYKCPECGKSFSSKKALTEHQRTHTPNPHRRTDPSHKPFQYKCPECGKSFSQKSSLIAHQRTHTGEKPYKCPECGKSFSRNDALTEHQRTHTGEKPYKCPECGKSFSRSDDLVRHQRTHTHPRAPIPKPFQYKCPECGKSFSRSDELVRHQRTHTGKKTS

SEQ ID NO: 671 - protein, 211 aa, synthetic construct
  REGION 1..211: note = source = /note="Description of Artificial Sequence: Syntheticpolypeptide"
LEPGEKPYKCPECGKSFSRSDELVRHQRTHTGEKPYKCPECGKSFSRSDKLVRHQRTHTGEKPYKCPECGKSFSSKKALTEHQRTHTGEKPYKCPECGKSFSTTGNLTVHQRTHTHPRAPIPKPFQYKCPECGKSFSQAGHLASHQRTHTGEKPYKCPECGKSFSHTGHLLEHQRTHTGEKPYKCPECGKSFSRSDELVRHQRTHTGKKTS

SEQ ID NO: 672 - protein, 211 aa, synthetic construct
  REGION 1..211: note = source = /note="Description of Artificial Sequence: Syntheticpolypeptide"
LEPGEKPYKCPECGKSFSRSDKLVRHQRTHTGEKPYKCPECGKSFSRSDELVRHQRTHTGEKPYKCPECGKSFSRSDKLVRHQRTHTGEKPYKCPECGKSFSSKKALTEHQRTHTGEKPYKCPECGKSFSTTGNLTVHQRTHTHPRAPIPKPFQYKCPECGKSFSQAGHLASHQRTHTGEKPYKCPECGKSFSHTGHLLEHQRTHTGKKTS

SEQ ID NO: 673 - protein, 211 aa, synthetic construct
  REGION 1..211: note = source = /note="Description of Artificial Sequence: Syntheticpolypeptide"
LEPGEKPYKCPECGKSFSRSDKLVRHQRTHTGEKPYKCPECGKSFSRSDKLVRHQRTHTGEKPYKCPECGKSFSRSDELVRHQRTHTGEKPYKCPECGKSFSRSDKLVRHQRTHTGEKPYKCPECGKSFSSKKALTEHQRTHTGEKPYKCPECGKSFSTTGNLTVHQRTHTHPRAPIPKPFQYKCPECGKSFSQAGHLASHQRTHTGKKTS

SEQ ID NO: 674 - protein, 215 aa, synthetic construct
  REGION 1..215: note = source = /note="Description of Artificial Sequence: Syntheticpolypeptide"
LEPGEKPYKCPECGKSFSRSDKLVRHQRTHTGEKPYKCPECGKSFSRSDELVRHQRTHTGEKPYKCPECGKSFSRSDKLVRHQRTHTGEKPYKCPECGKSFSSKKALTEHQRTHTPNPHRRTDPSHKPFQYKCPECGKSFSQKSSLIAHQRTHTGEKPYKCPECGKSFSRNDALTEHQRTHTGEKPYKCPECGKSFSRSDDLVRHQRTHTGKKTS

SEQ ID NO: 675 - protein, 215 aa, synthetic construct
  REGION 1..215: note = source = /note="Description of Artificial Sequence: Syntheticpolypeptide"
LEPGEKPYKCPECGKSFSRSDKLVRHQRTHTGEKPYKCPECGKSFSRSDKLVRHQRTHTGEKPYKCPECGKSFSRSDELVRHQRTHTGEKPYKCPECGKSFSRSDKLVRHQRTHTGEKPYKCPECGKSFSSKKALTEHQRTHTPNPHRRTDPSHKPFQYKCPECGKSFSQKSSLIAHQRTHTGEKPYKCPECGKSFSRNDALTEHQRTHTGKKTS

SEQ ID NO: 676 - protein, 215 aa, synthetic construct
  REGION 1..215: note = source = /note="Description of Artificial Sequence: Syntheticpolypeptide"
LEPGEKPYKCPECGKSFSTSGHLVRHQRTHTGEKPYKCPECGKSFSRSDKLVRHQRTHTGEKPYKCPECGKSFSRSDKLVRHQRTHTGEKPYKCPECGKSFSRSDELVRHQRTHTGEKPYKCPECGKSFSRSDKLVRHQRTHTGEKPYKCPECGKSFSSKKALTEHQRTHTPNPHRRTDPSHKPFQYKCPECGKSFSQKSSLIAHQRTHTGKKTS

SEQ ID NO: 677 - protein, 204 aa, synthetic construct
  REGION 1..204: note = source = /note="Description of Artificial Sequence: Syntheticpolypeptide"
LEPGEKPYKCPECGKSFSRSDKLVRHQRTHTGEKPYKCPECGKSFSRSDELVRHQRTHTGEKPYKCPECGKSFSRSDKLVRHQRTHTGEKPYKCPECGKSFSSKKALTEHQRTHTGEKPYKCPECGKSFSTTGNLTVHQRTHTGEKPYKCPECGKSFSTSGNLVRHQRTHTGEKPYKCPECGKSFSTSGELVRHQRTHTGKKTS

SEQ ID NO: 678 - protein, 204 aa, synthetic construct
  REGION 1..204: note = source = /note="Description of Artificial Sequence: Syntheticpolypeptide"
LEPGEKPYKCPECGKSFSRSDKLVRHQRTHTGEKPYKCPECGKSFSRSDKLVRHQRTHTGEKPYKCPECGKSFSRSDELVRHQRTHTGEKPYKCPECGKSFSRSDKLVRHQRTHTGEKPYKCPECGKSFSSKKALTEHQRTHTGEKPYKCPECGKSFSTTGNLTVHQRTHTGEKPYKCPECGKSFSTSGNLVRHQRTHTGKKTS

SEQ ID NO: 679 - protein, 204 aa, synthetic construct
  REGION 1..204: note = source = /note="Description of Artificial Sequence: Syntheticpolypeptide"
LEPGEKPYKCPECGKSFSTSGHLVRHQRTHTGEKPYKCPECGKSFSRSDKLVRHQRTHTGEKPYKCPECGKSFSRSDKLVRHQRTHTGEKPYKCPECGKSFSRSDELVRHQRTHTGEKPYKCPECGKSFSRSDKLVRHQRTHTGEKPYKCPECGKSFSSKKALTEHQRTHTGEKPYKCPECGKSFSTTGNLTVHQRTHTGKKTS

SEQ ID NO: 680 - protein, 215 aa, synthetic construct
  REGION 1..215: note = source = /note="Description of Artificial Sequence: Syntheticpolypeptide"
LEPGEKPYKCPECGKSFSTSGNLTEHQRTHTPNPHRRTDPSHKPFQYKCPECGKSFSTSGSLVRHQRTHTGEKPYKCPECGKSFSRSDKLVRHQRTHTGEKPYKCPECGKSFSRSDKLVRHQRTHTGEKPYKCPECGKSFSRSDHLTTHQRTHTGEKPYKCPECGKSFSRSDKLVRHQRTHTGEKPYKCPECGKSFSRTDTLRDHQRTHTGKKTS

SEQ ID NO: 681 - protein, 215 aa, synthetic construct
  REGION 1..215: note = source = /note="Description of Artificial Sequence: Syntheticpolypeptide"
LEPGEKPYKCPECGKSFSHRTTLTNHQRTHTGEKPYKCPECGKSFSTSGNLTEHQRTHTPNPHRRTDPSHKPFQYKCPECGKSFSTSGSLVRHQRTHTGEKPYKCPECGKSFSRSDKLVRHQRTHTGEKPYKCPECGKSFSRSDKLVRHQRTHTGEKPYKCPECGKSFSRSDHLTTHQRTHTGEKPYKCPECGKSFSRSDKLVRHQRTHTGKKTS

SEQ ID NO: 682 - protein, 222 aa, synthetic construct
  REGION 1..222: note = source = /note="Description of Artificial Sequence: Syntheticpolypeptide"
LEPGEKPYKCPECGKSFSQRANLRAHQRTHTGEKPYKCPECGKSFSTTGALTEHQRTHTGEKPYKCPECGKSFSQLAHLRAHQRTHTGEKPYKCPECGKSFSRRDELNVHQRTHTHPRAPIPKPFQYKCPECGKSFSTSGNLVRHQRTHTGEKPYKCPECGKSFSTTGALTEHQRTHTPNPHRRTDPSHKPFQYKCPECGKSFSTTGALTEHQRTHTGKKTS

SEQ ID NO: 683 - protein, 211 aa, synthetic construct
  REGION 1..211: note = source = /note="Description of Artificial Sequence: Syntheticpolypeptide"
LEPGEKPYKCPECGKSFSRSDKLTEHQRTHTGEKPYKCPECGKSFSQRANLRAHQRTHTGEKPYKCPECGKSFSTTGALTEHQRTHTGEKPYKCPECGKSFSQLAHLRAHQRTHTGEKPYKCPECGKSFSRRDELNVHQRTHTHPRAPIPKPFQYKCPECGKSFSTSGNLVRHQRTHTGEKPYKCPECGKSFSTTGALTEHQRTHTGKKTS

SEQ ID NO: 684 - protein, 211 aa, synthetic construct
  REGION 1..211: note = source = /note="Description of Artificial Sequence: Syntheticpolypeptide"
LEPGEKPYKCPECGKSFSQRANLRAHQRTHTGEKPYKCPECGKSFSRSDKLTEHQRTHTGEKPYKCPECGKSFSQRANLRAHQRTHTGEKPYKCPECGKSFSTTGALTEHQRTHTGEKPYKCPECGKSFSQLAHLRAHQRTHTGEKPYKCPECGKSFSRRDELNVHQRTHTHPRAPIPKPFQYKCPECGKSFSTSGNLVRHQRTHTGKKTS

SEQ ID NO: 685 - protein, 215 aa, synthetic construct
  REGION 1..215: note = source = /note="Description of Artificial Sequence: Syntheticpolypeptide"
LEPGEKPYKCPECGKSFSQRANLRAHQRTHTGEKPYKCPECGKSFSRSDKLTEHQRTHTGEKPYKCPECGKSFSQRANLRAHQRTHTGEKPYKCPECGKSFSTTGALTEHQRTHTGEKPYKCPECGKSFSQLAHLRAHQRTHTGEKPYKCPECGKSFSRRDELNVHQRTHTPNPHRRTDPSHKPFQYKCPECGKSFSQAGHLASHQRTHTGKKTS

SEQ ID NO: 686 - protein, 204 aa, synthetic construct
  REGION 1..204: note = source = /note="Description of Artificial Sequence: Syntheticpolypeptide"
LEPGEKPYKCPECGKSFSQRANLRAHQRTHTGEKPYKCPECGKSFSRSDKLTEHQRTHTGEKPYKCPECGKSFSQRANLRAHQRTHTGEKPYKCPECGKSFSTTGALTEHQRTHTGEKPYKCPECGKSFSQLAHLRAHQRTHTGEKPYKCPECGKSFSRRDELNVHQRTHTGEKPYKCPECGKSFSHKNALQNHQRTHTGKKTS

SEQ ID NO: 687 - protein, 226 aa, synthetic construct
  REGION 1..226: note = source = /note="Description of Artificial Sequence: Syntheticpolypeptide"
LEPGEKPYKCPECGKSFSTSGSLVRHQRTHTGEKPYKCPECGKSFSRSDHLTTHQRTHTPNPHRRTDPSHKPFQYKCPECGKSFSTTGNLTVHQRTHTGEKPYKCPECGKSFSQAGHLASHQRTHTPNPHRRTDPSHKPFQYKCPECGKSFSTTGNLTVHQRTHTGEKPYKCPECGKSFSQRAHLERHQRTHTGEKPYKCPECGKSFSDSGNLRVHQRTHTGKKTS

SEQ ID NO: 688 - protein, 222 aa, synthetic construct
  REGION 1..222: note = source = /note="Description of Artificial Sequence: Syntheticpolypeptide"
LEPGEKPYKCPECGKSFSTSGSLVRHQRTHTGEKPYKCPECGKSFSRSDHLTTHQRTHTPNPHRRTDPSHKPFQYKCPECGKSFSTTGNLTVHQRTHTGEKPYKCPECGKSFSQAGHLASHQRTHTHPRAPIPKPFQYKCPECGKSFSHKNALQNHQRTHTGEKPYKCPECGKSFSQSSNLVRHQRTHTGEKPYKCPECGKSFSRTDTLRDHQRTHTGKKTS

SEQ ID NO: 689 - protein, 218 aa, synthetic construct
  REGION 1..218: note = source = /note="Description of Artificial Sequence: Syntheticpolypeptide"
LEPGEKPYKCPECGKSFSRNDALTEHQRTHTGEKPYKCPECGKSFSTSGNLTEHQRTHTGEKPYKCPECGKSFSHRTTLTNHQRTHTGEKPYKCPECGKSFSRTDTLRDHQRTHTHPRAPIPKPFQYKCPECGKSFSTTGALTEHQRTHTGEKPYKCPECGKSFSDCRDLARHQRTHTHPRAPIPKPFQYKCPECGKSFSTTGALTEHQRTHTGKKTS

SEQ ID NO: 690 - protein, 211 aa, synthetic construct
  REGION 1..211: note = source = /note="Description of Artificial Sequence: Syntheticpolypeptide"
LEPGEKPYKCPECGKSFSQRANLRAHQRTHTGEKPYKCPECGKSFSRNDALTEHQRTHTGEKPYKCPECGKSFSTSGNLTEHQRTHTGEKPYKCPECGKSFSHRTTLTNHQRTHTGEKPYKCPECGKSFSRTDTLRDHQRTHTHPRAPIPKPFQYKCPECGKSFSTTGALTEHQRTHTGEKPYKCPECGKSFSDCRDLARHQRTHTGKKTS

SEQ ID NO: 691 - protein, 211 aa, synthetic construct
  REGION 1..211: note = source = /note="Description of Artificial Sequence: Syntheticpolypeptide"
LEPGEKPYKCPECGKSFSQKSSLIAHQRTHTGEKPYKCPECGKSFSQRANLRAHQRTHTGEKPYKCPECGKSFSRNDALTEHQRTHTGEKPYKCPECGKSFSTSGNLTEHQRTHTGEKPYKCPECGKSFSHRTTLTNHQRTHTGEKPYKCPECGKSFSRTDTLRDHQRTHTHPRAPIPKPFQYKCPECGKSFSTTGALTEHQRTHTGKKTS

SEQ ID NO: 692 - protein, 215 aa, synthetic construct
  REGION 1..215: note = source = /note="Description of Artificial Sequence: Syntheticpolypeptide"
LEPGEKPYKCPECGKSFSQKSSLIAHQRTHTGEKPYKCPECGKSFSQRANLRAHQRTHTGEKPYKCPECGKSFSRNDALTEHQRTHTGEKPYKCPECGKSFSTSGNLTEHQRTHTGEKPYKCPECGKSFSHRTTLTNHQRTHTGEKPYKCPECGKSFSRTDTLRDHQRTHTPNPHRRTDPSHKPFQYKCPECGKSFSTKNSLTEHQRTHTGKKTS

SEQ ID NO: 693 - protein, 204 aa, synthetic construct
  REGION 1..204: note = source = /note="Description of Artificial Sequence: Syntheticpolypeptide"
LEPGEKPYKCPECGKSFSQAGHLASHQRTHTGEKPYKCPECGKSFSQKSSLIAHQRTHTGEKPYKCPECGKSFSQRANLRAHQRTHTGEKPYKCPECGKSFSRNDALTEHQRTHTGEKPYKCPECGKSFSTSGNLTEHQRTHTGEKPYKCPECGKSFSHRTTLTNHQRTHTGEKPYKCPECGKSFSRTDTLRDHQRTHTGKKTS

SEQ ID NO: 694 - protein, 229 aa, synthetic construct
  REGION 1..229: note = source = /note="Description of Artificial Sequence: Syntheticpolypeptide"
LEPGEKPYKCPECGKSFSSKKALTEHQRTHTPNPHRRTDPSHKPFQYKCPECGKSFSSKKHLAEHQRTHTGEKPYKCPECGKSFSTTGALTEHQRTHTHPRAPIPKPFQYKCPECGKSFSHKNALQNHQRTHTGEKPYKCPECGKSFSRRDELNVHQRTHTHPRAPIPKPFQYKCPECGKSFSQRANLRAHQRTHTGEKPYKCPECGKSFSQAGHLASHQRTHTGKKTS

SEQ ID NO: 695 - protein, 229 aa, synthetic construct
  REGION 1..229: note = source = /note="Description of Artificial Sequence: Syntheticpolypeptide"
LEPGEKPYKCPECGKSFSSKKALTEHQRTHTGEKPYKCPECGKSFSSKKALTEHQRTHTPNPHRRTDPSHKPFQYKCPECGKSFSSKKHLAEHQRTHTGEKPYKCPECGKSFSTTGALTEHQRTHTHPRAPIPKPFQYKCPECGKSFSHKNALQNHQRTHTGEKPYKCPECGKSFSRRDELNVHQRTHTHPRAPIPKPFQYKCPECGKSFSQRANLRAHQRTHTGKKTS

SEQ ID NO: 696 - protein, 222 aa, synthetic construct
  REGION 1..222: note = source = /note="Description of Artificial Sequence: Syntheticpolypeptide"
LEPGEKPYKCPECGKSFSSKKALTEHQRTHTPNPHRRTDPSHKPFQYKCPECGKSFSSKKHLAEHQRTHTGEKPYKCPECGKSFSTTGALTEHQRTHTHPRAPIPKPFQYKCPECGKSFSHKNALQNHQRTHTGEKPYKCPECGKSFSRRDELNVHQRTHTGEKPYKCPECGKSFSTTGNLTVHQRTHTGEKPYKCPECGKSFSQSSNLVRHQRTHTGKKTS

SEQ ID NO: 697 - protein, 222 aa, synthetic construct
  REGION 1..222: note = source = /note="Description of Artificial Sequence: Syntheticpolypeptide"
LEPGEKPYKCPECGKSFSSKKALTEHQRTHTGEKPYKCPECGKSFSSKKALTEHQRTHTPNPHRRTDPSHKPFQYKCPECGKSFSSKKHLAEHQRTHTGEKPYKCPECGKSFSTTGALTEHQRTHTHPRAPIPKPFQYKCPECGKSFSHKNALQNHQRTHTGEKPYKCPECGKSFSRRDELNVHQRTHTGEKPYKCPECGKSFSTTGNLTVHQRTHTGKKTS

SEQ ID NO: 698 - protein, 222 aa, synthetic construct
  REGION 1..222: note = source = /note="Description of Artificial Sequence: Syntheticpolypeptide"
LEPGEKPYKCPECGKSFSQLAHLRAHQRTHTGEKPYKCPECGKSFSSKKALTEHQRTHTGEKPYKCPECGKSFSSKKALTEHQRTHTPNPHRRTDPSHKPFQYKCPECGKSFSSKKHLAEHQRTHTGEKPYKCPECGKSFSTTGALTEHQRTHTHPRAPIPKPFQYKCPECGKSFSHKNALQNHQRTHTGEKPYKCPECGKSFSRRDELNVHQRTHTGKKTS

SEQ ID NO: 699 - protein, 222 aa, synthetic construct
  REGION 1..222: note = source = /note="Description of Artificial Sequence: Syntheticpolypeptide"
LEPGEKPYKCPECGKSFSTSGHLVRHQRTHTGEKPYKCPECGKSFSQLAHLRAHQRTHTGEKPYKCPECGKSFSSKKALTEHQRTHTGEKPYKCPECGKSFSSKKALTEHQRTHTPNPHRRTDPSHKPFQYKCPECGKSFSSKKHLAEHQRTHTGEKPYKCPECGKSFSTTGALTEHQRTHTHPRAPIPKPFQYKCPECGKSFSHKNALQNHQRTHTGKKTS

SEQ ID NO: 700 - protein, 226 aa, synthetic construct
  REGION 1..226: note = source = /note="Description of Artificial Sequence: Syntheticpolypeptide"
LEPGEKPYKCPECGKSFSTSGHLVRHQRTHTGEKPYKCPECGKSFSQLAHLRAHQRTHTGEKPYKCPECGKSFSSKKALTEHQRTHTGEKPYKCPECGKSFSSKKALTEHQRTHTPNPHRRTDPSHKPFQYKCPECGKSFSSKKHLAEHQRTHTGEKPYKCPECGKSFSTTGALTEHQRTHTPNPHRRTDPSHKPFQYKCPECGKSFSTSGNLVRHQRTHTGKKTS

SEQ ID NO: 701 - protein, 215 aa, synthetic construct
  REGION 1..215: note = source = /note="Description of Artificial Sequence: Syntheticpolypeptide"
LEPGEKPYKCPECGKSFSRSDNLVRHQRTHTGEKPYKCPECGKSFSTSGHLVRHQRTHTGEKPYKCPECGKSFSQLAHLRAHQRTHTGEKPYKCPECGKSFSSKKALTEHQRTHTGEKPYKCPECGKSFSSKKALTEHQRTHTPNPHRRTDPSHKPFQYKCPECGKSFSSKKHLAEHQRTHTGEKPYKCPECGKSFSTTGALTEHQRTHTGKKTS

SEQ ID NO: 702 - protein, 215 aa, synthetic construct
  REGION 1..215: note = source = /note="Description of Artificial Sequence: Syntheticpolypeptide"
LEPGEKPYKCPECGKSFSRSDHLTNHQRTHTGEKPYKCPECGKSFSRSDNLVRHQRTHTGEKPYKCPECGKSFSTSGHLVRHQRTHTGEKPYKCPECGKSFSQLAHLRAHQRTHTGEKPYKCPECGKSFSSKKALTEHQRTHTGEKPYKCPECGKSFSSKKALTEHQRTHTPNPHRRTDPSHKPFQYKCPECGKSFSSKKHLAEHQRTHTGKKTS

SEQ ID NO: 703 - protein, 211 aa, synthetic construct
  REGION 1..211: note = source = /note="Description of Artificial Sequence: Syntheticpolypeptide"
LEPGEKPYKCPECGKSFSRSDHLTNHQRTHTGEKPYKCPECGKSFSRSDNLVRHQRTHTGEKPYKCPECGKSFSTSGHLVRHQRTHTGEKPYKCPECGKSFSQLAHLRAHQRTHTGEKPYKCPECGKSFSSKKALTEHQRTHTGEKPYKCPECGKSFSSKKALTEHQRTHTHPRAPIPKPFQYKCPECGKSFSSKKHLAEHQRTHTGKKTS

SEQ ID NO: 704 - protein, 204 aa, synthetic construct
  REGION 1..204: note = source = /note="Description of Artificial Sequence: Syntheticpolypeptide"
LEPGEKPYKCPECGKSFSRSDHLTNHQRTHTGEKPYKCPECGKSFSRSDNLVRHQRTHTGEKPYKCPECGKSFSTSGHLVRHQRTHTGEKPYKCPECGKSFSQLAHLRAHQRTHTGEKPYKCPECGKSFSSKKALTEHQRTHTGEKPYKCPECGKSFSSKKALTEHQRTHTGEKPYKCPECGKSFSTKNSLTEHQRTHTGKKTS

SEQ ID NO: 705 - protein, 215 aa, synthetic construct
  REGION 1..215: note = source = /note="Description of Artificial Sequence: Syntheticpolypeptide"
LEPGEKPYKCPECGKSFSQSGNLTEHQRTHTPNPHRRTDPSHKPFQYKCPECGKSFSTSGHLVRHQRTHTGEKPYKCPECGKSFSQLAHLRAHQRTHTGEKPYKCPECGKSFSRSDELVRHQRTHTGEKPYKCPECGKSFSRSDNLVRHQRTHTGEKPYKCPECGKSFSSPADLTRHQRTHTGEKPYKCPECGKSFSDKKDLTRHQRTHTGKKTS

SEQ ID NO: 706 - protein, 215 aa, synthetic construct
  REGION 1..215: note = source = /note="Description of Artificial Sequence: Syntheticpolypeptide"
LEPGEKPYKCPECGKSFSQAGHLASHQRTHTGEKPYKCPECGKSFSQSGNLTEHQRTHTPNPHRRTDPSHKPFQYKCPECGKSFSTSGHLVRHQRTHTGEKPYKCPECGKSFSQLAHLRAHQRTHTGEKPYKCPECGKSFSRSDELVRHQRTHTGEKPYKCPECGKSFSRSDNLVRHQRTHTGEKPYKCPECGKSFSSPADLTRHQRTHTGKKTS

SEQ ID NO: 707 - protein, 215 aa, synthetic construct
  REGION 1..215: note = source = /note="Description of Artificial Sequence: Syntheticpolypeptide"
LEPGEKPYKCPECGKSFSTSGNLVRHQRTHTGEKPYKCPECGKSFSQAGHLASHQRTHTGEKPYKCPECGKSFSQSGNLTEHQRTHTPNPHRRTDPSHKPFQYKCPECGKSFSTSGHLVRHQRTHTGEKPYKCPECGKSFSQLAHLRAHQRTHTGEKPYKCPECGKSFSRSDELVRHQRTHTGEKPYKCPECGKSFSRSDNLVRHQRTHTGKKTS

SEQ ID NO: 708 - protein, 215 aa, synthetic construct
  REGION 1..215: note = source = /note="Description of Artificial Sequence: Syntheticpolypeptide"
LEPGEKPYKCPECGKSFSTTGNLTVHQRTHTGEKPYKCPECGKSFSTSGNLVRHQRTHTGEKPYKCPECGKSFSQAGHLASHQRTHTGEKPYKCPECGKSFSQSGNLTEHQRTHTPNPHRRTDPSHKPFQYKCPECGKSFSTSGHLVRHQRTHTGEKPYKCPECGKSFSQLAHLRAHQRTHTGEKPYKCPECGKSFSRSDELVRHQRTHTGKKTS

SEQ ID NO: 709 - protein, 215 aa, synthetic construct
  REGION 1..215: note = source = /note="Description of Artificial Sequence: Syntheticpolypeptide"
LEPGEKPYKCPECGKSFSRSDHLTNHQRTHTGEKPYKCPECGKSFSTTGNLTVHQRTHTGEKPYKCPECGKSFSTSGNLVRHQRTHTGEKPYKCPECGKSFSQAGHLASHQRTHTGEKPYKCPECGKSFSQSGNLTEHQRTHTPNPHRRTDPSHKPFQYKCPECGKSFSTSGHLVRHQRTHTGEKPYKCPECGKSFSQLAHLRAHQRTHTGKKTS

SEQ ID NO: 710 - protein, 215 aa, synthetic construct
  REGION 1..215: note = source = /note="Description of Artificial Sequence: Syntheticpolypeptide"
LEPGEKPYKCPECGKSFSTSGELVRHQRTHTGEKPYKCPECGKSFSRSDHLTNHQRTHTGEKPYKCPECGKSFSTTGNLTVHQRTHTGEKPYKCPECGKSFSTSGNLVRHQRTHTGEKPYKCPECGKSFSQAGHLASHQRTHTGEKPYKCPECGKSFSQSGNLTEHQRTHTPNPHRRTDPSHKPFQYKCPECGKSFSTSGHLVRHQRTHTGKKTS

SEQ ID NO: 711 - protein, 211 aa, synthetic construct
  REGION 1..211: note = source = /note="Description of Artificial Sequence: Syntheticpolypeptide"
LEPGEKPYKCPECGKSFSQSGNLTEHQRTHTHPRAPIPKPFQYKCPECGKSFSQSSSLVRHQRTHTGEKPYKCPECGKSFSRSDNLVRHQRTHTGEKPYKCPECGKSFSQAGHLASHQRTHTGEKPYKCPECGKSFSRSDHLTNHQRTHTGEKPYKCPECGKSFSRADNLTEHQRTHTGEKPYKCPECGKSFSTSHSLTEHQRTHTGKKTS

SEQ ID NO: 712 - protein, 211 aa, synthetic construct
  REGION 1..211: note = source = /note="Description of Artificial Sequence: Syntheticpolypeptide"
LEPGEKPYKCPECGKSFSQAGHLASHQRTHTGEKPYKCPECGKSFSQSGNLTEHQRTHTHPRAPIPKPFQYKCPECGKSFSQSSSLVRHQRTHTGEKPYKCPECGKSFSRSDNLVRHQRTHTGEKPYKCPECGKSFSQAGHLASHQRTHTGEKPYKCPECGKSFSRSDHLTNHQRTHTGEKPYKCPECGKSFSRADNLTEHQRTHTGKKTS

SEQ ID NO: 713 - protein, 211 aa, synthetic construct
  REGION 1..211: note = source = /note="Description of Artificial Sequence: Syntheticpolypeptide"
LEPGEKPYKCPECGKSFSTSGNLVRHQRTHTGEKPYKCPECGKSFSQAGHLASHQRTHTGEKPYKCPECGKSFSQSGNLTEHQRTHTHPRAPIPKPFQYKCPECGKSFSQSSSLVRHQRTHTGEKPYKCPECGKSFSRSDNLVRHQRTHTGEKPYKCPECGKSFSQAGHLASHQRTHTGEKPYKCPECGKSFSRSDHLTNHQRTHTGKKTS

SEQ ID NO: 714 - protein, 211 aa, synthetic construct
  REGION 1..211: note = source = /note="Description of Artificial Sequence: Syntheticpolypeptide"
LEPGEKPYKCPECGKSFSTTGNLTVHQRTHTGEKPYKCPECGKSFSTSGNLVRHQRTHTGEKPYKCPECGKSFSQAGHLASHQRTHTGEKPYKCPECGKSFSQSGNLTEHQRTHTHPRAPIPKPFQYKCPECGKSFSQSSSLVRHQRTHTGEKPYKCPECGKSFSRSDNLVRHQRTHTGEKPYKCPECGKSFSQAGHLASHQRTHTGKKTS

SEQ ID NO: 715 - protein, 211 aa, synthetic construct
  REGION 1..211: note = source = /note="Description of Artificial Sequence: Syntheticpolypeptide"
LEPGEKPYKCPECGKSFSRSDHLTNHQRTHTGEKPYKCPECGKSFSTTGNLTVHQRTHTGEKPYKCPECGKSFSTSGNLVRHQRTHTGEKPYKCPECGKSFSQAGHLASHQRTHTGEKPYKCPECGKSFSQSGNLTEHQRTHTHPRAPIPKPFQYKCPECGKSFSQSSSLVRHQRTHTGEKPYKCPECGKSFSRSDNLVRHQRTHTGKKTS

SEQ ID NO: 716 - protein, 211 aa, synthetic construct
  REGION 1..211: note = source = /note="Description of Artificial Sequence: Syntheticpolypeptide"
LEPGEKPYKCPECGKSFSTSGELVRHQRTHTGEKPYKCPECGKSFSRSDHLTNHQRTHTGEKPYKCPECGKSFSTTGNLTVHQRTHTGEKPYKCPECGKSFSTSGNLVRHQRTHTGEKPYKCPECGKSFSQAGHLASHQRTHTGEKPYKCPECGKSFSQSGNLTEHQRTHTHPRAPIPKPFQYKCPECGKSFSQSSSLVRHQRTHTGKKTS

SEQ ID NO: 717 - protein, 204 aa, synthetic construct
  REGION 1..204: note = source = /note="Description of Artificial Sequence: Syntheticpolypeptide"
LEPGEKPYKCPECGKSFSTSGNLTEHQRTHTGEKPYKCPECGKSFSTSGELVRHQRTHTGEKPYKCPECGKSFSRSDHLTNHQRTHTGEKPYKCPECGKSFSTTGNLTVHQRTHTGEKPYKCPECGKSFSTSGNLVRHQRTHTGEKPYKCPECGKSFSQAGHLASHQRTHTGEKPYKCPECGKSFSQSGNLTEHQRTHTGKKTS

SEQ ID NO: 718 - protein, 204 aa, synthetic construct
  REGION 1..204: note = source = /note="Description of Artificial Sequence: Syntheticpolypeptide"
LEPGEKPYKCPECGKSFSRSDNLVRHQRTHTGEKPYKCPECGKSFSTSGNLTEHQRTHTGEKPYKCPECGKSFSTSGELVRHQRTHTGEKPYKCPECGKSFSRSDHLTNHQRTHTGEKPYKCPECGKSFSTTGNLTVHQRTHTGEKPYKCPECGKSFSTSGNLVRHQRTHTGEKPYKCPECGKSFSQAGHLASHQRTHTGKKTS

SEQ ID NO: 719 - protein, 204 aa, synthetic construct
  REGION 1..204: note = source = /note="Description of Artificial Sequence: Syntheticpolypeptide"
LEPGEKPYKCPECGKSFSQRANLRAHQRTHTGEKPYKCPECGKSFSRSDNLVRHQRTHTGEKPYKCPECGKSFSTSGNLTEHQRTHTGEKPYKCPECGKSFSTSGELVRHQRTHTGEKPYKCPECGKSFSRSDHLTNHQRTHTGEKPYKCPECGKSFSTTGNLTVHQRTHTGEKPYKCPECGKSFSTSGNLVRHQRTHTGKKTS

SEQ ID NO: 720 - protein, 204 aa, synthetic construct
  REGION 1..204: note = source = /note="Description of Artificial Sequence: Syntheticpolypeptide"
LEPGEKPYKCPECGKSFSTSHSLTEHQRTHTGEKPYKCPECGKSFSQRANLRAHQRTHTGEKPYKCPECGKSFSRSDNLVRHQRTHTGEKPYKCPECGKSFSTSGNLTEHQRTHTGEKPYKCPECGKSFSTSGELVRHQRTHTGEKPYKCPECGKSFSRSDHLTNHQRTHTGEKPYKCPECGKSFSTTGNLTVHQRTHTGKKTS

SEQ ID NO: 721 - protein, 204 aa, synthetic construct
  REGION 1..204: note = source = /note="Description of Artificial Sequence: Syntheticpolypeptide"
LEPGEKPYKCPECGKSFSRADNLTEHQRTHTGEKPYKCPECGKSFSTSHSLTEHQRTHTGEKPYKCPECGKSFSQRANLRAHQRTHTGEKPYKCPECGKSFSRSDNLVRHQRTHTGEKPYKCPECGKSFSTSGNLTEHQRTHTGEKPYKCPECGKSFSTSGELVRHQRTHTGEKPYKCPECGKSFSRSDHLTNHQRTHTGKKTS

SEQ ID NO: 722 - protein, 215 aa, synthetic construct
  REGION 1..215: note = source = /note="Description of Artificial Sequence: Syntheticpolypeptide"
LEPGEKPYKCPECGKSFSRSDNLVRHQRTHTGEKPYKCPECGKSFSTSGNLTEHQRTHTPNPHRRTDPSHKPFQYKCPECGKSFSRSDKLVRHQRTHTGEKPYKCPECGKSFSQKSSLIAHQRTHTGEKPYKCPECGKSFSQKSSLIAHQRTHTGEKPYKCPECGKSFSRSDNLVRHQRTHTGEKPYKCPECGKSFSTTGNLTVHQRTHTGKKTS

SEQ ID NO: 723 - protein, 215 aa, synthetic construct
  REGION 1..215: note = source = /note="Description of Artificial Sequence: Syntheticpolypeptide"
LEPGEKPYKCPECGKSFSQRANLRAHQRTHTGEKPYKCPECGKSFSRSDNLVRHQRTHTGEKPYKCPECGKSFSTSGNLTEHQRTHTPNPHRRTDPSHKPFQYKCPECGKSFSRSDKLVRHQRTHTGEKPYKCPECGKSFSQKSSLIAHQRTHTGEKPYKCPECGKSFSQKSSLIAHQRTHTGEKPYKCPECGKSFSRSDNLVRHQRTHTGKKTS

SEQ ID NO: 724 - protein, 215 aa, synthetic construct
  REGION 1..215: note = source = /note="Description of Artificial Sequence: Syntheticpolypeptide"
LEPGEKPYKCPECGKSFSTSHSLTEHQRTHTGEKPYKCPECGKSFSQRANLRAHQRTHTGEKPYKCPECGKSFSRSDNLVRHQRTHTGEKPYKCPECGKSFSTSGNLTEHQRTHTPNPHRRTDPSHKPFQYKCPECGKSFSRSDKLVRHQRTHTGEKPYKCPECGKSFSQKSSLIAHQRTHTGEKPYKCPECGKSFSQKSSLIAHQRTHTGKKTS

SEQ ID NO: 725 - protein, 215 aa, synthetic construct
  REGION 1..215: note = source = /note="Description of Artificial Sequence: Syntheticpolypeptide"
LEPGEKPYKCPECGKSFSRADNLTEHQRTHTGEKPYKCPECGKSFSTSHSLTEHQRTHTGEKPYKCPECGKSFSQRANLRAHQRTHTGEKPYKCPECGKSFSRSDNLVRHQRTHTGEKPYKCPECGKSFSTSGNLTEHQRTHTPNPHRRTDPSHKPFQYKCPECGKSFSRSDKLVRHQRTHTGEKPYKCPECGKSFSQKSSLIAHQRTHTGKKTS

SEQ ID NO: 726 - protein, 211 aa, synthetic construct
  REGION 1..211: note = source = /note="Description of Artificial Sequence: Syntheticpolypeptide"
LEPGEKPYKCPECGKSFSRSDNLVRHQRTHTGEKPYKCPECGKSFSTSGNLTEHQRTHTGEKPYKCPECGKSFSTSGELVRHQRTHTGEKPYKCPECGKSFSRSDHLTNHQRTHTGEKPYKCPECGKSFSTTGNLTVHQRTHTHPRAPIPKPFQYKCPECGKSFSQLAHLRAHQRTHTGEKPYKCPECGKSFSRRDELNVHQRTHTGKKTS

SEQ ID NO: 727 - protein, 211 aa, synthetic construct
  REGION 1..211: note = source = /note="Description of Artificial Sequence: Syntheticpolypeptide"
LEPGEKPYKCPECGKSFSQRANLRAHQRTHTGEKPYKCPECGKSFSRSDNLVRHQRTHTGEKPYKCPECGKSFSTSGNLTEHQRTHTGEKPYKCPECGKSFSTSGELVRHQRTHTGEKPYKCPECGKSFSRSDHLTNHQRTHTGEKPYKCPECGKSFSTTGNLTVHQRTHTHPRAPIPKPFQYKCPECGKSFSQLAHLRAHQRTHTGKKTS

SEQ ID NO: 728 - protein, 211 aa, synthetic construct
  REGION 1..211: note = source = /note="Description of Artificial Sequence: Syntheticpolypeptide"
LEPGEKPYKCPECGKSFSRADNLTEHQRTHTGEKPYKCPECGKSFSTSHSLTEHQRTHTGEKPYKCPECGKSFSQRANLRAHQRTHTGEKPYKCPECGKSFSRSDNLVRHQRTHTGEKPYKCPECGKSFSTSGNLTEHQRTHTHPRAPIPKPFQYKCPECGKSFSDPGHLVRHQRTHTGEKPYKCPECGKSFSREDNLHTHQRTHTGKKTS

SEQ ID NO: 729 - protein, 222 aa, synthetic construct
  REGION 1..222: note = source = /note="Description of Artificial Sequence: Syntheticpolypeptide"
LEPGEKPYKCPECGKSFSQAGHLASHQRTHTPNPHRRTDPSHKPFQYKCPECGKSFSRSDNLVRHQRTHTGEKPYKCPECGKSFSQKSSLIAHQRTHTHPRAPIPKPFQYKCPECGKSFSRSDELVRHQRTHTGEKPYKCPECGKSFSQRANLRAHQRTHTGEKPYKCPECGKSFSSPADLTRHQRTHTGEKPYKCPECGKSFSQRANLRAHQRTHTGKKTS

SEQ ID NO: 730 - protein, 222 aa, synthetic construct
  REGION 1..222: note = source = /note="Description of Artificial Sequence: Syntheticpolypeptide"
LEPGEKPYKCPECGKSFSQRANLRAHQRTHTGEKPYKCPECGKSFSQAGHLASHQRTHTPNPHRRTDPSHKPFQYKCPECGKSFSRSDNLVRHQRTHTGEKPYKCPECGKSFSQKSSLIAHQRTHTHPRAPIPKPFQYKCPECGKSFSRSDELVRHQRTHTGEKPYKCPECGKSFSQRANLRAHQRTHTGEKPYKCPECGKSFSSPADLTRHQRTHTGKKTS

SEQ ID NO: 731 - protein, 222 aa, synthetic construct
  REGION 1..222: note = source = /note="Description of Artificial Sequence: Syntheticpolypeptide"
LEPGEKPYKCPECGKSFSQRANLRAHQRTHTGEKPYKCPECGKSFSQRANLRAHQRTHTGEKPYKCPECGKSFSQAGHLASHQRTHTPNPHRRTDPSHKPFQYKCPECGKSFSRSDNLVRHQRTHTGEKPYKCPECGKSFSQKSSLIAHQRTHTHPRAPIPKPFQYKCPECGKSFSRSDELVRHQRTHTGEKPYKCPECGKSFSQRANLRAHQRTHTGKKTS

SEQ ID NO: 732 - protein, 222 aa, synthetic construct
  REGION 1..222: note = source = /note="Description of Artificial Sequence: Syntheticpolypeptide"
LEPGEKPYKCPECGKSFSQRANLRAHQRTHTGEKPYKCPECGKSFSQRANLRAHQRTHTGEKPYKCPECGKSFSQRANLRAHQRTHTGEKPYKCPECGKSFSQAGHLASHQRTHTPNPHRRTDPSHKPFQYKCPECGKSFSRSDNLVRHQRTHTGEKPYKCPECGKSFSQKSSLIAHQRTHTHPRAPIPKPFQYKCPECGKSFSRSDELVRHQRTHTGKKTS

SEQ ID NO: 733 - protein, 218 aa, synthetic construct
  REGION 1..218: note = source = /note="Description of Artificial Sequence: Syntheticpolypeptide"
LEPGEKPYKCPECGKSFSQAGHLASHQRTHTHPRAPIPKPFQYKCPECGKSFSHRTTLTNHQRTHTGEKPYKCPECGKSFSREDNLHTHQRTHTGEKPYKCPECGKSFSQSSSLVRHQRTHTHPRAPIPKPFQYKCPECGKSFSRKDNLKNHQRTHTGEKPYKCPECGKSFSQSGNLTEHQRTHTGEKPYKCPECGKSFSQRANLRAHQRTHTGKKTS

SEQ ID NO: 734 - protein, 218 aa, synthetic construct
  REGION 1..218: note = source = /note="Description of Artificial Sequence: Syntheticpolypeptide"
LEPGEKPYKCPECGKSFSQRANLRAHQRTHTGEKPYKCPECGKSFSQAGHLASHQRTHTHPRAPIPKPFQYKCPECGKSFSHRTTLTNHQRTHTGEKPYKCPECGKSFSREDNLHTHQRTHTGEKPYKCPECGKSFSQSSSLVRHQRTHTHPRAPIPKPFQYKCPECGKSFSRKDNLKNHQRTHTGEKPYKCPECGKSFSQSGNLTEHQRTHTGKKTS

SEQ ID NO: 735 - protein, 218 aa, synthetic construct
  REGION 1..218: note = source = /note="Description of Artificial Sequence: Syntheticpolypeptide"
LEPGEKPYKCPECGKSFSQRANLRAHQRTHTGEKPYKCPECGKSFSQRANLRAHQRTHTGEKPYKCPECGKSFSQAGHLASHQRTHTHPRAPIPKPFQYKCPECGKSFSHRTTLTNHQRTHTGEKPYKCPECGKSFSREDNLHTHQRTHTGEKPYKCPECGKSFSQSSSLVRHQRTHTHPRAPIPKPFQYKCPECGKSFSRKDNLKNHQRTHTGKKTS

SEQ ID NO: 736 - protein, 211 aa, synthetic construct
  REGION 1..211: note = source = /note="Description of Artificial Sequence: Syntheticpolypeptide"
LEPGEKPYKCPECGKSFSQAGHLASHQRTHTHPRAPIPKPFQYKCPECGKSFSHRTTLTNHQRTHTGEKPYKCPECGKSFSREDNLHTHQRTHTGEKPYKCPECGKSFSQSSSLVRHQRTHTGEKPYKCPECGKSFSHRTTLTNHQRTHTGEKPYKCPECGKSFSQRANLRAHQRTHTGEKPYKCPECGKSFSDSGNLRVHQRTHTGKKTS

SEQ ID NO: 737 - protein, 211 aa, synthetic construct
  REGION 1..211: note = source = /note="Description of Artificial Sequence: Syntheticpolypeptide"
LEPGEKPYKCPECGKSFSQRANLRAHQRTHTGEKPYKCPECGKSFSQAGHLASHQRTHTHPRAPIPKPFQYKCPECGKSFSHRTTLTNHQRTHTGEKPYKCPECGKSFSREDNLHTHQRTHTGEKPYKCPECGKSFSQSSSLVRHQRTHTGEKPYKCPECGKSFSHRTTLTNHQRTHTGEKPYKCPECGKSFSQRANLRAHQRTHTGKKTS

SEQ ID NO: 738 - protein, 211 aa, synthetic construct
  REGION 1..211: note = source = /note="Description of Artificial Sequence: Syntheticpolypeptide"
LEPGEKPYKCPECGKSFSQRANLRAHQRTHTGEKPYKCPECGKSFSQRANLRAHQRTHTGEKPYKCPECGKSFSQAGHLASHQRTHTHPRAPIPKPFQYKCPECGKSFSHRTTLTNHQRTHTGEKPYKCPECGKSFSREDNLHTHQRTHTGEKPYKCPECGKSFSQSSSLVRHQRTHTGEKPYKCPECGKSFSHRTTLTNHQRTHTGKKTS

SEQ ID NO: 739 - protein, 211 aa, synthetic construct
  REGION 1..211: note = source = /note="Description of Artificial Sequence: Syntheticpolypeptide"
LEPGEKPYKCPECGKSFSQRANLRAHQRTHTGEKPYKCPECGKSFSQRANLRAHQRTHTGEKPYKCPECGKSFSQRANLRAHQRTHTGEKPYKCPECGKSFSQAGHLASHQRTHTHPRAPIPKPFQYKCPECGKSFSHRTTLTNHQRTHTGEKPYKCPECGKSFSREDNLHTHQRTHTGEKPYKCPECGKSFSQSSSLVRHQRTHTGKKTS

SEQ ID NO: 740 - protein, 211 aa, synthetic construct
  REGION 1..211: note = source = /note="Description of Artificial Sequence: Syntheticpolypeptide"
LEPGEKPYKCPECGKSFSDSGNLRVHQRTHTGEKPYKCPECGKSFSQRANLRAHQRTHTGEKPYKCPECGKSFSQRANLRAHQRTHTGEKPYKCPECGKSFSQRANLRAHQRTHTGEKPYKCPECGKSFSQAGHLASHQRTHTHPRAPIPKPFQYKCPECGKSFSHRTTLTNHQRTHTGEKPYKCPECGKSFSREDNLHTHQRTHTGKKTS

SEQ ID NO: 741 - protein, 211 aa, synthetic construct
  REGION 1..211: note = source = /note="Description of Artificial Sequence: Syntheticpolypeptide"
LEPGEKPYKCPECGKSFSRKDNLKNHQRTHTGEKPYKCPECGKSFSDSGNLRVHQRTHTGEKPYKCPECGKSFSQRANLRAHQRTHTGEKPYKCPECGKSFSQRANLRAHQRTHTGEKPYKCPECGKSFSQRANLRAHQRTHTGEKPYKCPECGKSFSQAGHLASHQRTHTHPRAPIPKPFQYKCPECGKSFSHRTTLTNHQRTHTGKKTS

SEQ ID NO: 742 - protein, 215 aa, synthetic construct
  REGION 1..215: note = source = /note="Description of Artificial Sequence: Syntheticpolypeptide"
LEPGEKPYKCPECGKSFSDSGNLRVHQRTHTGEKPYKCPECGKSFSQRANLRAHQRTHTGEKPYKCPECGKSFSQRANLRAHQRTHTGEKPYKCPECGKSFSQRANLRAHQRTHTGEKPYKCPECGKSFSQAGHLASHQRTHTPNPHRRTDPSHKPFQYKCPECGKSFSRSDNLVRHQRTHTGEKPYKCPECGKSFSQKSSLIAHQRTHTGKKTS

SEQ ID NO: 743 - protein, 215 aa, synthetic construct
  REGION 1..215: note = source = /note="Description of Artificial Sequence: Syntheticpolypeptide"
LEPGEKPYKCPECGKSFSRKDNLKNHQRTHTGEKPYKCPECGKSFSDSGNLRVHQRTHTGEKPYKCPECGKSFSQRANLRAHQRTHTGEKPYKCPECGKSFSQRANLRAHQRTHTGEKPYKCPECGKSFSQRANLRAHQRTHTGEKPYKCPECGKSFSQAGHLASHQRTHTPNPHRRTDPSHKPFQYKCPECGKSFSRSDNLVRHQRTHTGKKTS

SEQ ID NO: 744 - protein, 204 aa, synthetic construct
  REGION 1..204: note = source = /note="Description of Artificial Sequence: Syntheticpolypeptide"
LEPGEKPYKCPECGKSFSDSGNLRVHQRTHTGEKPYKCPECGKSFSQRANLRAHQRTHTGEKPYKCPECGKSFSQRANLRAHQRTHTGEKPYKCPECGKSFSQRANLRAHQRTHTGEKPYKCPECGKSFSQAGHLASHQRTHTGEKPYKCPECGKSFSTSGSLVRHQRTHTGEKPYKCPECGKSFSQLAHLRAHQRTHTGKKTS

SEQ ID NO: 745 - protein, 204 aa, synthetic construct
  REGION 1..204: note = source = /note="Description of Artificial Sequence: Syntheticpolypeptide"
LEPGEKPYKCPECGKSFSRKDNLKNHQRTHTGEKPYKCPECGKSFSDSGNLRVHQRTHTGEKPYKCPECGKSFSQRANLRAHQRTHTGEKPYKCPECGKSFSQRANLRAHQRTHTGEKPYKCPECGKSFSQRANLRAHQRTHTGEKPYKCPECGKSFSQAGHLASHQRTHTGEKPYKCPECGKSFSTSGSLVRHQRTHTGKKTS

SEQ ID NO: 746 - protein, 204 aa, synthetic construct
  REGION 1..204: note = source = /note="Description of Artificial Sequence: Syntheticpolypeptide"
LEPGEKPYKCPECGKSFSQRAHLERHQRTHTGEKPYKCPECGKSFSRKDNLKNHQRTHTGEKPYKCPECGKSFSDSGNLRVHQRTHTGEKPYKCPECGKSFSQRANLRAHQRTHTGEKPYKCPECGKSFSQRANLRAHQRTHTGEKPYKCPECGKSFSQRANLRAHQRTHTGEKPYKCPECGKSFSQAGHLASHQRTHTGKKTS

SEQ ID NO: 747 - protein, 204 aa, synthetic construct
  REGION 1..204: note = source = /note="Description of Artificial Sequence: Syntheticpolypeptide"
LEPGEKPYKCPECGKSFSQRANLRAHQRTHTGEKPYKCPECGKSFSQRAHLERHQRTHTGEKPYKCPECGKSFSRKDNLKNHQRTHTGEKPYKCPECGKSFSDSGNLRVHQRTHTGEKPYKCPECGKSFSQRANLRAHQRTHTGEKPYKCPECGKSFSQRANLRAHQRTHTGEKPYKCPECGKSFSQRANLRAHQRTHTGKKTS

SEQ ID NO: 748 - protein, 204 aa, synthetic construct
  REGION 1..204: note = source = /note="Description of Artificial Sequence: Syntheticpolypeptide"
LEPGEKPYKCPECGKSFSQSSNLVRHQRTHTGEKPYKCPECGKSFSQRANLRAHQRTHTGEKPYKCPECGKSFSQRAHLERHQRTHTGEKPYKCPECGKSFSRKDNLKNHQRTHTGEKPYKCPECGKSFSDSGNLRVHQRTHTGEKPYKCPECGKSFSQRANLRAHQRTHTGEKPYKCPECGKSFSQRANLRAHQRTHTGKKTS

SEQ ID NO: 749 - protein, 204 aa, synthetic construct
  REGION 1..204: note = source = /note="Description of Artificial Sequence: Syntheticpolypeptide"
LEPGEKPYKCPECGKSFSQNSTLTEHQRTHTGEKPYKCPECGKSFSQSSNLVRHQRTHTGEKPYKCPECGKSFSQRANLRAHQRTHTGEKPYKCPECGKSFSQRAHLERHQRTHTGEKPYKCPECGKSFSRKDNLKNHQRTHTGEKPYKCPECGKSFSDSGNLRVHQRTHTGEKPYKCPECGKSFSQRANLRAHQRTHTGKKTS

SEQ ID NO: 750 - protein, 204 aa, synthetic construct
  REGION 1..204: note = source = /note="Description of Artificial Sequence: Syntheticpolypeptide"
LEPGEKPYKCPECGKSFSQRANLRAHQRTHTGEKPYKCPECGKSFSQNSTLTEHQRTHTGEKPYKCPECGKSFSQSSNLVRHQRTHTGEKPYKCPECGKSFSQRANLRAHQRTHTGEKPYKCPECGKSFSQRAHLERHQRTHTGEKPYKCPECGKSFSRKDNLKNHQRTHTGEKPYKCPECGKSFSDSGNLRVHQRTHTGKKTS

SEQ ID NO: 751 - protein, 211 aa, synthetic construct
  REGION 1..211: note = source = /note="Description of Artificial Sequence: Syntheticpolypeptide"
LEPGEKPYKCPECGKSFSQRANLRAHQRTHTHPRAPIPKPFQYKCPECGKSFSQRANLRAHQRTHTGEKPYKCPECGKSFSQNSTLTEHQRTHTGEKPYKCPECGKSFSQSSNLVRHQRTHTGEKPYKCPECGKSFSQRANLRAHQRTHTGEKPYKCPECGKSFSQRAHLERHQRTHTGEKPYKCPECGKSFSRKDNLKNHQRTHTGKKTS

SEQ ID NO: 752 - protein, 218 aa, synthetic construct
  REGION 1..218: note = source = /note="Description of Artificial Sequence: Syntheticpolypeptide"
LEPGEKPYKCPECGKSFSSKKALTEHQRTHTHPRAPIPKPFQYKCPECGKSFSQRANLRAHQRTHTHPRAPIPKPFQYKCPECGKSFSQRANLRAHQRTHTGEKPYKCPECGKSFSQNSTLTEHQRTHTGEKPYKCPECGKSFSQSSNLVRHQRTHTGEKPYKCPECGKSFSQRANLRAHQRTHTGEKPYKCPECGKSFSQRAHLERHQRTHTGKKTS

SEQ ID NO: 753 - protein, 218 aa, synthetic construct
  REGION 1..218: note = source = /note="Description of Artificial Sequence: Syntheticpolypeptide"
LEPGEKPYKCPECGKSFSRSDHLTNHQRTHTGEKPYKCPECGKSFSSKKALTEHQRTHTHPRAPIPKPFQYKCPECGKSFSQRANLRAHQRTHTHPRAPIPKPFQYKCPECGKSFSQRANLRAHQRTHTGEKPYKCPECGKSFSQNSTLTEHQRTHTGEKPYKCPECGKSFSQSSNLVRHQRTHTGEKPYKCPECGKSFSQRANLRAHQRTHTGKKTS

SEQ ID NO: 754 - protein, 218 aa, synthetic construct
  REGION 1..218: note = source = /note="Description of Artificial Sequence: Syntheticpolypeptide"
LEPGEKPYKCPECGKSFSDCRDLARHQRTHTGEKPYKCPECGKSFSRSDHLTNHQRTHTGEKPYKCPECGKSFSSKKALTEHQRTHTHPRAPIPKPFQYKCPECGKSFSQRANLRAHQRTHTHPRAPIPKPFQYKCPECGKSFSQRANLRAHQRTHTGEKPYKCPECGKSFSQNSTLTEHQRTHTGEKPYKCPECGKSFSQSSNLVRHQRTHTGKKTS

SEQ ID NO: 755 - protein, 218 aa, synthetic construct
  REGION 1..218: note = source = /note="Description of Artificial Sequence: Syntheticpolypeptide"
LEPGEKPYKCPECGKSFSDSGNLRVHQRTHTGEKPYKCPECGKSFSDCRDLARHQRTHTGEKPYKCPECGKSFSRSDHLTNHQRTHTGEKPYKCPECGKSFSSKKALTEHQRTHTHPRAPIPKPFQYKCPECGKSFSQRANLRAHQRTHTHPRAPIPKPFQYKCPECGKSFSQRANLRAHQRTHTGEKPYKCPECGKSFSQNSTLTEHQRTHTGKKTS

SEQ ID NO: 756 - protein, 218 aa, synthetic construct
  REGION 1..218: note = source = /note="Description of Artificial Sequence: Syntheticpolypeptide"
LEPGEKPYKCPECGKSFSRNDTLTEHQRTHTGEKPYKCPECGKSFSDSGNLRVHQRTHTGEKPYKCPECGKSFSDCRDLARHQRTHTGEKPYKCPECGKSFSRSDHLTNHQRTHTGEKPYKCPECGKSFSSKKALTEHQRTHTHPRAPIPKPFQYKCPECGKSFSQRANLRAHQRTHTHPRAPIPKPFQYKCPECGKSFSQRANLRAHQRTHTGKKTS

SEQ ID NO: 757 - protein, 204 aa, synthetic construct
  REGION 1..204: note = source = /note="Description of Artificial Sequence: Syntheticpolypeptide"
LEPGEKPYKCPECGKSFSQSSNLVRHQRTHTGEKPYKCPECGKSFSRSDHLTNHQRTHTGEKPYKCPECGKSFSSPADLTRHQRTHTGEKPYKCPECGKSFSQRANLRAHQRTHTGEKPYKCPECGKSFSQRANLRAHQRTHTGEKPYKCPECGKSFSQRANLRAHQRTHTGEKPYKCPECGKSFSQSSNLVRHQRTHTGKKTS

SEQ ID NO: 758 - protein, 204 aa, synthetic construct
  REGION 1..204: note = source = /note="Description of Artificial Sequence: Syntheticpolypeptide"
LEPGEKPYKCPECGKSFSRKDNLKNHQRTHTGEKPYKCPECGKSFSQSSNLVRHQRTHTGEKPYKCPECGKSFSRSDHLTNHQRTHTGEKPYKCPECGKSFSSPADLTRHQRTHTGEKPYKCPECGKSFSQRANLRAHQRTHTGEKPYKCPECGKSFSQRANLRAHQRTHTGEKPYKCPECGKSFSQRANLRAHQRTHTGKKTS

SEQ ID NO: 759 - protein, 204 aa, synthetic construct
  REGION 1..204: note = source = /note="Description of Artificial Sequence: Syntheticpolypeptide"
LEPGEKPYKCPECGKSFSDSGNLRVHQRTHTGEKPYKCPECGKSFSRKDNLKNHQRTHTGEKPYKCPECGKSFSQSSNLVRHQRTHTGEKPYKCPECGKSFSRSDHLTNHQRTHTGEKPYKCPECGKSFSSPADLTRHQRTHTGEKPYKCPECGKSFSQRANLRAHQRTHTGEKPYKCPECGKSFSQRANLRAHQRTHTGKKTS

SEQ ID NO: 760 - protein, 211 aa, synthetic construct
  REGION 1..211: note = source = /note="Description of Artificial Sequence: Syntheticpolypeptide"
LEPGEKPYKCPECGKSFSQRANLRAHQRTHTHPRAPIPKPFQYKCPECGKSFSDSGNLRVHQRTHTGEKPYKCPECGKSFSRKDNLKNHQRTHTGEKPYKCPECGKSFSQSSNLVRHQRTHTGEKPYKCPECGKSFSRSDHLTNHQRTHTGEKPYKCPECGKSFSSPADLTRHQRTHTGEKPYKCPECGKSFSQRANLRAHQRTHTGKKTS

SEQ ID NO: 761 - protein, 211 aa, synthetic construct
  REGION 1..211: note = source = /note="Description of Artificial Sequence: Syntheticpolypeptide"
LEPGEKPYKCPECGKSFSQKSSLIAHQRTHTGEKPYKCPECGKSFSQRANLRAHQRTHTHPRAPIPKPFQYKCPECGKSFSDSGNLRVHQRTHTGEKPYKCPECGKSFSRKDNLKNHQRTHTGEKPYKCPECGKSFSQSSNLVRHQRTHTGEKPYKCPECGKSFSRSDHLTNHQRTHTGEKPYKCPECGKSFSSPADLTRHQRTHTGKKTS

SEQ ID NO: 762 - protein, 211 aa, synthetic construct
  REGION 1..211: note = source = /note="Description of Artificial Sequence: Syntheticpolypeptide"
LEPGEKPYKCPECGKSFSTTGNLTVHQRTHTGEKPYKCPECGKSFSQKSSLIAHQRTHTGEKPYKCPECGKSFSQRANLRAHQRTHTHPRAPIPKPFQYKCPECGKSFSDSGNLRVHQRTHTGEKPYKCPECGKSFSRKDNLKNHQRTHTGEKPYKCPECGKSFSQSSNLVRHQRTHTGEKPYKCPECGKSFSRSDHLTNHQRTHTGKKTS

SEQ ID NO: 763 - protein, 211 aa, synthetic construct
  REGION 1..211: note = source = /note="Description of Artificial Sequence: Syntheticpolypeptide"
LEPGEKPYKCPECGKSFSSKKALTEHQRTHTGEKPYKCPECGKSFSTTGNLTVHQRTHTGEKPYKCPECGKSFSQKSSLIAHQRTHTGEKPYKCPECGKSFSQRANLRAHQRTHTHPRAPIPKPFQYKCPECGKSFSDSGNLRVHQRTHTGEKPYKCPECGKSFSRKDNLKNHQRTHTGEKPYKCPECGKSFSQSSNLVRHQRTHTGKKTS

SEQ ID NO: 764 - protein, 211 aa, synthetic construct
  REGION 1..211: note = source = /note="Description of Artificial Sequence: Syntheticpolypeptide"
LEPGEKPYKCPECGKSFSRSDHLTNHQRTHTGEKPYKCPECGKSFSSKKALTEHQRTHTGEKPYKCPECGKSFSTTGNLTVHQRTHTGEKPYKCPECGKSFSQKSSLIAHQRTHTGEKPYKCPECGKSFSQRANLRAHQRTHTHPRAPIPKPFQYKCPECGKSFSDSGNLRVHQRTHTGEKPYKCPECGKSFSRKDNLKNHQRTHTGKKTS

SEQ ID NO: 765 - protein, 211 aa, synthetic construct
  REGION 1..211: note = source = /note="Description of Artificial Sequence: Syntheticpolypeptide"
LEPGEKPYKCPECGKSFSDCRDLARHQRTHTGEKPYKCPECGKSFSRSDHLTNHQRTHTGEKPYKCPECGKSFSSKKALTEHQRTHTGEKPYKCPECGKSFSTTGNLTVHQRTHTGEKPYKCPECGKSFSQKSSLIAHQRTHTGEKPYKCPECGKSFSQRANLRAHQRTHTHPRAPIPKPFQYKCPECGKSFSDSGNLRVHQRTHTGKKTS

SEQ ID NO: 766 - protein, 204 aa, synthetic construct
  REGION 1..204: note = source = /note="Description of Artificial Sequence: Syntheticpolypeptide"
LEPGEKPYKCPECGKSFSQRANLRAHQRTHTGEKPYKCPECGKSFSRSDKLVRHQRTHTGEKPYKCPECGKSFSQSGNLTEHQRTHTGEKPYKCPECGKSFSQRANLRAHQRTHTGEKPYKCPECGKSFSQRANLRAHQRTHTGEKPYKCPECGKSFSQRANLRAHQRTHTGEKPYKCPECGKSFSQRANLRAHQRTHTGKKTS

SEQ ID NO: 767 - protein, 204 aa, synthetic construct
  REGION 1..204: note = source = /note="Description of Artificial Sequence: Syntheticpolypeptide"
LEPGEKPYKCPECGKSFSQLAHLRAHQRTHTGEKPYKCPECGKSFSQRANLRAHQRTHTGEKPYKCPECGKSFSRSDKLVRHQRTHTGEKPYKCPECGKSFSQSGNLTEHQRTHTGEKPYKCPECGKSFSQRANLRAHQRTHTGEKPYKCPECGKSFSQRANLRAHQRTHTGEKPYKCPECGKSFSQRANLRAHQRTHTGKKTS

SEQ ID NO: 768 - protein, 204 aa, synthetic construct
  REGION 1..204: note = source = /note="Description of Artificial Sequence: Syntheticpolypeptide"
LEPGEKPYKCPECGKSFSTHLDLIRHQRTHTGEKPYKCPECGKSFSQLAHLRAHQRTHTGEKPYKCPECGKSFSQRANLRAHQRTHTGEKPYKCPECGKSFSRSDKLVRHQRTHTGEKPYKCPECGKSFSQSGNLTEHQRTHTGEKPYKCPECGKSFSQRANLRAHQRTHTGEKPYKCPECGKSFSQRANLRAHQRTHTGKKTS

SEQ ID NO: 769 - protein, 204 aa, synthetic construct
  REGION 1..204: note = source = /note="Description of Artificial Sequence: Syntheticpolypeptide"
LEPGEKPYKCPECGKSFSQRANLRAHQRTHTGEKPYKCPECGKSFSTHLDLIRHQRTHTGEKPYKCPECGKSFSQLAHLRAHQRTHTGEKPYKCPECGKSFSQRANLRAHQRTHTGEKPYKCPECGKSFSRSDKLVRHQRTHTGEKPYKCPECGKSFSQSGNLTEHQRTHTGEKPYKCPECGKSFSQRANLRAHQRTHTGKKTS

SEQ ID NO: 770 - protein, 204 aa, synthetic construct
  REGION 1..204: note = source = /note="Description of Artificial Sequence: Syntheticpolypeptide"
LEPGEKPYKCPECGKSFSQKSSLIAHQRTHTGEKPYKCPECGKSFSQRANLRAHQRTHTGEKPYKCPECGKSFSTHLDLIRHQRTHTGEKPYKCPECGKSFSQLAHLRAHQRTHTGEKPYKCPECGKSFSQRANLRAHQRTHTGEKPYKCPECGKSFSRSDKLVRHQRTHTGEKPYKCPECGKSFSQSGNLTEHQRTHTGKKTS

SEQ ID NO: 771 - protein, 204 aa, synthetic construct
  REGION 1..204: note = source = /note="Description of Artificial Sequence: Syntheticpolypeptide"
LEPGEKPYKCPECGKSFSTTGNLTVHQRTHTGEKPYKCPECGKSFSQKSSLIAHQRTHTGEKPYKCPECGKSFSQRANLRAHQRTHTGEKPYKCPECGKSFSTHLDLIRHQRTHTGEKPYKCPECGKSFSQLAHLRAHQRTHTGEKPYKCPECGKSFSQRANLRAHQRTHTGEKPYKCPECGKSFSRSDKLVRHQRTHTGKKTS

SEQ ID NO: 772 - protein, 204 aa, synthetic construct
  REGION 1..204: note = source = /note="Description of Artificial Sequence: Syntheticpolypeptide"
LEPGEKPYKCPECGKSFSSKKALTEHQRTHTGEKPYKCPECGKSFSTTGNLTVHQRTHTGEKPYKCPECGKSFSQKSSLIAHQRTHTGEKPYKCPECGKSFSQRANLRAHQRTHTGEKPYKCPECGKSFSTHLDLIRHQRTHTGEKPYKCPECGKSFSQLAHLRAHQRTHTGEKPYKCPECGKSFSQRANLRAHQRTHTGKKTS

SEQ ID NO: 773 - protein, 204 aa, synthetic construct
  REGION 1..204: note = source = /note="Description of Artificial Sequence: Syntheticpolypeptide"
LEPGEKPYKCPECGKSFSRSDHLTNHQRTHTGEKPYKCPECGKSFSSKKALTEHQRTHTGEKPYKCPECGKSFSTTGNLTVHQRTHTGEKPYKCPECGKSFSQKSSLIAHQRTHTGEKPYKCPECGKSFSQRANLRAHQRTHTGEKPYKCPECGKSFSTHLDLIRHQRTHTGEKPYKCPECGKSFSQLAHLRAHQRTHTGKKTS

SEQ ID NO: 774 - protein, 204 aa, synthetic construct
  REGION 1..204: note = source = /note="Description of Artificial Sequence: Syntheticpolypeptide"
LEPGEKPYKCPECGKSFSDCRDLARHQRTHTGEKPYKCPECGKSFSRSDHLTNHQRTHTGEKPYKCPECGKSFSSKKALTEHQRTHTGEKPYKCPECGKSFSTTGNLTVHQRTHTGEKPYKCPECGKSFSQKSSLIAHQRTHTGEKPYKCPECGKSFSQRANLRAHQRTHTGEKPYKCPECGKSFSTHLDLIRHQRTHTGKKTS

SEQ ID NO: 775 - protein, 204 aa, synthetic construct
  REGION 1..204: note = source = /note="Description of Artificial Sequence: Syntheticpolypeptide"
LEPGEKPYKCPECGKSFSDSGNLRVHQRTHTGEKPYKCPECGKSFSDCRDLARHQRTHTGEKPYKCPECGKSFSRSDHLTNHQRTHTGEKPYKCPECGKSFSSKKALTEHQRTHTGEKPYKCPECGKSFSTTGNLTVHQRTHTGEKPYKCPECGKSFSQKSSLIAHQRTHTGEKPYKCPECGKSFSQRANLRAHQRTHTGKKTS

SEQ ID NO: 776 - protein, 204 aa, synthetic construct
  REGION 1..204: note = source = /note="Description of Artificial Sequence: Syntheticpolypeptide"
LEPGEKPYKCPECGKSFSRNDTLTEHQRTHTGEKPYKCPECGKSFSDSGNLRVHQRTHTGEKPYKCPECGKSFSDCRDLARHQRTHTGEKPYKCPECGKSFSRSDHLTNHQRTHTGEKPYKCPECGKSFSSKKALTEHQRTHTGEKPYKCPECGKSFSTTGNLTVHQRTHTGEKPYKCPECGKSFSQKSSLIAHQRTHTGKKTS

SEQ ID NO: 777 - protein, 204 aa, synthetic construct
  REGION 1..204: note = source = /note="Description of Artificial Sequence: Syntheticpolypeptide"
LEPGEKPYKCPECGKSFSRSDHLTNHQRTHTGEKPYKCPECGKSFSRNDTLTEHQRTHTGEKPYKCPECGKSFSDSGNLRVHQRTHTGEKPYKCPECGKSFSDCRDLARHQRTHTGEKPYKCPECGKSFSRSDHLTNHQRTHTGEKPYKCPECGKSFSSKKALTEHQRTHTGEKPYKCPECGKSFSTTGNLTVHQRTHTGKKTS

SEQ ID NO: 778 - protein, 204 aa, synthetic construct
  REGION 1..204: note = source = /note="Description of Artificial Sequence: Syntheticpolypeptide"
LEPGEKPYKCPECGKSFSRADNLTEHQRTHTGEKPYKCPECGKSFSRSDHLTNHQRTHTGEKPYKCPECGKSFSRNDTLTEHQRTHTGEKPYKCPECGKSFSDSGNLRVHQRTHTGEKPYKCPECGKSFSDCRDLARHQRTHTGEKPYKCPECGKSFSRSDHLTNHQRTHTGEKPYKCPECGKSFSSKKALTEHQRTHTGKKTS

SEQ ID NO: 779 - protein, 204 aa, synthetic construct
  REGION 1..204: note = source = /note="Description of Artificial Sequence: Syntheticpolypeptide"
LEPGEKPYKCPECGKSFSQSGDLRRHQRTHTGEKPYKCPECGKSFSRADNLTEHQRTHTGEKPYKCPECGKSFSRSDHLTNHQRTHTGEKPYKCPECGKSFSRNDTLTEHQRTHTGEKPYKCPECGKSFSDSGNLRVHQRTHTGEKPYKCPECGKSFSDCRDLARHQRTHTGEKPYKCPECGKSFSRSDHLTNHQRTHTGKKTS

SEQ ID NO: 780 - protein, 204 aa, synthetic construct
  REGION 1..204: note = source = /note="Description of Artificial Sequence: Syntheticpolypeptide"
LEPGEKPYKCPECGKSFSRSDNLVRHQRTHTGEKPYKCPECGKSFSQSGDLRRHQRTHTGEKPYKCPECGKSFSRADNLTEHQRTHTGEKPYKCPECGKSFSRSDHLTNHQRTHTGEKPYKCPECGKSFSRNDTLTEHQRTHTGEKPYKCPECGKSFSDSGNLRVHQRTHTGEKPYKCPECGKSFSDCRDLARHQRTHTGKKTS

SEQ ID NO: 781 - protein, 204 aa, synthetic construct
  REGION 1..204: note = source = /note="Description of Artificial Sequence: Syntheticpolypeptide"
LEPGEKPYKCPECGKSFSSPADLTRHQRTHTGEKPYKCPECGKSFSRSDNLVRHQRTHTGEKPYKCPECGKSFSQSGDLRRHQRTHTGEKPYKCPECGKSFSRADNLTEHQRTHTGEKPYKCPECGKSFSRSDHLTNHQRTHTGEKPYKCPECGKSFSRNDTLTEHQRTHTGEKPYKCPECGKSFSDSGNLRVHQRTHTGKKTS

SEQ ID NO: 782 - protein, 204 aa, synthetic construct
  REGION 1..204: note = source = /note="Description of Artificial Sequence: Syntheticpolypeptide"
LEPGEKPYKCPECGKSFSTSHSLTEHQRTHTGEKPYKCPECGKSFSSPADLTRHQRTHTGEKPYKCPECGKSFSRSDNLVRHQRTHTGEKPYKCPECGKSFSQSGDLRRHQRTHTGEKPYKCPECGKSFSRADNLTEHQRTHTGEKPYKCPECGKSFSRSDHLTNHQRTHTGEKPYKCPECGKSFSRNDTLTEHQRTHTGKKTS

SEQ ID NO: 783 - protein, 215 aa, synthetic construct
  REGION 1..215: note = source = /note="Description of Artificial Sequence: Syntheticpolypeptide"
LEPGEKPYKCPECGKSFSRNDALTEHQRTHTPNPHRRTDPSHKPFQYKCPECGKSFSTSHSLTEHQRTHTGEKPYKCPECGKSFSSPADLTRHQRTHTGEKPYKCPECGKSFSRSDNLVRHQRTHTGEKPYKCPECGKSFSQSGDLRRHQRTHTGEKPYKCPECGKSFSRADNLTEHQRTHTGEKPYKCPECGKSFSRSDHLTNHQRTHTGKKTS

SEQ ID NO: 784 - protein, 215 aa, synthetic construct
  REGION 1..215: note = source = /note="Description of Artificial Sequence: Syntheticpolypeptide"
LEPGEKPYKCPECGKSFSQAGHLASHQRTHTGEKPYKCPECGKSFSRNDALTEHQRTHTPNPHRRTDPSHKPFQYKCPECGKSFSTSHSLTEHQRTHTGEKPYKCPECGKSFSSPADLTRHQRTHTGEKPYKCPECGKSFSRSDNLVRHQRTHTGEKPYKCPECGKSFSQSGDLRRHQRTHTGEKPYKCPECGKSFSRADNLTEHQRTHTGKKTS

SEQ ID NO: 785 - protein, 215 aa, synthetic construct
  REGION 1..215: note = source = /note="Description of Artificial Sequence: Syntheticpolypeptide"
LEPGEKPYKCPECGKSFSRSDELVRHQRTHTGEKPYKCPECGKSFSQAGHLASHQRTHTGEKPYKCPECGKSFSRNDALTEHQRTHTPNPHRRTDPSHKPFQYKCPECGKSFSTSHSLTEHQRTHTGEKPYKCPECGKSFSSPADLTRHQRTHTGEKPYKCPECGKSFSRSDNLVRHQRTHTGEKPYKCPECGKSFSQSGDLRRHQRTHTGKKTS

SEQ ID NO: 786 - protein, 215 aa, synthetic construct
  REGION 1..215: note = source = /note="Description of Artificial Sequence: Syntheticpolypeptide"
LEPGEKPYKCPECGKSFSQLAHLRAHQRTHTGEKPYKCPECGKSFSRSDELVRHQRTHTGEKPYKCPECGKSFSQAGHLASHQRTHTGEKPYKCPECGKSFSRNDALTEHQRTHTPNPHRRTDPSHKPFQYKCPECGKSFSTSHSLTEHQRTHTGEKPYKCPECGKSFSSPADLTRHQRTHTGEKPYKCPECGKSFSRSDNLVRHQRTHTGKKTS

SEQ ID NO: 787 - protein, 215 aa, synthetic construct
  REGION 1..215: note = source = /note="Description of Artificial Sequence: Syntheticpolypeptide"
LEPGEKPYKCPECGKSFSQRAHLERHQRTHTGEKPYKCPECGKSFSQLAHLRAHQRTHTGEKPYKCPECGKSFSRSDELVRHQRTHTGEKPYKCPECGKSFSQAGHLASHQRTHTGEKPYKCPECGKSFSRNDALTEHQRTHTPNPHRRTDPSHKPFQYKCPECGKSFSTSHSLTEHQRTHTGEKPYKCPECGKSFSSPADLTRHQRTHTGKKTS

SEQ ID NO: 788 - protein, 222 aa, synthetic construct
  REGION 1..222: note = source = /note="Description of Artificial Sequence: Syntheticpolypeptide"
LEPGEKPYKCPECGKSFSHKNALQNHQRTHTHPRAPIPKPFQYKCPECGKSFSQRAHLERHQRTHTGEKPYKCPECGKSFSQLAHLRAHQRTHTGEKPYKCPECGKSFSRSDELVRHQRTHTGEKPYKCPECGKSFSQAGHLASHQRTHTGEKPYKCPECGKSFSRNDALTEHQRTHTPNPHRRTDPSHKPFQYKCPECGKSFSTSHSLTEHQRTHTGKKTS

SEQ ID NO: 789 - protein, 211 aa, synthetic construct
  REGION 1..211: note = source = /note="Description of Artificial Sequence: Syntheticpolypeptide"
LEPGEKPYKCPECGKSFSRNDTLTEHQRTHTGEKPYKCPECGKSFSDSGNLRVHQRTHTGEKPYKCPECGKSFSDCRDLARHQRTHTGEKPYKCPECGKSFSRSDHLTNHQRTHTGEKPYKCPECGKSFSSKKALTEHQRTHTHPRAPIPKPFQYKCPECGKSFSQRANLRAHQRTHTGEKPYKCPECGKSFSTTGNLTVHQRTHTGKKTS

SEQ ID NO: 790 - protein, 211 aa, synthetic construct
  REGION 1..211: note = source = /note="Description of Artificial Sequence: Syntheticpolypeptide"
LEPGEKPYKCPECGKSFSRSDHLTNHQRTHTGEKPYKCPECGKSFSRNDTLTEHQRTHTGEKPYKCPECGKSFSDSGNLRVHQRTHTGEKPYKCPECGKSFSDCRDLARHQRTHTGEKPYKCPECGKSFSRSDHLTNHQRTHTGEKPYKCPECGKSFSSKKALTEHQRTHTHPRAPIPKPFQYKCPECGKSFSQRANLRAHQRTHTGKKTS

SEQ ID NO: 791 - protein, 204 aa, synthetic construct
  REGION 1..204: note = source = /note="Description of Artificial Sequence: Syntheticpolypeptide"
LEPGEKPYKCPECGKSFSRSDHLTNHQRTHTGEKPYKCPECGKSFSDPGHLVRHQRTHTGEKPYKCPECGKSFSQSGHLTEHQRTHTGEKPYKCPECGKSFSDKKDLTRHQRTHTGEKPYKCPECGKSFSTSHSLTEHQRTHTGEKPYKCPECGKSFSRSDKLVRHQRTHTGEKPYKCPECGKSFSSPADLTRHQRTHTGKKTS

SEQ ID NO: 792 - protein, 204 aa, synthetic construct
  REGION 1..204: note = source = /note="Description of Artificial Sequence: Syntheticpolypeptide"
LEPGEKPYKCPECGKSFSRADNLTEHQRTHTGEKPYKCPECGKSFSRSDHLTNHQRTHTGEKPYKCPECGKSFSDPGHLVRHQRTHTGEKPYKCPECGKSFSQSGHLTEHQRTHTGEKPYKCPECGKSFSDKKDLTRHQRTHTGEKPYKCPECGKSFSTSHSLTEHQRTHTGEKPYKCPECGKSFSRSDKLVRHQRTHTGKKTS

SEQ ID NO: 793 - protein, 204 aa, synthetic construct
  REGION 1..204: note = source = /note="Description of Artificial Sequence: Syntheticpolypeptide"
LEPGEKPYKCPECGKSFSQLAHLRAHQRTHTGEKPYKCPECGKSFSRADNLTEHQRTHTGEKPYKCPECGKSFSRSDHLTNHQRTHTGEKPYKCPECGKSFSDPGHLVRHQRTHTGEKPYKCPECGKSFSQSGHLTEHQRTHTGEKPYKCPECGKSFSDKKDLTRHQRTHTGEKPYKCPECGKSFSTSHSLTEHQRTHTGKKTS

SEQ ID NO: 794 - protein, 204 aa, synthetic construct
  REGION 1..204: note = source = /note="Description of Artificial Sequence: Syntheticpolypeptide"
LEPGEKPYKCPECGKSFSSKKALTEHQRTHTGEKPYKCPECGKSFSQLAHLRAHQRTHTGEKPYKCPECGKSFSRADNLTEHQRTHTGEKPYKCPECGKSFSRSDHLTNHQRTHTGEKPYKCPECGKSFSDPGHLVRHQRTHTGEKPYKCPECGKSFSQSGHLTEHQRTHTGEKPYKCPECGKSFSDKKDLTRHQRTHTGKKTS

SEQ ID NO: 795 - protein, 204 aa, synthetic construct
  REGION 1..204: note = source = /note="Description of Artificial Sequence: Syntheticpolypeptide"
LEPGEKPYKCPECGKSFSTSGNLTEHQRTHTGEKPYKCPECGKSFSSKKALTEHQRTHTGEKPYKCPECGKSFSQLAHLRAHQRTHTGEKPYKCPECGKSFSRADNLTEHQRTHTGEKPYKCPECGKSFSRSDHLTNHQRTHTGEKPYKCPECGKSFSDPGHLVRHQRTHTGEKPYKCPECGKSFSQSGHLTEHQRTHTGKKTS

SEQ ID NO: 796 - protein, 211 aa, synthetic construct
  REGION 1..211: note = source = /note="Description of Artificial Sequence: Syntheticpolypeptide"
LEPGEKPYKCPECGKSFSRNDALTEHQRTHTHPRAPIPKPFQYKCPECGKSFSTSGNLTEHQRTHTGEKPYKCPECGKSFSSKKALTEHQRTHTGEKPYKCPECGKSFSQLAHLRAHQRTHTGEKPYKCPECGKSFSRADNLTEHQRTHTGEKPYKCPECGKSFSRSDHLTNHQRTHTGEKPYKCPECGKSFSDPGHLVRHQRTHTGKKTS

SEQ ID NO: 797 - protein, 211 aa, synthetic construct
  REGION 1..211: note = source = /note="Description of Artificial Sequence: Syntheticpolypeptide"
LEPGEKPYKCPECGKSFSQAGHLASHQRTHTGEKPYKCPECGKSFSRNDALTEHQRTHTHPRAPIPKPFQYKCPECGKSFSTSGNLTEHQRTHTGEKPYKCPECGKSFSSKKALTEHQRTHTGEKPYKCPECGKSFSQLAHLRAHQRTHTGEKPYKCPECGKSFSRADNLTEHQRTHTGEKPYKCPECGKSFSRSDHLTNHQRTHTGKKTS

SEQ ID NO: 798 - protein, 211 aa, synthetic construct
  REGION 1..211: note = source = /note="Description of Artificial Sequence: Syntheticpolypeptide"
LEPGEKPYKCPECGKSFSRSDELVRHQRTHTGEKPYKCPECGKSFSQAGHLASHQRTHTGEKPYKCPECGKSFSRNDALTEHQRTHTHPRAPIPKPFQYKCPECGKSFSTSGNLTEHQRTHTGEKPYKCPECGKSFSSKKALTEHQRTHTGEKPYKCPECGKSFSQLAHLRAHQRTHTGEKPYKCPECGKSFSRADNLTEHQRTHTGKKTS

SEQ ID NO: 799 - protein, 211 aa, synthetic construct
  REGION 1..211: note = source = /note="Description of Artificial Sequence: Syntheticpolypeptide"
LEPGEKPYKCPECGKSFSQLAHLRAHQRTHTGEKPYKCPECGKSFSRSDELVRHQRTHTGEKPYKCPECGKSFSQAGHLASHQRTHTGEKPYKCPECGKSFSRNDALTEHQRTHTHPRAPIPKPFQYKCPECGKSFSTSGNLTEHQRTHTGEKPYKCPECGKSFSSKKALTEHQRTHTGEKPYKCPECGKSFSQLAHLRAHQRTHTGKKTS

SEQ ID NO: 800 - protein, 211 aa, synthetic construct
  REGION 1..211: note = source = /note="Description of Artificial Sequence: Syntheticpolypeptide"
LEPGEKPYKCPECGKSFSQRAHLERHQRTHTGEKPYKCPECGKSFSQLAHLRAHQRTHTGEKPYKCPECGKSFSRSDELVRHQRTHTGEKPYKCPECGKSFSQAGHLASHQRTHTGEKPYKCPECGKSFSRNDALTEHQRTHTHPRAPIPKPFQYKCPECGKSFSTSGNLTEHQRTHTGEKPYKCPECGKSFSSKKALTEHQRTHTGKKTS

SEQ ID NO: 801 - protein, 218 aa, synthetic construct
  REGION 1..218: note = source = /note="Description of Artificial Sequence: Syntheticpolypeptide"
LEPGEKPYKCPECGKSFSHKNALQNHQRTHTHPRAPIPKPFQYKCPECGKSFSQRAHLERHQRTHTGEKPYKCPECGKSFSQLAHLRAHQRTHTGEKPYKCPECGKSFSRSDELVRHQRTHTGEKPYKCPECGKSFSQAGHLASHQRTHTGEKPYKCPECGKSFSRNDALTEHQRTHTHPRAPIPKPFQYKCPECGKSFSTSGNLTEHQRTHTGKKTS

SEQ ID NO: 802 - protein, 204 aa, synthetic construct
  REGION 1..204: note = source = /note="Description of Artificial Sequence: Syntheticpolypeptide"
LEPGEKPYKCPECGKSFSDPGHLVRHQRTHTGEKPYKCPECGKSFSQSGDLRRHQRTHTGEKPYKCPECGKSFSRSDNLVRHQRTHTGEKPYKCPECGKSFSSKKHLAEHQRTHTGEKPYKCPECGKSFSQSGNLTEHQRTHTGEKPYKCPECGKSFSDPGHLVRHQRTHTGEKPYKCPECGKSFSRADNLTEHQRTHTGKKTS

SEQ ID NO: 803 - protein, 204 aa, synthetic construct
  REGION 1..204: note = source = /note="Description of Artificial Sequence: Syntheticpolypeptide"
LEPGEKPYKCPECGKSFSQLAHLRAHQRTHTGEKPYKCPECGKSFSDPGHLVRHQRTHTGEKPYKCPECGKSFSQSGDLRRHQRTHTGEKPYKCPECGKSFSRSDNLVRHQRTHTGEKPYKCPECGKSFSSKKHLAEHQRTHTGEKPYKCPECGKSFSQSGNLTEHQRTHTGEKPYKCPECGKSFSDPGHLVRHQRTHTGKKTS

SEQ ID NO: 804 - protein, 204 aa, synthetic construct
  REGION 1..204: note = source = /note="Description of Artificial Sequence: Syntheticpolypeptide"
LEPGEKPYKCPECGKSFSDPGNLVRHQRTHTGEKPYKCPECGKSFSQLAHLRAHQRTHTGEKPYKCPECGKSFSDPGHLVRHQRTHTGEKPYKCPECGKSFSQSGDLRRHQRTHTGEKPYKCPECGKSFSRSDNLVRHQRTHTGEKPYKCPECGKSFSSKKHLAEHQRTHTGEKPYKCPECGKSFSQSGNLTEHQRTHTGKKTS

SEQ ID NO: 805 - protein, 204 aa, synthetic construct
  REGION 1..204: note = source = /note="Description of Artificial Sequence: Syntheticpolypeptide"
LEPGEKPYKCPECGKSFSDKKDLTRHQRTHTGEKPYKCPECGKSFSDPGNLVRHQRTHTGEKPYKCPECGKSFSQLAHLRAHQRTHTGEKPYKCPECGKSFSDPGHLVRHQRTHTGEKPYKCPECGKSFSQSGDLRRHQRTHTGEKPYKCPECGKSFSRSDNLVRHQRTHTGEKPYKCPECGKSFSSKKHLAEHQRTHTGKKTS

SEQ ID NO: 806 - protein, 204 aa, synthetic construct
  REGION 1..204: note = source = /note="Description of Artificial Sequence: Syntheticpolypeptide"
LEPGEKPYKCPECGKSFSHKNALQNHQRTHTGEKPYKCPECGKSFSDKKDLTRHQRTHTGEKPYKCPECGKSFSDPGNLVRHQRTHTGEKPYKCPECGKSFSQLAHLRAHQRTHTGEKPYKCPECGKSFSDPGHLVRHQRTHTGEKPYKCPECGKSFSQSGDLRRHQRTHTGEKPYKCPECGKSFSRSDNLVRHQRTHTGKKTS

SEQ ID NO: 807 - protein, 204 aa, synthetic construct
  REGION 1..204: note = source = /note="Description of Artificial Sequence: Syntheticpolypeptide"
LEPGEKPYKCPECGKSFSRNDALTEHQRTHTGEKPYKCPECGKSFSHKNALQNHQRTHTGEKPYKCPECGKSFSDKKDLTRHQRTHTGEKPYKCPECGKSFSDPGNLVRHQRTHTGEKPYKCPECGKSFSQLAHLRAHQRTHTGEKPYKCPECGKSFSDPGHLVRHQRTHTGEKPYKCPECGKSFSQSGDLRRHQRTHTGKKTS

SEQ ID NO: 808 - protein, 204 aa, synthetic construct
  REGION 1..204: note = source = /note="Description of Artificial Sequence: Syntheticpolypeptide"
LEPGEKPYKCPECGKSFSQAGHLASHQRTHTGEKPYKCPECGKSFSRNDALTEHQRTHTGEKPYKCPECGKSFSHKNALQNHQRTHTGEKPYKCPECGKSFSDKKDLTRHQRTHTGEKPYKCPECGKSFSDPGNLVRHQRTHTGEKPYKCPECGKSFSQLAHLRAHQRTHTGEKPYKCPECGKSFSDPGHLVRHQRTHTGKKTS

SEQ ID NO: 809 - protein, 204 aa, synthetic construct
  REGION 1..204: note = source = /note="Description of Artificial Sequence: Syntheticpolypeptide"
LEPGEKPYKCPECGKSFSRSDELVRHQRTHTGEKPYKCPECGKSFSQAGHLASHQRTHTGEKPYKCPECGKSFSRNDALTEHQRTHTGEKPYKCPECGKSFSHKNALQNHQRTHTGEKPYKCPECGKSFSDKKDLTRHQRTHTGEKPYKCPECGKSFSDPGNLVRHQRTHTGEKPYKCPECGKSFSQLAHLRAHQRTHTGKKTS

SEQ ID NO: 810 - protein, 204 aa, synthetic construct
  REGION 1..204: note = source = /note="Description of Artificial Sequence: Syntheticpolypeptide"
LEPGEKPYKCPECGKSFSQLAHLRAHQRTHTGEKPYKCPECGKSFSRSDELVRHQRTHTGEKPYKCPECGKSFSQAGHLASHQRTHTGEKPYKCPECGKSFSRNDALTEHQRTHTGEKPYKCPECGKSFSHKNALQNHQRTHTGEKPYKCPECGKSFSDKKDLTRHQRTHTGEKPYKCPECGKSFSDPGNLVRHQRTHTGKKTS

SEQ ID NO: 811 - protein, 204 aa, synthetic construct
  REGION 1..204: note = source = /note="Description of Artificial Sequence: Syntheticpolypeptide"
LEPGEKPYKCPECGKSFSQRAHLERHQRTHTGEKPYKCPECGKSFSQLAHLRAHQRTHTGEKPYKCPECGKSFSRSDELVRHQRTHTGEKPYKCPECGKSFSQAGHLASHQRTHTGEKPYKCPECGKSFSRNDALTEHQRTHTGEKPYKCPECGKSFSHKNALQNHQRTHTGEKPYKCPECGKSFSDKKDLTRHQRTHTGKKTS

SEQ ID NO: 812 - protein, 211 aa, synthetic construct
  REGION 1..211: note = source = /note="Description of Artificial Sequence: Syntheticpolypeptide"
LEPGEKPYKCPECGKSFSHKNALQNHQRTHTHPRAPIPKPFQYKCPECGKSFSQRAHLERHQRTHTGEKPYKCPECGKSFSQLAHLRAHQRTHTGEKPYKCPECGKSFSRSDELVRHQRTHTGEKPYKCPECGKSFSQAGHLASHQRTHTGEKPYKCPECGKSFSRNDALTEHQRTHTGEKPYKCPECGKSFSHKNALQNHQRTHTGKKTS

SEQ ID NO: 813 - protein, 211 aa, synthetic construct
  REGION 1..211: note = source = /note="Description of Artificial Sequence: Syntheticpolypeptide"
LEPGEKPYKCPECGKSFSQAGHLASHQRTHTGEKPYKCPECGKSFSHKNALQNHQRTHTHPRAPIPKPFQYKCPECGKSFSQRAHLERHQRTHTGEKPYKCPECGKSFSQLAHLRAHQRTHTGEKPYKCPECGKSFSRSDELVRHQRTHTGEKPYKCPECGKSFSQAGHLASHQRTHTGEKPYKCPECGKSFSRNDALTEHQRTHTGKKTS

SEQ ID NO: 814 - protein, 211 aa, synthetic construct
  REGION 1..211: note = source = /note="Description of Artificial Sequence: Syntheticpolypeptide"
LEPGEKPYKCPECGKSFSRSDKLVRHQRTHTGEKPYKCPECGKSFSQAGHLASHQRTHTGEKPYKCPECGKSFSHKNALQNHQRTHTHPRAPIPKPFQYKCPECGKSFSQRAHLERHQRTHTGEKPYKCPECGKSFSQLAHLRAHQRTHTGEKPYKCPECGKSFSRSDELVRHQRTHTGEKPYKCPECGKSFSQAGHLASHQRTHTGKKTS

SEQ ID NO: 815 - protein, 215 aa, synthetic construct
  REGION 1..215: note = source = /note="Description of Artificial Sequence: Syntheticpolypeptide"
LEPGEKPYKCPECGKSFSRSDKLVRHQRTHTGEKPYKCPECGKSFSQAGHLASHQRTHTPNPHRRTDPSHKPFQYKCPECGKSFSQKSSLIAHQRTHTGEKPYKCPECGKSFSRSDHLTNHQRTHTGEKPYKCPECGKSFSRSDNLVRHQRTHTGEKPYKCPECGKSFSHRTTLTNHQRTHTGEKPYKCPECGKSFSRSDELVRHQRTHTGKKTS

SEQ ID NO: 816 - protein, 204 aa, synthetic construct
  REGION 1..204: note = source = /note="Description of Artificial Sequence: Syntheticpolypeptide"
LEPGEKPYKCPECGKSFSRSDKLVRHQRTHTGEKPYKCPECGKSFSQAGHLASHQRTHTGEKPYKCPECGKSFSHKNALQNHQRTHTGEKPYKCPECGKSFSTSGNLVRHQRTHTGEKPYKCPECGKSFSRSDNLVRHQRTHTGEKPYKCPECGKSFSQAGHLASHQRTHTGEKPYKCPECGKSFSRSDNLVRHQRTHTGKKTS

SEQ ID NO: 817 - protein, 215 aa, synthetic construct
  REGION 1..215: note = source = /note="Description of Artificial Sequence: Syntheticpolypeptide"
LEPGEKPYKCPECGKSFSQLAHLRAHQRTHTGEKPYKCPECGKSFSQSSNLVRHQRTHTGEKPYKCPECGKSFSQRANLRAHQRTHTGEKPYKCPECGKSFSRSDKLVRHQRTHTGEKPYKCPECGKSFSRNDALTEHQRTHTPNPHRRTDPSHKPFQYKCPECGKSFSTSGHLVRHQRTHTGEKPYKCPECGKSFSRSDNLVRHQRTHTGKKTS

SEQ ID NO: 818 - protein, 215 aa, synthetic construct
  REGION 1..215: note = source = /note="Description of Artificial Sequence: Syntheticpolypeptide"
LEPGEKPYKCPECGKSFSQSSNLVRHQRTHTGEKPYKCPECGKSFSQLAHLRAHQRTHTGEKPYKCPECGKSFSQSSNLVRHQRTHTGEKPYKCPECGKSFSQRANLRAHQRTHTGEKPYKCPECGKSFSRSDKLVRHQRTHTGEKPYKCPECGKSFSRNDALTEHQRTHTPNPHRRTDPSHKPFQYKCPECGKSFSTSGHLVRHQRTHTGKKTS

SEQ ID NO: 819 - protein, 211 aa, synthetic construct
  REGION 1..211: note = source = /note="Description of Artificial Sequence: Syntheticpolypeptide"
LEPGEKPYKCPECGKSFSQLAHLRAHQRTHTGEKPYKCPECGKSFSQSSNLVRHQRTHTGEKPYKCPECGKSFSQRANLRAHQRTHTGEKPYKCPECGKSFSRSDKLVRHQRTHTGEKPYKCPECGKSFSRNDALTEHQRTHTHPRAPIPKPFQYKCPECGKSFSDPGALVRHQRTHTGEKPYKCPECGKSFSRSDHLTNHQRTHTGKKTS

SEQ ID NO: 820 - protein, 211 aa, synthetic construct
  REGION 1..211: note = source = /note="Description of Artificial Sequence: Syntheticpolypeptide"
LEPGEKPYKCPECGKSFSQSSNLVRHQRTHTGEKPYKCPECGKSFSQLAHLRAHQRTHTGEKPYKCPECGKSFSQSSNLVRHQRTHTGEKPYKCPECGKSFSQRANLRAHQRTHTGEKPYKCPECGKSFSRSDKLVRHQRTHTGEKPYKCPECGKSFSRNDALTEHQRTHTHPRAPIPKPFQYKCPECGKSFSDPGALVRHQRTHTGKKTS

SEQ ID NO: 821 - protein, 211 aa, synthetic construct
  REGION 1..211: note = source = /note="Description of Artificial Sequence: Syntheticpolypeptide"
LEPGEKPYKCPECGKSFSRNDALTEHQRTHTHPRAPIPKPFQYKCPECGKSFSQSSNLVRHQRTHTGEKPYKCPECGKSFSQLAHLRAHQRTHTGEKPYKCPECGKSFSQSSNLVRHQRTHTGEKPYKCPECGKSFSQRANLRAHQRTHTGEKPYKCPECGKSFSRSDKLVRHQRTHTGEKPYKCPECGKSFSRNDALTEHQRTHTGKKTS

SEQ ID NO: 822 - protein, 211 aa, synthetic construct
  REGION 1..211: note = source = /note="Description of Artificial Sequence: Syntheticpolypeptide"
LEPGEKPYKCPECGKSFSRKDNLKNHQRTHTGEKPYKCPECGKSFSRNDALTEHQRTHTHPRAPIPKPFQYKCPECGKSFSQSSNLVRHQRTHTGEKPYKCPECGKSFSQLAHLRAHQRTHTGEKPYKCPECGKSFSQSSNLVRHQRTHTGEKPYKCPECGKSFSQRANLRAHQRTHTGEKPYKCPECGKSFSRSDKLVRHQRTHTGKKTS

SEQ ID NO: 823 - protein, 222 aa, synthetic construct
  REGION 1..222: note = source = /note="Description of Artificial Sequence: Syntheticpolypeptide"
LEPGEKPYKCPECGKSFSQNSTLTEHQRTHTPNPHRRTDPSHKPFQYKCPECGKSFSRKDNLKNHQRTHTGEKPYKCPECGKSFSRNDALTEHQRTHTHPRAPIPKPFQYKCPECGKSFSQSSNLVRHQRTHTGEKPYKCPECGKSFSQLAHLRAHQRTHTGEKPYKCPECGKSFSQSSNLVRHQRTHTGEKPYKCPECGKSFSQRANLRAHQRTHTGKKTS

SEQ ID NO: 824 - protein, 229 aa, synthetic construct
  REGION 1..229: note = source = /note="Description of Artificial Sequence: Syntheticpolypeptide"
LEPGEKPYKCPECGKSFSRSDELVRHQRTHTHPRAPIPKPFQYKCPECGKSFSQNSTLTEHQRTHTPNPHRRTDPSHKPFQYKCPECGKSFSRKDNLKNHQRTHTGEKPYKCPECGKSFSRNDALTEHQRTHTHPRAPIPKPFQYKCPECGKSFSQSSNLVRHQRTHTGEKPYKCPECGKSFSQLAHLRAHQRTHTGEKPYKCPECGKSFSQSSNLVRHQRTHTGKKTS

SEQ ID NO: 825 - protein, 229 aa, synthetic construct
  REGION 1..229: note = source = /note="Description of Artificial Sequence: Syntheticpolypeptide"
LEPGEKPYKCPECGKSFSRSDHLTTHQRTHTGEKPYKCPECGKSFSRSDELVRHQRTHTHPRAPIPKPFQYKCPECGKSFSQNSTLTEHQRTHTPNPHRRTDPSHKPFQYKCPECGKSFSRKDNLKNHQRTHTGEKPYKCPECGKSFSRNDALTEHQRTHTHPRAPIPKPFQYKCPECGKSFSQSSNLVRHQRTHTGEKPYKCPECGKSFSQLAHLRAHQRTHTGKKTS

SEQ ID NO: 826 - protein, 229 aa, synthetic construct
  REGION 1..229: note = source = /note="Description of Artificial Sequence: Syntheticpolypeptide"
LEPGEKPYKCPECGKSFSRRDELNVHQRTHTGEKPYKCPECGKSFSRSDHLTTHQRTHTGEKPYKCPECGKSFSRSDELVRHQRTHTHPRAPIPKPFQYKCPECGKSFSQNSTLTEHQRTHTPNPHRRTDPSHKPFQYKCPECGKSFSRKDNLKNHQRTHTGEKPYKCPECGKSFSRNDALTEHQRTHTHPRAPIPKPFQYKCPECGKSFSQSSNLVRHQRTHTGKKTS

SEQ ID NO: 827 - protein, 204 aa, synthetic construct
  REGION 1..204: note = source = /note="Description of Artificial Sequence: Syntheticpolypeptide"
LEPGEKPYKCPECGKSFSRNDALTEHQRTHTGEKPYKCPECGKSFSTTGNLTVHQRTHTGEKPYKCPECGKSFSRSDNLVRHQRTHTGEKPYKCPECGKSFSQRANLRAHQRTHTGEKPYKCPECGKSFSRKDNLKNHQRTHTGEKPYKCPECGKSFSQRAHLERHQRTHTGEKPYKCPECGKSFSRSDHLTTHQRTHTGKKTS

SEQ ID NO: 828 - protein, 204 aa, synthetic construct
  REGION 1..204: note = source = /note="Description of Artificial Sequence: Syntheticpolypeptide"
LEPGEKPYKCPECGKSFSRKDNLKNHQRTHTGEKPYKCPECGKSFSRNDALTEHQRTHTGEKPYKCPECGKSFSTTGNLTVHQRTHTGEKPYKCPECGKSFSRSDNLVRHQRTHTGEKPYKCPECGKSFSQRANLRAHQRTHTGEKPYKCPECGKSFSRKDNLKNHQRTHTGEKPYKCPECGKSFSQRAHLERHQRTHTGKKTS

SEQ ID NO: 829 - protein, 215 aa, synthetic construct
  REGION 1..215: note = source = /note="Description of Artificial Sequence: Syntheticpolypeptide"
LEPGEKPYKCPECGKSFSQNSTLTEHQRTHTPNPHRRTDPSHKPFQYKCPECGKSFSRKDNLKNHQRTHTGEKPYKCPECGKSFSRNDALTEHQRTHTGEKPYKCPECGKSFSTTGNLTVHQRTHTGEKPYKCPECGKSFSRSDNLVRHQRTHTGEKPYKCPECGKSFSQRANLRAHQRTHTGEKPYKCPECGKSFSRKDNLKNHQRTHTGKKTS

SEQ ID NO: 830 - protein, 222 aa, synthetic construct
  REGION 1..222: note = source = /note="Description of Artificial Sequence: Syntheticpolypeptide"
LEPGEKPYKCPECGKSFSRSDELVRHQRTHTHPRAPIPKPFQYKCPECGKSFSQNSTLTEHQRTHTPNPHRRTDPSHKPFQYKCPECGKSFSRKDNLKNHQRTHTGEKPYKCPECGKSFSRNDALTEHQRTHTGEKPYKCPECGKSFSTTGNLTVHQRTHTGEKPYKCPECGKSFSRSDNLVRHQRTHTGEKPYKCPECGKSFSQRANLRAHQRTHTGKKTS

SEQ ID NO: 831 - protein, 222 aa, synthetic construct
  REGION 1..222: note = source = /note="Description of Artificial Sequence: Syntheticpolypeptide"
LEPGEKPYKCPECGKSFSRSDHLTTHQRTHTGEKPYKCPECGKSFSRSDELVRHQRTHTHPRAPIPKPFQYKCPECGKSFSQNSTLTEHQRTHTPNPHRRTDPSHKPFQYKCPECGKSFSRKDNLKNHQRTHTGEKPYKCPECGKSFSRNDALTEHQRTHTGEKPYKCPECGKSFSTTGNLTVHQRTHTGEKPYKCPECGKSFSRSDNLVRHQRTHTGKKTS

SEQ ID NO: 832 - protein, 222 aa, synthetic construct
  REGION 1..222: note = source = /note="Description of Artificial Sequence: Syntheticpolypeptide"
LEPGEKPYKCPECGKSFSRRDELNVHQRTHTGEKPYKCPECGKSFSRSDHLTTHQRTHTGEKPYKCPECGKSFSRSDELVRHQRTHTHPRAPIPKPFQYKCPECGKSFSQNSTLTEHQRTHTPNPHRRTDPSHKPFQYKCPECGKSFSRKDNLKNHQRTHTGEKPYKCPECGKSFSRNDALTEHQRTHTGEKPYKCPECGKSFSTTGNLTVHQRTHTGKKTS

SEQ ID NO: 833 - protein, 222 aa, synthetic construct
  REGION 1..222: note = source = /note="Description of Artificial Sequence: Syntheticpolypeptide"
LEPGEKPYKCPECGKSFSQRAHLERHQRTHTGEKPYKCPECGKSFSRRDELNVHQRTHTGEKPYKCPECGKSFSRSDHLTTHQRTHTGEKPYKCPECGKSFSRSDELVRHQRTHTHPRAPIPKPFQYKCPECGKSFSQNSTLTEHQRTHTPNPHRRTDPSHKPFQYKCPECGKSFSRKDNLKNHQRTHTGEKPYKCPECGKSFSRNDALTEHQRTHTGKKTS

SEQ ID NO: 834 - protein, 222 aa, synthetic construct
  REGION 1..222: note = source = /note="Description of Artificial Sequence: Syntheticpolypeptide"
LEPGEKPYKCPECGKSFSTTGNLTVHQRTHTGEKPYKCPECGKSFSQRAHLERHQRTHTGEKPYKCPECGKSFSRRDELNVHQRTHTGEKPYKCPECGKSFSRSDHLTTHQRTHTGEKPYKCPECGKSFSRSDELVRHQRTHTHPRAPIPKPFQYKCPECGKSFSQNSTLTEHQRTHTPNPHRRTDPSHKPFQYKCPECGKSFSRKDNLKNHQRTHTGKKTS

SEQ ID NO: 835 - protein, 215 aa, synthetic construct
  REGION 1..215: note = source = /note="Description of Artificial Sequence: Syntheticpolypeptide"
LEPGEKPYKCPECGKSFSRKDNLKNHQRTHTGEKPYKCPECGKSFSRNDALTEHQRTHTPNPHRRTDPSHKPFQYKCPECGKSFSQLAHLRAHQRTHTGEKPYKCPECGKSFSRKDNLKNHQRTHTGEKPYKCPECGKSFSQLAHLRAHQRTHTGEKPYKCPECGKSFSQSSNLVRHQRTHTGEKPYKCPECGKSFSRSDKLVRHQRTHTGKKTS

SEQ ID NO: 836 - protein, 226 aa, synthetic construct
  REGION 1..226: note = source = /note="Description of Artificial Sequence: Syntheticpolypeptide"
LEPGEKPYKCPECGKSFSQNSTLTEHQRTHTPNPHRRTDPSHKPFQYKCPECGKSFSRKDNLKNHQRTHTGEKPYKCPECGKSFSRNDALTEHQRTHTPNPHRRTDPSHKPFQYKCPECGKSFSQLAHLRAHQRTHTGEKPYKCPECGKSFSRKDNLKNHQRTHTGEKPYKCPECGKSFSQLAHLRAHQRTHTGEKPYKCPECGKSFSQSSNLVRHQRTHTGKKTS

SEQ ID NO: 837 - protein, 233 aa, synthetic construct
  REGION 1..233: note = source = /note="Description of Artificial Sequence: Syntheticpolypeptide"
LEPGEKPYKCPECGKSFSRSDELVRHQRTHTHPRAPIPKPFQYKCPECGKSFSQNSTLTEHQRTHTPNPHRRTDPSHKPFQYKCPECGKSFSRKDNLKNHQRTHTGEKPYKCPECGKSFSRNDALTEHQRTHTPNPHRRTDPSHKPFQYKCPECGKSFSQLAHLRAHQRTHTGEKPYKCPECGKSFSRKDNLKNHQRTHTGEKPYKCPECGKSFSQLAHLRAHQRTHTGKKTS

SEQ ID NO: 838 - protein, 233 aa, synthetic construct
  REGION 1..233: note = source = /note="Description of Artificial Sequence: Syntheticpolypeptide"
LEPGEKPYKCPECGKSFSRSDHLTTHQRTHTGEKPYKCPECGKSFSRSDELVRHQRTHTHPRAPIPKPFQYKCPECGKSFSQNSTLTEHQRTHTPNPHRRTDPSHKPFQYKCPECGKSFSRKDNLKNHQRTHTGEKPYKCPECGKSFSRNDALTEHQRTHTPNPHRRTDPSHKPFQYKCPECGKSFSQLAHLRAHQRTHTGEKPYKCPECGKSFSRKDNLKNHQRTHTGKKTS

SEQ ID NO: 839 - protein, 233 aa, synthetic construct
  REGION 1..233: note = source = /note="Description of Artificial Sequence: Syntheticpolypeptide"
LEPGEKPYKCPECGKSFSRRDELNVHQRTHTGEKPYKCPECGKSFSRSDHLTTHQRTHTGEKPYKCPECGKSFSRSDELVRHQRTHTHPRAPIPKPFQYKCPECGKSFSQNSTLTEHQRTHTPNPHRRTDPSHKPFQYKCPECGKSFSRKDNLKNHQRTHTGEKPYKCPECGKSFSRNDALTEHQRTHTPNPHRRTDPSHKPFQYKCPECGKSFSQLAHLRAHQRTHTGKKTS

SEQ ID NO: 840 - protein, 218 aa, synthetic construct
  REGION 1..218: note = source = /note="Description of Artificial Sequence: Syntheticpolypeptide"
LEPGEKPYKCPECGKSFSQRAHLERHQRTHTGEKPYKCPECGKSFSRRDELNVHQRTHTGEKPYKCPECGKSFSRSDHLTTHQRTHTGEKPYKCPECGKSFSRSDELVRHQRTHTHPRAPIPKPFQYKCPECGKSFSQNSTLTEHQRTHTHPRAPIPKPFQYKCPECGKSFSERSHLREHQRTHTGEKPYKCPECGKSFSQAGHLASHQRTHTGKKTS

SEQ ID NO: 841 - protein, 218 aa, synthetic construct
  REGION 1..218: note = source = /note="Description of Artificial Sequence: Syntheticpolypeptide"
LEPGEKPYKCPECGKSFSTTGNLTVHQRTHTGEKPYKCPECGKSFSQRAHLERHQRTHTGEKPYKCPECGKSFSRRDELNVHQRTHTGEKPYKCPECGKSFSRSDHLTTHQRTHTGEKPYKCPECGKSFSRSDELVRHQRTHTHPRAPIPKPFQYKCPECGKSFSQNSTLTEHQRTHTHPRAPIPKPFQYKCPECGKSFSERSHLREHQRTHTGKKTS

SEQ ID NO: 842 - protein, 211 aa, synthetic construct
  REGION 1..211: note = source = /note="Description of Artificial Sequence: Syntheticpolypeptide"
LEPGEKPYKCPECGKSFSQRAHLERHQRTHTGEKPYKCPECGKSFSRRDELNVHQRTHTGEKPYKCPECGKSFSRSDHLTTHQRTHTGEKPYKCPECGKSFSRSDELVRHQRTHTHPRAPIPKPFQYKCPECGKSFSQNSTLTEHQRTHTGEKPYKCPECGKSFSTSGELVRHQRTHTGEKPYKCPECGKSFSQSSNLVRHQRTHTGKKTS

SEQ ID NO: 843 - protein, 211 aa, synthetic construct
  REGION 1..211: note = source = /note="Description of Artificial Sequence: Syntheticpolypeptide"
LEPGEKPYKCPECGKSFSTTGNLTVHQRTHTGEKPYKCPECGKSFSQRAHLERHQRTHTGEKPYKCPECGKSFSRRDELNVHQRTHTGEKPYKCPECGKSFSRSDHLTTHQRTHTGEKPYKCPECGKSFSRSDELVRHQRTHTHPRAPIPKPFQYKCPECGKSFSQNSTLTEHQRTHTGEKPYKCPECGKSFSTSGELVRHQRTHTGKKTS

SEQ ID NO: 844 - protein, 211 aa, synthetic construct
  REGION 1..211: note = source = /note="Description of Artificial Sequence: Syntheticpolypeptide"
LEPGEKPYKCPECGKSFSDCRDLARHQRTHTGEKPYKCPECGKSFSTTGNLTVHQRTHTGEKPYKCPECGKSFSQRAHLERHQRTHTGEKPYKCPECGKSFSRRDELNVHQRTHTGEKPYKCPECGKSFSRSDHLTTHQRTHTGEKPYKCPECGKSFSRSDELVRHQRTHTHPRAPIPKPFQYKCPECGKSFSQNSTLTEHQRTHTGKKTS

SEQ ID NO: 845 - protein, 218 aa, synthetic construct
  REGION 1..218: note = source = /note="Description of Artificial Sequence: Syntheticpolypeptide"
LEPGEKPYKCPECGKSFSRSDHLTNHQRTHTGEKPYKCPECGKSFSDCRDLARHQRTHTHPRAPIPKPFQYKCPECGKSFSQRANLRAHQRTHTGEKPYKCPECGKSFSRSDKLVRHQRTHTGEKPYKCPECGKSFSTSGNLVRHQRTHTGEKPYKCPECGKSFSRSDELVRHQRTHTHPRAPIPKPFQYKCPECGKSFSRRDELNVHQRTHTGKKTS

SEQ ID NO: 846 - protein, 222 aa, synthetic construct
  REGION 1..222: note = source = /note="Description of Artificial Sequence: Syntheticpolypeptide"
LEPGEKPYKCPECGKSFSTTGALTEHQRTHTGEKPYKCPECGKSFSRSDNLVRHQRTHTGEKPYKCPECGKSFSQKSSLIAHQRTHTGEKPYKCPECGKSFSSKKALTEHQRTHTGEKPYKCPECGKSFSTSHSLTEHQRTHTPNPHRRTDPSHKPFQYKCPECGKSFSSKKHLAEHQRTHTHPRAPIPKPFQYKCPECGKSFSHKNALQNHQRTHTGKKTS

SEQ ID NO: 847 - protein, 215 aa, synthetic construct
  REGION 1..215: note = source = /note="Description of Artificial Sequence: Syntheticpolypeptide"
LEPGEKPYKCPECGKSFSRADNLTEHQRTHTGEKPYKCPECGKSFSTTGALTEHQRTHTGEKPYKCPECGKSFSRSDNLVRHQRTHTGEKPYKCPECGKSFSQKSSLIAHQRTHTGEKPYKCPECGKSFSSKKALTEHQRTHTGEKPYKCPECGKSFSTSHSLTEHQRTHTPNPHRRTDPSHKPFQYKCPECGKSFSSKKHLAEHQRTHTGKKTS

SEQ ID NO: 848 - protein, 211 aa, synthetic construct
  REGION 1..211: note = source = /note="Description of Artificial Sequence: Syntheticpolypeptide"
LEPGEKPYKCPECGKSFSRADNLTEHQRTHTGEKPYKCPECGKSFSTTGALTEHQRTHTGEKPYKCPECGKSFSRSDNLVRHQRTHTGEKPYKCPECGKSFSQKSSLIAHQRTHTGEKPYKCPECGKSFSSKKALTEHQRTHTGEKPYKCPECGKSFSTSHSLTEHQRTHTHPRAPIPKPFQYKCPECGKSFSTSHSLTEHQRTHTGKKTS

SEQ ID NO: 849 - protein, 204 aa, synthetic construct
  REGION 1..204: note = source = /note="Description of Artificial Sequence: Syntheticpolypeptide"
LEPGEKPYKCPECGKSFSRADNLTEHQRTHTGEKPYKCPECGKSFSTTGALTEHQRTHTGEKPYKCPECGKSFSRSDNLVRHQRTHTGEKPYKCPECGKSFSQKSSLIAHQRTHTGEKPYKCPECGKSFSSKKALTEHQRTHTGEKPYKCPECGKSFSTSHSLTEHQRTHTGEKPYKCPECGKSFSTSGNLTEHQRTHTGKKTS

SEQ ID NO: 850 - protein, 204 aa, synthetic construct
  REGION 1..204: note = source = /note="Description of Artificial Sequence: Syntheticpolypeptide"
LEPGEKPYKCPECGKSFSHKNALQNHQRTHTGEKPYKCPECGKSFSRADNLTEHQRTHTGEKPYKCPECGKSFSTTGALTEHQRTHTGEKPYKCPECGKSFSRSDNLVRHQRTHTGEKPYKCPECGKSFSQKSSLIAHQRTHTGEKPYKCPECGKSFSSKKALTEHQRTHTGEKPYKCPECGKSFSTSHSLTEHQRTHTGKKTS

SEQ ID NO: 851 - protein, 215 aa, synthetic construct
  REGION 1..215: note = source = /note="Description of Artificial Sequence: Syntheticpolypeptide"
LEPGEKPYKCPECGKSFSTTGALTEHQRTHTPNPHRRTDPSHKPFQYKCPECGKSFSHKNALQNHQRTHTGEKPYKCPECGKSFSRADNLTEHQRTHTGEKPYKCPECGKSFSTTGALTEHQRTHTGEKPYKCPECGKSFSRSDNLVRHQRTHTGEKPYKCPECGKSFSQKSSLIAHQRTHTGEKPYKCPECGKSFSSKKALTEHQRTHTGKKTS

SEQ ID NO: 852 - protein, 222 aa, synthetic construct
  REGION 1..222: note = source = /note="Description of Artificial Sequence: Syntheticpolypeptide"
LEPGEKPYKCPECGKSFSTTGALTEHQRTHTHPRAPIPKPFQYKCPECGKSFSTTGALTEHQRTHTPNPHRRTDPSHKPFQYKCPECGKSFSHKNALQNHQRTHTGEKPYKCPECGKSFSRADNLTEHQRTHTGEKPYKCPECGKSFSTTGALTEHQRTHTGEKPYKCPECGKSFSRSDNLVRHQRTHTGEKPYKCPECGKSFSQKSSLIAHQRTHTGKKTS

SEQ ID NO: 853 - protein, 229 aa, synthetic construct
  REGION 1..229: note = source = /note="Description of Artificial Sequence: Syntheticpolypeptide"
LEPGEKPYKCPECGKSFSSKKHLAEHQRTHTHPRAPIPKPFQYKCPECGKSFSTTGALTEHQRTHTHPRAPIPKPFQYKCPECGKSFSTTGALTEHQRTHTPNPHRRTDPSHKPFQYKCPECGKSFSHKNALQNHQRTHTGEKPYKCPECGKSFSRADNLTEHQRTHTGEKPYKCPECGKSFSTTGALTEHQRTHTGEKPYKCPECGKSFSRSDNLVRHQRTHTGKKTS

SEQ ID NO: 854 - protein, 229 aa, synthetic construct
  REGION 1..229: note = source = /note="Description of Artificial Sequence: Syntheticpolypeptide"
LEPGEKPYKCPECGKSFSRADNLTEHQRTHTGEKPYKCPECGKSFSSKKHLAEHQRTHTHPRAPIPKPFQYKCPECGKSFSTTGALTEHQRTHTHPRAPIPKPFQYKCPECGKSFSTTGALTEHQRTHTPNPHRRTDPSHKPFQYKCPECGKSFSHKNALQNHQRTHTGEKPYKCPECGKSFSRADNLTEHQRTHTGEKPYKCPECGKSFSTTGALTEHQRTHTGKKTS

SEQ ID NO: 855 - protein, 229 aa, synthetic construct
  REGION 1..229: note = source = /note="Description of Artificial Sequence: Syntheticpolypeptide"
LEPGEKPYKCPECGKSFSQLAHLRAHQRTHTGEKPYKCPECGKSFSRADNLTEHQRTHTGEKPYKCPECGKSFSSKKHLAEHQRTHTHPRAPIPKPFQYKCPECGKSFSTTGALTEHQRTHTHPRAPIPKPFQYKCPECGKSFSTTGALTEHQRTHTPNPHRRTDPSHKPFQYKCPECGKSFSHKNALQNHQRTHTGEKPYKCPECGKSFSRADNLTEHQRTHTGKKTS

SEQ ID NO: 856 - protein, 229 aa, synthetic construct
  REGION 1..229: note = source = /note="Description of Artificial Sequence: Syntheticpolypeptide"
LEPGEKPYKCPECGKSFSSKKHLAEHQRTHTGEKPYKCPECGKSFSQLAHLRAHQRTHTGEKPYKCPECGKSFSRADNLTEHQRTHTGEKPYKCPECGKSFSSKKHLAEHQRTHTHPRAPIPKPFQYKCPECGKSFSTTGALTEHQRTHTHPRAPIPKPFQYKCPECGKSFSTTGALTEHQRTHTPNPHRRTDPSHKPFQYKCPECGKSFSHKNALQNHQRTHTGKKTS

SEQ ID NO: 857 - protein, 226 aa, synthetic construct
  REGION 1..226: note = source = /note="Description of Artificial Sequence: Syntheticpolypeptide"
LEPGEKPYKCPECGKSFSTTGALTEHQRTHTPNPHRRTDPSHKPFQYKCPECGKSFSHKNALQNHQRTHTGEKPYKCPECGKSFSRADNLTEHQRTHTPNPHRRTDPSHKPFQYKCPECGKSFSERSHLREHQRTHTGEKPYKCPECGKSFSREDNLHTHQRTHTGEKPYKCPECGKSFSSPADLTRHQRTHTGEKPYKCPECGKSFSSKKALTEHQRTHTGKKTS

SEQ ID NO: 858 - protein, 233 aa, synthetic construct
  REGION 1..233: note = source = /note="Description of Artificial Sequence: Syntheticpolypeptide"
LEPGEKPYKCPECGKSFSTTGALTEHQRTHTHPRAPIPKPFQYKCPECGKSFSTTGALTEHQRTHTPNPHRRTDPSHKPFQYKCPECGKSFSHKNALQNHQRTHTGEKPYKCPECGKSFSRADNLTEHQRTHTPNPHRRTDPSHKPFQYKCPECGKSFSERSHLREHQRTHTGEKPYKCPECGKSFSREDNLHTHQRTHTGEKPYKCPECGKSFSSPADLTRHQRTHTGKKTS

SEQ ID NO: 859 - protein, 240 aa, synthetic construct
  REGION 1..240: note = source = /note="Description of Artificial Sequence: Syntheticpolypeptide"
LEPGEKPYKCPECGKSFSSKKHLAEHQRTHTHPRAPIPKPFQYKCPECGKSFSTTGALTEHQRTHTHPRAPIPKPFQYKCPECGKSFSTTGALTEHQRTHTPNPHRRTDPSHKPFQYKCPECGKSFSHKNALQNHQRTHTGEKPYKCPECGKSFSRADNLTEHQRTHTPNPHRRTDPSHKPFQYKCPECGKSFSERSHLREHQRTHTGEKPYKCPECGKSFSREDNLHTHQRTHTGKKTS

SEQ ID NO: 860 - protein, 240 aa, synthetic construct
  REGION 1..240: note = source = /note="Description of Artificial Sequence: Syntheticpolypeptide"
LEPGEKPYKCPECGKSFSRADNLTEHQRTHTGEKPYKCPECGKSFSSKKHLAEHQRTHTHPRAPIPKPFQYKCPECGKSFSTTGALTEHQRTHTHPRAPIPKPFQYKCPECGKSFSTTGALTEHQRTHTPNPHRRTDPSHKPFQYKCPECGKSFSHKNALQNHQRTHTGEKPYKCPECGKSFSRADNLTEHQRTHTPNPHRRTDPSHKPFQYKCPECGKSFSERSHLREHQRTHTGKKTS

SEQ ID NO: 861 - protein, 222 aa, synthetic construct
  REGION 1..222: note = source = /note="Description of Artificial Sequence: Syntheticpolypeptide"
LEPGEKPYKCPECGKSFSTTGALTEHQRTHTPNPHRRTDPSHKPFQYKCPECGKSFSHKNALQNHQRTHTGEKPYKCPECGKSFSRADNLTEHQRTHTHPRAPIPKPFQYKCPECGKSFSTSGELVRHQRTHTGEKPYKCPECGKSFSQLAHLRAHQRTHTGEKPYKCPECGKSFSTSGNLTEHQRTHTGEKPYKCPECGKSFSSPADLTRHQRTHTGKKTS

SEQ ID NO: 862 - protein, 229 aa, synthetic construct
  REGION 1..229: note = source = /note="Description of Artificial Sequence: Syntheticpolypeptide"
LEPGEKPYKCPECGKSFSTTGALTEHQRTHTHPRAPIPKPFQYKCPECGKSFSTTGALTEHQRTHTPNPHRRTDPSHKPFQYKCPECGKSFSHKNALQNHQRTHTGEKPYKCPECGKSFSRADNLTEHQRTHTHPRAPIPKPFQYKCPECGKSFSTSGELVRHQRTHTGEKPYKCPECGKSFSQLAHLRAHQRTHTGEKPYKCPECGKSFSTSGNLTEHQRTHTGKKTS

SEQ ID NO: 863 - protein, 236 aa, synthetic construct
  REGION 1..236: note = source = /note="Description of Artificial Sequence: Syntheticpolypeptide"
LEPGEKPYKCPECGKSFSSKKHLAEHQRTHTHPRAPIPKPFQYKCPECGKSFSTTGALTEHQRTHTHPRAPIPKPFQYKCPECGKSFSTTGALTEHQRTHTPNPHRRTDPSHKPFQYKCPECGKSFSHKNALQNHQRTHTGEKPYKCPECGKSFSRADNLTEHQRTHTHPRAPIPKPFQYKCPECGKSFSTSGELVRHQRTHTGEKPYKCPECGKSFSQLAHLRAHQRTHTGKKTS

SEQ ID NO: 864 - protein, 236 aa, synthetic construct
  REGION 1..236: note = source = /note="Description of Artificial Sequence: Syntheticpolypeptide"
LEPGEKPYKCPECGKSFSRADNLTEHQRTHTGEKPYKCPECGKSFSSKKHLAEHQRTHTHPRAPIPKPFQYKCPECGKSFSTTGALTEHQRTHTHPRAPIPKPFQYKCPECGKSFSTTGALTEHQRTHTPNPHRRTDPSHKPFQYKCPECGKSFSHKNALQNHQRTHTGEKPYKCPECGKSFSRADNLTEHQRTHTHPRAPIPKPFQYKCPECGKSFSTSGELVRHQRTHTGKKTS

SEQ ID NO: 865 - protein, 225 aa, synthetic construct
  REGION 1..225: note = source = /note="Description of Artificial Sequence: Syntheticpolypeptide"
LEPGEKPYKCPECGKSFSQSGNLTEHQRTHTHPRAPIPKPFQYKCPECGKSFSSKKHLAEHQRTHTGEKPYKCPECGKSFSQLAHLRAHQRTHTGEKPYKCPECGKSFSRADNLTEHQRTHTGEKPYKCPECGKSFSSKKHLAEHQRTHTHPRAPIPKPFQYKCPECGKSFSTTGALTEHQRTHTHPRAPIPKPFQYKCPECGKSFSTTGALTEHQRTHTGKKTS

SEQ ID NO: 866 - protein, 218 aa, synthetic construct
  REGION 1..218: note = source = /note="Description of Artificial Sequence: Syntheticpolypeptide"
LEPGEKPYKCPECGKSFSQKSSLIAHQRTHTGEKPYKCPECGKSFSQSGNLTEHQRTHTHPRAPIPKPFQYKCPECGKSFSSKKHLAEHQRTHTGEKPYKCPECGKSFSQLAHLRAHQRTHTGEKPYKCPECGKSFSRADNLTEHQRTHTGEKPYKCPECGKSFSSKKHLAEHQRTHTHPRAPIPKPFQYKCPECGKSFSTTGALTEHQRTHTGKKTS

SEQ ID NO: 867 - protein, 218 aa, synthetic construct
  REGION 1..218: note = source = /note="Description of Artificial Sequence: Syntheticpolypeptide"
LEPGEKPYKCPECGKSFSTKNSLTEHQRTHTHPRAPIPKPFQYKCPECGKSFSQKSSLIAHQRTHTGEKPYKCPECGKSFSQSGNLTEHQRTHTHPRAPIPKPFQYKCPECGKSFSSKKHLAEHQRTHTGEKPYKCPECGKSFSQLAHLRAHQRTHTGEKPYKCPECGKSFSRADNLTEHQRTHTGEKPYKCPECGKSFSSKKHLAEHQRTHTGKKTS

SEQ ID NO: 868 - protein, 229 aa, synthetic construct
  REGION 1..229: note = source = /note="Description of Artificial Sequence: Syntheticpolypeptide"
LEPGEKPYKCPECGKSFSSKKALTEHQRTHTPNPHRRTDPSHKPFQYKCPECGKSFSTKNSLTEHQRTHTHPRAPIPKPFQYKCPECGKSFSQKSSLIAHQRTHTGEKPYKCPECGKSFSQSGNLTEHQRTHTHPRAPIPKPFQYKCPECGKSFSSKKHLAEHQRTHTGEKPYKCPECGKSFSQLAHLRAHQRTHTGEKPYKCPECGKSFSRADNLTEHQRTHTGKKTS

SEQ ID NO: 869 - protein, 236 aa, synthetic construct
  REGION 1..236: note = source = /note="Description of Artificial Sequence: Syntheticpolypeptide"
LEPGEKPYKCPECGKSFSSKKALTEHQRTHTHPRAPIPKPFQYKCPECGKSFSSKKALTEHQRTHTPNPHRRTDPSHKPFQYKCPECGKSFSTKNSLTEHQRTHTHPRAPIPKPFQYKCPECGKSFSQKSSLIAHQRTHTGEKPYKCPECGKSFSQSGNLTEHQRTHTHPRAPIPKPFQYKCPECGKSFSSKKHLAEHQRTHTGEKPYKCPECGKSFSQLAHLRAHQRTHTGKKTS

SEQ ID NO: 870 - protein, 236 aa, synthetic construct
  REGION 1..236: note = source = /note="Description of Artificial Sequence: Syntheticpolypeptide"
LEPGEKPYKCPECGKSFSQLAHLRAHQRTHTGEKPYKCPECGKSFSSKKALTEHQRTHTHPRAPIPKPFQYKCPECGKSFSSKKALTEHQRTHTPNPHRRTDPSHKPFQYKCPECGKSFSTKNSLTEHQRTHTHPRAPIPKPFQYKCPECGKSFSQKSSLIAHQRTHTGEKPYKCPECGKSFSQSGNLTEHQRTHTHPRAPIPKPFQYKCPECGKSFSSKKHLAEHQRTHTGKKTS

SEQ ID NO: 871 - protein, 211 aa, synthetic construct
  REGION 1..211: note = source = /note="Description of Artificial Sequence: Syntheticpolypeptide"
LEPGEKPYKCPECGKSFSTKNSLTEHQRTHTHPRAPIPKPFQYKCPECGKSFSQKSSLIAHQRTHTGEKPYKCPECGKSFSQSGNLTEHQRTHTGEKPYKCPECGKSFSTKNSLTEHQRTHTGEKPYKCPECGKSFSDPGNLVRHQRTHTGEKPYKCPECGKSFSQLAHLRAHQRTHTGEKPYKCPECGKSFSSKKHLAEHQRTHTGKKTS

SEQ ID NO: 872 - protein, 222 aa, synthetic construct
  REGION 1..222: note = source = /note="Description of Artificial Sequence: Syntheticpolypeptide"
LEPGEKPYKCPECGKSFSSKKALTEHQRTHTPNPHRRTDPSHKPFQYKCPECGKSFSTKNSLTEHQRTHTHPRAPIPKPFQYKCPECGKSFSQKSSLIAHQRTHTGEKPYKCPECGKSFSQSGNLTEHQRTHTGEKPYKCPECGKSFSTKNSLTEHQRTHTGEKPYKCPECGKSFSDPGNLVRHQRTHTGEKPYKCPECGKSFSQLAHLRAHQRTHTGKKTS

SEQ ID NO: 873 - protein, 229 aa, synthetic construct
  REGION 1..229: note = source = /note="Description of Artificial Sequence: Syntheticpolypeptide"
LEPGEKPYKCPECGKSFSSKKALTEHQRTHTHPRAPIPKPFQYKCPECGKSFSSKKALTEHQRTHTPNPHRRTDPSHKPFQYKCPECGKSFSTKNSLTEHQRTHTHPRAPIPKPFQYKCPECGKSFSQKSSLIAHQRTHTGEKPYKCPECGKSFSQSGNLTEHQRTHTGEKPYKCPECGKSFSTKNSLTEHQRTHTGEKPYKCPECGKSFSDPGNLVRHQRTHTGKKTS

SEQ ID NO: 874 - protein, 229 aa, synthetic construct
  REGION 1..229: note = source = /note="Description of Artificial Sequence: Syntheticpolypeptide"
LEPGEKPYKCPECGKSFSQLAHLRAHQRTHTGEKPYKCPECGKSFSSKKALTEHQRTHTHPRAPIPKPFQYKCPECGKSFSSKKALTEHQRTHTPNPHRRTDPSHKPFQYKCPECGKSFSTKNSLTEHQRTHTHPRAPIPKPFQYKCPECGKSFSQKSSLIAHQRTHTGEKPYKCPECGKSFSQSGNLTEHQRTHTGEKPYKCPECGKSFSTKNSLTEHQRTHTGKKTS

SEQ ID NO: 875 - protein, 229 aa, synthetic construct
  REGION 1..229: note = source = /note="Description of Artificial Sequence: Syntheticpolypeptide"
LEPGEKPYKCPECGKSFSRRDELNVHQRTHTGEKPYKCPECGKSFSQLAHLRAHQRTHTGEKPYKCPECGKSFSSKKALTEHQRTHTHPRAPIPKPFQYKCPECGKSFSSKKALTEHQRTHTPNPHRRTDPSHKPFQYKCPECGKSFSTKNSLTEHQRTHTHPRAPIPKPFQYKCPECGKSFSQKSSLIAHQRTHTGEKPYKCPECGKSFSQSGNLTEHQRTHTGKKTS

SEQ ID NO: 876 - protein, 229 aa, synthetic construct
  REGION 1..229: note = source = /note="Description of Artificial Sequence: Syntheticpolypeptide"
LEPGEKPYKCPECGKSFSRSDELVRHQRTHTGEKPYKCPECGKSFSRRDELNVHQRTHTGEKPYKCPECGKSFSQLAHLRAHQRTHTGEKPYKCPECGKSFSSKKALTEHQRTHTHPRAPIPKPFQYKCPECGKSFSSKKALTEHQRTHTPNPHRRTDPSHKPFQYKCPECGKSFSTKNSLTEHQRTHTHPRAPIPKPFQYKCPECGKSFSQKSSLIAHQRTHTGKKTS

SEQ ID NO: 877 - protein, 233 aa, synthetic construct
  REGION 1..233: note = source = /note="Description of Artificial Sequence: Syntheticpolypeptide"
LEPGEKPYKCPECGKSFSSKKALTEHQRTHTPNPHRRTDPSHKPFQYKCPECGKSFSTKNSLTEHQRTHTHPRAPIPKPFQYKCPECGKSFSQKSSLIAHQRTHTGEKPYKCPECGKSFSQSGNLTEHQRTHTPNPHRRTDPSHKPFQYKCPECGKSFSDKKDLTRHQRTHTGEKPYKCPECGKSFSRSDNLVRHQRTHTGEKPYKCPECGKSFSTSHSLTEHQRTHTGKKTS

SEQ ID NO: 878 - protein, 240 aa, synthetic construct
  REGION 1..240: note = source = /note="Description of Artificial Sequence: Syntheticpolypeptide"
LEPGEKPYKCPECGKSFSSKKALTEHQRTHTHPRAPIPKPFQYKCPECGKSFSSKKALTEHQRTHTPNPHRRTDPSHKPFQYKCPECGKSFSTKNSLTEHQRTHTHPRAPIPKPFQYKCPECGKSFSQKSSLIAHQRTHTGEKPYKCPECGKSFSQSGNLTEHQRTHTPNPHRRTDPSHKPFQYKCPECGKSFSDKKDLTRHQRTHTGEKPYKCPECGKSFSRSDNLVRHQRTHTGKKTS

SEQ ID NO: 879 - protein, 240 aa, synthetic construct
  REGION 1..240: note = source = /note="Description of Artificial Sequence: Syntheticpolypeptide"
LEPGEKPYKCPECGKSFSQLAHLRAHQRTHTGEKPYKCPECGKSFSSKKALTEHQRTHTHPRAPIPKPFQYKCPECGKSFSSKKALTEHQRTHTPNPHRRTDPSHKPFQYKCPECGKSFSTKNSLTEHQRTHTHPRAPIPKPFQYKCPECGKSFSQKSSLIAHQRTHTGEKPYKCPECGKSFSQSGNLTEHQRTHTPNPHRRTDPSHKPFQYKCPECGKSFSDKKDLTRHQRTHTGKKTS

SEQ ID NO: 880 - protein, 233 aa, synthetic construct
  REGION 1..233: note = source = /note="Description of Artificial Sequence: Syntheticpolypeptide"
LEPGEKPYKCPECGKSFSRSDELVRHQRTHTGEKPYKCPECGKSFSRRDELNVHQRTHTGEKPYKCPECGKSFSQLAHLRAHQRTHTGEKPYKCPECGKSFSSKKALTEHQRTHTHPRAPIPKPFQYKCPECGKSFSSKKALTEHQRTHTPNPHRRTDPSHKPFQYKCPECGKSFSTKNSLTEHQRTHTPNPHRRTDPSHKPFQYKCPECGKSFSTTGNLTVHQRTHTGKKTS

SEQ ID NO: 881 - protein, 218 aa, synthetic construct
  REGION 1..218: note = source = /note="Description of Artificial Sequence: Syntheticpolypeptide"
LEPGEKPYKCPECGKSFSDCRDLARHQRTHTGEKPYKCPECGKSFSTKNSLTEHQRTHTGEKPYKCPECGKSFSRNDALTEHQRTHTHPRAPIPKPFQYKCPECGKSFSDPGALVRHQRTHTHPRAPIPKPFQYKCPECGKSFSRSDHLTTHQRTHTGEKPYKCPECGKSFSQSSNLVRHQRTHTGEKPYKCPECGKSFSSPADLTRHQRTHTGKKTS

SEQ ID NO: 882 - protein, 225 aa, synthetic construct
  REGION 1..225: note = source = /note="Description of Artificial Sequence: Syntheticpolypeptide"
LEPGEKPYKCPECGKSFSRSDKLTEHQRTHTHPRAPIPKPFQYKCPECGKSFSDCRDLARHQRTHTGEKPYKCPECGKSFSTKNSLTEHQRTHTGEKPYKCPECGKSFSRNDALTEHQRTHTHPRAPIPKPFQYKCPECGKSFSDPGALVRHQRTHTHPRAPIPKPFQYKCPECGKSFSRSDHLTTHQRTHTGEKPYKCPECGKSFSQSSNLVRHQRTHTGKKTS

SEQ ID NO: 883 - protein, 225 aa, synthetic construct
  REGION 1..225: note = source = /note="Description of Artificial Sequence: Syntheticpolypeptide"
LEPGEKPYKCPECGKSFSTSGSLVRHQRTHTGEKPYKCPECGKSFSRSDKLTEHQRTHTHPRAPIPKPFQYKCPECGKSFSDCRDLARHQRTHTGEKPYKCPECGKSFSTKNSLTEHQRTHTGEKPYKCPECGKSFSRNDALTEHQRTHTHPRAPIPKPFQYKCPECGKSFSDPGALVRHQRTHTHPRAPIPKPFQYKCPECGKSFSRSDHLTTHQRTHTGKKTS

SEQ ID NO: 884 - protein, 211 aa, synthetic construct
  REGION 1..211: note = source = /note="Description of Artificial Sequence: Syntheticpolypeptide"
LEPGEKPYKCPECGKSFSDCRDLARHQRTHTGEKPYKCPECGKSFSTKNSLTEHQRTHTGEKPYKCPECGKSFSRNDALTEHQRTHTHPRAPIPKPFQYKCPECGKSFSDPGALVRHQRTHTGEKPYKCPECGKSFSTSGHLVRHQRTHTGEKPYKCPECGKSFSTTGNLTVHQRTHTGEKPYKCPECGKSFSRADNLTEHQRTHTGKKTS

SEQ ID NO: 885 - protein, 218 aa, synthetic construct
  REGION 1..218: note = source = /note="Description of Artificial Sequence: Syntheticpolypeptide"
LEPGEKPYKCPECGKSFSRSDKLTEHQRTHTHPRAPIPKPFQYKCPECGKSFSDCRDLARHQRTHTGEKPYKCPECGKSFSTKNSLTEHQRTHTGEKPYKCPECGKSFSRNDALTEHQRTHTHPRAPIPKPFQYKCPECGKSFSDPGALVRHQRTHTGEKPYKCPECGKSFSTSGHLVRHQRTHTGEKPYKCPECGKSFSTTGNLTVHQRTHTGKKTS

SEQ ID NO: 886 - protein, 218 aa, synthetic construct
  REGION 1..218: note = source = /note="Description of Artificial Sequence: Syntheticpolypeptide"
LEPGEKPYKCPECGKSFSTSGSLVRHQRTHTGEKPYKCPECGKSFSRSDKLTEHQRTHTHPRAPIPKPFQYKCPECGKSFSDCRDLARHQRTHTGEKPYKCPECGKSFSTKNSLTEHQRTHTGEKPYKCPECGKSFSRNDALTEHQRTHTHPRAPIPKPFQYKCPECGKSFSDPGALVRHQRTHTGEKPYKCPECGKSFSTSGHLVRHQRTHTGKKTS

SEQ ID NO: 887 - protein, 218 aa, synthetic construct
  REGION 1..218: note = source = /note="Description of Artificial Sequence: Syntheticpolypeptide"
LEPGEKPYKCPECGKSFSDPGHLVRHQRTHTGEKPYKCPECGKSFSTSGSLVRHQRTHTGEKPYKCPECGKSFSRSDKLTEHQRTHTHPRAPIPKPFQYKCPECGKSFSDCRDLARHQRTHTGEKPYKCPECGKSFSTKNSLTEHQRTHTGEKPYKCPECGKSFSRNDALTEHQRTHTHPRAPIPKPFQYKCPECGKSFSDPGALVRHQRTHTGKKTS

SEQ ID NO: 888 - protein, 211 aa, synthetic construct
  REGION 1..211: note = source = /note="Description of Artificial Sequence: Syntheticpolypeptide"
LEPGEKPYKCPECGKSFSTKNSLTEHQRTHTGEKPYKCPECGKSFSDPGHLVRHQRTHTGEKPYKCPECGKSFSTSGSLVRHQRTHTGEKPYKCPECGKSFSRSDKLTEHQRTHTHPRAPIPKPFQYKCPECGKSFSDCRDLARHQRTHTGEKPYKCPECGKSFSTKNSLTEHQRTHTGEKPYKCPECGKSFSRNDALTEHQRTHTGKKTS

SEQ ID NO: 889 - protein, 211 aa, synthetic construct
  REGION 1..211: note = source = /note="Description of Artificial Sequence: Syntheticpolypeptide"
LEPGEKPYKCPECGKSFSRSDELVRHQRTHTGEKPYKCPECGKSFSTKNSLTEHQRTHTGEKPYKCPECGKSFSDPGHLVRHQRTHTGEKPYKCPECGKSFSTSGSLVRHQRTHTGEKPYKCPECGKSFSRSDKLTEHQRTHTHPRAPIPKPFQYKCPECGKSFSDCRDLARHQRTHTGEKPYKCPECGKSFSTKNSLTEHQRTHTGKKTS

SEQ ID NO: 890 - protein, 211 aa, synthetic construct
  REGION 1..211: note = source = /note="Description of Artificial Sequence: Syntheticpolypeptide"
LEPGEKPYKCPECGKSFSHKNALQNHQRTHTGEKPYKCPECGKSFSRSDELVRHQRTHTGEKPYKCPECGKSFSTKNSLTEHQRTHTGEKPYKCPECGKSFSDPGHLVRHQRTHTGEKPYKCPECGKSFSTSGSLVRHQRTHTGEKPYKCPECGKSFSRSDKLTEHQRTHTHPRAPIPKPFQYKCPECGKSFSDCRDLARHQRTHTGKKTS

SEQ ID NO: 891 - protein, 218 aa, synthetic construct
  REGION 1..218: note = source = /note="Description of Artificial Sequence: Syntheticpolypeptide"
LEPGEKPYKCPECGKSFSRSDELVRHQRTHTGEKPYKCPECGKSFSTKNSLTEHQRTHTGEKPYKCPECGKSFSDPGHLVRHQRTHTGEKPYKCPECGKSFSTSGSLVRHQRTHTGEKPYKCPECGKSFSRSDKLTEHQRTHTHPRAPIPKPFQYKCPECGKSFSDCRDLARHQRTHTHPRAPIPKPFQYKCPECGKSFSDCRDLARHQRTHTGKKTS

SEQ ID NO: 892 - protein, 204 aa, synthetic construct
  REGION 1..204: note = source = /note="Description of Artificial Sequence: Syntheticpolypeptide"
LEPGEKPYKCPECGKSFSRSDELVRHQRTHTGEKPYKCPECGKSFSTKNSLTEHQRTHTGEKPYKCPECGKSFSDPGHLVRHQRTHTGEKPYKCPECGKSFSTSGSLVRHQRTHTGEKPYKCPECGKSFSRSDKLTEHQRTHTGEKPYKCPECGKSFSTKNSLTEHQRTHTGEKPYKCPECGKSFSRNDALTEHQRTHTGKKTS

SEQ ID NO: 893 - protein, 204 aa, synthetic construct
  REGION 1..204: note = source = /note="Description of Artificial Sequence: Syntheticpolypeptide"
LEPGEKPYKCPECGKSFSHKNALQNHQRTHTGEKPYKCPECGKSFSRSDELVRHQRTHTGEKPYKCPECGKSFSTKNSLTEHQRTHTGEKPYKCPECGKSFSDPGHLVRHQRTHTGEKPYKCPECGKSFSTSGSLVRHQRTHTGEKPYKCPECGKSFSRSDKLTEHQRTHTGEKPYKCPECGKSFSTKNSLTEHQRTHTGKKTS

SEQ ID NO: 894 - protein, 211 aa, synthetic construct
  REGION 1..211: note = source = /note="Description of Artificial Sequence: Syntheticpolypeptide"
LEPGEKPYKCPECGKSFSHKNALQNHQRTHTHPRAPIPKPFQYKCPECGKSFSHKNALQNHQRTHTGEKPYKCPECGKSFSRSDELVRHQRTHTGEKPYKCPECGKSFSTKNSLTEHQRTHTGEKPYKCPECGKSFSDPGHLVRHQRTHTGEKPYKCPECGKSFSTSGSLVRHQRTHTGEKPYKCPECGKSFSRSDKLTEHQRTHTGKKTS

SEQ ID NO: 895 - protein, 218 aa, synthetic construct
  REGION 1..218: note = source = /note="Description of Artificial Sequence: Syntheticpolypeptide"
LEPGEKPYKCPECGKSFSREDNLHTHQRTHTHPRAPIPKPFQYKCPECGKSFSHKNALQNHQRTHTHPRAPIPKPFQYKCPECGKSFSHKNALQNHQRTHTGEKPYKCPECGKSFSRSDELVRHQRTHTGEKPYKCPECGKSFSTKNSLTEHQRTHTGEKPYKCPECGKSFSDPGHLVRHQRTHTGEKPYKCPECGKSFSTSGSLVRHQRTHTGKKTS

SEQ ID NO: 896 - protein, 229 aa, synthetic construct
  REGION 1..229: note = source = /note="Description of Artificial Sequence: Syntheticpolypeptide"
LEPGEKPYKCPECGKSFSTTGALTEHQRTHTPNPHRRTDPSHKPFQYKCPECGKSFSREDNLHTHQRTHTHPRAPIPKPFQYKCPECGKSFSHKNALQNHQRTHTHPRAPIPKPFQYKCPECGKSFSHKNALQNHQRTHTGEKPYKCPECGKSFSRSDELVRHQRTHTGEKPYKCPECGKSFSTKNSLTEHQRTHTGEKPYKCPECGKSFSDPGHLVRHQRTHTGKKTS

SEQ ID NO: 897 - protein, 240 aa, synthetic construct
  REGION 1..240: note = source = /note="Description of Artificial Sequence: Syntheticpolypeptide"
LEPGEKPYKCPECGKSFSSKKHLAEHQRTHTPNPHRRTDPSHKPFQYKCPECGKSFSTTGALTEHQRTHTPNPHRRTDPSHKPFQYKCPECGKSFSREDNLHTHQRTHTHPRAPIPKPFQYKCPECGKSFSHKNALQNHQRTHTHPRAPIPKPFQYKCPECGKSFSHKNALQNHQRTHTGEKPYKCPECGKSFSRSDELVRHQRTHTGEKPYKCPECGKSFSTKNSLTEHQRTHTGKKTS

SEQ ID NO: 898 - protein, 251 aa, synthetic construct
  REGION 1..251: note = source = /note="Description of Artificial Sequence: Syntheticpolypeptide"
LEPGEKPYKCPECGKSFSTSGSLVRHQRTHTPNPHRRTDPSHKPFQYKCPECGKSFSSKKHLAEHQRTHTPNPHRRTDPSHKPFQYKCPECGKSFSTTGALTEHQRTHTPNPHRRTDPSHKPFQYKCPECGKSFSREDNLHTHQRTHTHPRAPIPKPFQYKCPECGKSFSHKNALQNHQRTHTHPRAPIPKPFQYKCPECGKSFSHKNALQNHQRTHTGEKPYKCPECGKSFSRSDELVRHQRTHTGKKTS

SEQ ID NO: 899 - protein, 232 aa, synthetic construct
  REGION 1..232: note = source = /note="Description of Artificial Sequence: Syntheticpolypeptide"
LEPGEKPYKCPECGKSFSREDNLHTHQRTHTHPRAPIPKPFQYKCPECGKSFSHKNALQNHQRTHTHPRAPIPKPFQYKCPECGKSFSHKNALQNHQRTHTGEKPYKCPECGKSFSRSDELVRHQRTHTHPRAPIPKPFQYKCPECGKSFSSKKHLAEHQRTHTGEKPYKCPECGKSFSRSDHLTTHQRTHTHPRAPIPKPFQYKCPECGKSFSRSDKLVRHQRTHTGKKTS

SEQ ID NO: 900 - protein, 225 aa, synthetic construct
  REGION 1..225: note = source = /note="Description of Artificial Sequence: Syntheticpolypeptide"
LEPGEKPYKCPECGKSFSREDNLHTHQRTHTHPRAPIPKPFQYKCPECGKSFSHKNALQNHQRTHTHPRAPIPKPFQYKCPECGKSFSHKNALQNHQRTHTGEKPYKCPECGKSFSRSDELVRHQRTHTHPRAPIPKPFQYKCPECGKSFSSKKHLAEHQRTHTGEKPYKCPECGKSFSRSDHLTTHQRTHTGEKPYKCPECGKSFSTSGHLVRHQRTHTGKKTS

SEQ ID NO: 901 - protein, 236 aa, synthetic construct
  REGION 1..236: note = source = /note="Description of Artificial Sequence: Syntheticpolypeptide"
LEPGEKPYKCPECGKSFSTTGALTEHQRTHTPNPHRRTDPSHKPFQYKCPECGKSFSREDNLHTHQRTHTHPRAPIPKPFQYKCPECGKSFSHKNALQNHQRTHTHPRAPIPKPFQYKCPECGKSFSHKNALQNHQRTHTGEKPYKCPECGKSFSRSDELVRHQRTHTHPRAPIPKPFQYKCPECGKSFSSKKHLAEHQRTHTGEKPYKCPECGKSFSRSDHLTTHQRTHTGKKTS

SEQ ID NO: 902 - protein, 247 aa, synthetic construct
  REGION 1..247: note = source = /note="Description of Artificial Sequence: Syntheticpolypeptide"
LEPGEKPYKCPECGKSFSSKKHLAEHQRTHTPNPHRRTDPSHKPFQYKCPECGKSFSTTGALTEHQRTHTPNPHRRTDPSHKPFQYKCPECGKSFSREDNLHTHQRTHTHPRAPIPKPFQYKCPECGKSFSHKNALQNHQRTHTHPRAPIPKPFQYKCPECGKSFSHKNALQNHQRTHTGEKPYKCPECGKSFSRSDELVRHQRTHTHPRAPIPKPFQYKCPECGKSFSSKKHLAEHQRTHTGKKTS

SEQ ID NO: 903 - protein, 233 aa, synthetic construct
  REGION 1..233: note = source = /note="Description of Artificial Sequence: Syntheticpolypeptide"
LEPGEKPYKCPECGKSFSQRANLRAHQRTHTPNPHRRTDPSHKPFQYKCPECGKSFSTSGSLVRHQRTHTPNPHRRTDPSHKPFQYKCPECGKSFSTTGALTEHQRTHTGEKPYKCPECGKSFSSPADLTRHQRTHTHPRAPIPKPFQYKCPECGKSFSQNSTLTEHQRTHTGEKPYKCPECGKSFSTHLDLIRHQRTHTGEKPYKCPECGKSFSQSGNLTEHQRTHTGKKTS

SEQ ID NO: 904 - protein, 233 aa, synthetic construct
  REGION 1..233: note = source = /note="Description of Artificial Sequence: Syntheticpolypeptide"
LEPGEKPYKCPECGKSFSDSGNLRVHQRTHTGEKPYKCPECGKSFSQRANLRAHQRTHTPNPHRRTDPSHKPFQYKCPECGKSFSTSGSLVRHQRTHTPNPHRRTDPSHKPFQYKCPECGKSFSTTGALTEHQRTHTGEKPYKCPECGKSFSSPADLTRHQRTHTHPRAPIPKPFQYKCPECGKSFSQNSTLTEHQRTHTGEKPYKCPECGKSFSTHLDLIRHQRTHTGKKTS

SEQ ID NO: 905 - protein, 240 aa, synthetic construct
  REGION 1..240: note = source = /note="Description of Artificial Sequence: Syntheticpolypeptide"
LEPGEKPYKCPECGKSFSRSDELVRHQRTHTHPRAPIPKPFQYKCPECGKSFSDSGNLRVHQRTHTGEKPYKCPECGKSFSQRANLRAHQRTHTPNPHRRTDPSHKPFQYKCPECGKSFSTSGSLVRHQRTHTPNPHRRTDPSHKPFQYKCPECGKSFSTTGALTEHQRTHTGEKPYKCPECGKSFSSPADLTRHQRTHTHPRAPIPKPFQYKCPECGKSFSQNSTLTEHQRTHTGKKTS

SEQ ID NO: 906 - protein, 244 aa, synthetic construct
  REGION 1..244: note = source = /note="Description of Artificial Sequence: Syntheticpolypeptide"
LEPGEKPYKCPECGKSFSDSGNLRVHQRTHTGEKPYKCPECGKSFSQRANLRAHQRTHTPNPHRRTDPSHKPFQYKCPECGKSFSTSGSLVRHQRTHTPNPHRRTDPSHKPFQYKCPECGKSFSTTGALTEHQRTHTGEKPYKCPECGKSFSSPADLTRHQRTHTHPRAPIPKPFQYKCPECGKSFSQNSTLTEHQRTHTPNPHRRTDPSHKPFQYKCPECGKSFSQRANLRAHQRTHTGKKTS

SEQ ID NO: 907 - protein, 240 aa, synthetic construct
  REGION 1..240: note = source = /note="Description of Artificial Sequence: Syntheticpolypeptide"
LEPGEKPYKCPECGKSFSDSGNLRVHQRTHTGEKPYKCPECGKSFSQRANLRAHQRTHTPNPHRRTDPSHKPFQYKCPECGKSFSTSGSLVRHQRTHTPNPHRRTDPSHKPFQYKCPECGKSFSTTGALTEHQRTHTGEKPYKCPECGKSFSSPADLTRHQRTHTHPRAPIPKPFQYKCPECGKSFSQNSTLTEHQRTHTHPRAPIPKPFQYKCPECGKSFSDSGNLRVHQRTHTGKKTS

SEQ ID NO: 908 - protein, 233 aa, synthetic construct
  REGION 1..233: note = source = /note="Description of Artificial Sequence: Syntheticpolypeptide"
LEPGEKPYKCPECGKSFSTSGSLVRHQRTHTGEKPYKCPECGKSFSRSDELVRHQRTHTHPRAPIPKPFQYKCPECGKSFSDSGNLRVHQRTHTGEKPYKCPECGKSFSQRANLRAHQRTHTPNPHRRTDPSHKPFQYKCPECGKSFSTSGSLVRHQRTHTPNPHRRTDPSHKPFQYKCPECGKSFSTTGALTEHQRTHTGEKPYKCPECGKSFSSPADLTRHQRTHTGKKTS

SEQ ID NO: 909 - protein, 226 aa, synthetic construct
  REGION 1..226: note = source = /note="Description of Artificial Sequence: Syntheticpolypeptide"
LEPGEKPYKCPECGKSFSRSDKLVRHQRTHTGEKPYKCPECGKSFSQAGHLASHQRTHTGEKPYKCPECGKSFSRSDHLTTHQRTHTGEKPYKCPECGKSFSRNDALTEHQRTHTPNPHRRTDPSHKPFQYKCPECGKSFSSKKALTEHQRTHTPNPHRRTDPSHKPFQYKCPECGKSFSTKNSLTEHQRTHTGEKPYKCPECGKSFSQKSSLIAHQRTHTGKKTS

SEQ ID NO: 910 - protein, 226 aa, synthetic construct
  REGION 1..226: note = source = /note="Description of Artificial Sequence: Syntheticpolypeptide"
LEPGEKPYKCPECGKSFSQSSNLVRHQRTHTGEKPYKCPECGKSFSRSDKLVRHQRTHTGEKPYKCPECGKSFSQAGHLASHQRTHTGEKPYKCPECGKSFSRSDHLTTHQRTHTGEKPYKCPECGKSFSRNDALTEHQRTHTPNPHRRTDPSHKPFQYKCPECGKSFSSKKALTEHQRTHTPNPHRRTDPSHKPFQYKCPECGKSFSTKNSLTEHQRTHTGKKTS

SEQ ID NO: 911 - protein, 215 aa, synthetic construct
  REGION 1..215: note = source = /note="Description of Artificial Sequence: Syntheticpolypeptide"
LEPGEKPYKCPECGKSFSQSSNLVRHQRTHTGEKPYKCPECGKSFSQSSNLVRHQRTHTGEKPYKCPECGKSFSRSDKLVRHQRTHTGEKPYKCPECGKSFSQAGHLASHQRTHTGEKPYKCPECGKSFSRSDHLTTHQRTHTGEKPYKCPECGKSFSRNDALTEHQRTHTPNPHRRTDPSHKPFQYKCPECGKSFSSKKALTEHQRTHTGKKTS

SEQ ID NO: 912 - protein, 211 aa, synthetic construct
  REGION 1..211: note = source = /note="Description of Artificial Sequence: Syntheticpolypeptide"
LEPGEKPYKCPECGKSFSQSSNLVRHQRTHTGEKPYKCPECGKSFSQSSNLVRHQRTHTGEKPYKCPECGKSFSRSDKLVRHQRTHTGEKPYKCPECGKSFSQAGHLASHQRTHTGEKPYKCPECGKSFSRSDHLTTHQRTHTGEKPYKCPECGKSFSRNDALTEHQRTHTHPRAPIPKPFQYKCPECGKSFSDKKDLTRHQRTHTGKKTS

SEQ ID NO: 913 - protein, 204 aa, synthetic construct
  REGION 1..204: note = source = /note="Description of Artificial Sequence: Syntheticpolypeptide"
LEPGEKPYKCPECGKSFSQSSNLVRHQRTHTGEKPYKCPECGKSFSQSSNLVRHQRTHTGEKPYKCPECGKSFSRSDKLVRHQRTHTGEKPYKCPECGKSFSQAGHLASHQRTHTGEKPYKCPECGKSFSRSDHLTTHQRTHTGEKPYKCPECGKSFSRNDALTEHQRTHTGEKPYKCPECGKSFSTKNSLTEHQRTHTGKKTS

SEQ ID NO: 914 - protein, 215 aa, synthetic construct
  REGION 1..215: note = source = /note="Description of Artificial Sequence: Syntheticpolypeptide"
LEPGEKPYKCPECGKSFSTSGNLTEHQRTHTPNPHRRTDPSHKPFQYKCPECGKSFSQSSNLVRHQRTHTGEKPYKCPECGKSFSQSSNLVRHQRTHTGEKPYKCPECGKSFSRSDKLVRHQRTHTGEKPYKCPECGKSFSQAGHLASHQRTHTGEKPYKCPECGKSFSRSDHLTTHQRTHTGEKPYKCPECGKSFSRNDALTEHQRTHTGKKTS

SEQ ID NO: 915 - protein, 215 aa, synthetic construct
  REGION 1..215: note = source = /note="Description of Artificial Sequence: Syntheticpolypeptide"
LEPGEKPYKCPECGKSFSHKNALQNHQRTHTGEKPYKCPECGKSFSTSGNLTEHQRTHTPNPHRRTDPSHKPFQYKCPECGKSFSQSSNLVRHQRTHTGEKPYKCPECGKSFSQSSNLVRHQRTHTGEKPYKCPECGKSFSRSDKLVRHQRTHTGEKPYKCPECGKSFSQAGHLASHQRTHTGEKPYKCPECGKSFSRSDHLTTHQRTHTGKKTS

SEQ ID NO: 916 - protein, 226 aa, synthetic construct
  REGION 1..226: note = source = /note="Description of Artificial Sequence: Syntheticpolypeptide"
LEPGEKPYKCPECGKSFSRADNLTEHQRTHTPNPHRRTDPSHKPFQYKCPECGKSFSHKNALQNHQRTHTGEKPYKCPECGKSFSTSGNLTEHQRTHTPNPHRRTDPSHKPFQYKCPECGKSFSQSSNLVRHQRTHTGEKPYKCPECGKSFSQSSNLVRHQRTHTGEKPYKCPECGKSFSRSDKLVRHQRTHTGEKPYKCPECGKSFSQAGHLASHQRTHTGKKTS

SEQ ID NO: 917 - protein, 226 aa, synthetic construct
  REGION 1..226: note = source = /note="Description of Artificial Sequence: Syntheticpolypeptide"
LEPGEKPYKCPECGKSFSRRDELNVHQRTHTGEKPYKCPECGKSFSRADNLTEHQRTHTPNPHRRTDPSHKPFQYKCPECGKSFSHKNALQNHQRTHTGEKPYKCPECGKSFSTSGNLTEHQRTHTPNPHRRTDPSHKPFQYKCPECGKSFSQSSNLVRHQRTHTGEKPYKCPECGKSFSQSSNLVRHQRTHTGEKPYKCPECGKSFSRSDKLVRHQRTHTGKKTS

SEQ ID NO: 918 - protein, 226 aa, synthetic construct
  REGION 1..226: note = source = /note="Description of Artificial Sequence: Syntheticpolypeptide"
LEPGEKPYKCPECGKSFSTHLDLIRHQRTHTGEKPYKCPECGKSFSRRDELNVHQRTHTGEKPYKCPECGKSFSRADNLTEHQRTHTPNPHRRTDPSHKPFQYKCPECGKSFSHKNALQNHQRTHTGEKPYKCPECGKSFSTSGNLTEHQRTHTPNPHRRTDPSHKPFQYKCPECGKSFSQSSNLVRHQRTHTGEKPYKCPECGKSFSQSSNLVRHQRTHTGKKTS

SEQ ID NO: 919 - protein, 226 aa, synthetic construct
  REGION 1..226: note = source = /note="Description of Artificial Sequence: Syntheticpolypeptide"
LEPGEKPYKCPECGKSFSSRRTCRAHQRTHTGEKPYKCPECGKSFSTHLDLIRHQRTHTGEKPYKCPECGKSFSRRDELNVHQRTHTGEKPYKCPECGKSFSRADNLTEHQRTHTPNPHRRTDPSHKPFQYKCPECGKSFSHKNALQNHQRTHTGEKPYKCPECGKSFSTSGNLTEHQRTHTPNPHRRTDPSHKPFQYKCPECGKSFSQSSNLVRHQRTHTGKKTS

SEQ ID NO: 920 - protein, 204 aa, synthetic construct
  REGION 1..204: note = source = /note="Description of Artificial Sequence: Syntheticpolypeptide"
LEPGEKPYKCPECGKSFSTSGNLTEHQRTHTGEKPYKCPECGKSFSTTGNLTVHQRTHTGEKPYKCPECGKSFSQLAHLRAHQRTHTGEKPYKCPECGKSFSQRAHLERHQRTHTGEKPYKCPECGKSFSRSDHLTNHQRTHTGEKPYKCPECGKSFSRSDELVRHQRTHTGEKPYKCPECGKSFSRSDELVRHQRTHTGKKTS

SEQ ID NO: 921 - protein, 204 aa, synthetic construct
  REGION 1..204: note = source = /note="Description of Artificial Sequence: Syntheticpolypeptide"
LEPGEKPYKCPECGKSFSHKNALQNHQRTHTGEKPYKCPECGKSFSTSGNLTEHQRTHTGEKPYKCPECGKSFSTTGNLTVHQRTHTGEKPYKCPECGKSFSQLAHLRAHQRTHTGEKPYKCPECGKSFSQRAHLERHQRTHTGEKPYKCPECGKSFSRSDHLTNHQRTHTGEKPYKCPECGKSFSRSDELVRHQRTHTGKKTS

SEQ ID NO: 922 - protein, 215 aa, synthetic construct
  REGION 1..215: note = source = /note="Description of Artificial Sequence: Syntheticpolypeptide"
LEPGEKPYKCPECGKSFSRADNLTEHQRTHTPNPHRRTDPSHKPFQYKCPECGKSFSHKNALQNHQRTHTGEKPYKCPECGKSFSTSGNLTEHQRTHTGEKPYKCPECGKSFSTTGNLTVHQRTHTGEKPYKCPECGKSFSQLAHLRAHQRTHTGEKPYKCPECGKSFSQRAHLERHQRTHTGEKPYKCPECGKSFSRSDHLTNHQRTHTGKKTS

SEQ ID NO: 923 - protein, 215 aa, synthetic construct
  REGION 1..215: note = source = /note="Description of Artificial Sequence: Syntheticpolypeptide"
LEPGEKPYKCPECGKSFSRRDELNVHQRTHTGEKPYKCPECGKSFSRADNLTEHQRTHTPNPHRRTDPSHKPFQYKCPECGKSFSHKNALQNHQRTHTGEKPYKCPECGKSFSTSGNLTEHQRTHTGEKPYKCPECGKSFSTTGNLTVHQRTHTGEKPYKCPECGKSFSQLAHLRAHQRTHTGEKPYKCPECGKSFSQRAHLERHQRTHTGKKTS

SEQ ID NO: 924 - protein, 215 aa, synthetic construct
  REGION 1..215: note = source = /note="Description of Artificial Sequence: Syntheticpolypeptide"
LEPGEKPYKCPECGKSFSTHLDLIRHQRTHTGEKPYKCPECGKSFSRRDELNVHQRTHTGEKPYKCPECGKSFSRADNLTEHQRTHTPNPHRRTDPSHKPFQYKCPECGKSFSHKNALQNHQRTHTGEKPYKCPECGKSFSTSGNLTEHQRTHTGEKPYKCPECGKSFSTTGNLTVHQRTHTGEKPYKCPECGKSFSQLAHLRAHQRTHTGKKTS

SEQ ID NO: 925 - protein, 215 aa, synthetic construct
  REGION 1..215: note = source = /note="Description of Artificial Sequence: Syntheticpolypeptide"
LEPGEKPYKCPECGKSFSSRRTCRAHQRTHTGEKPYKCPECGKSFSTHLDLIRHQRTHTGEKPYKCPECGKSFSRRDELNVHQRTHTGEKPYKCPECGKSFSRADNLTEHQRTHTPNPHRRTDPSHKPFQYKCPECGKSFSHKNALQNHQRTHTGEKPYKCPECGKSFSTSGNLTEHQRTHTGEKPYKCPECGKSFSTTGNLTVHQRTHTGKKTS

SEQ ID NO: 926 - protein, 215 aa, synthetic construct
  REGION 1..215: note = source = /note="Description of Artificial Sequence: Syntheticpolypeptide"
LEPGEKPYKCPECGKSFSQRANLRAHQRTHTGEKPYKCPECGKSFSSRRTCRAHQRTHTGEKPYKCPECGKSFSTHLDLIRHQRTHTGEKPYKCPECGKSFSRRDELNVHQRTHTGEKPYKCPECGKSFSRADNLTEHQRTHTPNPHRRTDPSHKPFQYKCPECGKSFSHKNALQNHQRTHTGEKPYKCPECGKSFSTSGNLTEHQRTHTGKKTS

SEQ ID NO: 927 - protein, 215 aa, synthetic construct
  REGION 1..215: note = source = /note="Description of Artificial Sequence: Syntheticpolypeptide"
LEPGEKPYKCPECGKSFSRSDKLVRHQRTHTGEKPYKCPECGKSFSQRANLRAHQRTHTGEKPYKCPECGKSFSSRRTCRAHQRTHTGEKPYKCPECGKSFSTHLDLIRHQRTHTGEKPYKCPECGKSFSRRDELNVHQRTHTGEKPYKCPECGKSFSRADNLTEHQRTHTPNPHRRTDPSHKPFQYKCPECGKSFSHKNALQNHQRTHTGKKTS

SEQ ID NO: 928 - protein, 211 aa, synthetic construct
  REGION 1..211: note = source = /note="Description of Artificial Sequence: Syntheticpolypeptide"
LEPGEKPYKCPECGKSFSHKNALQNHQRTHTGEKPYKCPECGKSFSTSGNLTEHQRTHTHPRAPIPKPFQYKCPECGKSFSQRANLRAHQRTHTGEKPYKCPECGKSFSRKDNLKNHQRTHTGEKPYKCPECGKSFSRSDKLVRHQRTHTGEKPYKCPECGKSFSRSDNLVRHQRTHTGEKPYKCPECGKSFSTSGHLVRHQRTHTGKKTS

SEQ ID NO: 929 - protein, 222 aa, synthetic construct
  REGION 1..222: note = source = /note="Description of Artificial Sequence: Syntheticpolypeptide"
LEPGEKPYKCPECGKSFSRADNLTEHQRTHTPNPHRRTDPSHKPFQYKCPECGKSFSHKNALQNHQRTHTGEKPYKCPECGKSFSTSGNLTEHQRTHTHPRAPIPKPFQYKCPECGKSFSQRANLRAHQRTHTGEKPYKCPECGKSFSRKDNLKNHQRTHTGEKPYKCPECGKSFSRSDKLVRHQRTHTGEKPYKCPECGKSFSRSDNLVRHQRTHTGKKTS

SEQ ID NO: 930 - protein, 222 aa, synthetic construct
  REGION 1..222: note = source = /note="Description of Artificial Sequence: Syntheticpolypeptide"
LEPGEKPYKCPECGKSFSRRDELNVHQRTHTGEKPYKCPECGKSFSRADNLTEHQRTHTPNPHRRTDPSHKPFQYKCPECGKSFSHKNALQNHQRTHTGEKPYKCPECGKSFSTSGNLTEHQRTHTHPRAPIPKPFQYKCPECGKSFSQRANLRAHQRTHTGEKPYKCPECGKSFSRKDNLKNHQRTHTGEKPYKCPECGKSFSRSDKLVRHQRTHTGKKTS

SEQ ID NO: 931 - protein, 222 aa, synthetic construct
  REGION 1..222: note = source = /note="Description of Artificial Sequence: Syntheticpolypeptide"
LEPGEKPYKCPECGKSFSTHLDLIRHQRTHTGEKPYKCPECGKSFSRRDELNVHQRTHTGEKPYKCPECGKSFSRADNLTEHQRTHTPNPHRRTDPSHKPFQYKCPECGKSFSHKNALQNHQRTHTGEKPYKCPECGKSFSTSGNLTEHQRTHTHPRAPIPKPFQYKCPECGKSFSQRANLRAHQRTHTGEKPYKCPECGKSFSRKDNLKNHQRTHTGKKTS

SEQ ID NO: 932 - protein, 222 aa, synthetic construct
  REGION 1..222: note = source = /note="Description of Artificial Sequence: Syntheticpolypeptide"
LEPGEKPYKCPECGKSFSSRRTCRAHQRTHTGEKPYKCPECGKSFSTHLDLIRHQRTHTGEKPYKCPECGKSFSRRDELNVHQRTHTGEKPYKCPECGKSFSRADNLTEHQRTHTPNPHRRTDPSHKPFQYKCPECGKSFSHKNALQNHQRTHTGEKPYKCPECGKSFSTSGNLTEHQRTHTHPRAPIPKPFQYKCPECGKSFSQRANLRAHQRTHTGKKTS

SEQ ID NO: 933 - protein, 204 aa, synthetic construct
  REGION 1..204: note = source = /note="Description of Artificial Sequence: Syntheticpolypeptide"
LEPGEKPYKCPECGKSFSQSGNLTEHQRTHTGEKPYKCPECGKSFSRSDKLVRHQRTHTGEKPYKCPECGKSFSQRANLRAHQRTHTGEKPYKCPECGKSFSSRRTCRAHQRTHTGEKPYKCPECGKSFSTHLDLIRHQRTHTGEKPYKCPECGKSFSRRDELNVHQRTHTGEKPYKCPECGKSFSRADNLTEHQRTHTGKKTS

SEQ ID NO: 934 - protein, 204 aa, synthetic construct
  REGION 1..204: note = source = /note="Description of Artificial Sequence: Syntheticpolypeptide"
LEPGEKPYKCPECGKSFSQLAHLRAHQRTHTGEKPYKCPECGKSFSQSGNLTEHQRTHTGEKPYKCPECGKSFSRSDKLVRHQRTHTGEKPYKCPECGKSFSQRANLRAHQRTHTGEKPYKCPECGKSFSSRRTCRAHQRTHTGEKPYKCPECGKSFSTHLDLIRHQRTHTGEKPYKCPECGKSFSRRDELNVHQRTHTGKKTS

SEQ ID NO: 935 - protein, 204 aa, synthetic construct
  REGION 1..204: note = source = /note="Description of Artificial Sequence: Syntheticpolypeptide"
LEPGEKPYKCPECGKSFSQRANLRAHQRTHTGEKPYKCPECGKSFSQLAHLRAHQRTHTGEKPYKCPECGKSFSQSGNLTEHQRTHTGEKPYKCPECGKSFSRSDKLVRHQRTHTGEKPYKCPECGKSFSQRANLRAHQRTHTGEKPYKCPECGKSFSSRRTCRAHQRTHTGEKPYKCPECGKSFSTHLDLIRHQRTHTGKKTS

SEQ ID NO: 936 - protein, 204 aa, synthetic construct
  REGION 1..204: note = source = /note="Description of Artificial Sequence: Syntheticpolypeptide"
LEPGEKPYKCPECGKSFSQRANLRAHQRTHTGEKPYKCPECGKSFSQRANLRAHQRTHTGEKPYKCPECGKSFSQLAHLRAHQRTHTGEKPYKCPECGKSFSQSGNLTEHQRTHTGEKPYKCPECGKSFSRSDKLVRHQRTHTGEKPYKCPECGKSFSQRANLRAHQRTHTGEKPYKCPECGKSFSSRRTCRAHQRTHTGKKTS

SEQ ID NO: 937 - protein, 204 aa, synthetic construct
  REGION 1..204: note = source = /note="Description of Artificial Sequence: Syntheticpolypeptide"
LEPGEKPYKCPECGKSFSSKKHLAEHQRTHTGEKPYKCPECGKSFSQRANLRAHQRTHTGEKPYKCPECGKSFSQRANLRAHQRTHTGEKPYKCPECGKSFSQLAHLRAHQRTHTGEKPYKCPECGKSFSQSGNLTEHQRTHTGEKPYKCPECGKSFSRSDKLVRHQRTHTGEKPYKCPECGKSFSQRANLRAHQRTHTGKKTS

SEQ ID NO: 938 - protein, 204 aa, synthetic construct
  REGION 1..204: note = source = /note="Description of Artificial Sequence: Syntheticpolypeptide"
LEPGEKPYKCPECGKSFSQRANLRAHQRTHTGEKPYKCPECGKSFSSKKHLAEHQRTHTGEKPYKCPECGKSFSQRANLRAHQRTHTGEKPYKCPECGKSFSQRANLRAHQRTHTGEKPYKCPECGKSFSQLAHLRAHQRTHTGEKPYKCPECGKSFSQSGNLTEHQRTHTGEKPYKCPECGKSFSRSDKLVRHQRTHTGKKTS

SEQ ID NO: 939 - protein, 204 aa, synthetic construct
  REGION 1..204: note = source = /note="Description of Artificial Sequence: Syntheticpolypeptide"
LEPGEKPYKCPECGKSFSHKNALQNHQRTHTGEKPYKCPECGKSFSQRANLRAHQRTHTGEKPYKCPECGKSFSSKKHLAEHQRTHTGEKPYKCPECGKSFSQRANLRAHQRTHTGEKPYKCPECGKSFSQRANLRAHQRTHTGEKPYKCPECGKSFSQLAHLRAHQRTHTGEKPYKCPECGKSFSQSGNLTEHQRTHTGKKTS

SEQ ID NO: 940 - protein, 211 aa, synthetic construct
  REGION 1..211: note = source = /note="Description of Artificial Sequence: Syntheticpolypeptide"
LEPGEKPYKCPECGKSFSQSGNLTEHQRTHTHPRAPIPKPFQYKCPECGKSFSHKNALQNHQRTHTGEKPYKCPECGKSFSQRANLRAHQRTHTGEKPYKCPECGKSFSSKKHLAEHQRTHTGEKPYKCPECGKSFSQRANLRAHQRTHTGEKPYKCPECGKSFSQRANLRAHQRTHTGEKPYKCPECGKSFSQLAHLRAHQRTHTGKKTS

SEQ ID NO: 941 - protein, 211 aa, synthetic construct
  REGION 1..211: note = source = /note="Description of Artificial Sequence: Syntheticpolypeptide"
LEPGEKPYKCPECGKSFSTTGNLTVHQRTHTGEKPYKCPECGKSFSQSGNLTEHQRTHTHPRAPIPKPFQYKCPECGKSFSHKNALQNHQRTHTGEKPYKCPECGKSFSQRANLRAHQRTHTGEKPYKCPECGKSFSSKKHLAEHQRTHTGEKPYKCPECGKSFSQRANLRAHQRTHTGEKPYKCPECGKSFSQRANLRAHQRTHTGKKTS

SEQ ID NO: 942 - protein, 222 aa, synthetic construct
  REGION 1..222: note = source = /note="Description of Artificial Sequence: Syntheticpolypeptide"
LEPGEKPYKCPECGKSFSRNDTLTEHQRTHTPNPHRRTDPSHKPFQYKCPECGKSFSTTGNLTVHQRTHTGEKPYKCPECGKSFSQSGNLTEHQRTHTHPRAPIPKPFQYKCPECGKSFSHKNALQNHQRTHTGEKPYKCPECGKSFSQRANLRAHQRTHTGEKPYKCPECGKSFSSKKHLAEHQRTHTGEKPYKCPECGKSFSQRANLRAHQRTHTGKKTS

SEQ ID NO: 943 - protein, 215 aa, synthetic construct
  REGION 1..215: note = source = /note="Description of Artificial Sequence: Syntheticpolypeptide"
LEPGEKPYKCPECGKSFSQLAHLRAHQRTHTGEKPYKCPECGKSFSQSGNLTEHQRTHTGEKPYKCPECGKSFSRSDKLVRHQRTHTGEKPYKCPECGKSFSQRANLRAHQRTHTGEKPYKCPECGKSFSSRRTCRAHQRTHTPNPHRRTDPSHKPFQYKCPECGKSFSQAGHLASHQRTHTGEKPYKCPECGKSFSQLAHLRAHQRTHTGKKTS

SEQ ID NO: 944 - protein, 215 aa, synthetic construct
  REGION 1..215: note = source = /note="Description of Artificial Sequence: Syntheticpolypeptide"
LEPGEKPYKCPECGKSFSQRANLRAHQRTHTGEKPYKCPECGKSFSQLAHLRAHQRTHTGEKPYKCPECGKSFSQSGNLTEHQRTHTGEKPYKCPECGKSFSRSDKLVRHQRTHTGEKPYKCPECGKSFSQRANLRAHQRTHTGEKPYKCPECGKSFSSRRTCRAHQRTHTPNPHRRTDPSHKPFQYKCPECGKSFSQAGHLASHQRTHTGKKTS

SEQ ID NO: 945 - protein, 211 aa, synthetic construct
  REGION 1..211: note = source = /note="Description of Artificial Sequence: Syntheticpolypeptide"
LEPGEKPYKCPECGKSFSQLAHLRAHQRTHTGEKPYKCPECGKSFSQSGNLTEHQRTHTGEKPYKCPECGKSFSRSDKLVRHQRTHTGEKPYKCPECGKSFSQRANLRAHQRTHTGEKPYKCPECGKSFSSRRTCRAHQRTHTHPRAPIPKPFQYKCPECGKSFSDPGNLVRHQRTHTGEKPYKCPECGKSFSTSGNLVRHQRTHTGKKTS

SEQ ID NO: 946 - protein, 211 aa, synthetic construct
  REGION 1..211: note = source = /note="Description of Artificial Sequence: Syntheticpolypeptide"
LEPGEKPYKCPECGKSFSQRANLRAHQRTHTGEKPYKCPECGKSFSQLAHLRAHQRTHTGEKPYKCPECGKSFSQSGNLTEHQRTHTGEKPYKCPECGKSFSRSDKLVRHQRTHTGEKPYKCPECGKSFSQRANLRAHQRTHTGEKPYKCPECGKSFSSRRTCRAHQRTHTHPRAPIPKPFQYKCPECGKSFSDPGNLVRHQRTHTGKKTS

SEQ ID NO: 947 - protein, 204 aa, synthetic construct
  REGION 1..204: note = source = /note="Description of Artificial Sequence: Syntheticpolypeptide"
LEPGEKPYKCPECGKSFSDSGNLRVHQRTHTGEKPYKCPECGKSFSQRANLRAHQRTHTGEKPYKCPECGKSFSQSSNLVRHQRTHTGEKPYKCPECGKSFSQRANLRAHQRTHTGEKPYKCPECGKSFSDPGHLVRHQRTHTGEKPYKCPECGKSFSRKDNLKNHQRTHTGEKPYKCPECGKSFSQSSSLVRHQRTHTGKKTS

SEQ ID NO: 948 - protein, 204 aa, synthetic construct
  REGION 1..204: note = source = /note="Description of Artificial Sequence: Syntheticpolypeptide"
LEPGEKPYKCPECGKSFSTSHSLTEHQRTHTGEKPYKCPECGKSFSDSGNLRVHQRTHTGEKPYKCPECGKSFSQRANLRAHQRTHTGEKPYKCPECGKSFSQSSNLVRHQRTHTGEKPYKCPECGKSFSQRANLRAHQRTHTGEKPYKCPECGKSFSDPGHLVRHQRTHTGEKPYKCPECGKSFSRKDNLKNHQRTHTGKKTS

SEQ ID NO: 949 - protein, 204 aa, synthetic construct
  REGION 1..204: note = source = /note="Description of Artificial Sequence: Syntheticpolypeptide"
LEPGEKPYKCPECGKSFSQRANLRAHQRTHTGEKPYKCPECGKSFSTSHSLTEHQRTHTGEKPYKCPECGKSFSDSGNLRVHQRTHTGEKPYKCPECGKSFSQRANLRAHQRTHTGEKPYKCPECGKSFSQSSNLVRHQRTHTGEKPYKCPECGKSFSQRANLRAHQRTHTGEKPYKCPECGKSFSDPGHLVRHQRTHTGKKTS

SEQ ID NO: 950 - protein, 204 aa, synthetic construct
  REGION 1..204: note = source = /note="Description of Artificial Sequence: Syntheticpolypeptide"
LEPGEKPYKCPECGKSFSQSGNLTEHQRTHTGEKPYKCPECGKSFSDKKDLTRHQRTHTGEKPYKCPECGKSFSQRANLRAHQRTHTGEKPYKCPECGKSFSQRANLRAHQRTHTGEKPYKCPECGKSFSRKDNLKNHQRTHTGEKPYKCPECGKSFSQSGDLRRHQRTHTGEKPYKCPECGKSFSRSDHLTNHQRTHTGKKTS

SEQ ID NO: 951 - protein, 204 aa, synthetic construct
  REGION 1..204: note = source = /note="Description of Artificial Sequence: Syntheticpolypeptide"
LEPGEKPYKCPECGKSFSTTGNLTVHQRTHTGEKPYKCPECGKSFSQSGNLTEHQRTHTGEKPYKCPECGKSFSDKKDLTRHQRTHTGEKPYKCPECGKSFSQRANLRAHQRTHTGEKPYKCPECGKSFSQRANLRAHQRTHTGEKPYKCPECGKSFSRKDNLKNHQRTHTGEKPYKCPECGKSFSQSGDLRRHQRTHTGKKTS

SEQ ID NO: 952 - protein, 215 aa, synthetic construct
  REGION 1..215: note = source = /note="Description of Artificial Sequence: Syntheticpolypeptide"
LEPGEKPYKCPECGKSFSQSGNLTEHQRTHTPNPHRRTDPSHKPFQYKCPECGKSFSTTGNLTVHQRTHTGEKPYKCPECGKSFSQSGNLTEHQRTHTGEKPYKCPECGKSFSDKKDLTRHQRTHTGEKPYKCPECGKSFSQRANLRAHQRTHTGEKPYKCPECGKSFSQRANLRAHQRTHTGEKPYKCPECGKSFSRKDNLKNHQRTHTGKKTS

SEQ ID NO: 953 - protein, 215 aa, synthetic construct
  REGION 1..215: note = source = /note="Description of Artificial Sequence: Syntheticpolypeptide"
LEPGEKPYKCPECGKSFSTTGNLTVHQRTHTGEKPYKCPECGKSFSQSGNLTEHQRTHTPNPHRRTDPSHKPFQYKCPECGKSFSTTGNLTVHQRTHTGEKPYKCPECGKSFSQSGNLTEHQRTHTGEKPYKCPECGKSFSDKKDLTRHQRTHTGEKPYKCPECGKSFSQRANLRAHQRTHTGEKPYKCPECGKSFSQRANLRAHQRTHTGKKTS

SEQ ID NO: 954 - protein, 226 aa, synthetic construct
  REGION 1..226: note = source = /note="Description of Artificial Sequence: Syntheticpolypeptide"
LEPGEKPYKCPECGKSFSRNDTLTEHQRTHTPNPHRRTDPSHKPFQYKCPECGKSFSTTGNLTVHQRTHTGEKPYKCPECGKSFSQSGNLTEHQRTHTPNPHRRTDPSHKPFQYKCPECGKSFSTTGNLTVHQRTHTGEKPYKCPECGKSFSQSGNLTEHQRTHTGEKPYKCPECGKSFSDKKDLTRHQRTHTGEKPYKCPECGKSFSQRANLRAHQRTHTGKKTS

SEQ ID NO: 955 - protein, 215 aa, synthetic construct
  REGION 1..215: note = source = /note="Description of Artificial Sequence: Syntheticpolypeptide"
LEPGEKPYKCPECGKSFSRSDNLVRHQRTHTGEKPYKCPECGKSFSRKDNLKNHQRTHTGEKPYKCPECGKSFSQSSNLVRHQRTHTGEKPYKCPECGKSFSQSGNLTEHQRTHTGEKPYKCPECGKSFSTTGNLTVHQRTHTGEKPYKCPECGKSFSTSGNLTEHQRTHTPNPHRRTDPSHKPFQYKCPECGKSFSHRTTLTNHQRTHTGKKTS

SEQ ID NO: 956 - protein, 211 aa, synthetic construct
  REGION 1..211: note = source = /note="Description of Artificial Sequence: Syntheticpolypeptide"
LEPGEKPYKCPECGKSFSRSDNLVRHQRTHTGEKPYKCPECGKSFSRKDNLKNHQRTHTGEKPYKCPECGKSFSQSSNLVRHQRTHTGEKPYKCPECGKSFSQSGNLTEHQRTHTGEKPYKCPECGKSFSTTGNLTVHQRTHTGEKPYKCPECGKSFSTSGNLTEHQRTHTHPRAPIPKPFQYKCPECGKSFSDPGALVRHQRTHTGKKTS

SEQ ID NO: 957 - protein, 204 aa, synthetic construct
  REGION 1..204: note = source = /note="Description of Artificial Sequence: Syntheticpolypeptide"
LEPGEKPYKCPECGKSFSQRANLRAHQRTHTGEKPYKCPECGKSFSRSDNLVRHQRTHTGEKPYKCPECGKSFSRKDNLKNHQRTHTGEKPYKCPECGKSFSQSSNLVRHQRTHTGEKPYKCPECGKSFSQSGNLTEHQRTHTGEKPYKCPECGKSFSTTGNLTVHQRTHTGEKPYKCPECGKSFSTSGNLTEHQRTHTGKKTS

SEQ ID NO: 958 - protein, 204 aa, synthetic construct
  REGION 1..204: note = source = /note="Description of Artificial Sequence: Syntheticpolypeptide"
LEPGEKPYKCPECGKSFSSKKALTEHQRTHTGEKPYKCPECGKSFSQRANLRAHQRTHTGEKPYKCPECGKSFSRSDNLVRHQRTHTGEKPYKCPECGKSFSRKDNLKNHQRTHTGEKPYKCPECGKSFSQSSNLVRHQRTHTGEKPYKCPECGKSFSQSGNLTEHQRTHTGEKPYKCPECGKSFSTTGNLTVHQRTHTGKKTS

SEQ ID NO: 959 - protein, 204 aa, synthetic construct
  REGION 1..204: note = source = /note="Description of Artificial Sequence: Syntheticpolypeptide"
LEPGEKPYKCPECGKSFSQLAHLRAHQRTHTGEKPYKCPECGKSFSSKKALTEHQRTHTGEKPYKCPECGKSFSQRANLRAHQRTHTGEKPYKCPECGKSFSRSDNLVRHQRTHTGEKPYKCPECGKSFSRKDNLKNHQRTHTGEKPYKCPECGKSFSQSSNLVRHQRTHTGEKPYKCPECGKSFSQSGNLTEHQRTHTGKKTS

SEQ ID NO: 960 - protein, 204 aa, synthetic construct
  REGION 1..204: note = source = /note="Description of Artificial Sequence: Syntheticpolypeptide"
LEPGEKPYKCPECGKSFSRSDNLVRHQRTHTGEKPYKCPECGKSFSQLAHLRAHQRTHTGEKPYKCPECGKSFSSKKALTEHQRTHTGEKPYKCPECGKSFSQRANLRAHQRTHTGEKPYKCPECGKSFSRSDNLVRHQRTHTGEKPYKCPECGKSFSRKDNLKNHQRTHTGEKPYKCPECGKSFSQSSNLVRHQRTHTGKKTS

SEQ ID NO: 961 - protein, 204 aa, synthetic construct
  REGION 1..204: note = source = /note="Description of Artificial Sequence: Syntheticpolypeptide"
LEPGEKPYKCPECGKSFSQLAHLRAHQRTHTGEKPYKCPECGKSFSRSDNLVRHQRTHTGEKPYKCPECGKSFSQLAHLRAHQRTHTGEKPYKCPECGKSFSSKKALTEHQRTHTGEKPYKCPECGKSFSQRANLRAHQRTHTGEKPYKCPECGKSFSRSDNLVRHQRTHTGEKPYKCPECGKSFSRKDNLKNHQRTHTGKKTS

SEQ ID NO: 962 - protein, 204 aa, synthetic construct
  REGION 1..204: note = source = /note="Description of Artificial Sequence: Syntheticpolypeptide"
LEPGEKPYKCPECGKSFSQSSNLVRHQRTHTGEKPYKCPECGKSFSQLAHLRAHQRTHTGEKPYKCPECGKSFSRSDNLVRHQRTHTGEKPYKCPECGKSFSQLAHLRAHQRTHTGEKPYKCPECGKSFSSKKALTEHQRTHTGEKPYKCPECGKSFSQRANLRAHQRTHTGEKPYKCPECGKSFSRSDNLVRHQRTHTGKKTS

SEQ ID NO: 963 - protein, 204 aa, synthetic construct
  REGION 1..204: note = source = /note="Description of Artificial Sequence: Syntheticpolypeptide"
LEPGEKPYKCPECGKSFSQRANLRAHQRTHTGEKPYKCPECGKSFSQSSNLVRHQRTHTGEKPYKCPECGKSFSQLAHLRAHQRTHTGEKPYKCPECGKSFSRSDNLVRHQRTHTGEKPYKCPECGKSFSQLAHLRAHQRTHTGEKPYKCPECGKSFSSKKALTEHQRTHTGEKPYKCPECGKSFSQRANLRAHQRTHTGKKTS

SEQ ID NO: 964 - protein, 204 aa, synthetic construct
  REGION 1..204: note = source = /note="Description of Artificial Sequence: Syntheticpolypeptide"
LEPGEKPYKCPECGKSFSQRANLRAHQRTHTGEKPYKCPECGKSFSQRANLRAHQRTHTGEKPYKCPECGKSFSQSSNLVRHQRTHTGEKPYKCPECGKSFSQLAHLRAHQRTHTGEKPYKCPECGKSFSRSDNLVRHQRTHTGEKPYKCPECGKSFSQLAHLRAHQRTHTGEKPYKCPECGKSFSSKKALTEHQRTHTGKKTS

SEQ ID NO: 965 - protein, 204 aa, synthetic construct
  REGION 1..204: note = source = /note="Description of Artificial Sequence: Syntheticpolypeptide"
LEPGEKPYKCPECGKSFSDSGNLRVHQRTHTGEKPYKCPECGKSFSQRANLRAHQRTHTGEKPYKCPECGKSFSQRANLRAHQRTHTGEKPYKCPECGKSFSQSSNLVRHQRTHTGEKPYKCPECGKSFSQLAHLRAHQRTHTGEKPYKCPECGKSFSRSDNLVRHQRTHTGEKPYKCPECGKSFSQLAHLRAHQRTHTGKKTS

SEQ ID NO: 966 - protein, 211 aa, synthetic construct
  REGION 1..211: note = source = /note="Description of Artificial Sequence: Syntheticpolypeptide"
LEPGEKPYKCPECGKSFSRRDELNVHQRTHTHPRAPIPKPFQYKCPECGKSFSDSGNLRVHQRTHTGEKPYKCPECGKSFSQRANLRAHQRTHTGEKPYKCPECGKSFSQRANLRAHQRTHTGEKPYKCPECGKSFSQSSNLVRHQRTHTGEKPYKCPECGKSFSQLAHLRAHQRTHTGEKPYKCPECGKSFSRSDNLVRHQRTHTGKKTS

SEQ ID NO: 967 - protein, 211 aa, synthetic construct
  REGION 1..211: note = source = /note="Description of Artificial Sequence: Syntheticpolypeptide"
LEPGEKPYKCPECGKSFSQLAHLRAHQRTHTGEKPYKCPECGKSFSRRDELNVHQRTHTHPRAPIPKPFQYKCPECGKSFSDSGNLRVHQRTHTGEKPYKCPECGKSFSQRANLRAHQRTHTGEKPYKCPECGKSFSQRANLRAHQRTHTGEKPYKCPECGKSFSQSSNLVRHQRTHTGEKPYKCPECGKSFSQLAHLRAHQRTHTGKKTS

SEQ ID NO: 968 - protein, 211 aa, synthetic construct
  REGION 1..211: note = source = /note="Description of Artificial Sequence: Syntheticpolypeptide"
LEPGEKPYKCPECGKSFSDKKDLTRHQRTHTGEKPYKCPECGKSFSQLAHLRAHQRTHTGEKPYKCPECGKSFSRRDELNVHQRTHTHPRAPIPKPFQYKCPECGKSFSDSGNLRVHQRTHTGEKPYKCPECGKSFSQRANLRAHQRTHTGEKPYKCPECGKSFSQRANLRAHQRTHTGEKPYKCPECGKSFSQSSNLVRHQRTHTGKKTS

SEQ ID NO: 969 - protein, 211 aa, synthetic construct
  REGION 1..211: note = source = /note="Description of Artificial Sequence: Syntheticpolypeptide"
LEPGEKPYKCPECGKSFSSKKHLAEHQRTHTGEKPYKCPECGKSFSDKKDLTRHQRTHTGEKPYKCPECGKSFSQLAHLRAHQRTHTGEKPYKCPECGKSFSRRDELNVHQRTHTHPRAPIPKPFQYKCPECGKSFSDSGNLRVHQRTHTGEKPYKCPECGKSFSQRANLRAHQRTHTGEKPYKCPECGKSFSQRANLRAHQRTHTGKKTS

SEQ ID NO: 970 - protein, 211 aa, synthetic construct
  REGION 1..211: note = source = /note="Description of Artificial Sequence: Syntheticpolypeptide"
LEPGEKPYKCPECGKSFSSKKHLAEHQRTHTGEKPYKCPECGKSFSSKKHLAEHQRTHTGEKPYKCPECGKSFSDKKDLTRHQRTHTGEKPYKCPECGKSFSQLAHLRAHQRTHTGEKPYKCPECGKSFSRRDELNVHQRTHTHPRAPIPKPFQYKCPECGKSFSDSGNLRVHQRTHTGEKPYKCPECGKSFSQRANLRAHQRTHTGKKTS

SEQ ID NO: 971 - protein, 211 aa, synthetic construct
  REGION 1..211: note = source = /note="Description of Artificial Sequence: Syntheticpolypeptide"
LEPGEKPYKCPECGKSFSSKKALTEHQRTHTGEKPYKCPECGKSFSSKKHLAEHQRTHTGEKPYKCPECGKSFSSKKHLAEHQRTHTGEKPYKCPECGKSFSDKKDLTRHQRTHTGEKPYKCPECGKSFSQLAHLRAHQRTHTGEKPYKCPECGKSFSRRDELNVHQRTHTHPRAPIPKPFQYKCPECGKSFSDSGNLRVHQRTHTGKKTS

SEQ ID NO: 972 - protein, 215 aa, synthetic construct
  REGION 1..215: note = source = /note="Description of Artificial Sequence: Syntheticpolypeptide"
LEPGEKPYKCPECGKSFSSKKALTEHQRTHTGEKPYKCPECGKSFSQRANLRAHQRTHTGEKPYKCPECGKSFSRSDNLVRHQRTHTGEKPYKCPECGKSFSRKDNLKNHQRTHTGEKPYKCPECGKSFSQSSNLVRHQRTHTGEKPYKCPECGKSFSQSGNLTEHQRTHTPNPHRRTDPSHKPFQYKCPECGKSFSQKSSLIAHQRTHTGKKTS

SEQ ID NO: 973 - protein, 204 aa, synthetic construct
  REGION 1..204: note = source = /note="Description of Artificial Sequence: Syntheticpolypeptide"
LEPGEKPYKCPECGKSFSRSDNLVRHQRTHTGEKPYKCPECGKSFSSPADLTRHQRTHTGEKPYKCPECGKSFSDSGNLRVHQRTHTGEKPYKCPECGKSFSQLAHLRAHQRTHTGEKPYKCPECGKSFSRSDHLTNHQRTHTGEKPYKCPECGKSFSQRANLRAHQRTHTGEKPYKCPECGKSFSRKDNLKNHQRTHTGKKTS

SEQ ID NO: 974 - protein, 204 aa, synthetic construct
  REGION 1..204: note = source = /note="Description of Artificial Sequence: Syntheticpolypeptide"
LEPGEKPYKCPECGKSFSQLAHLRAHQRTHTGEKPYKCPECGKSFSRSDNLVRHQRTHTGEKPYKCPECGKSFSSPADLTRHQRTHTGEKPYKCPECGKSFSDSGNLRVHQRTHTGEKPYKCPECGKSFSQLAHLRAHQRTHTGEKPYKCPECGKSFSRSDHLTNHQRTHTGEKPYKCPECGKSFSQRANLRAHQRTHTGKKTS

SEQ ID NO: 975 - protein, 204 aa, synthetic construct
  REGION 1..204: note = source = /note="Description of Artificial Sequence: Syntheticpolypeptide"
LEPGEKPYKCPECGKSFSRSDNLVRHQRTHTGEKPYKCPECGKSFSQLAHLRAHQRTHTGEKPYKCPECGKSFSRSDNLVRHQRTHTGEKPYKCPECGKSFSSPADLTRHQRTHTGEKPYKCPECGKSFSDSGNLRVHQRTHTGEKPYKCPECGKSFSQLAHLRAHQRTHTGEKPYKCPECGKSFSRSDHLTNHQRTHTGKKTS

SEQ ID NO: 976 - protein, 204 aa, synthetic construct
  REGION 1..204: note = source = /note="Description of Artificial Sequence: Syntheticpolypeptide"
LEPGEKPYKCPECGKSFSQRANLRAHQRTHTGEKPYKCPECGKSFSRSDNLVRHQRTHTGEKPYKCPECGKSFSQLAHLRAHQRTHTGEKPYKCPECGKSFSRSDNLVRHQRTHTGEKPYKCPECGKSFSSPADLTRHQRTHTGEKPYKCPECGKSFSDSGNLRVHQRTHTGEKPYKCPECGKSFSQLAHLRAHQRTHTGKKTS

SEQ ID NO: 977 - protein, 204 aa, synthetic construct
  REGION 1..204: note = source = /note="Description of Artificial Sequence: Syntheticpolypeptide"
LEPGEKPYKCPECGKSFSQRANLRAHQRTHTGEKPYKCPECGKSFSQRANLRAHQRTHTGEKPYKCPECGKSFSRSDNLVRHQRTHTGEKPYKCPECGKSFSQLAHLRAHQRTHTGEKPYKCPECGKSFSRSDNLVRHQRTHTGEKPYKCPECGKSFSSPADLTRHQRTHTGEKPYKCPECGKSFSDSGNLRVHQRTHTGKKTS

SEQ ID NO: 978 - protein, 204 aa, synthetic construct
  REGION 1..204: note = source = /note="Description of Artificial Sequence: Syntheticpolypeptide"
LEPGEKPYKCPECGKSFSQRANLRAHQRTHTGEKPYKCPECGKSFSQRANLRAHQRTHTGEKPYKCPECGKSFSQRANLRAHQRTHTGEKPYKCPECGKSFSRSDNLVRHQRTHTGEKPYKCPECGKSFSQLAHLRAHQRTHTGEKPYKCPECGKSFSRSDNLVRHQRTHTGEKPYKCPECGKSFSSPADLTRHQRTHTGKKTS

SEQ ID NO: 979 - protein, 204 aa, synthetic construct
  REGION 1..204: note = source = /note="Description of Artificial Sequence: Syntheticpolypeptide"
LEPGEKPYKCPECGKSFSTHLDLIRHQRTHTGEKPYKCPECGKSFSQRANLRAHQRTHTGEKPYKCPECGKSFSQRANLRAHQRTHTGEKPYKCPECGKSFSQRANLRAHQRTHTGEKPYKCPECGKSFSRSDNLVRHQRTHTGEKPYKCPECGKSFSQLAHLRAHQRTHTGEKPYKCPECGKSFSRSDNLVRHQRTHTGKKTS

SEQ ID NO: 980 - protein, 204 aa, synthetic construct
  REGION 1..204: note = source = /note="Description of Artificial Sequence: Syntheticpolypeptide"
LEPGEKPYKCPECGKSFSRRDELNVHQRTHTGEKPYKCPECGKSFSTHLDLIRHQRTHTGEKPYKCPECGKSFSQRANLRAHQRTHTGEKPYKCPECGKSFSQRANLRAHQRTHTGEKPYKCPECGKSFSQRANLRAHQRTHTGEKPYKCPECGKSFSRSDNLVRHQRTHTGEKPYKCPECGKSFSQLAHLRAHQRTHTGKKTS

SEQ ID NO: 981 - protein, 204 aa, synthetic construct
  REGION 1..204: note = source = /note="Description of Artificial Sequence: Syntheticpolypeptide"
LEPGEKPYKCPECGKSFSQLAHLRAHQRTHTGEKPYKCPECGKSFSRRDELNVHQRTHTGEKPYKCPECGKSFSTHLDLIRHQRTHTGEKPYKCPECGKSFSQRANLRAHQRTHTGEKPYKCPECGKSFSQRANLRAHQRTHTGEKPYKCPECGKSFSQRANLRAHQRTHTGEKPYKCPECGKSFSRSDNLVRHQRTHTGKKTS

SEQ ID NO: 982 - protein, 204 aa, synthetic construct
  REGION 1..204: note = source = /note="Description of Artificial Sequence: Syntheticpolypeptide"
LEPGEKPYKCPECGKSFSDKKDLTRHQRTHTGEKPYKCPECGKSFSQLAHLRAHQRTHTGEKPYKCPECGKSFSRRDELNVHQRTHTGEKPYKCPECGKSFSTHLDLIRHQRTHTGEKPYKCPECGKSFSQRANLRAHQRTHTGEKPYKCPECGKSFSQRANLRAHQRTHTGEKPYKCPECGKSFSQRANLRAHQRTHTGKKTS

SEQ ID NO: 983 - protein, 204 aa, synthetic construct
  REGION 1..204: note = source = /note="Description of Artificial Sequence: Syntheticpolypeptide"
LEPGEKPYKCPECGKSFSSKKHLAEHQRTHTGEKPYKCPECGKSFSDKKDLTRHQRTHTGEKPYKCPECGKSFSQLAHLRAHQRTHTGEKPYKCPECGKSFSRRDELNVHQRTHTGEKPYKCPECGKSFSTHLDLIRHQRTHTGEKPYKCPECGKSFSQRANLRAHQRTHTGEKPYKCPECGKSFSQRANLRAHQRTHTGKKTS

SEQ ID NO: 984 - protein, 204 aa, synthetic construct
  REGION 1..204: note = source = /note="Description of Artificial Sequence: Syntheticpolypeptide"
LEPGEKPYKCPECGKSFSSKKHLAEHQRTHTGEKPYKCPECGKSFSSKKHLAEHQRTHTGEKPYKCPECGKSFSDKKDLTRHQRTHTGEKPYKCPECGKSFSQLAHLRAHQRTHTGEKPYKCPECGKSFSRRDELNVHQRTHTGEKPYKCPECGKSFSTHLDLIRHQRTHTGEKPYKCPECGKSFSQRANLRAHQRTHTGKKTS

SEQ ID NO: 985 - protein, 204 aa, synthetic construct
  REGION 1..204: note = source = /note="Description of Artificial Sequence: Syntheticpolypeptide"
LEPGEKPYKCPECGKSFSSKKALTEHQRTHTGEKPYKCPECGKSFSSKKHLAEHQRTHTGEKPYKCPECGKSFSSKKHLAEHQRTHTGEKPYKCPECGKSFSDKKDLTRHQRTHTGEKPYKCPECGKSFSQLAHLRAHQRTHTGEKPYKCPECGKSFSRRDELNVHQRTHTGEKPYKCPECGKSFSTHLDLIRHQRTHTGKKTS

SEQ ID NO: 986 - protein, 204 aa, synthetic construct
  REGION 1..204: note = source = /note="Description of Artificial Sequence: Syntheticpolypeptide"
LEPGEKPYKCPECGKSFSSKKHLAEHQRTHTGEKPYKCPECGKSFSSKKALTEHQRTHTGEKPYKCPECGKSFSSKKHLAEHQRTHTGEKPYKCPECGKSFSSKKHLAEHQRTHTGEKPYKCPECGKSFSDKKDLTRHQRTHTGEKPYKCPECGKSFSQLAHLRAHQRTHTGEKPYKCPECGKSFSRRDELNVHQRTHTGKKTS

SEQ ID NO: 987 - protein, 204 aa, synthetic construct
  REGION 1..204: note = source = /note="Description of Artificial Sequence: Syntheticpolypeptide"
LEPGEKPYKCPECGKSFSRSDELVRHQRTHTGEKPYKCPECGKSFSSKKHLAEHQRTHTGEKPYKCPECGKSFSSKKALTEHQRTHTGEKPYKCPECGKSFSSKKHLAEHQRTHTGEKPYKCPECGKSFSSKKHLAEHQRTHTGEKPYKCPECGKSFSDKKDLTRHQRTHTGEKPYKCPECGKSFSQLAHLRAHQRTHTGKKTS

SEQ ID NO: 988 - protein, 204 aa, synthetic construct
  REGION 1..204: note = source = /note="Description of Artificial Sequence: Syntheticpolypeptide"
LEPGEKPYKCPECGKSFSHKNALQNHQRTHTGEKPYKCPECGKSFSRSDELVRHQRTHTGEKPYKCPECGKSFSSKKHLAEHQRTHTGEKPYKCPECGKSFSSKKALTEHQRTHTGEKPYKCPECGKSFSSKKHLAEHQRTHTGEKPYKCPECGKSFSSKKHLAEHQRTHTGEKPYKCPECGKSFSDKKDLTRHQRTHTGKKTS

SEQ ID NO: 989 - protein, 215 aa, synthetic construct
  REGION 1..215: note = source = /note="Description of Artificial Sequence: Syntheticpolypeptide"
LEPGEKPYKCPECGKSFSRADNLTEHQRTHTPNPHRRTDPSHKPFQYKCPECGKSFSHKNALQNHQRTHTGEKPYKCPECGKSFSRSDELVRHQRTHTGEKPYKCPECGKSFSSKKHLAEHQRTHTGEKPYKCPECGKSFSSKKALTEHQRTHTGEKPYKCPECGKSFSSKKHLAEHQRTHTGEKPYKCPECGKSFSSKKHLAEHQRTHTGKKTS

SEQ ID NO: 990 - protein, 215 aa, synthetic construct
  REGION 1..215: note = source = /note="Description of Artificial Sequence: Syntheticpolypeptide"
LEPGEKPYKCPECGKSFSHTGHLLEHQRTHTGEKPYKCPECGKSFSRADNLTEHQRTHTPNPHRRTDPSHKPFQYKCPECGKSFSHKNALQNHQRTHTGEKPYKCPECGKSFSRSDELVRHQRTHTGEKPYKCPECGKSFSSKKHLAEHQRTHTGEKPYKCPECGKSFSSKKALTEHQRTHTGEKPYKCPECGKSFSSKKHLAEHQRTHTGKKTS

SEQ ID NO: 991 - protein, 215 aa, synthetic construct
  REGION 1..215: note = source = /note="Description of Artificial Sequence: Syntheticpolypeptide"
LEPGEKPYKCPECGKSFSSPADLTRHQRTHTGEKPYKCPECGKSFSHTGHLLEHQRTHTGEKPYKCPECGKSFSRADNLTEHQRTHTPNPHRRTDPSHKPFQYKCPECGKSFSHKNALQNHQRTHTGEKPYKCPECGKSFSRSDELVRHQRTHTGEKPYKCPECGKSFSSKKHLAEHQRTHTGEKPYKCPECGKSFSSKKALTEHQRTHTGKKTS

SEQ ID NO: 992 - protein, 215 aa, synthetic construct
  REGION 1..215: note = source = /note="Description of Artificial Sequence: Syntheticpolypeptide"
LEPGEKPYKCPECGKSFSSKKALTEHQRTHTGEKPYKCPECGKSFSSPADLTRHQRTHTGEKPYKCPECGKSFSHTGHLLEHQRTHTGEKPYKCPECGKSFSRADNLTEHQRTHTPNPHRRTDPSHKPFQYKCPECGKSFSHKNALQNHQRTHTGEKPYKCPECGKSFSRSDELVRHQRTHTGEKPYKCPECGKSFSSKKHLAEHQRTHTGKKTS

SEQ ID NO: 993 - protein, 222 aa, synthetic construct
  REGION 1..222: note = source = /note="Description of Artificial Sequence: Syntheticpolypeptide"
LEPGEKPYKCPECGKSFSTSGNLTEHQRTHTHPRAPIPKPFQYKCPECGKSFSSKKALTEHQRTHTGEKPYKCPECGKSFSSPADLTRHQRTHTGEKPYKCPECGKSFSHTGHLLEHQRTHTGEKPYKCPECGKSFSRADNLTEHQRTHTPNPHRRTDPSHKPFQYKCPECGKSFSHKNALQNHQRTHTGEKPYKCPECGKSFSRSDELVRHQRTHTGKKTS

SEQ ID NO: 994 - protein, 222 aa, synthetic construct
  REGION 1..222: note = source = /note="Description of Artificial Sequence: Syntheticpolypeptide"
LEPGEKPYKCPECGKSFSQSGHLTEHQRTHTGEKPYKCPECGKSFSTSGNLTEHQRTHTHPRAPIPKPFQYKCPECGKSFSSKKALTEHQRTHTGEKPYKCPECGKSFSSPADLTRHQRTHTGEKPYKCPECGKSFSHTGHLLEHQRTHTGEKPYKCPECGKSFSRADNLTEHQRTHTPNPHRRTDPSHKPFQYKCPECGKSFSHKNALQNHQRTHTGKKTS

SEQ ID NO: 995 - protein, 204 aa, synthetic construct
  REGION 1..204: note = source = /note="Description of Artificial Sequence: Syntheticpolypeptide"
LEPGEKPYKCPECGKSFSQLAHLRAHQRTHTGEKPYKCPECGKSFSRADNLTEHQRTHTGEKPYKCPECGKSFSSPADLTRHQRTHTGEKPYKCPECGKSFSQSSNLVRHQRTHTGEKPYKCPECGKSFSQRAHLERHQRTHTGEKPYKCPECGKSFSQRANLRAHQRTHTGEKPYKCPECGKSFSQLAHLRAHQRTHTGKKTS

SEQ ID NO: 996 - protein, 204 aa, synthetic construct
  REGION 1..204: note = source = /note="Description of Artificial Sequence: Syntheticpolypeptide"
LEPGEKPYKCPECGKSFSRSDNLVRHQRTHTGEKPYKCPECGKSFSQLAHLRAHQRTHTGEKPYKCPECGKSFSRADNLTEHQRTHTGEKPYKCPECGKSFSSPADLTRHQRTHTGEKPYKCPECGKSFSQSSNLVRHQRTHTGEKPYKCPECGKSFSQRAHLERHQRTHTGEKPYKCPECGKSFSQRANLRAHQRTHTGKKTS

SEQ ID NO: 997 - protein, 204 aa, synthetic construct
  REGION 1..204: note = source = /note="Description of Artificial Sequence: Syntheticpolypeptide"
LEPGEKPYKCPECGKSFSQLAHLRAHQRTHTGEKPYKCPECGKSFSRSDNLVRHQRTHTGEKPYKCPECGKSFSQLAHLRAHQRTHTGEKPYKCPECGKSFSRADNLTEHQRTHTGEKPYKCPECGKSFSSPADLTRHQRTHTGEKPYKCPECGKSFSQSSNLVRHQRTHTGEKPYKCPECGKSFSQRAHLERHQRTHTGKKTS

SEQ ID NO: 998 - protein, 204 aa, synthetic construct
  REGION 1..204: note = source = /note="Description of Artificial Sequence: Syntheticpolypeptide"
LEPGEKPYKCPECGKSFSQRANLRAHQRTHTGEKPYKCPECGKSFSQLAHLRAHQRTHTGEKPYKCPECGKSFSRSDNLVRHQRTHTGEKPYKCPECGKSFSQLAHLRAHQRTHTGEKPYKCPECGKSFSRADNLTEHQRTHTGEKPYKCPECGKSFSSPADLTRHQRTHTGEKPYKCPECGKSFSQSSNLVRHQRTHTGKKTS

SEQ ID NO: 999 - protein, 204 aa, synthetic construct
  REGION 1..204: note = source = /note="Description of Artificial Sequence: Syntheticpolypeptide"
LEPGEKPYKCPECGKSFSQRANLRAHQRTHTGEKPYKCPECGKSFSQRANLRAHQRTHTGEKPYKCPECGKSFSQLAHLRAHQRTHTGEKPYKCPECGKSFSRSDNLVRHQRTHTGEKPYKCPECGKSFSQLAHLRAHQRTHTGEKPYKCPECGKSFSRADNLTEHQRTHTGEKPYKCPECGKSFSSPADLTRHQRTHTGKKTS

SEQ ID NO: 1000 - protein, 204 aa, synthetic construct
  REGION 1..204: note = source = /note="Description of Artificial Sequence: Syntheticpolypeptide"
LEPGEKPYKCPECGKSFSQRANLRAHQRTHTGEKPYKCPECGKSFSQRANLRAHQRTHTGEKPYKCPECGKSFSQRANLRAHQRTHTGEKPYKCPECGKSFSQLAHLRAHQRTHTGEKPYKCPECGKSFSRSDNLVRHQRTHTGEKPYKCPECGKSFSQLAHLRAHQRTHTGEKPYKCPECGKSFSRADNLTEHQRTHTGKKTS

SEQ ID NO: 1001 - protein, 204 aa, synthetic construct
  REGION 1..204: note = source = /note="Description of Artificial Sequence: Syntheticpolypeptide"
LEPGEKPYKCPECGKSFSQNSTLTEHQRTHTGEKPYKCPECGKSFSQRANLRAHQRTHTGEKPYKCPECGKSFSQRANLRAHQRTHTGEKPYKCPECGKSFSQRANLRAHQRTHTGEKPYKCPECGKSFSQLAHLRAHQRTHTGEKPYKCPECGKSFSRSDNLVRHQRTHTGEKPYKCPECGKSFSQLAHLRAHQRTHTGKKTS

SEQ ID NO: 1002 - protein, 204 aa, synthetic construct
  REGION 1..204: note = source = /note="Description of Artificial Sequence: Syntheticpolypeptide"
LEPGEKPYKCPECGKSFSQAGHLASHQRTHTGEKPYKCPECGKSFSQNSTLTEHQRTHTGEKPYKCPECGKSFSQRANLRAHQRTHTGEKPYKCPECGKSFSQRANLRAHQRTHTGEKPYKCPECGKSFSQRANLRAHQRTHTGEKPYKCPECGKSFSQLAHLRAHQRTHTGEKPYKCPECGKSFSRSDNLVRHQRTHTGKKTS

SEQ ID NO: 1003 - protein, 204 aa, synthetic construct
  REGION 1..204: note = source = /note="Description of Artificial Sequence: Syntheticpolypeptide"
LEPGEKPYKCPECGKSFSQSSNLVRHQRTHTGEKPYKCPECGKSFSQAGHLASHQRTHTGEKPYKCPECGKSFSQNSTLTEHQRTHTGEKPYKCPECGKSFSQRANLRAHQRTHTGEKPYKCPECGKSFSQRANLRAHQRTHTGEKPYKCPECGKSFSQRANLRAHQRTHTGEKPYKCPECGKSFSQLAHLRAHQRTHTGKKTS

SEQ ID NO: 1004 - protein, 204 aa, synthetic construct
  REGION 1..204: note = source = /note="Description of Artificial Sequence: Syntheticpolypeptide"
LEPGEKPYKCPECGKSFSSKKHLAEHQRTHTGEKPYKCPECGKSFSQSSNLVRHQRTHTGEKPYKCPECGKSFSQAGHLASHQRTHTGEKPYKCPECGKSFSQNSTLTEHQRTHTGEKPYKCPECGKSFSQRANLRAHQRTHTGEKPYKCPECGKSFSQRANLRAHQRTHTGEKPYKCPECGKSFSQRANLRAHQRTHTGKKTS

SEQ ID NO: 1005 - protein, 204 aa, synthetic construct
  REGION 1..204: note = source = /note="Description of Artificial Sequence: Syntheticpolypeptide"
LEPGEKPYKCPECGKSFSSKKHLAEHQRTHTGEKPYKCPECGKSFSSKKHLAEHQRTHTGEKPYKCPECGKSFSQSSNLVRHQRTHTGEKPYKCPECGKSFSQAGHLASHQRTHTGEKPYKCPECGKSFSQNSTLTEHQRTHTGEKPYKCPECGKSFSQRANLRAHQRTHTGEKPYKCPECGKSFSQRANLRAHQRTHTGKKTS

SEQ ID NO: 1006 - protein, 204 aa, synthetic construct
  REGION 1..204: note = source = /note="Description of Artificial Sequence: Syntheticpolypeptide"
LEPGEKPYKCPECGKSFSSKKHLAEHQRTHTGEKPYKCPECGKSFSSKKHLAEHQRTHTGEKPYKCPECGKSFSSKKHLAEHQRTHTGEKPYKCPECGKSFSQSSNLVRHQRTHTGEKPYKCPECGKSFSQAGHLASHQRTHTGEKPYKCPECGKSFSQNSTLTEHQRTHTGEKPYKCPECGKSFSQRANLRAHQRTHTGKKTS

SEQ ID NO: 1007 - protein, 204 aa, synthetic construct
  REGION 1..204: note = source = /note="Description of Artificial Sequence: Syntheticpolypeptide"
LEPGEKPYKCPECGKSFSDKKDLTRHQRTHTGEKPYKCPECGKSFSSKKHLAEHQRTHTGEKPYKCPECGKSFSSKKHLAEHQRTHTGEKPYKCPECGKSFSSKKHLAEHQRTHTGEKPYKCPECGKSFSQSSNLVRHQRTHTGEKPYKCPECGKSFSQAGHLASHQRTHTGEKPYKCPECGKSFSQNSTLTEHQRTHTGKKTS

SEQ ID NO: 1008 - protein, 204 aa, synthetic construct
  REGION 1..204: note = source = /note="Description of Artificial Sequence: Syntheticpolypeptide"
LEPGEKPYKCPECGKSFSRNDTLTEHQRTHTGEKPYKCPECGKSFSDKKDLTRHQRTHTGEKPYKCPECGKSFSSKKHLAEHQRTHTGEKPYKCPECGKSFSSKKHLAEHQRTHTGEKPYKCPECGKSFSSKKHLAEHQRTHTGEKPYKCPECGKSFSQSSNLVRHQRTHTGEKPYKCPECGKSFSQAGHLASHQRTHTGKKTS

SEQ ID NO: 1009 - protein, 204 aa, synthetic construct
  REGION 1..204: note = source = /note="Description of Artificial Sequence: Syntheticpolypeptide"
LEPGEKPYKCPECGKSFSQAGHLASHQRTHTGEKPYKCPECGKSFSRNDTLTEHQRTHTGEKPYKCPECGKSFSDKKDLTRHQRTHTGEKPYKCPECGKSFSSKKHLAEHQRTHTGEKPYKCPECGKSFSSKKHLAEHQRTHTGEKPYKCPECGKSFSSKKHLAEHQRTHTGEKPYKCPECGKSFSQSSNLVRHQRTHTGKKTS

SEQ ID NO: 1010 - protein, 204 aa, synthetic construct
  REGION 1..204: note = source = /note="Description of Artificial Sequence: Syntheticpolypeptide"
LEPGEKPYKCPECGKSFSSRRTCRAHQRTHTGEKPYKCPECGKSFSSKKHLAEHQRTHTGEKPYKCPECGKSFSTSHSLTEHQRTHTGEKPYKCPECGKSFSSKKHLAEHQRTHTGEKPYKCPECGKSFSSKKHLAEHQRTHTGEKPYKCPECGKSFSDSGNLRVHQRTHTGEKPYKCPECGKSFSRSDNLVRHQRTHTGKKTS

SEQ ID NO: 1011 - protein, 204 aa, synthetic construct
  REGION 1..204: note = source = /note="Description of Artificial Sequence: Syntheticpolypeptide"
LEPGEKPYKCPECGKSFSTSGNLVRHQRTHTGEKPYKCPECGKSFSSRRTCRAHQRTHTGEKPYKCPECGKSFSSKKHLAEHQRTHTGEKPYKCPECGKSFSTSHSLTEHQRTHTGEKPYKCPECGKSFSSKKHLAEHQRTHTGEKPYKCPECGKSFSSKKHLAEHQRTHTGEKPYKCPECGKSFSDSGNLRVHQRTHTGKKTS

SEQ ID NO: 1012 - protein, 211 aa, synthetic construct
  REGION 1..211: note = source = /note="Description of Artificial Sequence: Syntheticpolypeptide"
LEPGEKPYKCPECGKSFSQRANLRAHQRTHTGEKPYKCPECGKSFSQSGHLTEHQRTHTGEKPYKCPECGKSFSTSGNLTEHQRTHTHPRAPIPKPFQYKCPECGKSFSSKKALTEHQRTHTGEKPYKCPECGKSFSSPADLTRHQRTHTGEKPYKCPECGKSFSHTGHLLEHQRTHTGEKPYKCPECGKSFSRADNLTEHQRTHTGKKTS

SEQ ID NO: 1013 - protein, 211 aa, synthetic construct
  REGION 1..211: note = source = /note="Description of Artificial Sequence: Syntheticpolypeptide"
LEPGEKPYKCPECGKSFSQRANLRAHQRTHTGEKPYKCPECGKSFSQRANLRAHQRTHTGEKPYKCPECGKSFSQSGHLTEHQRTHTGEKPYKCPECGKSFSTSGNLTEHQRTHTHPRAPIPKPFQYKCPECGKSFSSKKALTEHQRTHTGEKPYKCPECGKSFSSPADLTRHQRTHTGEKPYKCPECGKSFSHTGHLLEHQRTHTGKKTS

SEQ ID NO: 1014 - protein, 222 aa, synthetic construct
  REGION 1..222: note = source = /note="Description of Artificial Sequence: Syntheticpolypeptide"
LEPGEKPYKCPECGKSFSRSDHLTTHQRTHTPNPHRRTDPSHKPFQYKCPECGKSFSQRANLRAHQRTHTGEKPYKCPECGKSFSQRANLRAHQRTHTGEKPYKCPECGKSFSQSGHLTEHQRTHTGEKPYKCPECGKSFSTSGNLTEHQRTHTHPRAPIPKPFQYKCPECGKSFSSKKALTEHQRTHTGEKPYKCPECGKSFSSPADLTRHQRTHTGKKTS

SEQ ID NO: 1015 - protein, 222 aa, synthetic construct
  REGION 1..222: note = source = /note="Description of Artificial Sequence: Syntheticpolypeptide"
LEPGEKPYKCPECGKSFSHKNALQNHQRTHTGEKPYKCPECGKSFSRSDHLTTHQRTHTPNPHRRTDPSHKPFQYKCPECGKSFSQRANLRAHQRTHTGEKPYKCPECGKSFSQRANLRAHQRTHTGEKPYKCPECGKSFSQSGHLTEHQRTHTGEKPYKCPECGKSFSTSGNLTEHQRTHTHPRAPIPKPFQYKCPECGKSFSSKKALTEHQRTHTGKKTS

SEQ ID NO: 1016 - protein, 204 aa, synthetic construct
  REGION 1..204: note = source = /note="Description of Artificial Sequence: Syntheticpolypeptide"
LEPGEKPYKCPECGKSFSQRANLRAHQRTHTGEKPYKCPECGKSFSQSGHLTEHQRTHTGEKPYKCPECGKSFSTSGNLTEHQRTHTGEKPYKCPECGKSFSTHLDLIRHQRTHTGEKPYKCPECGKSFSSKKALTEHQRTHTGEKPYKCPECGKSFSQSGDLRRHQRTHTGEKPYKCPECGKSFSERSHLREHQRTHTGKKTS

SEQ ID NO: 1017 - protein, 204 aa, synthetic construct
  REGION 1..204: note = source = /note="Description of Artificial Sequence: Syntheticpolypeptide"
LEPGEKPYKCPECGKSFSQRANLRAHQRTHTGEKPYKCPECGKSFSQRANLRAHQRTHTGEKPYKCPECGKSFSQSGHLTEHQRTHTGEKPYKCPECGKSFSTSGNLTEHQRTHTGEKPYKCPECGKSFSTHLDLIRHQRTHTGEKPYKCPECGKSFSSKKALTEHQRTHTGEKPYKCPECGKSFSQSGDLRRHQRTHTGKKTS

SEQ ID NO: 1018 - protein, 215 aa, synthetic construct
  REGION 1..215: note = source = /note="Description of Artificial Sequence: Syntheticpolypeptide"
LEPGEKPYKCPECGKSFSRSDHLTTHQRTHTPNPHRRTDPSHKPFQYKCPECGKSFSQRANLRAHQRTHTGEKPYKCPECGKSFSQRANLRAHQRTHTGEKPYKCPECGKSFSQSGHLTEHQRTHTGEKPYKCPECGKSFSTSGNLTEHQRTHTGEKPYKCPECGKSFSTHLDLIRHQRTHTGEKPYKCPECGKSFSSKKALTEHQRTHTGKKTS

SEQ ID NO: 1019 - protein, 215 aa, synthetic construct
  REGION 1..215: note = source = /note="Description of Artificial Sequence: Syntheticpolypeptide"
LEPGEKPYKCPECGKSFSHKNALQNHQRTHTGEKPYKCPECGKSFSRSDHLTTHQRTHTPNPHRRTDPSHKPFQYKCPECGKSFSQRANLRAHQRTHTGEKPYKCPECGKSFSQRANLRAHQRTHTGEKPYKCPECGKSFSQSGHLTEHQRTHTGEKPYKCPECGKSFSTSGNLTEHQRTHTGEKPYKCPECGKSFSTHLDLIRHQRTHTGKKTS

SEQ ID NO: 1020 - protein, 215 aa, synthetic construct
  REGION 1..215: note = source = /note="Description of Artificial Sequence: Syntheticpolypeptide"
LEPGEKPYKCPECGKSFSHKNALQNHQRTHTGEKPYKCPECGKSFSHKNALQNHQRTHTGEKPYKCPECGKSFSRSDHLTTHQRTHTPNPHRRTDPSHKPFQYKCPECGKSFSQRANLRAHQRTHTGEKPYKCPECGKSFSQRANLRAHQRTHTGEKPYKCPECGKSFSQSGHLTEHQRTHTGEKPYKCPECGKSFSTSGNLTEHQRTHTGKKTS

SEQ ID NO: 1021 - protein, 215 aa, synthetic construct
  REGION 1..215: note = source = /note="Description of Artificial Sequence: Syntheticpolypeptide"
LEPGEKPYKCPECGKSFSQLAHLRAHQRTHTGEKPYKCPECGKSFSHKNALQNHQRTHTGEKPYKCPECGKSFSHKNALQNHQRTHTGEKPYKCPECGKSFSRSDHLTTHQRTHTPNPHRRTDPSHKPFQYKCPECGKSFSQRANLRAHQRTHTGEKPYKCPECGKSFSQRANLRAHQRTHTGEKPYKCPECGKSFSQSGHLTEHQRTHTGKKTS

SEQ ID NO: 1022 - protein, 215 aa, synthetic construct
  REGION 1..215: note = source = /note="Description of Artificial Sequence: Syntheticpolypeptide"
LEPGEKPYKCPECGKSFSQLAHLRAHQRTHTGEKPYKCPECGKSFSQLAHLRAHQRTHTGEKPYKCPECGKSFSHKNALQNHQRTHTGEKPYKCPECGKSFSHKNALQNHQRTHTGEKPYKCPECGKSFSRSDHLTTHQRTHTPNPHRRTDPSHKPFQYKCPECGKSFSQRANLRAHQRTHTGEKPYKCPECGKSFSQRANLRAHQRTHTGKKTS

SEQ ID NO: 1023 - protein, 215 aa, synthetic construct
  REGION 1..215: note = source = /note="Description of Artificial Sequence: Syntheticpolypeptide"
LEPGEKPYKCPECGKSFSRSDNLVRHQRTHTGEKPYKCPECGKSFSQLAHLRAHQRTHTGEKPYKCPECGKSFSQLAHLRAHQRTHTGEKPYKCPECGKSFSHKNALQNHQRTHTGEKPYKCPECGKSFSHKNALQNHQRTHTGEKPYKCPECGKSFSRSDHLTTHQRTHTPNPHRRTDPSHKPFQYKCPECGKSFSQRANLRAHQRTHTGKKTS

SEQ ID NO: 1024 - protein, 215 aa, synthetic construct
  REGION 1..215: note = source = /note="Description of Artificial Sequence: Syntheticpolypeptide"
LEPGEKPYKCPECGKSFSQRANLRAHQRTHTGEKPYKCPECGKSFSQRANLRAHQRTHTGEKPYKCPECGKSFSQSGHLTEHQRTHTGEKPYKCPECGKSFSTSGNLTEHQRTHTPNPHRRTDPSHKPFQYKCPECGKSFSSPADLTRHQRTHTGEKPYKCPECGKSFSSKKALTEHQRTHTGEKPYKCPECGKSFSRSDDLVRHQRTHTGKKTS

SEQ ID NO: 1025 - protein, 226 aa, synthetic construct
  REGION 1..226: note = source = /note="Description of Artificial Sequence: Syntheticpolypeptide"
LEPGEKPYKCPECGKSFSRSDHLTTHQRTHTPNPHRRTDPSHKPFQYKCPECGKSFSQRANLRAHQRTHTGEKPYKCPECGKSFSQRANLRAHQRTHTGEKPYKCPECGKSFSQSGHLTEHQRTHTGEKPYKCPECGKSFSTSGNLTEHQRTHTPNPHRRTDPSHKPFQYKCPECGKSFSSPADLTRHQRTHTGEKPYKCPECGKSFSSKKALTEHQRTHTGKKTS

SEQ ID NO: 1026 - protein, 226 aa, synthetic construct
  REGION 1..226: note = source = /note="Description of Artificial Sequence: Syntheticpolypeptide"
LEPGEKPYKCPECGKSFSHKNALQNHQRTHTGEKPYKCPECGKSFSRSDHLTTHQRTHTPNPHRRTDPSHKPFQYKCPECGKSFSQRANLRAHQRTHTGEKPYKCPECGKSFSQRANLRAHQRTHTGEKPYKCPECGKSFSQSGHLTEHQRTHTGEKPYKCPECGKSFSTSGNLTEHQRTHTPNPHRRTDPSHKPFQYKCPECGKSFSSPADLTRHQRTHTGKKTS

SEQ ID NO: 1027 - protein, 211 aa, synthetic construct
  REGION 1..211: note = source = /note="Description of Artificial Sequence: Syntheticpolypeptide"
LEPGEKPYKCPECGKSFSQLAHLRAHQRTHTGEKPYKCPECGKSFSHKNALQNHQRTHTGEKPYKCPECGKSFSHKNALQNHQRTHTGEKPYKCPECGKSFSRSDHLTTHQRTHTHPRAPIPKPFQYKCPECGKSFSTTGNLTVHQRTHTGEKPYKCPECGKSFSQRANLRAHQRTHTGEKPYKCPECGKSFSQSSNLVRHQRTHTGKKTS

SEQ ID NO: 1028 - protein, 211 aa, synthetic construct
  REGION 1..211: note = source = /note="Description of Artificial Sequence: Syntheticpolypeptide"
LEPGEKPYKCPECGKSFSQLAHLRAHQRTHTGEKPYKCPECGKSFSQLAHLRAHQRTHTGEKPYKCPECGKSFSHKNALQNHQRTHTGEKPYKCPECGKSFSHKNALQNHQRTHTGEKPYKCPECGKSFSRSDHLTTHQRTHTHPRAPIPKPFQYKCPECGKSFSTTGNLTVHQRTHTGEKPYKCPECGKSFSQRANLRAHQRTHTGKKTS

SEQ ID NO: 1029 - protein, 211 aa, synthetic construct
  REGION 1..211: note = source = /note="Description of Artificial Sequence: Syntheticpolypeptide"
LEPGEKPYKCPECGKSFSRSDNLVRHQRTHTGEKPYKCPECGKSFSQLAHLRAHQRTHTGEKPYKCPECGKSFSQLAHLRAHQRTHTGEKPYKCPECGKSFSHKNALQNHQRTHTGEKPYKCPECGKSFSHKNALQNHQRTHTGEKPYKCPECGKSFSRSDHLTTHQRTHTHPRAPIPKPFQYKCPECGKSFSTTGNLTVHQRTHTGKKTS

SEQ ID NO: 1030 - protein, 204 aa, synthetic construct
  REGION 1..204: note = source = /note="Description of Artificial Sequence: Syntheticpolypeptide"
LEPGEKPYKCPECGKSFSQLAHLRAHQRTHTGEKPYKCPECGKSFSHKNALQNHQRTHTGEKPYKCPECGKSFSHKNALQNHQRTHTGEKPYKCPECGKSFSRSDHLTTHQRTHTGEKPYKCPECGKSFSHKNALQNHQRTHTGEKPYKCPECGKSFSQRANLRAHQRTHTGEKPYKCPECGKSFSQRANLRAHQRTHTGKKTS

SEQ ID NO: 1031 - protein, 204 aa, synthetic construct
  REGION 1..204: note = source = /note="Description of Artificial Sequence: Syntheticpolypeptide"
LEPGEKPYKCPECGKSFSQLAHLRAHQRTHTGEKPYKCPECGKSFSQLAHLRAHQRTHTGEKPYKCPECGKSFSHKNALQNHQRTHTGEKPYKCPECGKSFSHKNALQNHQRTHTGEKPYKCPECGKSFSRSDHLTTHQRTHTGEKPYKCPECGKSFSHKNALQNHQRTHTGEKPYKCPECGKSFSQRANLRAHQRTHTGKKTS

SEQ ID NO: 1032 - protein, 204 aa, synthetic construct
  REGION 1..204: note = source = /note="Description of Artificial Sequence: Syntheticpolypeptide"
LEPGEKPYKCPECGKSFSRSDNLVRHQRTHTGEKPYKCPECGKSFSQLAHLRAHQRTHTGEKPYKCPECGKSFSQLAHLRAHQRTHTGEKPYKCPECGKSFSHKNALQNHQRTHTGEKPYKCPECGKSFSHKNALQNHQRTHTGEKPYKCPECGKSFSRSDHLTTHQRTHTGEKPYKCPECGKSFSHKNALQNHQRTHTGKKTS

SEQ ID NO: 1033 - protein, 204 aa, synthetic construct
  REGION 1..204: note = source = /note="Description of Artificial Sequence: Syntheticpolypeptide"
LEPGEKPYKCPECGKSFSRSDHLTNHQRTHTGEKPYKCPECGKSFSRSDNLVRHQRTHTGEKPYKCPECGKSFSQLAHLRAHQRTHTGEKPYKCPECGKSFSQLAHLRAHQRTHTGEKPYKCPECGKSFSHKNALQNHQRTHTGEKPYKCPECGKSFSHKNALQNHQRTHTGEKPYKCPECGKSFSRSDHLTTHQRTHTGKKTS

SEQ ID NO: 1034 - protein, 211 aa, synthetic construct
  REGION 1..211: note = source = /note="Description of Artificial Sequence: Syntheticpolypeptide"
LEPGEKPYKCPECGKSFSRNDALTEHQRTHTHPRAPIPKPFQYKCPECGKSFSRSDHLTNHQRTHTGEKPYKCPECGKSFSRSDNLVRHQRTHTGEKPYKCPECGKSFSQLAHLRAHQRTHTGEKPYKCPECGKSFSQLAHLRAHQRTHTGEKPYKCPECGKSFSHKNALQNHQRTHTGEKPYKCPECGKSFSHKNALQNHQRTHTGKKTS

SEQ ID NO: 1035 - protein, 211 aa, synthetic construct
  REGION 1..211: note = source = /note="Description of Artificial Sequence: Syntheticpolypeptide"
LEPGEKPYKCPECGKSFSRSDKLTEHQRTHTGEKPYKCPECGKSFSRNDALTEHQRTHTHPRAPIPKPFQYKCPECGKSFSRSDHLTNHQRTHTGEKPYKCPECGKSFSRSDNLVRHQRTHTGEKPYKCPECGKSFSQLAHLRAHQRTHTGEKPYKCPECGKSFSQLAHLRAHQRTHTGEKPYKCPECGKSFSHKNALQNHQRTHTGKKTS

SEQ ID NO: 1036 - protein, 211 aa, synthetic construct
  REGION 1..211: note = source = /note="Description of Artificial Sequence: Syntheticpolypeptide"
LEPGEKPYKCPECGKSFSTTGALTEHQRTHTGEKPYKCPECGKSFSRSDKLTEHQRTHTGEKPYKCPECGKSFSRNDALTEHQRTHTHPRAPIPKPFQYKCPECGKSFSRSDHLTNHQRTHTGEKPYKCPECGKSFSRSDNLVRHQRTHTGEKPYKCPECGKSFSQLAHLRAHQRTHTGEKPYKCPECGKSFSQLAHLRAHQRTHTGKKTS

SEQ ID NO: 1037 - protein, 211 aa, synthetic construct
  REGION 1..211: note = source = /note="Description of Artificial Sequence: Syntheticpolypeptide"
LEPGEKPYKCPECGKSFSTSHSLTEHQRTHTGEKPYKCPECGKSFSTTGALTEHQRTHTGEKPYKCPECGKSFSRSDKLTEHQRTHTGEKPYKCPECGKSFSRNDALTEHQRTHTHPRAPIPKPFQYKCPECGKSFSRSDHLTNHQRTHTGEKPYKCPECGKSFSRSDNLVRHQRTHTGEKPYKCPECGKSFSQLAHLRAHQRTHTGKKTS

SEQ ID NO: 1038 - protein, 211 aa, synthetic construct
  REGION 1..211: note = source = /note="Description of Artificial Sequence: Syntheticpolypeptide"
LEPGEKPYKCPECGKSFSSKKALTEHQRTHTGEKPYKCPECGKSFSTSHSLTEHQRTHTGEKPYKCPECGKSFSTTGALTEHQRTHTGEKPYKCPECGKSFSRSDKLTEHQRTHTGEKPYKCPECGKSFSRNDALTEHQRTHTHPRAPIPKPFQYKCPECGKSFSRSDHLTNHQRTHTGEKPYKCPECGKSFSRSDNLVRHQRTHTGKKTS

SEQ ID NO: 1039 - protein, 211 aa, synthetic construct
  REGION 1..211: note = source = /note="Description of Artificial Sequence: Syntheticpolypeptide"
LEPGEKPYKCPECGKSFSSRRTCRAHQRTHTGEKPYKCPECGKSFSSKKALTEHQRTHTGEKPYKCPECGKSFSTSHSLTEHQRTHTGEKPYKCPECGKSFSTTGALTEHQRTHTGEKPYKCPECGKSFSRSDKLTEHQRTHTGEKPYKCPECGKSFSRNDALTEHQRTHTHPRAPIPKPFQYKCPECGKSFSRSDHLTNHQRTHTGKKTS

SEQ ID NO: 1040 - protein, 222 aa, synthetic construct
  REGION 1..222: note = source = /note="Description of Artificial Sequence: Syntheticpolypeptide"
LEPGEKPYKCPECGKSFSRNDALTEHQRTHTHPRAPIPKPFQYKCPECGKSFSRSDHLTNHQRTHTGEKPYKCPECGKSFSRSDNLVRHQRTHTGEKPYKCPECGKSFSQLAHLRAHQRTHTGEKPYKCPECGKSFSQLAHLRAHQRTHTGEKPYKCPECGKSFSHKNALQNHQRTHTPNPHRRTDPSHKPFQYKCPECGKSFSQRAHLERHQRTHTGKKTS

SEQ ID NO: 1041 - protein, 218 aa, synthetic construct
  REGION 1..218: note = source = /note="Description of Artificial Sequence: Syntheticpolypeptide"
LEPGEKPYKCPECGKSFSRNDALTEHQRTHTHPRAPIPKPFQYKCPECGKSFSRSDHLTNHQRTHTGEKPYKCPECGKSFSRSDNLVRHQRTHTGEKPYKCPECGKSFSQLAHLRAHQRTHTGEKPYKCPECGKSFSQLAHLRAHQRTHTGEKPYKCPECGKSFSHKNALQNHQRTHTHPRAPIPKPFQYKCPECGKSFSTSGNLVRHQRTHTGKKTS

SEQ ID NO: 1042 - protein, 211 aa, synthetic construct
  REGION 1..211: note = source = /note="Description of Artificial Sequence: Syntheticpolypeptide"
LEPGEKPYKCPECGKSFSTSGELVRHQRTHTGEKPYKCPECGKSFSRSDDLVRHQRTHTHPRAPIPKPFQYKCPECGKSFSDPGALVRHQRTHTGEKPYKCPECGKSFSRSDNLVRHQRTHTGEKPYKCPECGKSFSQLAHLRAHQRTHTGEKPYKCPECGKSFSRKDNLKNHQRTHTGEKPYKCPECGKSFSREDNLHTHQRTHTGKKTS

SEQ ID NO: 1043 - protein, 218 aa, synthetic construct
  REGION 1..218: note = source = /note="Description of Artificial Sequence: Syntheticpolypeptide"
LEPGEKPYKCPECGKSFSTSHSLTEHQRTHTHPRAPIPKPFQYKCPECGKSFSTSGELVRHQRTHTGEKPYKCPECGKSFSRSDDLVRHQRTHTHPRAPIPKPFQYKCPECGKSFSDPGALVRHQRTHTGEKPYKCPECGKSFSRSDNLVRHQRTHTGEKPYKCPECGKSFSQLAHLRAHQRTHTGEKPYKCPECGKSFSRKDNLKNHQRTHTGKKTS

SEQ ID NO: 1044 - protein, 218 aa, synthetic construct
  REGION 1..218: note = source = /note="Description of Artificial Sequence: Syntheticpolypeptide"
LEPGEKPYKCPECGKSFSSKKALTEHQRTHTGEKPYKCPECGKSFSTSHSLTEHQRTHTHPRAPIPKPFQYKCPECGKSFSTSGELVRHQRTHTGEKPYKCPECGKSFSRSDDLVRHQRTHTHPRAPIPKPFQYKCPECGKSFSDPGALVRHQRTHTGEKPYKCPECGKSFSRSDNLVRHQRTHTGEKPYKCPECGKSFSQLAHLRAHQRTHTGKKTS

SEQ ID NO: 1045 - protein, 218 aa, synthetic construct
  REGION 1..218: note = source = /note="Description of Artificial Sequence: Syntheticpolypeptide"
LEPGEKPYKCPECGKSFSSRRTCRAHQRTHTGEKPYKCPECGKSFSSKKALTEHQRTHTGEKPYKCPECGKSFSTSHSLTEHQRTHTHPRAPIPKPFQYKCPECGKSFSTSGELVRHQRTHTGEKPYKCPECGKSFSRSDDLVRHQRTHTHPRAPIPKPFQYKCPECGKSFSDPGALVRHQRTHTGEKPYKCPECGKSFSRSDNLVRHQRTHTGKKTS

SEQ ID NO: 1046 - protein, 218 aa, synthetic construct
  REGION 1..218: note = source = /note="Description of Artificial Sequence: Syntheticpolypeptide"
LEPGEKPYKCPECGKSFSSPADLTRHQRTHTGEKPYKCPECGKSFSSRRTCRAHQRTHTGEKPYKCPECGKSFSSKKALTEHQRTHTGEKPYKCPECGKSFSTSHSLTEHQRTHTHPRAPIPKPFQYKCPECGKSFSTSGELVRHQRTHTGEKPYKCPECGKSFSRSDDLVRHQRTHTHPRAPIPKPFQYKCPECGKSFSDPGALVRHQRTHTGKKTS

SEQ ID NO: 1047 - protein, 204 aa, synthetic construct
  REGION 1..204: note = source = /note="Description of Artificial Sequence: Syntheticpolypeptide"
LEPGEKPYKCPECGKSFSTTGALTEHQRTHTGEKPYKCPECGKSFSRSDKLTEHQRTHTGEKPYKCPECGKSFSRNDALTEHQRTHTGEKPYKCPECGKSFSTSGHLVRHQRTHTGEKPYKCPECGKSFSQLAHLRAHQRTHTGEKPYKCPECGKSFSRSDNLVRHQRTHTGEKPYKCPECGKSFSQSSNLVRHQRTHTGKKTS

SEQ ID NO: 1048 - protein, 204 aa, synthetic construct
  REGION 1..204: note = source = /note="Description of Artificial Sequence: Syntheticpolypeptide"
LEPGEKPYKCPECGKSFSTSHSLTEHQRTHTGEKPYKCPECGKSFSTTGALTEHQRTHTGEKPYKCPECGKSFSRSDKLTEHQRTHTGEKPYKCPECGKSFSRNDALTEHQRTHTGEKPYKCPECGKSFSTSGHLVRHQRTHTGEKPYKCPECGKSFSQLAHLRAHQRTHTGEKPYKCPECGKSFSRSDNLVRHQRTHTGKKTS

SEQ ID NO: 1049 - protein, 204 aa, synthetic construct
  REGION 1..204: note = source = /note="Description of Artificial Sequence: Syntheticpolypeptide"
LEPGEKPYKCPECGKSFSSKKALTEHQRTHTGEKPYKCPECGKSFSTSHSLTEHQRTHTGEKPYKCPECGKSFSTTGALTEHQRTHTGEKPYKCPECGKSFSRSDKLTEHQRTHTGEKPYKCPECGKSFSRNDALTEHQRTHTGEKPYKCPECGKSFSTSGHLVRHQRTHTGEKPYKCPECGKSFSQLAHLRAHQRTHTGKKTS

SEQ ID NO: 1050 - protein, 204 aa, synthetic construct
  REGION 1..204: note = source = /note="Description of Artificial Sequence: Syntheticpolypeptide"
LEPGEKPYKCPECGKSFSSRRTCRAHQRTHTGEKPYKCPECGKSFSSKKALTEHQRTHTGEKPYKCPECGKSFSTSHSLTEHQRTHTGEKPYKCPECGKSFSTTGALTEHQRTHTGEKPYKCPECGKSFSRSDKLTEHQRTHTGEKPYKCPECGKSFSRNDALTEHQRTHTGEKPYKCPECGKSFSTSGHLVRHQRTHTGKKTS

SEQ ID NO: 1051 - protein, 204 aa, synthetic construct
  REGION 1..204: note = source = /note="Description of Artificial Sequence: Syntheticpolypeptide"
LEPGEKPYKCPECGKSFSSPADLTRHQRTHTGEKPYKCPECGKSFSSRRTCRAHQRTHTGEKPYKCPECGKSFSSKKALTEHQRTHTGEKPYKCPECGKSFSTSHSLTEHQRTHTGEKPYKCPECGKSFSTTGALTEHQRTHTGEKPYKCPECGKSFSRSDKLTEHQRTHTGEKPYKCPECGKSFSRNDALTEHQRTHTGKKTS

SEQ ID NO: 1052 - protein, 204 aa, synthetic construct
  REGION 1..204: note = source = /note="Description of Artificial Sequence: Syntheticpolypeptide"
LEPGEKPYKCPECGKSFSRSDDLVRHQRTHTGEKPYKCPECGKSFSSPADLTRHQRTHTGEKPYKCPECGKSFSSRRTCRAHQRTHTGEKPYKCPECGKSFSSKKALTEHQRTHTGEKPYKCPECGKSFSTSHSLTEHQRTHTGEKPYKCPECGKSFSTTGALTEHQRTHTGEKPYKCPECGKSFSRSDKLTEHQRTHTGKKTS

SEQ ID NO: 1053 - protein, 204 aa, synthetic construct
  REGION 1..204: note = source = /note="Description of Artificial Sequence: Syntheticpolypeptide"
LEPGEKPYKCPECGKSFSRSDHLTTHQRTHTGEKPYKCPECGKSFSRSDDLVRHQRTHTGEKPYKCPECGKSFSSPADLTRHQRTHTGEKPYKCPECGKSFSSRRTCRAHQRTHTGEKPYKCPECGKSFSSKKALTEHQRTHTGEKPYKCPECGKSFSTSHSLTEHQRTHTGEKPYKCPECGKSFSTTGALTEHQRTHTGKKTS

SEQ ID NO: 1054 - protein, 211 aa, synthetic construct
  REGION 1..211: note = source = /note="Description of Artificial Sequence: Syntheticpolypeptide"
LEPGEKPYKCPECGKSFSRSDDLVRHQRTHTGEKPYKCPECGKSFSSPADLTRHQRTHTGEKPYKCPECGKSFSSRRTCRAHQRTHTGEKPYKCPECGKSFSSKKALTEHQRTHTGEKPYKCPECGKSFSTSHSLTEHQRTHTHPRAPIPKPFQYKCPECGKSFSTSGELVRHQRTHTGEKPYKCPECGKSFSRSDDLVRHQRTHTGKKTS

SEQ ID NO: 1055 - protein, 211 aa, synthetic construct
  REGION 1..211: note = source = /note="Description of Artificial Sequence: Syntheticpolypeptide"
LEPGEKPYKCPECGKSFSRSDHLTTHQRTHTGEKPYKCPECGKSFSRSDDLVRHQRTHTGEKPYKCPECGKSFSSPADLTRHQRTHTGEKPYKCPECGKSFSSRRTCRAHQRTHTGEKPYKCPECGKSFSSKKALTEHQRTHTGEKPYKCPECGKSFSTSHSLTEHQRTHTHPRAPIPKPFQYKCPECGKSFSTSGELVRHQRTHTGKKTS

SEQ ID NO: 1056 - protein, 215 aa, synthetic construct
  REGION 1..215: note = source = /note="Description of Artificial Sequence: Syntheticpolypeptide"
LEPGEKPYKCPECGKSFSRSDHLTTHQRTHTGEKPYKCPECGKSFSRSDDLVRHQRTHTGEKPYKCPECGKSFSSPADLTRHQRTHTGEKPYKCPECGKSFSSRRTCRAHQRTHTGEKPYKCPECGKSFSSKKALTEHQRTHTGEKPYKCPECGKSFSTSHSLTEHQRTHTPNPHRRTDPSHKPFQYKCPECGKSFSDPGHLVRHQRTHTGKKTS

SEQ ID NO: 1057 - protein, 233 aa, synthetic construct
  REGION 1..233: note = source = /note="Description of Artificial Sequence: Syntheticpolypeptide"
LEPGEKPYKCPECGKSFSTSGSLVRHQRTHTPNPHRRTDPSHKPFQYKCPECGKSFSRKDNLKNHQRTHTGEKPYKCPECGKSFSQSGDLRRHQRTHTGEKPYKCPECGKSFSQRANLRAHQRTHTGEKPYKCPECGKSFSQKSSLIAHQRTHTHPRAPIPKPFQYKCPECGKSFSRSDKLTEHQRTHTPNPHRRTDPSHKPFQYKCPECGKSFSTTGALTEHQRTHTGKKTS

SEQ ID NO: 1058 - protein, 233 aa, synthetic construct
  REGION 1..233: note = source = /note="Description of Artificial Sequence: Syntheticpolypeptide"
LEPGEKPYKCPECGKSFSQAGHLASHQRTHTPNPHRRTDPSHKPFQYKCPECGKSFSTSGSLVRHQRTHTPNPHRRTDPSHKPFQYKCPECGKSFSRKDNLKNHQRTHTGEKPYKCPECGKSFSQSGDLRRHQRTHTGEKPYKCPECGKSFSQRANLRAHQRTHTGEKPYKCPECGKSFSQKSSLIAHQRTHTHPRAPIPKPFQYKCPECGKSFSRSDKLTEHQRTHTGKKTS

SEQ ID NO: 1059 - protein, 226 aa, synthetic construct
  REGION 1..226: note = source = /note="Description of Artificial Sequence: Syntheticpolypeptide"
LEPGEKPYKCPECGKSFSQAGHLASHQRTHTPNPHRRTDPSHKPFQYKCPECGKSFSTSGSLVRHQRTHTPNPHRRTDPSHKPFQYKCPECGKSFSRKDNLKNHQRTHTGEKPYKCPECGKSFSQSGDLRRHQRTHTGEKPYKCPECGKSFSQRANLRAHQRTHTGEKPYKCPECGKSFSQKSSLIAHQRTHTGEKPYKCPECGKSFSTSGHLVRHQRTHTGKKTS

SEQ ID NO: 1060 - protein, 233 aa, synthetic construct
  REGION 1..233: note = source = /note="Description of Artificial Sequence: Syntheticpolypeptide"
LEPGEKPYKCPECGKSFSRTDTLRDHQRTHTHPRAPIPKPFQYKCPECGKSFSQAGHLASHQRTHTPNPHRRTDPSHKPFQYKCPECGKSFSTSGSLVRHQRTHTPNPHRRTDPSHKPFQYKCPECGKSFSRKDNLKNHQRTHTGEKPYKCPECGKSFSQSGDLRRHQRTHTGEKPYKCPECGKSFSQRANLRAHQRTHTGEKPYKCPECGKSFSQKSSLIAHQRTHTGKKTS

SEQ ID NO: 1061 - protein, 233 aa, synthetic construct
  REGION 1..233: note = source = /note="Description of Artificial Sequence: Syntheticpolypeptide"
LEPGEKPYKCPECGKSFSQAGHLASHQRTHTGEKPYKCPECGKSFSRTDTLRDHQRTHTHPRAPIPKPFQYKCPECGKSFSQAGHLASHQRTHTPNPHRRTDPSHKPFQYKCPECGKSFSTSGSLVRHQRTHTPNPHRRTDPSHKPFQYKCPECGKSFSRKDNLKNHQRTHTGEKPYKCPECGKSFSQSGDLRRHQRTHTGEKPYKCPECGKSFSQRANLRAHQRTHTGKKTS

SEQ ID NO: 1062 - protein, 233 aa, synthetic construct
  REGION 1..233: note = source = /note="Description of Artificial Sequence: Syntheticpolypeptide"
LEPGEKPYKCPECGKSFSRADNLTEHQRTHTGEKPYKCPECGKSFSQAGHLASHQRTHTGEKPYKCPECGKSFSRTDTLRDHQRTHTHPRAPIPKPFQYKCPECGKSFSQAGHLASHQRTHTPNPHRRTDPSHKPFQYKCPECGKSFSTSGSLVRHQRTHTPNPHRRTDPSHKPFQYKCPECGKSFSRKDNLKNHQRTHTGEKPYKCPECGKSFSQSGDLRRHQRTHTGKKTS

SEQ ID NO: 1063 - protein, 222 aa, synthetic construct
  REGION 1..222: note = source = /note="Description of Artificial Sequence: Syntheticpolypeptide"
LEPGEKPYKCPECGKSFSQAGHLASHQRTHTPNPHRRTDPSHKPFQYKCPECGKSFSTSGSLVRHQRTHTGEKPYKCPECGKSFSTSGSLVRHQRTHTGEKPYKCPECGKSFSQRANLRAHQRTHTGEKPYKCPECGKSFSERSHLREHQRTHTGEKPYKCPECGKSFSQRANLRAHQRTHTHPRAPIPKPFQYKCPECGKSFSQSSSLVRHQRTHTGKKTS

SEQ ID NO: 1064 - protein, 222 aa, synthetic construct
  REGION 1..222: note = source = /note="Description of Artificial Sequence: Syntheticpolypeptide"
LEPGEKPYKCPECGKSFSRTDTLRDHQRTHTHPRAPIPKPFQYKCPECGKSFSQAGHLASHQRTHTPNPHRRTDPSHKPFQYKCPECGKSFSTSGSLVRHQRTHTGEKPYKCPECGKSFSTSGSLVRHQRTHTGEKPYKCPECGKSFSQRANLRAHQRTHTGEKPYKCPECGKSFSERSHLREHQRTHTGEKPYKCPECGKSFSQRANLRAHQRTHTGKKTS

SEQ ID NO: 1065 - protein, 222 aa, synthetic construct
  REGION 1..222: note = source = /note="Description of Artificial Sequence: Syntheticpolypeptide"
LEPGEKPYKCPECGKSFSQAGHLASHQRTHTGEKPYKCPECGKSFSRTDTLRDHQRTHTHPRAPIPKPFQYKCPECGKSFSQAGHLASHQRTHTPNPHRRTDPSHKPFQYKCPECGKSFSTSGSLVRHQRTHTGEKPYKCPECGKSFSTSGSLVRHQRTHTGEKPYKCPECGKSFSQRANLRAHQRTHTGEKPYKCPECGKSFSERSHLREHQRTHTGKKTS

SEQ ID NO: 1066 - protein, 222 aa, synthetic construct
  REGION 1..222: note = source = /note="Description of Artificial Sequence: Syntheticpolypeptide"
LEPGEKPYKCPECGKSFSRADNLTEHQRTHTGEKPYKCPECGKSFSQAGHLASHQRTHTGEKPYKCPECGKSFSRTDTLRDHQRTHTHPRAPIPKPFQYKCPECGKSFSQAGHLASHQRTHTPNPHRRTDPSHKPFQYKCPECGKSFSTSGSLVRHQRTHTGEKPYKCPECGKSFSTSGSLVRHQRTHTGEKPYKCPECGKSFSQRANLRAHQRTHTGKKTS

SEQ ID NO: 1067 - protein, 229 aa, synthetic construct
  REGION 1..229: note = source = /note="Description of Artificial Sequence: Syntheticpolypeptide"
LEPGEKPYKCPECGKSFSQAGHLASHQRTHTGEKPYKCPECGKSFSRTDTLRDHQRTHTHPRAPIPKPFQYKCPECGKSFSQAGHLASHQRTHTPNPHRRTDPSHKPFQYKCPECGKSFSTSGSLVRHQRTHTHPRAPIPKPFQYKCPECGKSFSHRTTLTNHQRTHTGEKPYKCPECGKSFSQSGNLTEHQRTHTGEKPYKCPECGKSFSRKDNLKNHQRTHTGKKTS

SEQ ID NO: 1068 - protein, 229 aa, synthetic construct
  REGION 1..229: note = source = /note="Description of Artificial Sequence: Syntheticpolypeptide"
LEPGEKPYKCPECGKSFSRADNLTEHQRTHTGEKPYKCPECGKSFSQAGHLASHQRTHTGEKPYKCPECGKSFSRTDTLRDHQRTHTHPRAPIPKPFQYKCPECGKSFSQAGHLASHQRTHTPNPHRRTDPSHKPFQYKCPECGKSFSTSGSLVRHQRTHTHPRAPIPKPFQYKCPECGKSFSHRTTLTNHQRTHTGEKPYKCPECGKSFSQSGNLTEHQRTHTGKKTS

SEQ ID NO: 1069 - protein, 215 aa, synthetic construct
  REGION 1..215: note = source = /note="Description of Artificial Sequence: Syntheticpolypeptide"
LEPGEKPYKCPECGKSFSERSHLREHQRTHTGEKPYKCPECGKSFSSPADLTRHQRTHTGEKPYKCPECGKSFSSKKHLAEHQRTHTPNPHRRTDPSHKPFQYKCPECGKSFSHRTTLTNHQRTHTGEKPYKCPECGKSFSDPGNLVRHQRTHTGEKPYKCPECGKSFSSRRTCRAHQRTHTGEKPYKCPECGKSFSQKSSLIAHQRTHTGKKTS

SEQ ID NO: 1070 - protein, 215 aa, synthetic construct
  REGION 1..215: note = source = /note="Description of Artificial Sequence: Syntheticpolypeptide"
LEPGEKPYKCPECGKSFSRADNLTEHQRTHTGEKPYKCPECGKSFSERSHLREHQRTHTGEKPYKCPECGKSFSSPADLTRHQRTHTGEKPYKCPECGKSFSSKKHLAEHQRTHTPNPHRRTDPSHKPFQYKCPECGKSFSHRTTLTNHQRTHTGEKPYKCPECGKSFSDPGNLVRHQRTHTGEKPYKCPECGKSFSSRRTCRAHQRTHTGKKTS

SEQ ID NO: 1071 - protein, 215 aa, synthetic construct
  REGION 1..215: note = source = /note="Description of Artificial Sequence: Syntheticpolypeptide"
LEPGEKPYKCPECGKSFSTSGELVRHQRTHTGEKPYKCPECGKSFSRADNLTEHQRTHTGEKPYKCPECGKSFSERSHLREHQRTHTGEKPYKCPECGKSFSSPADLTRHQRTHTGEKPYKCPECGKSFSSKKHLAEHQRTHTPNPHRRTDPSHKPFQYKCPECGKSFSHRTTLTNHQRTHTGEKPYKCPECGKSFSDPGNLVRHQRTHTGKKTS

SEQ ID NO: 1072 - protein, 215 aa, synthetic construct
  REGION 1..215: note = source = /note="Description of Artificial Sequence: Syntheticpolypeptide"
LEPGEKPYKCPECGKSFSTSGNLVRHQRTHTGEKPYKCPECGKSFSTSGELVRHQRTHTGEKPYKCPECGKSFSRADNLTEHQRTHTGEKPYKCPECGKSFSERSHLREHQRTHTGEKPYKCPECGKSFSSPADLTRHQRTHTGEKPYKCPECGKSFSSKKHLAEHQRTHTPNPHRRTDPSHKPFQYKCPECGKSFSHRTTLTNHQRTHTGKKTS

SEQ ID NO: 1073 - protein, 211 aa, synthetic construct
  REGION 1..211: note = source = /note="Description of Artificial Sequence: Syntheticpolypeptide"
LEPGEKPYKCPECGKSFSRADNLTEHQRTHTGEKPYKCPECGKSFSERSHLREHQRTHTGEKPYKCPECGKSFSSPADLTRHQRTHTGEKPYKCPECGKSFSSKKHLAEHQRTHTHPRAPIPKPFQYKCPECGKSFSTSGSLVRHQRTHTGEKPYKCPECGKSFSSPADLTRHQRTHTGEKPYKCPECGKSFSRSDELVRHQRTHTGKKTS

SEQ ID NO: 1074 - protein, 211 aa, synthetic construct
  REGION 1..211: note = source = /note="Description of Artificial Sequence: Syntheticpolypeptide"
LEPGEKPYKCPECGKSFSTSGELVRHQRTHTGEKPYKCPECGKSFSRADNLTEHQRTHTGEKPYKCPECGKSFSERSHLREHQRTHTGEKPYKCPECGKSFSSPADLTRHQRTHTGEKPYKCPECGKSFSSKKHLAEHQRTHTHPRAPIPKPFQYKCPECGKSFSTSGSLVRHQRTHTGEKPYKCPECGKSFSSPADLTRHQRTHTGKKTS

SEQ ID NO: 1075 - protein, 211 aa, synthetic construct
  REGION 1..211: note = source = /note="Description of Artificial Sequence: Syntheticpolypeptide"
LEPGEKPYKCPECGKSFSTSGNLVRHQRTHTGEKPYKCPECGKSFSTSGELVRHQRTHTGEKPYKCPECGKSFSRADNLTEHQRTHTGEKPYKCPECGKSFSERSHLREHQRTHTGEKPYKCPECGKSFSSPADLTRHQRTHTGEKPYKCPECGKSFSSKKHLAEHQRTHTHPRAPIPKPFQYKCPECGKSFSTSGSLVRHQRTHTGKKTS

SEQ ID NO: 1076 - protein, 204 aa, synthetic construct
  REGION 1..204: note = source = /note="Description of Artificial Sequence: Syntheticpolypeptide"
LEPGEKPYKCPECGKSFSTKNSLTEHQRTHTGEKPYKCPECGKSFSTSGNLVRHQRTHTGEKPYKCPECGKSFSTSGELVRHQRTHTGEKPYKCPECGKSFSRADNLTEHQRTHTGEKPYKCPECGKSFSERSHLREHQRTHTGEKPYKCPECGKSFSSPADLTRHQRTHTGEKPYKCPECGKSFSSKKHLAEHQRTHTGKKTS

SEQ ID NO: 1077 - protein, 229 aa, synthetic construct
  REGION 1..229: note = source = /note="Description of Artificial Sequence: Syntheticpolypeptide"
LEPGEKPYKCPECGKSFSQSGNLTEHQRTHTGEKPYKCPECGKSFSSKKALTEHQRTHTGEKPYKCPECGKSFSTTGALTEHQRTHTGEKPYKCPECGKSFSTSHSLTEHQRTHTHPRAPIPKPFQYKCPECGKSFSRADNLTEHQRTHTHPRAPIPKPFQYKCPECGKSFSRNDALTEHQRTHTPNPHRRTDPSHKPFQYKCPECGKSFSTTGALTEHQRTHTGKKTS

SEQ ID NO: 1078 - protein, 218 aa, synthetic construct
  REGION 1..218: note = source = /note="Description of Artificial Sequence: Syntheticpolypeptide"
LEPGEKPYKCPECGKSFSTSGHLVRHQRTHTGEKPYKCPECGKSFSQSGNLTEHQRTHTGEKPYKCPECGKSFSSKKALTEHQRTHTGEKPYKCPECGKSFSTTGALTEHQRTHTGEKPYKCPECGKSFSTSHSLTEHQRTHTHPRAPIPKPFQYKCPECGKSFSRADNLTEHQRTHTHPRAPIPKPFQYKCPECGKSFSRNDALTEHQRTHTGKKTS

SEQ ID NO: 1079 - protein, 204 aa, synthetic construct
  REGION 1..204: note = source = /note="Description of Artificial Sequence: Syntheticpolypeptide"
LEPGEKPYKCPECGKSFSTSGHLVRHQRTHTGEKPYKCPECGKSFSQSGNLTEHQRTHTGEKPYKCPECGKSFSSKKALTEHQRTHTGEKPYKCPECGKSFSTTGALTEHQRTHTGEKPYKCPECGKSFSTSHSLTEHQRTHTGEKPYKCPECGKSFSHRTTLTNHQRTHTGEKPYKCPECGKSFSDPGALVRHQRTHTGKKTS

SEQ ID NO: 1080 - protein, 204 aa, synthetic construct
  REGION 1..204: note = source = /note="Description of Artificial Sequence: Syntheticpolypeptide"
LEPGEKPYKCPECGKSFSRSDNLVRHQRTHTGEKPYKCPECGKSFSTSGHLVRHQRTHTGEKPYKCPECGKSFSQSGNLTEHQRTHTGEKPYKCPECGKSFSSKKALTEHQRTHTGEKPYKCPECGKSFSTTGALTEHQRTHTGEKPYKCPECGKSFSTSHSLTEHQRTHTGEKPYKCPECGKSFSHRTTLTNHQRTHTGKKTS

SEQ ID NO: 1081 - protein, 211 aa, synthetic construct
  REGION 1..211: note = source = /note="Description of Artificial Sequence: Syntheticpolypeptide"
LEPGEKPYKCPECGKSFSDPGALVRHQRTHTHPRAPIPKPFQYKCPECGKSFSRSDNLVRHQRTHTGEKPYKCPECGKSFSTSGHLVRHQRTHTGEKPYKCPECGKSFSQSGNLTEHQRTHTGEKPYKCPECGKSFSSKKALTEHQRTHTGEKPYKCPECGKSFSTTGALTEHQRTHTGEKPYKCPECGKSFSTSHSLTEHQRTHTGKKTS

SEQ ID NO: 1082 - protein, 211 aa, synthetic construct
  REGION 1..211: note = source = /note="Description of Artificial Sequence: Syntheticpolypeptide"
LEPGEKPYKCPECGKSFSTKNSLTEHQRTHTGEKPYKCPECGKSFSDPGALVRHQRTHTHPRAPIPKPFQYKCPECGKSFSRSDNLVRHQRTHTGEKPYKCPECGKSFSTSGHLVRHQRTHTGEKPYKCPECGKSFSQSGNLTEHQRTHTGEKPYKCPECGKSFSSKKALTEHQRTHTGEKPYKCPECGKSFSTTGALTEHQRTHTGKKTS

SEQ ID NO: 1083 - protein, 211 aa, synthetic construct
  REGION 1..211: note = source = /note="Description of Artificial Sequence: Syntheticpolypeptide"
LEPGEKPYKCPECGKSFSTSGELVRHQRTHTGEKPYKCPECGKSFSTKNSLTEHQRTHTGEKPYKCPECGKSFSDPGALVRHQRTHTHPRAPIPKPFQYKCPECGKSFSRSDNLVRHQRTHTGEKPYKCPECGKSFSTSGHLVRHQRTHTGEKPYKCPECGKSFSQSGNLTEHQRTHTGEKPYKCPECGKSFSSKKALTEHQRTHTGKKTS

SEQ ID NO: 1084 - protein, 222 aa, synthetic construct
  REGION 1..222: note = source = /note="Description of Artificial Sequence: Syntheticpolypeptide"
LEPGEKPYKCPECGKSFSSKKHLAEHQRTHTPNPHRRTDPSHKPFQYKCPECGKSFSTSGELVRHQRTHTGEKPYKCPECGKSFSTKNSLTEHQRTHTGEKPYKCPECGKSFSDPGALVRHQRTHTHPRAPIPKPFQYKCPECGKSFSRSDNLVRHQRTHTGEKPYKCPECGKSFSTSGHLVRHQRTHTGEKPYKCPECGKSFSQSGNLTEHQRTHTGKKTS

SEQ ID NO: 1085 - protein, 222 aa, synthetic construct
  REGION 1..222: note = source = /note="Description of Artificial Sequence: Syntheticpolypeptide"
LEPGEKPYKCPECGKSFSQNSTLTEHQRTHTGEKPYKCPECGKSFSSKKHLAEHQRTHTPNPHRRTDPSHKPFQYKCPECGKSFSTSGELVRHQRTHTGEKPYKCPECGKSFSTKNSLTEHQRTHTGEKPYKCPECGKSFSDPGALVRHQRTHTHPRAPIPKPFQYKCPECGKSFSRSDNLVRHQRTHTGEKPYKCPECGKSFSTSGHLVRHQRTHTGKKTS

SEQ ID NO: 1086 - protein, 222 aa, synthetic construct
  REGION 1..222: note = source = /note="Description of Artificial Sequence: Syntheticpolypeptide"
LEPGEKPYKCPECGKSFSTSHSLTEHQRTHTGEKPYKCPECGKSFSQNSTLTEHQRTHTGEKPYKCPECGKSFSSKKHLAEHQRTHTPNPHRRTDPSHKPFQYKCPECGKSFSTSGELVRHQRTHTGEKPYKCPECGKSFSTKNSLTEHQRTHTGEKPYKCPECGKSFSDPGALVRHQRTHTHPRAPIPKPFQYKCPECGKSFSRSDNLVRHQRTHTGKKTS

SEQ ID NO: 1087 - protein, 211 aa, synthetic construct
  REGION 1..211: note = source = /note="Description of Artificial Sequence: Syntheticpolypeptide"
LEPGEKPYKCPECGKSFSRSDNLVRHQRTHTGEKPYKCPECGKSFSTSGHLVRHQRTHTGEKPYKCPECGKSFSQSGNLTEHQRTHTGEKPYKCPECGKSFSSKKALTEHQRTHTGEKPYKCPECGKSFSTTGALTEHQRTHTGEKPYKCPECGKSFSTSHSLTEHQRTHTHPRAPIPKPFQYKCPECGKSFSRADNLTEHQRTHTGKKTS

SEQ ID NO: 1088 - protein, 222 aa, synthetic construct
  REGION 1..222: note = source = /note="Description of Artificial Sequence: Syntheticpolypeptide"
LEPGEKPYKCPECGKSFSDPGALVRHQRTHTHPRAPIPKPFQYKCPECGKSFSRSDNLVRHQRTHTGEKPYKCPECGKSFSTSGHLVRHQRTHTGEKPYKCPECGKSFSQSGNLTEHQRTHTGEKPYKCPECGKSFSSKKALTEHQRTHTPNPHRRTDPSHKPFQYKCPECGKSFSSKKALTEHQRTHTGEKPYKCPECGKSFSDPGALVRHQRTHTGKKTS

SEQ ID NO: 1089 - protein, 222 aa, synthetic construct
  REGION 1..222: note = source = /note="Description of Artificial Sequence: Syntheticpolypeptide"
LEPGEKPYKCPECGKSFSTKNSLTEHQRTHTGEKPYKCPECGKSFSDPGALVRHQRTHTHPRAPIPKPFQYKCPECGKSFSRSDNLVRHQRTHTGEKPYKCPECGKSFSTSGHLVRHQRTHTGEKPYKCPECGKSFSQSGNLTEHQRTHTGEKPYKCPECGKSFSSKKALTEHQRTHTPNPHRRTDPSHKPFQYKCPECGKSFSSKKALTEHQRTHTGKKTS

SEQ ID NO: 1090 - protein, 218 aa, synthetic construct
  REGION 1..218: note = source = /note="Description of Artificial Sequence: Syntheticpolypeptide"
LEPGEKPYKCPECGKSFSTKNSLTEHQRTHTGEKPYKCPECGKSFSDPGALVRHQRTHTHPRAPIPKPFQYKCPECGKSFSRSDNLVRHQRTHTGEKPYKCPECGKSFSTSGHLVRHQRTHTGEKPYKCPECGKSFSQSGNLTEHQRTHTGEKPYKCPECGKSFSSKKALTEHQRTHTHPRAPIPKPFQYKCPECGKSFSTHLDLIRHQRTHTGKKTS